SEQUENCE LISTING (885 sequences)

SEQ ID NO: 1 - protein, 124 aa, synthetic construct
  REGION 1..124: note = antibody sequence
EVQLLESGGGLVQPGGSLRLSCAASGFTFSSYPMGWVRQAPGKGLEWVAGIDDDGDSDTRYAPAVKGRATISRDNSKNTVYLQMNSLRAEDTAVYYCAKHTGIGANSAGSIDAWGQGTLVTVSS

SEQ ID NO: 2 - protein, 5 aa, synthetic construct
  REGION 1..5: note = antibody sequence
SYPMG

SEQ ID NO: 3 - protein, 18 aa, synthetic construct
  REGION 1..18: note = antibody sequence
GIDDDGDSDTRYAPAVKG

SEQ ID NO: 4 - protein, 14 aa, synthetic construct
  REGION 1..14: note = antibody sequence
HTGIGANSAGSIDA

SEQ ID NO: 5 - protein, 108 aa, synthetic construct
  REGION 1..108: note = antibody sequence
SYELTQPPSVSVSPGQTARITCSGGGSYTGSYYYGWYQQKPGQAPVTVIYYNNKRPSDIPERFSGSLSGTTNTLTISGVQAEDEADYYCGSADNSGDAFGTGTKVTVL

SEQ ID NO: 6 - protein, 13 aa, synthetic construct
  REGION 1..13: note = antibody sequence
SGGGSYTGSYYYG

SEQ ID NO: 7 - protein, 7 aa, synthetic construct
  REGION 1..7: note = antibody sequence
YNNKRPS

SEQ ID NO: 8 - protein, 9 aa, synthetic construct
  REGION 1..9: note = antibody sequence
GSADNSGDA

SEQ ID NO: 9 - DNA, 372 bp, synthetic construct
  misc_feature 1..372: note = antibody sequence
  source 1..372: mol_type = other DNA
gaagttcagctgctggaatctggcggcggactggttcaacctggcggatctctgagactgagctgtgccgccagcggcttcacctttagcagctatcctatgggctgggtccgacaggcccctggcaaaggacttgaatgggtggccggcatcgacgacgatggcgatagcgatacaagatacgcccctgccgtgaagggcagagccaccatctccagagacaacagcaagaacaccgtgtacctgcagatgaacagcctgagagccgaggacaccgccgtgtactattgtgccaagcacacaggcatcggcgccaattctgccggctctattgatgcctggggccagggaacactggtcacagtttcttca

SEQ ID NO: 10 - DNA, 15 bp, synthetic construct
  misc_feature 1..15: note = antibody sequence
  source 1..15: mol_type = other DNA
agctatcctatgggc

SEQ ID NO: 11 - DNA, 54 bp, synthetic construct
  misc_feature 1..54: note = antibody sequence
  source 1..54: mol_type = other DNA
ggcatcgacgacgatggcgatagcgatacaagatacgcccctgccgtgaagggc

SEQ ID NO: 12 - DNA, 42 bp, synthetic construct
  misc_feature 1..42: note = antibody sequence
  source 1..42: mol_type = other DNA
cacacaggcatcggcgccaattctgccggctctattgatgcc

SEQ ID NO: 13 - DNA, 324 bp, synthetic construct
  misc_feature 1..324: note = antibody sequence
  source 1..324: mol_type = other DNA
agctatgagctgacacagcctccaagcgtgtccgtgtctcctggacagaccgccagaatcacatgtagcggcggaggcagctacaccggcagctactactatggctggtatcagcagaagcccggacaggcccctgtgaccgtgatctactacaacaacaagcggcccagcgacatccccgagagattttctggctctctgagcggcaccaccaacacactgacaatctctggcgtgcaggccgaggacgaggccgattactattgtggcagcgccgataatagcggcgacgcctttggcaccggcaccaaagttacagtgcta

SEQ ID NO: 14 - DNA, 39 bp, synthetic construct
  misc_feature 1..39: note = antibody sequence
  source 1..39: mol_type = other DNA
agcggcggaggcagctacaccggcagctactactatggc

SEQ ID NO: 15 - DNA, 21 bp, synthetic construct
  misc_feature 1..21: note = antibody sequence
  source 1..21: mol_type = other DNA
tacaacaacaagcggcccagc

SEQ ID NO: 16 - DNA, 27 bp, synthetic construct
  misc_feature 1..27: note = antibody sequence
  source 1..27: mol_type = other DNA
ggcagcgccgataatagcggcgacgcc

SEQ ID NO: 17 - protein, 453 aa, synthetic construct
  REGION 1..453: note = antibody sequence
EVQLLESGGGLVQPGGSLRLSCAASGFTFSSYPMGWVRQAPGKGLEWVAGIDDDGDSDTRYAPAVKGRATISRDNSKNTVYLQMNSLRAEDTAVYYCAKHTGIGANSAGSIDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 18 - protein, 214 aa, synthetic construct
  REGION 1..214: note = antibody sequence
SYELTQPPSVSVSPGQTARITCSGGGSYTGSYYYGWYQQKPGQAPVTVIYYNNKRPSDIPERFSGSLSGTTNTLTISGVQAEDEADYYCGSADNSGDAFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 19 - DNA, 1359 bp, synthetic construct
  misc_feature 1..1359: note = antibody sequence
  source 1..1359: mol_type = other DNA
gaagttcagctgctggaatctggcggcggactggttcaacctggcggatctctgagactgagctgtgccgccagcggcttcacctttagcagctatcctatgggctgggtccgacaggcccctggcaaaggacttgaatgggtggccggcatcgacgacgatggcgatagcgatacaagatacgcccctgccgtgaagggcagagccaccatctccagagacaacagcaagaacaccgtgtacctgcagatgaacagcctgagagccgaggacaccgccgtgtactattgtgccaagcacacaggcatcggcgccaattctgccggctctattgatgcctggggccagggaacactggtcacagtttcttcagccagcaccaagggccccagcgtgttccctctggcccctagcagcaagagcacatctggcggaacagccgccctgggctgcctcgtgaaggactactttcccgagcccgtgaccgtgtcctggaactctggcgctctgacaagcggcgtgcacacctttccagccgtgctgcagagcagcggcctgtactctctgagcagcgtcgtgacagtgcccagcagctctctgggcacccagacctacatctgcaacgtgaaccacaagcccagcaacaccaaggtggacaagaaggtggaacccaagagctgcgacaagacccacacctgtcccccttgtcctgcccccgaactgctgggaggcccttccgtgttcctgttccccccaaagcccaaggacaccctgatgatcagccggacccccgaagtgacctgcgtggtggtggatgtgtcccacgaggaccctgaagtgaagttcaattggtacgtggacggcgtggaagtgcacaacgccaagaccaagcctagagaggaacagtacaacagcacctaccgggtggtgtccgtgctgacagtgctgcaccaggactggctgaacggcaaagagtacaagtgcaaggtgtccaacaaggccctgcctgcccccatcgagaaaaccatcagcaaggccaagggccagccccgcgaaccccaggtgtacacactgcccccaagcagggacgagctgaccaagaaccaggtgtccctgacctgtctcgtgaaaggcttctacccctccgatatcgccgtggaatgggagagcaacggccagcccgagaacaactacaagaccaccccccctgtgctggacagcgacggctcattcttcctgtacagcaagctgaccgtggacaagtcccggtggcagcagggcaacgtgttcagctgcagcgtgatgcacgaggccctgcacaaccactacacccagaagtccctgagcctgagccctggc

SEQ ID NO: 20 - DNA, 642 bp, synthetic construct
  misc_feature 1..642: note = antibody sequence
  source 1..642: mol_type = other DNA
agctatgagctgacacagcctccaagcgtgtccgtgtctcctggacagaccgccagaatcacatgtagcggcggaggcagctacaccggcagctactactatggctggtatcagcagaagcccggacaggcccctgtgaccgtgatctactacaacaacaagcggcccagcgacatccccgagagattttctggctctctgagcggcaccaccaacacactgacaatctctggcgtgcaggccgaggacgaggccgattactattgtggcagcgccgataatagcggcgacgcctttggcaccggcaccaaagttacagtgctaggccagcctaaagccgcccctagcgtgaccctgttccctccaagcagcgaggaactgcaggccaacaaggccaccctcgtgtgcctgatcagcgacttctatcctggcgccgtgaccgtggcctggaaggccgatagctctcctgtgaaggccggcgtggaaaccaccacccctagcaagcagagcaacaacaaatacgccgccagcagctacctgagcctgacccccgagcagtggaagtcccacagatcctacagctgccaagtgacccacgagggcagcaccgtggaaaagacagtggcccctaccgagtgcagc

SEQ ID NO: 21 - protein, 124 aa, synthetic construct; identical to SEQ ID NO: 1
  REGION 1..124: note = antibody sequence

SEQ ID NO: 22 - protein, 5 aa, synthetic construct
  REGION 1..5: note = antibody sequence
SYPMG

SEQ ID NO: 23 - protein, 18 aa, synthetic construct
  REGION 1..18: note = antibody sequence
GIDDDGDSDTRYAPAVKG

SEQ ID NO: 24 - protein, 14 aa, synthetic construct
  REGION 1..14: note = antibody sequence
HTGIGANSAGSIDA

SEQ ID NO: 25 - protein, 108 aa, synthetic construct; identical to SEQ ID NO: 5
  REGION 1..108: note = antibody sequence

SEQ ID NO: 26 - protein, 13 aa, synthetic construct
  REGION 1..13: note = antibody sequence
SGGGSYTGSYYYG

SEQ ID NO: 27 - protein, 7 aa, synthetic construct
  REGION 1..7: note = antibody sequence
YNNKRPS

SEQ ID NO: 28 - protein, 9 aa, synthetic construct
  REGION 1..9: note = antibody sequence
GSADNSGDA

SEQ ID NO: 29 - DNA, 372 bp, synthetic construct; identical to SEQ ID NO: 9
  misc_feature 1..372: note = antibody sequence
  source 1..372: mol_type = other DNA

SEQ ID NO: 30 - DNA, 15 bp, synthetic construct
  misc_feature 1..15: note = antibody sequence
  source 1..15: mol_type = other DNA
agctatcctatgggc

SEQ ID NO: 31 - DNA, 54 bp, synthetic construct; identical to SEQ ID NO: 11
  misc_feature 1..54: note = antibody sequence
  source 1..54: mol_type = other DNA

SEQ ID NO: 32 - DNA, 42 bp, synthetic construct; identical to SEQ ID NO: 12
  misc_feature 1..42: note = antibody sequence
  source 1..42: mol_type = other DNA

SEQ ID NO: 33 - DNA, 324 bp, synthetic construct; identical to SEQ ID NO: 13
  misc_feature 1..324: note = antibody sequence
  source 1..324: mol_type = other DNA

SEQ ID NO: 34 - DNA, 39 bp, synthetic construct; identical to SEQ ID NO: 14
  misc_feature 1..39: note = antibody sequence
  source 1..39: mol_type = other DNA

SEQ ID NO: 35 - DNA, 21 bp, synthetic construct
  misc_feature 1..21: note = antibody sequence
  source 1..21: mol_type = other DNA
tacaacaacaagcggcccagc

SEQ ID NO: 36 - DNA, 27 bp, synthetic construct
  misc_feature 1..27: note = antibody sequence
  source 1..27: mol_type = other DNA
ggcagcgccgataatagcggcgacgcc

SEQ ID NO: 37 - protein, 448 aa, synthetic construct
  REGION 1..448: note = antibody sequence
EVQLLESGGGLVQPGGSLRLSCAASGFTFSSYPMGWVRQAPGKGLEWVAGIDDDGDSDTRYAPAVKGRATISRDNSKNTVYLQMNSLRAEDTAVYYCAKHTGIGANSAGSIDAWGQGTLVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 38 - protein, 214 aa, synthetic construct
  REGION 1..214: note = antibody sequence
SYELTQPPSVSVSPGQTARITCSGGGSYTGSYYYGWYQQKPGQAPVTVIYYNNKRPSDIPERFSGSLSGTTNTLTISGVQAEDEADYYCGSADNSGDAFGTGTKVTVLGQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

SEQ ID NO: 39 - DNA, 1344 bp, synthetic construct
  misc_feature 1..1344: note = antibody sequence
  source 1..1344: mol_type = other DNA
gaagttcagctgctggaatctggcggcggactggttcaacctggcggatctctgagactgagctgtgccgccagcggcttcacctttagcagctatcctatgggctgggtccgacaggcccctggcaaaggacttgaatgggtggccggcatcgacgacgatggcgatagcgatacaagatacgcccctgccgtgaagggcagagccaccatctccagagacaacagcaagaacaccgtgtacctgcagatgaacagcctgagagccgaggacaccgccgtgtactattgtgccaagcacacaggcatcggcgccaattctgccggctctattgatgcctggggccagggaacactggtcacagtttcttcagccaagaccaccccccccagcgtgtaccctctggctcctggatctgccgcccagaccaacagcatggtcaccctgggctgcctcgtgaagggctacttccctgagcctgtgaccgtgacctggaacagcggctctctgtctagcggcgtgcacacctttccagccgtgctgcagagcgacctgtacaccctgagcagcagcgtgaccgtgcctagcagcacctggcctagcgagacagtgacctgcaacgtggcccaccctgccagcagcacaaaggtggacaagaaaatcgtgccccgggactgcggctgcaagccctgtatctgtaccgtgcccgaggtgtccagcgtgttcatcttcccacccaagcccaaggacgtgctgaccatcaccctgacccccaaagtgacctgtgtggtggtggacatcagcaaggacgaccccgaggtgcagttcagttggttcgtggacgacgtggaagtgcacacagcccagacccagcccagagaggaacagttcaacagcaccttcagaagcgtgtccgagctgcccatcatgcaccaggactggctgaacggcaaagagttcaagtgcagagtgaacagcgccgccttccctgcccccatcgagaaaaccatctccaagaccaagggcagacccaaggcccctcaggtgtacacaatccccccacccaaagaacagatggccaaggacaaggtgtccctgacctgcatgatcaccgatttcttcccagaggacatcaccgtggaatggcagtggaacggccagcccgccgagaactacaagaacacccagcctatcatggacaccgacggcagctacttcgtgtacagcaagctgaacgtgcagaagtccaactgggaggccggcaacaccttcacctgtagcgtgctgcacgagggcctgcacaatcaccacaccgagaagtccctgtcccacagccctggcaag

SEQ ID NO: 40 - DNA, 642 bp, synthetic construct
  misc_feature 1..642: note = antibody sequence
  source 1..642: mol_type = other DNA
agctatgagctgacacagcctccaagcgtgtccgtgtctcctggacagaccgccagaatcacatgtagcggcggaggcagctacaccggcagctactactatggctggtatcagcagaagcccggacaggcccctgtgaccgtgatctactacaacaacaagcggcccagcgacatccccgagagattttctggctctctgagcggcaccaccaacacactgacaatctctggcgtgcaggccgaggacgaggccgattactattgtggcagcgccgataatagcggcgacgcctttggcaccggcaccaaagttacagtgctaggccagcccaagagcagccctagcgtgaccctgttccctccaagcagcgaggaactggaaacaaacaaggccaccctcgtgtgcaccatcaccgacttctaccccggcgtcgtgaccgtggactggaaggtggacggcaccccagtgacccagggcatggaaaccacccagcccagcaagcagagcaacaacaagtacatggccagcagctacctgaccctgaccgccagagcctgggagagacacagctcctacagctgccaagtgacccacgagggccacaccgtggaaaagagcctgagcagagccgactgcagc

SEQ ID NO: 41 - protein, 120 aa, synthetic construct
  REGION 1..120: note = antibody sequence
EVQLLESGGGLVQPGGSLRLSCAASGFTFSSYGMHWVRQAPGKGLEWVSAIGTGGDTYYADSVMGRFTISRDNSKNTLYLQMNSLRAEDTAVYYCARRDDYTSRDAFDVWGQGTLVTVSS

SEQ ID NO: 42 - protein, 5 aa, synthetic construct
  REGION 1..5: note = antibody sequence
SYGMH

SEQ ID NO: 43 - protein, 16 aa, synthetic construct
  REGION 1..16: note = antibody sequence
AIGTGGDTYYADSVMG

SEQ ID NO: 44 - protein, 12 aa, synthetic construct
  REGION 1..12: note = antibody sequence
RDDYTSRDAFDV

SEQ ID NO: 45 - protein, 111 aa, synthetic construct
  REGION 1..111: note = antibody sequence
QSVLTQPPSASGTPGQRVTISCSGSSSNIGSNTVNWYQQLPGTAPKLLIYYDDLLPSGVPDRFSGSKSGTSASLAISGLRSEDEADYYCAAWDDSLNGYVVFGGGTKLTVL

SEQ ID NO: 46 - protein, 13 aa, synthetic construct
  REGION 1..13: note = antibody sequence
SGSSSNIGSNTVN

SEQ ID NO: 47 - protein, 7 aa, synthetic construct
  REGION 1..7: note = antibody sequence
YDDLLPS

SEQ ID NO: 48 - protein, 12 aa, synthetic construct
  REGION 1..12: note = antibody sequence
AAWDDSLNGYVV

SEQ ID NO: 49 - DNA, 360 bp, synthetic construct
  misc_feature 1..360: note = antibody sequence
  source 1..360: mol_type = other DNA
gaagttcagctgctggaatctggcggcggactggttcaacctggcggatctctgagactgagctgtgccgccagcggcttcacctttagcagctatggcatgcactgggtccgacaggcccctggcaaaggacttgaatgggtgtccgccatcggcacaggcggcgatacctactatgccgatagcgtgatgggcagattcaccatcagccgggacaacagcaagaacaccctgtacctgcagatgaacagcctgagagccgaggacaccgccgtgtactattgcgccagaagggacgactacaccagcagggatgccttcgatgtgtggggccagggaacactggttaccgtttcttca

SEQ ID NO: 50 - DNA, 15 bp, synthetic construct
  misc_feature 1..15: note = antibody sequence
  source 1..15: mol_type = other DNA
agctatggcatgcac

SEQ ID NO: 51 - DNA, 48 bp, synthetic construct
  misc_feature 1..48: note = antibody sequence
  source 1..48: mol_type = other DNA
gccatcggcacaggcggcgatacctactatgccgatagcgtgatgggc

SEQ ID NO: 52 - DNA, 36 bp, synthetic construct
  misc_feature 1..36: note = antibody sequence
  source 1..36: mol_type = other DNA
agggacgactacaccagcagggatgccttcgatgtg

SEQ ID NO: 53 - DNA, 333 bp, synthetic construct
  misc_feature 1..333: note = antibody sequence
  source 1..333: mol_type = other DNA
cagtctgttctgacacagcctcctagcgcctctggcacacctggacagagagtgaccatcagctgtagcggcagcagctccaacatcggcagcaacaccgtgaactggtatcagcagctgcctggcacagcccctaaactgctgatctactacgacgacctgctgcctagcggcgtgcccgatagattttctggcagcaagagcggcacaagcgccagcctggctatctctggactgagatctgaggacgaggccgactactattgcgccgcctgggacgatagcctgaacggctatgtggttttcggcggaggcaccaagctgaccgtgcta

SEQ ID NO: 54 - DNA, 39 bp, synthetic construct
  misc_feature 1..39: note = antibody sequence
  source 1..39: mol_type = other DNA
agcggcagcagctccaacatcggcagcaacaccgtgaac

SEQ ID NO: 55 - DNA, 21 bp, synthetic construct
  misc_feature 1..21: note = antibody sequence
  source 1..21: mol_type = other DNA
tacgacgacctgctgcctagc

SEQ ID NO: 56 - DNA, 36 bp, synthetic construct
  misc_feature 1..36: note = antibody sequence
  source 1..36: mol_type = other DNA
gccgcctgggacgatagcctgaacggctatgtggtt

SEQ ID NO: 57 - protein, 449 aa, synthetic construct
  REGION 1..449: note = antibody sequence
EVQLLESGGGLVQPGGSLRLSCAASGFTFSSYGMHWVRQAPGKGLEWVSAIGTGGDTYYADSVMGRFTISRDNSKNTLYLQMNSLRAEDTAVYYCARRDDYTSRDAFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 58 - protein, 217 aa, synthetic construct
  REGION 1..217: note = antibody sequence
QSVLTQPPSASGTPGQRVTISCSGSSSNIGSNTVNWYQQLPGTAPKLLIYYDDLLPSGVPDRFSGSKSGTSASLAISGLRSEDEADYYCAAWDDSLNGYVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 59 - DNA, 1347 bp, synthetic construct
  misc_feature 1..1347: note = antibody sequence
  source 1..1347: mol_type = other DNA
gaagttcagctgctggaatctggcggcggactggttcaacctggcggatctctgagactgagctgtgccgccagcggcttcacctttagcagctatggcatgcactgggtccgacaggcccctggcaaaggacttgaatgggtgtccgccatcggcacaggcggcgatacctactatgccgatagcgtgatgggcagattcaccatcagccgggacaacagcaagaacaccctgtacctgcagatgaacagcctgagagccgaggacaccgccgtgtactattgcgccagaagggacgactacaccagcagggatgccttcgatgtgtggggccagggaacactggttaccgtttcttcagccagcaccaagggccccagcgtgttccctctggcccctagcagcaagagcacatctggcggaacagccgccctgggctgcctcgtgaaggactactttcccgagcccgtgaccgtgtcctggaactctggcgctctgacaagcggcgtgcacacctttccagccgtgctgcagagcagcggcctgtactctctgagcagcgtcgtgacagtgcccagcagctctctgggcacccagacctacatctgcaacgtgaaccacaagcccagcaacaccaaggtggacaagaaggtggaacccaagagctgcgacaagacccacacctgtcccccttgtcctgcccccgaactgctgggaggcccttccgtgttcctgttccccccaaagcccaaggacaccctgatgatcagccggacccccgaagtgacctgcgtggtggtggatgtgtcccacgaggaccctgaagtgaagttcaattggtacgtggacggcgtggaagtgcacaacgccaagaccaagcctagagaggaacagtacaacagcacctaccgggtggtgtccgtgctgacagtgctgcaccaggactggctgaacggcaaagagtacaagtgcaaggtgtccaacaaggccctgcctgcccccatcgagaaaaccatcagcaaggccaagggccagccccgcgaaccccaggtgtacacactgcccccaagcagggacgagctgaccaagaaccaggtgtccctgacctgtctcgtgaaaggcttctacccctccgatatcgccgtggaatgggagagcaacggccagcccgagaacaactacaagaccaccccccctgtgctggacagcgacggctcattcttcctgtacagcaagctgaccgtggacaagtcccggtggcagcagggcaacgtgttcagctgcagcgtgatgcacgaggccctgcacaaccactacacccagaagtccctgagcctgagccctggc

SEQ ID NO: 60 - DNA, 651 bp, synthetic construct
  misc_feature 1..651: note = antibody sequence
  source 1..651: mol_type = other DNA
cagtctgttctgacacagcctcctagcgcctctggcacacctggacagagagtgaccatcagctgtagcggcagcagctccaacatcggcagcaacaccgtgaactggtatcagcagctgcctggcacagcccctaaactgctgatctactacgacgacctgctgcctagcggcgtgcccgatagattttctggcagcaagagcggcacaagcgccagcctggctatctctggactgagatctgaggacgaggccgactactattgcgccgcctgggacgatagcctgaacggctatgtggttttcggcggaggcaccaagctgaccgtgctaggccagcctaaagccgcccctagcgtgaccctgttccctccaagcagcgaggaactgcaggccaacaaggccaccctcgtgtgcctgatcagcgacttctatcctggcgccgtgaccgtggcctggaaggccgatagctctcctgtgaaggccggcgtggaaaccaccacccctagcaagcagagcaacaacaaatacgccgccagcagctacctgagcctgacccccgagcagtggaagtcccacagatcctacagctgccaagtgacccacgagggcagcaccgtggaaaagacagtggcccctaccgagtgcagc

SEQ ID NO: 61 - protein, 123 aa, synthetic construct
  REGION 1..123: note = antibody sequence
EVQLLESGGGLVQPGGSLRLSCAASGFTFSSYEMNWVRQAPGKGLEWVSGISWNSGSIGYADSVKGRFTISRDNSKNTLYLQMNSLRAEDTAVYYCARSGYSSSWFDPDFDYWGQGTLVTVSS

SEQ ID NO: 62 - protein, 5 aa, synthetic construct
  REGION 1..5: note = antibody sequence
SYEMN

SEQ ID NO: 63 - protein, 17 aa, synthetic construct
  REGION 1..17: note = antibody sequence
GISWNSGSIGYADSVKG

SEQ ID NO: 64 - protein, 14 aa, synthetic construct
  REGION 1..14: note = antibody sequence
SGYSSSWFDPDFDY

SEQ ID NO: 65 - protein, 111 aa, synthetic construct
  REGION 1..111: note = antibody sequence
QSVLTQPPSASGTPGQRVTISCTGSSSNIGAGYDVHWYQQLPGTAPKLLIYGNSNRPSGVPDRFSGSKSGTSASLAISGLRSEDEADYYCSSYAGSNPYVVFGGGTKLTVL

SEQ ID NO: 66 - protein, 14 aa, synthetic construct
  REGION 1..14: note = antibody sequence
TGSSSNIGAGYDVH

SEQ ID NO: 67 - protein, 7 aa, synthetic construct
  REGION 1..7: note = antibody sequence
GNSNRPS

SEQ ID NO: 68 - protein, 11 aa, synthetic construct
  REGION 1..11: note = antibody sequence
SSYAGSNPYVV

SEQ ID NO: 69 - DNA, 369 bp, synthetic construct
  misc_feature 1..369: note = antibody sequence
  source 1..369: mol_type = other DNA
gaagttcagctgctggaatctggcggcggactggttcaacctggcggatctctgagactgagctgtgccgccagcggcttcacctttagcagctacgagatgaactgggtccgacaggcccctggcaaaggccttgaatgggtgtccggcatcagctggaatagcggctctatcggctacgccgacagcgtgaagggcagattcaccatcagccgggacaacagcaagaacaccctgtacctgcagatgaacagcctgagagccgaggacaccgccgtgtactactgtgccagaagcggctacagcagctcttggtttgaccccgacttcgactattggggccagggcacactggtcacagtctcttca

SEQ ID NO: 70 - DNA, 15 bp, synthetic construct
  misc_feature 1..15: note = antibody sequence
  source 1..15: mol_type = other DNA
agctacgagatgaac

SEQ ID NO: 71 - DNA, 51 bp, synthetic construct
  misc_feature 1..51: note = antibody sequence
  source 1..51: mol_type = other DNA
ggcatcagctggaatagcggctctatcggctacgccgacagcgtgaagggc

SEQ ID NO: 72 - DNA, 42 bp, synthetic construct
  misc_feature 1..42: note = antibody sequence
  source 1..42: mol_type = other DNA
agcggctacagcagctcttggtttgaccccgacttcgactat

SEQ ID NO: 73 - DNA, 333 bp, synthetic construct
  misc_feature 1..333: note = antibody sequence
  source 1..333: mol_type = other DNA
cagtctgttctgacacagcctcctagcgcctctggcacacctggacagagagtgaccatcagctgtaccggcagcagctccaatatcggagccggctatgacgtgcactggtatcagcagctgcctggcacagcccctaaactgctgatctacggcaacagcaacagacccagcggcgtgcccgatagattttccggctctaagagcggcacaagcgccagcctggctatctctggactgagatctgaggacgaggccgactactactgcagcagctatgccggcagcaacccctacgttgtgtttggcggaggcaccaagctgaccgttcta

SEQ ID NO: 74 - DNA, 42 bp, synthetic construct
  misc_feature 1..42: note = antibody sequence
  source 1..42: mol_type = other DNA
accggcagcagctccaatatcggagccggctatgacgtgcac

SEQ ID NO: 75 - DNA, 21 bp, synthetic construct
  misc_feature 1..21: note = antibody sequence
  source 1..21: mol_type = other DNA
ggcaacagcaacagacccagc

SEQ ID NO: 76 - DNA, 33 bp, synthetic construct
  misc_feature 1..33: note = antibody sequence
  source 1..33: mol_type = other DNA
agcagctatgccggcagcaacccctacgttgtg

SEQ ID NO: 77 - protein, 452 aa, synthetic construct
  REGION 1..452: note = antibody sequence
EVQLLESGGGLVQPGGSLRLSCAASGFTFSSYEMNWVRQAPGKGLEWVSGISWNSGSIGYADSVKGRFTISRDNSKNTLYLQMNSLRAEDTAVYYCARSGYSSSWFDPDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 78 - protein, 217 aa, synthetic construct
  REGION 1..217: note = antibody sequence
QSVLTQPPSASGTPGQRVTISCTGSSSNIGAGYDVHWYQQLPGTAPKLLIYGNSNRPSGVPDRFSGSKSGTSASLAISGLRSEDEADYYCSSYAGSNPYVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 79 - DNA, 1356 bp, synthetic construct
  misc_feature 1..1356: note = antibody sequence
  source 1..1356: mol_type = other DNA
gaagttcagctgctggaatctggcggcggactggttcaacctggcggatctctgagactgagctgtgccgccagcggcttcacctttagcagctacgagatgaactgggtccgacaggcccctggcaaaggccttgaatgggtgtccggcatcagctggaatagcggctctatcggctacgccgacagcgtgaagggcagattcaccatcagccgggacaacagcaagaacaccctgtacctgcagatgaacagcctgagagccgaggacaccgccgtgtactactgtgccagaagcggctacagcagctcttggtttgaccccgacttcgactattggggccagggcacactggtcacagtctcttcagccagcaccaagggccccagcgtgttccctctggcccctagcagcaagagcacatctggcggaacagccgccctgggctgcctcgtgaaggactactttcccgagcccgtgaccgtgtcctggaactctggcgctctgacaagcggcgtgcacacctttccagccgtgctgcagagcagcggcctgtactctctgagcagcgtcgtgacagtgcccagcagctctctgggcacccagacctacatctgcaacgtgaaccacaagcccagcaacaccaaggtggacaagaaggtggaacccaagagctgcgacaagacccacacctgtcccccttgtcctgcccccgaactgctgggaggcccttccgtgttcctgttccccccaaagcccaaggacaccctgatgatcagccggacccccgaagtgacctgcgtggtggtggatgtgtcccacgaggaccctgaagtgaagttcaattggtacgtggacggcgtggaagtgcacaacgccaagaccaagcctagagaggaacagtacaacagcacctaccgggtggtgtccgtgctgacagtgctgcaccaggactggctgaacggcaaagagtacaagtgcaaggtgtccaacaaggccctgcctgcccccatcgagaaaaccatcagcaaggccaagggccagccccgcgaaccccaggtgtacacactgcccccaagcagggacgagctgaccaagaaccaggtgtccctgacctgtctcgtgaaaggcttctacccctccgatatcgccgtggaatgggagagcaacggccagcccgagaacaactacaagaccaccccccctgtgctggacagcgacggctcattcttcctgtacagcaagctgaccgtggacaagtcccggtggcagcagggcaacgtgttcagctgcagcgtgatgcacgaggccctgcacaaccactacacccagaagtccctgagcctgagccctggc

SEQ ID NO: 80 - DNA, 651 bp, synthetic construct
  misc_feature 1..651: note = antibody sequence
  source 1..651: mol_type = other DNA
cagtctgttctgacacagcctcctagcgcctctggcacacctggacagagagtgaccatcagctgtaccggcagcagctccaatatcggagccggctatgacgtgcactggtatcagcagctgcctggcacagcccctaaactgctgatctacggcaacagcaacagacccagcggcgtgcccgatagattttccggctctaagagcggcacaagcgccagcctggctatctctggactgagatctgaggacgaggccgactactactgcagcagctatgccggcagcaacccctacgttgtgtttggcggaggcaccaagctgaccgttctaggccagcctaaagccgcccctagcgtgaccctgttccctccaagcagcgaggaactgcaggccaacaaggccaccctcgtgtgcctgatcagcgacttctatcctggcgccgtgaccgtggcctggaaggccgatagctctcctgtgaaggccggcgtggaaaccaccacccctagcaagcagagcaacaacaaatacgccgccagcagctacctgagcctgacccccgagcagtggaagtcccacagatcctacagctgccaagtgacccacgagggcagcaccgtggaaaagacagtggcccctaccgagtgcagc

SEQ ID NO: 81 - protein, 116 aa, synthetic construct
  REGION 1..116: note = antibody sequence
EVQLLESGGGLVQTGGSLRLSCAASGFTFSDYAMSWVRQAPGKGLEWVSWIYYDSGSKYYADSVKGRFTISRDNSKNTLYLQMNSLRAEDTAVYYCAKLNGDFDYWGQGTLVTVSS

SEQ ID NO: 82 - protein, 5 aa, synthetic construct
  REGION 1..5: note = antibody sequence
DYAMS

SEQ ID NO: 83 - protein, 17 aa, synthetic construct
  REGION 1..17: note = antibody sequence
WIYYDSGSKYYADSVKG

SEQ ID NO: 84 - protein, 7 aa, synthetic construct
  REGION 1..7: note = antibody sequence
LNGDFDY

SEQ ID NO: 85 - protein, 110 aa, synthetic construct
  REGION 1..110: note = antibody sequence
QSVLTQPPSASGTPGQRVTISCSGSSSNIGNNDVSWYQQLPGTAPKLLIYADSHRPSGVPDRFSGSKSGTSASLAISGLRSEDEADYYCGAWDSSLSGYVFGGGTKLTVL

SEQ ID NO: 86 - protein, 13 aa, synthetic construct
  REGION 1..13: note = antibody sequence
SGSSSNIGNNDVS

SEQ ID NO: 87 - protein, 7 aa, synthetic construct
  REGION 1..7: note = antibody sequence
ADSHRPS

SEQ ID NO: 88 - protein, 11 aa, synthetic construct
  REGION 1..11: note = antibody sequence
GAWDSSLSGYV

SEQ ID NO: 89 - DNA, 348 bp, synthetic construct
  misc_feature 1..348: note = antibody sequence
  source 1..348: mol_type = other DNA
gaagttcagctgctggaatctggcggcggactggttcaaacaggcggctctctgagactgagctgtgccgcctctggcttcaccttcagcgattacgccatgagctgggtccgacaggcccctggaaaaggccttgaatgggtgtcctggatctactacgacagcggcagcaagtactacgccgacagcgtgaagggcagattcaccatcagccgggacaacagcaagaacaccctgtacctgcagatgaacagcctgagagccgaggacaccgccgtgtactattgcgccaagctgaacggcgacttcgactattggggccagggcacactggtcacagtctcttca

SEQ ID NO: 90 - DNA, 15 bp, synthetic construct
  misc_feature 1..15: note = antibody sequence
  source 1..15: mol_type = other DNA
gattacgccatgagc

SEQ ID NO: 91 - DNA, 51 bp, synthetic construct
  misc_feature 1..51: note = antibody sequence
  source 1..51: mol_type = other DNA
tggatctactacgacagcggcagcaagtactacgccgacagcgtgaagggc

SEQ ID NO: 92 - DNA, 21 bp, synthetic construct
  misc_feature 1..21: note = antibody sequence
  source 1..21: mol_type = other DNA
ctgaacggcgacttcgactat

SEQ ID NO: 93 - DNA, 330 bp, synthetic construct
  misc_feature 1..330: note = antibody sequence
  source 1..330: mol_type = other DNA
cagtctgttctgacacagcctcctagcgcctctggcacacctggacagagagtgaccatcagctgtagcggcagcagctccaacatcggcaacaacgacgtgtcctggtatcagcagctgcctggcacagcccctaaactgctgatctacgccgacagccacagacctagcggcgtgccagatagattcagcggctctaagagcggcacatctgccagcctggccatctctggactgagatctgaggacgaggccgactactattgcggcgcctgggattctagcctgagcggctatgtttttggcggaggcaccaagctgaccgtgcta

SEQ ID NO: 94 - DNA, 39 bp, synthetic construct
  misc_feature 1..39: note = antibody sequence
  source 1..39: mol_type = other DNA
agcggcagcagctccaacatcggcaacaacgacgtgtcc

SEQ ID NO: 95 - DNA, 21 bp, synthetic construct
  misc_feature 1..21: note = antibody sequence
  source 1..21: mol_type = other DNA
gccgacagccacagacctagc

SEQ ID NO: 96 - DNA, 33 bp, synthetic construct
  misc_feature 1..33: note = antibody sequence
  source 1..33: mol_type = other DNA
ggcgcctgggattctagcctgagcggctatgtt

SEQ ID NO: 97 - protein, 445 aa, synthetic construct
  REGION 1..445: note = antibody sequence
EVQLLESGGGLVQTGGSLRLSCAASGFTFSDYAMSWVRQAPGKGLEWVSWIYYDSGSKYYADSVKGRFTISRDNSKNTLYLQMNSLRAEDTAVYYCAKLNGDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 98 - protein, 216 aa, synthetic construct
  REGION 1..216: note = antibody sequence
QSVLTQPPSASGTPGQRVTISCSGSSSNIGNNDVSWYQQLPGTAPKLLIYADSHRPSGVPDRFSGSKSGTSASLAISGLRSEDEADYYCGAWDSSLSGYVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 99 - DNA, 1335 bp, synthetic construct
  misc_feature 1..1335: note = antibody sequence
  source 1..1335: mol_type = other DNA
gaagttcagctgctggaatctggcggcggactggttcaaacaggcggctctctgagactgagctgtgccgcctctggcttcaccttcagcgattacgccatgagctgggtccgacaggcccctggaaaaggccttgaatgggtgtcctggatctactacgacagcggcagcaagtactacgccgacagcgtgaagggcagattcaccatcagccgggacaacagcaagaacaccctgtacctgcagatgaacagcctgagagccgaggacaccgccgtgtactattgcgccaagctgaacggcgacttcgactattggggccagggcacactggtcacagtctcttcagccagcaccaagggccccagcgtgttccctctggcccctagcagcaagagcacatctggcggaacagccgccctgggctgcctcgtgaaggactactttcccgagcccgtgaccgtgtcctggaactctggcgctctgacaagcggcgtgcacacctttccagccgtgctgcagagcagcggcctgtactctctgagcagcgtcgtgacagtgcccagcagctctctgggcacccagacctacatctgcaacgtgaaccacaagcccagcaacaccaaggtggacaagaaggtggaacccaagagctgcgacaagacccacacctgtcccccttgtcctgcccccgaactgctgggaggcccttccgtgttcctgttccccccaaagcccaaggacaccctgatgatcagccggacccccgaagtgacctgcgtggtggtggatgtgtcccacgaggaccctgaagtgaagttcaattggtacgtggacggcgtggaagtgcacaacgccaagaccaagcctagagaggaacagtacaacagcacctaccgggtggtgtccgtgctgacagtgctgcaccaggactggctgaacggcaaagagtacaagtgcaaggtgtccaacaaggccctgcctgcccccatcgagaaaaccatcagcaaggccaagggccagccccgcgaaccccaggtgtacacactgcccccaagcagggacgagctgaccaagaaccaggtgtccctgacctgtctcgtgaaaggcttctacccctccgatatcgccgtggaatgggagagcaacggccagcccgagaacaactacaagaccaccccccctgtgctggacagcgacggctcattcttcctgtacagcaagctgaccgtggacaagtcccggtggcagcagggcaacgtgttcagctgcagcgtgatgcacgaggccctgcacaaccactacacccagaagtccctgagcctgagccctggc

SEQ ID NO: 100 - DNA, 648 bp, synthetic construct
  misc_feature 1..648: note = antibody sequence
  source 1..648: mol_type = other DNA
cagtctgttctgacacagcctcctagcgcctctggcacacctggacagagagtgaccatcagctgtagcggcagcagctccaacatcggcaacaacgacgtgtcctggtatcagcagctgcctggcacagcccctaaactgctgatctacgccgacagccacagacctagcggcgtgccagatagattcagcggctctaagagcggcacatctgccagcctggccatctctggactgagatctgaggacgaggccgactactattgcggcgcctgggattctagcctgagcggctatgtttttggcggaggcaccaagctgaccgtgctaggccagcctaaagccgcccctagcgtgaccctgttccctccaagcagcgaggaactgcaggccaacaaggccaccctcgtgtgcctgatcagcgacttctatcctggcgccgtgaccgtggcctggaaggccgatagctctcctgtgaaggccggcgtggaaaccaccacccctagcaagcagagcaacaacaaatacgccgccagcagctacctgagcctgacccccgagcagtggaagtcccacagatcctacagctgccaagtgacccacgagggcagcaccgtggaaaagacagtggcccctaccgagtgcagc

SEQ ID NO: 101 - protein, 123 aa, synthetic construct; identical to SEQ ID NO: 61
  REGION 1..123: note = antibody sequence

SEQ ID NO: 102 - protein, 5 aa, synthetic construct
  REGION 1..5: note = antibody sequence
SYEMN

SEQ ID NO: 103 - protein, 17 aa, synthetic construct
  REGION 1..17: note = antibody sequence
GISWNSGSIGYADSVKG

SEQ ID NO: 104 - protein, 14 aa, synthetic construct
  REGION 1..14: note = antibody sequence
SGYSSSWFDPDFDY

SEQ ID NO: 105 - protein, 111 aa, synthetic construct
  REGION 1..111: note = antibody sequence
QSVLTQPPSVSGAPGQRVTISCTGSSSNIGAGYDVHWYQQLPGTAPKLLIYGNSNRPSGVPDRFSGSKSGTSASLAITGLQAEDEADYYCSSYAGSNPYVVFGGGTKLTVL

SEQ ID NO: 106 - protein, 14 aa, synthetic construct
  REGION 1..14: note = antibody sequence
TGSSSNIGAGYDVH

SEQ ID NO: 107 - protein, 7 aa, synthetic construct
  REGION 1..7: note = antibody sequence
GNSNRPS

SEQ ID NO: 108 - protein, 11 aa, synthetic construct
  REGION 1..11: note = antibody sequence
SSYAGSNPYVV

SEQ ID NO: 109 - DNA, 369 bp, synthetic construct; identical to SEQ ID NO: 69
  misc_feature 1..369: note = antibody sequence
  source 1..369: mol_type = other DNA

SEQ ID NO: 110 - DNA, 15 bp, synthetic construct
  misc_feature 1..15: note = antibody sequence
  source 1..15: mol_type = other DNA
agctacgagatgaac

SEQ ID NO: 111 - DNA, 51 bp, synthetic construct; identical to SEQ ID NO: 71
  misc_feature 1..51: note = antibody sequence
  source 1..51: mol_type = other DNA

SEQ ID NO: 112 - DNA, 42 bp, synthetic construct; identical to SEQ ID NO: 72
  misc_feature 1..42: note = antibody sequence
  source 1..42: mol_type = other DNA

SEQ ID NO: 113 - DNA, 333 bp, synthetic construct
  misc_feature 1..333: note = antibody sequence
  source 1..333: mol_type = other DNA
cagtctgttctgacacagcctccatctgtgtctggcgcccctggacagagagtgaccatcagctgtacaggcagcagctccaatatcggagccggctatgacgtgcactggtatcagcagctgcctggcacagcccctaaactgctgatctacggcaacagcaacagacccagcggcgtgcccgatagattttccggctctaagagcggcacaagcgccagcctggctattactggactgcaggccgaggacgaggccgactactactgttctagctacgccggcagcaacccctacgtggtgtttggcggaggcaccaagctgacagttcta

SEQ ID NO: 114 - DNA, 42 bp, synthetic construct
  misc_feature 1..42: note = antibody sequence
  source 1..42: mol_type = other DNA
acaggcagcagctccaatatcggagccggctatgacgtgcac

SEQ ID NO: 115 - DNA, 21 bp, synthetic construct
  misc_feature 1..21: note = antibody sequence
  source 1..21: mol_type = other DNA
ggcaacagcaacagacccagc

SEQ ID NO: 116 - DNA, 33 bp, synthetic construct
  misc_feature 1..33: note = antibody sequence
  source 1..33: mol_type = other DNA
tctagctacgccggcagcaacccctacgtggtg

SEQ ID NO: 117 - protein, 452 aa, synthetic construct; identical to SEQ ID NO: 77
  REGION 1..452: note = antibody sequence

SEQ ID NO: 118 - protein, 217 aa, synthetic construct
  REGION 1..217: note = antibody sequence
QSVLTQPPSVSGAPGQRVTISCTGSSSNIGAGYDVHWYQQLPGTAPKLLIYGNSNRPSGVPDRFSGSKSGTSASLAITGLQAEDEADYYCSSYAGSNPYVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 119 - DNA, 1356 bp, synthetic construct; identical to SEQ ID NO: 79
  misc_feature 1..1356: note = antibody sequence
  source 1..1356: mol_type = other DNA

SEQ ID NO: 120 - DNA, 651 bp, synthetic construct
  misc_feature 1..651: note = antibody sequence
  source 1..651: mol_type = other DNA
cagtctgttctgacacagcctccatctgtgtctggcgcccctggacagagagtgaccatcagctgtacaggcagcagctccaatatcggagccggctatgacgtgcactggtatcagcagctgcctggcacagcccctaaactgctgatctacggcaacagcaacagacccagcggcgtgcccgatagattttccggctctaagagcggcacaagcgccagcctggctattactggactgcaggccgaggacgaggccgactactactgttctagctacgccggcagcaacccctacgtggtgtttggcggaggcaccaagctgacagttctaggccagcctaaagccgcccctagcgtgaccctgttccctccaagcagcgaggaactgcaggccaacaaggccaccctcgtgtgcctgatcagcgacttctatcctggcgccgtgaccgtggcctggaaggccgatagctctcctgtgaaggccggcgtggaaaccaccacccctagcaagcagagcaacaacaaatacgccgccagcagctacctgagcctgacccccgagcagtggaagtcccacagatcctacagctgccaagtgacccacgagggcagcaccgtggaaaagacagtggcccctaccgagtgcagc

SEQ ID NO: 121 - protein, 120 aa, synthetic construct
  REGION 1..120: note = antibody sequence
EVQLLESGGGLVQPGGSLRLSCAASGFTFSSYAMSWVRQAPGKGLEWVSAIGTGGDTYYADSVKGRFTISRDNSKNTLYLQMNSLRAEDTAVYYCARRDDYTSRDAFDYWGQGTLVTVSS

SEQ ID NO: 122 - protein, 5 aa, synthetic construct
  REGION 1..5: note = antibody sequence
SYAMS

SEQ ID NO: 123 - protein, 16 aa, synthetic construct
  REGION 1..16: note = antibody sequence
AIGTGGDTYYADSVKG

SEQ ID NO: 124 - protein, 12 aa, synthetic construct
  REGION 1..12: note = antibody sequence
RDDYTSRDAFDY

SEQ ID NO: 125 - protein, 111 aa, synthetic construct
  REGION 1..111: note = antibody sequence
QSVLTQPPSASGTPGQRVTISCSGSSSNIGSNTVNWYQQLPGTAPKLLIYYDDLRPSGVPDRFSGSKSGTSASLAISGLQSEDEADYYCAAWDDSLNGYVVFGGGTKLTVL

SEQ ID NO: 126 - protein, 13 aa, synthetic construct
  REGION 1..13: note = antibody sequence
SGSSSNIGSNTVN

SEQ ID NO: 127 - protein, 7 aa, synthetic construct
  REGION 1..7: note = antibody sequence
YDDLRPS

SEQ ID NO: 128 - protein, 12 aa, synthetic construct
  REGION 1..12: note = antibody sequence
AAWDDSLNGYVV

SEQ ID NO: 129 - DNA, 360 bp, synthetic construct
  misc_feature 1..360: note = antibody sequence
  source 1..360: mol_type = other DNA
gaagttcagctgctggaatctggcggcggactggttcaacctggcggatctctgagactgagctgtgccgccagcggcttcacctttagcagctacgccatgagctgggtccgacaggctcctggcaaaggccttgaatgggtgtccgccattggcacaggcggcgatacctactacgccgactctgtgaagggcagattcaccatcagccgggacaacagcaagaacaccctgtacctgcagatgaacagcctgagagccgaggacaccgccgtgtactattgcgccagaagggacgactacaccagcagggacgccttcgattattggggccagggcacactggtcaccgtttcttca

SEQ ID NO: 130 - DNA, 15 bp, synthetic construct
  misc_feature 1..15: note = antibody sequence
  source 1..15: mol_type = other DNA
agctacgccatgagc

SEQ ID NO: 131 - DNA, 48 bp, synthetic construct
  misc_feature 1..48: note = antibody sequence
  source 1..48: mol_type = other DNA
gccattggcacaggcggcgatacctactacgccgactctgtgaagggc

SEQ ID NO: 132 - DNA, 36 bp, synthetic construct
  misc_feature 1..36: note = antibody sequence
  source 1..36: mol_type = other DNA
agggacgactacaccagcagggacgccttcgattat

SEQ ID NO: 133 - DNA, 333 bp, synthetic construct
  misc_feature 1..333: note = antibody sequence
  source 1..333: mol_type = other DNA
cagtctgttctgacacagcctcctagcgcctctggcacacctggacagagagtgaccatcagctgtagcggcagcagctccaacatcggcagcaacaccgtgaactggtatcagcagctgcctggcacagcccctaaactgctgatctactacgacgacctgcggcctagcggcgtgccagatagattttctggcagcaagagcggcacctctgccagcctggctatttctggactgcagagcgaggacgaggccgactattattgtgccgcctgggacgacagcctgaacggctatgttgttttcggcggaggcaccaagctgaccgttcta

SEQ ID NO: 134 - DNA, 39 bp, synthetic construct; identical to SEQ ID NO: 54
  misc_feature 1..39: note = antibody sequence
  source 1..39: mol_type = other DNA

SEQ ID NO: 135 - DNA, 21 bp, synthetic construct
  misc_feature 1..21: note = antibody sequence
  source 1..21: mol_type = other DNA
tacgacgacctgcggcctagc

SEQ ID NO: 136 - DNA, 36 bp, synthetic construct
  misc_feature 1..36: note = antibody sequence
  source 1..36: mol_type = other DNA
gccgcctgggacgacagcctgaacggctatgttgtt

SEQ ID NO: 137 - protein, 449 aa, synthetic construct
  REGION 1..449: note = antibody sequence
EVQLLESGGGLVQPGGSLRLSCAASGFTFSSYAMSWVRQAPGKGLEWVSAIGTGGDTYYADSVKGRFTISRDNSKNTLYLQMNSLRAEDTAVYYCARRDDYTSRDAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 138 - protein, 217 aa, synthetic construct
  REGION 1..217: note = antibody sequence
QSVLTQPPSASGTPGQRVTISCSGSSSNIGSNTVNWYQQLPGTAPKLLIYYDDLRPSGVPDRFSGSKSGTSASLAISGLQSEDEADYYCAAWDDSLNGYVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 139 - DNA, 1347 bp, synthetic construct
  misc_feature 1..1347: note = antibody sequence
  source 1..1347: mol_type = other DNA
gaagttcagctgctggaatctggcggcggactggttcaacctggcggatctctgagactgagctgtgccgccagcggcttcacctttagcagctacgccatgagctgggtccgacaggctcctggcaaaggccttgaatgggtgtccgccattggcacaggcggcgatacctactacgccgactctgtgaagggcagattcaccatcagccgggacaacagcaagaacaccctgtacctgcagatgaacagcctgagagccgaggacaccgccgtgtactattgcgccagaagggacgactacaccagcagggacgccttcgattattggggccagggcacactggtcaccgtttcttcagccagcaccaagggccccagcgtgttccctctggcccctagcagcaagagcacatctggcggaacagccgccctgggctgcctcgtgaaggactactttcccgagcccgtgaccgtgtcctggaactctggcgctctgacaagcggcgtgcacacctttccagccgtgctgcagagcagcggcctgtactctctgagcagcgtcgtgacagtgcccagcagctctctgggcacccagacctacatctgcaacgtgaaccacaagcccagcaacaccaaggtggacaagaaggtggaacccaagagctgcgacaagacccacacctgtcccccttgtcctgcccccgaactgctgggaggcccttccgtgttcctgttccccccaaagcccaaggacaccctgatgatcagccggacccccgaagtgacctgcgtggtggtggatgtgtcccacgaggaccctgaagtgaagttcaattggtacgtggacggcgtggaagtgcacaacgccaagaccaagcctagagaggaacagtacaacagcacctaccgggtggtgtccgtgctgacagtgctgcaccaggactggctgaacggcaaagagtacaagtgcaaggtgtccaacaaggccctgcctgcccccatcgagaaaaccatcagcaaggccaagggccagccccgcgaaccccaggtgtacacactgcccccaagcagggacgagctgaccaagaaccaggtgtccctgacctgtctcgtgaaaggcttctacccctccgatatcgccgtggaatgggagagcaacggccagcccgagaacaactacaagaccaccccccctgtgctggacagcgacggctcattcttcctgtacagcaagctgaccgtggacaagtcccggtggcagcagggcaacgtgttcagctgcagcgtgatgcacgaggccctgcacaaccactacacccagaagtccctgagcctgagccctggc

SEQ ID NO: 140 - DNA, 651 bp, synthetic construct
  misc_feature 1..651: note = antibody sequence
  source 1..651: mol_type = other DNA
cagtctgttctgacacagcctcctagcgcctctggcacacctggacagagagtgaccatcagctgtagcggcagcagctccaacatcggcagcaacaccgtgaactggtatcagcagctgcctggcacagcccctaaactgctgatctactacgacgacctgcggcctagcggcgtgccagatagattttctggcagcaagagcggcacctctgccagcctggctatttctggactgcagagcgaggacgaggccgactattattgtgccgcctgggacgacagcctgaacggctatgttgttttcggcggaggcaccaagctgaccgttctaggccagcctaaagccgcccctagcgtgaccctgttccctccaagcagcgaggaactgcaggccaacaaggccaccctcgtgtgcctgatcagcgacttctatcctggcgccgtgaccgtggcctggaaggccgatagctctcctgtgaaggccggcgtggaaaccaccacccctagcaagcagagcaacaacaaatacgccgccagcagctacctgagcctgacccccgagcagtggaagtcccacagatcctacagctgccaagtgacccacgagggcagcaccgtggaaaagacagtggcccctaccgagtgcagc

SEQ ID NO: 141 - protein, 120 aa, synthetic construct
  REGION 1..120: note = antibody sequence
EVQLLESGGGLVQPGGSLRLSCAASGFTFYSYAMSWVRQAPGKGLEWVSAIGTGGDTYYADSVKGRFTISRDNSKNTLYLQMNSLRAEDTAVYYCARRDDYTSRDAFDYWGQGTLVTVSS

SEQ ID NO: 142 - protein, 5 aa, synthetic construct
  REGION 1..5: note = antibody sequence
SYAMS

SEQ ID NO: 143 - protein, 16 aa, synthetic construct
  REGION 1..16: note = antibody sequence
AIGTGGDTYYADSVKG

SEQ ID NO: 144 - protein, 12 aa, synthetic construct
  REGION 1..12: note = antibody sequence
RDDYTSRDAFDY

SEQ ID NO: 145 - protein, 111 aa, synthetic construct
  REGION 1..111: note = antibody sequence
QSVLTQPPSASGTPGQRVTISCSGSSSNIGSNTVNWYQQLPGTAPKLLIYYDDLRPSGVPDRFSGSKSGTSASLAISGLQSEDEADYYCAAWDDSLNDYVVFGGGTKLTVL

SEQ ID NO: 146 - protein, 13 aa, synthetic construct
  REGION 1..13: note = antibody sequence
SGSSSNIGSNTVN

SEQ ID NO: 147 - protein, 7 aa, synthetic construct
  REGION 1..7: note = antibody sequence
YDDLRPS

SEQ ID NO: 148 - protein, 12 aa, synthetic construct
  REGION 1..12: note = antibody sequence
AAWDDSLNDYVV

SEQ ID NO: 149 - DNA, 360 bp, synthetic construct
  misc_feature 1..360: note = antibody sequence
  source 1..360: mol_type = other DNA
gaagttcagctgctggaatctggcggcggactggttcaacctggcggatctctgagactgagctgtgccgccagcggcttcaccttttacagctacgccatgagctgggtccgacaggcccctggaaaaggccttgaatgggtgtccgccatcggcacaggcggcgatacctactatgccgactctgtgaagggcagattcaccatcagccgggacaacagcaagaacaccctgtacctgcagatgaacagcctgagagccgaggacaccgccgtgtactattgcgccagaagggacgactacaccagcagggacgccttcgattattggggccagggcacactggtcaccgtttcttca

SEQ ID NO: 150 - DNA, 15 bp, synthetic construct
  misc_feature 1..15: note = antibody sequence
  source 1..15: mol_type = other DNA
agctacgccatgagc

SEQ ID NO: 151 - DNA, 48 bp, synthetic construct
  misc_feature 1..48: note = antibody sequence
  source 1..48: mol_type = other DNA
gccatcggcacaggcggcgatacctactatgccgactctgtgaagggc

SEQ ID NO: 152 - DNA, 36 bp, synthetic construct; identical to SEQ ID NO: 132
  misc_feature 1..36: note = antibody sequence
  source 1..36: mol_type = other DNA

SEQ ID NO: 153 - DNA, 333 bp, synthetic construct
  misc_feature 1..333: note = antibody sequence
  source 1..333: mol_type = other DNA
cagtctgttctgacacagcctcctagcgcctctggcacacctggacagagagtgaccatcagctgtagcggcagcagctccaacatcggcagcaacaccgtgaactggtatcagcagctgcctggcacagcccctaaactgctgatctactacgacgacctgcggcctagcggcgtgccagatagattttctggcagcaagagcggcacctctgccagcctggctatttctggactgcagagcgaggacgaggccgactattattgtgccgcctgggacgacagcctgaacgactacgttgtgtttggcggaggcaccaagctgaccgttcta

SEQ ID NO: 154 - DNA, 39 bp, synthetic construct; identical to SEQ ID NO: 54
  misc_feature 1..39: note = antibody sequence
  source 1..39: mol_type = other DNA

SEQ ID NO: 155 - DNA, 21 bp, synthetic construct
  misc_feature 1..21: note = antibody sequence
  source 1..21: mol_type = other DNA
tacgacgacctgcggcctagc

SEQ ID NO: 156 - DNA, 36 bp, synthetic construct
  misc_feature 1..36: note = antibody sequence
  source 1..36: mol_type = other DNA
gccgcctgggacgacagcctgaacgactacgttgtg

SEQ ID NO: 157 - protein, 447 aa, synthetic construct
  REGION 1..447: note = antibody sequence
EVQLLESGGGLVQPGGSLRLSCAASGFTFYSYAMSWVRQAPGKGLEWVSAIGTGGDTYYADSVKGRFTISRDNSKNTLYLQMNSLRAEDTAVYYCARRDDYTSRDAFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 158 - protein, 217 aa, synthetic construct
  REGION 1..217: note = antibody sequence
QSVLTQPPSASGTPGQRVTISCSGSSSNIGSNTVNWYQQLPGTAPKLLIYYDDLRPSGVPDRFSGSKSGTSASLAISGLQSEDEADYYCAAWDDSLNDYVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 159 - DNA, 1341 bp, synthetic construct
  misc_feature 1..1341: note = antibody sequence
  source 1..1341: mol_type = other DNA
gaagttcagctgctggaatctggcggcggactggttcaacctggcggatctctgagactgagctgtgccgccagcggcttcaccttttacagctacgccatgagctgggtccgacaggcccctggaaaaggccttgaatgggtgtccgccatcggcacaggcggcgatacctactatgccgactctgtgaagggcagattcaccatcagccgggacaacagcaagaacaccctgtacctgcagatgaacagcctgagagccgaggacaccgccgtgtactattgcgccagaagggacgactacaccagcagggacgccttcgattattggggccagggcacactggtcaccgtttcttcagccagcaccaagggccccagcgtgttccctctggccccttgtagcagaagcaccagcgagtctacagccgccctgggctgcctcgtgaaggactactttcccgagcccgtgaccgtgtcctggaactctggcgctctgacaagcggcgtgcacacctttccagccgtgctgcagagcagcggcctgtactctctgagcagcgtcgtgacagtgcccagcagcagcctgggcaccaagacctacacctgtaacgtggaccacaagcccagcaacaccaaggtggacaagcgggtggaatctaagtacggccctccctgccctccttgcccagcccctgaatttctgggcggaccctccgtgttcctgttccccccaaagcccaaggacaccctgatgatcagccggacccccgaagtgacctgcgtggtggtggatgtgtcccaggaagatcccgaggtgcagttcaattggtacgtggacggcgtggaagtgcacaacgccaagaccaagcccagagaggaacagttcaacagcacctaccgggtggtgtccgtgctgacagtgctgcaccaggactggctgaacggcaaagagtacaagtgcaaggtgtccaacaagggcctgcccagctccatcgagaaaaccatcagcaaggccaagggccagccccgcgaaccccaggtgtacacactgcctccaagccaggaagagatgaccaagaaccaggtgtccctgacctgtctcgtgaaaggcttctacccctccgatatcgccgtggaatgggagagcaacggccagcccgagaacaactacaagaccaccccccctgtgctggacagcgacggctcattcttcctgtacagcagactgaccgtggacaagagccggtggcaggaaggcaacgtgttcagctgcagcgtgatgcacgaggccctgcacaaccactacacccagaagtccctgtctctgagcctgggcaag

SEQ ID NO: 160 - DNA, 651 bp, synthetic construct
  misc_feature 1..651: note = antibody sequence
  source 1..651: mol_type = other DNA
cagtctgttctgacacagcctcctagcgcctctggcacacctggacagagagtgaccatcagctgtagcggcagcagctccaacatcggcagcaacaccgtgaactggtatcagcagctgcctggcacagcccctaaactgctgatctactacgacgacctgcggcctagcggcgtgccagatagattttctggcagcaagagcggcacctctgccagcctggctatttctggactgcagagcgaggacgaggccgactattattgtgccgcctgggacgacagcctgaacgactacgttgtgtttggcggaggcaccaagctgaccgttctaggccagcctaaagccgcccctagcgtgaccctgttccctccaagcagcgaggaactgcaggccaacaaggccaccctcgtgtgcctgatcagcgacttctatcctggcgccgtgaccgtggcctggaaggccgatagctctcctgtgaaggccggcgtggaaaccaccacccctagcaagcagagcaacaacaaatacgccgccagcagctacctgagcctgacccccgagcagtggaagtcccacagatcctacagctgccaagtgacccacgagggcagcaccgtggaaaagacagtggcccctaccgagtgcagc

SEQ ID NO: 161 - protein, 120 aa, synthetic construct
  REGION 1..120: note = antibody sequence
EVQLLESGGGLVQPGGSLRLSCAASGFTFSSYAMSWVRQAPGKGLEWVSAIGYGGDTYYADSVKGRFTISRDNSKNTLYLQMNSLRAEDTAVYYCARRDDYTSRDAFDYWGQGTLVTVSS

SEQ ID NO: 162 - protein, 5 aa, synthetic construct
  REGION 1..5: note = antibody sequence
SYAMS

SEQ ID NO: 163 - protein, 16 aa, synthetic construct
  REGION 1..16: note = antibody sequence
AIGYGGDTYYADSVKG

SEQ ID NO: 164 - protein, 12 aa, synthetic construct
  REGION 1..12: note = antibody sequence
RDDYTSRDAFDY

SEQ ID NO: 165 - protein, 111 aa, synthetic construct
  REGION 1..111: note = antibody sequence
QSVLTQPPSASGTPGQRVTISCSGSSSNIGSNTVNWYQQLPGTAPKLLIYYDDLRPSGVPDRFSGSKSGTSASLAISGLQSEDEADYYCAAWDDSLNDIVVFGGGTKLTVL

SEQ ID NO: 166 - protein, 13 aa, synthetic construct
  REGION 1..13: note = antibody sequence
SGSSSNIGSNTVN

SEQ ID NO: 167 - protein, 7 aa, synthetic construct
  REGION 1..7: note = antibody sequence
YDDLRPS

SEQ ID NO: 168 - protein, 12 aa, synthetic construct
  REGION 1..12: note = antibody sequence
AAWDDSLNDIVV

SEQ ID NO: 169 - DNA, 360 bp, synthetic construct
  misc_feature 1..360: note = antibody sequence
  source 1..360: mol_type = other DNA
gaagttcagctgctggaatctggcggcggactggttcaacctggcggatctctgagactgagctgtgccgccagcggcttcacctttagcagctacgccatgagctgggtccgacaggctcctggcaaaggccttgaatgggtgtccgccatcggctatggcggcgatacctactacgccgactctgtgaagggcagattcaccatcagccgggacaacagcaagaacaccctgtacctgcagatgaacagcctgagagccgaggacaccgccgtgtactattgcgccagaagggacgactacaccagcagggacgccttcgattattggggccagggcacactggtcaccgtttcttca

SEQ ID NO: 170 - DNA, 15 bp, synthetic construct
  misc_feature 1..15: note = antibody sequence
  source 1..15: mol_type = other DNA
agctacgccatgagc

SEQ ID NO: 171 - DNA, 48 bp, synthetic construct
  misc_feature 1..48: note = antibody sequence
  source 1..48: mol_type = other DNA
gccatcggctatggcggcgatacctactacgccgactctgtgaagggc

SEQ ID NO: 172 - DNA, 36 bp, synthetic construct; identical to SEQ ID NO: 132
  misc_feature 1..36: note = antibody sequence
  source 1..36: mol_type = other DNA

SEQ ID NO: 173 - DNA, 333 bp, synthetic construct
  misc_feature 1..333: note = antibody sequence
  source 1..333: mol_type = other DNA
cagtctgttctgacacagcctcctagcgcctctggcacacctggacagagagtgaccatcagctgtagcggcagcagctccaacatcggcagcaacaccgtgaactggtatcagcagctgcctggcacagcccctaaactgctgatctactacgacgacctgcggcctagcggcgtgccagatagattttctggcagcaagagcggcacctctgccagcctggctatttctggactgcagagcgaggacgaggccgactattattgtgccgcctgggacgacagcctgaacgacatcgttgttttcggcggaggcaccaagctgaccgttcta

SEQ ID NO: 174 - DNA, 39 bp, synthetic construct; identical to SEQ ID NO: 54
  misc_feature 1..39: note = antibody sequence
  source 1..39: mol_type = other DNA

SEQ ID NO: 175 - DNA, 21 bp, synthetic construct
  misc_feature 1..21: note = antibody sequence
  source 1..21: mol_type = other DNA
tacgacgacctgcggcctagc

SEQ ID NO: 176 - DNA, 36 bp, synthetic construct
  misc_feature 1..36: note = antibody sequence
  source 1..36: mol_type = other DNA
gccgcctgggacgacagcctgaacgacatcgttgtt

SEQ ID NO: 177 - protein, 447 aa, synthetic construct
  REGION 1..447: note = antibody sequence
EVQLLESGGGLVQPGGSLRLSCAASGFTFSSYAMSWVRQAPGKGLEWVSAIGYGGDTYYADSVKGRFTISRDNSKNTLYLQMNSLRAEDTAVYYCARRDDYTSRDAFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 178 - protein, 217 aa, synthetic construct
  REGION 1..217: note = antibody sequence
QSVLTQPPSASGTPGQRVTISCSGSSSNIGSNTVNWYQQLPGTAPKLLIYYDDLRPSGVPDRFSGSKSGTSASLAISGLQSEDEADYYCAAWDDSLNDIVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 179 - DNA, 1341 bp, synthetic construct
  misc_feature 1..1341: note = antibody sequence
  source 1..1341: mol_type = other DNA
gaagttcagctgctggaatctggcggcggactggttcaacctggcggatctctgagactgagctgtgccgccagcggcttcacctttagcagctacgccatgagctgggtccgacaggctcctggcaaaggccttgaatgggtgtccgccatcggctatggcggcgatacctactacgccgactctgtgaagggcagattcaccatcagccgggacaacagcaagaacaccctgtacctgcagatgaacagcctgagagccgaggacaccgccgtgtactattgcgccagaagggacgactacaccagcagggacgccttcgattattggggccagggcacactggtcaccgtttcttcagccagcaccaagggccccagcgtgttccctctggccccttgtagcagaagcaccagcgagtctacagccgccctgggctgcctcgtgaaggactactttcccgagcccgtgaccgtgtcctggaactctggcgctctgacaagcggcgtgcacacctttccagccgtgctgcagagcagcggcctgtactctctgagcagcgtcgtgacagtgcccagcagcagcctgggcaccaagacctacacctgtaacgtggaccacaagcccagcaacaccaaggtggacaagcgggtggaatctaagtacggccctccctgccctccttgcccagcccctgaatttctgggcggaccctccgtgttcctgttccccccaaagcccaaggacaccctgatgatcagccggacccccgaagtgacctgcgtggtggtggatgtgtcccaggaagatcccgaggtgcagttcaattggtacgtggacggcgtggaagtgcacaacgccaagaccaagcccagagaggaacagttcaacagcacctaccgggtggtgtccgtgctgacagtgctgcaccaggactggctgaacggcaaagagtacaagtgcaaggtgtccaacaagggcctgcccagctccatcgagaaaaccatcagcaaggccaagggccagccccgcgaaccccaggtgtacacactgcctccaagccaggaagagatgaccaagaaccaggtgtccctgacctgtctcgtgaaaggcttctacccctccgatatcgccgtggaatgggagagcaacggccagcccgagaacaactacaagaccaccccccctgtgctggacagcgacggctcattcttcctgtacagcagactgaccgtggacaagagccggtggcaggaaggcaacgtgttcagctgcagcgtgatgcacgaggccctgcacaaccactacacccagaagtccctgtctctgagcctgggcaag

SEQ ID NO: 180 - DNA, 651 bp, synthetic construct
  misc_feature 1..651: note = antibody sequence
  source 1..651: mol_type = other DNA
cagtctgttctgacacagcctcctagcgcctctggcacacctggacagagagtgaccatcagctgtagcggcagcagctccaacatcggcagcaacaccgtgaactggtatcagcagctgcctggcacagcccctaaactgctgatctactacgacgacctgcggcctagcggcgtgccagatagattttctggcagcaagagcggcacctctgccagcctggctatttctggactgcagagcgaggacgaggccgactattattgtgccgcctgggacgacagcctgaacgacatcgttgttttcggcggaggcaccaagctgaccgttctaggccagcctaaagccgcccctagcgtgaccctgttccctccaagcagcgaggaactgcaggccaacaaggccaccctcgtgtgcctgatcagcgacttctatcctggcgccgtgaccgtggcctggaaggccgatagctctcctgtgaaggccggcgtggaaaccaccacccctagcaagcagagcaacaacaaatacgccgccagcagctacctgagcctgacccccgagcagtggaagtcccacagatcctacagctgccaagtgacccacgagggcagcaccgtggaaaagacagtggcccctaccgagtgcagc

SEQ ID NO: 181 - protein, 120 aa, synthetic construct; identical to SEQ ID NO: 161
  REGION 1..120: note = antibody sequence

SEQ ID NO: 182 - protein, 5 aa, synthetic construct
  REGION 1..5: note = antibody sequence
SYAMS

SEQ ID NO: 183 - protein, 16 aa, synthetic construct
  REGION 1..16: note = antibody sequence
AIGYGGDTYYADSVKG

SEQ ID NO: 184 - protein, 12 aa, synthetic construct
  REGION 1..12: note = antibody sequence
RDDYTSRDAFDY

SEQ ID NO: 185 - protein, 111 aa, synthetic construct
  REGION 1..111: note = antibody sequence
QSVLTQPPSASGTPGQRVTISCSGSSSNIGSNTVNWYQQLPGTAPKLLIYYDDLRPSGVPDRFSGSKSGTSASLAISGLQSEDEADYYCAAWDDSLNVYPVFGGGTKLTVL

SEQ ID NO: 186 - protein, 13 aa, synthetic construct
  REGION 1..13: note = antibody sequence
SGSSSNIGSNTVN

SEQ ID NO: 187 - protein, 7 aa, synthetic construct
  REGION 1..7: note = antibody sequence
YDDLRPS

SEQ ID NO: 188 - protein, 12 aa, synthetic construct
  REGION 1..12: note = antibody sequence
AAWDDSLNVYPV

SEQ ID NO: 189 - DNA, 360 bp, synthetic construct; identical to SEQ ID NO: 169
  misc_feature 1..360: note = antibody sequence
  source 1..360: mol_type = other DNA

SEQ ID NO: 190 - DNA, 15 bp, synthetic construct
  misc_feature 1..15: note = antibody sequence
  source 1..15: mol_type = other DNA
agctacgccatgagc

SEQ ID NO: 191 - DNA, 48 bp, synthetic construct; identical to SEQ ID NO: 171
  misc_feature 1..48: note = antibody sequence
  source 1..48: mol_type = other DNA

SEQ ID NO: 192 - DNA, 36 bp, synthetic construct; identical to SEQ ID NO: 132
  misc_feature 1..36: note = antibody sequence
  source 1..36: mol_type = other DNA

SEQ ID NO: 193 - DNA, 333 bp, synthetic construct
  misc_feature 1..333: note = antibody sequence
  source 1..333: mol_type = other DNA
cagtctgttctgacacagcctcctagcgcctctggcacacctggacagagagtgaccatcagctgtagcggcagcagctccaacatcggcagcaacaccgtgaactggtatcagcagctgcctggcacagcccctaaactgctgatctactacgacgacctgcggcctagcggcgtgccagatagattttctggcagcaagagcggcacctctgccagcctggctatttctggactgcagagcgaggacgaggccgactattattgtgccgcctgggacgacagcctgaacgtgtaccctgtttttggcggaggcaccaagctgaccgttcta

SEQ ID NO: 194 - DNA, 39 bp, synthetic construct; identical to SEQ ID NO: 54
  misc_feature 1..39: note = antibody sequence
  source 1..39: mol_type = other DNA

SEQ ID NO: 195 - DNA, 21 bp, synthetic construct
  misc_feature 1..21: note = antibody sequence
  source 1..21: mol_type = other DNA
tacgacgacctgcggcctagc

SEQ ID NO: 196 - DNA, 36 bp, synthetic construct
  misc_feature 1..36: note = antibody sequence
  source 1..36: mol_type = other DNA
gccgcctgggacgacagcctgaacgtgtaccctgtt

SEQ ID NO: 197 - protein, 447 aa, synthetic construct; identical to SEQ ID NO: 177
  REGION 1..447: note = antibody sequence

SEQ ID NO: 198 - protein, 217 aa, synthetic construct
  REGION 1..217: note = antibody sequence
QSVLTQPPSASGTPGQRVTISCSGSSSNIGSNTVNWYQQLPGTAPKLLIYYDDLRPSGVPDRFSGSKSGTSASLAISGLQSEDEADYYCAAWDDSLNVYPVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 199 - DNA, 1341 bp, synthetic construct; identical to SEQ ID NO: 179
  misc_feature 1..1341: note = antibody sequence
  source 1..1341: mol_type = other DNA

SEQ ID NO: 200 - DNA, 651 bp, synthetic construct
  misc_feature 1..651: note = antibody sequence
  source 1..651: mol_type = other DNA
cagtctgttctgacacagcctcctagcgcctctggcacacctggacagagagtgaccatcagctgtagcggcagcagctccaacatcggcagcaacaccgtgaactggtatcagcagctgcctggcacagcccctaaactgctgatctactacgacgacctgcggcctagcggcgtgccagatagattttctggcagcaagagcggcacctctgccagcctggctatttctggactgcagagcgaggacgaggccgactattattgtgccgcctgggacgacagcctgaacgtgtaccctgtttttggcggaggcaccaagctgaccgttctaggccagcctaaagccgcccctagcgtgaccctgttccctccaagcagcgaggaactgcaggccaacaaggccaccctcgtgtgcctgatcagcgacttctatcctggcgccgtgaccgtggcctggaaggccgatagctctcctgtgaaggccggcgtggaaaccaccacccctagcaagcagagcaacaacaaatacgccgccagcagctacctgagcctgacccccgagcagtggaagtcccacagatcctacagctgccaagtgacccacgagggcagcaccgtggaaaagacagtggcccctaccgagtgcagc

SEQ ID NO: 201 - protein, 120 aa, synthetic construct; identical to SEQ ID NO: 161
  REGION 1..120: note = antibody sequence

SEQ ID NO: 202 - protein, 5 aa, synthetic construct
  REGION 1..5: note = antibody sequence
SYAMS

SEQ ID NO: 203 - protein, 16 aa, synthetic construct
  REGION 1..16: note = antibody sequence
AIGYGGDTYYADSVKG

SEQ ID NO: 204 - protein, 12 aa, synthetic construct
  REGION 1..12: note = antibody sequence
RDDYTSRDAFDY

SEQ ID NO: 205 - protein, 111 aa, synthetic construct
  REGION 1..111: note = antibody sequence
QSVLTQPPSASGTPGQRVTISCSGSSSNIGSNTVNWYQQLPGTAPKLLIYYDDLRPSGVPDRFSGSKSGTSASLAISGLQSEDEADYYCHAWDDSLNDIVVFGGGTKLTVL

SEQ ID NO: 206 - protein, 13 aa, synthetic construct
  REGION 1..13: note = antibody sequence
SGSSSNIGSNTVN

SEQ ID NO: 207 - protein, 7 aa, synthetic construct
  REGION 1..7: note = antibody sequence
YDDLRPS

SEQ ID NO: 208 - protein, 12 aa, synthetic construct
  REGION 1..12: note = antibody sequence
HAWDDSLNDIVV

SEQ ID NO: 209 - DNA, 360 bp, synthetic construct; identical to SEQ ID NO: 169
  misc_feature 1..360: note = antibody sequence
  source 1..360: mol_type = other DNA

SEQ ID NO: 210 - DNA, 15 bp, synthetic construct
  misc_feature 1..15: note = antibody sequence
  source 1..15: mol_type = other DNA
agctacgccatgagc

SEQ ID NO: 211 - DNA, 48 bp, synthetic construct; identical to SEQ ID NO: 171
  misc_feature 1..48: note = antibody sequence
  source 1..48: mol_type = other DNA

SEQ ID NO: 212 - DNA, 36 bp, synthetic construct; identical to SEQ ID NO: 132
  misc_feature 1..36: note = antibody sequence
  source 1..36: mol_type = other DNA

SEQ ID NO: 213 - DNA, 333 bp, synthetic construct
  misc_feature 1..333: note = antibody sequence
  source 1..333: mol_type = other DNA
cagtctgttctgacacagcctcctagcgcctctggcacacctggacagagagtgaccatcagctgtagcggcagcagctccaacatcggcagcaacaccgtgaactggtatcagcagctgcctggcacagcccctaaactgctgatctactacgacgacctgcggcctagcggcgtgccagatagattttctggcagcaagagcggcacctctgccagcctggctatttctggactgcagagcgaggacgaggccgactactattgtcacgcctgggacgacagcctgaacgacatcgtggtttttggcggaggcaccaagctgaccgttcta

SEQ ID NO: 214 - DNA, 39 bp, synthetic construct; identical to SEQ ID NO: 54
  misc_feature 1..39: note = antibody sequence
  source 1..39: mol_type = other DNA

SEQ ID NO: 215 - DNA, 21 bp, synthetic construct
  misc_feature 1..21: note = antibody sequence
  source 1..21: mol_type = other DNA
tacgacgacctgcggcctagc

SEQ ID NO: 216 - DNA, 36 bp, synthetic construct
  misc_feature 1..36: note = antibody sequence
  source 1..36: mol_type = other DNA
cacgcctgggacgacagcctgaacgacatcgtggtt

SEQ ID NO: 217 - protein, 447 aa, synthetic construct; identical to SEQ ID NO: 177
  REGION 1..447: note = antibody sequence

SEQ ID NO: 218 - protein, 217 aa, synthetic construct
  REGION 1..217: note = antibody sequence
QSVLTQPPSASGTPGQRVTISCSGSSSNIGSNTVNWYQQLPGTAPKLLIYYDDLRPSGVPDRFSGSKSGTSASLAISGLQSEDEADYYCHAWDDSLNDIVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 219 - DNA, 1341 bp, synthetic construct; identical to SEQ ID NO: 179
  misc_feature 1..1341: note = antibody sequence
  source 1..1341: mol_type = other DNA

SEQ ID NO: 220 - DNA, 651 bp, synthetic construct
  misc_feature 1..651: note = antibody sequence
  source 1..651: mol_type = other DNA
cagtctgttctgacacagcctcctagcgcctctggcacacctggacagagagtgaccatcagctgtagcggcagcagctccaacatcggcagcaacaccgtgaactggtatcagcagctgcctggcacagcccctaaactgctgatctactacgacgacctgcggcctagcggcgtgccagatagattttctggcagcaagagcggcacctctgccagcctggctatttctggactgcagagcgaggacgaggccgactactattgtcacgcctgggacgacagcctgaacgacatcgtggtttttggcggaggcaccaagctgaccgttctaggccagcctaaagccgcccctagcgtgaccctgttccctccaagcagcgaggaactgcaggccaacaaggccaccctcgtgtgcctgatcagcgacttctatcctggcgccgtgaccgtggcctggaaggccgatagctctcctgtgaaggccggcgtggaaaccaccacccctagcaagcagagcaacaacaaatacgccgccagcagctacctgagcctgacccccgagcagtggaagtcccacagatcctacagctgccaagtgacccacgagggcagcaccgtggaaaagacagtggcccctaccgagtgcagc

SEQ ID NO: 221 - protein, 120 aa, synthetic construct; identical to SEQ ID NO: 161
  REGION 1..120: note = antibody sequence

SEQ ID NO: 222 - protein, 5 aa, synthetic construct
  REGION 1..5: note = antibody sequence
SYAMS

SEQ ID NO: 223 - protein, 16 aa, synthetic construct
  REGION 1..16: note = antibody sequence
AIGYGGDTYYADSVKG

SEQ ID NO: 224 - protein, 12 aa, synthetic construct
  REGION 1..12: note = antibody sequence
RDDYTSRDAFDY

SEQ ID NO: 225 - protein, 111 aa, synthetic construct
  REGION 1..111: note = antibody sequence
QSVLTQPPSASGTPGQRVTISCSGSSSNIGSNTVNWYQQLPGTAPKLLIYYDDLRPSGVPDRFSGSKSGTSASLAISGLQSEDEADYYCHAWDDSLNDYPVFGGGTKLTVL

SEQ ID NO: 226 - protein, 13 aa, synthetic construct
  REGION 1..13: note = antibody sequence
SGSSSNIGSNTVN

SEQ ID NO: 227 - protein, 7 aa, synthetic construct
  REGION 1..7: note = antibody sequence
YDDLRPS

SEQ ID NO: 228 - protein, 12 aa, synthetic construct
  REGION 1..12: note = antibody sequence
HAWDDSLNDYPV

SEQ ID NO: 229 - DNA, 360 bp, synthetic construct; identical to SEQ ID NO: 169
  misc_feature 1..360: note = antibody sequence
  source 1..360: mol_type = other DNA

SEQ ID NO: 230 - DNA, 15 bp, synthetic construct
  misc_feature 1..15: note = antibody sequence
  source 1..15: mol_type = other DNA
agctacgccatgagc

SEQ ID NO: 231 - DNA, 48 bp, synthetic construct; identical to SEQ ID NO: 171
  misc_feature 1..48: note = antibody sequence
  source 1..48: mol_type = other DNA

SEQ ID NO: 232 - DNA, 36 bp, synthetic construct; identical to SEQ ID NO: 132
  misc_feature 1..36: note = antibody sequence
  source 1..36: mol_type = other DNA

SEQ ID NO: 233 - DNA, 333 bp, synthetic construct
  misc_feature 1..333: note = antibody sequence
  source 1..333: mol_type = other DNA
cagtctgttctgacacagcctcctagcgcctctggcacacctggacagagagtgaccatcagctgtagcggcagcagctccaacatcggcagcaacaccgtgaactggtatcagcagctgcctggcacagcccctaaactgctgatctactacgacgacctgcggcctagcggcgtgccagatagattttctggcagcaagagcggcacctctgccagcctggctatttctggactgcagagcgaggacgaggccgactactattgtcacgcctgggacgacagcctgaacgactaccctgtttttggcggaggcaccaagctgaccgttcta

SEQ ID NO: 234 - DNA, 39 bp, synthetic construct; identical to SEQ ID NO: 54
  misc_feature 1..39: note = antibody sequence
  source 1..39: mol_type = other DNA

SEQ ID NO: 235 - DNA, 21 bp, synthetic construct
  misc_feature 1..21: note = antibody sequence
  source 1..21: mol_type = other DNA
tacgacgacctgcggcctagc

SEQ ID NO: 236 - DNA, 36 bp, synthetic construct
  misc_feature 1..36: note = antibody sequence
  source 1..36: mol_type = other DNA
cacgcctgggacgacagcctgaacgactaccctgtt

SEQ ID NO: 237 - protein, 447 aa, synthetic construct; identical to SEQ ID NO: 177
  REGION 1..447: note = antibody sequence

SEQ ID NO: 238 - protein, 217 aa, synthetic construct
  REGION 1..217: note = antibody sequence
QSVLTQPPSASGTPGQRVTISCSGSSSNIGSNTVNWYQQLPGTAPKLLIYYDDLRPSGVPDRFSGSKSGTSASLAISGLQSEDEADYYCHAWDDSLNDYPVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 239 - DNA, 1341 bp, synthetic construct; identical to SEQ ID NO: 179
  misc_feature 1..1341: note = antibody sequence
  source 1..1341: mol_type = other DNA

SEQ ID NO: 240 - DNA, 651 bp, synthetic construct
  misc_feature 1..651: note = antibody sequence
  source 1..651: mol_type = other DNA
cagtctgttctgacacagcctcctagcgcctctggcacacctggacagagagtgaccatcagctgtagcggcagcagctccaacatcggcagcaacaccgtgaactggtatcagcagctgcctggcacagcccctaaactgctgatctactacgacgacctgcggcctagcggcgtgccagatagattttctggcagcaagagcggcacctctgccagcctggctatttctggactgcagagcgaggacgaggccgactactattgtcacgcctgggacgacagcctgaacgactaccctgtttttggcggaggcaccaagctgaccgttctaggccagcctaaagccgcccctagcgtgaccctgttccctccaagcagcgaggaactgcaggccaacaaggccaccctcgtgtgcctgatcagcgacttctatcctggcgccgtgaccgtggcctggaaggccgatagctctcctgtgaaggccggcgtggaaaccaccacccctagcaagcagagcaacaacaaatacgccgccagcagctacctgagcctgacccccgagcagtggaagtcccacagatcctacagctgccaagtgacccacgagggcagcaccgtggaaaagacagtggcccctaccgagtgcagc

SEQ ID NO: 241 - protein, 120 aa, synthetic construct; identical to SEQ ID NO: 161
  REGION 1..120: note = antibody sequence

SEQ ID NO: 242 - protein, 5 aa, synthetic construct
  REGION 1..5: note = antibody sequence
SYAMS

SEQ ID NO: 243 - protein, 16 aa, synthetic construct
  REGION 1..16: note = antibody sequence
AIGYGGDTYYADSVKG

SEQ ID NO: 244 - protein, 12 aa, synthetic construct
  REGION 1..12: note = antibody sequence
RDDYTSRDAFDY

SEQ ID NO: 245 - protein, 111 aa, synthetic construct
  REGION 1..111: note = antibody sequence
QSVLTQPPSASGTPGQRVTISCSGSSSNIGSNTVNWYQQLPGTAPKLLIYYDDLRPSGVPDRFSGSKSGTSASLAISGLQSEDEADYYCHAWDDSLNVYPVFGGGTKLTVL

SEQ ID NO: 246 - protein, 13 aa, synthetic construct
  REGION 1..13: note = antibody sequence
SGSSSNIGSNTVN

SEQ ID NO: 247 - protein, 7 aa, synthetic construct
  REGION 1..7: note = antibody sequence
YDDLRPS

SEQ ID NO: 248 - protein, 12 aa, synthetic construct
  REGION 1..12: note = antibody sequence
HAWDDSLNVYPV

SEQ ID NO: 249 - DNA, 360 bp, synthetic construct; identical to SEQ ID NO: 169
  misc_feature 1..360: note = antibody sequence
  source 1..360: mol_type = other DNA

SEQ ID NO: 250 - DNA, 15 bp, synthetic construct
  misc_feature 1..15: note = antibody sequence
  source 1..15: mol_type = other DNA
agctacgccatgagc

SEQ ID NO: 251 - DNA, 48 bp, synthetic construct; identical to SEQ ID NO: 171
  misc_feature 1..48: note = antibody sequence
  source 1..48: mol_type = other DNA

SEQ ID NO: 252 - DNA, 36 bp, synthetic construct; identical to SEQ ID NO: 132
  misc_feature 1..36: note = antibody sequence
  source 1..36: mol_type = other DNA

SEQ ID NO: 253 - DNA, 333 bp, synthetic construct
  misc_feature 1..333: note = antibody sequence
  source 1..333: mol_type = other DNA
cagtctgttctgacacagcctcctagcgcctctggcacacctggacagagagtgaccatcagctgtagcggcagcagctccaacatcggcagcaacaccgtgaactggtatcagcagctgcctggcacagcccctaaactgctgatctactacgacgacctgcggcctagcggcgtgccagatagattttctggcagcaagagcggcacctctgccagcctggctatttctggactgcagagcgaggacgaggccgactactattgtcacgcctgggacgacagcctgaacgtgtaccctgtttttggcggaggcaccaagctgaccgttcta

SEQ ID NO: 254 - DNA, 39 bp, synthetic construct; identical to SEQ ID NO: 54
  misc_feature 1..39: note = antibody sequence
  source 1..39: mol_type = other DNA

SEQ ID NO: 255 - DNA, 21 bp, synthetic construct
  misc_feature 1..21: note = antibody sequence
  source 1..21: mol_type = other DNA
tacgacgacctgcggcctagc

SEQ ID NO: 256 - DNA, 36 bp, synthetic construct
  misc_feature 1..36: note = antibody sequence
  source 1..36: mol_type = other DNA
cacgcctgggacgacagcctgaacgtgtaccctgtt

SEQ ID NO: 257 - protein, 447 aa, synthetic construct; identical to SEQ ID NO: 177
  REGION 1..447: note = antibody sequence

SEQ ID NO: 258 - protein, 217 aa, synthetic construct
  REGION 1..217: note = antibody sequence
QSVLTQPPSASGTPGQRVTISCSGSSSNIGSNTVNWYQQLPGTAPKLLIYYDDLRPSGVPDRFSGSKSGTSASLAISGLQSEDEADYYCHAWDDSLNVYPVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 259 - DNA, 1341 bp, synthetic construct; identical to SEQ ID NO: 179
  misc_feature 1..1341: note = antibody sequence
  source 1..1341: mol_type = other DNA

SEQ ID NO: 260 - DNA, 651 bp, synthetic construct
  misc_feature 1..651: note = antibody sequence
  source 1..651: mol_type = other DNA
cagtctgttctgacacagcctcctagcgcctctggcacacctggacagagagtgaccatcagctgtagcggcagcagctccaacatcggcagcaacaccgtgaactggtatcagcagctgcctggcacagcccctaaactgctgatctactacgacgacctgcggcctagcggcgtgccagatagattttctggcagcaagagcggcacctctgccagcctggctatttctggactgcagagcgaggacgaggccgactactattgtcacgcctgggacgacagcctgaacgtgtaccctgtttttggcggaggcaccaagctgaccgttctaggccagcctaaagccgcccctagcgtgaccctgttccctccaagcagcgaggaactgcaggccaacaaggccaccctcgtgtgcctgatcagcgacttctatcctggcgccgtgaccgtggcctggaaggccgatagctctcctgtgaaggccggcgtggaaaccaccacccctagcaagcagagcaacaacaaatacgccgccagcagctacctgagcctgacccccgagcagtggaagtcccacagatcctacagctgccaagtgacccacgagggcagcaccgtggaaaagacagtggcccctaccgagtgcagc

SEQ ID NO: 261 - protein, 120 aa, synthetic construct; identical to SEQ ID NO: 161
  REGION 1..120: note = antibody sequence

SEQ ID NO: 262 - protein, 5 aa, synthetic construct
  REGION 1..5: note = antibody sequence
SYAMS

SEQ ID NO: 263 - protein, 16 aa, synthetic construct
  REGION 1..16: note = antibody sequence
AIGYGGDTYYADSVKG

SEQ ID NO: 264 - protein, 12 aa, synthetic construct
  REGION 1..12: note = antibody sequence
RDDYTSRDAFDY

SEQ ID NO: 265 - protein, 111 aa, synthetic construct
  REGION 1..111: note = antibody sequence
QSVLTQPPSASGTPGQRVTISCSGSSSNIGSNTVNWYQQLPGTAPKLLIYYDDLRPSGVPDRFSGSKSGTSASLAISGLQSEDEADYYCHAWDDSLNVIPVFGGGTKLTVL

SEQ ID NO: 266 - protein, 13 aa, synthetic construct
  REGION 1..13: note = antibody sequence
SGSSSNIGSNTVN

SEQ ID NO: 267 - protein, 7 aa, synthetic construct
  REGION 1..7: note = antibody sequence
YDDLRPS

SEQ ID NO: 268 - protein, 12 aa, synthetic construct
  REGION 1..12: note = antibody sequence
HAWDDSLNVIPV

SEQ ID NO: 269 - DNA, 360 bp, synthetic construct; identical to SEQ ID NO: 169
  misc_feature 1..360: note = antibody sequence
  source 1..360: mol_type = other DNA

SEQ ID NO: 270 - DNA, 15 bp, synthetic construct
  misc_feature 1..15: note = antibody sequence
  source 1..15: mol_type = other DNA
agctacgccatgagc

SEQ ID NO: 271 - DNA, 48 bp, synthetic construct; identical to SEQ ID NO: 171
  misc_feature 1..48: note = antibody sequence
  source 1..48: mol_type = other DNA

SEQ ID NO: 272 - DNA, 36 bp, synthetic construct; identical to SEQ ID NO: 132
  misc_feature 1..36: note = antibody sequence
  source 1..36: mol_type = other DNA

SEQ ID NO: 273 - DNA, 333 bp, synthetic construct
  misc_feature 1..333: note = antibody sequence
  source 1..333: mol_type = other DNA
cagtctgttctgacacagcctcctagcgcctctggcacacctggacagagagtgaccatcagctgtagcggcagcagctccaacatcggcagcaacaccgtgaactggtatcagcagctgcctggcacagcccctaaactgctgatctactacgacgacctgcggcctagcggcgtgccagatagattttctggcagcaagagcggcacctctgccagcctggctatttctggactgcagagcgaggacgaggccgactactattgtcacgcctgggacgacagcctgaacgtgatccctgtttttggcggaggcaccaagctgaccgttcta

SEQ ID NO: 274 - DNA, 39 bp, synthetic construct; identical to SEQ ID NO: 54
  misc_feature 1..39: note = antibody sequence
  source 1..39: mol_type = other DNA

SEQ ID NO: 275 - DNA, 21 bp, synthetic construct
  misc_feature 1..21: note = antibody sequence
  source 1..21: mol_type = other DNA
tacgacgacctgcggcctagc

SEQ ID NO: 276 - DNA, 36 bp, synthetic construct
  misc_feature 1..36: note = antibody sequence
  source 1..36: mol_type = other DNA
cacgcctgggacgacagcctgaacgtgatccctgtt

SEQ ID NO: 277 - protein, 447 aa, synthetic construct; identical to SEQ ID NO: 177
  REGION 1..447: note = antibody sequence

SEQ ID NO: 278 - protein, 217 aa, synthetic construct
  REGION 1..217: note = antibody sequence
QSVLTQPPSASGTPGQRVTISCSGSSSNIGSNTVNWYQQLPGTAPKLLIYYDDLRPSGVPDRFSGSKSGTSASLAISGLQSEDEADYYCHAWDDSLNVIPVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 279 - DNA, 1341 bp, synthetic construct; identical to SEQ ID NO: 179
  misc_feature 1..1341: note = antibody sequence
  source 1..1341: mol_type = other DNA

SEQ ID NO: 280 - DNA, 651 bp, synthetic construct
  misc_feature 1..651: note = antibody sequence
  source 1..651: mol_type = other DNA
cagtctgttctgacacagcctcctagcgcctctggcacacctggacagagagtgaccatcagctgtagcggcagcagctccaacatcggcagcaacaccgtgaactggtatcagcagctgcctggcacagcccctaaactgctgatctactacgacgacctgcggcctagcggcgtgccagatagattttctggcagcaagagcggcacctctgccagcctggctatttctggactgcagagcgaggacgaggccgactactattgtcacgcctgggacgacagcctgaacgtgatccctgtttttggcggaggcaccaagctgaccgttctaggccagcctaaagccgcccctagcgtgaccctgttccctccaagcagcgaggaactgcaggccaacaaggccaccctcgtgtgcctgatcagcgacttctatcctggcgccgtgaccgtggcctggaaggccgatagctctcctgtgaaggccggcgtggaaaccaccacccctagcaagcagagcaacaacaaatacgccgccagcagctacctgagcctgacccccgagcagtggaagtcccacagatcctacagctgccaagtgacccacgagggcagcaccgtggaaaagacagtggcccctaccgagtgcagc

SEQ ID NO: 281 - protein, 120 aa, synthetic construct
  REGION 1..120: note = antibody sequence
EVQLLESGGGLVQPGGSLRLSCAASGFTFYSYAMLWVRQAPGKGLEWVSAIGTGGDTYYADSVKGRFTISRDNSKNTLYLQMNSLRAEDTAVYYCARRDDYTSRDAFDYWGQGTLVTVSS

SEQ ID NO: 282 - protein, 5 aa, synthetic construct
  REGION 1..5: note = antibody sequence
SYAML

SEQ ID NO: 283 - protein, 16 aa, synthetic construct
  REGION 1..16: note = antibody sequence
AIGTGGDTYYADSVKG

SEQ ID NO: 284 - protein, 12 aa, synthetic construct
  REGION 1..12: note = antibody sequence
RDDYTSRDAFDY

SEQ ID NO: 285 - protein, 111 aa, synthetic construct; identical to SEQ ID NO: 145
  REGION 1..111: note = antibody sequence

SEQ ID NO: 286 - protein, 13 aa, synthetic construct
  REGION 1..13: note = antibody sequence
SGSSSNIGSNTVN

SEQ ID NO: 287 - protein, 7 aa, synthetic construct
  REGION 1..7: note = antibody sequence
YDDLRPS

SEQ ID NO: 288 - protein, 12 aa, synthetic construct
  REGION 1..12: note = antibody sequence
AAWDDSLNDYVV

SEQ ID NO: 289 - DNA, 360 bp, synthetic construct
  misc_feature 1..360: note = antibody sequence
  source 1..360: mol_type = other DNA
gaagttcagctgctggaatctggcggcggactggttcaacctggcggatctctgagactgagctgtgccgccagcggcttcaccttttacagctacgccatgctgtgggtccgacaggcccctggaaaaggccttgaatgggtgtccgccatcggcacaggcggcgatacctactatgccgactctgtgaagggcagattcaccatcagccgggacaacagcaagaacaccctgtacctgcagatgaacagcctgagagccgaggacaccgccgtgtactattgcgccagaagggacgactacaccagcagggacgccttcgattattggggccagggcacactggtcaccgtttcttca

SEQ ID NO: 290 - DNA, 15 bp, synthetic construct
  misc_feature 1..15: note = antibody sequence
  source 1..15: mol_type = other DNA
agctacgccatgctg

SEQ ID NO: 291 - DNA, 48 bp, synthetic construct; identical to SEQ ID NO: 151
  misc_feature 1..48: note = antibody sequence
  source 1..48: mol_type = other DNA

SEQ ID NO: 292 - DNA, 36 bp, synthetic construct; identical to SEQ ID NO: 132
  misc_feature 1..36: note = antibody sequence
  source 1..36: mol_type = other DNA

SEQ ID NO: 293 - DNA, 333 bp, synthetic construct; identical to SEQ ID NO: 153
  misc_feature 1..333: note = antibody sequence
  source 1..333: mol_type = other DNA

SEQ ID NO: 294 - DNA, 39 bp, synthetic construct; identical to SEQ ID NO: 54
  misc_feature 1..39: note = antibody sequence
  source 1..39: mol_type = other DNA

SEQ ID NO: 295 - DNA, 21 bp, synthetic construct
  misc_feature 1..21: note = antibody sequence
  source 1..21: mol_type = other DNA
tacgacgacctgcggcctagc

SEQ ID NO: 296 - DNA, 36 bp, synthetic construct; identical to SEQ ID NO: 156
  misc_feature 1..36: note = antibody sequence
  source 1..36: mol_type = other DNA

SEQ ID NO: 297 - protein, 447 aa, synthetic construct
  REGION 1..447: note = antibody sequence
EVQLLESGGGLVQPGGSLRLSCAASGFTFYSYAMLWVRQAPGKGLEWVSAIGTGGDTYYADSVKGRFTISRDNSKNTLYLQMNSLRAEDTAVYYCARRDDYTSRDAFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 298 - protein, 217 aa, synthetic construct; identical to SEQ ID NO: 158
  REGION 1..217: note = antibody sequence

SEQ ID NO: 299 - DNA, 1341 bp, synthetic construct
  misc_feature 1..1341: note = antibody sequence
  source 1..1341: mol_type = other DNA
gaagttcagctgctggaatctggcggcggactggttcaacctggcggatctctgagactgagctgtgccgccagcggcttcaccttttacagctacgccatgctgtgggtccgacaggcccctggaaaaggccttgaatgggtgtccgccatcggcacaggcggcgatacctactatgccgactctgtgaagggcagattcaccatcagccgggacaacagcaagaacaccctgtacctgcagatgaacagcctgagagccgaggacaccgccgtgtactattgcgccagaagggacgactacaccagcagggacgccttcgattattggggccagggcacactggtcaccgtttcttcagccagcaccaagggccccagcgtgttccctctggccccttgtagcagaagcaccagcgagtctacagccgccctgggctgcctcgtgaaggactactttcccgagcccgtgaccgtgtcctggaactctggcgctctgacaagcggcgtgcacacctttccagccgtgctgcagagcagcggcctgtactctctgagcagcgtcgtgacagtgcccagcagcagcctgggcaccaagacctacacctgtaacgtggaccacaagcccagcaacaccaaggtggacaagcgggtggaatctaagtacggccctccctgccctccttgcccagcccctgaatttctgggcggaccctccgtgttcctgttccccccaaagcccaaggacaccctgatgatcagccggacccccgaagtgacctgcgtggtggtggatgtgtcccaggaagatcccgaggtgcagttcaattggtacgtggacggcgtggaagtgcacaacgccaagaccaagcccagagaggaacagttcaacagcacctaccgggtggtgtccgtgctgacagtgctgcaccaggactggctgaacggcaaagagtacaagtgcaaggtgtccaacaagggcctgcccagctccatcgagaaaaccatcagcaaggccaagggccagccccgcgaaccccaggtgtacacactgcctccaagccaggaagagatgaccaagaaccaggtgtccctgacctgtctcgtgaaaggcttctacccctccgatatcgccgtggaatgggagagcaacggccagcccgagaacaactacaagaccaccccccctgtgctggacagcgacggctcattcttcctgtacagcagactgaccgtggacaagagccggtggcaggaaggcaacgtgttcagctgcagcgtgatgcacgaggccctgcacaaccactacacccagaagtccctgtctctgagcctgggcaag

SEQ ID NO: 300 - DNA, 651 bp, synthetic construct; identical to SEQ ID NO: 160
  misc_feature 1..651: note = antibody sequence
  source 1..651: mol_type = other DNA

SEQ ID NO: 301 - protein, 120 aa, synthetic construct; identical to SEQ ID NO: 281
  REGION 1..120: note = antibody sequence

SEQ ID NO: 302 - protein, 5 aa, synthetic construct
  REGION 1..5: note = antibody sequence
SYAML

SEQ ID NO: 303 - protein, 16 aa, synthetic construct
  REGION 1..16: note = antibody sequence
AIGTGGDTYYADSVKG

SEQ ID NO: 304 - protein, 12 aa, synthetic construct
  REGION 1..12: note = antibody sequence
RDDYTSRDAFDY

SEQ ID NO: 305 - protein, 111 aa, synthetic construct
  REGION 1..111: note = antibody sequence
QSVLTQPPSASGTPGQRVTISCSGSSSNIGSNTVNWYQQLPGTAPKLLIYYDDLRPSGVPDRFSGSKSGTSASLAISGLQSEDEADYYCAAWDDSLNVYVVFGGGTKLTVL

SEQ ID NO: 306 - protein, 13 aa, synthetic construct
  REGION 1..13: note = antibody sequence
SGSSSNIGSNTVN

SEQ ID NO: 307 - protein, 7 aa, synthetic construct
  REGION 1..7: note = antibody sequence
YDDLRPS

SEQ ID NO: 308 - protein, 12 aa, synthetic construct
  REGION 1..12: note = antibody sequence
AAWDDSLNVYVV

SEQ ID NO: 309 - DNA, 360 bp, synthetic construct; identical to SEQ ID NO: 289
  misc_feature 1..360: note = antibody sequence
  source 1..360: mol_type = other DNA

SEQ ID NO: 310 - DNA, 15 bp, synthetic construct
  misc_feature 1..15: note = antibody sequence
  source 1..15: mol_type = other DNA
agctacgccatgctg

SEQ ID NO: 311 - DNA, 48 bp, synthetic construct; identical to SEQ ID NO: 151
  misc_feature 1..48: note = antibody sequence
  source 1..48: mol_type = other DNA

SEQ ID NO: 312 - DNA, 36 bp, synthetic construct; identical to SEQ ID NO: 132
  misc_feature 1..36: note = antibody sequence
  source 1..36: mol_type = other DNA

SEQ ID NO: 313 - DNA, 333 bp, synthetic construct
  misc_feature 1..333: note = antibody sequence
  source 1..333: mol_type = other DNA
cagtctgttctgacacagcctcctagcgcctctggcacacctggacagagagtgaccatcagctgtagcggcagcagctccaacatcggcagcaacaccgtgaactggtatcagcagctgcctggcacagcccctaaactgctgatctactacgacgacctgcggcctagcggcgtgccagatagattttctggcagcaagagcggcacctctgccagcctggctatttctggactgcagagcgaggacgaggccgactattattgtgccgcctgggacgacagcctgaacgtgtacgttgtgtttggcggaggcaccaagctgaccgttcta

SEQ ID NO: 314 - DNA, 39 bp, synthetic construct; identical to SEQ ID NO: 54
  misc_feature 1..39: note = antibody sequence
  source 1..39: mol_type = other DNA

SEQ ID NO: 315 - DNA, 21 bp, synthetic construct
  misc_feature 1..21: note = antibody sequence
  source 1..21: mol_type = other DNA
tacgacgacctgcggcctagc

SEQ ID NO: 316 - DNA, 36 bp, synthetic construct
  misc_feature 1..36: note = antibody sequence
  source 1..36: mol_type = other DNA
gccgcctgggacgacagcctgaacgtgtacgttgtg

SEQ ID NO: 317 - protein, 447 aa, synthetic construct; identical to SEQ ID NO: 297
  REGION 1..447: note = antibody sequence

SEQ ID NO: 318 - protein, 217 aa, synthetic construct
  REGION 1..217: note = antibody sequence
QSVLTQPPSASGTPGQRVTISCSGSSSNIGSNTVNWYQQLPGTAPKLLIYYDDLRPSGVPDRFSGSKSGTSASLAISGLQSEDEADYYCAAWDDSLNVYVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 319 - DNA, 1341 bp, synthetic construct; identical to SEQ ID NO: 299
  misc_feature 1..1341: note = antibody sequence
  source 1..1341: mol_type = other DNA

SEQ ID NO: 320 - DNA, 651 bp, synthetic construct
  misc_feature 1..651: note = antibody sequence
  source 1..651: mol_type = other DNA
cagtctgttctgacacagcctcctagcgcctctggcacacctggacagagagtgaccatcagctgtagcggcagcagctccaacatcggcagcaacaccgtgaactggtatcagcagctgcctggcacagcccctaaactgctgatctactacgacgacctgcggcctagcggcgtgccagatagattttctggcagcaagagcggcacctctgccagcctggctatttctggactgcagagcgaggacgaggccgactattattgtgccgcctgggacgacagcctgaacgtgtacgttgtgtttggcggaggcaccaagctgaccgttctaggccagcctaaagccgcccctagcgtgaccctgttccctccaagcagcgaggaactgcaggccaacaaggccaccctcgtgtgcctgatcagcgacttctatcctggcgccgtgaccgtggcctggaaggccgatagctctcctgtgaaggccggcgtggaaaccaccacccctagcaagcagagcaacaacaaatacgccgccagcagctacctgagcctgacccccgagcagtggaagtcccacagatcctacagctgccaagtgacccacgagggcagcaccgtggaaaagacagtggcccctaccgagtgcagc

SEQ ID NO: 321 - protein, 120 aa, synthetic construct
  REGION 1..120: note = antibody sequence
EVQLLESGGGLVQPGGSLRLSCAASGFTFYSYAMSWVRQAPGKGLEWVSAIGYGGDTYYADSVKGRFTISRDNSKNTLYLQMNSLRAEDTAVYYCARRDDYTSRDAFDYWGQGTLVTVSS

SEQ ID NO: 322 - protein, 5 aa, synthetic construct
  REGION 1..5: note = antibody sequence
SYAMS

SEQ ID NO: 323 - protein, 16 aa, synthetic construct
  REGION 1..16: note = antibody sequence
AIGYGGDTYYADSVKG

SEQ ID NO: 324 - protein, 12 aa, synthetic construct
  REGION 1..12: note = antibody sequence
RDDYTSRDAFDY

SEQ ID NO: 325 - protein, 111 aa, synthetic construct; identical to SEQ ID NO: 245
  REGION 1..111: note = antibody sequence

SEQ ID NO: 326 - protein, 13 aa, synthetic construct
  REGION 1..13: note = antibody sequence
SGSSSNIGSNTVN

SEQ ID NO: 327 - protein, 7 aa, synthetic construct
  REGION 1..7: note = antibody sequence
YDDLRPS

SEQ ID NO: 328 - protein, 12 aa, synthetic construct
  REGION 1..12: note = antibody sequence
HAWDDSLNVYPV

SEQ ID NO: 329 - DNA, 360 bp, synthetic construct
  misc_feature 1..360: note = antibody sequence
  source 1..360: mol_type = other DNA
gaagttcagctgctggaatctggcggcggactggttcaacctggcggatctctgagactgagctgtgccgccagcggcttcaccttttacagctacgccatgagctgggtccgacaggcccctggaaaaggccttgaatgggtgtccgccatcggctatggcggcgatacctactacgccgactctgtgaagggcagattcaccatcagccgggacaacagcaagaacaccctgtacctgcagatgaacagcctgagagccgaggacaccgccgtgtactattgcgccagaagggacgactacaccagcagggacgccttcgattattggggccagggcacactggtcaccgtttcttca

SEQ ID NO: 330 - DNA, 15 bp, synthetic construct
  misc_feature 1..15: note = antibody sequence
  source 1..15: mol_type = other DNA
agctacgccatgagc

SEQ ID NO: 331 - DNA, 48 bp, synthetic construct; identical to SEQ ID NO: 171
  misc_feature 1..48: note = antibody sequence
  source 1..48: mol_type = other DNA

SEQ ID NO: 332 - DNA, 36 bp, synthetic construct; identical to SEQ ID NO: 132
  misc_feature 1..36: note = antibody sequence
  source 1..36: mol_type = other DNA

SEQ ID NO: 333 - DNA, 333 bp, synthetic construct; identical to SEQ ID NO: 253
  misc_feature 1..333: note = antibody sequence
  source 1..333: mol_type = other DNA

SEQ ID NO: 334 - DNA, 39 bp, synthetic construct; identical to SEQ ID NO: 54
  misc_feature 1..39: note = antibody sequence
  source 1..39: mol_type = other DNA

SEQ ID NO: 335 - DNA, 21 bp, synthetic construct
  misc_feature 1..21: note = antibody sequence
  source 1..21: mol_type = other DNA
tacgacgacctgcggcctagc

SEQ ID NO: 336 - DNA, 36 bp, synthetic construct; identical to SEQ ID NO: 256
  misc_feature 1..36: note = antibody sequence
  source 1..36: mol_type = other DNA

SEQ ID NO: 337 - protein, 447 aa, synthetic construct
  REGION 1..447: note = antibody sequence
EVQLLESGGGLVQPGGSLRLSCAASGFTFYSYAMSWVRQAPGKGLEWVSAIGYGGDTYYADSVKGRFTISRDNSKNTLYLQMNSLRAEDTAVYYCARRDDYTSRDAFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 338 - protein, 217 aa, synthetic construct; identical to SEQ ID NO: 258
  REGION 1..217: note = antibody sequence

SEQ ID NO: 339 - DNA, 1341 bp, synthetic construct
  misc_feature 1..1341: note = antibody sequence
  source 1..1341: mol_type = other DNA
gaagttcagctgctggaatctggcggcggactggttcaacctggcggatctctgagactgagctgtgccgccagcggcttcaccttttacagctacgccatgagctgggtccgacaggcccctggaaaaggccttgaatgggtgtccgccatcggctatggcggcgatacctactacgccgactctgtgaagggcagattcaccatcagccgggacaacagcaagaacaccctgtacctgcagatgaacagcctgagagccgaggacaccgccgtgtactattgcgccagaagggacgactacaccagcagggacgccttcgattattggggccagggcacactggtcaccgtttcttcagccagcaccaagggccccagcgtgttccctctggccccttgtagcagaagcaccagcgagtctacagccgccctgggctgcctcgtgaaggactactttcccgagcccgtgaccgtgtcctggaactctggcgctctgacaagcggcgtgcacacctttccagccgtgctgcagagcagcggcctgtactctctgagcagcgtcgtgacagtgcccagcagcagcctgggcaccaagacctacacctgtaacgtggaccacaagcccagcaacaccaaggtggacaagcgggtggaatctaagtacggccctccctgccctccttgcccagcccctgaatttctgggcggaccctccgtgttcctgttccccccaaagcccaaggacaccctgatgatcagccggacccccgaagtgacctgcgtggtggtggatgtgtcccaggaagatcccgaggtgcagttcaattggtacgtggacggcgtggaagtgcacaacgccaagaccaagcccagagaggaacagttcaacagcacctaccgggtggtgtccgtgctgacagtgctgcaccaggactggctgaacggcaaagagtacaagtgcaaggtgtccaacaagggcctgcccagctccatcgagaaaaccatcagcaaggccaagggccagccccgcgaaccccaggtgtacacactgcctccaagccaggaagagatgaccaagaaccaggtgtccctgacctgtctcgtgaaaggcttctacccctccgatatcgccgtggaatgggagagcaacggccagcccgagaacaactacaagaccaccccccctgtgctggacagcgacggctcattcttcctgtacagcagactgaccgtggacaagagccggtggcaggaaggcaacgtgttcagctgcagcgtgatgcacgaggccctgcacaaccactacacccagaagtccctgtctctgagcctgggcaag

SEQ ID NO: 340 - DNA, 651 bp, synthetic construct; identical to SEQ ID NO: 260
  misc_feature 1..651: note = antibody sequence
  source 1..651: mol_type = other DNA

SEQ ID NO: 341 - protein, 120 aa, synthetic construct; identical to SEQ ID NO: 321
  REGION 1..120: note = antibody sequence

SEQ ID NO: 342 - protein, 5 aa, synthetic construct
  REGION 1..5: note = antibody sequence
SYAMS

SEQ ID NO: 343 - protein, 16 aa, synthetic construct
  REGION 1..16: note = antibody sequence
AIGYGGDTYYADSVKG

SEQ ID NO: 344 - protein, 12 aa, synthetic construct
  REGION 1..12: note = antibody sequence
RDDYTSRDAFDY

SEQ ID NO: 345 - protein, 111 aa, synthetic construct
  REGION 1..111: note = antibody sequence
QSVLTQPPSASGTPGQRVTISCSGSSSNIGSNTVNWYQQLPGTAPKLLIYYDDLRPSGVPDRFSGSKSGTSASLAISGLQSEDEADYYCAAWDDSLNDIPVFGGGTKLTVL

SEQ ID NO: 346 - protein, 13 aa, synthetic construct
  REGION 1..13: note = antibody sequence
SGSSSNIGSNTVN

SEQ ID NO: 347 - protein, 7 aa, synthetic construct
  REGION 1..7: note = antibody sequence
YDDLRPS

SEQ ID NO: 348 - protein, 12 aa, synthetic construct
  REGION 1..12: note = antibody sequence
AAWDDSLNDIPV

SEQ ID NO: 349 - DNA, 360 bp, synthetic construct; identical to SEQ ID NO: 329
  misc_feature 1..360: note = antibody sequence
  source 1..360: mol_type = other DNA

SEQ ID NO: 350 - DNA, 15 bp, synthetic construct
  misc_feature 1..15: note = antibody sequence
  source 1..15: mol_type = other DNA
agctacgccatgagc

SEQ ID NO: 351 - DNA, 48 bp, synthetic construct; identical to SEQ ID NO: 171
  misc_feature 1..48: note = antibody sequence
  source 1..48: mol_type = other DNA

SEQ ID NO: 352 - DNA, 36 bp, synthetic construct; identical to SEQ ID NO: 132
  misc_feature 1..36: note = antibody sequence
  source 1..36: mol_type = other DNA

SEQ ID NO: 353 - DNA, 333 bp, synthetic construct
  misc_feature 1..333: note = antibody sequence
  source 1..333: mol_type = other DNA
cagtctgttctgacacagcctcctagcgcctctggcacacctggacagagagtgaccatcagctgtagcggcagcagctccaacatcggcagcaacaccgtgaactggtatcagcagctgcctggcacagcccctaaactgctgatctactacgacgacctgcggcctagcggcgtgccagatagattttctggcagcaagagcggcacctctgccagcctggctatttctggactgcagagcgaggacgaggccgactattattgtgccgcctgggacgacagcctgaacgacatccctgtttttggcggaggcaccaagctgaccgttcta

SEQ ID NO: 354 - DNA, 39 bp, synthetic construct; identical to SEQ ID NO: 54
  misc_feature 1..39: note = antibody sequence
  source 1..39: mol_type = other DNA

SEQ ID NO: 355 - DNA, 21 bp, synthetic construct
  misc_feature 1..21: note = antibody sequence
  source 1..21: mol_type = other DNA
tacgacgacctgcggcctagc

SEQ ID NO: 356 - DNA, 36 bp, synthetic construct
  misc_feature 1..36: note = antibody sequence
  source 1..36: mol_type = other DNA
gccgcctgggacgacagcctgaacgacatccctgtt

SEQ ID NO: 357 - protein, 447 aa, synthetic construct; identical to SEQ ID NO: 337
  REGION 1..447: note = antibody sequence

SEQ ID NO: 358 - protein, 217 aa, synthetic construct
  REGION 1..217: note = antibody sequence
QSVLTQPPSASGTPGQRVTISCSGSSSNIGSNTVNWYQQLPGTAPKLLIYYDDLRPSGVPDRFSGSKSGTSASLAISGLQSEDEADYYCAAWDDSLNDIPVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 359 - DNA, 1341 bp, synthetic construct; identical to SEQ ID NO: 339
  misc_feature 1..1341: note = antibody sequence
  source 1..1341: mol_type = other DNA

SEQ ID NO: 360 - DNA, 651 bp, synthetic construct
  misc_feature 1..651: note = antibody sequence
  source 1..651: mol_type = other DNA
cagtctgttctgacacagcctcctagcgcctctggcacacctggacagagagtgaccatcagctgtagcggcagcagctccaacatcggcagcaacaccgtgaactggtatcagcagctgcctggcacagcccctaaactgctgatctactacgacgacctgcggcctagcggcgtgccagatagattttctggcagcaagagcggcacctctgccagcctggctatttctggactgcagagcgaggacgaggccgactattattgtgccgcctgggacgacagcctgaacgacatccctgtttttggcggaggcaccaagctgaccgttctaggccagcctaaagccgcccctagcgtgaccctgttccctccaagcagcgaggaactgcaggccaacaaggccaccctcgtgtgcctgatcagcgacttctatcctggcgccgtgaccgtggcctggaaggccgatagctctcctgtgaaggccggcgtggaaaccaccacccctagcaagcagagcaacaacaaatacgccgccagcagctacctgagcctgacccccgagcagtggaagtcccacagatcctacagctgccaagtgacccacgagggcagcaccgtggaaaagacagtggcccctaccgagtgcagc

SEQ ID NO: 361 - protein, 120 aa, synthetic construct; identical to SEQ ID NO: 321
  REGION 1..120: note = antibody sequence

SEQ ID NO: 362 - protein, 5 aa, synthetic construct
  REGION 1..5: note = antibody sequence
SYAMS

SEQ ID NO: 363 - protein, 16 aa, synthetic construct
  REGION 1..16: note = antibody sequence
AIGYGGDTYYADSVKG

SEQ ID NO: 364 - protein, 12 aa, synthetic construct
  REGION 1..12: note = antibody sequence
RDDYTSRDAFDY

SEQ ID NO: 365 - protein, 111 aa, synthetic construct
  REGION 1..111: note = antibody sequence
QSVLTQPPSASGTPGQRVTISCSGSSSNIGSNTVNWYQQLPGTAPKLLIYYDDLRPSGVPDRFSGSKSGTSASLAISGLQSEDEADYYCAAWDDSLNVIPVFGGGTKLTVL

SEQ ID NO: 366 - protein, 13 aa, synthetic construct
  REGION 1..13: note = antibody sequence
SGSSSNIGSNTVN

SEQ ID NO: 367 - protein, 7 aa, synthetic construct
  REGION 1..7: note = antibody sequence
YDDLRPS

SEQ ID NO: 368 - protein, 12 aa, synthetic construct
  REGION 1..12: note = antibody sequence
AAWDDSLNVIPV

SEQ ID NO: 369 - DNA, 360 bp, synthetic construct; identical to SEQ ID NO: 329
  misc_feature 1..360: note = antibody sequence
  source 1..360: mol_type = other DNA

SEQ ID NO: 370 - DNA, 15 bp, synthetic construct
  misc_feature 1..15: note = antibody sequence
  source 1..15: mol_type = other DNA
agctacgccatgagc

SEQ ID NO: 371 - DNA, 48 bp, synthetic construct; identical to SEQ ID NO: 171
  misc_feature 1..48: note = antibody sequence
  source 1..48: mol_type = other DNA

SEQ ID NO: 372 - DNA, 36 bp, synthetic construct; identical to SEQ ID NO: 132
  misc_feature 1..36: note = antibody sequence
  source 1..36: mol_type = other DNA

SEQ ID NO: 373 - DNA, 333 bp, synthetic construct
  misc_feature 1..333: note = antibody sequence
  source 1..333: mol_type = other DNA
cagtctgttctgacacagcctcctagcgcctctggcacacctggacagagagtgaccatcagctgtagcggcagcagctccaacatcggcagcaacaccgtgaactggtatcagcagctgcctggcacagcccctaaactgctgatctactacgacgacctgcggcctagcggcgtgccagatagattttctggcagcaagagcggcacctctgccagcctggctatttctggactgcagagcgaggacgaggccgactattattgtgccgcctgggacgacagcctgaacgtgatccctgtttttggcggaggcaccaagctgaccgttcta

SEQ ID NO: 374 - DNA, 39 bp, synthetic construct; identical to SEQ ID NO: 54
  misc_feature 1..39: note = antibody sequence
  source 1..39: mol_type = other DNA

SEQ ID NO: 375 - DNA, 21 bp, synthetic construct
  misc_feature 1..21: note = antibody sequence
  source 1..21: mol_type = other DNA
tacgacgacctgcggcctagc

SEQ ID NO: 376 - DNA, 36 bp, synthetic construct
  misc_feature 1..36: note = antibody sequence
  source 1..36: mol_type = other DNA
gccgcctgggacgacagcctgaacgtgatccctgtt

SEQ ID NO: 377 - protein, 447 aa, synthetic construct; identical to SEQ ID NO: 337
  REGION 1..447: note = antibody sequence

SEQ ID NO: 378 - protein, 217 aa, synthetic construct
  REGION 1..217: note = antibody sequence
QSVLTQPPSASGTPGQRVTISCSGSSSNIGSNTVNWYQQLPGTAPKLLIYYDDLRPSGVPDRFSGSKSGTSASLAISGLQSEDEADYYCAAWDDSLNVIPVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 379 - DNA, 1341 bp, synthetic construct; identical to SEQ ID NO: 339
  misc_feature 1..1341: note = antibody sequence
  source 1..1341: mol_type = other DNA

SEQ ID NO: 380 - DNA, 651 bp, synthetic construct
  misc_feature 1..651: note = antibody sequence
  source 1..651: mol_type = other DNA
cagtctgttctgacacagcctcctagcgcctctggcacacctggacagagagtgaccatcagctgtagcggcagcagctccaacatcggcagcaacaccgtgaactggtatcagcagctgcctggcacagcccctaaactgctgatctactacgacgacctgcggcctagcggcgtgccagatagattttctggcagcaagagcggcacctctgccagcctggctatttctggactgcagagcgaggacgaggccgactattattgtgccgcctgggacgacagcctgaacgtgatccctgtttttggcggaggcaccaagctgaccgttctaggccagcctaaagccgcccctagcgtgaccctgttccctccaagcagcgaggaactgcaggccaacaaggccaccctcgtgtgcctgatcagcgacttctatcctggcgccgtgaccgtggcctggaaggccgatagctctcctgtgaaggccggcgtggaaaccaccacccctagcaagcagagcaacaacaaatacgccgccagcagctacctgagcctgacccccgagcagtggaagtcccacagatcctacagctgccaagtgacccacgagggcagcaccgtggaaaagacagtggcccctaccgagtgcagc

SEQ ID NO: 381 - protein, 123 aa, synthetic construct
  REGION 1..123: note = antibody sequence
EVQLLESGGGLVQPGGSLRLSCAASGFTFDSYEMNWVRQAPGKGLEWVSGISWNSGWIDYADSVKGRFTISRDNSKNTLYLQMNSLRAEDTAVYYCARSGYSSSWFDPDFDYWGQGTLVTVSS

SEQ ID NO: 382 - protein, 5 aa, synthetic construct
  REGION 1..5: note = antibody sequence
SYEMN

SEQ ID NO: 383 - protein, 17 aa, synthetic construct
  REGION 1..17: note = antibody sequence
GISWNSGWIDYADSVKG

SEQ ID NO: 384 - protein, 14 aa, synthetic construct
  REGION 1..14: note = antibody sequence
SGYSSSWFDPDFDY

SEQ ID NO: 385 - protein, 111 aa, synthetic construct
  REGION 1..111: note = antibody sequence
QSVLTQPPSVSGAPGQRVTISCTGSSSDIGAGYDVHWYQQLPGTAPKLLIYGNSNRPSGVPDRFSGSKSGTSASLAITGLQAEDEADYYCSSYAGPNPYVVFGGGTKLTVL

SEQ ID NO: 386 - protein, 14 aa, synthetic construct
  REGION 1..14: note = antibody sequence
TGSSSDIGAGYDVH

SEQ ID NO: 387 - protein, 7 aa, synthetic construct
  REGION 1..7: note = antibody sequence
GNSNRPS

SEQ ID NO: 388 - protein, 11 aa, synthetic construct
  REGION 1..11: note = antibody sequence
SSYAGPNPYVV

SEQ ID NO: 389 - DNA, 369 bp, synthetic construct
  misc_feature 1..369: note = antibody sequence
  source 1..369: mol_type = other DNA
gaagttcagctgctggaatctggcggcggactggttcaacctggcggatctctgagactgagctgtgccgccagcggcttcaccttcgatagctacgagatgaactgggtccgacaggcccctggcaaaggccttgaatgggtgtccggcatcagctggaatagcggctggatcgactacgccgacagcgtgaagggcagattcaccatcagccgggacaacagcaagaacaccctgtacctgcagatgaacagcctgagagccgaggacaccgccgtgtactactgtgccagaagcggctacagcagctcttggtttgaccccgacttcgactattggggccagggcacactggtcacagtctcttca

SEQ ID NO: 390 - DNA, 15 bp, synthetic construct
  misc_feature 1..15: note = antibody sequence
  source 1..15: mol_type = other DNA
agctacgagatgaac

SEQ ID NO: 391 - DNA, 51 bp, synthetic construct
  misc_feature 1..51: note = antibody sequence
  source 1..51: mol_type = other DNA
ggcatcagctggaatagcggctggatcgactacgccgacagcgtgaagggc

SEQ ID NO: 392 - DNA, 42 bp, synthetic construct; identical to SEQ ID NO: 72
  misc_feature 1..42: note = antibody sequence
  source 1..42: mol_type = other DNA

SEQ ID NO: 393 - DNA, 333 bp, synthetic construct
  misc_feature 1..333: note = antibody sequence
  source 1..333: mol_type = other DNA
cagtctgttctgacacagcctccatctgtgtctggcgcccctggacagagagtgaccatcagctgtacaggcagcagctccgatattggcgccggatacgacgtgcactggtatcagcaactgcctggcacagcccctaagctgctgatctacggcaacagcaacagacctagcggcgtgcccgatagattcagcggctctaagtctggcacaagcgccagcctggccattactggactgcaggccgaagatgaggccgactactactgcagcagctacgctggccccaatccttacgtggtgtttggcggcggaacaaagctgaccgttcta

SEQ ID NO: 394 - DNA, 42 bp, synthetic construct
  misc_feature 1..42: note = antibody sequence
  source 1..42: mol_type = other DNA
acaggcagcagctccgatattggcgccggatacgacgtgcac

SEQ ID NO: 395 - DNA, 21 bp, synthetic construct
  misc_feature 1..21: note = antibody sequence
  source 1..21: mol_type = other DNA
ggcaacagcaacagacctagc

SEQ ID NO: 396 - DNA, 33 bp, synthetic construct
  misc_feature 1..33: note = antibody sequence
  source 1..33: mol_type = other DNA
agcagctacgctggccccaatccttacgtggtg

SEQ ID NO: 397 - protein, 453 aa, synthetic construct
  REGION 1..453: note = antibody sequence
EVQLLESGGGLVQPGGSLRLSCAASGFTFDSYEMNWVRQAPGKGLEWVSGISWNSGWIDYADSVKGRFTISRDNSKNTLYLQMNSLRAEDTAVYYCARSGYSSSWFDPDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 398 - protein, 217 aa, synthetic construct
  REGION 1..217: note = antibody sequence
QSVLTQPPSVSGAPGQRVTISCTGSSSDIGAGYDVHWYQQLPGTAPKLLIYGNSNRPSGVPDRFSGSKSGTSASLAITGLQAEDEADYYCSSYAGPNPYVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 399 - DNA, 1359 bp, synthetic construct
  misc_feature 1..1359: note = antibody sequence
  source 1..1359: mol_type = other DNA
gaagttcagctgctggaatctggcggcggactggttcaacctggcggatctctgagactgagctgtgccgccagcggcttcaccttcgatagctacgagatgaactgggtccgacaggcccctggcaaaggccttgaatgggtgtccggcatcagctggaatagcggctggatcgactacgccgacagcgtgaagggcagattcaccatcagccgggacaacagcaagaacaccctgtacctgcagatgaacagcctgagagccgaggacaccgccgtgtactactgtgccagaagcggctacagcagctcttggtttgaccccgacttcgactattggggccagggcacactggtcacagtctcttcagccagcaccaagggccccagcgtgttccctctggcccctagcagcaagagcacatctggcggaacagccgccctgggctgcctcgtgaaggactactttcccgagcccgtgaccgtgtcctggaactctggcgctctgacaagcggcgtgcacacctttccagccgtgctgcagagcagcggcctgtactctctgagcagcgtcgtgacagtgcccagcagctctctgggcacccagacctacatctgcaacgtgaaccacaagcccagcaacaccaaggtggacaagaaggtggaacccaagagctgcgacaagacccacacctgtcccccttgtcctgcccccgaactgctgggaggcccttccgtgttcctgttccccccaaagcccaaggacaccctgatgatcagccggacccccgaagtgacctgcgtggtggtggatgtgtcccacgaggaccctgaagtgaagttcaattggtacgtggacggcgtggaagtgcacaacgccaagaccaagcctagagaggaacagtacaacagcacctaccgggtggtgtccgtgctgacagtgctgcaccaggactggctgaacggcaaagagtacaagtgcaaggtgtccaacaaggccctgcctgcccccatcgagaaaaccatcagcaaggccaagggccagccccgcgaaccccaggtgtacacactgcccccaagcagggacgagctgaccaagaaccaggtgtccctgacctgtctcgtgaaaggcttctacccctccgatatcgccgtggaatgggagagcaacggccagcccgagaacaactacaagaccaccccccctgtgctggacagcgacggctcattcttcctgtacagcaagctgaccgtggacaagtcccggtggcagcagggcaacgtgttcagctgcagcgtgatgcacgaggccctgcacaaccactacacccagaagtccctgagcctgagccctggcaag

SEQ ID NO: 400 - DNA, 651 bp, synthetic construct
  misc_feature 1..651: note = antibody sequence
  source 1..651: mol_type = other DNA
cagtctgttctgacacagcctccatctgtgtctggcgcccctggacagagagtgaccatcagctgtacaggcagcagctccgatattggcgccggatacgacgtgcactggtatcagcaactgcctggcacagcccctaagctgctgatctacggcaacagcaacagacctagcggcgtgcccgatagattcagcggctctaagtctggcacaagcgccagcctggccattactggactgcaggccgaagatgaggccgactactactgcagcagctacgctggccccaatccttacgtggtgtttggcggcggaacaaagctgaccgttctaggccagcctaaagccgcccctagcgtgaccctgttccctccaagcagcgaggaactgcaggccaacaaggccaccctcgtgtgcctgatcagcgacttctatcctggcgccgtgaccgtggcctggaaggccgatagctctcctgtgaaggccggcgtggaaaccaccacccctagcaagcagagcaacaacaaatacgccgccagcagctacctgagcctgacccccgagcagtggaagtcccacagatcctacagctgccaagtgacccacgagggcagcaccgtggaaaagacagtggcccctaccgagtgcagc

SEQ ID NO: 401 - protein, 123 aa, synthetic construct; identical to SEQ ID NO: 381
  REGION 1..123: note = antibody sequence

SEQ ID NO: 402 - protein, 5 aa, synthetic construct
  REGION 1..5: note = antibody sequence
SYEMN

SEQ ID NO: 403 - protein, 17 aa, synthetic construct
  REGION 1..17: note = antibody sequence
GISWNSGWIDYADSVKG

SEQ ID NO: 404 - protein, 14 aa, synthetic construct
  REGION 1..14: note = antibody sequence
SGYSSSWFDPDFDY

SEQ ID NO: 405 - protein, 111 aa, synthetic construct
  REGION 1..111: note = antibody sequence
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYAGINPYVVFGGGTKLTVL

SEQ ID NO: 406 - protein, 14 aa, synthetic construct
  REGION 1..14: note = antibody sequence
TGSSSNIGAGYDVH

SEQ ID NO: 407 - protein, 7 aa, synthetic construct
  REGION 1..7: note = antibody sequence
GNSNRPS

SEQ ID NO: 408 - protein, 11 aa, synthetic construct
  REGION 1..11: note = antibody sequence
QSYAGINPYVV

SEQ ID NO: 409 - DNA, 369 bp, synthetic construct; identical to SEQ ID NO: 389
  misc_feature 1..369: note = antibody sequence
  source 1..369: mol_type = other DNA

SEQ ID NO: 410 - DNA, 15 bp, synthetic construct
  misc_feature 1..15: note = antibody sequence
  source 1..15: mol_type = other DNA
agctacgagatgaac

SEQ ID NO: 411 - DNA, 51 bp, synthetic construct; identical to SEQ ID NO: 391
  misc_feature 1..51: note = antibody sequence
  source 1..51: mol_type = other DNA

SEQ ID NO: 412 - DNA, 42 bp, synthetic construct; identical to SEQ ID NO: 72
  misc_feature 1..42: note = antibody sequence
  source 1..42: mol_type = other DNA

SEQ ID NO: 413 - DNA, 333 bp, synthetic construct
  misc_feature 1..333: note = antibody sequence
  source 1..333: mol_type = other DNA
cagtctgttctgacacagcctccatctgtgtctggcgcccctggacagagagtgaccatcagctgtacaggcagcagctccaatatcggagccggctatgacgtgcactggtatcagcagctgcctggcacagcccctaaactgctgatctacggcaacagcaacagacccagcggcgtgcccgatagattttccggctctaagagcggcacaagcgccagcctggctattactggactgcaggccgaggacgaggccgactactactgtcagagctacgccggcatcaacccctacgtggtgtttggcggaggcaccaagctgacagttcta

SEQ ID NO: 414 - DNA, 42 bp, synthetic construct; identical to SEQ ID NO: 114
  misc_feature 1..42: note = antibody sequence
  source 1..42: mol_type = other DNA

SEQ ID NO: 415 - DNA, 21 bp, synthetic construct
  misc_feature 1..21: note = antibody sequence
  source 1..21: mol_type = other DNA
ggcaacagcaacagacccagc

SEQ ID NO: 416 - DNA, 33 bp, synthetic construct
  misc_feature 1..33: note = antibody sequence
  source 1..33: mol_type = other DNA
cagagctacgccggcatcaacccctacgtggtg

SEQ ID NO: 417 - protein, 453 aa, synthetic construct; identical to SEQ ID NO: 397
  REGION 1..453: note = antibody sequence

SEQ ID NO: 418 - protein, 217 aa, synthetic construct
  REGION 1..217: note = antibody sequence
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYAGINPYVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 419 - DNA, 1359 bp, synthetic construct; identical to SEQ ID NO: 399
  misc_feature 1..1359: note = antibody sequence
  source 1..1359: mol_type = other DNA

SEQ ID NO: 420 - DNA, 651 bp, synthetic construct
  misc_feature 1..651: note = antibody sequence
  source 1..651: mol_type = other DNA
cagtctgttctgacacagcctccatctgtgtctggcgcccctggacagagagtgaccatcagctgtacaggcagcagctccaatatcggagccggctatgacgtgcactggtatcagcagctgcctggcacagcccctaaactgctgatctacggcaacagcaacagacccagcggcgtgcccgatagattttccggctctaagagcggcacaagcgccagcctggctattactggactgcaggccgaggacgaggccgactactactgtcagagctacgccggcatcaacccctacgtggtgtttggcggaggcaccaagctgacagttctaggccagcctaaagccgcccctagcgtgaccctgttccctccaagcagcgaggaactgcaggccaacaaggccaccctcgtgtgcctgatcagcgacttctatcctggcgccgtgaccgtggcctggaaggccgatagctctcctgtgaaggccggcgtggaaaccaccacccctagcaagcagagcaacaacaaatacgccgccagcagctacctgagcctgacccccgagcagtggaagtcccacagatcctacagctgccaagtgacccacgagggcagcaccgtggaaaagacagtggcccctaccgagtgcagc

SEQ ID NO: 421 - protein, 123 aa, synthetic construct
  REGION 1..123: note = antibody sequence
EVQLLESGGGLVQPGGSLRLSCAASGFDFSSYEMNWVRQAPGKGLEWVSGISWNSGWIGYADSVKGRFTISRDNSKNTLYLQMNSLRAEDTAVYYCARSGYSSSWFDPDFDYWGQGTLVTVSS

SEQ ID NO: 422 - protein, 5 aa, synthetic construct
  REGION 1..5: note = antibody sequence
SYEMN

SEQ ID NO: 423 - protein, 17 aa, synthetic construct
  REGION 1..17: note = antibody sequence
GISWNSGWIGYADSVKG

SEQ ID NO: 424 - protein, 14 aa, synthetic construct
  REGION 1..14: note = antibody sequence
SGYSSSWFDPDFDY

SEQ ID NO: 425 - protein, 111 aa, synthetic construct
  REGION 1..111: note = antibody sequence
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYAGPNPYVVFGGGTKLTVL

SEQ ID NO: 426 - protein, 14 aa, synthetic construct
  REGION 1..14: note = antibody sequence
TGSSSNIGAGYDVH

SEQ ID NO: 427 - protein, 7 aa, synthetic construct
  REGION 1..7: note = antibody sequence
GNSNRPS

SEQ ID NO: 428 - protein, 11 aa, synthetic construct
  REGION 1..11: note = antibody sequence
QSYAGPNPYVV

SEQ ID NO: 429 - DNA, 369 bp, synthetic construct
  misc_feature 1..369: note = antibody sequence
  source 1..369: mol_type = other DNA
gaagttcagctgctggaatctggcggcggactggttcaacctggcggatctctgagactgagctgtgccgccagcggcttcgatttcagcagctacgagatgaactgggtccgacaggcccctggcaaaggccttgaatgggtgtccggcatcagctggaatagcggctggatcggctacgccgatagcgtgaagggcagattcaccatcagccgggacaacagcaagaacaccctgtacctgcagatgaacagcctgagagccgaggacaccgccgtgtactactgtgccagaagcggctacagcagctcttggtttgaccccgacttcgactattggggccagggcacactggtcacagtctcttca

SEQ ID NO: 430 - DNA, 15 bp, synthetic construct
  misc_feature 1..15: note = antibody sequence
  source 1..15: mol_type = other DNA
agctacgagatgaac

SEQ ID NO: 431 - DNA, 51 bp, synthetic construct
  misc_feature 1..51: note = antibody sequence
  source 1..51: mol_type = other DNA
ggcatcagctggaatagcggctggatcggctacgccgatagcgtgaagggc

SEQ ID NO: 432 - DNA, 42 bp, synthetic construct; identical to SEQ ID NO: 72
  misc_feature 1..42: note = antibody sequence
  source 1..42: mol_type = other DNA

SEQ ID NO: 433 - DNA, 333 bp, synthetic construct
  misc_feature 1..333: note = antibody sequence
  source 1..333: mol_type = other DNA
cagtctgttctgacacagcctccatctgtgtctggcgcccctggacagagagtgaccatcagctgtacaggcagcagctccaatatcggagccggctatgacgtgcactggtatcagcagctgcctggcacagcccctaaactgctgatctacggcaacagcaacagacccagcggcgtgcccgatagattttccggctctaagagcggcacaagcgccagcctggctattactggactgcaggccgaggacgaggccgactactactgtcagtcttacgctggccccaatccttacgtggtgtttggcggcggaacaaagctgaccgttcta

SEQ ID NO: 434 - DNA, 42 bp, synthetic construct; identical to SEQ ID NO: 114
  misc_feature 1..42: note = antibody sequence
  source 1..42: mol_type = other DNA

SEQ ID NO: 435 - DNA, 21 bp, synthetic construct
  misc_feature 1..21: note = antibody sequence
  source 1..21: mol_type = other DNA
ggcaacagcaacagacccagc

SEQ ID NO: 436 - DNA, 33 bp, synthetic construct
  misc_feature 1..33: note = antibody sequence
  source 1..33: mol_type = other DNA
cagtcttacgctggccccaatccttacgtggtg

SEQ ID NO: 437 - protein, 453 aa, synthetic construct
  REGION 1..453: note = antibody sequence
EVQLLESGGGLVQPGGSLRLSCAASGFDFSSYEMNWVRQAPGKGLEWVSGISWNSGWIGYADSVKGRFTISRDNSKNTLYLQMNSLRAEDTAVYYCARSGYSSSWFDPDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 438 - protein, 217 aa, synthetic construct
  REGION 1..217: note = antibody sequence
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYAGPNPYVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 439 - DNA, 1359 bp, synthetic construct
  misc_feature 1..1359: note = antibody sequence
  source 1..1359: mol_type = other DNA
gaagttcagctgctggaatctggcggcggactggttcaacctggcggatctctgagactgagctgtgccgccagcggcttcgatttcagcagctacgagatgaactgggtccgacaggcccctggcaaaggccttgaatgggtgtccggcatcagctggaatagcggctggatcggctacgccgatagcgtgaagggcagattcaccatcagccgggacaacagcaagaacaccctgtacctgcagatgaacagcctgagagccgaggacaccgccgtgtactactgtgccagaagcggctacagcagctcttggtttgaccccgacttcgactattggggccagggcacactggtcacagtctcttcagccagcaccaagggccccagcgtgttccctctggcccctagcagcaagagcacatctggcggaacagccgccctgggctgcctcgtgaaggactactttcccgagcccgtgaccgtgtcctggaactctggcgctctgacaagcggcgtgcacacctttccagccgtgctgcagagcagcggcctgtactctctgagcagcgtcgtgacagtgcccagcagctctctgggcacccagacctacatctgcaacgtgaaccacaagcccagcaacaccaaggtggacaagaaggtggaacccaagagctgcgacaagacccacacctgtcccccttgtcctgcccccgaactgctgggaggcccttccgtgttcctgttccccccaaagcccaaggacaccctgatgatcagccggacccccgaagtgacctgcgtggtggtggatgtgtcccacgaggaccctgaagtgaagttcaattggtacgtggacggcgtggaagtgcacaacgccaagaccaagcctagagaggaacagtacaacagcacctaccgggtggtgtccgtgctgacagtgctgcaccaggactggctgaacggcaaagagtacaagtgcaaggtgtccaacaaggccctgcctgcccccatcgagaaaaccatcagcaaggccaagggccagccccgcgaaccccaggtgtacacactgcccccaagcagggacgagctgaccaagaaccaggtgtccctgacctgtctcgtgaaaggcttctacccctccgatatcgccgtggaatgggagagcaacggccagcccgagaacaactacaagaccaccccccctgtgctggacagcgacggctcattcttcctgtacagcaagctgaccgtggacaagtcccggtggcagcagggcaacgtgttcagctgcagcgtgatgcacgaggccctgcacaaccactacacccagaagtccctgagcctgagccctggcaag

SEQ ID NO: 440 - DNA, 651 bp, synthetic construct
  misc_feature 1..651: note = antibody sequence
  source 1..651: mol_type = other DNA
cagtctgttctgacacagcctccatctgtgtctggcgcccctggacagagagtgaccatcagctgtacaggcagcagctccaatatcggagccggctatgacgtgcactggtatcagcagctgcctggcacagcccctaaactgctgatctacggcaacagcaacagacccagcggcgtgcccgatagattttccggctctaagagcggcacaagcgccagcctggctattactggactgcaggccgaggacgaggccgactactactgtcagtcttacgctggccccaatccttacgtggtgtttggcggcggaacaaagctgaccgttctaggccagcctaaagccgcccctagcgtgaccctgttccctccaagcagcgaggaactgcaggccaacaaggccaccctcgtgtgcctgatcagcgacttctatcctggcgccgtgaccgtggcctggaaggccgatagctctcctgtgaaggccggcgtggaaaccaccacccctagcaagcagagcaacaacaaatacgccgccagcagctacctgagcctgacccccgagcagtggaagtcccacagatcctacagctgccaagtgacccacgagggcagcaccgtggaaaagacagtggcccctaccgagtgcagc

SEQ ID NO: 441 - protein, 123 aa, synthetic construct
  REGION 1..123: note = antibody sequence
EVQLLESGGGLVQPGGSLRLSCAASGFDFSSYEMNWVRQAPGKGLEWVSGISWNSGWIDYADSVKGRFTISRDNSKNTLYLQMNSLRAEDTAVYYCARSGYSSSWFDPDFDYWGQGTLVTVSS

SEQ ID NO: 442 - protein, 5 aa, synthetic construct
  REGION 1..5: note = antibody sequence
SYEMN

SEQ ID NO: 443 - protein, 17 aa, synthetic construct
  REGION 1..17: note = antibody sequence
GISWNSGWIDYADSVKG

SEQ ID NO: 444 - protein, 14 aa, synthetic construct
  REGION 1..14: note = antibody sequence
SGYSSSWFDPDFDY

SEQ ID NO: 445 - protein, 111 aa, synthetic construct; identical to SEQ ID NO: 425
  REGION 1..111: note = antibody sequence

SEQ ID NO: 446 - protein, 14 aa, synthetic construct
  REGION 1..14: note = antibody sequence
TGSSSNIGAGYDVH

SEQ ID NO: 447 - protein, 7 aa, synthetic construct
  REGION 1..7: note = antibody sequence
GNSNRPS

SEQ ID NO: 448 - protein, 11 aa, synthetic construct
  REGION 1..11: note = antibody sequence
QSYAGPNPYVV

SEQ ID NO: 449 - DNA, 369 bp, synthetic construct
  misc_feature 1..369: note = antibody sequence
  source 1..369: mol_type = other DNA
gaagttcagctgctggaatctggcggcggactggttcaacctggcggatctctgagactgagctgtgccgccagcggcttcgatttcagcagctacgagatgaactgggtccgacaggcccctggcaaaggccttgaatgggtgtccggcatcagctggaatagcggctggatcgactacgccgacagcgtgaagggcagattcaccatcagccgggacaacagcaagaacaccctgtacctgcagatgaacagcctgagagccgaggacaccgccgtgtactactgtgccagaagcggctacagcagctcttggtttgaccccgacttcgactattggggccagggcacactggtcacagtctcttca

SEQ ID NO: 450 - DNA, 15 bp, synthetic construct
  misc_feature 1..15: note = antibody sequence
  source 1..15: mol_type = other DNA
agctacgagatgaac

SEQ ID NO: 451 - DNA, 51 bp, synthetic construct; identical to SEQ ID NO: 391
  misc_feature 1..51: note = antibody sequence
  source 1..51: mol_type = other DNA

SEQ ID NO: 452 - DNA, 42 bp, synthetic construct; identical to SEQ ID NO: 72
  misc_feature 1..42: note = antibody sequence
  source 1..42: mol_type = other DNA

SEQ ID NO: 453 - DNA, 333 bp, synthetic construct; identical to SEQ ID NO: 433
  misc_feature 1..333: note = antibody sequence
  source 1..333: mol_type = other DNA

SEQ ID NO: 454 - DNA, 42 bp, synthetic construct; identical to SEQ ID NO: 114
  misc_feature 1..42: note = antibody sequence
  source 1..42: mol_type = other DNA

SEQ ID NO: 455 - DNA, 21 bp, synthetic construct
  misc_feature 1..21: note = antibody sequence
  source 1..21: mol_type = other DNA
ggcaacagcaacagacccagc

SEQ ID NO: 456 - DNA, 33 bp, synthetic construct; identical to SEQ ID NO: 436
  misc_feature 1..33: note = antibody sequence
  source 1..33: mol_type = other DNA

SEQ ID NO: 457 - protein, 453 aa, synthetic construct
  REGION 1..453: note = antibody sequence
EVQLLESGGGLVQPGGSLRLSCAASGFDFSSYEMNWVRQAPGKGLEWVSGISWNSGWIDYADSVKGRFTISRDNSKNTLYLQMNSLRAEDTAVYYCARSGYSSSWFDPDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 458 - protein, 217 aa, synthetic construct; identical to SEQ ID NO: 438
  REGION 1..217: note = antibody sequence

SEQ ID NO: 459 - DNA, 1359 bp, synthetic construct
  misc_feature 1..1359: note = antibody sequence
  source 1..1359: mol_type = other DNA
gaagttcagctgctggaatctggcggcggactggttcaacctggcggatctctgagactgagctgtgccgccagcggcttcgatttcagcagctacgagatgaactgggtccgacaggcccctggcaaaggccttgaatgggtgtccggcatcagctggaatagcggctggatcgactacgccgacagcgtgaagggcagattcaccatcagccgggacaacagcaagaacaccctgtacctgcagatgaacagcctgagagccgaggacaccgccgtgtactactgtgccagaagcggctacagcagctcttggtttgaccccgacttcgactattggggccagggcacactggtcacagtctcttcagccagcaccaagggccccagcgtgttccctctggcccctagcagcaagagcacatctggcggaacagccgccctgggctgcctcgtgaaggactactttcccgagcccgtgaccgtgtcctggaactctggcgctctgacaagcggcgtgcacacctttccagccgtgctgcagagcagcggcctgtactctctgagcagcgtcgtgacagtgcccagcagctctctgggcacccagacctacatctgcaacgtgaaccacaagcccagcaacaccaaggtggacaagaaggtggaacccaagagctgcgacaagacccacacctgtcccccttgtcctgcccccgaactgctgggaggcccttccgtgttcctgttccccccaaagcccaaggacaccctgatgatcagccggacccccgaagtgacctgcgtggtggtggatgtgtcccacgaggaccctgaagtgaagttcaattggtacgtggacggcgtggaagtgcacaacgccaagaccaagcctagagaggaacagtacaacagcacctaccgggtggtgtccgtgctgacagtgctgcaccaggactggctgaacggcaaagagtacaagtgcaaggtgtccaacaaggccctgcctgcccccatcgagaaaaccatcagcaaggccaagggccagccccgcgaaccccaggtgtacacactgcccccaagcagggacgagctgaccaagaaccaggtgtccctgacctgtctcgtgaaaggcttctacccctccgatatcgccgtggaatgggagagcaacggccagcccgagaacaactacaagaccaccccccctgtgctggacagcgacggctcattcttcctgtacagcaagctgaccgtggacaagtcccggtggcagcagggcaacgtgttcagctgcagcgtgatgcacgaggccctgcacaaccactacacccagaagtccctgagcctgagccctggcaag

SEQ ID NO: 460 - DNA, 651 bp, synthetic construct; identical to SEQ ID NO: 440
  misc_feature 1..651: note = antibody sequence
  source 1..651: mol_type = other DNA

SEQ ID NO: 461 - protein, 123 aa, synthetic construct; identical to SEQ ID NO: 381
  REGION 1..123: note = antibody sequence

SEQ ID NO: 462 - protein, 5 aa, synthetic construct
  REGION 1..5: note = antibody sequence
SYEMN

SEQ ID NO: 463 - protein, 17 aa, synthetic construct
  REGION 1..17: note = antibody sequence
GISWNSGWIDYADSVKG

SEQ ID NO: 464 - protein, 14 aa, synthetic construct
  REGION 1..14: note = antibody sequence
SGYSSSWFDPDFDY

SEQ ID NO: 465 - protein, 111 aa, synthetic construct; identical to SEQ ID NO: 425
  REGION 1..111: note = antibody sequence

SEQ ID NO: 466 - protein, 14 aa, synthetic construct
  REGION 1..14: note = antibody sequence
TGSSSNIGAGYDVH

SEQ ID NO: 467 - protein, 7 aa, synthetic construct
  REGION 1..7: note = antibody sequence
GNSNRPS

SEQ ID NO: 468 - protein, 11 aa, synthetic construct
  REGION 1..11: note = antibody sequence
QSYAGPNPYVV

SEQ ID NO: 469 - DNA, 369 bp, synthetic construct; identical to SEQ ID NO: 389
  misc_feature 1..369: note = antibody sequence
  source 1..369: mol_type = other DNA

SEQ ID NO: 470 - DNA, 15 bp, synthetic construct
  misc_feature 1..15: note = antibody sequence
  source 1..15: mol_type = other DNA
agctacgagatgaac

SEQ ID NO: 471 - DNA, 51 bp, synthetic construct; identical to SEQ ID NO: 391
  misc_feature 1..51: note = antibody sequence
  source 1..51: mol_type = other DNA

SEQ ID NO: 472 - DNA, 42 bp, synthetic construct; identical to SEQ ID NO: 72
  misc_feature 1..42: note = antibody sequence
  source 1..42: mol_type = other DNA

SEQ ID NO: 473 - DNA, 333 bp, synthetic construct; identical to SEQ ID NO: 433
  misc_feature 1..333: note = antibody sequence
  source 1..333: mol_type = other DNA

SEQ ID NO: 474 - DNA, 42 bp, synthetic construct; identical to SEQ ID NO: 114
  misc_feature 1..42: note = antibody sequence
  source 1..42: mol_type = other DNA

SEQ ID NO: 475 - DNA, 21 bp, synthetic construct
  misc_feature 1..21: note = antibody sequence
  source 1..21: mol_type = other DNA
ggcaacagcaacagacccagc

SEQ ID NO: 476 - DNA, 33 bp, synthetic construct; identical to SEQ ID NO: 436
  misc_feature 1..33: note = antibody sequence
  source 1..33: mol_type = other DNA

SEQ ID NO: 477 - protein, 453 aa, synthetic construct; identical to SEQ ID NO: 397
  REGION 1..453: note = antibody sequence

SEQ ID NO: 478 - protein, 217 aa, synthetic construct; identical to SEQ ID NO: 438
  REGION 1..217: note = antibody sequence

SEQ ID NO: 479 - DNA, 1359 bp, synthetic construct; identical to SEQ ID NO: 399
  misc_feature 1..1359: note = antibody sequence
  source 1..1359: mol_type = other DNA

SEQ ID NO: 480 - DNA, 651 bp, synthetic construct; identical to SEQ ID NO: 440
  misc_feature 1..651: note = antibody sequence
  source 1..651: mol_type = other DNA

SEQ ID NO: 481 - protein, 123 aa, synthetic construct
  REGION 1..123: note = antibody sequence
EVQLLESGGGLVQPGGSLRLSCAASGFDFDSYEMNWVRQAPGKGLEWVSGISWNSGWIDYADSVKGRFTISRDNSKNTLYLQMNSLRAEDTAVYYCARSGYSSSWFDPDFDYWGQGTLVTVSS

SEQ ID NO: 482 - protein, 5 aa, synthetic construct
  REGION 1..5: note = antibody sequence
SYEMN

SEQ ID NO: 483 - protein, 17 aa, synthetic construct
  REGION 1..17: note = antibody sequence
GISWNSGWIDYADSVKG

SEQ ID NO: 484 - protein, 14 aa, synthetic construct
  REGION 1..14: note = antibody sequence
SGYSSSWFDPDFDY

SEQ ID NO: 485 - protein, 111 aa, synthetic construct; identical to SEQ ID NO: 425
  REGION 1..111: note = antibody sequence

SEQ ID NO: 486 - protein, 14 aa, synthetic construct
  REGION 1..14: note = antibody sequence
TGSSSNIGAGYDVH

SEQ ID NO: 487 - protein, 7 aa, synthetic construct
  REGION 1..7: note = antibody sequence
GNSNRPS

SEQ ID NO: 488 - protein, 11 aa, synthetic construct
  REGION 1..11: note = antibody sequence
QSYAGPNPYVV

SEQ ID NO: 489 - DNA, 369 bp, synthetic construct
  misc_feature 1..369: note = antibody sequence
  source 1..369: mol_type = other DNA
gaagttcagctgctggaatctggcggcggactggttcaacctggcggatctctgagactgagctgtgccgccagcggcttcgacttcgatagctacgagatgaactgggtccgacaggcccctggcaaaggccttgaatgggtgtccggcatcagctggaatagcggctggatcgactacgccgacagcgtgaagggcagattcaccatcagccgggacaacagcaagaacaccctgtacctgcagatgaacagcctgagagccgaggacaccgccgtgtactactgtgccagaagcggctacagcagctcttggtttgaccccgacttcgactattggggccagggcacactggtcacagtctcttca

SEQ ID NO: 490 - DNA, 15 bp, synthetic construct
  misc_feature 1..15: note = antibody sequence
  source 1..15: mol_type = other DNA
agctacgagatgaac

SEQ ID NO: 491 - DNA, 51 bp, synthetic construct; identical to SEQ ID NO: 391
  misc_feature 1..51: note = antibody sequence
  source 1..51: mol_type = other DNA

SEQ ID NO: 492 - DNA, 42 bp, synthetic construct; identical to SEQ ID NO: 72
  misc_feature 1..42: note = antibody sequence
  source 1..42: mol_type = other DNA

SEQ ID NO: 493 - DNA, 333 bp, synthetic construct; identical to SEQ ID NO: 433
  misc_feature 1..333: note = antibody sequence
  source 1..333: mol_type = other DNA

SEQ ID NO: 494 - DNA, 42 bp, synthetic construct; identical to SEQ ID NO: 114
  misc_feature 1..42: note = antibody sequence
  source 1..42: mol_type = other DNA

SEQ ID NO: 495 - DNA, 21 bp, synthetic construct
  misc_feature 1..21: note = antibody sequence
  source 1..21: mol_type = other DNA
ggcaacagcaacagacccagc

SEQ ID NO: 496 - DNA, 33 bp, synthetic construct; identical to SEQ ID NO: 436
  misc_feature 1..33: note = antibody sequence
  source 1..33: mol_type = other DNA

SEQ ID NO: 497 - protein, 453 aa, synthetic construct
  REGION 1..453: note = antibody sequence
EVQLLESGGGLVQPGGSLRLSCAASGFDFDSYEMNWVRQAPGKGLEWVSGISWNSGWIDYADSVKGRFTISRDNSKNTLYLQMNSLRAEDTAVYYCARSGYSSSWFDPDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 498 - protein, 217 aa, synthetic construct; identical to SEQ ID NO: 438
  REGION 1..217: note = antibody sequence

SEQ ID NO: 499 - DNA, 1359 bp, synthetic construct
  misc_feature 1..1359: note = antibody sequence
  source 1..1359: mol_type = other DNA
gaagttcagctgctggaatctggcggcggactggttcaacctggcggatctctgagactgagctgtgccgccagcggcttcgacttcgatagctacgagatgaactgggtccgacaggcccctggcaaaggccttgaatgggtgtccggcatcagctggaatagcggctggatcgactacgccgacagcgtgaagggcagattcaccatcagccgggacaacagcaagaacaccctgtacctgcagatgaacagcctgagagccgaggacaccgccgtgtactactgtgccagaagcggctacagcagctcttggtttgaccccgacttcgactattggggccagggcacactggtcacagtctcttcagccagcaccaagggccccagcgtgttccctctggcccctagcagcaagagcacatctggcggaacagccgccctgggctgcctcgtgaaggactactttcccgagcccgtgaccgtgtcctggaactctggcgctctgacaagcggcgtgcacacctttccagccgtgctgcagagcagcggcctgtactctctgagcagcgtcgtgacagtgcccagcagctctctgggcacccagacctacatctgcaacgtgaaccacaagcccagcaacaccaaggtggacaagaaggtggaacccaagagctgcgacaagacccacacctgtcccccttgtcctgcccccgaactgctgggaggcccttccgtgttcctgttccccccaaagcccaaggacaccctgatgatcagccggacccccgaagtgacctgcgtggtggtggatgtgtcccacgaggaccctgaagtgaagttcaattggtacgtggacggcgtggaagtgcacaacgccaagaccaagcctagagaggaacagtacaacagcacctaccgggtggtgtccgtgctgacagtgctgcaccaggactggctgaacggcaaagagtacaagtgcaaggtgtccaacaaggccctgcctgcccccatcgagaaaaccatcagcaaggccaagggccagccccgcgaaccccaggtgtacacactgcccccaagcagggacgagctgaccaagaaccaggtgtccctgacctgtctcgtgaaaggcttctacccctccgatatcgccgtggaatgggagagcaacggccagcccgagaacaactacaagaccaccccccctgtgctggacagcgacggctcattcttcctgtacagcaagctgaccgtggacaagtcccggtggcagcagggcaacgtgttcagctgcagcgtgatgcacgaggccctgcacaaccactacacccagaagtccctgagcctgagccctggcaag

SEQ ID NO: 500 - DNA, 651 bp, synthetic construct; identical to SEQ ID NO: 440
  misc_feature 1..651: note = antibody sequence
  source 1..651: mol_type = other DNA

SEQ ID NO: 501 - protein, 123 aa, synthetic construct; identical to SEQ ID NO: 381
  REGION 1..123: note = antibody sequence

SEQ ID NO: 502 - protein, 5 aa, synthetic construct
  REGION 1..5: note = antibody sequence
SYEMN

SEQ ID NO: 503 - protein, 17 aa, synthetic construct
  REGION 1..17: note = antibody sequence
GISWNSGWIDYADSVKG

SEQ ID NO: 504 - protein, 14 aa, synthetic construct
  REGION 1..14: note = antibody sequence
SGYSSSWFDPDFDY

SEQ ID NO: 505 - protein, 111 aa, synthetic construct
  REGION 1..111: note = antibody sequence
QSVLTQPPSVSGAPGQRVTISCTGSSSDIGAGYDVHWYQQLPGTAPKLLIYGNSNRPSGVPDRFSGSKSGTSASLAITGLQAEDEADYYCQSYAGINPYVVFGGGTKLTVL

SEQ ID NO: 506 - protein, 14 aa, synthetic construct
  REGION 1..14: note = antibody sequence
TGSSSDIGAGYDVH

SEQ ID NO: 507 - protein, 7 aa, synthetic construct
  REGION 1..7: note = antibody sequence
GNSNRPS

SEQ ID NO: 508 - protein, 11 aa, synthetic construct
  REGION 1..11: note = antibody sequence
QSYAGINPYVV

SEQ ID NO: 509 - DNA, 369 bp, synthetic construct; identical to SEQ ID NO: 389
  misc_feature 1..369: note = antibody sequence
  source 1..369: mol_type = other DNA

SEQ ID NO: 510 - DNA, 15 bp, synthetic construct
  misc_feature 1..15: note = antibody sequence
  source 1..15: mol_type = other DNA
agctacgagatgaac

SEQ ID NO: 511 - DNA, 51 bp, synthetic construct; identical to SEQ ID NO: 391
  misc_feature 1..51: note = antibody sequence
  source 1..51: mol_type = other DNA

SEQ ID NO: 512 - DNA, 42 bp, synthetic construct; identical to SEQ ID NO: 72
  misc_feature 1..42: note = antibody sequence
  source 1..42: mol_type = other DNA

SEQ ID NO: 513 - DNA, 333 bp, synthetic construct
  misc_feature 1..333: note = antibody sequence
  source 1..333: mol_type = other DNA
cagtctgttctgacacagcctccatctgtgtctggcgcccctggacagagagtgaccatcagctgtacaggcagcagctccgatattggcgccggatacgacgtgcactggtatcagcaactgcctggcacagcccctaagctgctgatctacggcaacagcaacagacctagcggcgtgcccgatagattcagcggctctaagtctggcacaagcgccagcctggccattactggactgcaggccgaagatgaggccgactactactgtcagagctacgccggcatcaacccctacgtggtgtttggcggaggcaccaagctgacagttcta

SEQ ID NO: 514 - DNA, 42 bp, synthetic construct; identical to SEQ ID NO: 394
  misc_feature 1..42: note = antibody sequence
  source 1..42: mol_type = other DNA

SEQ ID NO: 515 - DNA, 21 bp, synthetic construct
  misc_feature 1..21: note = antibody sequence
  source 1..21: mol_type = other DNA
ggcaacagcaacagacctagc

SEQ ID NO: 516 - DNA, 33 bp, synthetic construct; identical to SEQ ID NO: 416
  misc_feature 1..33: note = antibody sequence
  source 1..33: mol_type = other DNA

SEQ ID NO: 517 - protein, 453 aa, synthetic construct; identical to SEQ ID NO: 397
  REGION 1..453: note = antibody sequence

SEQ ID NO: 518 - protein, 217 aa, synthetic construct
  REGION 1..217: note = antibody sequence
QSVLTQPPSVSGAPGQRVTISCTGSSSDIGAGYDVHWYQQLPGTAPKLLIYGNSNRPSGVPDRFSGSKSGTSASLAITGLQAEDEADYYCQSYAGINPYVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 519 - DNA, 1359 bp, synthetic construct; identical to SEQ ID NO: 399
  misc_feature 1..1359: note = antibody sequence
  source 1..1359: mol_type = other DNA

SEQ ID NO: 520 - DNA, 651 bp, synthetic construct
  misc_feature 1..651: note = antibody sequence
  source 1..651: mol_type = other DNA
cagtctgttctgacacagcctccatctgtgtctggcgcccctggacagagagtgaccatcagctgtacaggcagcagctccgatattggcgccggatacgacgtgcactggtatcagcaactgcctggcacagcccctaagctgctgatctacggcaacagcaacagacctagcggcgtgcccgatagattcagcggctctaagtctggcacaagcgccagcctggccattactggactgcaggccgaagatgaggccgactactactgtcagagctacgccggcatcaacccctacgtggtgtttggcggaggcaccaagctgacagttctaggccagcctaaagccgcccctagcgtgaccctgttccctccaagcagcgaggaactgcaggccaacaaggccaccctcgtgtgcctgatcagcgacttctatcctggcgccgtgaccgtggcctggaaggccgatagctctcctgtgaaggccggcgtggaaaccaccacccctagcaagcagagcaacaacaaatacgccgccagcagctacctgagcctgacccccgagcagtggaagtcccacagatcctacagctgccaagtgacccacgagggcagcaccgtggaaaagacagtggcccctaccgagtgcagc

SEQ ID NO: 521 - protein, 123 aa, synthetic construct; identical to SEQ ID NO: 481
  REGION 1..123: note = antibody sequence

SEQ ID NO: 522 - protein, 5 aa, synthetic construct
  REGION 1..5: note = antibody sequence
SYEMN

SEQ ID NO: 523 - protein, 17 aa, synthetic construct
  REGION 1..17: note = antibody sequence
GISWNSGWIDYADSVKG

SEQ ID NO: 524 - protein, 14 aa, synthetic construct
  REGION 1..14: note = antibody sequence
SGYSSSWFDPDFDY

SEQ ID NO: 525 - protein, 111 aa, synthetic construct
  REGION 1..111: note = antibody sequence
QSVLTQPPSVSGAPGQRVTISCTGSSSDIGAGYDVHWYQQLPGTAPKLLIYGNSNRPSGVPDRFSGSKSGTSASLAITGLQAEDEADYYCSSYEGINPYVVFGGGTKLTVL

SEQ ID NO: 526 - protein, 14 aa, synthetic construct
  REGION 1..14: note = antibody sequence
TGSSSDIGAGYDVH

SEQ ID NO: 527 - protein, 7 aa, synthetic construct
  REGION 1..7: note = antibody sequence
GNSNRPS

SEQ ID NO: 528 - protein, 11 aa, synthetic construct
  REGION 1..11: note = antibody sequence
SSYEGINPYVV

SEQ ID NO: 529 - DNA, 369 bp, synthetic construct; identical to SEQ ID NO: 489
  misc_feature 1..369: note = antibody sequence
  source 1..369: mol_type = other DNA

SEQ ID NO: 530 - DNA, 15 bp, synthetic construct
  misc_feature 1..15: note = antibody sequence
  source 1..15: mol_type = other DNA
agctacgagatgaac

SEQ ID NO: 531 - DNA, 51 bp, synthetic construct; identical to SEQ ID NO: 391
  misc_feature 1..51: note = antibody sequence
  source 1..51: mol_type = other DNA

SEQ ID NO: 532 - DNA, 42 bp, synthetic construct; identical to SEQ ID NO: 72
  misc_feature 1..42: note = antibody sequence
  source 1..42: mol_type = other DNA

SEQ ID NO: 533 - DNA, 333 bp, synthetic construct
  misc_feature 1..333: note = antibody sequence
  source 1..333: mol_type = other DNA
cagtctgttctgacacagcctccatctgtgtctggcgcccctggacagagagtgaccatcagctgtacaggcagcagctccgatattggcgccggatacgacgtgcactggtatcagcaactgcctggcacagcccctaagctgctgatctacggcaacagcaacagacctagcggcgtgcccgatagattcagcggctctaagtctggcacaagcgccagcctggccattactggactgcaggccgaagatgaggccgactactactgcagcagctacgagggcatcaacccctacgtggtgtttggcggcggaacaaagctgaccgttcta

SEQ ID NO: 534 - DNA, 42 bp, synthetic construct; identical to SEQ ID NO: 394
  misc_feature 1..42: note = antibody sequence
  source 1..42: mol_type = other DNA

SEQ ID NO: 535 - DNA, 21 bp, synthetic construct
  misc_feature 1..21: note = antibody sequence
  source 1..21: mol_type = other DNA
ggcaacagcaacagacctagc

SEQ ID NO: 536 - DNA, 33 bp, synthetic construct
  misc_feature 1..33: note = antibody sequence
  source 1..33: mol_type = other DNA
agcagctacgagggcatcaacccctacgtggtg

SEQ ID NO: 537 - protein, 453 aa, synthetic construct; identical to SEQ ID NO: 497
  REGION 1..453: note = antibody sequence

SEQ ID NO: 538 - protein, 217 aa, synthetic construct
  REGION 1..217: note = antibody sequence
QSVLTQPPSVSGAPGQRVTISCTGSSSDIGAGYDVHWYQQLPGTAPKLLIYGNSNRPSGVPDRFSGSKSGTSASLAITGLQAEDEADYYCSSYEGINPYVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 539 - DNA, 1359 bp, synthetic construct; identical to SEQ ID NO: 499
  misc_feature 1..1359: note = antibody sequence
  source 1..1359: mol_type = other DNA

SEQ ID NO: 540 - DNA, 651 bp, synthetic construct
  misc_feature 1..651: note = antibody sequence
  source 1..651: mol_type = other DNA
cagtctgttctgacacagcctccatctgtgtctggcgcccctggacagagagtgaccatcagctgtacaggcagcagctccgatattggcgccggatacgacgtgcactggtatcagcaactgcctggcacagcccctaagctgctgatctacggcaacagcaacagacctagcggcgtgcccgatagattcagcggctctaagtctggcacaagcgccagcctggccattactggactgcaggccgaagatgaggccgactactactgcagcagctacgagggcatcaacccctacgtggtgtttggcggcggaacaaagctgaccgttctaggccagcctaaagccgcccctagcgtgaccctgttccctccaagcagcgaggaactgcaggccaacaaggccaccctcgtgtgcctgatcagcgacttctatcctggcgccgtgaccgtggcctggaaggccgatagctctcctgtgaaggccggcgtggaaaccaccacccctagcaagcagagcaacaacaaatacgccgccagcagctacctgagcctgacccccgagcagtggaagtcccacagatcctacagctgccaagtgacccacgagggcagcaccgtggaaaagacagtggcccctaccgagtgcagc

SEQ ID NO: 541 - protein, 123 aa, synthetic construct; identical to SEQ ID NO: 441
  REGION 1..123: note = antibody sequence

SEQ ID NO: 542 - protein, 5 aa, synthetic construct
  REGION 1..5: note = antibody sequence
SYEMN

SEQ ID NO: 543 - protein, 17 aa, synthetic construct
  REGION 1..17: note = antibody sequence
GISWNSGWIDYADSVKG

SEQ ID NO: 544 - protein, 14 aa, synthetic construct
  REGION 1..14: note = antibody sequence
SGYSSSWFDPDFDY

SEQ ID NO: 545 - protein, 111 aa, synthetic construct
  REGION 1..111: note = antibody sequence
QSVLTQPPSVSGAPGQRVTISCTGSSSNIGAGYDVHWYQQLPGTAPKLLIYGASNRPSGVPDRFSGSKSGTSASLAITGLQAEDEADYYCSSYEGPNPYVVFGGGTKLTVL

SEQ ID NO: 546 - protein, 14 aa, synthetic construct
  REGION 1..14: note = antibody sequence
TGSSSNIGAGYDVH

SEQ ID NO: 547 - protein, 7 aa, synthetic construct
  REGION 1..7: note = antibody sequence
GASNRPS

SEQ ID NO: 548 - protein, 11 aa, synthetic construct
  REGION 1..11: note = antibody sequence
SSYEGPNPYVV

SEQ ID NO: 549 - DNA, 369 bp, synthetic construct; identical to SEQ ID NO: 449
  misc_feature 1..369: note = antibody sequence
  source 1..369: mol_type = other DNA

SEQ ID NO: 550 - DNA, 15 bp, synthetic construct
  misc_feature 1..15: note = antibody sequence
  source 1..15: mol_type = other DNA
agctacgagatgaac

SEQ ID NO: 551 - DNA, 51 bp, synthetic construct; identical to SEQ ID NO: 391
  misc_feature 1..51: note = antibody sequence
  source 1..51: mol_type = other DNA

SEQ ID NO: 552 - DNA, 42 bp, synthetic construct; identical to SEQ ID NO: 72
  misc_feature 1..42: note = antibody sequence
  source 1..42: mol_type = other DNA

SEQ ID NO: 553 - DNA, 333 bp, synthetic construct
  misc_feature 1..333: note = antibody sequence
  source 1..333: mol_type = other DNA
cagtctgttctgacacagcctccatctgtgtctggcgcccctggacagagagtgaccatcagctgtacaggcagcagctccaatatcggagccggctatgacgtgcactggtatcagcagctgcctggcacagcccctaaactgctgatctacggcgccagcaatagacctagcggcgtgcccgatagattcagcggctctaagtctggcacaagcgccagcctggccattactggactgcaggccgaagatgaggccgactactactgcagcagctacgagggccccaatccttacgtggtgtttggcggcggaacaaagctgaccgttcta

SEQ ID NO: 554 - DNA, 42 bp, synthetic construct; identical to SEQ ID NO: 114
  misc_feature 1..42: note = antibody sequence
  source 1..42: mol_type = other DNA

SEQ ID NO: 555 - DNA, 21 bp, synthetic construct
  misc_feature 1..21: note = antibody sequence
  source 1..21: mol_type = other DNA
ggcgccagcaatagacctagc

SEQ ID NO: 556 - DNA, 33 bp, synthetic construct
  misc_feature 1..33: note = antibody sequence
  source 1..33: mol_type = other DNA
agcagctacgagggccccaatccttacgtggtg

SEQ ID NO: 557 - protein, 453 aa, synthetic construct; identical to SEQ ID NO: 457
  REGION 1..453: note = antibody sequence

SEQ ID NO: 558 - protein, 217 aa, synthetic construct
  REGION 1..217: note = antibody sequence
QSVLTQPPSVSGAPGQRVTISCTGSSSNIGAGYDVHWYQQLPGTAPKLLIYGASNRPSGVPDRFSGSKSGTSASLAITGLQAEDEADYYCSSYEGPNPYVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 559 - DNA, 1359 bp, synthetic construct; identical to SEQ ID NO: 459
  misc_feature 1..1359: note = antibody sequence
  source 1..1359: mol_type = other DNA

SEQ ID NO: 560 - DNA, 651 bp, synthetic construct
  misc_feature 1..651: note = antibody sequence
  source 1..651: mol_type = other DNA
cagtctgttctgacacagcctccatctgtgtctggcgcccctggacagagagtgaccatcagctgtacaggcagcagctccaatatcggagccggctatgacgtgcactggtatcagcagctgcctggcacagcccctaaactgctgatctacggcgccagcaatagacctagcggcgtgcccgatagattcagcggctctaagtctggcacaagcgccagcctggccattactggactgcaggccgaagatgaggccgactactactgcagcagctacgagggccccaatccttacgtggtgtttggcggcggaacaaagctgaccgttctaggccagcctaaagccgcccctagcgtgaccctgttccctccaagcagcgaggaactgcaggccaacaaggccaccctcgtgtgcctgatcagcgacttctatcctggcgccgtgaccgtggcctggaaggccgatagctctcctgtgaaggccggcgtggaaaccaccacccctagcaagcagagcaacaacaaatacgccgccagcagctacctgagcctgacccccgagcagtggaagtcccacagatcctacagctgccaagtgacccacgagggcagcaccgtggaaaagacagtggcccctaccgagtgcagc

SEQ ID NO: 561 - protein, 123 aa, synthetic construct; identical to SEQ ID NO: 381
  REGION 1..123: note = antibody sequence

SEQ ID NO: 562 - protein, 5 aa, synthetic construct
  REGION 1..5: note = antibody sequence
SYEMN

SEQ ID NO: 563 - protein, 17 aa, synthetic construct
  REGION 1..17: note = antibody sequence
GISWNSGWIDYADSVKG

SEQ ID NO: 564 - protein, 14 aa, synthetic construct
  REGION 1..14: note = antibody sequence
SGYSSSWFDPDFDY

SEQ ID NO: 565 - protein, 111 aa, synthetic construct
  REGION 1..111: note = antibody sequence
QSVLTQPPSVSGAPGQRVTISCTGSSSNIGAGYDVHWYQQLPGTAPKLLIYGNSNRPSGVPDRFSGSKSGTSASLAITGLQAEDEADYYCSSYAGPNPYVVFGGGTKLTVL

SEQ ID NO: 566 - protein, 14 aa, synthetic construct
  REGION 1..14: note = antibody sequence
TGSSSNIGAGYDVH

SEQ ID NO: 567 - protein, 7 aa, synthetic construct
  REGION 1..7: note = antibody sequence
GNSNRPS

SEQ ID NO: 568 - protein, 11 aa, synthetic construct
  REGION 1..11: note = antibody sequence
SSYAGPNPYVV

SEQ ID NO: 569 - DNA, 369 bp, synthetic construct; identical to SEQ ID NO: 389
  misc_feature 1..369: note = antibody sequence
  source 1..369: mol_type = other DNA

SEQ ID NO: 570 - DNA, 15 bp, synthetic construct
  misc_feature 1..15: note = antibody sequence
  source 1..15: mol_type = other DNA
agctacgagatgaac

SEQ ID NO: 571 - DNA, 51 bp, synthetic construct; identical to SEQ ID NO: 391
  misc_feature 1..51: note = antibody sequence
  source 1..51: mol_type = other DNA

SEQ ID NO: 572 - DNA, 42 bp, synthetic construct; identical to SEQ ID NO: 72
  misc_feature 1..42: note = antibody sequence
  source 1..42: mol_type = other DNA

SEQ ID NO: 573 - DNA, 333 bp, synthetic construct
  misc_feature 1..333: note = antibody sequence
  source 1..333: mol_type = other DNA
cagtctgttctgacacagcctccatctgtgtctggcgcccctggacagagagtgaccatcagctgtacaggcagcagctccaatatcggagccggctatgacgtgcactggtatcagcagctgcctggcacagcccctaaactgctgatctacggcaacagcaacagacccagcggcgtgcccgatagattttccggctctaagagcggcacaagcgccagcctggctattactggactgcaggccgaggacgaggccgactactactgtagctcttacgctggccccaatccttacgtggtgtttggcggcggaacaaagctgaccgttcta

SEQ ID NO: 574 - DNA, 42 bp, synthetic construct; identical to SEQ ID NO: 114
  misc_feature 1..42: note = antibody sequence
  source 1..42: mol_type = other DNA

SEQ ID NO: 575 - DNA, 21 bp, synthetic construct
  misc_feature 1..21: note = antibody sequence
  source 1..21: mol_type = other DNA
ggcaacagcaacagacccagc

SEQ ID NO: 576 - DNA, 33 bp, synthetic construct
  misc_feature 1..33: note = antibody sequence
  source 1..33: mol_type = other DNA
agctcttacgctggccccaatccttacgtggtg

SEQ ID NO: 577 - protein, 453 aa, synthetic construct; identical to SEQ ID NO: 397
  REGION 1..453: note = antibody sequence

SEQ ID NO: 578 - protein, 217 aa, synthetic construct
  REGION 1..217: note = antibody sequence
QSVLTQPPSVSGAPGQRVTISCTGSSSNIGAGYDVHWYQQLPGTAPKLLIYGNSNRPSGVPDRFSGSKSGTSASLAITGLQAEDEADYYCSSYAGPNPYVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 579 - DNA, 1359 bp, synthetic construct; identical to SEQ ID NO: 399
  misc_feature 1..1359: note = antibody sequence
  source 1..1359: mol_type = other DNA

SEQ ID NO: 580 - DNA, 651 bp, synthetic construct
  misc_feature 1..651: note = antibody sequence
  source 1..651: mol_type = other DNA
cagtctgttctgacacagcctccatctgtgtctggcgcccctggacagagagtgaccatcagctgtacaggcagcagctccaatatcggagccggctatgacgtgcactggtatcagcagctgcctggcacagcccctaaactgctgatctacggcaacagcaacagacccagcggcgtgcccgatagattttccggctctaagagcggcacaagcgccagcctggctattactggactgcaggccgaggacgaggccgactactactgtagctcttacgctggccccaatccttacgtggtgtttggcggcggaacaaagctgaccgttctaggccagcctaaagccgcccctagcgtgaccctgttccctccaagcagcgaggaactgcaggccaacaaggccaccctcgtgtgcctgatcagcgacttctatcctggcgccgtgaccgtggcctggaaggccgatagctctcctgtgaaggccggcgtggaaaccaccacccctagcaagcagagcaacaacaaatacgccgccagcagctacctgagcctgacccccgagcagtggaagtcccacagatcctacagctgccaagtgacccacgagggcagcaccgtggaaaagacagtggcccctaccgagtgcagc

SEQ ID NO: 581 - protein, 990 aa, Homo sapiens
HHHHHHKNNVPRLKLSYKEMLESNNVITFNGLANSSSYHTFLLDEERSRLYVGAKDHIFSFDLVNIKDFQKIVWPVSYTRRDECKWAGKDILKECANFIKVLKAYNQTHLYACGTGAFHPICTYIEIGHHPEDNIFKLENSHFENGRGKSPYDPKLLTASLLIDGELYSGTAADFMGRDFAIFRTLGHHHPIRTEQHDSRWLNDPKFISAHLISESDNPEDDKVYFFFRENAIDGEHSGKATHARIGQICKNDFGGHRSLVNKWTTFLKARLICSVPGPNGIDTHFDELQDVFLMNFKDPKNPVVYGVFTTSSNIFKGSAVCMYSMSDVRRVFLGPYAHRDGPNYQWVPYQGRVPYPRPGTCPSKTFGGFDSTKDLPDDVITFARSHPAMYNPVFPMNNRPIVIKTDVNYQFTQIVVDRVDAEDGQYDVMFIGTDVGTVLKVVSIPKETWYDLEEVLLEEMTVFREPTAISAMELSTKQQQLYIGSTAGVAQLPLHRCDIYGKACAECCLARDPYCAWDGSACSRYFPTAKRATRAQDIRNGDPLTHCSDLHHDNHHGHSPEERIIYGVENSSTFLECSPKSQRALVYWQFQRRNEERKEEIRVDDHIIRTDQGLLLRSLQQKDSGNYLCHAVEHGFIQTLLKVTLEVIDTEHLEELLHKDDDGDGSKTKEMSNSMTPSQKVWYRDFMQLINHPNLNTMDEFCEQVWKRDRKQRRQRPGHTPGNSNKWKHLQENKKGRNRRTHEFERAPRSVDIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 582 - protein, 563 aa, Homo sapiens
NYQNGKNNVPRLKLSYKEMLESNNVITFNGLANSSSYHTFLLDEERSRLYVGAKDHIFSFDLVNIKDFQKIVWPVSYTRRDECKWAGKDILKECANFIKVLKAYNQTHLYACGTGAFHPICTYIEIGHHPEDNIFKLENSHFENGRGKSPYDPKLLTASLLIDGELYSGTAADFMGRDFAIFRTLGHHHPIRTEQHDSRWLNDPKFISAHLISESDNPEDDKVYFFFRENAIDGEHSGKATHARIGQICKNDFGGHRSLVNKWTTFLKARLICSVPGPNGIDTHFDELQDVFLMNFKDPKNPVVYGVFTTSSNIFKGSAVCMYSMSDVRRVFLGPYAHRDGPNYQWVPYQGRVPYPRPGTCPSKTFGGFDSTKDLPDDVITFARSHPAMYNPVFPMNNRPIVIKTDVNYQFTQIVVDRVDAEDGQYDVMFIGTDVGTVLKVVSIPKETWYDLEEVLLEEMTVFREPTAISAMELSTKQQQLYIGSTAGVAQLPLHRCDIYGKACAECCLARDPYCAWDGSACSRYFPTAKARTRAQDIRNGDPLTHCSDGGIEGRMDHHHHHH

SEQ ID NO: 583 - protein, 563 aa, Mus musculus
NYANGKNNVPRLKLSYKEMLESNNVITFNGLANSSSYHTFLLDEERSRLYVGAKDHIFSFNLVNIKDFQKIVWPVSYTRRDECKWAGKDILKECANFIKVLEAYNQTHLYACGTGAFHPICTYIEVGHHPEDNIFKLQDSHFENGRGKSPYDPKLLTASLLIDGELYSGTAADFMGRDFAIFRTLGHHHPIRTEQHDSRWLNDPRFISAHLIPESDNPEDDKVYFFFRENAIDGEHSGKATHARIGQICKNDFGGHRSLVNKWTTFLKARLICSVPGPNGIDTHFDELQDVFLMNSKDPKNPIVYGVFTTSSNIFKGSAVCMYSMSDVRRVFLGPYAHRDGPNYQWVPYQGRVPYPRPGTCPSKTFGGFDSTKDLPDDVITFARSHPAMYNPVFPINNRPIMIKTDVNYQFTQIVVDRVDAEDGQYDVMFIGTDVGTVLKVVSVPKETWHDLEEILLEEMTVFREPTTISAMELSTKQQQLYIGSTAGVAQLPLHRCDIYGKACAECCLARDPYCAWDGSSCSRYFPTAKARTRAQDIRNGDPLTHCSDGGIEGRMDHHHHHH

SEQ ID NO: 584 - protein, 563 aa, Rattus norvegicus
NYANGKNNVPRLKLSYKEMLESNNVITFNGLANSSSYHTFLLDEERSRLYVGAKDHIFSFNLVNIKDFQKIVWPVSYTRRDECKWAGKDILKECANFIKVLKAYNQTHLYACGTGAFHPICTYIEVGHHPEDNIFKLQDSHFENGRGKSPYDPKLLTASLLIDGELYSGTAADFMGRDFAIFRTLGHHHPIRTEQHDSRWLNDPRFISAHLIPESDNPEDDKVYFFFRENAIDGEHSGKATHARIGQICKNDFGGHRSLVNKWTTFLKARLICSVPGPNGIDTHFDELQDVFLMNSKDPKNPIVYGVFTTSSNIFKGSAVCMYSMSDVRRVFLGPYAHRDGPNYQWVPYQGRVPYPRPGTCPSKTFGGFDSTKDLPDDVITFARSHPAMYNPVFPINNRPIMIKTDVNYQFTQIVVDRVDAEDGQYDVMFIGTDVGTVLKVVSVPKETWHDLEEVLLEEMTVFREPTTISAMELSTKQQQLYIGSTAGVAQLPLHRCDIYGKACAECCLARDPYCAWDGSSCSRYFPTAKARTRAQDIRNGDPLTHCSDGGIEGRMDHHHHHH

SEQ ID NO: 585 - protein, 563 aa, Canis lupus
NYQNGKNNVPRLKLSYKEMLESNSVITFNGLANSSSYHTFLLDEERSRLYVGAKDHIFSFNLVNIKDFQKIVWPVSYTRRDECKWAGKDIQKECANFIKVLKAYNQTHLYACGTGAFHPICTYIEIGHHPEDNIFKLEDSHFENGRGKSPYDPKLLTASLLIDGELYSGTAADFMGRDFAIFRTLGHHHPIRTEQHDSRWLNDPRFISAHLIPESDNPEDDKVYFFFRENAIDGEHTGKATHARIGQICKNDFGGHRSLVNKWTTFLKARLICSVPGPNGIDTHFDELQDVFLMNSKDPKNPIVYGVFTTSSNIFKGSAVCMYSMSDVRRVFLGPYAHRDGPNYQWVPYQGRVPYPRPGTCPSKTFGGFDSTKDLPDDVITFARSHPAMYNPVFPINNRPIMIKTDVNYQFTQIVVDRVDAEDGQYDVMFIGTDVGTVLKVVSIPKETWHDLEEVLLEEMTVFREPTPISAMELSTKQHQLYAGSPAGLAQLPLQRCAAYGRACAECCLARDPYCAWDGAACSRYFPAAKARTRAQDIRNGDPLTHCSDGGIEGRMDHHHHHH

SEQ ID NO: 586 - protein, 563 aa, Macaca fascicularis
NYQNGKNNVPRLKLSYKEMLESNNVITFNGLANSSSYHTFLLDEERSRLYVGAKDHIFSFNLVNIKDFQKIVWPVSYTRRDECKWAGKDILKECANFIKVLKAYNQTHLYACGTGAFHPICTYIEIGHHPEDNIFKLENSHFENGRGKSPYDPKLLTASLLIDGELYSGTAADFMGRDFAIFRTLGHHHPIRTEQHDSRWLNDPRFISAHLIPESDNPEDDKVYFFFRENAIDGEHSGKATHARIGQICKNDFGGHRSLVNKWTTFLKARLICSVPGPNGIDTHFDELQDVFLMNFKDPKNPIVYGVFTTSSNIFKGSAVCMYSMSDVRRVFLGPYAHRDGPNYQWVPYQGRVPYPRPGTCPSKTFGGFDSTKDLPDDVITFARSHPAMYNPVFPINNRPIMIKTDVNYQFTQIVVDRVDAEDGQYDVMFIGTDVGTVLKVVSIPKETWHDLEEVLLEEMTVFREPTTISAMELSTKQQQLYIGSTAGIAQLPLHRCDIYGKACAECCLARDPYCAWDGSSCSRYFPTAKARTRAQDIRNGDPLTHCSDGGIEGRMDHHHHHH

SEQ ID NO: 587 - protein, 563 aa, Sus scrofa
NYQNGKNNVPRLKLSYKEMLESNNVITFNGLANSSSYHTFLLDEERSRLYVGAKDHIFSFNLVNIKDFQKIVWPVSYTRRDECKWAGKDILKECANFIKVLKAYNQTHLYACGTGAFHPICTYIEIGHHPEDNIFKLEDSHFENGRGKSPYDPKLLTASLLIDGELYSGTAADFMGRDFAIFRTLGHHHPIRTEQHDSRWLNDPRFISAHLIPESDNPEDDKVYFFFRENAIDGEHTGKATHARIGQICKNDFGGHRSLVNKWTTFLKARLICSVPGPNGIDTHFDELQDVFLMNSKDPKNPVVYGVFTTSSNIFKGSAVCMYSMSDVRRVFLGPYAHRDGPNYQWVPYQGRVPYPRPGTCPSKTFGGFDSTKDLPDDVITFARSHPAMYNPVFPINNRPIMIKTDVNYQFTQIVVDRVDAEDGQYDVMFIGTDVGTVLKVVSIPKETWHDLEEVLLEEMTVFREPTTISAMELSTKQQQLYVGSAAGVAQLPLHRCDIYGKACAECCLARDPYCAWDGSSCSRYFPTAKARTRAQDIRNGDPLTHCSDGGIEGRMDHHHHHH

SEQ ID NO: 588 - skipped

SEQ ID NO: 589 - protein, 5 aa, synthetic construct
  REGION 1..5: note = antibody sequence
  SITE 5: note = site - Xaa can be any naturally occurring amino acid
SYAMX

SEQ ID NO: 590 - protein, 16 aa, synthetic construct
  REGION 1..16: note = antibody sequence
  SITE 4: note = site - Xaa can be any naturally occurring amino acid
  SITE 15: note = site - Xaa can be any naturally occurring amino acid
AIGXGGDTYYADSVXG

SEQ ID NO: 591 - protein, 16 aa, synthetic construct
  REGION 1..16: note = antibody sequence
  SITE 4: note = site - Xaa can be any naturally occurring amino acid
AIGXGGDTYYADSVKG

SEQ ID NO: 592 - protein, 17 aa, synthetic construct
  REGION 1..17: note = antibody sequence
  SITE 8: note = site - Xaa can be any naturally occurring amino acid
  SITE 10: note = site - Xaa can be any naturally occurring amino acid
GISWNSGXIXYADSVKG

SEQ ID NO: 593 - protein, 17 aa, synthetic construct
  REGION 1..17: note = antibody sequence
  SITE 10: note = site - Xaa can be any naturally occurring amino acid
GISWNSGWIXYADSVKG

SEQ ID NO: 594 - protein, 12 aa, synthetic construct
  REGION 1..12: note = antibody sequence
  SITE 12: note = site - Xaa can be any naturally occurring amino acid
RDDYTSRDAFDX

SEQ ID NO: 595 - protein, 14 aa, synthetic construct
  REGION 1..14: note = antibody sequence
  SITE 6: note = site - Xaa can be any naturally occurring amino acid
TGSSSXIGAGYDVH

SEQ ID NO: 596 - protein, 7 aa, synthetic construct
  SITE 5: note = site - Xaa can be any naturally occurring amino acid
YDDLXPS

SEQ ID NO: 597 - protein, 7 aa, synthetic construct
  REGION 1..7: note = antibody sequence
  SITE 2: note = site - Xaa can be any naturally occurring amino acid
GXSNRPS

SEQ ID NO: 598 - protein, 12 aa, synthetic construct
  REGION 1..12: note = antibody sequence
  SITE 1: note = site - Xaa can be any naturally occurring amino acid
  REGION 9..11: note = site - Xaa can be any naturally occurring amino acid
XAWDDSLNXXXV

SEQ ID NO: 599 - protein, 11 aa, synthetic construct
  REGION 1..11: note = antibody sequence
  SITE 1: note = site - Xaa can be any naturally occurring amino acid
  SITE 4: note = site - Xaa can be any naturally occurring amino acid
  SITE 6: note = site - Xaa can be any naturally occurring amino acid
XSYXGXNPYVV

SEQ ID NO: 600 - protein, 750 aa, Homo sapiens
YQNGKNNVPRLKLSYKEMLESNNVITFNGLANSSSYHTFLLDEERSRLYVGAKDHIFSFDLVNIKDFQKIVWPVSYTRRDECKWAGKDILKECANFIKVLKAYNQTHLYACGTGAFHPICTYIEIGHHPEDNIFKLENSHFENGRGKSPYDPKLLTASLLIDGELYSGTAADFMGRDFAIFRTLGHHHPIRTEQHDSRWLNDPKFISAHLISESDNPEDDKVYFFFRENAIDGEHSGKATHARIGQICKNDFGGHRSLVNKWTTFLKARLICSVPGPNGIDTHFDELQDVFLMNFKDPKNPVVYGVFTTSSNIFKGSAVCMYSMSDVRRVFLGPYAHRDGPNYQWVPYQGRVPYPRPGTCPSKTFGGFDSTKDLPDDVITFARSHPAMYNPVFPMNNRPIVIKTDVNYQFTQIVVDRVDAEDGQYDVMFIGTDVGTVLKVVSIPKETWYDLEEVLLEEMTVFREPTAISAMELSTKQQQLYIGSTAGVAQLPLHRCDIYGKACAECCLARDPYCAWDGSACSRYFPTAKRRTRRQDIRNGDPLTHCSDLHHDNHHGHSPEERIIYGVENSSTFLECSPKSQRALVYWQFQRRNEERKEEIRVDDHIIRTDQGLLLRSLQQKDSGNYLCHAVEHGFIQTLLKVTLEVIDTEHLEELLHKDDDGDGSKTKEMSNSMTPSQKVWYRDFMQLINHPNLNTMDEFCEQVWKRDRKQRRQRPGHTPGNSNKWKHLQENKKGRNRRTHEFERAPRSV

SEQ ID NO: 601 - protein, 752 aa, Mus musculus
NYANGKNNVPRLKLSYKEMLESNNVITFNGLANSSSYHTFLLDEERSRLYVGAKDHIFSFNLVNIKDFQKIVWPVSYTRRDECKWAGKDILKECANFIKVLEAYNQTHLYACGTGAFHPICTYIEVGHHPEDNIFKLQDSHFENGRGKSPYDPKLLTASLLIDGELYSGTAADFMGRDFAIFRTLGHHHPIRTEQHDSRWLNDPRFISAHLIPESDNPEDDKVYFFFRENAIDGEHSGKATHARIGQICKNDFGGHRSLVNKWTTFLKARLICSVPGPNGIDTHFDELQDVFLMNSKDPKNPIVYGVFTTSSNIFKGSAVCMYSMSDVRRVFLGPYAHRDGPNYQWVPYQGRVPYPRPGTCPSKTFGGFDSTKDLPDDVITFARSHPAMYNPVFPINNRPIMIKTDVNYQFTQIVVDRVDAEDGQYDVMFIGTDVGTVLKVVSVPKETWHDLEEVLLEEMTVFREPTTISAMELSTKQQQLYIGSTAGVAQLPLHRCDIYGKACAECCLARDPYCAWDGSSCSRYFPTAKRRTRRQDIRNGDPLTHCSDLQHHDNHHGPSLEERIIYGVENSSTFLECSPKSQRALVYWQFQRRNEDRKEEIRMGDHIIRTEQGLLLRSLQKKDSGNYLCHAVEHGFMQTLLKVTLEVIDTEHLEELLHKDDDGDGSKIKEMSSSMTPSQKVWYRDFMQLINHPNLNTMDEFCEQVWKRDRKQRRQRPGHSQGSSNKWKHMQESKKGRNRRTHEFERAPRSV

SEQ ID NO: 602 - protein, 772 aa, Macaca fascicularis
MGWLTRIVCLFWGVLLTARANYQNGKNNVPRLKLSYKEMLESNNVITFNGLANSSSYHTFLLDEERSRLYVGAKDHIFSFNLVNIKDFQKIVWPVSYTRRDECKWAGKDILKECANFIKVLKAYNQTHLYACGTGAFHPICTYIEIGHHPEDNIFKLENSHFENGRGKSPYDPKLLTASLLIDGELYSGTAADFMGRDFAIFRTLGHHHPIRTEQHDSRWLNDPRFISAHLIPESDNPEDDKVYFFFRENAIDGEHSGKATHARIGQICKNDFGGHRSLVNKWTTFLKARLICSVPGPNGIDTHFDELQDVFLMNFKDPKNPIVYGVFTTSSNIFKGSAVCMYSMSDVRRVFLGPYAHRDGPNYQWVPYQGRVPYPRPGTCPSKTFGGFDSTKDLPDDVITFARSHPAMYNPVFPINNRPIMIKTDVNYQFTQIVVDRVDAEDGQYDVMFIGTDVGTVLKVVSIPKETWHDLEEVLLEEMTVFREPTTISAMELSTKQQQLYIGSTAGIAQLPLHRCDIYGKACAECCLARDPYCAWDGSSCSRYFPTAKRRTRRQDIRNGDPLTHCSDLQHHDNHHGHSPEERIIYGVENSSTFLECSPKSQRALVYWQFQRRNEERKEEIRVDDHIIRTDQGLLLRSLQRKDSGSYLCHAVEHGFIQTLLKVTLEVIDTEHLEELLHKDDDGDGSKTKEMSNSMTPSQKVWYRDFMQLINHPNLNTMDEFCEQVWKRDRKQRRQRPGHTQGNSNKWKHLQENKKGRNRRTHEFERAPRSV

SEQ ID NO: 603 - protein, 752 aa, Rattus norvegicus
NYANGKNNVPRLKLSYKEMLESNNVITFNGLANSSSYHTFLLDEERSRLYVGAKDHIFSFNLVNIKDFQKIVWPVSYTRRDECKWAGKDILKECANFIKVLKAYNQTHLYACGTGAFHPICTYIEVGHHPEDNIFKLQDSHFENGRGKSPYDPKLLTASLLIDGELYSGTAADFMGRDFAIFRTLGHHHPIRTEQHDSRWLNDPRFISAHLIPESDNPEDDKVYFFFRENAIDGEHSGKATHARIGQICKNDFGGHRSLVNKWTTFLKARLICSVPGPNGIDTHFDELQDVFLMNSKDPKNPIVYGVFTTSSNIFKGSAVCMYSMSDVRRVFLGPYAHRDGPNYQWVPYQGRVPYPRPGTCPSKTFGGFDSTKDLPDDVITFARSHPAMYNPVFPINNRPIMIKTDVNYQFTQIVVDRVDAEDGQYDVMFIGTDVGTVLKVVSVPKETWHDLEEVLLEEMTVFREPTTISAMELSTKQQQLYIGSTAGVAQLPLHRCDIYGKACAECCLARDPYCAWDGSSCSRYFPTAKRRTRRQDIRNGDPLTHCSDLQHHDNHHGHSLEERIIYGVENSSTFLECSPKSQRALVYWQFQRRNEDRKEEIRVGDHIIRTEQGLLLRSLQKKDSGNYLCHAVEHGFMQTLLKVTLEVIDTEHLEELLHKDDDGDGSKTKEMSSSMTPSQKVWYRDFMQLINHPNLNTMDEFCEQVWKRDRKQRRQRPGHSQGSSNKWKHMQESKKGRNRRTHEFERAPRSV

SEQ ID NO: 604 - protein, 772 aa, Sus scrofa
MGWFSRIVCLFWGVLLTARANYQNGKNNVPRLKLSYKEMLESNNVITFNGLANSSSYHTFLLDEERSRLYVGAKDHIFSFNLVNIKDFQKIVWPVSYTRRDECKWAGKDILKECANFIKVLKAYNQTHLYACGTGAFHPICTYIEIGHHPEDNIFKLEDSHFENGRGKSPYDPKLLTASLLIDGELYSGTAADFMGRDFAIFRTLGHHHPIRTEQHDSRWLNDPRFISAHLIPESDNPEDDKVYFFFRENAIDGEHTGKATHARIGQICKNDFGGHRSLVNKWTTFLKARLICSVPGPNGIDTHFDELQDVFLMNSKDPKNPVVYGVFTTSSNIFKGSAVCMYSMSDVRRVFLGPYAHRDGPNYQWVPYQGRVPYPRPGTCPSKTFGGFDSTKDLPDDVITFARSHPAMYNPVFPINNRPIMIKTDVNYQFTQIVVDRVDAEDGQYDVMFIGTDVGTVLKVVSIPKETWHDLEEVLLEEMTVFREPTTISAMELSTKQQQLYVGSAAGVAQLPLHRCDIYGKACAECCLARDPYCAWDGSSCSRYFPTAKRRTRRQDIRNGDPLTHCSDLQHHDNHRGHNFEERIIYGVENSSTFLECSPKSQRALVYWQFQRRNEERKEEIRVDDHIIRTEQGLLLRSLQRKDSGSYLCHAVEHGFMQTLLKVTLEVIDTEHLEELLHKDDDGDSSKTKEMSNSMTPSQKIWYRDFMQLINHPNLNTMDEFCEQVWKRDRKQRRQRPGHTQGNSNKWKHLQENKKCRNRRTHEFERAPRSV

SEQ ID NO: 605 - protein, 772 aa, Canis lupus
MGWLARIACLFWGVLLTATANYQNGKNNVPRLKLSYKEMLESNSVITFNGLANSSSYHTFLLDEERSRLYVGAKDHIFSFNLVNIKDFQKIVWPVSYTRRDECKWAGKDIQKECANFIKVLKAYNQTHLYACGTGAFHPICTYIEIGHHPEDNIFKLEDSHFENGRGKSPYDPKLLTASLLIDGELYSGTAADFMGRDFAIFRTLGHHHPIRTEQHDSRWLNDPRFISAHLIPESDNPEDDKVYFFFRENAIDGEHTGKATHARIGQICKNDFGGHRSLVNKWTTFLKARLICSVPGPNGIDTHFDELQDVFLMNSKDPKNPIVYGVFTTSSNIFKGSAVCMYSMSDVRRVFLGPYAHRDGPNYQWVPYQGRVPYPRPGTCPSKTFGGFDSTKDLPDDVITFARSHPAMYNPVFPINNRPIMIKTDVNYQFTQIVVDRVDAEDGQYDVMFIGTDVGTVLKVVSIPKETWHDLEEVLLEEMTVFREPTPISAMELSTKQHQLYAGSPAGLAQLPLQRCAAYGRACAECCLARDPYCAWDGAACSRYFPAAKRRTRRQDIRNGDPLTHCSDLQHHDNHHSHSLEERIIYGVENSSTFLECSPKSQRALVYWQFQRRNEERKEEIRVDDHIIRTEQGLLLRSLQRKDSGNYLCHAVEHGFMQTLLKVTLEVIDTEHLEELLHKDDDGDGSKTKEISNSMTPSQKVWYRDFMQLINHPNLNTMDEFCEQVWKRDRKQRRQRPGHTQGNSNKWKHLQENKKGRNRRTHEFERAPRSV

SEQ ID NO: 606-799 - skipped (194 entries)

SEQ ID NO: 800 - protein, 117 aa, synthetic construct
  REGION 1..117: note = antibody sequence
EVQLVESGGGLVQPGGSLRLSCAASGFTFSSYYMSWVRQAPGKGLEWVSTIIKSGGYAYYPDSVKDRFTISRDNSKNTLYLQMSSLRAEDTAVYYCVRGGQGAMDYWGQGTTVTVSS

SEQ ID NO: 801 - protein, 5 aa, synthetic construct
  REGION 1..5: note = antibody sequence
SYYMS

SEQ ID NO: 802 - protein, 17 aa, synthetic construct
  REGION 1..17: note = antibody sequence
TIIKSGGYAYYPDSVKD

SEQ ID NO: 803 - protein, 8 aa, synthetic construct
  REGION 1..8: note = antibody sequence
GGQGAMDY

SEQ ID NO: 804 - protein, 107 aa, synthetic construct
  REGION 1..107: note = antibody sequence
EIVLTQSPATLSLSPGERATLSCRASQSIGDYLHWYQQKPGQAPRLLIKYASQSISGIPARFSGSGSGTDFTLTITSLEPEDFAVYYCQQGYSFPYTFGGGTKLEIK

SEQ ID NO: 805 - protein, 11 aa, synthetic construct
  REGION 1..11: note = antibody sequence
RASQSIGDYLH

SEQ ID NO: 806 - protein, 7 aa, synthetic construct
  REGION 1..7: note = antibody sequence
YASQSIS

SEQ ID NO: 807 - protein, 9 aa, synthetic construct
  REGION 1..9: note = antibody sequence
QQGYSFPYT

SEQ ID NO: 808 - DNA, 351 bp, synthetic construct
  misc_feature 1..351: note = antibody sequence
  source 1..351: mol_type = other DNA
gaagtgcagctggtggaatctggcggaggactggttcaacctggcggctctctgagactgtcttgtgccgccagcggcttcaccttcagcagctactacatgagctgggtccgacaggcccctggcaaaggacttgaatgggtgtccaccatcatcaagagcggcggctacgcctactatcccgacagcgtgaaggaccggttcaccatctccagagacaacagcaagaacaccctgtacctgcagatgagcagcctgagagccgaggataccgccgtgtactactgtgttagaggcggacagggcgccatggattattggggccagggaaccacagtgaccgtgtcatca

SEQ ID NO: 809 - DNA, 321 bp, synthetic construct
  misc_feature 1..321: note = antibody sequence
  source 1..321: mol_type = other DNA
gagattgtgctgacacagtctcccgccacactgtctcttagccctggcgaaagagccacactgagctgtagagccagccagagcatcggcgattacctgcactggtatcagcagaagcctggacaggcccctcggctgctgattaagtacgccagccagtccatcagcggcatccctgccagattttctggcagcggctctggcaccgatttcaccctgaccatcaccagcctggaacctgaggacttcgccgtgtactactgccagcagggctacagcttcccctacacatttggcggaggcaccaagctggaaatcaaa

SEQ ID NO: 810 - protein, 447 aa, synthetic construct
  REGION 1..447: note = antibody sequence
EVQLVESGGGLVQPGGSLRLSCAASGFTFSSYYMSWVRQAPGKGLEWVSTIIKSGGYAYYPDSVKDRFTISRDNSKNTLYLQMSSLRAEDTAVYYCVRGGQGAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 811 - protein, 214 aa, synthetic construct
  REGION 1..214: note = antibody sequence
EIVLTQSPATLSLSPGERATLSCRASQSIGDYLHWYQQKPGQAPRLLIKYASQSISGIPARFSGSGSGTDFTLTITSLEPEDFAVYYCQQGYSFPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 812 - DNA, 1341 bp, synthetic construct
  misc_feature 1..1341: note = antibody sequence
  source 1..1341: mol_type = other DNA
gaagtgcagctggtggaatctggcggaggactggttcaacctggcggctctctgagactgtcttgtgccgccagcggcttcaccttcagcagctactacatgagctgggtccgacaggcccctggcaaaggacttgaatgggtgtccaccatcatcaagagcggcggctacgcctactatcccgacagcgtgaaggaccggttcaccatctccagagacaacagcaagaacaccctgtacctgcagatgagcagcctgagagccgaggataccgccgtgtactactgtgttagaggcggacagggcgccatggattattggggccagggaaccacagtgaccgtgtcatcagccagcaccaagggccccagcgtgttccctctggcccctagcagcaagagcacatctggcggaacagccgccctgggctgcctcgtgaaggactactttcccgagcccgtgaccgtgtcctggaactctggcgctctgacaagcggcgtgcacacctttccagccgtgctgcagagcagcggcctgtactctctgagcagcgtcgtgacagtgcccagcagctctctgggcacccagacctacatctgcaacgtgaaccacaagcccagcaacaccaaggtggacaagaaggtggaacccaagagctgcgacaagacccacacctgtcccccttgtcctgcccccgaactgctgggaggcccttccgtgttcctgttccccccaaagcccaaggacaccctgatgatcagccggacccccgaagtgacctgcgtggtggtggatgtgtcccacgaggaccctgaagtgaagttcaattggtacgtggacggcgtggaagtgcacaacgccaagaccaagcctagagaggaacagtacaacagcacctaccgggtggtgtccgtgctgacagtgctgcaccaggactggctgaacggcaaagagtacaagtgcaaggtgtccaacaaggccctgcctgcccccatcgagaaaaccatcagcaaggccaagggccagccccgcgaaccccaggtgtacacactgcccccaagcagggacgagctgaccaagaaccaggtgtccctgacctgtctcgtgaaaggcttctacccctccgatatcgccgtggaatgggagagcaacggccagcccgagaacaactacaagaccaccccccctgtgctggacagcgacggctcattcttcctgtacagcaagctgaccgtggacaagtcccggtggcagcagggcaacgtgttcagctgcagcgtgatgcacgaggccctgcacaaccactacacccagaagtccctgagcctgagccctggcaag

SEQ ID NO: 813 - DNA, 642 bp, synthetic construct
  misc_feature 1..642: note = antibody sequence
  source 1..642: mol_type = other DNA
gagattgtgctgacacagtctcccgccacactgtctcttagccctggcgaaagagccacactgagctgtagagccagccagagcatcggcgattacctgcactggtatcagcagaagcctggacaggcccctcggctgctgattaagtacgccagccagtccatcagcggcatccctgccagattttctggcagcggctctggcaccgatttcaccctgaccatcaccagcctggaacctgaggacttcgccgtgtactactgccagcagggctacagcttcccctacacatttggcggaggcaccaagctggaaatcaaacgaaccgtggccgctcccagcgtgttcatcttcccacctagcgacgagcagctgaagtccggcacagcctctgtcgtgtgcctgctgaacaacttctacccccgcgaggccaaggtgcagtggaaggtggacaatgccctgcagagcggcaacagccaggaaagcgtgaccgagcaggacagcaaggactccacctacagcctgagcagcaccctgaccctgagcaaggccgactacgagaagcacaaggtgtacgcctgcgaagtgacccaccagggcctgtctagccccgtgaccaagagcttcaaccggggcgagtgt

SEQ ID NO: 814 - protein, 117 aa, synthetic construct
  REGION 1..117: note = antibody sequence
EVQLVESGGGLVQPGGSLRLSCAASGFPFSSYYMSWVRQAPGKGLEWVSTIIKSGGYAYYPDSVKDRFTISRDNSKNTLYLQMSSLRAEDTAVYYCVRGGQGAMDYWGQGTTVTVSS

SEQ ID NO: 815 - protein, 5 aa, synthetic construct
  REGION 1..5: note = antibody sequence
SYYMS

SEQ ID NO: 816 - protein, 17 aa, synthetic construct
  REGION 1..17: note = antibody sequence
TIIKSGGYAYYPDSVKD

SEQ ID NO: 817 - protein, 8 aa, synthetic construct
  REGION 1..8: note = antibody sequence
GGQGAMDY

SEQ ID NO: 818 - protein, 107 aa, synthetic construct; identical to SEQ ID NO: 804
  REGION 1..107: note = antibody sequence

SEQ ID NO: 819 - protein, 11 aa, synthetic construct
  REGION 1..11: note = antibody sequence
RASQSIGDYLH

SEQ ID NO: 820 - protein, 7 aa, synthetic construct
  REGION 1..7: note = antibody sequence
YASQSIS

SEQ ID NO: 821 - protein, 9 aa, synthetic construct
  REGION 1..9: note = antibody sequence
QQGYSFPYT

SEQ ID NO: 822 - DNA, 351 bp, synthetic construct
  misc_feature 1..351: note = antibody sequence
  source 1..351: mol_type = other DNA
gaagtgcagctggtggaatctggcggaggactggttcaacctggcggctctctgagactgtcttgtgccgcctctggcttcccattcagcagctactacatgagctgggtccgacaggcccctggcaaaggacttgaatgggtgtccaccatcatcaagagcggcggctacgcctactatcccgacagcgtgaaggaccggttcaccatcagccgggacaacagcaagaacaccctgtacctgcagatgagcagcctgagagccgaggataccgccgtgtactactgtgttagaggcggacagggcgccatggattattggggccagggaaccacagtgaccgtgtcatca

SEQ ID NO: 823 - DNA, 321 bp, synthetic construct; identical to SEQ ID NO: 809
  misc_feature 1..321: note = antibody sequence
  source 1..321: mol_type = other DNA

SEQ ID NO: 824 - protein, 447 aa, synthetic construct
  REGION 1..447: note = antibody sequence
EVQLVESGGGLVQPGGSLRLSCAASGFPFSSYYMSWVRQAPGKGLEWVSTIIKSGGYAYYPDSVKDRFTISRDNSKNTLYLQMSSLRAEDTAVYYCVRGGQGAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 825 - protein, 214 aa, synthetic construct; identical to SEQ ID NO: 811
  REGION 1..214: note = antibody sequence

SEQ ID NO: 826 - DNA, 1341 bp, synthetic construct
  misc_feature 1..1341: note = antibody sequence
  source 1..1341: mol_type = other DNA
gaagtgcagctggtggaatctggcggaggactggttcaacctggcggctctctgagactgtcttgtgccgcctctggcttcccattcagcagctactacatgagctgggtccgacaggcccctggcaaaggacttgaatgggtgtccaccatcatcaagagcggcggctacgcctactatcccgacagcgtgaaggaccggttcaccatcagccgggacaacagcaagaacaccctgtacctgcagatgagcagcctgagagccgaggataccgccgtgtactactgtgttagaggcggacagggcgccatggattattggggccagggaaccacagtgaccgtgtcatcagccagcaccaagggccccagcgtgttccctctggcccctagcagcaagagcacatctggcggaacagccgccctgggctgcctcgtgaaggactactttcccgagcccgtgaccgtgtcctggaactctggcgctctgacaagcggcgtgcacacctttccagccgtgctgcagagcagcggcctgtactctctgagcagcgtcgtgacagtgcccagcagctctctgggcacccagacctacatctgcaacgtgaaccacaagcccagcaacaccaaggtggacaagaaggtggaacccaagagctgcgacaagacccacacctgtcccccttgtcctgcccccgaactgctgggaggcccttccgtgttcctgttccccccaaagcccaaggacaccctgatgatcagccggacccccgaagtgacctgcgtggtggtggatgtgtcccacgaggaccctgaagtgaagttcaattggtacgtggacggcgtggaagtgcacaacgccaagaccaagcctagagaggaacagtacaacagcacctaccgggtggtgtccgtgctgacagtgctgcaccaggactggctgaacggcaaagagtacaagtgcaaggtgtccaacaaggccctgcctgcccccatcgagaaaaccatcagcaaggccaagggccagccccgcgaaccccaggtgtacacactgcccccaagcagggacgagctgaccaagaaccaggtgtccctgacctgtctcgtgaaaggcttctacccctccgatatcgccgtggaatgggagagcaacggccagcccgagaacaactacaagaccaccccccctgtgctggacagcgacggctcattcttcctgtacagcaagctgaccgtggacaagtcccggtggcagcagggcaacgtgttcagctgcagcgtgatgcacgaggccctgcacaaccactacacccagaagtccctgagcctgagccctggcaag

SEQ ID NO: 827 - DNA, 642 bp, synthetic construct; identical to SEQ ID NO: 813
  misc_feature 1..642: note = antibody sequence
  source 1..642: mol_type = other DNA

SEQ ID NO: 828 - protein, 117 aa, synthetic construct
  REGION 1..117: note = antibody sequence
EVQLVESGGGLVQLGGSLRLSCAASGFTFSSYYMSWVRQAPGKGLEWVSTIIKSGGYAYYPDSVKDRFTISRDNSKNTLYLQMNSLRAEDTAVYYCVKGGQGAMDYWGQGTTVTVSS

SEQ ID NO: 829 - protein, 5 aa, synthetic construct
  REGION 1..5: note = antibody sequence
SYYMS

SEQ ID NO: 830 - protein, 17 aa, synthetic construct
  REGION 1..17: note = antibody sequence
TIIKSGGYAYYPDSVKD

SEQ ID NO: 831 - protein, 8 aa, synthetic construct
  REGION 1..8: note = antibody sequence
GGQGAMDY

SEQ ID NO: 832 - protein, 107 aa, synthetic construct
  REGION 1..107: note = antibody sequence
EIVLTQSPATLSLSPGERATLSCRASQSIGDYLHWYQQKPGQAPRLLIYYASQSISGIPARFSGSGSGTDFTLTISSLEPEDFAVYYCQQGYSFPYTFGGGTKLEIK

SEQ ID NO: 833 - protein, 11 aa, synthetic construct
  REGION 1..11: note = antibody sequence
RASQSIGDYLH

SEQ ID NO: 834 - protein, 7 aa, synthetic construct
  REGION 1..7: note = antibody sequence
YASQSIS

SEQ ID NO: 835 - protein, 9 aa, synthetic construct
  REGION 1..9: note = antibody sequence
QQGYSFPYT

SEQ ID NO: 836 - DNA, 351 bp, synthetic construct
  misc_feature 1..351: note = antibody sequence
  source 1..351: mol_type = other DNA
gaagtgcagctggtggaatctggcggaggactggttcagctcggcggatctctgagactgtcttgtgccgccagcggcttcaccttcagcagctactacatgagctgggtccgacaggcccctggcaaaggacttgaatgggtgtccaccatcatcaagagcggcggctacgcctactatcccgacagcgtgaaggaccggttcaccatctccagagacaacagcaagaacaccctgtacctgcagatgaacagcctgagagccgaggacaccgccgtgtactactgtgtgaaaggtggacagggcgccatggactattggggccagggaacaacagtgaccgtgtcctca

SEQ ID NO: 837 - DNA, 321 bp, synthetic construct
  misc_feature 1..321: note = antibody sequence
  source 1..321: mol_type = other DNA
gagattgtgctgacacagtctcccgccacactgtctcttagccctggcgaaagagccacactgagctgtagagccagccagagcatcggcgattacctgcactggtatcagcagaagcctggacaggcccctcggctgctgatctactatgccagccagtccatcagcggcatccccgccagattttctggcagcggctctggcaccgatttcaccctgaccataagcagcctggaacctgaggacttcgccgtgtactactgccagcagggctacagcttcccctacacatttggcggaggcaccaagctggaaatcaaa

SEQ ID NO: 838 - protein, 447 aa, synthetic construct
  REGION 1..447: note = antibody sequence
EVQLVESGGGLVQLGGSLRLSCAASGFTFSSYYMSWVRQAPGKGLEWVSTIIKSGGYAYYPDSVKDRFTISRDNSKNTLYLQMNSLRAEDTAVYYCVKGGQGAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 839 - protein, 214 aa, synthetic construct
  REGION 1..214: note = antibody sequence
EIVLTQSPATLSLSPGERATLSCRASQSIGDYLHWYQQKPGQAPRLLIYYASQSISGIPARFSGSGSGTDFTLTISSLEPEDFAVYYCQQGYSFPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 840 - DNA, 1341 bp, synthetic construct
  misc_feature 1..1341: note = antibody sequence
  source 1..1341: mol_type = other DNA
gaagtgcagctggtggaatctggcggaggactggttcagctcggcggatctctgagactgtcttgtgccgccagcggcttcaccttcagcagctactacatgagctgggtccgacaggcccctggcaaaggacttgaatgggtgtccaccatcatcaagagcggcggctacgcctactatcccgacagcgtgaaggaccggttcaccatctccagagacaacagcaagaacaccctgtacctgcagatgaacagcctgagagccgaggacaccgccgtgtactactgtgtgaaaggtggacagggcgccatggactattggggccagggaacaacagtgaccgtgtcctcagccagcaccaagggccccagcgtgttccctctggcccctagcagcaagagcacatctggcggaacagccgccctgggctgcctcgtgaaggactactttcccgagcccgtgaccgtgtcctggaactctggcgctctgacaagcggcgtgcacacctttccagccgtgctgcagagcagcggcctgtactctctgagcagcgtcgtgacagtgcccagcagctctctgggcacccagacctacatctgcaacgtgaaccacaagcccagcaacaccaaggtggacaagaaggtggaacccaagagctgcgacaagacccacacctgtcccccttgtcctgcccccgaactgctgggaggcccttccgtgttcctgttccccccaaagcccaaggacaccctgatgatcagccggacccccgaagtgacctgcgtggtggtggatgtgtcccacgaggaccctgaagtgaagttcaattggtacgtggacggcgtggaagtgcacaacgccaagaccaagcctagagaggaacagtacaacagcacctaccgggtggtgtccgtgctgacagtgctgcaccaggactggctgaacggcaaagagtacaagtgcaaggtgtccaacaaggccctgcctgcccccatcgagaaaaccatcagcaaggccaagggccagccccgcgaaccccaggtgtacacactgcccccaagcagggacgagctgaccaagaaccaggtgtccctgacctgtctcgtgaaaggcttctacccctccgatatcgccgtggaatgggagagcaacggccagcccgagaacaactacaagaccaccccccctgtgctggacagcgacggctcattcttcctgtacagcaagctgaccgtggacaagtcccggtggcagcagggcaacgtgttcagctgcagcgtgatgcacgaggccctgcacaaccactacacccagaagtccctgagcctgagccctggcaag

SEQ ID NO: 841 - DNA, 642 bp, synthetic construct
  misc_feature 1..642: note = antibody sequence
  source 1..642: mol_type = other DNA
gagattgtgctgacacagtctcccgccacactgtctcttagccctggcgaaagagccacactgagctgtagagccagccagagcatcggcgattacctgcactggtatcagcagaagcctggacaggcccctcggctgctgatctactatgccagccagtccatcagcggcatccccgccagattttctggcagcggctctggcaccgatttcaccctgaccataagcagcctggaacctgaggacttcgccgtgtactactgccagcagggctacagcttcccctacacatttggcggaggcaccaagctggaaatcaaacgaaccgtggccgctcccagcgtgttcatcttcccacctagcgacgagcagctgaagtccggcacagcctctgtcgtgtgcctgctgaacaacttctacccccgcgaggccaaggtgcagtggaaggtggacaatgccctgcagagcggcaacagccaggaaagcgtgaccgagcaggacagcaaggactccacctacagcctgagcagcaccctgaccctgagcaaggccgactacgagaagcacaaggtgtacgcctgcgaagtgacccaccagggcctgtctagccccgtgaccaagagcttcaaccggggcgagtgt

SEQ ID NO: 842 - protein, 117 aa, synthetic construct
  REGION 1..117: note = antibody sequence
EVQLVESGGGLLQLGGSLRLSCAASGFTFSSYYMSWVRQAPGKGLEWVSTIIKSGGYAYYPDSVKDRFTISRDNSKNTLNLQMNSLRAEDTAVYYCVKGGQGAMDYWGQGTTVTVSS

SEQ ID NO: 843 - protein, 5 aa, synthetic construct
  REGION 1..5: note = antibody sequence
SYYMS

SEQ ID NO: 844 - protein, 17 aa, synthetic construct
  REGION 1..17: note = antibody sequence
TIIKSGGYAYYPDSVKD

SEQ ID NO: 845 - protein, 8 aa, synthetic construct
  REGION 1..8: note = antibody sequence
GGQGAMDY

SEQ ID NO: 846 - protein, 107 aa, synthetic construct
  REGION 1..107: note = antibody sequence
EIVLTQSPATLSLSPGERATLSCRASQSIGDYLHWYQQKPGQAPRLLIKYASQSISGIPARFSGSGSGTDFTLTISSLEPEDFAVYYCQQGYSFPYTFGGGTKLEIK

SEQ ID NO: 847 - protein, 11 aa, synthetic construct
  REGION 1..11: note = antibody sequence
RASQSIGDYLH

SEQ ID NO: 848 - protein, 7 aa, synthetic construct
  REGION 1..7: note = antibody sequence
YASQSIS

SEQ ID NO: 849 - protein, 9 aa, synthetic construct
  REGION 1..9: note = antibody sequence
QQGYSFPYT

SEQ ID NO: 850 - DNA, 351 bp, synthetic construct
  misc_feature 1..351: note = antibody sequence
  source 1..351: mol_type = other DNA
gaagtgcagctggtggaatctggcggaggactgctgcagcttggcggatctctgagactgtcttgtgccgccagcggcttcaccttcagcagctactacatgagctgggtccgacaggcccctggcaaaggacttgaatgggtgtccaccatcatcaagagcggcggctacgcctactatcccgacagcgtgaaggaccggttcaccatctccagagacaacagcaagaacaccctgaacctgcagatgaacagcctgagagccgaggacaccgccgtgtactactgtgtgaaaggtggacagggcgccatggactattggggccagggaacaacagtgaccgtgtcctca

SEQ ID NO: 851 - DNA, 321 bp, synthetic construct
  misc_feature 1..321: note = antibody sequence
  source 1..321: mol_type = other DNA
gagattgtgctgacacagtctcccgccacactgtctcttagccctggcgaaagagccacactgagctgtagagccagccagagcatcggcgattacctgcactggtatcagcagaagcctggacaggcccctcggctgctgattaagtacgccagccagtccatcagcggcatccctgccagattttctggcagcggctctggcaccgatttcaccctgaccataagcagcctggaacctgaggacttcgccgtgtactactgccagcagggctacagcttcccctacacatttggcggaggcaccaagctggaaatcaaa

SEQ ID NO: 852 - protein, 447 aa, synthetic construct
  REGION 1..447: note = antibody sequence
EVQLVESGGGLLQLGGSLRLSCAASGFTFSSYYMSWVRQAPGKGLEWVSTIIKSGGYAYYPDSVKDRFTISRDNSKNTLNLQMNSLRAEDTAVYYCVKGGQGAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 853 - protein, 214 aa, synthetic construct
  REGION 1..214: note = antibody sequence
EIVLTQSPATLSLSPGERATLSCRASQSIGDYLHWYQQKPGQAPRLLIKYASQSISGIPARFSGSGSGTDFTLTISSLEPEDFAVYYCQQGYSFPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 854 - DNA, 1341 bp, synthetic construct
  misc_feature 1..1341: note = antibody sequence
  source 1..1341: mol_type = other DNA
gaagtgcagctggtggaatctggcggaggactgctgcagcttggcggatctctgagactgtcttgtgccgccagcggcttcaccttcagcagctactacatgagctgggtccgacaggcccctggcaaaggacttgaatgggtgtccaccatcatcaagagcggcggctacgcctactatcccgacagcgtgaaggaccggttcaccatctccagagacaacagcaagaacaccctgaacctgcagatgaacagcctgagagccgaggacaccgccgtgtactactgtgtgaaaggtggacagggcgccatggactattggggccagggaacaacagtgaccgtgtcctcagccagcaccaagggccccagcgtgttccctctggcccctagcagcaagagcacatctggcggaacagccgccctgggctgcctcgtgaaggactactttcccgagcccgtgaccgtgtcctggaactctggcgctctgacaagcggcgtgcacacctttccagccgtgctgcagagcagcggcctgtactctctgagcagcgtcgtgacagtgcccagcagctctctgggcacccagacctacatctgcaacgtgaaccacaagcccagcaacaccaaggtggacaagaaggtggaacccaagagctgcgacaagacccacacctgtcccccttgtcctgcccccgaactgctgggaggcccttccgtgttcctgttccccccaaagcccaaggacaccctgatgatcagccggacccccgaagtgacctgcgtggtggtggatgtgtcccacgaggaccctgaagtgaagttcaattggtacgtggacggcgtggaagtgcacaacgccaagaccaagcctagagaggaacagtacaacagcacctaccgggtggtgtccgtgctgacagtgctgcaccaggactggctgaacggcaaagagtacaagtgcaaggtgtccaacaaggccctgcctgcccccatcgagaaaaccatcagcaaggccaagggccagccccgcgaaccccaggtgtacacactgcccccaagcagggacgagctgaccaagaaccaggtgtccctgacctgtctcgtgaaaggcttctacccctccgatatcgccgtggaatgggagagcaacggccagcccgagaacaactacaagaccaccccccctgtgctggacagcgacggctcattcttcctgtacagcaagctgaccgtggacaagtcccggtggcagcagggcaacgtgttcagctgcagcgtgatgcacgaggccctgcacaaccactacacccagaagtccctgagcctgagccctggcaag

SEQ ID NO: 855 - DNA, 642 bp, synthetic construct
  misc_feature 1..642: note = antibody sequence
  source 1..642: mol_type = other DNA
gagattgtgctgacacagtctcccgccacactgtctcttagccctggcgaaagagccacactgagctgtagagccagccagagcatcggcgattacctgcactggtatcagcagaagcctggacaggcccctcggctgctgattaagtacgccagccagtccatcagcggcatccctgccagattttctggcagcggctctggcaccgatttcaccctgaccataagcagcctggaacctgaggacttcgccgtgtactactgccagcagggctacagcttcccctacacatttggcggaggcaccaagctggaaatcaaacgaaccgtggccgctcccagcgtgttcatcttcccacctagcgacgagcagctgaagtccggcacagcctctgtcgtgtgcctgctgaacaacttctacccccgcgaggccaaggtgcagtggaaggtggacaatgccctgcagagcggcaacagccaggaaagcgtgaccgagcaggacagcaaggactccacctacagcctgagcagcaccctgaccctgagcaaggccgactacgagaagcacaaggtgtacgcctgcgaagtgacccaccagggcctgtctagccccgtgaccaagagcttcaaccggggcgagtgt

SEQ ID NO: 856 - protein, 122 aa, synthetic construct
  REGION 1..122: note = antibody sequence
EVQLLESGGGLVQPGGSLRLSCAASGFTFRSYAVHWVRQAPGKGLEWVSSTEGSGVGTSYTDSVKGRFTISRDNSKNTLYLQMNSLRAEDTAVYYCARMLGGGNPLDYLDYWGQGTLVTVSS

SEQ ID NO: 857 - protein, 5 aa, synthetic construct
  REGION 1..5: note = antibody sequence
SYAVH

SEQ ID NO: 858 - protein, 17 aa, synthetic construct
  REGION 1..17: note = antibody sequence
STEGSGVGTSYTDSVKG

SEQ ID NO: 859 - protein, 13 aa, synthetic construct
  REGION 1..13: note = antibody sequence
MLGGGNPLDYLDY

SEQ ID NO: 860 - protein, 111 aa, synthetic construct
  REGION 1..111: note = antibody sequence
QSVLTQPPSASGTPGQRVTISCSGSSSNLGEGYDVHWYQQLPGKAPKLLIYYSDFRPSGVSDRFSGSKSGTSASLAISGLQSEDEADYYCAAWDDSLSSQVFGGGTQVTVL

SEQ ID NO: 861 - protein, 14 aa, synthetic construct
  REGION 1..14: note = antibody sequence
SGSSSNLGEGYDVH

SEQ ID NO: 862 - protein, 7 aa, synthetic construct
  REGION 1..7: note = antibody sequence
YSDFRPS

SEQ ID NO: 863 - protein, 11 aa, synthetic construct
  REGION 1..11: note = antibody sequence
AAWDDSLSSQV

SEQ ID NO: 864 - DNA, 366 bp, synthetic construct
  misc_feature 1..366: note = antibody sequence
  source 1..366: mol_type = other DNA
gaagttcagctgctggaatctggcggcggactggttcaacctggcggatctctgagactgagctgtgccgccagcggcttcacctttagaagctatgccgtgcactgggtccgacaggcccctggaaaaggactggaatgggtgtccagcaccgaaggctctggcgtgggcacaagctacaccgattctgtgaagggcagattcaccatcagccgggacaacagcaagaacaccctgtacctgcagatgaacagcctgagagccgaggacaccgccgtgtactactgtgccagaatgctcggcggaggcaaccctctggactacctggattattggggccagggcaccctggtcacagtctcttca

SEQ ID NO: 865 - DNA, 333 bp, synthetic construct
  misc_feature 1..333: note = antibody sequence
  source 1..333: mol_type = other DNA
cagtctgttctgacacagcctcctagcgcctctggcacacctggacagagagtgaccatcagctgtagcggcagcagctccaatctcggcgagggctatgacgtgcactggtatcagcagctgcctggcaaggcccctaaactgctgatctactacagcgacttcagacccagcggcgtgtccgatagattcagcggctctaagagcggcacatctgccagcctggccatctctggactgcagagcgaagatgaggccgactactattgcgccgcctgggatgatagcctgagcagccaagtttttggcggcggaacccaagtgaccgtgcta

SEQ ID NO: 866 - protein, 452 aa, synthetic construct
  REGION 1..452: note = antibody sequence
EVQLLESGGGLVQPGGSLRLSCAASGFTFRSYAVHWVRQAPGKGLEWVSSTEGSGVGTSYTDSVKGRFTISRDNSKNTLYLQMNSLRAEDTAVYYCARMLGGGNPLDYLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 867 - protein, 217 aa, synthetic construct
  REGION 1..217: note = antibody sequence
QSVLTQPPSASGTPGQRVTISCSGSSSNLGEGYDVHWYQQLPGKAPKLLIYYSDFRPSGVSDRFSGSKSGTSASLAISGLQSEDEADYYCAAWDDSLSSQVFGGGTQVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 868 - DNA, 1356 bp, synthetic construct
  misc_feature 1..1356: note = antibody sequence
  source 1..1356: mol_type = other DNA
gaagttcagctgctggaatctggcggcggactggttcaacctggcggatctctgagactgagctgtgccgccagcggcttcacctttagaagctatgccgtgcactgggtccgacaggcccctggaaaaggactggaatgggtgtccagcaccgaaggctctggcgtgggcacaagctacaccgattctgtgaagggcagattcaccatcagccgggacaacagcaagaacaccctgtacctgcagatgaacagcctgagagccgaggacaccgccgtgtactactgtgccagaatgctcggcggaggcaaccctctggactacctggattattggggccagggcaccctggtcacagtctcttcagccagcaccaagggccccagcgtgttccctctggcccctagcagcaagagcacatctggcggaacagccgccctgggctgcctcgtgaaggactactttcccgagcccgtgaccgtgtcctggaactctggcgctctgacaagcggcgtgcacacctttccagccgtgctgcagagcagcggcctgtactctctgagcagcgtcgtgacagtgcccagcagctctctgggcacccagacctacatctgcaacgtgaaccacaagcccagcaacaccaaggtggacaagaaggtggaacccaagagctgcgacaagacccacacctgtcccccttgtcctgcccccgaactgctgggaggcccttccgtgttcctgttccccccaaagcccaaggacaccctgatgatcagccggacccccgaagtgacctgcgtggtggtggatgtgtcccacgaggaccctgaagtgaagttcaattggtacgtggacggcgtggaagtgcacaacgccaagaccaagcctagagaggaacagtacaacagcacctaccgggtggtgtccgtgctgacagtgctgcaccaggactggctgaacggcaaagagtacaagtgcaaggtgtccaacaaggccctgcctgcccccatcgagaaaaccatcagcaaggccaagggccagccccgcgaaccccaggtgtacacactgcccccaagcagggacgagctgaccaagaaccaggtgtccctgacctgtctcgtgaaaggcttctacccctccgatatcgccgtggaatgggagagcaacggccagcccgagaacaactacaagaccaccccccctgtgctggacagcgacggctcattcttcctgtacagcaagctgaccgtggacaagtcccggtggcagcagggcaacgtgttcagctgcagcgtgatgcacgaggccctgcacaaccactacacccagaagtccctgagcctgagccctggcaag

SEQ ID NO: 869 - DNA, 651 bp, synthetic construct
  misc_feature 1..651: note = antibody sequence
  source 1..651: mol_type = other DNA
cagtctgttctgacacagcctcctagcgcctctggcacacctggacagagagtgaccatcagctgtagcggcagcagctccaatctcggcgagggctatgacgtgcactggtatcagcagctgcctggcaaggcccctaaactgctgatctactacagcgacttcagacccagcggcgtgtccgatagattcagcggctctaagagcggcacatctgccagcctggccatctctggactgcagagcgaagatgaggccgactactattgcgccgcctgggatgatagcctgagcagccaagtttttggcggcggaacccaagtgaccgtgctaggccagcctaaagccgcccctagcgtgaccctgttccctccaagcagcgaggaactgcaggccaacaaggccaccctcgtgtgcctgatcagcgacttctatcctggcgccgtgaccgtggcctggaaggccgatagctctcctgtgaaggccggcgtggaaaccaccacccctagcaagcagagcaacaacaaatacgccgccagcagctacctgagcctgacccccgagcagtggaagtcccacagatcctacagctgccaagtgacccacgagggcagcaccgtggaaaagacagtggcccctaccgagtgcagc

SEQ ID NO: 870 - protein, 122 aa, synthetic construct; identical to SEQ ID NO: 856
  REGION 1..122: note = antibody sequence

SEQ ID NO: 871 - protein, 5 aa, synthetic construct
  REGION 1..5: note = antibody sequence
SYAVH

SEQ ID NO: 872 - protein, 17 aa, synthetic construct
  REGION 1..17: note = antibody sequence
STEGSGVGTSYTDSVKG

SEQ ID NO: 873 - protein, 13 aa, synthetic construct
  REGION 1..13: note = antibody sequence
MLGGGNPLDYLDY

SEQ ID NO: 874 - protein, 111 aa, synthetic construct; identical to SEQ ID NO: 860
  REGION 1..111: note = antibody sequence

SEQ ID NO: 875 - protein, 14 aa, synthetic construct
  REGION 1..14: note = antibody sequence
SGSSSNLGEGYDVH

SEQ ID NO: 876 - protein, 7 aa, synthetic construct
  REGION 1..7: note = antibody sequence
YSDFRPS

SEQ ID NO: 877 - protein, 11 aa, synthetic construct
  REGION 1..11: note = antibody sequence
AAWDDSLSSQV

SEQ ID NO: 878 - DNA, 366 bp, synthetic construct; identical to SEQ ID NO: 864
  misc_feature 1..366: note = antibody sequence
  source 1..366: mol_type = other DNA

SEQ ID NO: 879 - DNA, 333 bp, synthetic construct; identical to SEQ ID NO: 865
  misc_feature 1..333: note = antibody sequence
  source 1..333: mol_type = other DNA

SEQ ID NO: 880 - protein, 251 aa, synthetic construct
  REGION 1..251: note = antibody sequence
EVQLLESGGGLVQPGGSLRLSCAASGFTFRSYAVHWVRQAPGKGLEWVSSTEGSGVGTSYTDSVKGRFTISRDNSKNTLYLQMNSLRAEDTAVYYCARMLGGGNPLDYLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAAGSEQKLISEEDLSGSAAAHHHHHH

SEQ ID NO: 881 - protein, 217 aa, synthetic construct; identical to SEQ ID NO: 867
  REGION 1..217: note = antibody sequence

SEQ ID NO: 882 - DNA, 753 bp, synthetic construct
  misc_feature 1..753: note = antibody sequence
  source 1..753: mol_type = other DNA
gaagttcagctgctggaatctggcggcggactggttcaacctggcggatctctgagactgagctgtgccgccagcggcttcacctttagaagctatgccgtgcactgggtccgacaggcccctggaaaaggactggaatgggtgtccagcaccgaaggctctggcgtgggcacaagctacaccgattctgtgaagggcagattcaccatcagccgggacaacagcaagaacaccctgtacctgcagatgaacagcctgagagccgaggacaccgccgtgtactactgtgccagaatgctcggcggaggcaaccctctggactacctggattattggggccagggcaccctggtcacagtctcttcagcctccaccaagggcccatcggtgttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgcagcgggttctgaacaaaaactcatctcagaagaggatctgtctggatcagcggccgcccatcatcatcatcatcat

SEQ ID NO: 883 - DNA, 651 bp, synthetic construct; identical to SEQ ID NO: 869
  misc_feature 1..651: note = antibody sequence
  source 1..651: mol_type = other DNA

SEQ ID NO: 884 - protein, 563 aa, Oryctolagus cuniculus
NYQNGKNNVPRLKLSYKEMLESNNVITFNGLANSSSYHTFLLDEERSRLYVGAKDHIFSFNLVNIKDFQKIAWPVSYTRRDECKWAGKDILRECANFIKVLKVYNQTHLYACGTGAFHPICTYVGIGHHPEDNIFKLEDSHFENGRGKSPYDPKLLTASLLIDGELYSGTAADFMGRDFAIFRTLGQHHPIRTEQHDSRWLNDPRFISAHLIPESDNPEDDKVYFFFRENAIDGEHSGKATHARIGQICKNDFGGHRSLVNKWTTFLKARLICSVPGPNGIDTHFDELQDVFLMNSKDPKNPIVYGVFTTSSNIFRGSAVCMYSMSDVRRVFLGPYAHRDGPNYQWVPFQGRVPYPRPGTCPSKTFGGFESTKDLPDDVITFARSHPAMYNPVFPINNRPIMVKTDVNYQFTQIVVDRVDAEDGQYDVMFIGTDVGTVLKVVSIPKETWHDLEEVLLEEMTVFREPTTISAMELSTKQQQLYVGSAAGVAQLPLHRCDIYGKACAECCLARDPYCAWDGSSCSRYFPTAKRRTRRQDIRNGDPLTHCSDGGIEGRMDHHHHHH

SEQ ID NO: 885 - DNA, 1689 bp, Oryctolagus cuniculus
  source 1..1689: mol_type = other DNA
aactatcagaacggcaagaacaacgtgccccggctgaagctgagctacaaagagatgctggaaagcaacaacgtgatcaccttcaacggcctggccaacagcagcagctaccacacctttctgctggacgaggaacggtccagactgtacgtgggagccaaggaccacatcttcagcttcaacctggtcaacatcaaggacttccagaaaatcgcctggcctgtgtcctacaccagacgggatgagtgtaaatgggccggcaaggacatcctgagagagtgcgccaacttcatcaaggtgctgaaggtgtacaatcagacccacctgtacgcctgtggcaccggcgcttttcaccctatctgtacctatgtcggcatcggccaccatcctgaggacaatatcttcaagctcgaggacagccacttcgagaacggcagaggcaagagcccctacgatcccaaactgctgacagcctctctgctgatcgacggcgagctgtattctggcacagccgccgatttcatgggcagagacttcgccatcttcagaaccctgggccagcatcaccccatcagaaccgagcagcacgacagcagatggctgaacgaccccagattcatcagcgcccatctgatccccgagagcgacaaccccgaggacgacaaggtgtacttcttcttccgggaaaacgccatcgacggggagcactctggaaaagccacacacgccagaatcggccagatctgcaagaacgacttcggcggccacagatccctcgtgaacaagtggaccaccttcctgaaggcccggctgatctgttctgtgcccggacctaatggcatcgatacccacttcgacgagctgcaggacgtgttcctgatgaacagcaaggaccccaagaatcccatcgtgtacggcgtgttcaccaccagcagcaacatctttagaggcagcgccgtgtgcatgtacagcatgtccgatgtgcggagagtgtttctgggcccctacgctcacagagatggccccaattatcagtgggtgccattccagggcagagtgccctatcctagacctggcacctgtcctagcaagacctttggcggcttcgagagcaccaaggacctgcctgacgatgtgattaccttcgccagatctcaccccgccatgtacaaccctgtgttccccatcaacaacaggcccatcatggtcaagaccgacgtgaactaccagttcacccagatcgtggtggacagagtggatgccgaggacggccagtacgacgtgatgttcatcggcaccgatgtgggcaccgtgctgaaagtggtgtctatccccaaagagacatggcacgacctggaagaggtgctgctggaagagatgaccgtgttcagagagcccaccaccatctccgccatggaactgagcacaaaacagcaacagctgtatgtgggctccgccgctggtgttgctcaactgcctctgcacagatgcgacatctacggcaaagcctgcgccgagtgttgcctggccagagatccttactgtgcctgggatggcagcagctgcagcagatactttcccaccgccaagcggagaaccagacggcaggatatcagaaacggcgaccctctgacacactgcagcgacggtggcatcgagggccgcatggatcatcatcatcaccatcat

(19) United States
(12) Patent Application Publication
(10) Pub. No.: US 2024/0254222 A1
(43) Pub. Date: Aug. 1, 2024

(54) Novel Anti-Sema3a Antibodies And Uses Thereof

(71) Applicant: BAYER AKTIENGESELLSCHAFT, Leverkusen (DE)

(72) Inventors: Dorian SCHÖNFELD, Köln (DE); Karoline DRÖBNER, Velbert (DE); Ernst WEBER, Langenfeld (DE); Katharina FILARSKY, Düsseldorf (DE); Philipp ELLINGER, Solingen (DE); Fionnuala Mary MCALEESE ESER, Lagenfeld (DE); Ingo FLAMME, Reichshof (DE); Winfried WUNDERLICH, Bovenden (DE); Antje SCHMIDT, Göttingen (DE); Yalda SEDAGHAT, Hamburg (DE); Kenneth YOUNG, Hamburg (DE)

(21) Appl. No.: 18/399,934

(22) Filed: Dec. 29, 2023

Related U.S. Application Data
(63) Continuation of application No. 17/706,543, filed on Mar. 28, 2022.

(30) Foreign Application Priority Data
Mar. 30, 2021 (EP) 21165960.2

(51) Int. Cl. C07K 16/28; A61K 39/00; A61K 39/395; A61P 9/10; A61P 13/12; C07K 16/18

(52) U.S. Cl. CPC C07K 16/2803; A61K 39/3955; A61P 9/10; A61P 13/12; C07K 16/18; A61K 2039/505; C07K 2317/33; C07K 2317/565; C07K 2317/76; C07K 2317/92

(57) ABSTRACT

The present disclosure relates to an isolated antibody or antigen-binding fragment thereof that binds to human Sema3A. The isolated antibody or antigen-binding fragment according to the present disclosure i) binds to human Sema3A of the sequence of SEQ ID NO: 600 with a dissociation constant (KD)≤50 nM, ≤20 nM, ≤10 nM, ≤1 nM, or ≤0.1 nM; ii) cross-reacts with mouse, cynomolgus, rat, pig and/or dog Sema3A, particularly wherein said isolated antibody or antigen-binding fragment thereof binds to mouse, cynomolgus, rat, pig and/or dog Sema3A with a dissociation constant (KD)≤50 nM, ≤20 nM, ≤10 nM, ≤1 nM, or ≤0.1 nM; iii) binds to human Sema3A of the sequence of SEQ ID NO: 600 with a binding activity as measured by surface plasmon resonance (SPR) of ≥60%, ≥70%, ≥80%, or ≥90%; iv) inhibits the activity of human Sema3A of the sequence of SEQ ID NO: 600 in an in vitro mesangial cell migration assay with an EC50 of ≤10 nM, ≤5 nM, ≤2.5 nM, or ≤1 nM; v) inhibits the activity of human Sema3A of the sequence of SEQ ID NO: 600 in an in vitro growth cone collapse assay with an EC50 of ≤50 nM, ≤25 nM, ≤10 nM, or ≤5 nM; vi) inhibits the activity of human Sema3A of the sequence of SEQ ID NO: 600 in an in vitro HUVEC repulsion assay with an EC50 of ≤1 nM, or ≤0.3 nM, ≤0.1 nM, ≤0.07 nM, ≤0.06 nM and/or vii) exhibits an increased potency against cellular Sema3A, of the sequence of SEQ ID NO: 600, induced HUVEC repulsion.

### CROSS-REFERENCE TO RELATED APPLICATION

This application is a continuation of U.S. patent application Ser. No. 17/706,543, filed Mar. 28, 2022, which claims the benefit of, and priority to, European Patent Application Serial No. 21165960.2, filed Mar. 30, 2021. The entire disclosure of each of the above applications is hereby incorporated by reference.

### SEQUENCE LISTING

This application contains references to amino acid sequences and/or nucleic acid sequences which have been submitted concurrently herewith as the sequence listing .xml file entitled “000034uscoa_SequenceListing.xml”, file size 1,040,371 bytes, created on Dec. 28, 2023. The aforementioned sequence listing is hereby incorporated by reference in its entirety pursuant to 37 C.F.R. § 1.52(e)(5).

### FIELD

The present disclosure provides isolated antibodies or antigen-binding fragments thereof that bind to human semaphorin 3A (Sema3A). The isolated antibody or antigen-binding fragments according to the present disclosure i) bind to human Sema3A of the sequence of SEQ ID NO: 600 with a dissociation constant (KD)≤50 nM, ≤20 nM, ≤10 nM, ≤1 nM, or ≤0.1 nM; ii) cross-react with mouse, cynomolgus, rat, pig and/or dog Sema3A, particularly wherein said isolated antibodies or antigen-binding fragments thereof binds to mouse, cynomolgus, rat, pig and/or dog Sema3A with a dissociation constant (KD)≤50 nM, ≤20 nM, ≤10 nM, ≤1 nM, or ≤0.1 nM; iii) bind to human Sema3A of the sequence of SEQ ID NO: 600 with a binding activity as measured by surface plasmon resonance (SPR) of ≥60%, ≥70%, ≥80%, or ≥90%; iv) inhibit the activity of human Sema3A of the sequence of SEQ ID NO: 600 in an in vitro mesangial cell migration assay with an EC50 of ≤10 nM, ≤5 nM, ≤2.5 nM, or ≤1 nM; v) inhibit the activity of human Sema3A of the sequence of SEQ ID NO: 600 in an in vitro growth cone collapse assay with an EC50 of ≤50 nM, ≤25 nM, ≤10 nM, or ≤5 nM; and/or vi) inhibit the activity of human Sema3A of the sequence of SEQ ID NO: 600 in an in vitro HUVEC repulsion assay with an EC50 of ≤1 nM, or ≤0.3 nM, ≤0.1 nM, ≤0.07 nM, ≤0.06 nM and/or vii) exhibiting an increased potency against cellular Sema3A, of the sequence of SEQ ID NO: 600, induced HUVEC repulsion. The present disclosure further provides isolated nucleic acid sequences encoding said antibodies or antigen-binding fragments thereof and vectors comprising same, isolated cells expressing said antibodies or antigen-binding fragments thereof, methods of producing said antibodies or antigen-binding fragments thereof and pharmaceutical compositions and kits comprising said antibodies or antigen-binding fragments thereof.

Antibodies according to the present disclosure can be used in the treatment of diseases associated with increased Sema3A levels or activity such as Alport syndrome, acute kidney injury (AKI) primary focal segmental glomerular sclerosis (FSGS), or chronic kidney disease (CKD).

### BACKGROUND

Semaphorin 3A (Sema3A) is a secreted dimeric protein that acts as guidance protein. It forms a ternary complex with neuropilin-1 and different plexins which leads to the activation of different signaling pathways. It is a key regulator of cell migration, adhesion, cytoskeletal stabilization and apoptosis. Sema3A is expressed in podocyte in adult kidneys where it is induced after injury.

Excess of Sema3A interferes with the glomerular filtration barrier inducing ultrastructural changes of the filtration barrier leading to podocyte foot process effacement and albuminuria. Sema3A is also highly induced after AKI and exacerbates the injury by promoting tubular inflammation, tubular epithelial cell apoptosis and ultrastructural abnormalities of the filtration barrier. Genetic deficiency or pharmacological inhibition of Sema3A in rodents results in reduced renal damage in different animal models of kidney diseases.

Furthermore, Sema3A is expressed in retinal neurons and endothelium. It has been shown to increase vascular permeability, to promote retinal inflammation and cellular senescence and to inhibit retinal vascular regeneration in rodent models. Sema3A also plays a role in CNS disorders. Sema3A inhibition results in enhanced regeneration and/or preservation of injured axons, decreased apoptotic cell numbers and enhancement of angiogenesis, resulting in considerably better functional recovery.

WO 20141/23186 discloses an avian-mouse chimeric antibody (clone No. 4-2 strain-derived) and two humanized IgG1 variants thereof and suggests their suitability in the treatment of Alzheimer's disease.

WO 2017/074013 discloses anti-Sema3A IgG antibodies A08, C10 and F11 and suggests their suitability in the treatment of cancer.

Currently, no therapeutic option to inhibit Sema3A interaction with its receptors is available to treat patients with e.g. proteinuric kidney disease like Alport syndrome and it is presumed that monoclonal therapeutic Sema3A antibodies could be optimally suited for this. Thus, there exists a great need for novel therapeutic Sema3A antibodies useful for the treatment of diseases that are associated with elevated Sema3A levels or activity such as Alport syndrome, acute kidney injury (AKI) primary focal segmental glomerular sclerosis (FSGS), or chronic kidney disease (CKD) that has not been met so far.

### Objects

In view of the prior art, it is an object of the present disclosure to provide novel therapeutic Sema3A antibodies that overcome the shortcomings of Sema3A antibodies of the prior art. In particular it is an object of the present disclosure to provide novel Sema3A antibodies that are high affinity binders of human Sema3A that efficiently block Sema3A activity. Desirable Sema3A antibodies are cross-reactive to Sema3A of multiple species in order to allow for preclinical experiments. They are non-immunogenic in human therapy, i.e. they are human or humanized antibodies. Desirable Sema3A antibodies are selective to Sema3A; they do not bind to off-targets and in particular do not cross-react with other semaphorin protein family members.

Such novel Sema3A antibodies would offer major advances in the treatment of diseases associated with elevated Sema3A levels or activity such as Alport syndrome, acute kidney injury (AKI) primary focal segmental glomerular sclerosis (FSGS), or chronic kidney disease (CKD).

### SUMMARY

The above-mentioned object and other objects are achieved by the teaching of the present disclosure. The present disclosure is based on the discovery of novel antibodies that have a specific affinity for Sema3A and can deliver a therapeutic benefit to a subject.

Thus, in a first aspect, the present disclosure relates to an isolated antibody or antigen-binding fragment thereof that that binds to human Sema3A, wherein said isolated antibody or antigen-binding fragment thereof i) binds to human Sema3A of the sequence of SEQ ID NO: 600 with a dissociation constant (KD)≤50 nM, ≤20 nM, ≤10 nM, ≤1 nM, or ≤0.1 nM; ii) cross-reacts with mouse, cynomolgus, rat, pig and/or dog Sema3A, particularly wherein said isolated antibody or antigen-binding fragment thereof binds to mouse, cynomolgus, rat, pig and/or dog Sema3A with a dissociation constant (KD)≤50 nM, ≤20 nM, ≤10 nM, ≤1 nM, or ≤0.1 nM; iii) binds to human Sema3A of the sequence of SEQ ID NO: 600 with a binding activity as measured by surface plasmon resonance (SPR) of ≥60%, ≥70%, ≥80%, or ≥90%; iv) inhibits the activity of human Sema3A of the sequence of SEQ ID NO: 600 in an in vitro mesangial cell migration assay with an EC50 of ≤10 nM, ≤5 nM, ≤2.5 nM, or ≤1 nM; v) inhibits the activity of human Sema3A of the sequence of SEQ ID NO: 600 in an in vitro growth cone collapse assay with an EC50 of ≤50 nM, ≤25 nM, ≤10 nM, or ≤5 nM; vi) inhibits the activity of human Sema3A of the sequence of SEQ ID NO: 600 in an in vitro HUVEC repulsion assay with an EC50 of ≤1 nM, or ≤0.3 nM, ≤0.1 nM, ≤0.07 nM, ≤0.06 nM and/or vii) exhibits an increased potency against cellular Sema3A, of the sequence of SEQ ID NO: 600, induced HUVEC repulsion.

The isolated antibody or antigen-binding fragment according to the present disclosure binds with high affinity to human Sema3A and inhibits its function. Thus, the isolated antibody or antigen-binding fragment according to the present disclosure may be used in the treatment of diseases associated with increased Sema3A levels or activity such as i) renal diseases, in particular acute and chronic kidney diseases, diabetic kidney diseases, Alport syndrome, acute and chronic renal failure, polycystic kidney disease (PCKD) and syndrome of inadequate ADH secretion (SIADH); ii) sequelae of renal insufficiency, in particular pulmonary edema, heart failure, uremia, anemia, electrolyte disturbances such as hyperkaliemia and hyponatremia and disturbances in bone and carbohydrate metabolism; iii) vascular hyperpermeability, diabetic retinopathy, deterioration of the blood retinal barrier, macular edema, particularly age related macular edema, non-proliferative age-related macular edema and non-proliferative diabetic macular edema; iv) diseases of the central or peripheral nervous system in particular neuropathic pain, spinal cord injury, multiple sclerosis, traumatic brain injury, brain edema and neurodegenerative diseases, particularly Alzheimer's disease, Parkinson's disease, Huntington's disease, amyotrophic lateral sclerosis, progressive supranuclear paralysis, black substance degeneration, Shy-Drager syndrome, olivopontocerebellar atrophy and spinocerebellar degeneration; v) cancer, in particular intestinal cancer, colorectal cancer, lung cancer, breast cancer, brain cancer, melanoma, renal cell cancer, leukemia, lymphoma, T-cell lymphoma, stomach cancer, pancreatic cancer, cervical cancer, endometrial cancer, ovarian cancer, esophageal cancer, liver cancer, squamous cell carcinoma of the head and neck, skin cancer, urinary tract cancer, prostate cancer, choriocarcinoma, pharyngeal cancer and larynx cancer.

The isolated antibody or antigen-binding fragment according to the present disclosure may further be used in the diagnosis of Sema3A-related disorders.

In a further aspect, the present disclosure relates to an isolated nucleic acid sequence that encodes the antibody or antigen-binding fragment according to the present disclosure.

In a further aspect, the present disclosure relates to a vector comprising a nucleic acid sequence according to the present disclosure.

In a further aspect, the present disclosure relates to an isolated cell expressing the antibody or antigen-binding fragment according to the present disclosure and/or comprising the nucleic acid according to the present disclosure or the vector according to the present disclosure.

In a further aspect, the present disclosure relates to a method of producing the isolated antibody or antigen-binding fragment according to the present disclosure comprising culturing of the cell according to the present disclosure and optionally purification of the antibody or antigen-binding fragment thereof.

In a further aspect, the present disclosure relates to a pharmaceutical composition comprising the isolated antibody or antigen-binding fragment according to the present disclosure or the antibody conjugate according to the present disclosure.

In a further aspect, the present disclosure relates to a kit comprising the isolated antibody or antigen-binding fragment according to the present disclosure or the conjugate according to the present disclosure and instructions for use.

### DETAILED DESCRIPTION

The present disclosure may be understood more readily by reference to the following detailed description of the disclosure and the examples included therein.

### Definitions

Unless defined otherwise, all technical and scientific terms used herein have the meaning commonly understood by one of ordinary skill in the art to which this disclosure belongs. The following references, however, can provide one of skill in the art to which this disclosure pertains with a general definition of many of the terms used in this disclosure, and can be referenced and used so long as such definitions are consistent with the meaning commonly understood in the art. Such references include, but are not limited to, Singleton et al., Dictionary of Microbiology and Molecular Biology (2nd ed. 1994); The Cambridge Dictionary of Science and Technology (Walker ed., 1988); Hale & Marham, The Harper Collins Dictionary of Biology (1991); Lackie et al., The Dictionary of Cell & Molecular Biology (3d ed. 1999); and Cellular and Molecular Immunology, Eds. Abbas, Lichtman and Pober, 2nd Edition, W.B. Saunders Company. Any additional technical resource available to the person of ordinary skill in the art providing definitions of terms used herein having the meaning commonly understood in the art can be consulted. For the purposes of the present disclosure, the following terms are further defined. Additional terms are defined elsewhere in the description. As used herein and in the appended claims, the singular forms “a,” and “the” include plural reference unless the context clearly dictates otherwise. Thus, for example, reference to “a gene” is a reference to one or more genes and includes equivalents thereof known to those skilled in the art, and so forth.

In the context of the present disclosure, the term “comprises” or “comprising” means “including, but not limited to”. The term is intended to be open-ended, to specify the presence of any stated features, elements, integers, steps or components, but not to preclude the presence or addition of one or more other features, elements, integers, steps, components or groups thereof. The term “comprising” thus includes the more restrictive terms “consisting of” and “essentially consisting of”. In one embodiment the term “comprising” as used throughout the application and in particular within the claims may be replaced by the term “consisting of”.

In this context, the term “about” or “approximately” means within 80% to 120%, alternatively within 90% to 110%, including within 95% to 105% of a given value or range.

The terms “polypeptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. Unless otherwise indicated, a particular polypeptide sequence also implicitly encompasses conservatively modified variants thereof.

As used herein “Sema3A” designates “semaphorin 3A”, also known as “HH16”, “SemD”, “COLL1”, “SEMA1”, “SEMAD”, “SEMAL”, “coll-1”, “Hsema-I”, “SEMAIII”, “Hsema-III”, “collapsin 1”, “semaphorin D”, “semaphorin III”, and “semaphorin L”.

The terms “anti-Sema3A antibody” and “an antibody that binds to Sema3A” refer to an antibody that is capable of binding Sema3A with sufficient affinity such that the antibody is useful as a diagnostic and/or therapeutic agent in targeting Sema3A. In one embodiment, the extent of binding of an anti-Sema3A antibody to an unrelated, non-Sema3A protein is less than about 10%, less than about 5%, or less than about 2% of the binding of the antibody to Sema3A as measured, e.g., by standard ELISA procedure. In certain embodiments, an antibody that binds to Sema3A has a binding activity (EC50) of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g., from 10⁻⁹M to 10⁻¹³ M). In certain embodiments, an anti-Sema3A antibody binds to an epitope of Sema3A that is conserved among Sema3A from different species.

The term “antibody”, as used herein, is intended to refer to immunoglobulin molecules. Antibodies may comprise four polypeptide chains, two heavy (H) chains (about 50-70 kDa) and two light (L) chains (about 25 kDa) which are typically inter-connected by disulfide bonds. In particular embodiments, the antibody is composed of two identical pairs of polypeptide chains. The amino-terminal portion of each chain includes a “variable” region of about 100 to 110 or more amino acids primarily responsible for antigen recognition. The heavy chain variable region is abbreviated herein as VH, the light chain variable region is abbreviated herein as VL. The carboxyl-terminal portion of each chain defines a constant region primarily responsible for effector function. The heavy chain constant region can comprise e.g. three domains CH1, CH2 and CH3. The light chain constant region is comprised of one domain (CL). The VH and VL regions can be further subdivided into regions of hypervariability, termed complementarity determining regions (CDR), interspersed with regions that are more conserved, termed framework regions (FR). Each VH and VL is typically composed of three CDRs and up to four FRs, arranged from amino-terminus to carboxy-terminus e.g., in the following order: FR1, CDR1, FR2, CDR2, FR3, CDR3, FR4.

As used herein, the term “Complementarity Determining Regions” (CDRs; e.g., CDR1, CDR2, and CDR3) refers to the amino acid residues of an antibody variable domain the presence of which are necessary for antigen binding. Each variable domain typically has three CDR regions identified as CDR1, CDR2 and CDR3. Each complementarity determining region may comprise amino acid residues from a “complementarity determining region” as defined by Kabat (Kabat et al., Sequences of Proteins of Immunological Interest, 5th Ed. Public Health Service, National Institutes of Health, Bethesda, MD. (1991)) and/or those residues from a “hypervariable loop” (Chothia and Lesk; J Mol Biol 196: 901-917 (1987)). In some instances, a complementarity determining region can include amino acids from both a CDR region defined according to Kabat and a hypervariable loop.

“Framework” or FR residues are those variable domain residues other than the hypervariable region residues.

The phrase “constant region” refers to the portion of the antibody molecule that confers effector functions.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

Immunoglobulins can be assigned to different classes depending on the amino acid sequence of the constant domain of their heavy chains. Heavy chains are classified as mu (μ), delta (Δ), gamma (γ), alpha (α), and epsilon (ε), and define the antibody's isotype as IgM, IgD, IgG, IgA, and IgE, respectively. In particular embodiments, the antibody according to the present disclosure is an IgG antibody. Several of these may be further divided into subclasses or isotypes, e.g. IgG1, IgG2, IgG3, IgG4, IgA1 and IgA2. In particular embodiments, the antibody according to the present disclosure is an IgG1, an IgG2, an IgG3 or an IgG4 antibody, more particularly an IgG1 or an IgG4 antibody. Different isotypes may have different effector functions. Human light chains are classified as kappa (κ) and lambda (λ) light chains. Within light and heavy chains, the variable and constant regions are joined by a “J” region of about 12 or more amino acids, with the heavy chain also including a “D” region of about 10 more amino acids. See generally, Fundamental Immunology, Ch. 7 (Paul, W., ed., 2nd ed. Raven Press, N.Y. (1989)).

A “functional fragment” or “antigen-binding antibody fragment” of an antibody/immunoglobulin hereby is defined as a fragment of an antibody/immunoglobulin (e.g., a variable region of an IgG) that retains the antigen-binding region. An “antigen-binding region” of an antibody typically is found in one or more hyper variable region(s) of an antibody, e.g., the CDR1, -2, and/or -3 regions; however, the variable “framework” regions can also play an important role in antigen binding, such as by providing a scaffold for the CDRs. Preferably, the “antigen-binding region” comprises at least amino acid residues 4 to 103 of the variable light (VL) chain and 5 to 109 of the variable heavy (VH) chain, more preferably amino acid residues 3 to 107 of VL and 4 to 111 of VH, and particularly preferred are the complete VL and VH chains (amino acid positions 1 to 109 of VL and 1 to 113 of VH; numbering according to WO 97/08320).

Nonlimiting examples of “functional fragments” or “antigen-binding antibody fragments” include Fab, Fab′, F(ab′)2, Fv fragments, domain antibodies (dAb), complementarity determining region (CDR) fragments, single-chain antibodies (scFv), single chain antibody fragments, diabodies, triabodies, tetrabodies, minibodies, linear antibodies (Zapata et al., Protein Eng., 8 (10): 1057-1062 (1995)); chelating recombinant antibodies, tribodies or bibodies, intrabodies, nanobodies, small modular immunopharmaceuticals (SMIPs), an antigen-binding-domain immunoglobulin fusion protein, a camelized antibody, a VHH containing antibody, or muteins or derivatives thereof, and polypeptides that contain at least a portion of an immunoglobulin that is sufficient to confer specific antigen binding to the polypeptide, such as a CDR sequence, as long as the antibody retains the desired biological activity; and multispecific antibodies such as bi- and tri-specific antibodies formed from antibody fragments (C. A. K Borrebaeck, editor (1995) Antibody Engineering (Breakthroughs in Molecular Biology), Oxford University Press; R. Kontermann & S. Duebel, editors (2001) Antibody Engineering (Springer Laboratory Manual), Springer Verlag). An antibody other than a “bispecific” or “bifunctional” antibody is understood to have each of its binding sites identical. The F(ab′)₂ or Fab may be engineered to minimize or completely remove the intermolecular disulfide interactions that occur between the C_{H1} and C_{L} domains. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two “Fv” fragments. An “Fv” fragment is the minimum antibody fragment that contains a complete antigen recognition and binding site. This region consists of a dimer of one heavy- and one light-chain variable domain in tight, non-covalent association. It is in this configuration that the three CDRs of each variable domain interact to define an antigen binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen.

“Single-chain Fv” or “sFv” or “scFv” antibody fragments comprise the VH and VL domains of antibody, wherein these domains are present in a single polypeptide chain.

Preferably, the Fv polypeptide further comprises a polypeptide linker between the VH and VL domains that enables the Fv to form the desired structure for antigen binding. For a review of Fvs see Pluckthun in The Pharmacology of Monoclonal Antibodies, vol. 113, Rosenburg and Moore eds., Springer-Verlag, New York, pp. 269-315 (1994).

The Fab fragment also contains the constant domain of the light chain and the first constant domain (CH1) of the heavy chain. Fab fragments differ from Fab′ fragments by the addition of a few residues at the carboxyl terminus of the heavy chain CH1 domain including one or more cysteine residues from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteine residues between them.

The term “mutein” or “variant” can be used interchangeably and refers to an antibody or antigen-binding fragment that contains at least one amino acid substitution, deletion, or insertion in the variable region or the portion equivalent to the variable region, provided that the mutein or variant retains the desired binding affinity or biological activity. Variants of the antibodies or antigen-binding antibody fragments contemplated in the disclosure are molecules in which the binding activity of the antibody or antigen-binding antibody fragment is maintained.

A “chimeric antibody” or antigen-binding fragment thereof is defined herein as one, wherein the variable domains are derived from a non-human origin and some or all constant domains are derived from a human origin.

“Humanized antibodies” contain CDR regions derived from a non-human species, such as mouse, that have, for example, been engrafted, along with any necessary framework back-mutations, into human sequence-derived V regions. Thus, for the most part, humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired specificity, affinity, and capacity. See, for example, U.S. Pat. Nos. 5,225,539; 5,585,089; 5,693,761; 5,693,762; 5,859,205, each herein incorporated by reference. In some instances, framework residues of the human immunoglobulin are replaced by corresponding non-human residues (see, for example, U.S. Pat. Nos. 5,585,089; 5,693,761; 5,693,762, each herein incorporated by reference). Furthermore, humanized antibodies may comprise residues that are not found in the recipient antibody or in the donor antibody. These modifications are made to further refine antibody performance (e.g., to obtain desired affinity). In general, the humanized antibody will comprise substantially all of at least one, and typically two, variable domains, in which all or substantially all of the hypervariable regions correspond to those of a non-human immunoglobulin and all or substantially all of the framework regions are those of a human immunoglobulin sequence. The humanized antibody optionally also will comprise at least a portion of an immunoglobulin constant region (Fc), typically that of a human immunoglobulin. For further details see Jones et al., Nature 331:522-25 (1986); Riechmann et al., Nature 332:323-27 (1988); and Presta, Curr. Opin. Struct. Biol. 2:593-96 (1992), each herein incorporated by reference.

“Human antibodies” or “fully human antibodies” comprise human derived CDRs, i.e. CDRs of human origin. Fully human antibodies may comprise a low number of germline deviations compared with the closest human germline reference determined based on the IMGT database (http://www.imgt.org). For example, a fully human antibody according to the current disclosure may comprise up to 1, 2, 3, 4, 5, 6, 7, 8, 9 or 10 germline deviations in the CDRs compared with the closest human germline reference. Fully human antibodies can be developed from human derived B cells by cloning techniques in combination with a cell enrichment or immortalization step. The majority of fully human antibodies, however, are isolated either from immunized mice transgenic for the human IgG locus or from sophisticated combinatorial libraries by phage display (Bruggemann M., Osborn M. J., Ma B., Hayre J., Avis S., Lundstrom B. and Buelow R., Human Antibody Production in Transgenic Animals, Arch Immunol Ther Exp (Warsz.) 63 (2015), 101-108; Carter P. J., Potent antibody therapeutics by design, Nat Rev Immunol 6 (2006), 343-357; Frenzel A., Schirrmann T. and Hust M., Phage display-derived human antibodies in clinical development and therapy, MAbs 8 (2016), 1177-1194; Nelson A. L., Dhimolea E. and Reichert J. M., Development trends for human monoclonal antibody therapeutics, Nat Rev Drug Discov 9 (2010), 767-774.)).

Several techniques are available to generate fully human antibodies (cf. WO2008/112640 A3). Cambridge Antibody Technologies (CAT) and Dyax have obtained antibody cDNA sequences from peripheral B cells isolated from immunized humans and devised phage display libraries for the identification of human variable region sequences of a particular specificity. Briefly, the antibody variable region sequences are fused either with the Gene III or Gene VIII structure of the M13 bacteriophage. These antibody variable region sequences are expressed either as Fab or single chain Fv (scFv) structures at the tip of the phage carrying the respective sequences. Through rounds of a panning process using different levels of antigen binding conditions (stringencies), phages expressing Fab or scFv structures that are specific for the antigen of interest can be selected and isolated. The antibody variable region cDNA sequences of selected phages can then be elucidated using standard sequencing procedures. These sequences may then be used for the reconstruction of a full antibody having the desired isotype using established antibody engineering techniques. Antibodies constructed in accordance with this method are considered fully human antibodies (including the CDRs). In order to improve the immunoreactivity (antigen binding affinity and specificity) of the selected antibody, an in vitro maturation process can be introduced, including a combinatorial association of different heavy and light chains, deletion/addition/mutation at the CDR3 of the heavy and light chains (to mimic V-J, and V-D-J recombination), and random mutations (to mimic somatic hypermutation). An example of a “fully human” antibody generated by this method is the anti-tumor necrosis factor α antibody, Humira (adalimumab).

“Human Engineered™” antibodies generated by altering the parent sequence according to the methods set forth in Studnicka et al., U.S. Pat. No. 5,766,886.

An antibody of the disclosure may be derived from a recombinant antibody gene library. The development of technologies for making repertoires of recombinant human antibody genes, and the display of the encoded antibody fragments on the surface of filamentous bacteriophage, has provided a recombinant means for directly making and selecting human antibodies, which also can be applied to humanized, chimeric, murine or mutein antibodies. The antibodies produced by phage technology are produced as antigen binding fragments—usually Fv or Fab fragments—in bacteria and thus lack effector functions. Effector functions can be introduced by one of two strategies: The fragments can be engineered either into complete antibodies for expression in mammalian cells, or into bispecific antibody fragments with a second binding site capable of triggering an effector function. Typically, heavy chain VH-CH1 and light chain VL-CL of antibodies are separately cloned by PCR and recombined randomly in combinatorial phage display libraries, which can then be selected for binding to a particular antigen. The Fab fragments are expressed on the phage surface, i.e., physically linked to the genes that encode them. Thus, selection of Fab by antigen binding co-selects for the Fab encoding sequences, which can be amplified subsequently. By several rounds of antigen binding and re-amplification, a procedure termed panning, Fab specific for the antigen are enriched and finally isolated.

A variety of procedures have been described for human antibodies deriving from phage-display libraries. Such libraries may be built on a single master framework, into which diverse in vivo-formed (i. e. human-derived) CDRs are allowed to recombine as described by Carlsson and Söderlind Exp. Rev. Mol. Diagn. 1 (1), 102-108 (2001), Söderlin et al., Nat. Biotech. 18, 852-856 (2000) and U.S. Pat. No. 6,989,250. Alternatively, such an antibody library may be based on amino acid sequences that have been designed in silico and encoded by nucleic acids that are synthetically created. In silico design of an antibody sequence is achieved, for example, by analyzing a database of human sequences and devising a polypeptide sequence utilizing the data obtained therefrom. Methods for designing and obtaining in silico-created sequences are described, for example, in Knappik et al., J. Mol. Biol. (2000) 296:57; Krebs et al., J. Immunol. Methods. (2001) 254:67; and U.S. Pat. No. 6,300,064. For a review of phage display screening (for example see Hoet R M et al, Nat Biotechnol 2005; 23(3):344-8), the well-established hybridoma technology (for example see Köhler and Milstein Nature. 1975 Aug. 7; 256(5517):495-7), or immunization of mice inter alia immunization of hMAb mice (e.g. VelocImmune Mouse®).

The term “monoclonal antibody” as used herein refers to an antibody obtained from a population of substantially homogeneous antibodies, i.e., the individual antibodies comprising the population are identical except for possible mutations, e.g., naturally occurring mutations, that may be present in minor amounts. Thus, the term “monoclonal” indicates the character of the antibody as not being a mixture of discrete antibodies. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins. The term “monoclonal” is not to be construed as to require production of the antibody by any particular method. For example, the monoclonal antibodies to be used may be made by the hybridoma method first described by Kohler et al., Nature, 256: 495 [1975, or may be made by recombinant DNA methods (see, e.g., U.S. Pat. No. 4,816,567). The “monoclonal antibodies” may also be recombinant, chimeric, humanized, human, Human Engineered™, or antibody fragments, for example.

An “isolated” antibody is one that has been identified and separated from a component of the cell that expressed it. Contaminant components of the cell are materials that would interfere with diagnostic or therapeutic uses of the antibody, and may include enzymes, hormones, and other proteinaceous or non-proteinaceous solutes.

An “isolated” nucleic acid is one that has been identified and separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

As used herein, an antibody “binds specifically to”, is “specific to/for” or “specifically recognizes” an antigen of interest, e.g. Sema3A, is one that binds the antigen with sufficient affinity such that the antibody is useful as a therapeutic agent in targeting a cell or tissue expressing the antigen. The term “specifically recognizes” or “binds specifically to” or is “specific to/for” a particular polypeptide or an epitope on a particular polypeptide target as used herein can be exhibited, for example, by an antibody, or antigen-binding fragment thereof, having a monovalent K_{D} for the antigen of less than about 10⁻⁴ M, alternatively less than about 10⁻⁵ M, alternatively less than about 10⁻⁶ M, alternatively less than about 10⁻⁷ M, alternatively less than about 10⁻⁸ M, alternatively less than about 10⁻⁹ M, alternatively less than about 10⁻¹⁰ M, alternatively less than about 10⁻¹¹ M, alternatively less than about 10⁻¹² M, or less.

An antibody “binds selectively to,” is “selective to/for” or “selectively recognizes” an antigen if such antibody is able to discriminate between such antigen and one or more reference antigen(s). In particular, an antibody that “binds selectively to” an antigen does not significantly cross-react with proteins other than orthologs and variants (e.g. mutant forms, splice variants, or proteolytically truncated forms) of the aforementioned antigen target. In its most general form, “selective binding”, “binds selectively to”, is “selective to/for” or “selectively recognizes” is referring to the ability of the antibody to discriminate between the antigen of interest and an unrelated antigen, as determined, for example, in accordance with one of the following methods. Such methods comprise but are not limited to surface plasmon resonance (SPR), Western blots, ELISA-, RIA-, ECL-, IRMA-tests and peptide scans. For example, a standard ELISA assay can be carried out. The scoring may be carried out by standard color development (e.g. secondary antibody with horseradish peroxidase and tetramethyl benzidine with hydrogen peroxide). The reaction in certain wells is scored by the optical density, for example, at 450 nm. Typical background (=negative reaction) may be 0.1 OD; typical positive reaction may be 1 OD. This means the difference positive/negative is more than 5-fold, 10-fold, 50-fold, and preferably more than 100-fold. Typically, determination of binding selectivity is performed by using not a single reference antigen, but a set of about three to five unrelated antigens, such as milk powder, BSA, transferrin or the like.

“Binding affinity” or “affinity” refers to the strength of the total sum of non-covalent interactions between a single binding site of a molecule and its binding partner. Unless indicated otherwise, as used herein, “binding affinity” refers to intrinsic binding affinity which reflects a 1:1 interaction between members of a binding pair (e.g. an antibody and an antigen). The dissociation constant “K_{D}” is commonly used to describe the affinity between a molecule (such as an antibody) and its binding partner (such as an antigen) i.e. how tightly a ligand binds to a particular protein. Ligand-protein affinities are influenced by non-covalent intermolecular interactions between the two molecules. Affinity can be measured by common methods known in the art, including those described herein. In one embodiment, the “K_{D}” or “K_{D} value” according to this disclosure is measured by using surface plasmon resonance assays using a Biacore T200 instrument (GE Healthcare Biacore, Inc.). Other suitable devices are BIACORE T100, BIACORE®-2000, BIACORe 4000, a BIACORE®-3000 (BIAcore, Inc., Piscataway, NJ), or ProteOn XPR36 instrument (Bio-Rad Laboratories, Inc.).

As used herein, the term “epitope” includes any protein determinant capable of specific binding to an immunoglobulin or T-cell receptor. Epitopic determinants usually consist of chemically active surface groupings of molecules such as amino acids or sugar side chains, or combinations thereof and usually have specific three-dimensional structural characteristics, as well as specific charge characteristics.

An “antibody that binds to the same epitope” as a reference antibody or “an antibody which competes for binding” to a reference antibody refers to an antibody that blocks binding of the reference antibody to its antigen in a competition assay by 10%, 20%, 30%, 40%, 50% or more, and conversely, the reference antibody blocks binding of the antibody to its antigen in a competition assay by 10%, 20%, 30%, 40%, 50% or more.

The term “maturated antibodies” or “maturated antigen-binding fragments” such as maturated Fab variants or “optimized” variants includes derivatives of an antibody or antibody fragment exhibiting stronger binding—i. e. binding with increased affinity—to a given antigen such as the extracellular domain of a target protein. Maturation is the process of identifying a small number of mutations within the six CDRs of an antibody or antibody fragment leading to this affinity increase. The maturation process is the combination of molecular biology methods for introduction of mutations into the antibody and screening for identifying the improved binders.

“Percent (%) sequence identity” with respect to a reference polynucleotide or polypeptide sequence, respectively, is defined as the percentage of nucleic acid or amino acid residues, respectively, in a candidate sequence that are identical with the nucleic acid or amino acid residues, respectively, in the reference polynucleotide or polypeptide sequence, respectively, after aligning the sequences and introducing gaps, if necessary, to achieve the maximum percent sequence identity. Conservative substitutions are not considered as part of the sequence identity. Preferred are un-gapped alignments. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

“Sequence homology” indicates the percentage of amino acids that either is identical or that represent conservative amino acid substitutions.

An “antagonistic” antibody or a “blocking” antibody is one which significantly inhibits (either partially or completely) a biological activity of the antigen it binds. In particular embodiments, the antibody or antigen-binding fragment according to the present disclosure is a Sema3A blocking antibody or antigen-binding fragment thereof.

The term “antibody conjugate” refers to an antibody conjugated to one or more molecules including drugs—in which case the antibody conjugate is referred to as “antibody-drug conjugate” (“ADC”)—and high molecular weight molecules such as peptides or proteins.

The term “antibody-drug conjugate” or “ADC” refers to an antibody conjugated to one or more cytotoxic or cytostatic agents, such as a chemotherapeutic agent, a drug, a growth inhibitory agent, a toxin (e.g., a protein toxin, an enzymatically active toxin of bacterial, fungal, plant, or animal origin, or fragments thereof), or a radioactive isotope (i.e., a radioconjugate). Immunoconjugates have been used for the local delivery of cytotoxic agents, i.e., drugs that kill or inhibit the growth or proliferation of cells, in the treatment of cancer (e.g. Liu et al., Proc Natl. Acad. Sci. (1996), 93, 8618-8623)). Immunoconjugates allow for the targeted delivery of a drug moiety to a tumor, and intracellular accumulation therein, where systemic administration of unconjugated drugs may result in unacceptable levels of toxicity to normal cells and/or tissues. Toxins used in antibody-toxin conjugates include bacterial toxins such as diphtheria toxin, plant toxins such as ricin, small molecule toxins such as geldanamycin. The toxins may exert their cytotoxic effects by mechanisms including tubulin binding, DNA binding, or topoisomerase inhibition.

Amino acids may be referred to herein by their commonly known three letter symbols or by the one-letter symbols recommended by the IUPAC-IUB Biochemical Nomenclature Commission. Nucleotides, likewise, may be referred to by their commonly accepted single-letter codes.

The term “vector”, as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The terms “host cell”, “host cell line”, and “host cell culture” are used interchangeably and refer to cells into which at least one exogenous nucleic acid has been introduced, including the progeny of such cells. Host cells include “transformants” and “transformed cells”, “transfectants” and “transfected cells” and “transduced cells” which include the primary transformed/transfected/transduced cell and progeny derived therefrom without regard to the number of passages. Progeny may not be completely identical in nucleic acid content to a parent cell but may contain mutations. Mutant progeny that have the same function or biological activity as screened or selected for in the originally transformed cell are included herein.

As used herein, the phrase “therapeutically effective amount” is meant to refer to an amount of therapeutic or prophylactic antibody that would be appropriate to elicit the desired therapeutic or prophylactic effect or response, including alleviating some or all of such symptoms of disease or reducing the predisposition to the disease, when administered in accordance with the desired treatment regimen.

The term “pharmaceutical formulation”/“pharmaceutical composition” refers to a preparation which is in such form as to permit the biological activity of an active ingredient contained therein to be effective, and which contains no additional components which are unacceptably toxic to a subject to which the formulation would be administered.

### Antibodies According to the Present Disclosure

In one aspect the present disclosure relates to an isolated antibody or antigen-binding fragment thereof binding to human Sema3A, wherein said isolated antibody or antigen-binding fragment thereof binds to human Sema3A of the sequence of SEQ ID NO: 600 with a dissociation constant (KD)≤50 nM, ≤20 nM, ≤10 nM, ≤1 nM, or ≤0.1 nM. In particular embodiments, the isolated antibody or antigen-binding fragment thereof binds to the His-tagged human Sema3A domain of SEQ ID NO: 582 with a dissociation constant (KD)≤50 nM, ≤20 nM, ≤10 nM, ≤1 nM, or ≤0.1 nM.

In another aspect the present disclosure relates to an isolated antibody or antigen-binding fragment thereof binding to human Sema3A, wherein said isolated antibody or antigen-binding fragment thereof cross-reacts with mouse, cynomolgus, rat, pig and/or dog Sema3A, particularly wherein said isolated antibody or antigen-binding fragment thereof binds to mouse, cynomolgus, rat, pig and/or dog Sema3A with a dissociation constant (KD)≤50 nM, ≤20 nM, ≤10 nM, ≤1 nM, or ≤0.1 nM.

In particular such embodiments, said affinities are to mouse Sema3A of SEQ ID NO: 601, to cynomolgus Sema3A of SEQ ID NO: 602, to rat Sema3A of SEQ ID NO: 603, to pig Sema3A of SEQ ID NO: 604 and to dog Sema3A of SEQ ID NO: 605. In particular embodiments, said affinities are to His-tagged mouse Sema3A domain of SEQ ID NO: 583, to His-tagged cynomolgus Sema3A domain of SEQ ID NO: 586, to His-tagged rat Sema3A domain of SEQ ID NO: 584, to His-tagged pig Sema3A domain of SEQ ID NO: 587 and to His-tagged dog Sema3A domain of SEQ ID NO: 585.

In another aspect the present disclosure relates to an isolated antibody or antigen-binding fragment thereof binding to human Sema3A, wherein said isolated antibody or antigen-binding fragment thereof binds to human Sema3A with a binding activity as measured by surface plasmon resonance (SPR) of ≥60%, ≥70%, ≥80%, or ≥90%. In particular embodiments, the isolated antibody or antigen-binding fragment thereof bids to human Sema3A of the sequence of SEQ ID NO: 600 with a binding activity as measured by surface plasmin resonance (SPR) of ≥60%, ≥70%, ≥80%, or ≥90%. In particular embodiments, the isolated antibody or antigen-binding fragment thereof bids to His-tagged human Sema3A domain of the sequence of SEQ ID NO: 582 with a binding activity as measured by surface plasmin resonance (SPR) of ≥60%, ≥70%, ≥80%, or ≥90%.

In another aspect the present disclosure relates to an isolated antibody or antigen-binding fragment thereof binding to human Sema3A, wherein said isolated antibody or antigen-binding fragment thereof inhibits the activity of human Sema3A of the sequence of SEQ ID NO: 600 in an in vitro mesangial cell migration assay with an EC50 of ≤10 nM, ≤5 nM, ≤2.5 nM, or ≤1 nM.

In particular, the isolated antibody or antigen-binding fragment according to the present disclosure inhibits the activity of human Sema3A of the sequence of SEQ ID NO: 600 in an in vitro scratch assay using human primary mesangial cells and described in more detail in Example 9.

In another aspect the present disclosure relates to an isolated antibody or antigen-binding fragment thereof binding to human Sema3A, wherein said isolated antibody or antigen-binding fragment thereof inhibits the activity of human Sema3A of the sequence of SEQ ID NO: 600 in an in vitro growth cone collapse assay with an EC50 of ≤50 nM, ≤25 nM, ≤10 nM, or ≤5 nM.

In particular, the isolated antibody or antigen-binding fragment according to the present disclosure inhibits Sema3A-induced cytoskeletal collapse in an in vitro growth cone collapse assay using mouse dorsal root ganglion (DRG) neurons as described in more detail in Example 10. The in vitro growth cone assay described in Example 10 is a modified version of the growth cone assay described in Mikule et al. (PMID: 12077190).

In another aspect the present disclosure relates to an isolated antibody or antigen-binding fragment thereof binding to human Sema3A, wherein said isolated antibody or antigen-binding fragment thereof inhibits the activity of human Sema3A of the sequence of SEQ ID NO: 600 in an in vitro HUVEC repulsion assay with an EC50 of ≤1 nM, or ≤0.3 nM, ≤0.1 nM, ≤0.07 nM, ≤0.06 nM.

In particular, the isolated antibody or antigen-binding fragment according to the present disclosure inhibits Sema3A induced cell repulsion in an in vitro repulsion assay using Sema3A, of the sequence of SEQ ID NO: 600, expressing HEK293 cells seeded on a confluent monolayer of human umbilical vein endothelial cells (HUVEC) as described in Example 11.

In a further aspect, the present disclosure relates to an isolated antibody or antigen-binding fragment thereof binding to Sema3A, of the sequence of SEQ ID NO: 600, wherein said isolated antibody or antigen-binding fragment thereof exhibits an improved potency in HUVEC repulsion assay; i) wherein said isolated antibody or antigen-binding fragment thereof exhibits an improved potency in HUVEC repulsion assay in comparison to TPP-17755 with SEQ IDs 81, 85, 97, 98, or to TPP-11489 with SEQ IDs 1, 5, 17, 18, or to TPP-30788 with SEQ IDs 800, 804, 810, 811, or to TPP-30789 with SEQ IDs 814, 818, 824, 825, or to TPP-30790 with SEQ IDs 828, 832, 838, 839, or to TPP-30791 with SEQ IDs 842, 846, 852, 853; ii) wherein said isolated antibody or antigen-binding fragment thereof exhibits preferably a >400-fold, preferably a >50-fold, preferably >5-fold, preferably >2-fold increased potency against cellular Sema3A induced HUVEC repulsion based on the EC-50 values, in comparison to TPP-17755 with SEQ IDs 81, 85, 97, 98, or to TPP-11489 with SEQ IDs 1, 5, 17, 18, or to TPP-30788 with SEQ IDs 800, 804, 810, 811, or to TPP-30789 with SEQ IDs 814, 818, 824, 825, or to TPP-30790 with SEQ IDs 828, 832, 838, 839, or to TPP-30791 with SEQ IDs 842, 846, 852, 853; iii) wherein said isolated antibody or antigen-binding fragment thereof exhibits at least a 30% increased percent inhibition, preferably at least 50% increased percent inhibition of Sema3A in comparison to TPP-17755, to TPP-11489, to TPP-30788, to TPP-30789, TPP-30790, or to TPP-30791, with aforementioned sequences; iv) wherein said isolated antibody or antigen-binding fragment thereof has a two-digit picomolar activity against human Sema3A in vitro HUVEC repulsion assay, while prior art antibody potencies of TPP-17755, TPP-11489, TPP-30788, TPP-30789, TPP-30790, or TPP-30791, with aforementioned sequences, are in the three-digit picomolar or even nanomolar range; v) wherein said isolated antibody or antigen-binding fragment thereof inhibits the activity of human Sema3A in an in vitro HUVEC repulsion assay with an EC50 of ≤1 nM, or ≤0.3 nM, ≤0.1 nM, ≤0.07 nM, ≤0.06 nM, as described in Example 11.

The isolated antibody or antigen-binding fragment of the present disclosure show an improved potency in HUVEC repulsion assay compared to TPP-30788-TPP-30791 (BI clone I to IV), which might be due to a binding to a different epitope of human Sema3A.

In another aspect, the present disclosure relates to an isolated antibody or antigen-binding fragment thereof binding to Sema3A, wherein said isolated antibody or antigen-binding fragment thereof inhibits the activity of Sema3A in vivo, since the antibodies according to the present disclosure reduce Sema3A-induced urinary Albumin excretion. Thus in a further aspect, the present disclosure relates to an isolated antibody or antigen-binding fragment thereof binding to Sema3A, wherein said isolated antibody or antigen-binding fragment thereof exhibits an improved inhibitory activity of Sema3A in vivo, i) wherein said the antibodies exhibit an increased reduction of Sema3A-induced urinary Albumin excretion compared to TPP-30788 (BI clone I); ii) wherein said the antibodies exhibit an increased reduction of Sema3A-induced urinary Albumin excretion compared to TPP-17755 (Samsung); iii) wherein said the antibodies exhibit an increased reduction of Sema3A-induced urinary Albumin excretion compared to TPP-11489 (Chiome) as described in Example 12.

The isolated antibody or antigen-binding fragment of the present disclosure show an improved efficacy in an in vivo model for induced urinary Albumin excretion compared to TPP-30788-TPP-30791 (BI clone I to IV), which might be due to a binding to a different epitope of human Sema3A.

Thus, in a further aspect, the present disclosure relates to an isolated antibody or antigen-binding fragment thereof that binds to human Sema3A, wherein said isolated antibody or antigen-binding fragment thereof i) exhibits an increased stability (e.g. increased stress-stability when diluted in PBS to 25 mg/ml and incubated at 700 rpm and 40° C. for two weeks) compared to TPP-30788 (BI clone I); ii) wherein the increased stability exhibits an increased amount of monomeric anti-Sema3A antibody compared to TPP-30788 (BI clone I) measured by SEC; iii) wherein the increased stability exhibits a decreased percentage of the LC and HC of the anti-Sema3A antibody compared to TPP-30788 (BI clone I) measured by cGE, proving a reduced rate of degradation which is measured by the presence of remaining LC and HC, iv) wherein the increased stability exhibits that the amount of monomeric anti-Sema3A antibody is maintained, e.g. Δ % monomer=1 after the incubation at 40° C., 700 rpm for two weeks; v) wherein the increased stability exhibits that the amount of LC and HC of the anti-Sema3A antibody is maintained e.g. Δ % LC+HC<1 after the incubation at 40° C., 700 rpm for two weeks.

Thus, in a further aspect, the present disclosure relates to TPP-23298, that binds to human Sema3A, wherein said isolated antibody or antigen-binding fragment thereof i) exhibits an increased stability (e.g. increased stress-stability when diluted in PBS to 25 mg/ml and incubated at 700 rpm and 40° C. for two weeks) compared to TPP-30788 (BI clone I); ii) wherein the increased stability exhibits an increased amount of monomeric anti-Sema3A antibody compared to TPP-30788 (BI clone I) measured by SEC; iii) wherein the increased stability exhibits a decreased percentage of the LC and HC of the anti-Sema3A antibody compared to TPP-30788 (BI clone I) measured by cGE, proving a reduced rate of degradation which is measured by the presence of remaining LC and HC, iv) wherein the increased stability exhibits that the amount of monomeric anti-Sema3A antibody is maintained, e.g. Δ % monomer=1 after the incubation at 40° C., 700 rpm for two weeks; v) wherein the increased stability exhibits that the amount of LC and HC of the anti-Sema3A antibody is maintained e.g. Δ % LC+HC<1 after the incubation at 40° C., 700 rpm for two weeks.

Thus, in a further aspect, the present disclosure relates to an isolated antibody or antigen-binding fragment thereof that binds to human Sema3A, wherein said isolated antibody or antigen-binding fragment thereof i) exhibits an increased solubility; ii) wherein the increased solubility is measured in mg/ml after concentration at 90% recovery; iii) wherein the solubility is increased compared to TPP-30788 (BI clone I); iv) wherein the solubility is increased ≤1 fold, ≤1.5 fold, ≤2 fold compared to TPP-30788 (BI clone I); v) wherein the increased solubility exhibits that the percentage of monomeric anti-Sema3A antibody is not increased after concentration e.g. Δ % monomer<1 measured by SEC.

Thus, in a further aspect, the present disclosure relates to TPP-23298, that binds to human Sema3A, wherein said isolated antibody or antigen-binding fragment thereof i) exhibits an increased solubility; ii) wherein the increased solubility is measured in mg/ml after concentration at 90% recovery; iii) wherein the solubility is increased compared to TPP-30788 (BI clone I); iv) wherein the solubility is increased ≤1 fold, ≤1.5 fold, ≤2 fold compared to TPP-30788 (BI clone I); v) wherein the increased solubility exhibits that the percentage of monomeric anti-Sema3A antibody is not increased after concentration e.g. Δ % monomer<1 measured by SEC.

Thus, in a further aspect, the present disclosure relates to an isolated antibody or antigen-binding fragment thereof that binds to human Sema3A, wherein said isolated antibody or antigen-binding fragment thereof i) exhibits an increased viscosity compared to water or PBS; ii) exhibits a reduced viscosity in PBS compared to TPP-30788 (BI clone I); iii) wherein the viscosity is measured by a Viscosizer and exhibits a cP value of 5.1 (150 mg/ml).

Thus, in a further aspect, the present disclosure relates to TPP-23298, that binds to human Sema3A, wherein said isolated antibody or antigen-binding fragment thereof i) exhibits an increased viscosity compared to water or PBS; ii) exhibits a reduced viscosity in PBS compared to TPP-30788 (BI clone I); iii) wherein the viscosity is measured by a Viscosizer and exhibits a cP value of 5.1 (150 mg/ml).

In particular the isolated antibody or antigen-binding fragment according to the present disclosure shows a much higher solubility and stability, is more resistant to heat stress and is less viscous in PBS buffer than TPP-30788 as described in Example 17.

In particular TPP-23298 shows a much higher solubility and stability, is more resistant to heat stress and is less viscous in PBS buffer than TPP-30788 as described in Example 17.

In another aspect the present disclosure relates to an isolated antibody or antigen-binding fragment thereof binding to human Sema3A, which can be produced with high titers in mammalian cells; i) wherein high titer is ≤200 mg/L as described in Example 16.

In another aspect the present disclosure relates to an isolated antibody or antigen-binding fragment thereof binding to human Sema3A, wherein the antibody exhibits a higher binding selectivity to active Sema3A (TPP-13211) over cleaved Sema3A TPP-19068; i) wherein the antibody exhibits a higher binding selectivity to active Sema3A (TPP-13211) compared to the binding selectivity of TPP-30788-TPP-30791 to active Sema3A, as described in Example 8.

In another aspect the present disclosure relates to TPP-23298 binding to human Sema3A, wherein the antibody exhibits a higher binding selectivity to active Sema3A (TPP-13211) over cleaved Sema3A TPP-19068; i) wherein the antibody exhibits a higher binding selectivity to active Sema3A (TPP-13211) compared to the binding selectivity of TPP-30788-TPP-30791 to active Sema3A, as described in Example 8.

In another aspect the present disclosure relates to an isolated antibody or antigen-binding fragment thereof binding to human Sema3A, wherein the antibody binds a different epitope on Sema3A compared to TPP-30788; i) wherein the epitope binding is measured in SPR assay, as described in Example 5a. All antibodies binding the same epitope and competing with the binding of the isolated antibody or antigen-binding fragment according to the present disclosure are comprised by the present disclosure.

In another aspect the present disclosure relates to TPP-23298 binding to human Sema3A, wherein the antibody binds a different epitope on Sema3A compared to TPP-30788; i) wherein the epitope binding is measured in SPR assay, as described in Example 5a. All antibodies binding the same epitope and competing with the binding of the isolated antibody or antigen-binding fragment according to the present disclosure are comprised by the present disclosure.

In another aspect the present disclosure relates to an isolated antibody or antigen-binding fragment thereof that competes with the isolated antibody or antigen-binding fragment according to any one of the preceding claims for binding to Sema3A and wherein the isolated antibody or antigen-binding fragment does not compete with the binding of an antibody with the SEQ IDs NO 800, NO 804, NO 810 or NO 811 to Sema3A.

The isolated antibody or antigen-binding fragment according to the present disclosure may exhibit any combination of the above described characteristics.

The isolated antibody or antigen-binding fragment according to the present disclosure is a Sema3A blocking antibody or antigen-binding fragment thereof. In particular embodiments, the antibody binds specifically and more particularly selectively to the Sema3A domain of Semaphorin3A and interferes with the interaction of its receptor neuropilin-1.

In particular embodiments, the isolated antibody or antigen-binding fragment thereof according to the present disclosure cross-reacts with mouse, cynomolgus, rat, pig and/or dog Sema3A, particularly having an affinity to mouse, cynomolgus, rat, pig and/or dog Sema3A that is less than 100-fold, particularly less than 50-fold, more particularly less than 25-fold, even more particularly less than 10-fold and most particularly less than 5-fold different to that to human Sema3A.

In particular embodiments, the isolated antibody or antigen-binding fragment thereof according to the present disclosure does not significantly cross-react with human Sema3B, Sema3C, Sema3D, Sema3E, Sema3F and/or Sema3G. In particular, the isolated antibody or antigen-binding fragment thereof does not significantly cross-react with human Sema3G.

In particular embodiments, the isolated antibody or antigen-binding fragment thereof according to the present disclosure inhibits Sema3A-induced albuminuria and/or proteinuria.

In particular embodiments, the isolated antibody or antigen-binding fragment thereof according to the present disclosure inhibits Sema3A-induced fibrosis.

In particular embodiments, the isolated antibody or antigen-binding fragment according to the present disclosure comprises a heavy chain variable domain that is at least 90%, at least 95%, at least 98%, at least 99%, or 100% identical to SEQ ID NO: 141, and a light chain variable domain that is at least 90%, at least 95%, at least 98%, at least 99%, or 100% identical to SEQ ID NO: 145.

In particular other embodiments, the isolated antibody or antigen-binding fragment according to the present disclosure comprises a heavy chain variable domain that is at least 90%, at least 95%, at least 98%, at least 99%, or 100% identical to SEQ ID NO: 61, and a light chain variable domain that is at least 90%, at least 95%, at least 98%, at least 99%, or 100% identical to SEQ ID NO: 65.

In particular embodiments, the isolated antibody or antigen-binding fragment according to the present disclosure comprises a heavy chain antigen-binding region that comprises an H-CDR3 comprising the sequence RDDYTSRDAFDX (SEQ ID NO: 594), wherein X is selected from the group consisting of Y and V. Particularly, X is Y.

In particular embodiments, the isolated antibody or antigen-binding fragment according to the present disclosure comprises a light chain antigen-binding region that comprises an L-CDR3 comprising the sequence X₁AWDDSLNX₂X₃X₄V (SEQ ID NO: 598), wherein X₁ is selected from the group consisting of A and H, wherein X₂ is selected from the group consisting of V, D, and G, in particular wherein X₂ is selected from the group consisting of V and D, wherein X₃ is selected from the group consisting of I and Y, and wherein X₄ is selected from the group consisting of P and V.

In particular embodiments, the isolated antibody or antigen-binding fragment according to the present disclosure comprises a heavy chain antigen-binding region that comprises an H-CDR3 as defined above and a light chain antigen-binding region that comprises an L-CDR3 as defined above.

In particular embodiments, the isolated antibody or antigen-binding fragment according to the present disclosure comprises a heavy chain antigen-binding region that comprises an H-CDR3 comprising the sequence SGYSSSWFDPDFDY (SEQ ID NO: 64).

In particular embodiments, the isolated antibody or antigen-binding fragment according to the present disclosure comprises a light chain antigen-binding region that comprises an L-CDR3 comprising the sequence X₁SYX₂GX₃NPYVV (SEQ ID NO: 599), wherein X1 is selected from the group consisting of S and Q; wherein X₂ is selected from the group consisting of E and A; and wherein X₃ is selected from the group consisting of P, I, and S. In particular, X₃ is selected from the group consisting of P and I.

In particular embodiments, the isolated antibody or antigen-binding fragment according to the present disclosure comprises a heavy chain antigen-binding region that comprises an H-CDR3 as defined above and a light chain antigen-binding region that comprises an L-CDR3 as defined above.

In particular embodiments, the isolated antibody or antigen-binding fragment according to the present disclosure comprises a heavy chain antigen-binding region that comprises an H-CDR1 comprising the sequence SYX₁MX₂ (SEQ ID NO: 588), wherein X1 is selected from G and A and wherein X₂ is selected from H, S and L. Particularly, the heavy chain antigen-binding region comprises an H-CDR1 comprising the sequence SYAMX (SEQ ID NO: 589), wherein X is selected from S and L.

In particular embodiments, the isolated antibody or antigen-binding fragment according to the present disclosure comprises a heavy chain antigen-binding region that comprises an H-CDR2 comprising the sequence AIGX₁GGDTYYADSVX₂G (SEQ ID NO: 590), wherein X₁ is selected from T and Y, and wherein X₂ is selected from K and M. Particularly, the heavy chain antigen-binding region comprises an H-CDR2 comprising the sequence AIGXGGDTYYADSVKG (SEQ ID NO: 591), wherein X is selected from T and Y.

In particular embodiments, the isolated antibody or antigen-binding fragment according to the present disclosure comprises a heavy chain antigen-binding region that comprises an H-CDR3 comprising the sequence RDDYTSRDAFDX (SEQ ID NO: 594), wherein X is selected from the group consisting of Y and V. Particularly, X is Y.

In particular embodiments, the isolated antibody or antigen-binding fragment according to the present disclosure comprises a heavy chain antigen-binding region that comprises an H-CDR1, an H-CDR2 and an H-CDR3 as defined above.

In particular embodiments, the isolated antibody or antigen-binding fragment according to the present disclosure comprises a light chain antigen-binding region that comprises an L-CDR1 comprising the sequence SGSSSNIGSNTVN (SEQ ID NO: 46).

In particular embodiments, the isolated antibody or antigen-binding fragment according to the present disclosure comprises a light chain antigen-binding region that comprises an L-CDR2 comprising the sequence YDDLXPS (SEQ ID NO: 596), wherein X is selected from L and R. Particularly, the light chain antigen-binding region comprises an L-CDR2 comprising the sequence YDDLRPS (SEQ ID NO: 127).

In particular embodiments, the isolated antibody or antigen-binding fragment according to the present disclosure comprises a light chain antigen-binding region that comprises an L-CDR3 comprising the sequence X₁AWDDSLNX₂X₃X₄V (SEQ ID NO: 598), wherein X1 is selected from the group consisting of A and H, wherein X₂ is selected from the group consisting of V, D, and G, in particular wherein X₂ is selected from the group consisting of V and D, wherein X₃ is selected from the group consisting of I and Y, and wherein X₄ is selected from the group consisting of P and V.

In particular embodiments, the isolated antibody or antigen-binding fragment according to the present disclosure comprises a light chain antigen-binding region that comprises an L-CDR1, and L-CDR2 and an L-CDR3 as defined above.

In particular embodiments, the isolated antibody or antigen-binding fragment according to the present disclosure comprises a heavy chain antigen-binding region that comprises an H-CDR1, an H-CDR2 and an H-CDR3 as defined above and a light chain antigen-binding region that comprises an L-CDR1, and L-CDR2 and an L-CDR3 as defined above.

In particular such embodiments, the amino acid residue directly adjacent to the H-CDR1 at its 5′ end (corresponding to residue 30 of reference VH domain of SEQ ID NO: 121) is S or Y.

In particular other embodiments, the isolated antibody or antigen-binding fragment according to the present disclosure comprises a heavy chain antigen-binding region that comprises an H-CDR1 comprising the sequence SYEMN (SEQ ID NO: 62).

In particular embodiments, the isolated antibody or antigen-binding fragment according to the present disclosure comprises a heavy chain antigen-binding region that comprises an H-CDR2 comprising the sequence GISWNSGX₁IX₂YADSVKG (SEQ ID NO: 592), wherein X₁ is selected from W and S and X₂ is selected from G and D. Particularly, the heavy chain antigen-binding region comprises an H-CDR2 comprising the sequence GISWNSGWIXYADSVKG (SEQ ID NO: 593), wherein X is selected from G and D.

In particular embodiments, the isolated antibody or antigen-binding fragment according to the present disclosure comprises a heavy chain antigen-binding region that comprises an H-CDR3 comprising the sequence SGYSSSWFDPDFDY (SEQ ID NO: 64).

In particular embodiments, the isolated antibody or antigen-binding fragment according to the present disclosure comprises a heavy chain antigen-binding region that comprises an H-CDR1, an H-CDR2 and an H-CDR3 as defined above.

In particular embodiments, the isolated antibody or antigen-binding fragment according to the present disclosure comprises a light chain antigen-binding region that comprises an L-CDR1 comprising the sequence TGSSSXIGAGYDVH (SEQ ID NO: 595), wherein X is selected from N and D.

In particular embodiments, the isolated antibody or antigen-binding fragment according to the present disclosure comprises a light chain antigen-binding region that comprises an L-CDR2 comprising the sequence GXSNRPS (SEQ ID NO: 597), wherein X is selected from N and A.

In particular embodiments, the isolated antibody or antigen-binding fragment according to the present disclosure comprises a light chain antigen-binding region that comprises an L-CDR3 comprising the sequence X₁SYX₂GX₃NPYVV (SEQ ID NO: 599), wherein X1 is selected from the group consisting of S and Q, wherein X₂ is selected from the group consisting of E and A, and wherein X₃ is selected from the group consisting of P, I, and S. Particularly, X₃ is selected from the group consisting of P and I.

In particular embodiments, the isolated antibody or antigen-binding fragment according to the present disclosure comprises a light chain antigen-binding region that comprises an L-CDR1, and L-CDR2 and an L-CDR3 as defined above.

In particular embodiments, the isolated antibody or antigen-binding fragment according to the present disclosure comprises a heavy chain antigen-binding region that comprises an H-CDR1, an H-CDR2 and an H-CDR3 as defined above and a light chain antigen-binding region that comprises an L-CDR1, and L-CDR2 and an L-CDR3 as defined above.

In particular such embodiments, the three amino acid residues directly adjacent to the H-CDR1 at its 5′ end (corresponding to residues 28 to 30 of reference VH domain of SEQ ID NO: 101) are X₁FX₂, wherein X1 is selected form T and D and X₂ is selected from S and D.

In particular embodiments, the isolated antibody or antigen-binding fragment according to the present disclosure comprises:
i) a heavy chain antigen-binding region that comprises an H-CDR3 comprising SEQ ID NO: 44 and a light chain antigen-binding region that comprises an L-CDR3 comprising SEQ ID NO: 48; or
ii) a heavy chain antigen-binding region that comprises an H-CDR3 comprising SEQ ID NO: 64 and a light chain antigen-binding region that comprises an L-CDR3 comprising SEQ ID NO: 68; or.
iii) a heavy chain antigen-binding region that comprises an H-CDR3 comprising SEQ ID NO: 104 and a light chain antigen-binding region that comprises an L-CDR3 comprising SEQ ID NO: 108; or
iv) a heavy chain antigen-binding region that comprises an H-CDR3 comprising SEQ ID NO: 124 and a light chain antigen-binding region that comprises an L-CDR3 comprising SEQ ID NO: 128; or
v) a heavy chain antigen-binding region that comprises an H-CDR3 comprising SEQ ID NO: 144 and a light chain antigen-binding region that comprises an L-CDR3 comprising SEQ ID NO: 148; or
vi) a heavy chain antigen-binding region that comprises an H-CDR3 comprising SEQ ID NO: 164 and a light chain antigen-binding region that comprises an L-CDR3 comprising SEQ ID NO: 168; or
vii) a heavy chain antigen-binding region that comprises an H-CDR3 comprising SEQ ID NO: 184 and a light chain antigen-binding region that comprises an L-CDR3 comprising SEQ ID NO: 188; or
viii) a heavy chain antigen-binding region that comprises an H-CDR3 comprising SEQ ID NO: 204 and a light chain antigen-binding region that comprises an L-CDR3 comprising SEQ ID NO: 208; or
ix) a heavy chain antigen-binding region that comprises an H-CDR3 comprising SEQ ID NO: 224 and a light chain antigen-binding region that comprises an L-CDR3 comprising SEQ ID NO: 228; or
x) a heavy chain antigen-binding region that comprises an H-CDR3 comprising SEQ ID NO: 244 and a light chain antigen-binding region that comprises an L-CDR3 comprising SEQ ID NO: 248; or
xi) a heavy chain antigen-binding region that comprises an H-CDR3 comprising SEQ ID NO: 264 and a light chain antigen-binding region that comprises an L-CDR3 comprising SEQ ID NO: 268; or
xii) a heavy chain antigen-binding region that comprises an H-CDR3 comprising SEQ ID NO: 284 and a light chain antigen-binding region that comprises an L-CDR3 comprising SEQ ID NO: 288; or
xiii) a heavy chain antigen-binding region that comprises an H-CDR3 comprising SEQ ID NO: 304 and a light chain antigen-binding region that comprises an L-CDR3 comprising SEQ ID NO: 308; or
xiv) a heavy chain antigen-binding region that comprises an H-CDR3 comprising SEQ ID NO: 324 and a light chain antigen-binding region that comprises an L-CDR3 comprising SEQ ID NO: 328; or
xv) a heavy chain antigen-binding region that comprises an H-CDR3 comprising SEQ ID NO: 344 and a light chain antigen-binding region that comprises an L-CDR3 comprising SEQ ID NO: 348; or
xvi) a heavy chain antigen-binding region that comprises an H-CDR3 comprising SEQ ID NO: 364 and a light chain antigen-binding region that comprises an L-CDR3 comprising SEQ ID NO: 368; or
xvii) a heavy chain antigen-binding region that comprises an H-CDR3 comprising SEQ ID NO: 384 and a light chain antigen-binding region that comprises an L-CDR3 comprising SEQ ID NO: 388; or
xviii) a heavy chain antigen-binding region that comprises an H-CDR3 comprising SEQ ID NO: 404 and a light chain antigen-binding region that comprises an L-CDR3 comprising SEQ ID NO: 408; or
xix) a heavy chain antigen-binding region that comprises an H-CDR3 comprising SEQ ID NO: 424 and a light chain antigen-binding region that comprises an L-CDR3 comprising SEQ ID NO: 428; or
xx) a heavy chain antigen-binding region that comprises an H-CDR3 comprising SEQ ID NO: 444 and a light chain antigen-binding region that comprises an L-CDR3 comprising SEQ ID NO: 448; or
xxi) a heavy chain antigen-binding region that comprises an H-CDR3 comprising SEQ ID NO: 464 and a light chain antigen-binding region that comprises an L-CDR3 comprising SEQ ID NO: 468; or
xxii) a heavy chain antigen-binding region that comprises an H-CDR3 comprising SEQ ID NO: 484 and a light chain antigen-binding region that comprises an L-CDR3 comprising SEQ ID NO: 488; or
xxiii) a heavy chain antigen-binding region that comprises an H-CDR3 comprising SEQ ID NO: 504 and a light chain antigen-binding region that comprises an L-CDR3 comprising SEQ ID NO: 508; or
xxiv) a heavy chain antigen-binding region that comprises an H-CDR3 comprising SEQ ID NO: 524 and a light chain antigen-binding region that comprises an L-CDR3 comprising SEQ ID NO: 528; or
xxv) a heavy chain antigen-binding region that comprises an H-CDR3 comprising SEQ ID NO: 544 and a light chain antigen-binding region that comprises an L-CDR3 comprising SEQ ID NO: 548; or
xxvi) a heavy chain antigen-binding region that comprises an H-CDR3 comprising SEQ ID NO: 564 and a light chain antigen-binding region that comprises an L-CDR3 comprising SEQ ID NO: 568.

In particular embodiments, the isolated antibody or antigen-binding fragment according to the present disclosure comprises a heavy chain antigen-binding region that comprises an H-CDR1 comprising any one of SEQ ID NOs: 42, 62, 102, 122, 142, 162, 182, 202, 222, 242, 262, 282, 302, 322, 342, 362, 382, 402, 422, 442, 462, 482, 502, 522, 542, and 562.

In particular embodiments, the isolated antibody or antigen-binding fragment according to the present disclosure comprises a heavy chain antigen-binding region that comprises an H-CDR2 comprising any one of SEQ ID NOs: 43, 63, 103, 123, 143, 163, 183, 203, 223, 243, 263, 283, 303, 323, 343, 363, 383, 403, 423, 443, 463, 483, 503, 523, 543, and 563.

In particular embodiments, the isolated antibody or antigen-binding fragment according to the present disclosure comprises a light chain antigen-binding region that comprises an L-CDR1 comprising any one of SEQ ID NOs: 46, 66, 106, 126, 146, 166, 186, 206, 226, 246, 266, 286, 306, 326, 346, 366, 386, 406, 426, 446, 466, 486, 506, 526, 546, and 566.

In particular embodiments, the isolated antibody or antigen-binding fragment according to the present disclosure comprises a light chain antigen-binding region that comprises an L-CDR2 comprising any one of SEQ ID NOs: 47, 67, 107, 127, 147, 167, 187, 207, 227, 247, 267, 287, 307, 327, 347, 367, 387, 407, 427, 447, 467, 487, 507, 527, 547, and 567.

In particular embodiments, the isolated antibody or antigen-binding fragment according to the present disclosure comprises:
i) a heavy chain antigen-binding region that comprises an H-CDR1 comprising SEQ ID NO: 42, an H-CDR2 comprising SEQ ID NO: 43, and an H-CDR3 comprising SEQ ID NO: 44 and a light chain antigen-binding region that comprises an L-CDR1 comprising SEQ ID NO: 46, an L-CDR2 comprising SEQ ID NO: 47, and an L-CDR3 comprising SEQ ID NO: 48; or
ii) a heavy chain antigen-binding region that comprises an H-CDR1 comprising SEQ ID NO: 62, an H-CDR2 comprising SEQ ID NO: 63, and an H-CDR3 comprising SEQ ID NO: 64 and a light chain antigen-binding region that comprises an L-CDR1 comprising SEQ ID NO: 66, an L-CDR2 comprising SEQ ID NO: 67, and an L-CDR3 comprising SEQ ID NO: 68; or
iii) a heavy chain antigen-binding region that comprises an H-CDR1 comprising SEQ ID NO: 102, an H-CDR2 comprising SEQ ID NO: 103, and an H-CDR3 comprising SEQ ID NO: 104 and a light chain antigen-binding region that comprises an L-CDR1 comprising SEQ ID NO: 106, an L-CDR2 comprising SEQ ID NO: 107, and an L-CDR3 comprising SEQ ID NO: 108; or
iv) a heavy chain antigen-binding region that comprises an H-CDR1 comprising SEQ ID NO: 122, an H-CDR2 comprising SEQ ID NO: 123, and an H-CDR3 comprising SEQ ID NO: 124 and a light chain antigen-binding region that comprises an L-CDR1 comprising SEQ ID NO: 126, an L-CDR2 comprising SEQ ID NO: 127, and an L-CDR3 comprising SEQ ID NO: 128; or
v) a heavy chain antigen-binding region that comprises an H-CDR1 comprising SEQ ID NO: 142, an H-CDR2 comprising SEQ ID NO: 143, and an H-CDR3 comprising SEQ ID NO: 144 and a light chain antigen-binding region that comprises an L-CDR1 comprising SEQ ID NO: 146, an L-CDR2 comprising SEQ ID NO: 147, and an L-CDR3 comprising SEQ ID NO: 148; or
vi) a heavy chain antigen-binding region that comprises an H-CDR1 comprising SEQ ID NO: 162, an H-CDR2 comprising SEQ ID NO: 163, and an H-CDR3 comprising SEQ ID NO: 164 and a light chain antigen-binding region that comprises an L-CDR1 comprising SEQ ID NO: 166, an L-CDR2 comprising SEQ ID NO: 167, and an L-CDR3 comprising SEQ ID NO: 168; or
vii) a heavy chain antigen-binding region that comprises an H-CDR1 comprising SEQ ID NO: 182, an H-CDR2 comprising SEQ ID NO: 183, and an H-CDR3 comprising SEQ ID NO: 184 and a light chain antigen-binding region that comprises an L-CDR1 comprising SEQ ID NO: 186, an L-CDR2 comprising SEQ ID NO: 187, and an L-CDR3 comprising SEQ ID NO: 188; or
viii) a heavy chain antigen-binding region that comprises an H-CDR1 comprising SEQ ID NO: 202, an H-CDR2 comprising SEQ ID NO: 203, and an H-CDR3 comprising SEQ ID NO: 204 and a light chain antigen-binding region that comprises an L-CDR1 comprising SEQ ID NO: 206, an L-CDR2 comprising SEQ ID NO: 207, and an L-CDR3 comprising SEQ ID NO: 208; or
ix) a heavy chain antigen-binding region that comprises an H-CDR1 comprising SEQ ID NO: 222, an H-CDR2 comprising SEQ ID NO: 223, and an H-CDR3 comprising SEQ ID NO: 224 and a light chain antigen-binding region that comprises an L-CDR1 comprising SEQ ID NO: 226, an L-CDR2 comprising SEQ ID NO: 227, and an L-CDR3 comprising SEQ ID NO: 228; or
x) a heavy chain antigen-binding region that comprises an H-CDR1 comprising SEQ ID NO: 242, an H-CDR2 comprising SEQ ID NO: 243, and an H-CDR3 comprising SEQ ID NO: 244 and a light chain antigen-binding region that comprises an L-CDR1 comprising SEQ ID NO: 246, an L-CDR2 comprising SEQ ID NO: 247, and an L-CDR3 comprising SEQ ID NO: 248; or
xi) a heavy chain antigen-binding region that comprises an H-CDR1 comprising SEQ ID NO: 262, an H-CDR2 comprising SEQ ID NO: 263, and an H-CDR3 comprising SEQ ID NO: 264 and a light chain antigen-binding region that comprises an L-CDR1 comprising SEQ ID NO: 266, an L-CDR2 comprising SEQ ID NO: 267, and an L-CDR3 comprising SEQ ID NO: 268; or
xii) a heavy chain antigen-binding region that comprises an H-CDR1 comprising SEQ ID NO: 282, an H-CDR2 comprising SEQ ID NO: 283, and an H-CDR3 comprising SEQ ID NO: 284 and a light chain antigen-binding region that comprises an L-CDR1 comprising SEQ ID NO: 286, an L-CDR2 comprising SEQ ID NO: 287, and an L-CDR3 comprising SEQ ID NO: 288; or
xiii) a heavy chain antigen-binding region that comprises an H-CDR1 comprising SEQ ID NO: 302, an H-CDR2 comprising SEQ ID NO: 303, and an H-CDR3 comprising SEQ ID NO: 304 and a light chain antigen-binding region that comprises an L-CDR1 comprising SEQ ID NO: 306, an L-CDR2 comprising SEQ ID NO: 307, and an L-CDR3 comprising SEQ ID NO: 308; or
xiv) a heavy chain antigen-binding region that comprises an H-CDR1 comprising SEQ ID NO: 322, an H-CDR2 comprising SEQ ID NO: 323, and an H-CDR3 comprising SEQ ID NO: 324 and a light chain antigen-binding region that comprises an L-CDR1 comprising SEQ ID NO: 326, an L-CDR2 comprising SEQ ID NO: 327, and an L-CDR3 comprising SEQ ID NO: 328; or
xv) a heavy chain antigen-binding region that comprises an H-CDR1 comprising SEQ ID NO: 342, an H-CDR2 comprising SEQ ID NO: 343, and an H-CDR3 comprising SEQ ID NO: 344 and a light chain antigen-binding region that comprises an L-CDR1 comprising SEQ ID NO: 346, an L-CDR2 comprising SEQ ID NO: 347, and an L-CDR3 comprising SEQ ID NO: 348; or
xvi) a heavy chain antigen-binding region that comprises an H-CDR1 comprising SEQ ID NO: 362, an H-CDR2 comprising SEQ ID NO: 363, and an H-CDR3 comprising SEQ ID NO: 364 and a light chain antigen-binding region that comprises an L-CDR1 comprising SEQ ID NO: 366, an L-CDR2 comprising SEQ ID NO: 367, and an L-CDR3 comprising SEQ ID NO: 368; or
xvii) a heavy chain antigen-binding region that comprises an H-CDR1 comprising SEQ ID NO: 382, an H-CDR2 comprising SEQ ID NO: 383, and an H-CDR3 comprising SEQ ID NO: 384 and a light chain antigen-binding region that comprises an L-CDR1 comprising SEQ ID NO: 386, an L-CDR2 comprising SEQ ID NO: 387, and an L-CDR3 comprising SEQ ID NO: 388; or
xviii) a heavy chain antigen-binding region that comprises an H-CDR1 comprising SEQ ID NO: 402, an H-CDR2 comprising SEQ ID NO: 403, and an H-CDR3 comprising SEQ ID NO: 404 and a light chain antigen-binding region that comprises an L-CDR1 comprising SEQ ID NO: 406, an L-CDR2 comprising SEQ ID NO: 407, and an L-CDR3 comprising SEQ ID NO: 408; or
xix) a heavy chain antigen-binding region that comprises an H-CDR1 comprising SEQ ID NO: 422, an H-CDR2 comprising SEQ ID NO: 423, and an H-CDR3 comprising SEQ ID NO: 424 and a light chain antigen-binding region that comprises an L-CDR1 comprising SEQ ID NO: 426, an L-CDR2 comprising SEQ ID NO: 427, and an L-CDR3 comprising SEQ ID NO: 428; or
xx) a heavy chain antigen-binding region that comprises an H-CDR1 comprising SEQ ID NO: 442, an H-CDR2 comprising SEQ ID NO: 443, and an H-CDR3 comprising SEQ ID NO: 444 and a light chain antigen-binding region that comprises an L-CDR1 comprising SEQ ID NO: 446, an L-CDR2 comprising SEQ ID NO: 447, and an L-CDR3 comprising SEQ ID NO: 448; or
xxi) a heavy chain antigen-binding region that comprises an H-CDR1 comprising SEQ ID NO: 462, an H-CDR2 comprising SEQ ID NO: 463, and an H-CDR3 comprising SEQ ID NO: 464 and a light chain antigen-binding region that comprises an L-CDR1 comprising SEQ ID NO: 466, an L-CDR2 comprising SEQ ID NO: 467, and an L-CDR3 comprising SEQ ID NO: 468; or
xxii) a heavy chain antigen-binding region that comprises an H-CDR1 comprising SEQ ID NO: 482, an H-CDR2 comprising SEQ ID NO: 483, and an H-CDR3 comprising SEQ ID NO: 484 and a light chain antigen-binding region that comprises an L-CDR1 comprising SEQ ID NO: 486, an L-CDR2 comprising SEQ ID NO: 487, and an L-CDR3 comprising SEQ ID NO: 488; or
xxiii) a heavy chain antigen-binding region that comprises an H-CDR1 comprising SEQ ID NO: 502, an H-CDR2 comprising SEQ ID NO: 503, and an H-CDR3 comprising SEQ ID NO: 504 and a light chain antigen-binding region that comprises an L-CDR1 comprising SEQ ID NO: 506, an L-CDR2 comprising SEQ ID NO: 507, and an L-CDR3 comprising SEQ ID NO: 508; or
xxiv) a heavy chain antigen-binding region that comprises an H-CDR1 comprising SEQ ID NO: 522, an H-CDR2 comprising SEQ ID NO: 523, and an H-CDR3 comprising SEQ ID NO: 524 and a light chain antigen-binding region that comprises an L-CDR1 comprising SEQ ID NO: 526, an L-CDR2 comprising SEQ ID NO: 527, and an L-CDR3 comprising SEQ ID NO: 528; or
xxv) a heavy chain antigen-binding region that comprises an H-CDR1 comprising SEQ ID NO: 542, an H-CDR2 comprising SEQ ID NO: 543, and an H-CDR3 comprising SEQ ID NO: 544 and a light chain antigen-binding region that comprises an L-CDR1 comprising SEQ ID NO: 546, an L-CDR2 comprising SEQ ID NO: 547, and an L-CDR3 comprising SEQ ID NO: 548; or
xxvi) a heavy chain antigen-binding region that comprises an H-CDR1 comprising SEQ ID NO: 562, an H-CDR2 comprising SEQ ID NO: 563, and an H-CDR3 comprising SEQ ID NO: 564 and a light chain antigen-binding region that comprises an L-CDR1 comprising SEQ ID NO: 566, an L-CDR2 comprising SEQ ID NO: 567, and an L-CDR3 comprising SEQ ID NO: 568.

In particular embodiments, the isolated antibody or antigen-binding fragment according to the present disclosure comprises:
i) a variable heavy chain domain comprising SEQ ID NO: 41 and a variable light chain domain comprising SEQ ID NO: 45; or
ii) a variable heavy chain domain comprising SEQ ID NO: 61 and a variable light chain domain comprising SEQ ID NO: 65; or
iii) a variable heavy chain domain comprising SEQ ID NO: 101 and a variable light chain domain comprising SEQ ID NO: 105; or
iv) a variable heavy chain domain comprising SEQ ID NO: 121 and a variable light chain domain comprising SEQ ID NO: 125; or
v) a variable heavy chain domain comprising SEQ ID NO: 141 and a variable light chain domain comprising SEQ ID NO: 145; or
vi) a variable heavy chain domain comprising SEQ ID NO: 161 and a variable light chain domain comprising SEQ ID NO: 165; or
vii) a variable heavy chain domain comprising SEQ ID NO: 181 and a variable light chain domain comprising SEQ ID NO: 185; or
viii) a variable heavy chain domain comprising SEQ ID NO: 201 and a variable light chain domain comprising SEQ ID NO: 205; or
ix) a variable heavy chain domain comprising SEQ ID NO: 221 and a variable light chain domain comprising SEQ ID NO: 225; or
x) a variable heavy chain domain comprising SEQ ID NO: 241 and a variable light chain domain comprising SEQ ID NO: 245; or
xi) a variable heavy chain domain comprising SEQ ID NO: 261 and a variable light chain domain comprising SEQ ID NO: 265; or
xii) a variable heavy chain domain comprising SEQ ID NO: 281 and a variable light chain domain comprising SEQ ID NO: 285; or
xiii) a variable heavy chain domain comprising SEQ ID NO: 301 and a variable light chain domain comprising SEQ ID NO: 305; or
xiv) a variable heavy chain domain comprising SEQ ID NO: 321 and a variable light chain domain comprising SEQ ID NO: 325; or
xv) a variable heavy chain domain comprising SEQ ID NO: 341 and a variable light chain domain comprising SEQ ID NO: 345; or
xvi) a variable heavy chain domain comprising SEQ ID NO: 361 and a variable light chain domain comprising SEQ ID NO: 365; or
xvii) a variable heavy chain domain comprising SEQ ID NO: 381 and a variable light chain domain comprising SEQ ID NO: 385; or
xviii) a variable heavy chain domain comprising SEQ ID NO: 401 and a variable light chain domain comprising SEQ ID NO: 405; or
xix) a variable heavy chain domain comprising SEQ ID NO: 421 and a variable light chain domain comprising SEQ ID NO: 425; or
xx) a variable heavy chain domain comprising SEQ ID NO: 441 and a variable light chain domain comprising SEQ ID NO: 445; or
xxi) a variable heavy chain domain comprising SEQ ID NO: 461 and a variable light chain domain comprising SEQ ID NO: 465; or
xxii) a variable heavy chain domain comprising SEQ ID NO: 481 and a variable light chain domain comprising SEQ ID NO: 485; or
xxiii) a variable heavy chain domain comprising SEQ ID NO: 501 and a variable light chain domain comprising SEQ ID NO: 505; or
xxiv) a variable heavy chain domain comprising SEQ ID NO: 521 and a variable light chain domain comprising SEQ ID NO: 525; or
xxv) a variable heavy chain domain comprising SEQ ID NO: 541 and a variable light chain domain comprising SEQ ID NO: 545; or
xxvi) a variable heavy chain domain comprising SEQ ID NO: 561 and a variable light chain domain comprising SEQ ID NO: 565.

In particular embodiments, the isolated antibody according to the present disclosure is an IgG antibody. In particular such embodiments, the isolated antibody according to the present disclosure is an IgG1, IgG2, IgG3 or an IgG4 antibody. Most particularly, the isolated antibody according to the present disclosure is an IgG1 or an IgG4 antibody.

In particular embodiments, the isolated antibody according to the present disclosure comprises:
i) a heavy chain comprising SEQ ID NO: 57 and a light chain comprising SEQ ID NO: 58; or
ii) a heavy chain comprising SEQ ID NO: 77 and a light chain comprising SEQ ID NO: 78; or
iii) a heavy chain comprising SEQ ID NO: 117 and a light chain comprising SEQ ID NO: 118; or
iv) a heavy chain comprising SEQ ID NO: 137 and a light chain comprising SEQ ID NO: 138; or
v) a heavy chain comprising SEQ ID NO: 157 and a light chain comprising SEQ ID NO: 158; or
vi) a heavy chain comprising SEQ ID NO: 177 and a light chain comprising SEQ ID NO: 178; or
vii) a heavy chain comprising SEQ ID NO: 197 and a light chain comprising SEQ ID NO: 198; or
viii) a heavy chain comprising SEQ ID NO: 217 and a light chain comprising SEQ ID NO: 218; or
ix) a heavy chain comprising SEQ ID NO: 237 and a light chain comprising SEQ ID NO: 238; or
x) a heavy chain comprising SEQ ID NO: 257 and a light chain comprising SEQ ID NO: 258; or
xi) a heavy chain comprising SEQ ID NO: 277 and a light chain comprising SEQ ID NO: 278; or
xii) a heavy chain comprising SEQ ID NO: 297 and a light chain comprising SEQ ID NO: 298; or
xiii) a heavy chain comprising SEQ ID NO: 317 and a light chain comprising SEQ ID NO: 318; or
xiv) a heavy chain comprising SEQ ID NO: 337 and a light chain comprising SEQ ID NO: 338; or
xv) a heavy chain comprising SEQ ID NO: 357 and a light chain comprising SEQ ID NO: 358; or
xvi) a heavy chain comprising SEQ ID NO: 377 and a light chain comprising SEQ ID NO: 378; or
xvii) a heavy chain comprising SEQ ID NO: 397 and a light chain comprising SEQ ID NO: 398; or
xviii) a heavy chain comprising SEQ ID NO: 417 and a light chain comprising SEQ ID NO: 418; or
xix) a heavy chain comprising SEQ ID NO: 437 and a light chain comprising SEQ ID NO: 438; or
xx) a heavy chain comprising SEQ ID NO: 457 and a light chain comprising SEQ ID NO: 458; or
xxi) a heavy chain comprising SEQ ID NO: 477 and a light chain comprising SEQ ID NO: 478; or
xxii) a heavy chain comprising SEQ ID NO: 497 and a light chain comprising SEQ ID NO: 498; or
xxiii) a heavy chain comprising SEQ ID NO: 517 and a light chain comprising SEQ ID NO: 518; or
xxiv) a heavy chain comprising SEQ ID NO: 537 and a light chain comprising SEQ ID NO: 538; or
xxv) a heavy chain comprising SEQ ID NO: 557 and a light chain comprising SEQ ID NO: 558; or
xxvi) a heavy chain comprising SEQ ID NO: 577 and a light chain comprising SEQ ID NO: 578.

In particular embodiments, the antigen-binding fragment according to the present disclosure is an scFv, Fab, Fab′ fragment or a F(ab′)2 fragment.

In particular embodiments, the isolated antibody or antigen-binding fragment according to the present disclosure is a monoclonal antibody or antigen-binding fragment thereof.

In particular embodiments, the isolated antibody or antigen-binding fragment according to the present disclosure is a human, humanized or chimeric antibody or antigen-binding fragment thereof, more particularly a fully human antibody or antigen-binding fragment thereof.

In particular embodiments, the isolated antibody or antigen-binding fragment according to the present disclosure is a monospecific antibody. In particular other embodiments, the isolated antibody or antigen-binding fragment according to the present disclosure is a multispecific antibody that binds to Sema3A and at least one further antigen, such as a bispecific, trispecific or tetraspecific antibody.

In another aspect the present disclosure relates to an isolated antibody or antigen-binding fragment thereof that competes with the isolated antibody or antigen-binding fragment according to the present disclosure for binding to human Sema3A.

In a further aspect, the present disclosure relates to an antibody conjugate, comprising the isolated antibody or antigen binding fragment according to the present disclosure. For example, an antibody could be conjugated to a cytotoxic agent, an immunotoxin, a toxophore or a radioisotope. Also provided are anti-Sema3A antibodies conjugated to a detectable marker. Preferred markers are a radiolabel, an enzyme, a chromophore or a fluorophore. The antibody may also be conjugated to high molecular weight molecules such as peptides or proteins, such as interleukins.

The ADC according to the present disclosure comprises an anti-Sema3A antibody conjugated to one or more cytotoxic agents, such as chemotherapeutic agents or drugs, growth inhibitory agents, toxins (e.g., protein toxins, enzymatically active toxins of bacterial, fungal, plant, human or animal origin, or fragments thereof), or radioactive isotopes.

In one embodiment, the ADC according to the present disclosure comprises an anti-Sema3A antibody as described herein conjugated to one or more drugs, including but not limited to a maytansinoid (see U.S. Pat. Nos. 5,208,020, 5,416,064 and European Patent EP0425235); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof; an anthracycline such as daunomycin or doxorubicin; methotrexate; vindesine; a taxane such as docetaxel, paclitaxel, larotaxel, tesetaxel, and ortataxel; a trichothecene; and CC1065.

In another embodiment, the ADC according to the present disclosure comprises an anti-Sema3A antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alphasarcin, Aleurites fordii proteins, dianthin proteins, Phytolaca americana proteins (P API, P APII, and PAP-S), Momordica charantia inhibitor, curcin, crotin, Sapaonaria officinalis inhibitor, gelonin, mitogellin, restrictocin, phenomycin, enomycin, and the tricothecenes.

In another embodiment, the ACD according to the present disclosure comprises and anti-Sema3A antibody as described herein conjugated to a radioactive atom to form a radioconjugate. A variety of radioactive isotopes are available for the production of radioconjugates. Examples include 227Th, 225Ac, 211At, 131I, 125I, 90Y, 186Re, 188Re, 153Sm, 212Bi, 32P, 212Pb and radioactive isotopes of Lu. When the radioconjugate is used for detection, it may comprise a radioactive atom for scintigraphic studies, for example Tc99m, or a spin label for nuclear magnetic resonance (NMR) imaging, such as iodine-123 again, iodine-131, indium-111, fluorine-19, carbon-13, nitrogen-15, oxygen-17, gadolinium, manganese or iron.

Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene).

The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52: 12 7-131 (1992).

The ACD according to the present disclosure includes ADCs prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, IL., U.S.A)

Amino acid and nucleic acid sequences of preferred antibodies according to the present disclosure and three prior art antibodies are listed in Table 1 and Table 1A.

### Peptide Variants

Antibodies or antigen-binding fragments of the disclosure are not limited to the specific peptide sequences provided herein. Rather, the disclosure also embodies variants of these polypeptides. With reference to the instant disclosure and conventionally available technologies and references, the skilled worker will be able to prepare, test and utilize functional variants of the antibodies disclosed herein, while appreciating these variants having the ability to bind to Sema3A fall within the scope of the present disclosure.

A variant can include, for example, an antibody that has at least one altered complementary determining region (CDR) (hyper-variable) and/or framework (FR) (variable) domain/position, vis-à-vis a peptide sequence disclosed herein.

By altering one or more amino acid residues in a CDR or FR region, the skilled worker routinely can generate mutated or diversified antibody sequences, which can be screened against the antigen, for new or improved properties, for example.

A further preferred embodiment of the disclosure is an antibody or antigen-binding fragment thereof in which the VH and VL sequences are selected as shown in Table 1 and Table 1A. The skilled worker can use the data in Table 1 and Table 1A to design peptide variants that are within the scope of the present disclosure. It is preferred that variants are constructed by changing amino acids within one or more CDR regions; a variant might also have one or more altered framework regions. For example, a peptide FR domain might be altered where there is a deviation in a residue compared to a germline sequence.

Alternatively, the skilled worker could make the same analysis by comparing the amino acid sequences disclosed herein to known sequences of the same class of such antibodies, using, for example, the procedure described by Knappik A., et al., JMB 2000, 296:57-86.

Furthermore, variants may be obtained by using one antibody as starting point for further optimization by diversifying one or more amino acid residues in the antibody, preferably amino acid residues in one or more CDRs, and by screening the resulting collection of antibody variants for variants with improved properties. Particularly preferred is diversification of one or more amino acid residues in CDR3 of VL and/or VH. Diversification can be done e.g. by synthesizing a collection of DNA molecules using trinucleotide mutagenesis (TRIM) technology (Virnekäs B. et al., Nucl. Acids Res. 1994, 22: 5600.). Antibodies or antigen-binding fragments thereof include molecules with modifications/variations including but not limited to e.g. modifications leading to altered half-life (e.g. modification of the Fc part or attachment of further molecules such as PEG), altered binding affinity or altered ADCC or CDC activity.

### Conservative Amino Acid Variants

Polypeptide variants may be made that conserve the overall molecular structure of an antibody peptide sequence described herein. Given the properties of the individual amino acids, some rational substitutions will be recognized by the skilled worker. Amino acid substitutions, i.e., “conservative substitutions,” may be made, for instance, on the basis of similarity in polarity, charge, solubility, hydrophobicity, hydrophilicity, and/or the amphipathic nature of the residues involved.

For example, (a) nonpolar (hydrophobic) amino acids include alanine, leucine, isoleucine, valine, proline, phenylalanine, tryptophane, and methionine; (b) polar neutral amino acids include glycine, serine, threonine, cysteine, tyrosine, asparagine, and glutamine; (c) positively charged (basic) amino acids include arginine, lysine, and histidine; and (d) negatively charged (acidic) amino acids include aspartic acid and glutamic acid. Substitutions typically may be made within groups (a)-(d). In addition, glycine and proline may be substituted for one another based on their ability to disrupt α-helices. Similarly, certain amino acids, such as alanine, cysteine, leucine, methionine, glutamic acid, glutamine, histidine and lysine are more commonly found in α-helices, while valine, isoleucine, phenylalanine, tyrosine, tryptophan and threonine are more commonly found in β-pleated sheets. Glycine, serine, aspartic acid, asparagine, and proline are commonly found in turns. Some preferred substitutions may be made among the following groups: (i) S and T; (ii) P and G; and (iii) A, V, L and I. Given the known genetic code, and recombinant and synthetic DNA techniques, the skilled scientist readily can construct DNAs encoding the conservative amino acid variants.

### Glycosylation Variants

Where the antibody comprises an Fe region, the carbohydrate attached thereto may be altered. Native antibodies produced by mammalian cells typically comprise a branched, biantennary oligosaccharide that is generally attached by an N-linkage to Asn297 using Kabat EU numbering of the CH2 domain of the Fc region; see, e.g., Wright et al. Trends Biotechnol. 15: 26-32 (1997).

In certain embodiments, an antibody provided herein is altered to increase or decrease the extent to which the antibody is glycosylated. Addition or deletion of glycosylation sites to an antibody may be conveniently accomplished by altering the expression system (e.g. host cell) and/or by altering the amino acid sequence such that one or more glycosylation sites is created or removed.

In one embodiment of this disclosure, aglycosyl antibodies having decreased effector function or antibody derivatives are prepared by expression in a prokaryotic host. Suitable prokaryotic hosts for include but are not limited to E. coli, Bacillus subtilis, Salmonella typhimurium and various species within the genera Pseudomonas, Streptomyces, and Staphylococcus.

In one embodiment, antibody variants are provided having decreased effector function, which are characterized by a modification at the conserved N-linked site in the CH2 domains of the Fc portion of said antibody. In one embodiment of present disclosure, the modification comprises a mutation at the heavy chain glycosylation site to prevent glycosylation at the site. Thus, in one preferred embodiment of this disclosure, the aglycosyl antibodies or antibody derivatives are prepared by mutation of the heavy chain glycosylation site, —i.e., mutation of N297 using Kabat EU numbering and expressed in an appropriate host cell.

In another embodiment of the present disclosure, aglycosyl antibodies or antibody derivatives have decreased effector function, wherein the modification at the conserved N-linked site in the CH2 domains of the Fc portion of said antibody or antibody derivative comprises the removal of the CH2 domain glycans, —i.e., deglycosylation. These aglycosyl antibodies may be generated by conventional methods and then deglycosylated enzymatically. Methods for enzymatic deglycosylation of antibodies are well known in the art (e.g. Winkelhake & Nicolson (1976), J Biol Chem. 251(4):1074-80).

In another embodiment of this disclosure, deglycosylation may be achieved using the glycosylation inhibitor tunicamycin (Nose & Wigzell (1983), Proc Natl Acad Sci USA, 80(21):6632-6). That is, the modification is the prevention of glycosylation at the conserved N-linked site in the CH2 domains of the Fc portion of said antibody.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function.

Examples of publications related to “defucosylated” or “fucose-deficient” antibody variants include: Okazaki et al. J Mol. Biol. 336: 1239-1249 (2004); Yamane-Ohnuki et al. Biotech. Bioeng. 87: 614 (2004).

Examples of cell lines capable of producing defucosylated antibodies include Lec13 CHO cells deficient in protein fucosylation (Ripka et al. Arch. Biochem. Biophys. 249:533-545 (1986); and WO 2004/056312), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006)).

Antibody variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878; U.S. Pat. No. 6,602,684; and US 2005/0123546.

Antibody variants with at least one galactose residue in the oligosaccharide attached to the Fc region are also provided. Such antibody variants may have improved CDC function. Such antibody variants are described, e.g., in WO1997/30087; WO1998/58964; and WO1999/22764.

### FC Region Variants

In certain embodiments, one or more amino acid modifications (e.g. a substitution) may be introduced into the Fc region of an antibody (e.g., a human IgG1, IgG2, IgG3 or IgG4 Fc region) provided herein, thereby generating an Fc region variant.

In certain embodiments, the disclosure contemplates an antibody variant that possesses some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity) but retains FcRn binding ability. In some embodiments, alterations are made in the Fc region that result in altered (i.e., either improved or diminished) C1q binding and/or Complement Dependent Cytotoxicity (CDC).

In certain embodiments, the disclosure contemplates an antibody variant that possesses an increased or decreased half-live. Antibodies with increased half-lives and improved binding to the neonatal Fe receptor (FcRn), which is responsible for the transfer of maternal IgGs to the fetus (Guyer et al., J Immunol. 117:587 (1976) and Kim et al., J Immunol. 24:249 (1994)), are described in US2005/0014934 (Hinton et al.). Those antibodies comprise an Fc region with one or more substitutions therein which improve binding of the Fc region to FcRn.

### Antibody Generation

An antibody of the disclosure may be derived from a recombinant antibody library that is based on amino acid sequences that have been isolated from the antibodies of a large number of healthy volunteers e.g. using the n-CoDeR® technology the fully human CDRs are recombined into new antibody molecules (Carlson & Söderlind, Expert Rev Mol Diagn. 2001 May; 1(1):102-8). Or alternatively for example antibody libraries as the fully human antibody phage display library described in Hoet R M et al., Nat Biotechnol 2005; 23(3):344-8) can be used to isolate Sema3A-specific antibodies. Antibodies or antibody fragments isolated from human antibody libraries are considered human antibodies or human antibody fragments herein.

Human antibodies may be further prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. For example immunization of genetically engineered mice inter alia immunization of hMAb mice (e.g. VelocImmune Mouse® or XENOMOUSE®) may be performed.

Further antibodies may be generated using the hybridoma technology (for example see Köhler and Milstein Nature. 1975 Aug. 7; 256(5517):495-7), resulting in for example murine, rat, or rabbit antibodies which can be converted into chimeric or humanized antibodies. Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Natl Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall' Acqua et al., Methods 36:43-60 (2005) (describing “FR shuffling”); and Osboum et al., Methods 36:61-68 (2005) and Klimka et al., Br. J. Cancer, 83:252-260 (2000) (describing the “guided selection” approach to FR shuffling).

Examples are provided for the generation of antibodies using a recombinant antibody library.

### DNA Molecules According to the Present Disclosure

The present disclosure also relates to an isolated nucleic acid sequence that encodes the antibody or antigen-binding fragment according to the present disclosure. The isolated nucleic acid sequence encoding the antibody or antigen-binding fragment according to the present disclosure can for instance be produced by techniques described in Sambrook et al., 1989, and Ausubel et al., 1989, or alternatively, by chemically synthesis. (e.g. techniques described in Oligonucleotide Synthesis (1984, Gait, ed., IRL Press, Oxford)). The DNA sequences and respective SEQ IDs used for the antibodies expressed are given in Table 1 and 1A. These sequences are optimized in certain cases for mammalian expression. DNA molecules of the disclosure are not limited to the sequences disclosed herein, but also include variants thereof. DNA variants within the disclosure may be described by reference to their physical properties in hybridization. The skilled worker will recognize that DNA can be used to identify its complement and, since DNA is double stranded, its equivalent or homolog, using nucleic acid hybridization techniques. It also will be recognized that hybridization can occur with less than 100% complementarity. However, given appropriate choice of conditions, hybridization techniques can be used to differentiate among DNA sequences based on their structural relatedness to a particular probe. For guidance regarding such conditions see, Sambrook et al., 1989 supra and Ausubel et al., 1995 (Ausubel, F. M., Brent, R., Kingston, R. E., Moore, D. D., Sedman, J. G., Smith, J. A., & Struhl, K. eds. (1995). Current Protocols in Molecular Biology. New York: John Wiley and Sons).

Structural similarity between two polynucleotide sequences can be expressed as a function of “stringency” of the conditions under which the two sequences will hybridize with one another. As used herein, the term “stringency” refers to the extent that the conditions disfavor hybridization. Stringent conditions strongly disfavor hybridization, and only the most structurally related molecules will hybridize to one another under such conditions. Conversely, non-stringent conditions favor hybridization of molecules displaying a lesser degree of structural relatedness. Hybridization stringency, therefore, directly correlates with the structural relationships of two nucleic acid sequences.

Hybridization stringency is a function of many factors, including overall DNA concentration, ionic strength, temperature, probe size and the presence of agents which disrupt hydrogen bonding. Factors promoting hybridization include high DNA concentrations, high ionic strengths, low temperatures, longer probe size and the absence of agents that disrupt hydrogen bonding. Hybridization typically is performed in two phases: the “binding” phase and the “washing” phase.

### Functionally Equivalent DNA Variants

Yet another class of DNA variants within the scope of the disclosure may be described with reference to the product they encode. These functionally equivalent polynucleotides are characterized by the fact that they encode the same peptide sequences due to the degeneracy of the genetic code.

It is recognized that variants of DNA molecules provided herein can be constructed in several different ways. For example, they may be constructed as completely synthetic DNAs. Methods of efficiently synthesizing oligonucleotides are widely available. See Ausubel et al., section 2.11, Supplement 21 (1993). Overlapping oligonucleotides may be synthesized and assembled in a fashion first reported by Khorana et al., J. Mol. Biol. 72:209-217 (1971); see also Ausubel et al., supra, Section 8.2. Synthetic DNAs preferably are designed with convenient restriction sites engineered at the 5′ and 3′ ends of the gene to facilitate cloning into an appropriate vector.

As indicated, a method of generating variants is to start with one of the DNAs disclosed herein and then to conduct site-directed mutagenesis. See Ausubel et al., supra, chapter 8, Supplement 37 (1997). In a typical method, a target DNA is cloned into a single-stranded DNA bacteriophage vehicle. Single-stranded DNA is isolated and hybridized with an oligonucleotide containing the desired nucleotide alteration(s). The complementary strand is synthesized and the double stranded phage is introduced into a host. Some of the resulting progeny will contain the desired mutant, which can be confirmed using DNA sequencing. In addition, various methods are available that increase the probability that the progeny phage will be the desired mutant. These methods are well known to those in the field and kits are commercially available for generating such mutants.

### Recombinant DNA Constructs and Expression

The present disclosure further provides recombinant DNA constructs comprising one or more of the nucleotide sequences according to the present disclosure. The recombinant constructs of the present disclosure can be used in connection with a vector, such as a plasmid, phagemid, phage or viral vector, into which a DNA molecule encoding an antibody of the disclosure or antigen-binding fragment thereof or variant thereof is inserted.

Thus, in one aspect, the present disclosure relates to a vector comprising a nucleic acid sequence according to the present disclosure.

An antibody, antigen binding portion, or variant thereof provided herein can be prepared by recombinant expression of nucleic acid sequences encoding light and heavy chains or portions thereof in a host cell. To express an antibody, antigen binding portion, or variant thereof recombinantly a host cell can be transfected with one or more recombinant expression vectors carrying DNA fragments encoding the light and/or heavy chains or portions thereof such that the light and heavy chains are expressed in the host cell. Standard recombinant DNA methodologies are used to prepare and/or obtain nucleic acids encoding the heavy and light chains, incorporate these nucleic acids into recombinant expression vectors and introduce the vectors into host cells, such as those described in Sambrook, Fritsch and Maniatis (eds.), Molecular Cloning; A Laboratory Manual, Second Edition, Cold Spring Harbor, N.Y., (1989), Ausubel, F. M. et al. (eds.) Current Protocols in Molecular Biology, Greene Publishing Associates, (1989) and in U.S. Pat. No. 4,816,397 by Boss et al.

In addition, the nucleic acid sequences encoding variable regions of the heavy and/or light chains can be converted, for example, to nucleic acid sequences encoding full-length antibody chains, Fab fragments, or to scFv. The VL- or VH-encoding DNA fragment can be operatively linked, (such that the amino acid sequences encoded by the two DNA fragments are in-frame) to another DNA fragment encoding, for example, an antibody constant region or a flexible linker. The sequences of human heavy chain and light chain constant regions are known in the art (see e.g., Kabat, E. A., el al. (1991) Sequences of Proteins of Immunological Interest, Fifth Edition, U.S. Department of Health and Human Services, NIH Publication No. 91-3242) and DNA fragments encompassing these regions can be obtained by standard PCR amplification.

To create a polynucleotide sequence that encodes a scFv, the VH- and VL-encoding nucleic acids can be operatively linked to another fragment encoding a flexible linker such that the VH and VL sequences can be expressed as a contiguous single-chain protein, with the VL and VH regions joined by the flexible linker (see e.g., Bird et al. (1988) Science 242:423-426; Huston et al. (1988) Proc. Natl. Acad. Sci. USA 85:5879-5883; McCafferty et al., Nature (1990) 348:552-554).

To express the antibodies, antigen binding fragments thereof or variants thereof standard recombinant DNA expression methods can be used (see, for example, Goeddel; Gene Expression Technology. Methods in Enzymology 185, Academic Press, San Diego, Calif. (1990)). For example, DNA encoding the desired polypeptide can be inserted into an expression vector which is then transfected into a suitable host cell. Suitable host cells are prokaryotic and eukaryotic cells. Examples for prokaryotic host cells are e.g. bacteria, examples for eukaryotic hosts cells are yeasts, insects and insect cells, plants and plant cells, transgenic animals, or mammalian cells. Introduction of the recombinant construct into the host cell can be carried out using standard techniques such as calcium phosphate transfection, DEAE dextran mediated transfection, electroporation, transduction or phage infection.

In some embodiments, the DNAs encoding the heavy and light chains are inserted into separate vectors. In other embodiments, the DNA encoding the heavy and light chains is inserted into the same vector. It is understood that the design of the expression vector, including the selection of regulatory sequences is affected by factors such as the choice of the host cell, the level of expression of protein desired and whether expression is constitutive or inducible.

Thus, in a further aspect, the present disclosure relates to an isolated cell expressing the antibody or antigen-binding fragment according to the present disclosure and/or comprising the nucleic acid according to the present disclosure or the vector according to the present disclosure.

The isolated cell can be virtually any cell for which expression vectors are available. The isolated cell can for example a higher eukaryotic host cell, such as a mammalian cell, a lower eukaryotic host cell, such as a yeast cell, and may be a prokaryotic cell, such as a bacterial cell.

In a further aspect, the present disclosure relates to a method of producing the isolated antibody or antigen-binding fragment according to the present disclosure comprising culturing of the cell according to the present disclosure. In particular embodiments, the cell according to the present disclosure is cultivated under suitable conditions for antibody expression and the antibody or antigen-binding fragment thereof is recovered. In particular embodiments, the antibody or antigen-binding fragment thereof is purified, particularly to at least 95% homogeneity by weight.

### Bacterial Expression

Useful expression vectors for bacterial use are constructed by inserting a DNA sequence encoding a desired protein together with suitable translation initiation and termination signals in operable reading phase with a functional promoter. The vector will comprise one or more phenotypic selectable markers and an origin of replication to ensure maintenance of the vector and, if desirable, to provide amplification within the host. Suitable prokaryotic hosts for transformation include but are not limited to E. coli, Bacillus subtilis, Salmonella typhimurium and various species within the genera Pseudomonas, Streptomyces, and Staphylococcus.

Bacterial vectors may be, for example, bacteriophage-, plasmid- or phagemid-based. These vectors can contain a selectable marker and a bacterial origin of replication derived from commercially available plasmids typically containing elements of the well-known cloning vector pBR322 (ATCC 37017). Following transformation of a suitable host strain and growth of the host strain to an appropriate cell density, the selected promoter is de-repressed/induced by appropriate means (e.g., temperature shift or chemical induction) and cells are cultured for an additional period. Cells are typically harvested by centrifugation, disrupted by physical or chemical means, and the resulting crude extract retained for further purification.

In bacterial systems, a number of expression vectors may be advantageously selected depending upon the use intended for the protein being expressed. For example, when a large quantity of such a protein is to be produced, for the generation of antibodies or to screen peptide libraries, for example, vectors which direct the expression of high levels of fusion protein products that are readily purified may be desirable.

Therefore, an embodiment of the present disclosure is an expression vector comprising a nucleic acid sequence encoding for the novel antibodies of the present disclosure.

Antibodies of the present disclosure or antigen-binding fragments thereof or variants thereof include naturally purified products, products of chemical synthetic procedures, and products produced by recombinant techniques from a prokaryotic host, including, for example, E. coli, Bacillus subtilis, Salmonella typhimurium and various species within the genera Pseudomonas, Streptomyces, and Staphylococcus, preferably, from E. coli cells.

### Mammalian Expression

Preferred regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma. Expression of the antibodies may be constitutive or regulated (e.g. inducible by addition or removal of small molecule inductors such as Tetracyclin in conjunction with Tet system). For further description of viral regulatory elements, and sequences thereof, see e.g., U.S. Pat. No. 5,168,062 by Stinski, U.S. Pat. No. 4,510,245 by Bell et al. and U.S. Pat. No. 4,968,615 by Schaffner et al. The recombinant expression vectors can also include origins of replication and selectable markers (see e.g., U.S. Pat. Nos. 4,399,216, 4,634,665 and 5,179,017). Suitable selectable markers include genes that confer resistance to drugs such as G418, puromycin, hygromycin, blasticidin, zeocin/bleomycin or methotrexate or selectable marker that exploit auxotrophies such as Glutamine Synthetase (Bebbington et al., Biotechnology (N Y). 1992 February; 10(2):169-75), on a host cell into which the vector has been introduced. For example, the dihydrofolate reductase (DHFR) gene confers resistance to methotrexate, neo gene confers resistance to G418, the bsd gene from Aspergillus terreus confers resistance to blasticidin, puromycin N-acetyl-transferase confers resistance to puromycin, the Sh ble gene product confers resitance to zeocin, and resistance to hygromycin is conferred by the E. coli hygromycin resistance gene (hyg or hph). Selectable markers like DHFR or Glutamine Synthetase are also useful for amplification techniques in conjunction with MTX and MSX.

Transfection of the expression vector into a host cell can be carried out using standard techniques such as electroporation, nucleofection, calcium-phosphate precipitation, lipofection, polycation-based transfection such as polyethlylenimine (PEI)-based transfection and DEAE-dextran transfection.

Suitable mammalian host cells for expressing the antibodies, antigen binding fragments thereof or variants thereof provided herein include Chinese Hamster Ovary (CHO cells) such as CHO-K1, CHO-S, CHO-K1SV [including dhfr− CHO cells, described in Urlaub and Chasin, (1980) Proc. Natl. Acad. Sci. USA 77:4216-4220 and Urlaub et al., Cell. 1983 June; 33(2):405-12, used with a DHFR selectable marker, e.g., as described in R. J. Kaufman and P. A. Sharp (1982) Mol. Biol. 159:601-621; and other knockout cells exemplified in Fan et al., Biotechnol Bioeng. 2012 April; 109(4):1007-15], NSO myeloma cells, COS cells, HEK293 cells, HKB11 cells, BHK21 cells, CAP cells, EB66 cells, and SP2 cells.

Expression might also be transient or semi-stable in expression systems such as HEK293, HEK293T, HEK293-EBNA, HEK293E, HEK293-6E, HEK293-Freestyle, HKB11, Expi293F, 293EBNALT75, CHO Freestyle, CHO-S, CHO-K1, CHO-K1SV, CHOEBNALT85, CHOS-XE, CHO-3E7 or CAP-T cells (for instance Durocher et al., Nucleic Acids Res. 2002 Jan. 15; 30(2):E9).

In some embodiments, the expression vector is designed such that the expressed protein is secreted into the culture medium in which the host cells are grown. The antibodies, antigen binding fragments thereof or variants thereof can be recovered from the culture medium using standard protein purification methods.

### Purification

Antibodies of the disclosure or antigen-binding fragments thereof or variants thereof can be recovered and purified from recombinant cell cultures by well-known methods including, but not limited to ammonium sulfate or ethanol precipitation, acid extraction, Protein A chromatography, Protein G chromatography, anion or cation exchange chromatography, phospho-cellulose chromatography, hydrophobic interaction chromatography, affinity chromatography, hydroxylapatite chromatography and lectin chromatography. High performance liquid chromatography (“HPLC”) can also be employed for purification. See, e.g., Colligan, Current Protocols in Immunology, or Current Protocols in Protein Science, John Wiley & Sons, NY, N.Y., (1997-2001), e.g., Chapters 1, 4, 6, 8, 9, 10, each entirely incorporated herein by reference.

Antibodies of the present disclosure or antigen-binding fragments thereof or variants thereof include naturally purified products, products of chemical synthetic procedures, and products produced by recombinant techniques from a eukaryotic host, including, for example, yeast, higher plant, insect and mammalian cells. Depending upon the host employed in a recombinant production procedure, the antibody of the present disclosure can be glycosylated or can be non-glycosylated. Such methods are described in many standard laboratory manuals, such as Sambrook, supra, Sections 17.37-17.42; Ausubel, supra, Chapters 10, 12, 13, 16, 18 and 20.

In preferred embodiments, the antibody is purified (1) to greater than 95% by weight of antibody as determined e.g. by the Lowry method, UV-Vis spectroscopy or by by SDS-Capillary Gel electrophoresis (for example on a Caliper LabChip GXII, GX 90 or Biorad Bioanalyzer device), and in further preferred embodiments more than 99% by weight, (2) to a degree sufficient to obtain at least 15 residues of N-terminal or internal amino acid sequence, or (3) to homogeneity by SDS-PAGE under reducing or non-reducing conditions using Coomassie blue or, preferably, silver stain. Isolated naturally occurring antibody includes the antibody in situ within recombinant cells since at least one component of the antibody's natural environment will not be present. Ordinarily, however, isolated antibody will be prepared by at least one purification step.

### Therapeutic Methods

Therapeutic methods involve administering to a subject in need of treatment a therapeutically effective amount of an antibody or an antigen-binding fragment thereof or a variant thereof contemplated by the disclosure. A “therapeutically effective” amount hereby is defined as the amount of an antibody or antigen-binding fragment thereof that is of sufficient quantity to decrease Sema3A activity in a subject—either as a single dose or according to a multiple dose regimen, alone or in combination with other agents, which leads to the alleviation of an adverse condition, yet which amount is toxicologically tolerable. The subject may be a human or non-human animal (e.g., rabbit, rat, mouse, dog, monkey or other lower-order primate).

Thus, in one aspect, the present disclosure relates to the isolated antibody or antigen-binding fragment according the present disclosure or to a conjugate comprising the isolated antibody or antigen-binding fragment according the present disclosure or to a pharmaceutical composition comprising the isolated antibody or antigen-binding fragment according the present disclosure for use as a medicament.

The isolated antibody or antigen-binding fragment according to the present disclosure can be used as a therapeutic or a diagnostic tool in a variety of Sema3A-associated disorders.

Thus, in a further aspect, the present disclosure relates to the isolated antibody or antigen-binding fragment according the present disclosure or to a conjugate comprising the isolated antibody or antigen-binding fragment according the present disclosure or to a pharmaceutical composition comprising the isolated antibody or antigen-binding fragment according the present disclosure for use in the treatment and/or prevention of renal diseases, in particular of acute and chronic kidney diseases, diabetic kidney diseases, Alport syndrome and of acute and chronic renal failure. The general terms ‘renal disease’ or ‘kidney disease’ describes a class of conditions in which the kidneys fail to filter and remove waste products from the blood. There are two major forms of kidney disease: acute kidney disease (acute kidney injury, AKI) and chronic kidney disease (CKD). The isolated antibody or antigen-binding fragment according to the present disclosure or a conjugate or pharmaceutical composition comprising the same may further be used for the treatment and/or prevention of sequelae of acute kidney injury arising from multiple insults such as ischemia-reperfusion injury, radiocontrast administration, cardiopulmonary bypass surgery, shock and sepsis. In the context of the present disclosure, the terms renal failure and renal insufficiency comprise both acute and chronic manifestations of renal insufficiency, as well as underlying or related kidney diseases such as renal hypoperfusion, intradialytic hypotension, obstructive uropathy, glomerulopathies, IgA nephropathy, glomerulonephritis, acute glomerulonephritis, glomerulosclerosis, tubulointerstitial diseases, nephropathic diseases such as primary and congenital kidney disease, nephritis, Alport syndrome, kidney inflammation, immunological kidney diseases such as kidney transplant rejection, immune complex-induced kidney diseases, nephropathy induced by toxic substances, contrast medium-induced nephropathy; minimal change glomerulonephritis (lipoid); Membranous glomerulonephritis; focal segmental glomerulosclerosis (FSGS); hemolytic uremic syndrome (HUS), amyloidosis, Goodpasture's syndrome, Wegener's granulomatosis, Purpura Schönlein-Henoch, diabetic and non-diabetic nephropathy, pyelonephritis, renal cysts, nephrosclerosis, hypertensive nephrosclerosis and nephrotic syndrome, which can be characterized diagnostically, for example, by abnormally reduced creatinine and/or water excretion, abnormally increased blood concentrations of urea, nitrogen, potassium and/or creatinine, altered activity of renal enzymes such as, for example, glutamyl synthetase, altered urine osmolarity or urine volume, increased microalbuminuria, macroalbuminuria, lesions of glomeruli and arterioles, tubular dilatation, hyperphosphatemia and/or the need for dialysis. The present disclosure also relates to the isolated antibody or antigen-binding fragment according to the present disclosure or a conjugate or pharmaceutical composition comprising same for use in the treatment and/or prevention of sequelae of renal insufficiency, for example pulmonary edema, heart failure, uremia, anemia, electrolyte disturbances (e.g. hyperkaliemia, hyponatremia) and disturbances in bone and carbohydrate metabolism. The compounds according to the disclosure are also suitable for the treatment and/or prevention of polycystic kidney disease (PCKD) and of the syndrome of inadequate ADH secretion (SIADH).

Additionally, the isolated antibody or antigen-binding fragment according to the present disclosure or a conjugate or pharmaceutical composition comprising the same may be used for the treatment and/or prevention of vascular hyperpermeability, diabetic retinopathy, deterioration of the blood retinal barrier and consequent macular edema, preferably, age related macular edema, non-proliferative age-related macular edema and non-proliferative diabetic macular edema.

Further, the isolated antibody or antigen-binding fragment according to the present disclosure or a conjugate or pharmaceutical composition comprising same is suitable for the prevention or treatment of disease of the central or peripheral nervous system like neuropathic pain, spinal cord injury, multiple sclerosis, traumatic brain injury, brain edema or neurodegenerative diseases in which the neurodegenerative disease is Alzheimer's disease, Parkinson's disease, Huntington's disease, amyotrophic lateral sclerosis, progressive supranuclear paralysis, black substance degeneration, Shy-Drager syndrome, olivopontocerebellar atrophy or spinocerebellar degeneration.

Furthermore, the isolated antibody or antigen-binding fragment according to the present disclosure or a conjugate or pharmaceutical composition comprising the same may be useful for the treatment and/or prevention of cancer, wherein the cancer is intestinal cancer, colorectal cancer, lung cancer, breast cancer, brain cancer, melanoma, renal cell cancer, leukemia, lymphoma, T-cell lymphoma, stomach cancer, pancreatic cancer, cervical cancer, endometrial cancer, ovarian cancer, esophageal cancer, liver cancer, squamous cell carcinoma of the head and neck, skin cancer, urinary tract cancer, prostate cancer, choriocarcinoma, pharyngeal cancer or larynx cancer.

The disorders mentioned above have been well characterized in humans, but also exist with a similar etiology in other animals, including mammals, and can be treated by administering pharmaceutical compositions according to the present disclosure.

The antibody or the antigen-binding fragment according to the present disclosure or a variant thereof might be co-administered with known medicaments, and in some instances the antibody or antigen-binding fragment thereof might itself be modified. For example, an antibody or an antigen-binding fragment thereof or a variant thereof could be conjugated to a drug or to another peptide or protein to potentially further increase efficacy.

Antibodies of the present disclosure or antigen-binding fragments thereof or variants thereof may be administered as the sole pharmaceutical agent or in combination with one or more additional therapeutic agents where the combination causes no unacceptable adverse effects.

Thus, in a further aspect, the present disclosure relates to the isolated antibody or antigen-binding fragment according to the present disclosure or the conjugate according to the present disclosure or the pharmaceutical composition according to the present disclosure for use in simultaneous, separate, or sequential combination with one or more further therapeutically active compounds.

Non-limiting examples of therapeutically active compounds to be used in combination with the antibody or antigen-binding fragment according to the present disclosure are:
blood pressure lowering agents, for example and preferably from the group of calcium antagonists, angiotensin II antagonists, ACE inhibitors, NEP inhibitors, vasopeptidase inhibitors, endothelin antagonists, renin inhibitors, alpha-blockers, beta-blockers, mineralocorticoid receptor antagonists and diuretics;
antidiabetic agents (hypoglycemic or antihyperglycemic agents), such as for example and preferably insulin and derivatives, sulfonylureas, biguanides, thiazolidinediones, acarbose, DPP4 inhibitors, GLP-1 analogues, or SGLT inhibitors (gliflozins);
compounds inhibiting the signal transduction cascade, in particular tyrosine and/or serine/threonine kinase inhibitors, such as for example nintedanib, dasatinib, nilotinib, bosutinib, regorafenib, sorafenib, sunitinib, cediranib, axitinib, telatinib, imatinib, brivanib, pazopanib, vatalanib, gefitinib, erlotinib, lapatinib, canertinib, lestaurtinib, pelitinib, semaxanib or tandutinib;
anti-inflammatory drugs such as non-steroidal anti-inflammatory drugs (NSAIDs) including acetylsalicylic acid (aspirin), ibuprofen and naproxen, glucocorticoids such as for example and preferably prednison, prednisolon, methylprednisolon, triamcinolon, dexamethason, beclomethason, betamethason, flunisolid, budesonid or fluticason, or 5-aminosalicylic acid derivatives, leukotriene antagonists, TNF-alpha inhibitors and chemokine receptor antagonists such as CCR1, 2 and/or 5 inhibitors, NF-κB inhibitors and Nerf2 activators;
anti-fibrotic drugs such as TGFbeta antagonist, or microRNA-21 inhibitors;
organic nitrates and NO-donors, for example sodium nitroprusside, nitroglycerin, isosorbide mononitrate, isosorbide dinitrate, molsidomine or SIN-1, and inhalational NO;
compounds that inhibit the degradation of cyclic guanosine monophosphate (cGMP), for example inhibitors of phosphodiesterases (PDE) 1, 2, 5 and/or 9, in particular PDE-5 inhibitors such as sildenafil, vardenafil, tadalafil, udenafil, dasantafil, avanafil, mirodenafil, lodenafil, CTP-499 or PF-00489791;
calcium sensitizers, such as for example and preferably levosimendan;
antithrombotic agents, particularly selected from the group consisting of platelet aggregation inhibitors, anticoagulants and profibrinolytic substances;
agents, that stimulate NO- and heme-dependent as well as NO- and heme-indipendent the synthesis of cGMP, for example and with preference soluble guanylate cyclase (sGC) modulators, for example and with preference riociguat, cinaciguat, vericiguat or BAY 1101042;
fat metabolism altering agents, for example and preferably from the group of thyroid receptor agonists, cholesterol synthesis inhibitors, such as for example and preferably HMG-CoA-reductase or squalene synthesis inhibitors, ACAT inhibitors, CETP inhibitors, MTP inhibitors, PPAR-alpha, PPAR-gamma and/or PPAR-delta agonists, cholesterol absorption inhibitors, lipase inhibitors, polymeric bile acid adsorbers, bile acid reabsorption inhibitors and lipoprotein(a) antagonists.

In particular embodiments, the isolated antibody or antigen-binding fragment according to the present disclosure is administered in combination with a platelet aggregation inhibitor, particularly aspirin, clopidogrel, ticlopidine or dipyridamole.

In particular embodiments, the isolated antibody or antigen-binding fragment according to the present disclosure is administered in combination with a thrombin inhibitor, particularly ximelagatran, dabigatran, melagatran, bivalirudin or enoxaparin.

In particular embodiments, the isolated antibody or antigen-binding fragment according to the present disclosure is administered in combination with a GPIIb/IIIa antagonist, particularly tirofiban or abciximab.

In particular embodiments, the isolated antibody or antigen-binding fragment according to the present disclosure is administered in combination with a factor Xa inhibitor, particularly selected from rivaroxaban, apixaban, otamixaban, fidexaban, razaxaban, fondaparinux, idraparinux, DU-176b, PMD-3112, YM-150, KFA-1982, EMD-503982, MCM-17, MLN-1021, DX 9065a, DPC 906, JTV 803, SSR-126512 and SSR-128428.

In particular embodiments, the isolated antibody or antigen-binding fragment according to the present disclosure is administered in combination with heparin or a low molecular weight (LMW) heparin derivative.

In particular embodiments, the isolated antibody or antigen-binding fragment according to the present disclosure is administered in combination with a vitamin K antagonist, particularly selected from coumarin.

Blood pressure lowering agents are particularly selected from the group consisting of calcium antagonists, angiotensin AII antagonists, ACE inhibitors, NEP inhibitors, vasopeptidase inhibitors, endothelin antagonists, renin inhibitors, alpha-blockers, beta-blockers, mineralocorticoid receptor antagonists and diuretics.

In particular embodiments, the isolated antibody or antigen-binding fragment according to the present disclosure is administered in combination with a calcium antagonist, particularly selected from nifedipine, amlodipine, verapamil and diltiazem.

In particular embodiments, the isolated antibody or antigen-binding fragment according to the present disclosure is administered in combination with an angiotensin AII receptor antagonist, particularly selected from the group consisting of losartan, candesartan, valsartan, telmisartan, irbesartan, olmesartan, eprosartan, embursartan and azilsartan.

In particular embodiments, the isolated antibody or antigen-binding fragment according to the present disclosure is administered in combination with an ACE inhibitor, particularly selected from the group consisting of enalapril, captopril, lisinopril, ramipril, delapril, fosinopril, quinopril, perindopril, benazepril and trandopril.

In particular embodiments, the isolated antibody or antigen-binding fragment according to the present disclosure is administered in combination with an endothelin antagonist, particularly selected from the group consisting of bosentan, darusentan, ambrisentan, tezosentan, sitaxsentan, avosentan, macitentan and atrasentan.

In particular embodiments, the isolated antibody or antigen-binding fragment according to the present disclosure is administered in combination with a renin inhibitor, particularly selected from the group consisting of aliskiren, SPP-600 and SPP-800.

In particular embodiments, the isolated antibody or antigen-binding fragment according to the present disclosure is administered in combination with a mineralocorticoid receptor antagonist, particularly selected from the group consisting of finerenone, spironolactone, canrenone, potassium canrenoate, eplerenone, esaxerenone (CS-3150), or apararenone (MT-3995), CS-3150, and MT-3995.

In particular embodiments, the isolated antibody or antigen-binding fragment according to the present disclosure is administered in combination with a diuretic, particularly selected from the group consisting of furosemide, bumetanide, piretanide, torsemide, bendroflumethiazide, chlorothiazide, hydrochlorothiazide, xipamide, indapamide, hydroflumethiazide, methyclothiazide, polythiazide, trichloromethiazide, chlorothalidone, metolazone, quinethazone, acetazolamide, dichlorophenamide, methazolamide, glycerine, isosorbide, mannitol, amiloride and triamterene.

Fat metabolism altering agents are particularly selected from the group consisting of CETP inhibitors, thyroid receptor agonists, cholesterol synthesis inhibitors such as HMG-CoA-reductase or squalene synthesis inhibitors, ACAT inhibitors, MTP inhibitors, PPAR-alpha, PPAR-gamma and/or PPAR-delta agonists, cholesterol absorption inhibitors, polymeric bile acid adsorbers, bile acid reabsorption inhibitors, lipase inhibitors and lipoprotein(a) antagonists.

In particular embodiments, the isolated antibody or antigen-binding fragment according to the present disclosure is administered in combination with a Nerf2 activator, particularly selected from Bardoxolone methyl.

In particular embodiments, the isolated antibody or antigen-binding fragment according to the present disclosure is administered in combination with a thyroid receptor agonist, particularly selected from the group consisting of D-thyroxin, 3,5,3′-triiodothyronin (T3), CGS 23425 and axitirome (CGS 26214).

In particular embodiments, the isolated antibody or antigen-binding fragment according to the present disclosure is administered in combination with an HMG-CoA-reductase inhibitor from the class of statins, particularly selected from the group consisting of lovastatin, simvastatin, pravastatin, fluvastatin, atorvastatin, rosuvastatin and pitavastatin.

In particular embodiments, the isolated antibody or antigen-binding fragment according to the present disclosure is administered in combination with a PPAR-gamma modulator, particularly selected from pioglitazone and rosiglitazone.

In particular embodiments, the isolated antibody or antigen-binding fragment according to the present disclosure is administered in combination with a PPAR-delta modulator, particularly selected from the group consisting of ASP1128, GW 501516 and BAY 68-5042.

In particular embodiments, the isolated antibody or antigen-binding fragment according to the present disclosure is administered in combination with a cholesterol absorption inhibitor, particularly selected from the group consisting of ezetimibe, tiqueside and pamaqueside.

In particular embodiments, the isolated antibody or antigen-binding fragment according to the present disclosure is administered in combination with a lipase inhibitor, particularly selected from orlistat.

In particular embodiments, the isolated antibody or antigen-binding fragment according to the present disclosure is administered in combination with a polymeric bile acid adsorber, particularly selected from the group consisting of cholestyramine, colestipol, colesolvam, CholestaGel and colestimide.

In particular embodiments, the isolated antibody or antigen-binding fragment according to the present disclosure is administered in combination with a bile acid reabsorption inhibitor, particularly selected from the group consisting of ASBT (IBAT) inhibitors such as AZD-7806, 5-8921, AK-105, BARI-1741, SC-435 and SC-635.

In particular embodiments, the isolated antibody or antigen-binding fragment according to the present disclosure is administered in combination with a lipoprotein(a) antagonist, particularly selected from the group consisting of gemcabene calcium (CI-1027) and nicotinic acid.

In particular embodiments, the isolated antibody or antigen-binding fragment according to the present disclosure is administered in combination with a TGFbeta antagonist, particularly selected from pirfenidone and fresolimumab.

In particular embodiments, the isolated antibody or antigen-binding fragment according to the present disclosure is administered in combination with anti-microRNA-21 oligonucleotides, particularly selected from Lademirsen.

In particular embodiments, the isolated antibody or antigen-binding fragment according to the present disclosure is administered in combination with HIF-PH inhibitors, particularly selected from molidustat and roxadustat.

In particular embodiments, the isolated antibody or antigen-binding fragment according to the present disclosure is administered in combination with a CCR2 antagonist, particularly selected from CCX-140.

In particular embodiments, the isolated antibody or antigen-binding fragment according to the present disclosure is administered in combination with a TNFalpha antagonist, particularly selected from adalimumab.

In particular embodiments, the isolated antibody or antigen-binding fragment according to the present disclosure is administered in combination with a galectin-3 inhibitor, particularly selected from GCS-100.

In particular embodiments, the isolated antibody or antigen-binding fragment according to the present disclosure is administered in combination with a hepatocyte growth factor mimeticmimetics, particularly selected from Refanalin.

In particular embodiments, the isolated antibody or antigen-binding fragment according to the present disclosure is administered in combination with a p53 modulator, particularly selected from QPI-1002.

In particular embodiments, the isolated antibody or antigen-binding fragment according to the present disclosure is administered in combination with a NOX1/4 inhibitor, particularly selected from GKT-137831.

In particular embodiments, the isolated antibody or antigen-binding fragment according to the present disclosure is administered in combination with a medicament which affects the vitamin D metabolism, particularly selected from cholecalciferol and paracalcitol.

In particular embodiments, the isolated antibody or antigen-binding fragment according to the present disclosure is administered in combination with a cytostatic agent, particularly selected from cyclophosphamide.

In particular embodiments, the isolated antibody or antigen-binding fragment according to the present disclosure is administered in combination with anti-VEGF therapy, particularly selected from the group consisting of ranibizumab, bevacizumab and aflibercept.

In particular embodiments, the isolated antibody or antigen-binding fragment according to the present disclosure is administered in combination with an immunosuppressive agent, particularly selected from ciclosporin.

In particular embodiments, the isolated antibody or antigen-binding fragment according to the present disclosure is administered in combination with a phosphate binder, particularly selected from sevelamer and lanthanum carbonate.

In particular embodiments, the isolated antibody or antigen-binding fragment according to the present disclosure is administered in combination with a calcimimetic for therapy of hyperparathyroidism.

In particular embodiments, the isolated antibody or antigen-binding fragment according to the present disclosure is administered in combination with agents for iron deficit therapy, particularly selected from iron products.

In particular embodiments, the isolated antibody or antigen-binding fragment according to the present disclosure is administered in combination with agents for the therapy of hyperurikaemia, particularly selected from allopurinol and rasburicase.

In particular embodiments, the isolated antibody or antigen-binding fragment according to the present disclosure is administered in combination with glycoprotein hormone for the therapy of anaemia, particularly selected from erythropoietin.

In particular embodiments, the isolated antibody or antigen-binding fragment according to the present disclosure is administered in combination with biologics for immune therapy, particularly selected from the group consisting of abatacept, rituximab, eculizumab and belimumab.

In particular embodiments, the isolated antibody or antigen-binding fragment according to the present disclosure is administered in combination with Jak inhibitors, particularly selected from the group consisting of ruxolitinib, tofacitinib, baricitinib, CYT387, GSK2586184, lestaurtinib, pacritinib (SB1518) and TG101348.

In particular embodiments, the isolated antibody or antigen-binding fragment according to the present disclosure is administered in combination with prostacyclin analogs for therapy of microthrombi.

In particular embodiments, the isolated antibody or antigen-binding fragment according to the present disclosure is administered in combination with an alkali therapy, particularly selected from sodium bicarbonate.

In particular embodiments, the isolated antibody or antigen-binding fragment according to the present disclosure is administered in combination with an mTOR inhibitor, particularly selected from everolimus and rapamycin.

In particular embodiments, the isolated antibody or antigen-binding fragment according to the present disclosure is administered in combination with an NHE3 inhibitor, particularly selected from AZD1722.

In particular embodiments, the isolated antibody or antigen-binding fragment according to the present disclosure is administered in combination with an eNOS modulator, particularly selected from sapropterin.

In particular embodiments, the isolated antibody or antigen-binding fragment according to the present disclosure is administered in combination with a CTGF inhibitor, particularly selected from FG-3019.

In particular embodiments, the isolated antibody or antigen-binding fragment according to the present disclosure is administered in combination with one or more additional therapeutic agents selected from the group consisting of diuretics, angiotensin AII antagonists, ACE inhibitors, beta-receptor blockers, mineralocorticoid receptor antagonists, antidiabetics, organic nitrates and NO donors, activators and stimulators of the soluble guanylate cyclase (sGC), and positive-inotropic agents.

In particular embodiments, the isolated antibody or antigen-binding fragment according to the present disclosure is administered in combination with one or more additional therapeutic agents selected from the group consisting of diuretics, angiotensin AII antagonists, ACE inhibitors, beta-receptor blockers, mineralocorticoid receptor antagonists, antidiabetics, organic nitrates and NO donors, activators and stimulators of the soluble guanylate cyclase (sGC), positive-inotropic agents, anti-inflammatory agents, immunosuppressive agents, phosphate binders and antibodies which modulate vitamin D metabolism.

Combination therapy includes administration of a single pharmaceutical dosage formulation which comprises the antibody or antigen-binding fragment according to the present disclosure or a variant thereof and one or more additional therapeutic agents, as well as administration of the antibody or antigen-binding fragment according to the present disclosure and each additional therapeutic agent in its own separate pharmaceutical dosage formulation. For example, an antibody of the disclosure or an antigen-binding fragment thereof or a variant thereof and a therapeutic agent may be administered to the patient together in a single liquid composition, or each agent may be administered in separate dosage formulation.

Where separate dosage formulations are used, the antibody or antigen-binding fragment according to the present disclosure or the variant thereof and one or more additional therapeutic agents may be administered at essentially the same time (e.g., concurrently) or at separately staggered times (e.g., sequentially).

The antibody or the antigen-binding fragment according to the present disclosure or a variant thereof might be used in combination with surgical interventions, including but not limited to:
major cardiovascular surgeries e.g. coronary artery bypass grafting (CABG), heart valve repair or replacement, insertion of a pacemaker or an implantable cardioverter defibrillator (ICD), maze surgery, aneurysm repair, arotid artery surgery/endarterectomy and thrombectomy;
major non-cardiac surgeries e.g., thoracic, orthopedic urologic surgeries.

### Diagnostic Methods

Furthermore, the antibody or antigen-binding fragment according to the present disclosure may be utilized, as such or in compositions, in research and diagnostics, or as analytical reference standards, and the like.

Anti-Sema3A antibodies or antigen-binding fragments thereof can be used for detecting the presence of Sema3A. Thus, in a further aspect, the present disclosure relates to the isolated antibody or antigen-binding fragment according to the present disclosure or the antibody conjugate according to the present disclosure for use as a diagnostic agent.

### Pharmaceutical Compositions and Administration

In a further aspect, the present disclosure relates to a pharmaceutical composition comprising the isolated antibody or antigen-binding fragment according to the present disclosure or the antibody conjugate according to the present disclosure. To treat any of the foregoing disorders, pharmaceutical compositions for use in accordance with the present disclosure may be formulated in any conventional manner using one or more physiologically acceptable carriers, excipients, or auxiliaries. Further details on techniques for formulation and administration may be found in the latest edition of Remington's Pharmaceutical Sciences (Ed. Maack Publishing Co, Easton, Pa.).

The antibody or antigen-binding fragment according to the present disclosure can be administered by any suitable means, which can vary, depending on the type of disorder being treated. Possible administration routes include oral, parenteral, and topical administration. Methods of parenteral delivery include intra-arterial, intramuscular, subcutaneous, intramedullary, intrathecal, intraventricular, intravenous, intraperitoneal, or intranasal administration. In addition, the antibody or antigen-binding fragment according to the present disclosure may be administered by pulse infusion, with, e.g., declining doses of the antibody. Preferably, administration is by injections, most preferably intravenous or subcutaneous injections, depending in part on whether the administration is brief or prolonged. The amount to be administered will depend on a variety of factors such as the clinical symptoms, weight of the individual, whether other drugs are administered, and the like. The skilled artisan will recognize that the route of administration will vary depending on the disorder or condition to be treated.

The pharmaceutical composition according to the present disclosure comprises the antibody or antigen-binding fragment according to the present disclosure alone or in combination with at least one other agent, such as a stabilizing compound. The antibody or antigen-binding fragment according to the present disclosure may be administered in any sterile, biocompatible pharmaceutical carrier, including, but not limited to, saline, buffered saline, dextrose, and water. In particular embodiments, the pharmaceutical composition according to the present disclosure may comprise one or more further pharmaceutically active compounds, in particular one or more further pharmaceutically active compounds that are suitable to treat Sema3A associated disorders. Any of these agents can be administered to a patient alone, or in combination with other agents or drugs, in pharmaceutical compositions where it is mixed with excipient(s) or pharmaceutically acceptable carriers. In particular embodiments, the pharmaceutically acceptable carrier is pharmaceutically inert.

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions and the like, for ingestion by the patient.

Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers such as sugars, including lactose, sucrose, mannitol, or sorbitol; starch from corn, wheat, rice, potato, or other plants; cellulose such as methyl-cellulose, hydroxypropylmethylcellulose, or sodium carboxymethyl cellulose; and gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores can be provided with suitable coatings such as concentrated sugar solutions, which may also contain gum arabic, talc, polyvinyl pyrrolidone, carbopol gel, polyethylene glycol and/or titanium dioxide, lacquer solutions, and suitable organic solvents or solvent mixtures. Dyestuffs or pigments may be added to the tablets or dragee coatings for product identification or to characterize the quantity of active compound, i.e. dosage.

Pharmaceutical preparations that can be used orally include push-fit capsules made of gelatin, as well as soft, sealed capsules made of gelatin and a coating such as glycerol or sorbitol. Push-fit capsules can contain active ingredients mixed with a filler or binders such as lactose or starches, lubricants such as talc or magnesium stearate, and optionally, stabilizers. In soft capsules, the active compounds may be dissolved or suspended in suitable liquids, such as fatty oils, liquid paraffin, or liquid polyethylene glycol with or without stabilizers.

Pharmaceutical formulations for parenteral administration include aqueous solutions of active compounds. For injection, the pharmaceutical compositions of the disclosure may be formulated in aqueous solutions, preferably in physiologically compatible buffers such as Hank's solution, Ringer's solution, or physiologically buffered saline. Aqueous injection suspensions may contain substances that increase viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions.

For topical or nasal administration, penetrants appropriate to the particular barrier to be permeated are used in the formulation. Such penetrants are generally known in the art.

The pharmaceutical compositions of the present disclosure may be manufactured in a manner that is known in the art, e.g., by means of conventional mixing, dissolving, granulating, dragee-making, levigating, emulsifying, encapsulating, entrapping or lyophilizing processes.

The pharmaceutical composition may be provided as a salt and can be formed with acids, including but not limited to hydrochloric, sulfuric, acetic, lactic, tartaric, malic, succinic, etc. Salts tend to be more soluble in aqueous or other protonic solvents that are the corresponding free base forms. In other cases, the preferred preparation may be a lyophilized powder in 1 mM-50 mM histidine or phosphate or Tris, 0.1%-2% sucrose and/or 2%-7% mannitol at a pH range of 4.5 to 7.5 optionally comprising additional substances like polysorbate that is combined with buffer prior to use.

After pharmaceutical compositions comprising a compound of the disclosure formulated in an acceptable carrier have been prepared, they can be placed in an appropriate container and labeled for treatment of an indicated condition. For administration of anti-Sema3A antibodies or antigen-binding fragment thereof, such labeling would include amount, frequency and method of administration.

### Therapeutically Effective Dose

The determination of an effective dose is well within the capability of those skilled in the art. Determining a therapeutically effective amount of the novel antibody of this disclosure or an antigen-binding fragment thereof or a variant thereof, largely will depend on particular patient characteristics, route of administration, and the nature of the disorder being treated. General guidance can be found, for example, in the publications of the International Conference on Harmonization and in REMINGTON'S PHARMACEUTICAL SCIENCES, chapters 27 and 28, pp. 484-528 (18th ed., Alfonso R. Gennaro, Ed., Easton, Pa.: Mack Pub. Co., 1990). More specifically, determining a therapeutically effective amount will depend on such factors as toxicity and efficacy of the medicament. Toxicity may be determined using methods well known in the art and found in the foregoing references. Efficacy may be determined utilizing the same guidance in conjunction with the methods described below in the Examples.

For any compound, the therapeutically effective dose can be estimated initially either in cell culture assays, or in animal models, usually mice, rabbits, dogs, pigs or monkeys. The animal model is also used to achieve a desirable concentration range and route of administration. Such information can then be used to determine useful doses and routes for administration in humans.

A therapeutically effective dose refers to that amount of antibody or antigen-binding fragment thereof, that ameliorates the symptoms or condition. Therapeutic efficacy and toxicity of such compounds can be determined by standard pharmaceutical procedures in cell cultures or experimental animals, e.g., ED₅₀ (the dose therapeutically effective in 50% of the population) and LD₅₀ (the dose lethal to 50% of the population). The dose ratio between therapeutic and toxic effects is the therapeutic index, and it can be expressed as the ratio, ED₅₀/LD₅₀. Pharmaceutical compositions that exhibit large therapeutic indices are preferred. The data obtained from cell culture assays and animal studies are used in formulating a range of dosage for human use. The dosage of such compounds lies preferably within a range of circulating concentrations that include the ED₅₀ with little or no toxicity. The dosage varies within this range depending upon the dosage form employed, sensitivity of the patient, and the route of administration.

The exact dosage is chosen by the individual physician in view of the patient to be treated. Dosage and administration are adjusted to provide sufficient levels of the active moiety or to maintain the desired effect. Additional factors that may be taken into account include the severity of the disease state, age, weight and gender of the patient; diet, time and frequency of administration, drug combination(s), reaction sensitivities, and tolerance/response to therapy. Long acting pharmaceutical compositions might be administered for example every 3 to 4 days, every week, once every two weeks, or once every three weeks, depending on half-life and clearance rate of the particular formulation.

Normal dosage amounts may vary from 0.1 to 100,000 micrograms, up to a total dose of about 10 g, depending upon the route of administration. Guidance as to particular dosages and methods of delivery is provided in the literature. See U.S. Pat. Nos. 4,657,760; 5,206,344; or 5,225,212.

### Kits

In a further aspect, the present disclosure relates to a kit comprising the isolated antibody or antigen-binding fragment according to the present disclosure or the conjugate according to the present disclosure and instructions for use. In particular embodiments, the kit comprises one or more containers filled with one or more of the ingredients of the aforementioned compositions of the disclosure. Associated with such container(s) can be a notice in the form prescribed by a governmental agency regulating the manufacture, use or sale of pharmaceuticals or biological products, reflecting approval by the agency of the manufacture, use or sale of the product for human administration.

### DRAWINGS

FIG. 1A: Effects of Sema3A inhibition with TPP-15370 (grey bar), TPP-11489 (striped bar) and TPP-17755 (squared bar) on Sema3A-induced albumin excretion in mice. Shown are mean±S.D. (n=10). ***, ****: p<0.001 p<0.0001 vs. isotype control. Dunnett's post hoc test.

FIG. 1B: Effects of Sema3A inhibition with TPP-23298 (grey bars), TPP-11489 (dotted bar) and TPP-17755 (striped bar) on Sema3A-induced albumin excretion in mice. Shown are mean±S.D. (n=10). ***, ****: p<0.001 p<0.0001 vs. isotype control. Dunnett's post hoc test.

FIG. 2A: Sema3A induced albuminuria in mice after treatment with TPP-15370 (white circles) and TPP-23298 (black circles) in comparison to TPP-11489 (black triangles). The comparisons were performed in two separate experiments. Shown are mean values. (n=10). n.s.=statistically not significant vs. TPP-15370; *=p<0.05 vs. TPP-23298. Unpaired T-test.

FIG. 2B: Sema3A induced albuminuria in mice after treatment with TPP-15370 (white circles) and TPP-23298 (black circles) in comparison to TPP-17755 (black squares). The comparisons were performed in two separate experiments. Shown are mean values. (n=10). n.s.=statistically not significant vs. TPP-15370; *=p<0.05 vs. TPP-23298. Unpaired T-test.

FIG. 2C: Sema3A induced albuminuria in mice after treatment with TPP-15370 (white circles) and TPP-23298 (black circles) in comparison to TPP-30788 (black rhombus). The comparisons were performed in two separate experiments. Shown are mean values. (n=10). n.s.=statistically not significant vs. TPP-15370; *=p<0.05 vs. TPP-23298. Unpaired T-test.

FIGS. 3A-3C: Effects of Sema3A inhibition with TPP-23374 (dotted bars) TPP-23298 (grey bars) and TPP-15370 (striped bars) on FIG. 3A: serum creatinine levels, FIG. 3B: serum urea levels and FIG. 3C: urinary albumin excretion after I/R injury in mice. Shown are mean±S.D. (n=8-10). *, **, ****: p<0.05, p<0.01, p<0.0001 vs. isotype control. Dunnett's post hoc test.

FIGS. 4A-4C: Effects of Sema3A inhibition with TPP-15370 (grey bars), TPP-11489 (striped bars) and TPP-17755 (squared bars) on FIG. 4A: serum creatinine levels, FIG. 4B: serum urea levels and FIG. 4C: urinary albumin excretion after I/R injury in mice. Shown are mean±S.D. (n=8-10). *, **, ****: p<0.05, p<0.01, p<0.0001 vs. isotype control. Dunnett's post hoc test.

FIGS. 5A-5C: Effects of Sema3A inhibition with TPP-23298 (grey bars), TPP-11489 (striped bars) and TPP-17755 (squared bars) on FIG. 5A: serum creatinine levels, FIG. 5B: serum urea levels and FIG. 5C: urinary albumin excretion after I/R injury in mice. Shown are mean±S.D. (n=10-12). *, **, ****: p<0.05, p<0.01, p<0.0001 vs. isotype control. Dunnett's post hoc test.

FIGS. 6A-6C: Effects of Sema3A inhibition with TPP-15374 (grey bars), TPP-11489 (striped bars) on FIG. 6A: serum creatinine levels, FIG. 6B: serum urea levels and FIG. 6C: urinary albumin excretion after I/R injury in mice. Shown are mean±S.D. (n=10-12). *, **, ***, ****: p<0.05, p<0.01, p<0.001 p<0.0001 vs. isotype control. Dunnett's post hoc test.

FIG. 7: Effects of Sema3A inhibition with TPP-15370 (grey bars), TPP-11489 (striped bars) on proteinuria in Alport mice. Shown are mean±S.D. (n=8-10). ****: p<0.0001 vs. isotype control. Dunnett's post hoc test.

FIGS. 8A-8D: Effects of Sema3A inhibition with TPP-15370 (grey bars), TPP-11489 (striped bars)) on FIG. 8A: serum creatinine levels, FIG. 8B: serum urea levels and fibrosis FIG. 8C: myofibroblast and FIG. 8D: collagen deposition in Alport mice. Shown are mean±S.D. (n=8-10). *, ***, ****: p<0.05, p<0.001, p<0.0001 vs. isotype control. Dunnett's post hoc test.

FIGS. 9A-9D: Effects of Sema3A inhibition with TPP-23298 in a single dose preventive setting in a unilateral kidney IRI model in pigs, 105 min of ischemia. TPP-23298 (FIG. 9A; black dots) or control IgG (open circles) (10 mg/kg) were given 30 min before inflating the balloon in the left renal artery. Values from SHAM animals are indicated diamonds. Time course of plasma creatinine concentrations of individual animals (FIG. 9A), and time course of mean change of creatinine plasma concentrations versus base line values at start of experimentation (0 h) (FIG. 9B). Mean values of creatinine clearance for 24-27 h interval. Creatinine clearance side separated for left (damaged) and right (non-damaged) kidneys and kidneys from sham animals (FIG. 9C). Global creatinine clearance (FIG. 9D); means±SEM, p-value in (FIG. 9B) from t-test, */*** in (FIG. 9C) and FIG. 9D): p<0.05/0.001, one-way ANOVA versus corresponding control followed by Dunnett's multiple comparison.

FIGS. 10A-10C: Schematic representation of a sandwich-based epitope binning experiment using SPR (see also Example 5A): FIG. 10A) One antibody is immobilized to a SPR chip, Sema3A is injected, and the binding is monitored; FIG. 10B) A second (competitive) antibody is injected on to the complex of the immobilized mAb bound to Sema3A, and the binding is monitored; FIG. 10C) A second (non-competitive) antibody is injected on to the complex of the immobilized mAb bound to Sema3A, and the binding is monitored.

FIGS. 11A-11C: HRA image analysis steps: FIG. 11A) Fluorescence microscopy image of DAPI/CM cells; FIG. 11B) Identification of cells in the selected area; FIG. 11C) Calculation of cells-free region size (grey area).

FIG. 12: The percent inhibition of Sema3A in a HUVEC repulsion assay at an antibody concentration of 80 pM is shown (see Example 11). Each column represents one antibody in the following left to right order: TPP-23298 (black column), TPP-30788, TPP-TPP-30789, TPP-30790, and TPP-30791.

### EXAMPLES

### Example 1: Sema3A Sequences and Tool Generation

TABLE 2
Tools used in this disclosure

| | | Bounderies | | |
|---|---|---|---|---|
| TPP-No. | Protein | [aa] | Uniprot ID | Catalog No. |
| TPP-13211 | Human Semaphorin3A-Fc (R&D | 26-771 | Q14563 | 1250-S3 |
| | Systems) | | | |
| No TPP-No. | Human Semaphorin3G (Abnova) | 1-782 | Q9NS98 | H00056920- |
| | | | | P01 |
| No TPP-No. | Human Semaphorin3F-Fc (R&D | 19-772 | Q13275 | 9878-S3 |
| | Systems) | | | |
| TPP-13357 | Mouse Semaphorin3A-Fc (R&D | 21-747 | O08665 | 5926-S3 |
| | Systems) | | | |
| TPP-19068 | Human Semaphorin3A-Sema | 21-569 | Q14563 | Produced |
| | Domain | | | inhouse |
| TPP-19069 | Mouse Semaphorin3A-Sema | 21-569 | O08665 | Produced |
| | Domain | | | inhouse |
| TPP-19122 | Cyno Semaphorin3A-Sema | 21-569 | Q63548 | Produced |
| | Domain | | | inhouse |
| TPP-19120 | Rat Semaphorin3A-Sema | 21-569 | E2QX94 | Produced |
| | Domain | | | inhouse |
| TPP-19121 | Dog Semaphorin3A-Sema | 21-569 | A0A2K5VGJ0 | Produced |
| | Domain | | | inhouse |
| TPP-20176 | Pig Semaphorin3A-Sema | 49-658 | A0A480WHT2 | Produced |
| | Domain | | | inhouse |

Sema3A domains were produced by mammalian cell culture using transiently transfected HEK293-6E cells (National Research Council Canada). All constructs were under the control of a CMV promoter and sequences contain a C-terminal FXa cleavage site followed by a 6×his-tag. Cell culture was performed using F17 medium (Life Technologies) supplemented with 0.1% pluronic F68 (Life Technologies) and 4 mM Glutamax (Life Technologies). 24 h post-transfection, 1% FCS ultra-low IgG (Life Technologies) and 0.5 mM valproic acid (Sigma Aldrich) were added. Cell supernatant was sterile filtered and subsequently purified or concentrated via crossflow filtration prior to purification.

Sema3A domains were purified using a two-step purification consisting of affinity and size exclusion chromatography. In brief, cell culture supernatant was loaded on to a Ni²⁺-NTA column (GE Healthcare) connected to an Äkta Avant system (GE Healthcare). Column was equilibrated with 4 CV of 50 mM NaH₂PO₄, 300 mM NaCl, pH 8 and washed afterwards with 10 CV of running buffer until baseline was reached. Elution was carried out using 6 CV of running buffer containing 250 mM imidazole, pH 8.0. Fractions of the elution peak were unified, concentrated using a Vivaflow 200 Hydrosart membrane (cut-off 10 kDa, Sartorius) and subjected to size exclusion chromatography using a Superdex 200 column (GE Healthcare) connected to an Äkta Pure 25 system. The column was equilibrated and run in DPBS, pH 7.4. Fractions of the domain elution peak were unified and concentrated using a Vivaflow 200 Hydrosart membrane (cut-off 10 kDa, Sartorius). The final protein quality was assessed on an analytical size exclusion chromatography (Superdex 200) for purity and monodispersity as well as SDS-PAGE. Sema domains were aliquoted and snap frozen in liquid nitrogen and stored at −80° C. until further use.

### Example 2: Antibody Generation from BioInvent Antibody Libraries

A fully human antibody phage display library (BioInvent n-CoDeR Fab lambda library) was used to isolate human monoclonal antibodies of the present disclosure by selection against recombinant human Sema3A (TPP-13211, R&D Systems) using the following protocol. Briefly, Immunotubes (Nunc) were coated for one hour at room temperature (RT) with the 100 μg of the target molecule (huSema3A) or an irrelevant Fc-containing off-target in 1 ml PBS (Phosphate Buffered Saline) with end-over-end rotation. The target and depletion antigen-coated immunotube as well as an empty immunotube were washed 4 times with PBS+0.05% Tween20 (PBST) and subsequently blocked using 3 ml of a 3% Milk powder in PBST solution for 1 h at RT with end-over-end rotation. An aliquot of the phage library was thawed and allowed to block in a solution of 3% milk powder in PBST for 1 h at RT with end-over-end rotation. The non-coated depletion immunotube was washed 3 times in 4 ml PBS before addition of the blocked phage library and incubation with end-over-end rotation for 30 min at RT. This step was repeated for the non-target antigen-coated depletion immunotube. The huSema3A-coated immunotube was washed 3 times in 4 ml PBS before addition of the depleted library and incubation for 90 min at room temperature with end-over-end rotation. After stringent washing (4× with 4 ml PBST and 1× with 4 ml PBS) Fab-expressing phages binding specifically to the coated target were eluted using 500 μl 100 nM TEA, 10 min incubation at room temperature followed by neutralization by addition of 500 μl Tris-HCl pH 7.5. 500 μl of eluted phage were used to infect Escherichia coli strain HB101. Subsequently the phages were amplified in Escherichia coli strain HB101 using M13K07 Helper Phage (Invitrogen™). In two subsequent selection rounds the target concentration was decreased to 25 μg/ml. For a first qualitative assessment, 88 randomly picked Fab-expressing phage clones from each selection round were expressed in single wells and tested for binding to huSema3A compared to an irrelevant off-target. The clone pool from Round 3 in this example was found to contain a 60% positive hit rate and was chosen for further screening.

In a next step, the expression of soluble Fabs was enabled by bulk removal of the gene III fusion in this pool and 2208 single clones were picked for expression in Escherichia coli strain Top10 and evaluation of Fab-containing supernatants in a huSema3A binding ELISA. The VH and VL sequences for all 2208 clones was also determined using NGS methods. 154 distinct clones positive for binding to huSema3A were identified. These positive binding Fab fragments were tested in a confirmatory binding ELISA and were also evaluated for binding to mouse Sema3A-Fc (TPP-13357, R&D Systems) as well as specificity testing using an additional off target molecule, murine Sema3F (R&D Systems). Based on this analysis, 48 human/mouse cross-reactive Sema3A binding Fabs were prioritized. These Fab fragments were subsequently purified from 25 ml expression cultures using Capture Select CH1 matrix (LifeTechnologies), eluted using 12.5 mM Citric acid at pH 2.5 and finally buffer exchanged to PBS using a Zeba™ Spin desalting plate (ThermoFisher). A kinetic ranking was performed for all 48 purified Fab fragments by surface plasmon resonance (SPR), examining the binding to both human and mouse Sema3A and reformatted in to a full-length human IgG1 and again tested for binding in SPR (see Example 4).

### Example 3: Sequence Optimization, Germlining & Affinity Maturation of Lead Antibodies TPP-15370 and TPP-15374

IgG1 antibodies TPP-15370 and TPP-15374 were subjected to lead optimization procedures aiming to (i) optimize its affinity, (ii) increase functional efficiency, (iii) reduce the risk of sequence-based immunogenicity and (iv) improve compatibility with downstream development processes.

Affinity maturation was done by a first single mutation gathering round followed by recombination of the most affinity- and potency-increasing amino acid exchanges in a germlined and sequence optimized antibody backbone.

For mutation gathering NNK (N=A or G or C or T, K=G or T) randomizations at the following individual amino acid positions were generated by site directed mutagenesis using synthetic oligonucleotides including NNK for codon-diversification. For TPP-15370 the following regions were analyzed for their effect on affinity: GFTFSSYGMH (residues 26 to 35 of VH SEQ ID NO: 41), WVSAIGTGGDTYYADSVMG (residues 47 to 65 of VH SEQ ID NO: 41), ARRDDYTSRDAFDV (residues 96 to 109 of VH SEQ ID NO:41), SGSSSNIGSNTVNWY (residues 23 to 37 of VL SEQ ID NO: 45), LLIYYDDLLPS (residues 47 to 57 of VL SEQ ID NO: 45), and AAWDDSLNGYVV (residues 90 to 101 of VL SEQ ID NO: 45).

For TPP-15374 the following regions were analyzed for their effect on affinity: GFTFSSYEMN (residues 26 to 35 ofVH SEQ ID NO: 61), WVSGISWNSGSIGYADSVKG (residues 47 to 66 of VH SEQ ID NO: 61), ARSGYSSSWFDPDFDY (residues 97 to 112 of VH SEQ ID NO: 61), TGSSSNIGAGYDVHWY (residues 23 to 38 of VL SEQ ID NO: 65), LLIYGNSNRPS (residues 48 to 58 of VL SEQ ID NO: 65), and SSYAGSNPYV (residues 91 to 101 of VL SEQ ID NO: 65).

The resulting single NNK libraries were sequenced and about 1000 single amino acid exchange variants of TPP15370 and TPP-15374, respectively, were identified. They were expressed by transient transfection of mammalian cells and resulting expression supernatants were normalized in terms of antibody concentrations to be screened in surface plasmon resonance and competition ELISA.

For the germlining and sequence optimization process of TPP-15370 and TPP-15374 the closest germline families for light and heavy chain were selected and scrutinized for potential CMC relevant residues. Deviations from closest human germlines in CDR regions and FW regions and potential CMC relevant residues in CDR regions were adjusted by site directed mutagenesis and tested for in functional and biophysical assays (unspecific binding, temperature stability in DSC). The resulting single reversions and following combinatorial IgG variants were expressed by transient transfection of mammalian cells and resulting expression supernatants were normalized in terms of antibody concentrations to be screened in binding assays (SPR, competition ELISA) and functional assays. This led to germlined and sequence optimized molecules TPP-21565 for TPP-15370 and TPP-18533 for TPP-15374. TPP-21565 carries in comparison to TPP-15370 reversions L55R and R80Q in the light chain and G33A, H35S, M64K and V109Y in the heavy chain. TPP-18533 carries in comparison to TPP-15374 reversions A10V, T13A, S78T, R81Q, S82A in the light chain.

For the final recombination library of TPP-21565 eight single substitution variants that were shown in the NNK library screening step to exhibit improved affinity and functional efficiency were selected. Light chain mutations A90H, G98D, G98V, Y99I and V100P and heavy chain mutations S30Y, S35L and T53Y were recombined in one recombination library (continuous amino acid nomenclature, reference is TPP-21565 as defined by SEQ ID NOs: 121—VH and 125—VL).

For the final recombination library of TPP-18533 eleven single substitution variants that were shown in the NNK library screening step to exhibit improved affinity and functional efficiency were selected. Light chain mutations N28D, N53A, S91K, S91Q, A94E, S961 and S96P and heavy chain mutations T28D, S30D, S57W and G59Y were recombined in one recombination library (continuous amino acid nomenclature, reference is TPP-18533 as defined by SEQ ID NOs: 101—VH and 105—VL).

For TPP-18533 oligonucleotides were generated to introduce selected mutations or the corresponding wild type amino acid at each selected position. Library construction was performed using sequential rounds of overlap extension PCR. The final PCR product was ligated into a mammalian IgG4 (S228P) expression vector and variants were sequenced using massive-parallel sequencing techniques. For TPP-21565 the recombinatorial variants were designed as distinct clones and cloned into an IgG4 (S228P) containing expression plasmid.

More than 1000 unique combinatorial amino acid exchange variants of TPP-18533 and more than 100 unique combinatorial variants of TPP-21565 were generated in that way, expressed by transient transfection of mammalian cells, and resulting expression supernatants were normalized in terms of antibody concentrations to be screened in varying number in SPR, competition ELISA and functional assays. Based on the result in these assays, mutants were either categorized as ‘improved’ or ‘non-improved’.

Table 1 and 1A lists i.a. preferred antibodies candidates according to the present disclosure that were selected in the combination library screening step as being most potent in terms of binding to Sema3A and in terms of antagonizing the Sema3A-dependent biological activity as well as the respective amino acid and nucleic acid sequences of antibodies according to the present disclosure.

### Example 4: Determination of Affinity and Species Cross-Reactivity Using Surface Plasmon Resonance

To assess the binding kinetics and affinity of anti-Sema3A antibodies as well as their species cross-reactivity profile, binding assays were conducted using surface plasmon resonance (SPR). Binding assays were performed on a Biacore T200 instrument or on a Biacore 8K+ instrument (Cytiva) at 25° C. using assay buffer HBS P+, 300 mM NaCl, 0.75 mM CaCl₂, 2.5 mM MgCl₂, 1 mg/ml BSA, 0.05% NaN₃. Antibodies were captured either via anti-human Fc IgGs (“Human antibody capture kit”, Order No. BR100839, Cytiva) or in case of Fc-tagged analytes by anti-human Fab IgGs (“Human Fab capture kit”, Order No. 28958325, Cytiva) covalently amine coupled to a Series S CM5 sensor chip (Cytiva). The amine coupling was carried out according to the manufacturer's instructions using 1-ethyl-3-(3-dimethylaminopropyl) carbodiimide hydrochloride (EDC), N-hydroxysuccinimide (NHS) and ethanolamine HCl, pH 8.5 (“Amine Coupling Kit” BR-1000-50, Cytiva.). For phage display hits Fc-tagged human and mouse Sema3A was used as analytes in a concentration range from 1.56-200 nM. Human, mouse, cynomolgus, rat, dog and pig monovalent Sema3A domain were used as analytes in a concentration series from 0.0.024-3.125 nM in multi cycle kinetics mode or in 100 nM for binding analysis only. The sensor surface was regenerated with glycine pH 2.0 after each antigen injection. Obtained sensorgrams were double referenced (subtraction of reference flow cell signal and buffer injection) and were fitted to a 1:1 Langmuir binding model to derive kinetic data using the Biacore T200 Evaluation software. Results are shown in Tables 3, 4 and 4a.

TABLE 3
Affinity of anti-Sema3A IgG1 antibodies derived from phage
display hits determined by SPR using TPP-13211 and TPP-13357.

| | Mouse | Human |
|---|---|---|
| Nomenclature | K_{D} [M] | K_{D} [M] |
| TPP-15355 | 4.0E−09 | 3.5E−09 |
| TPP-15356 | n.b. | 3.2E−09 |
| TPP-15357 | 1.0E−07 | 5.0E−08 |
| TPP-15358 | 3.1E−09 | 9.5E−10 |
| TPP-15359 | n.b | 1.1E−08 |
| TPP-15360 | 1.1E−07 | 7.2E−09 |
| TPP-15361 | n.b. | 5.5E−09 |
| TPP-15362 | n.b. | n.b. |
| TPP-15363 | n.b. | 2.4E−09 |
| TPP-15364 | n.b. | 2.6E−09 |
| TPP-15365 | 2.4E−07 | 6.5E−08 |
| TPP-15366 | 1.4E−08 | 1.3E−08 |
| TPP-15367 | 5.4E−09 | 2.2E−09 |
| TPP-15368 | 8.2E−07 | 1.5E−07 |
| TPP-15369 | 4.1E−08 | 3.5E−08 |
| TPP-15370 | 3.2E−09 | 2.8E−09 |
| TPP-15371 | 7.4E−09 | 4.5E−09 |
| TPP-15372 | n.b. | 3.7E−09 |
| TPP-15373 | 2.0E−07 | 1.3E−07 |
| TPP-15374 | 1.8E−08 | 1.8E−08 |
| TPP-15375 | 5.8E−09 | 5.2E−09 |
| TPP-15376 | 8.4E−09 | 5.8E−09 |
| TPP-15377 | 3.3E−09 | 1.9E−09 |
| TPP-15378 | n.d. | 1.2E−08 |
| TPP-15379 | 4.3E−07 | 2.1E−07 |
| TPP-15380 | n.b. | n.b. |
| TPP-15381 | 9.9E−09 | 3.3E−09 |
| TPP-15382 | 2.5E−07 | 1.9E−07 |
| TPP-15383 | 5.3E−08 | 2.8E−08 |
| TPP-15384 | 9.6E−09 | 9.1E−09 |
| TPP-15385 | 8.5E−09 | 7.2E−09 |
| TPP-15386 | n.b. | n.b. |
| TPP-15387 | 1.6E−07 | 1.1E−07 |
| TPP-15388 | 1.7E−07 | 1.3E−08 |
| TPP-15389 | 4.2E−09 | 2.8E−09 |
| TPP-15390 | 9.8E−08 | 5.7E−08 |
| TPP-15391 | n.b. | 7.0E−09 |
| TPP-15392 | n.d. | n.d. |
| TPP-15393 | 5.9E−08 | 9.3E−09 |
| TPP-15394 | n.d. | n.d. |
| TPP-15395 | 1.1E−06 | 2.2E−07 |
| TPP-15396 | 6.2E−09 | 2.1E−09 |
| TPP-15397 | 2.7E−07 | 9.7E−09 |
| TPP-15398 | 8.5E−09 | 8.4E−09 |
| TPP-15399 | 1.9E−07 | 1.5E−07 |
| TPP-15400 | 4.9E−09 | 4.6E−09 |
| TPP-15401 | 7.6E−07 | 1.2E−08 |

n.b. = no binding,
n.d. = not determinable

The majority of phage display hits bind to human and mouse dimeric Sema3A in the lower nanomolar range.

TABLE 4
Affinity of anti-Sema3A antibodies derived from TPP-15370 and TPP-
15374 determined by SPR using TPP-19068, TPP-19069, TPP-19122,
TPP-19120, TPP-19121, TPP-20176 as analytes as well as prior art
antibodies (TPP-30972 was purified from HEK cell expression).

| | Mouse | Pig | Cyno | Dog | Human | Rat |
|---|---|---|---|---|---|---|
| Nomenclature | K_{D} [M] | K_{D} [M] | K_{D} [M] | K_{D} [M] | K_{D} [M] | K_{D} [M] |
| TPP-11489 | 1.6E−07 | 1.0E−07 | 6.3E−08 | 5.0E−08 | 7.3E−08 | 3.3E−08 |
| (Chiome) | | | | | | |
| TPP-17755 | 3.9E−09 | 4.0E−09 | 1.4E−08 | 7.5E−09 | 6.9E−09 | 5.4E−09 |
| (Samsung) | | | | | | |
| TPP-30791 | 2.8E−11 | 2.9E−11 | 5.7E−11 | 7.8E−11 | 1.2E−11 | 1.7E−11 |
| (BI clone IV) | | | | | | |
| TPP-30790 | 4.0E−11 | 3.6E−11 | 7.7E−11 | 1.0E−10 | 1.5E−11 | 2.2E−11 |
| (BI clone III) | | | | | | |
| TPP-30789 | 4.2E−11 | 3.9E−11 | 7.9E−11 | 1.1E−10 | 2.2E−11 | 2.6E−11 |
| (BI clone II) | | | | | | |
| TPP-30788 | 4.3E−11 | 3.8E−11 | 7.8E−11 | 1.1E−10 | 1.8E−11 | 2.6E−11 |
| (BI clone I) | | | | | | |
| TPP-30792 | | | | | | |
| (3H4 Univ | no binding | no binding | no binding | no binding | no binding | no binding |
| Ramot) | | | | | | |
| TPP-15370 | 7.2E−09 | 9.0E−09 | 4.0E−08 | 2.2E−08 | 1.0E−08 | 1.4E−08 |
| TPP-23298 | 7.4E−11 | 6.7E−11 | 7.8E−11 | 7.0E−11 | 8.7E−11 | 3.0E−11 |
| TPP-23334 | 6.2E−11 | 1.4E−11 | 1.5E−11 | 8.4E−12 | 2.1E−11 | 5.6E−11 |
| TPP-23337 | 5.0E−11 | 1.1E−11 | 2.6E−11 | 4.5E−12 | 5.0E−11 | 1.1E−10 |
| TPP-23338 | 4.5E−11 | 4.6E−11 | 4.2E−11 | 5.3E−11 | 5.4E−11 | |
| TPP-23340 | 5.9E−11 | 6.2E−11 | 6.0E−11 | 5.8E−11 | 2.2E−11 | |
| TPP-23341 | 9.2E−11 | 8.6E−11 | 8.7E−11 | 8.4E−11 | 9.1E−11 | |
| TPP-23345 | 6.3E−11 | 5.5E−11 | 6.2E−11 | 4.6E−11 | 6.5E−11 | |
| TPP-23346 | 6.4E−11 | 5.8E−11 | 6.1E−11 | 6.1E−11 | 7.2E−11 | |
| TPP-23347 | 5.5E−11 | 5.3E−11 | 5.4E−11 | 5.1E−11 | 6.0E−11 | |
| TPP-23373 | 8.3E−11 | 7.8E−11 | 7.2E−11 | 1.0E−10 | 1.1E−10 | |
| TPP-23374 | 1.6E−11 | below 3 pM | below 3 pM | 7.3E−12 | 8.1E−12 | 3.3E−12 |
| TPP-23375 | 4.2E−11 | 4.7E−11 | 4.5E−11 | 4.5E−11 | 5.3E−11 | |
| TPP-15374 | 8.3E−09 | 7.2E−09 | 4.6E−08 | 1.9E−08 | 1.5E−08 | 9.8E−09 |
| TPP-18533 | 8.1E−09 | | 6.4E−09 | | 8.7E−09 | |
| TPP-25497 | | | | | 5.2E−11 | |
| TPP-25256 | | | | | 4.9E−11 | |
| TPP-25255 | | | | | 5.1E−11 | |
| TPP-25257 | | | | | 5.3E−11 | |
| TPP-25248 | | | | | 5.0E−11 | |
| TPP-25064 | | | | | 4.9E−11 | |
| TPP-26111 | | | | | 5.2E−11 | |
| TPP-25224 | | | | | 4.9E−11 | |
| TPP-25448 | | | | | 5.3E−11 | |
| TPP-25655 | | | | | 4.9E−11 | |

All derivative antibodies of TPP-15370 and TPP-15374 have a significantly increased affinity to the Sema3A domain in the lower picomolar range compared to their parental antibodies as well as to most prior art antibodies.

TABLE 4a
Affinity of anti-Sema3A IgG1 antibodies determined by SPR using
100 nM TPP-19068 (human) in a binding experiment.

| | Human |
|---|---|
| Nomenclature | K_{D} [M] |
| TPP-23298 | 1.3E−10 |
| TPP-17755 (Samsung) | 6.2E−09 |
| TPP-11489 (Chiome) | n.d. |
| TPP-30788 (BI clone I) | 9.8E−11 |
| TPP-31357 (Fab of 3H4 Univ Ramot) | 3.5E−10 |

n.d. = not determinable due to multiphasic behaviour

In contrast to the full length 3H4 IgG1 (TPP-30792) which showed no binding in SPR to Sema3A molecules, the Fab variant of TPP-30792, TPP-31357 shows binding to human Sema3A, but with less affinity as TPP-23298.

### Example 5: Determination of Binding Activity Using Surface Plasmon Resonance

To assess the binding activity of anti-Sema3A antibodies binding assays were conducted using surface plasmon resonance (SPR). Binding assays were performed on a Biacore T200 instrument (Cytiva) at 25° C. using assay buffer HBS P+, 300 mM NaCl, 0.75 mM CaCl₂, 2.5 mM MgCl₂, 1 mg/ml BSA, 0.05% NaN₃. Antibodies were captured via anti-human Fc IgGs (“Human antibody capture kit”, Order No. BR100839, Cytiva) covalently amine coupled to a Series S CM5 sensor chip (Cytiva). The amine coupling was carried out according to the manufacturer's instructions using 1-ethyl-3-(3-dimethylaminopropyl) carbodiimide hydrochloride (EDC), N-hydroxysuccinimide (NHS) and ethanolamine HCl, pH 8.5 (“Amine Coupling Kit” BR-1000-50, Cytiva.). Human, mouse, cynomolgus, rat, dog and pig monovalent Sema3A domain were used as analytes in a concentration series from 0.024-3.125 nM in multi cycle kinetics mode. The sensor surface was regenerated with glycine pH 2.0 after each antigen injection. Obtained sensorgrams were double referenced (subtraction of reference flow cell signal and buffer injection) and were fitted to a 1:1 Langmuir binding model using the Biacore T200 Evaluation software obtaining the experimental fitted R_{Max} value. To calculate the binding activity first the theoretical R_{Max} needs to be calculated according to equation 1:

$$\\ {R}_{Max} = \frac{{R}_{Ligand} * {Mr}_{Analyte} * {Valency}_{Ligand}}{{Mr}_{Ligand}}$$

Equation 1: Theoretical calculation of R_{Max}. R_{Ligand}=Ligand Level in RU, Mr=molecular weight, Valency_{Ligand}=number of binding sites per antibody molecule, here 2

Binding activity was determined by dividing the experimental determined R_{Max} by the theoretical calculated R_{Max} according to equation 2:

$$\\ Activity in \% = \frac{{R}_{Max experimental}}{{R}_{Max theoretical}} * 100$$

Equation 2: Calculation of binding activity in %

TABLE 5
Summary of ligand levels after capture, experimental, theoretical
and binding activity of tested antibodies

| | | Ligand | | Theoretical | Binding |
|---|---|---|---|---|---|
| | | Level | Experimental | Rmax | Activity |
| Ligand | Analyte | [RU] | Rmax [RU] | [RU] | [%] |
| TPP-11489 | Rat | 798 | 212 | 681 | 31 |
| (Chiome) | Sema3A | | | | |
| TPP-15370 | domain | 53 | 51 | 45 | 113 |
| TPP-15374 | | 53 | 42 | 45 | 94 |
| TPP-17755 | | 54 | 26 | 46 | 56 |
| (Samsung) | | | | | |
| TPP-23298 | | 46 | 42 | 39 | 108 |
| TPP-30791 | | 46 | 42 | 39 | 109 |
| (BI clone IV) | | | | | |
| TPP-30790 | | 62 | 50 | 53 | 94 |
| (BI clone III) | | | | | |
| TPP-30789 | | 50 | 44 | 42 | 104 |
| (BI clone II) | | | | | |
| TPP-30788 | | 46 | 43 | 40 | 109 |
| (BI clone I) | | | | | |
| TPP-11489 | Dog | 797 | 148 | 680 | 22 |
| (Chiome) | Sema3A | | | | |
| TPP-15370 | domain | 53 | 51 | 45 | 114 |
| TPP-15374 | | 53 | 44 | 45 | 98 |
| TPP-17755 | | 54 | 25 | 46 | 55 |
| (Samsung) | | | | | |
| TPP-23298 | | 45 | 42 | 39 | 107 |
| TPP-30791 | | 47 | 43 | 40 | 106 |
| (BI clone IV) | | | | | |
| TPP-30790 | | 61 | 48 | 52 | 92 |
| (BI clone III) | | | | | |
| TPP-30789 | | 50 | 44 | 43 | 102 |
| (BI clone II) | | | | | |
| TPP-30788 | | 47 | 42 | 40 | 106 |
| (BI clone I) | | | | | |
| TPP-11489 | Pig | 801 | 525 | 684 | 77 |
| (Chiome) | Sema3A | | | | |
| TPP-15370 | domain | 53 | 50 | 45 | 111 |
| TPP-15374 | | 53 | 47 | 45 | 103 |
| TPP-17755 | | 54 | 28 | 46 | 60 |
| (Samsung) | | | | | |
| TPP-23298 | | 46 | 42 | 39 | 107 |
| TPP-30791 | | 49 | 44 | 42 | 105 |
| (BI clone IV) | | | | | |
| TPP-30790 | | 61 | 48 | 52 | 92 |
| (BI clone III) | | | | | |
| TPP-30789 | | 51 | 45 | 43 | 103 |
| (BI clone II) | | | | | |
| TPP-30788 | | 47 | 43 | 40 | 107 |
| (BI clone I) | | | | | |
| TPP-11489 | Cyno | 800 | 85 | 682 | 13 |
| (Chiome) | Sema3A | | | | |
| TPP-15370 | domain | 53 | 63 | 45 | 139 |
| TPP-15374 | | 53 | 47 | 45 | 104 |
| TPP-17755 | | 53 | 24 | 45 | 53 |
| (Samsung) | | | | | |
| TPP-23298 | | 46 | 41 | 39 | 106 |
| TPP-30791 | | 47 | 43 | 40 | 107 |
| (BI clone IV) | | | | | |
| TPP-30790 | | 62 | 48 | 52 | 92 |
| (BI clone III) | | | | | |
| TPP-30789 | | 50 | 44 | 42 | 103 |
| (BI clone II) | | | | | |
| TPP-30788 | | 47 | 43 | 40 | 107 |
| (BI clone I) | | | | | |
| TPP-11489 | Human | 798 | 257 | 681 | 38 |
| (Chiome) | Sema3A | | | | |
| TPP-15370 | domain | 53 | 51 | 45 | 112 |
| TPP-15374 | | 53 | 45 | 45 | 10 |
| TPP-17755 | | 54 | 25 | 46 | 55 |
| (Samsung) | | | | | |
| TPP-23298 | | 46 | 42 | 39 | 107 |
| TPP-30791 | | 48 | 44 | 41 | 107 |
| (BI clone IV) | | | | | |
| TPP-30790 | | 61 | 48 | 52 | 93 |
| (BI clone III) | | | | | |
| TPP-30789 | | 49 | 45 | 42 | 106 |
| (BI clone II) | | | | | |
| TPP-30788 | | 47 | 43 | 40 | 107 |
| (BI clone I) | | | | | |
| TPP-11489 | Mouse | 796 | 803 | 680 | 118 |
| (Chiome) | Sema3A | | | | |
| TPP-15370 | domain | 53 | 50 | 45 | 11. |
| TPP-15374 | | 53 | 48 | 45 | 106 |
| TPP-17755 | | 54 | 26 | 46 | 57 |
| (Samsung) | | | | | |
| TPP-23298 | | 46 | 42 | 39 | 108 |
| TPP-30791 | | 47 | 43 | 40 | 107 |
| (BI clone IV) | | | | | |
| TPP-30790 | | 62 | 49 | 52 | 93 |
| (BI clone III) | | | | | |
| TPP-30789 | | 51 | 45 | 43 | 103 |
| (BI clone II) | | | | | |
| TPP-30788 | | 47 | 43 | 40 | 108 |
| (BI clone I) | | | | | |

The binding activity calculated in the SPR experiment is a measure of the activity of the surface-attached ligand. As can be seen from Table 5, TPP-15370, TPP-15374, TPP-23298 and TPPs 30788-30791 are able to bind to all tested Sema3A domains with around 100% activity meaning all binding regions are fully able to bind. Prior art antibody TPP-17755 only reaches an activity level of 50-60% depending on the species. Prior art antibody TPP-11489 shows an even more reduced level of below 50%, except for mouse and pig where it is higher. Strikingly, to reach such an activity level, the ligand level of TPP-11489 needs to be over 10-fold higher as compared to the other antibodies pointing in general to a much lower binding activity as compared to TPP-15370, TPP-15374 and TPP-23298.

### Example 6: Competition ELISA

For screening in a competition ELISA setup, human Sema3a (TPP-13211) was coated onto 384-well plates (Greiner bio-one, 781077) with a concentration of 0.5 μg/ml in coating buffer (Carbonate-Basis pH 9.6, Candor 121125) over night at 10° C. After washing the plates 3 times with 50 μl PBS 0.05% Tween the plates were blocked with 50 μl Smart Block® (Candor 113500) for 1 h at 20° C. and washed again 3 times as described.

Subsequently, 20 μl of pre-mixed antibody solution was added to the plates and incubate for 18 h at 10° C. For the pre-mixed antibody solution, for each well, one biotinylated, parental antibody being either TPP-15370 or TPP-15374 was mixed in a ratio 1:1, 1:5 or 5:1 with an antibody containing one or more amino acid variations within its CDR regions (recombination variants) and not containing any biotin tag. As additional controls an isotype control antibody not demonstrating any binding to human Sema3A was also used as competition antibody. The total concentration of the added antibody solution was 0.25 μg/ml. During the incubation time the antibodies bound to the plates in a competitive manner as they compete for the same epitope on the human Sema3A protein.

After subsequent washing with 50 μl PBS 0.05% Tween for 3 times, 20 μl of a Streptavidin-HRP solution (R&D Systems, DY998, 1:200 in PBS 0.05% Tween 10% Smart Block) were added and incubated for 1 h and 20° C. followed by subsequent washing 3 times with 50 μl PBS 0.05% Tween and addition of 20 μl Amplex Red solution (Invitrogen A12222, 1:1000 in NaP-buffer 50 mM pH7.6 with 1:10000 of 30% H2O2). After a final incubation for 20 min at 20° C. the signal was determined using an emission wavelength of 595 nm and excitation of 530 nm. Due to the biotinylation of the parental antibodies TPP-15370 and TPP-15374 only the binding of these variants can be detected. Hence, competition with an antibody variant demonstrating superior binding shows a lower binding signal in comparison to e.g. competition of the parental antibody with a non-bioinylated version of itself.

In total, 103 recombination variants of TPP-15370 and 1136 recombination variants for TPP-15374 were measured. For analysis, and to allow for correction of plate-to-plate variations, the ELISA raw values were normalized to the value of the competition with the isotype control antibody TPP-9809.

Table 6 lists the values for the competition ELISA for selected recombination variants of TPP-15370 and TPP-15374. Depicted are the ratios vs. the isotype control antibody TPP-9809 in the measurement with a 1 to 5 or a 1 to 1 ratio, respectively.

TABLE 6
Values for the competition ELISA for recombination variants of TPP-15374 and TPP-15370.
Depicted are the ratios vs. the isotype control antibody for selected recombination variants, respectively,
when normalized to the isotype control antibody TPP-9809 in the measurement with a 1 to 5 or a 1 to
1 ratio, respectively.

| TPP-15374 family | | | TPP-15370 family | | |
|---|---|---|---|---|---|
| | VAL norm to | VAL norm to | | VAL norm to | VAL norm to |
| TPP | TPP-9809 | TPP-9809 | TPP | TPP-9809 | TPP-9809 |
| Number | (1 to 5 ratio) | (1 to 1 ratio) | Number | (1 to 5 ratio) | (1 to 1 ratio) |
| TPP-15374 | 0.41 | 0.69 | TPP-15370 | 0.54 | 0.67 |
| TPP-9809 | 1.00 | 1.00 | TPP-9809 | 1.00 | 1.00 |
| TPP-25497 | 0.26 | 0.39 | TPP-23298 | 0.09 | 0.18 |
| TPP-25256 | 0.15 | 0.41 | TPP-23334 | 0.11 | 0.28 |
| TPP-25255 | 0.17 | 0.37 | TPP-23337 | 0.14 | 0.27 |
| TPP-25257 | 0.18 | 0.36 | TPP-23338 | | 0.33 |
| TPP-25248 | 0.20 | 0.36 | TPP-23340 | | 0.40 |
| TPP-25064 | 0.19 | 0.48 | TPP-23341 | 0.18 | 0.38 |
| TPP-26111 | 0.18 | 0.49 | TPP-23345 | 0.08 | 0.27 |
| TPP-25224 | 0.17 | 0.43 | TPP-23346 | 0.13 | 0.22 |
| TPP-25448 | 0.19 | 0.47 | TPP-23347 | 0.16 | 0.30 |
| TPP-25655 | 0.23 | 0.39 | TPP-23373 | 0.20 | 0.35 |
| | | | TPP-23374 | 0.08 | 0.19 |
| | | | TPP-23375 | 0.16 | 0.30 |

### Example 5a: Epitope Binning Using Surface Plasmon Resonance (SPR)

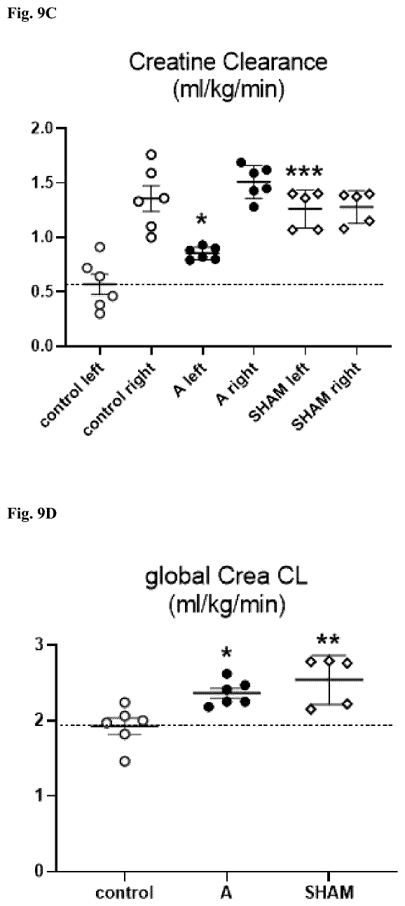

An epitope binning experiment was performed to determine the epitope bins of anti-Sema3A antibodies using SPR by employing a classical sandwich approach. In this experiment, one antibody is immobilized to a SPR chip, Sema3A is injected, and the binding is monitored (FIG. 10A). After successful binding of Sema3A to the first antibody, a second antibody is injected on to the complex of the immobilized mAb bound to Sema3A and the additional binding is monitored (FIG. 10B and FIG. 10C). If the second antibody competes with the first antibody for the binding to Sema3A than no additional binding signal is detected after injection of the second antibody, showing that the two antibodies bind to the same or very adjacent Sema3A epitope (FIG. 10C). If the second antibody does not compete with the first antibody for the binding to Sema3A than an additional binding signal is detected after injection of the second antibody, showing that the two antibodies bind to different Sema3A epitopes (FIG. 10B).

Experiments were performed on a Biacore T200 instrument (Cytiva) at 25° C. using assay buffer HBS P+, 300 mM NaCl, 0.75 mM CaCl₂, 2.5 mM MgCl₂, 1 mg/ml BSA, 0.05% NaN₃. Antibodies were covalently amine coupled to a Series S CM5 sensor chip (Cytiva). The amine coupling was carried out according to the manufacturer's instructions using 1-ethyl-3-(3-dimethylaminopropyl) carbodiimide hydrochloride (EDC), N-hydroxysuccinimide (NHS) and ethanolamine HCl, pH 8.5 (“Amine Coupling Kit” BR-1000-50, Cytiva.). Human, monovalent Sema3A domain was used as first analyte in a concentration of 200 nM followed by a second injection of the competitor antibody. This setup was performed with all possible combinations. The sensor surface was regenerated with glycine pH 2.0 after each antigen injection. Table 6a shows the binning results.

TABLE 6a
Matrix view of the epitope binning results
(+ = additional binding, − = no additional binding)

| First antibody/ | TPP-30788 | | TPP-17755 |
|---|---|---|---|
| second antibody | (BI Clone I) | TPP-23298 | (Samsung) |
| TPP-30788 | | + | + |
| (BI Clone I) | | | |
| TPP-23298 | + | | − |
| TPP-17755 | + | − | |
| (Samsung) | | | |

“+” means injection of second antibody resulted in additional binding signal showing that the two tested antibodies bind to two different Sema3A epitopes
“−“ means injection of second antibody did not resulted in additional binding signal showing that the two tested antibodies compete for binding to overlapping or adjacent epitopes Sema3A epitopes

The binning experiment strongly points to another epitope for TPP-23298 compared to TPP-30788 (BI clone I) meaning that both antibodies target an independent/different epitope on Sema3A, whereas TPP-23298 might have overlapping or adjacent epitopes with TPP-17755 (Samsung).

### Example 7: Assessment of Binding to Off-Targets

To assess the specificity of an anti-Sema3A mAb (TPP-15370, parental mAb) an off-target screen using Retrogenix technology was conducted. For primary screening, 5484 expression vectors, encoding both ZsGreenl and a full-length human plasma membrane protein or a cell-surface tethered human secreted protein, were arrayed in duplicate across 16 microarray slides. Human HEK293 cells were used for reverse transfection/expression.

The test antibody was added to each slide after cell fixation giving a final concentration of 20 μg/ml. Detection of binding was performed by using the same AF647 anti-hIgG Fc detection antibody as used in the Pre-screen. Two replicate slides were screened for each of the 16 slide-sets. Hits were classified as ‘strong, medium, weak or very weak’, depending on the intensity of the duplicate spots.

Following a screen for binding against fixed HEK293 cells expressing 5484 human plasma membrane proteins and human secreted proteins, Retrogenix's technology identified no specific off-target interactions for test antibody TPP-15370. Binding to Sema3A—its primary target—was observed. These data indicate high specificity of TPP-15370 for its primary target.

### Example 8: Selectivity of Anti-Sema3A mAbs

Semaphorin proteins can be subdivided in five classes occurring in vertebrates (class 3-7). To assess the selectivity profile of parental anti-Sema3A mAbs TPP-15370 and TPP-15374 in the Semaphorin 3 class (Sema3A-G) an ELISA assay was conducted using Sema3A, Sema3B, Sema3C, Sema3D, Sema3E, and Sema3F molecules from R&D Systems. Both antibodies showed no binding to Sema3B, Sema3C, Sema3D, Sema3E and Sema3F.

Because Sema3G has been recently identified as kidney protective (PMID: 27180624), it was important to test whether the antibodies do not bind to Sema3G. For the assessment of binding selectivity to Sema3A vs Sema3G, 1 nM recombinant human Sema3A-Fc chimera (R&D Systems) or recombinant human GST—Sema3G (Abnova) were coated on Maxisorb plates, incubated with antibodies in a dose-response curve from 0.00015-10 μg/ml, and the binding of antibodies quantified using HRP coupled anti-human antiserum and chemiluminescent substrate.

TABLE 7
Off-target ELISA values for testing of Sema3G as off-target

| | EC50 [nM] | | Selectivity Score |
|---|---|---|---|
| | | Coating: | SEMA3G/ |
| Antibody | Coating: SEMA3A | SEMA3G | SEMA3A |
| TPP-23298 | 1.6 | >66667 | >41666 |
| TPP-23334 | 9.2 | >66667 | >7220 |
| TPP-23337 | 15.5 | >66667 | >4308 |
| TPP-23338 | 9.6 | >66667 | >6949 |
| TPP-23340 | 12.3 | >66667 | >5435 |
| TPP-23341 | 21.3 | >66667 | >3133 |
| TPP-23347 | 8.4 | >66667 | >7918 |
| TPP-23373 | 17.6 | >66667 | >3786 |
| TPP-23374 | 6.1 | >66667 | >10951 |
| TPP-23375 | 7.7 | >66667 | >8651 |
| TPP-11489 | Weak binding | >66667 | n.d. |
| (Chiome) | (EC50 not determinable) | | |
| TPP-17755 | Slight dose-response | >66667 | n.d. |
| (Samsung) | (not determinable) | | |
| TPP-30791 | 0.08 | >66667 | >833337 |
| (BI clone IV) | | | |
| TPP-30790 | 0.08 | >66667 | >833337 |
| (BI clone III) | | | |
| TPP-30789 | 0.10 | >66667 | >666670 |
| (BI clone II) | | | |
| TPP-30788 | 0.15 | >66667 | >444446 |
| (BI clone I) | | | |

All tested antibodies of the present disclosure as well as prior art antibodies do not bind to kidney protective Sema3G, as shown in Table 7.

Sema3A is a secreted protein that contains two furin cleavage sites and is present in an active an inactive cleaved form. In the in vivo situation Sema3A exists in both forms side by side. To test if anti-Sema3A antibodies are able to differentiate between the inactive and active form and to test how antibodies perform in binding to active Sema3A (resembled by full-length Sema3A (TPP-13211) in contrast to a inactive version as it only contains the Sema3A domain (resembled by cleaved Sema3A TPP-19068), an ELISA assay was performed. As readout out the ELISA signals of the tested antibody to the active Sema3A has been divided by the ELISA signals of the tested antibody to the inactive Sema3A.

TABLE 7a
Ratio for binding of anti-Sema3A antibodies to active vs. inactive
Sema3A as determined by ELISA

| | Ratio ELISA binding |
|---|---|
| Antibody | TPP-13211/TPP-19068* |
| TPPAntibody-23298 | 0.66 ± 0.14 |
| TPP-30788 (BI clone I) | 0.19 ± 0.03 |
| TPP-30789 (BI clone II) | 0.20 ± 0.07 |
| TPP-30790 (BI clone III) | 0.19 ± 0.03 |
| TPP-30791 (BI clone IV) | 0.21 ± 0.004 |

*A Ratio ELISA binding TPP-13211/TPP-19068 below 1 shows a higher binding activity to active Sema3A.
A Ratio ELISA binding TPP-13211/TPP-19068 above 1 shows a higher binding activity to inactive Sema3A.

The binding analysis as shown in Table 7a clearly showed that the antibody of the present disclosure (TPP-23298) shows increased binding to active Sema3A than TPP-30788-TPP-30791 (BI clones) presumably since they target a different epitope indicating a higher selectivity for active Sema3A.

### Example 9: In Vitro Efficacy in a Mesangial Cell Migration Assay

A confluent monolayer of human primary mesangial cells was generated by seeding cells in serum-containing culture medium into image lock plates for 24 hours. After switching to serum-free culture medium, scratch wounds were created using the WoundMaker tool, after which the cells were treated with 1 nM recombinant human Sema3A-Fc chimera (R&D Systems) in the absence or presence of inhibitory antibodies. The cells were imaged in the Incucyte and after 24 hrs the extent of wound closure was assessed using the Incucyte Integrated Cell Migration Analysis software module.

TABLE 8
EC50 values for phage display hits and recombination variants
in the MCM assay

| Antibody | EC50 [nM] |
|---|---|
| TPP-15051 (Chiome) | 42.87 |
| TPP-15354 | 31.87 |
| TPP-15355 | >200 |
| TPP-15356 | >200 |
| TPP-15357 | 158.13 |
| TPP-15358 | >200 |
| TPP-15359 | >200 |
| TPP-15360 | 37.47 |
| TPP-15361 | 118.67 |
| TPP-15362 | >200 |
| TPP-15363 | >200 |
| TPP-15364 | >200 |
| TPP-15365 | >200 |
| TPP-15366 | 2.27 |
| TPP-15367 | 148.07 |
| TPP-15368 | >200 |
| TPP-15369 | 45.47 |
| TPP-15370 | 4.13 |
| TPP-15371 | >200 |
| TPP-15372 | 86.87 |
| TPP-15373 | 123.53 |
| TPP-15374 | 5.07 |
| TPP-15375 | >200 |
| TPP-15376 | >200 |
| TPP-15377 | >200 |
| TPP-15378 | 67.00 |
| TPP-15379 | >200 |
| TPP-15380 | 125.53 |
| TPP-15381 | >200 |
| TPP-15382 | 199.87 |
| TPP-15384 | 1.60 |
| TPP-15385 | 1.20 |
| TPP-15386 | >200 |
| TPP-15387 | >200 |
| TPP-15388 | >200 |
| TPP-15389 | 103.60 |
| TPP-15390 | >200 |
| TPP-15391 | >200 |
| TPP-15392 | 62.53 |
| TPP-15393 | 131.93 |
| TPP-15394 | >200 |
| TPP-15395 | >200 |
| TPP-15396 | 82.67 |
| TPP-15397 | >200 |
| TPP-15398 | 6.00 |
| TPP-15399 | 197.13 |
| TPP-15400 | 4.73 |
| TPP-15401 | >200 |
| TPP-17755 (Samsung) | 11.33 |
| TPP-23298 | 0.40 |
| TPP-23334 | 0.67 |
| TPP-23337 | 0.33 |
| TPP-23338 | 0.60 |
| TPP-23340 | 0.87 |
| TPP-23341 | 0.90 |
| TPP-23345 | 0.93 |
| TPP-23346 | 1.27 |
| TPP-23347 | 0.67 |
| TPP-23373 | 0.63 |
| TPP-23374 | 0.30 |
| TPP-23375 | 1.03 |
| TPP-30788 (BI clone I) | 1.43 |

We identified antibodies with potencies in the three-digit picomolar range in the human Mesangial Cell Migration Assay, which is considerably more potent than the prior art antibodies, as shown in Table 8.

### Example 10: In Vitro Efficacy in a Growth Cone Collapse Assay

In the direction of determining the potency of the antibodies against Sema3A induced cytoskeletal collapse, a growth cone collapse assay was used similarly as described (PMID: 12077190) with a few modifications. In brief, mouse dorsal root ganglion (DRG) neurons were isolated from E13 C57Bl/6J mouse embryos, cultured on poly-L-lysine and laminin-coated 96-wells with Neurobasal medium+100 ng/mL NGF+B-27+10% FCS. After 20 hours, the cells were treated for 1 hour with 10 nM recombinant human Sema3A-Fc chimera (RnD Systems) in the absence or presence of inhibitory antibodies followed by PFA fixation and staining with Alexa488-phalloidin. The extent of growth cone collapse was assessed using immunofluorescence microscopy via actin growth cone area/shape/texture for more than 100 growth cones per well.

TABLE 9
EC50 values for phage display hits and recombination variants
in the GCC assay

| Antibody | EC50 (nM) |
|---|---|
| TPP-15051 (Chiome) | 243.40 |
| TPP-15354 | 67.73 |
| TPP-15355 | >200 |
| TPP-15356 | >200 |
| TPP-15357 | 50.73 |
| TPP-15358 | >200 |
| TPP-15359 | >200 |
| TPP-15360 | 31.07 |
| TPP-15361 | >200 |
| TPP-15362 | >200 |
| TPP-15363 | >200 |
| TPP-15364 | >200 |
| TPP-15365 | 142.87 |
| TPP-15366 | 4.13 |
| TPP-15367 | 170.87 |
| TPP-15368 | >200 |
| TPP-15369 | 76.60 |
| TPP-15370 | 4.33 |
| TPP-15371 | >200 |
| TPP-15372 | 109.47 |
| TPP-15373 | >200 |
| TPP-15374 | 8.13 |
| TPP-15375 | >200 |
| TPP-15376 | >200 |
| TPP-15377 | >200 |
| TPP-15378 | 138.60 |
| TPP-15379 | >200 |
| TPP-15380 | 135.40 |
| TPP-15381 | >200 |
| TPP-15382 | >200 |
| TPP-15384 | 18.80 |
| TPP-15385 | 6.00 |
| TPP-15386 | >200 |
| TPP-15387 | >200 |
| TPP-15388 | >200 |
| TPP-15389 | 160.67 |
| TPP-15390 | >200 |
| TPP-15391 | >200 |
| TPP-15392 | >200 |
| TPP-15393 | >200 |
| TPP-15394 | >200 |
| TPP-15395 | 66.47 |
| TPP-15396 | 180.80 |
| TPP-15397 | >200 |
| TPP-15398 | 12.00 |
| TPP-15399 | >200 |
| TPP-15400 | 25.73 |
| TPP-15401 | >200 |
| TPP17755 (Samsung) | 52.67 |
| TPP-23298 | 2.40 |
| TPP-23334 | 2.24 |
| TPP-23337 | 2.12 |
| TPP-23374 | 2.19 |

The identified antibodies also show potencies in the single digit nanomolar range in the murine Growth Cone Collapse Assay, again considerably more potent than the tested prior art antibodies (two- to three-digit nanomolar potency), as shown in Table 9.

### Example 11: In Vitro Efficacy in a HUVEC Repulsion Assay

Recombinant human Sema3A-Fc chimera (R&D Systems) is not identical to Sema3A in human biofluids because it contains several mutated amino acids and an extra protein fragment at its carboxy-terminus. Furthermore, the above described assays (human Mesangial Cell Migration Assay and murine Growth Cone Collapse Assay) use Sema3A in homogenous distribution, which is in contrast to the gradient distribution described for Sema3A in tissues. We hypothesized that these differences could result in a different potency of the antibodies towards recombinant versus endogenous protein. Therefore, we adapted an assay using a gradient of human wild-type Sema3A as agonist (PMID: 17569671). In brief, in this HUVEC repulsion assay, human embryonic kidney 293 cells (HEK293) cells expressing human Sema3A of the sequence of SEQ ID NO: 600, were seeded on a confluent monolayer of human umbilical vein endothelial cells (HUVEC) in EGM-2 medium in the absence or presence of inhibitory antibodies, cultured for 72 hours, fixed, stained with DAPI and the extent of cell repulsion assessed by immunofluorescence microscopy (measurement of cell free areas). Consequently, the substrate human Sema3A exists in excess.

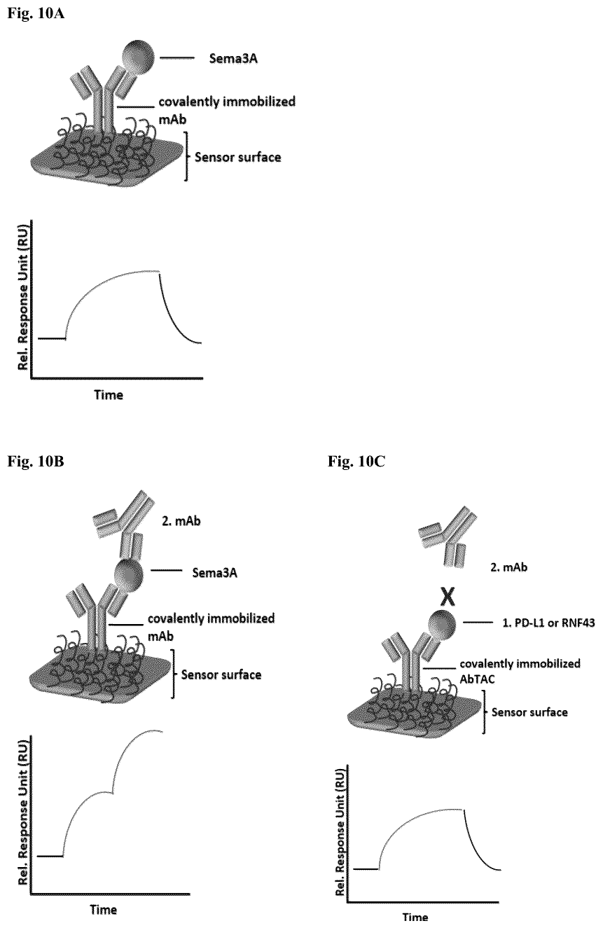

Based on immunofluorescence microscopy images of the DAPI/CM stained cells (CM=HCS CellMask™ Stain, stains the whole cell in order to define the total cell area) data analysis is performed as follows: Cells are identified based on the DAPI/CM signals (FIG. 11B). The cell area for analysis is defined and selected. In this area the cell-free region is calculated (FIG. 11C). Percent inhibition is calculated based on the “cell free-region” that is induced by Sema3A in the antibody-treated wells in comparison to the isotype-treated wells. Percent inhibition is plotted over antibody concentration and EC-50 values of the respective antibodies are calculated.

In detail the following steps are performed for the data analysis:
1. Four fields are imaged per well which corresponds to 80% of the well area. All of these fields stitched together are used for the detection of the cells via the DAPI/CM fluorescence.
2. The “cell area” is calculated based on the DAPI/CM area.
3. The “cell-free region” is calculated based on the “total area” subtracted by the “cell area”.
4. Percent inhibition is calculated based on the “cell free-region” that is induced by Sema3A in the antibody-treated wells vs the isotype-treated wells.
5. The software GraphPad Prism is used to determine the EC50 values using nonlinear regression (Variable slope model=four-parameter dose-response curve).

TABLE 10
EC50 values for selected antibodies in the repulsion assay,
first experiment

| Antibody | EC50 (pM) |
|---|---|
| TPP-15370 | 800 |
| TPP-23298 | 80 |
| TPP-23334 | 120 |
| TPP-23337 | 170 |
| TPP-23340 | 180 |
| TPP-23341 | 113 |
| TPP-23373 | 180 |
| TPP-23374 | 77 |
| TPP-23375 | 123 |

TABLE 10a
EC50 values for TPP-23298 in the repulsion assay in a second
experiment to compare to prior art antibodies

| Antibody | EC50 (pM) |
|---|---|
| TPP-23298 | 54 |
| TPP-30788 (BI clone I) | 104 |
| TPP-30789 (BI clone II) | 165 |
| TPP-30790 (BI clone III) | 121 |
| TPP-30791 (BI clone IV) | 221 |
| TPP-17755 (Samsung) | 2794 |
| TPP-11489 (Chiome) | >20000 |

The potency distinction to the prior art antibodies in the human Mesangial Cell Migration Assay and murine Growth Cone Collapse Assay above, is even more pronounced in this HUVEC Repulsion Assay that uses a gradient of native wt Sema3A (mixture of processed inactive and undigested active Sema3A) as shown in Table 10 and 10a. The improved potency in HUVEC repulsion assay in comparison to TPP-17755, to TPP-11489, to TPP-30788, to TPP-30789, TPP-30790, or to TPP-30791 is quantified measuring the picomolar activity as shown by the corresponding EC-50 values. While TPP-23298 shows two-digit picomolar activities, prior art antibody potencies of TPP-17755, TPP-11489, TPP-30788, TPP-30789, TPP-30790, or TPP-30791, are in the three-digit picomolar or even nanomolar range.

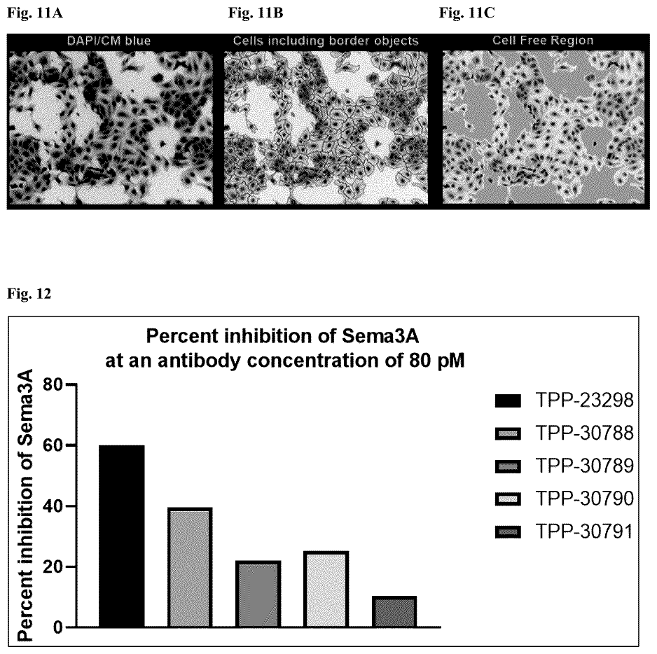

As an alternative illustration of the results, the improved potency in HUVEC repulsion assay is quantified by measuring the cell-free region at a specified concentration of 80 pM of the respective antibodies. TPP-23298 shows a higher percent inhibition of Sema3A than to TPP-30788, to TPP-30789, TPP-30790, or to TPP-30791 (FIG. 12).

Analyzing the data from both assays displayed in table 10 and 10a TPP-23298 shows the highest potency against cellular Sema3A induced HUVEC repulsion. The BI Antibodies TPP-30788, TPP-30798, TPP-30790 and TPP-30791 exhibited slightly higher EC50 values (2-5-fold). The Samsung Antibody TPP-17755 has a significantly lower potency than the TPP-23298 (50-fold). The Chiome Antibody TPP-11489 did only show inhibitory activity at the highest tested concentrations resulting in a predicted EC50 value>400-fold above antibody according to the present disclosure.

That shows that under conditions, that resembles a native environment without any spiked exogenous, recombinant semaphorin3A, the antibodies according to the present disclosure inhibit Sema3A-induced cell repulsion with the strongest activity, as shown in Table 10 and 10a.

### Example 12: In Vivo Assay for Detecting Protective Renal Effects: Inhibition of Sema3A-Induced Albuminuria in Mice

Sema3A inhibitors decrease urinary albumin excretion induced via systemic injection of recombinant Sema3A. The beneficial effect of the compounds on albuminuria reduction were investigated in a Sema3A-induced albuminuria model as follows:

Male C57Bl/6 mice (8- to 10-wk-old) purchased from Taconic were injected intravenously with anti-Sema3A antibodies. Thirty minutes after antibody application albuminuria was induced by intravenous injection of human recombinant Sema3A (1.0 mg/kg, R&D Systems). Animals were placed into metabolic cages and urine was collected for 4 h. Urinary creatinine was measured via clinical biochemistry analyzer (Pentra400). For the assessment of urinary albumin, a mouse specific Albumin ELISA (Abcam) was used according to manufacturer's protocol. Both urinary creatinine and albumin were used to calculate urinary albumin to creatine ratio (ACR). Differences between groups were analyzed by one-way ANOVA with Dunnett's corrections for multiple comparisons. Statistical significance is defined as p<0.05. All statistical analyses were done using GraphPad Prism 8.

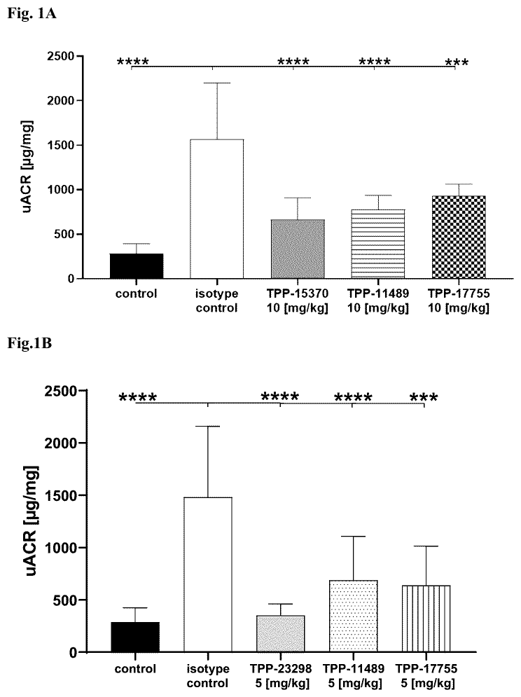

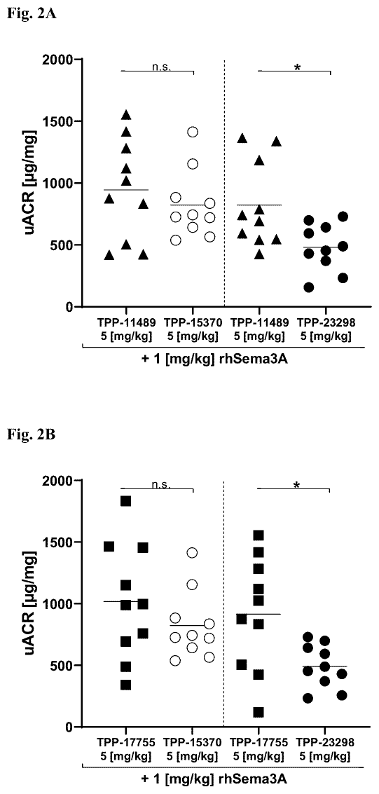

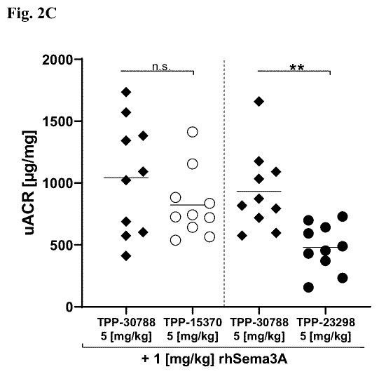

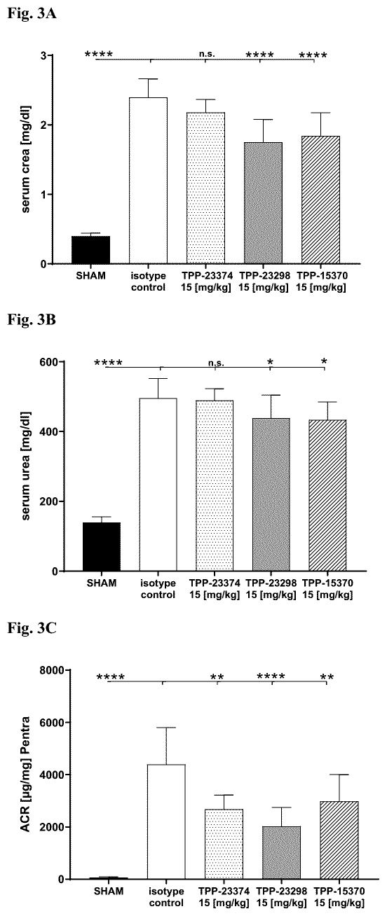

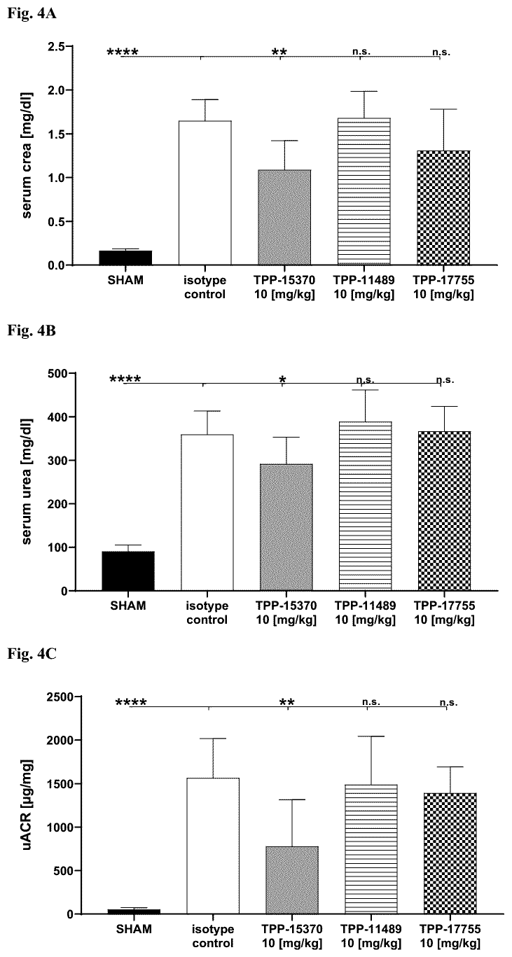

Table 11-15a show dose-response experiments with TPP-15370, TPP-15374, TPP-11489, TPP-17755, TPP-30788 and TPP-23298 in the Sema3A-induced albuminuria model in mice. Effects on albuminuria reduction with TPP-15370, TPP-23298 in comparison to TPP-11489 and/or TPP-17755 and/or TPP-30788 are shown in FIGS. 1A-2C.

The antibodies according to the present disclosure reduce Sema3A-induced urinary Albumin excretion.

TABLE 11
Dose-response of Sema3A-induced albuminuria reduction after
treatment with TPP-15370

| | urinary albumin | |
|---|---|---|
| | to creatinine ratio | |
| | [μg/mg] | |
| control; Mean ± SD | 345.30 ± | 102.15**** |
| 15 [mg/kg] isotype control; | 1392.80 ± | 350.70 |
| Mean ± SD | | |
| 1 [mg/kg] TPP-15370; Mean ± SD | 1030.80 ± | 216.27** |
| 5 [mg/kg] TPP-15370; Mean ± SD | 693.84 ± | 203.18**** |
| 15 [mg/kg] TPP-15370; Mean ± SD | 273.10 ± | 146.02**** |

8-10 animal/group, One-way ANOVA with Dunnett's corrections for multiple comparisons, */**/**/**** = significant with p < 0.05/0.01/0.001/0.0001 vs isotype control

TABLE 12
Dose-response of Sema3A-induced albuminuria reduction after
treatment with TPP-15374

| | urinary albumin | |
|---|---|---|
| | to creatinine ratio | |
| | [μg/mg] | |
| Control; Mean ± SD | 226.40 ± | 65.50**** |
| 15 [mg/kg] isotype control; | 1061.43 ± | 216.47 |
| Mean ± SD | | |
| 1 [mg/kg] TPP-15374; Mean ± SD | 782.60 ± | 122.43** |
| 5 [mg/kg] TPP-15374; Mean ± SD | 690.19 ± | 190.27**** |
| 15 [mg/kg] TPP-15374; Mean ± SD | 592.87 ± | 123.93**** |

8-10 animal/group, One-way ANOVA with Dunnett's corrections for multiple comparisons, */**/***/**** = significant with p < 0.05/0.01/0.001/0.0001 vs isotype control

TABLE 13
Dose-response of Sema3A-induced albuminuria reduction after
treatment with TPP-23298

| | urinary albumin | |
|---|---|---|
| | to creatinine ratio | |
| | [μg/mg] | |
| Control; Mean ± SD | 345.30 ± | 102.15**** |
| 15 [mg/kg] isotype control; | 1281.65 ± | 447.14 |
| Mean ± SD | | |
| 1 [mg/kg] TPP-23298; Mean ± SD | 623.37 ± | 240.41**** |
| 5 [mg/kg] TPP-23298; Mean ± SD | 471.07 ± | 164.97**** |
| 15 [mg/kg] TPP-23298; Mean ± SD | 320.60 ± | 166.36**** |

8-10 animal/group, One-way ANOVA with Dunnett's corrections for multiple comparisons,*/**/***/**** = significant with p < 0.05/0.01/0.001/0.0001 vs isotype control

TABLE 14
Dose-response of Sema3A-induced albuminuria reduction after
treatment with TPP-11489

| | urinary albumin | |
|---|---|---|
| | to creatinine ratio | |
| | [μg/mg] | |
| Control; Mean ± SD | 237.23 ± | 92.61**** |
| 15 [mg/kg] isotype control; | 1404.81 ± | 411.55 |
| Mean ± SD | | |
| 1 [mg/kg] TPP-11489; Mean ± SD | 1204.81 ± | 426.64 |
| 5 [mg/kg] TPP-11489; Mean ± SD | 664.02 ± | 228.96**** |
| 15 [mg/kg] TPP-11489; Mean ± SD | 572.42 ± | 211.05**** |

8-10 animal/group, One-way ANOVA with Dunnett's corrections for multiple comparisons, */**/***/**** = significant with p < 0.05/0.01/0.001/0.0001 vs isotype control

TABLE 15
Dose-response of Sema3A-induced albuminuria reduction after
treatment with TPP-17755

| | urinary albumin | |
|---|---|---|
| | to creatinine ratio | |
| | [μg/mg] | |
| Control; Mean ± SD | 298.02 ± | 91.06**** |
| 15 [mg/kg] isotype control; | 1053.75 ± | 162.28 |
| Mean ± SD | | |
| 1 [mg/kg] TPP-17755; Mean ± SD | 932.57 ± | 221.09 |
| 5 [mg/kg] TPP-17755; Mean ± SD | 823.11 ± | 196.93* |
| 15 [mg/kg] TPP-17755; Mean ± SD | 711.09 ± | 181.65*** |

8-10 animal/group, One-way ANOVA with Dunnett's corrections for multiple comparisons, */**/***/**** = significant with p < 0.05/0.01/0.001/0.0001 vs isotype control

TABLE 15a
Dose-response of Sema3A-induced
albuminuria reduction after treatment with TPP-30788

| | urinary albumin to |
|---|---|
| | creatinine ratio [μg/mg] |
| Control; Mean ± SD | 266.67 ± 115.66**** |
| 15 [mg/kg] isotype control; Mean ± SD | 1546.59 ± 312.43 |
| 1 [mg/kg] TPP-30788; Mean ± SD | 1234.13 ± 353.48 |
| 5 [mg/kg] TPP-30788; Mean ± SD | 958.30 ± 196.93*** |
| 15 [mg/kg] TPP-30788; Mean ± SD | 841.46 ± 438.51**** |

8-10 animal/group, One-way ANOVA with Dunnett's corrections for multiple comparisons,
*/**/***/**** = significant with p < 0.05/0.01/0.001/0.0001 vs isotype control

### Example 13: In Vivo Assay for Detecting Protective Renal Effects: Acute Ischemia/Reperfusion Injury (I/RI) Model in Mice

Unilaterally nephrectomized mice may benefit from treatment with Sema3A inhibitors after ischemia reperfusion injury. The beneficial effect of Sema3A antibodies on kidney function was investigated in a kidney ischemia-reperfusion injury model in mice as follows:

Laboratory bred male C57Bl/6J mice 6-8 weeks old were obtained from Charles River. Mice were maintained under standard laboratory conditions, 12-hour light-dark cycles with access to normal chow and drinking water at libitum. For the ischemia reperfusion injury model, a total of 8-10 was used in each control and experimental group.

Animals were anesthetized with continuous inhaled isoflurane. Right nephrectomy was performed through a right flank incision 7 days before the ischemic procedures in the contralateral kidneys. One-hour before the initiation of renal ischemia antibodies and adequate isotype control were administrated to mice via i.v. injection. Mice were anesthetized and a left flank incision was made. Renal vessels were exposed by dissection of the left renal pedicle. Non-traumatic vascular clamps were used to stop blood flow (artery and vein) during 25 min (mice) of ischemia. Reperfusion was established by removing the clamps. The abdominal wall (muscular layer and skin) was closed with 5.0 polypropylene sutures. Temgesic® (Buprenorphin, 0.025 mg/kg s.c.) was applied as an analgesic.

Urine of each animal was collected in metabolic cages over night before sacrifice at 24 h post ischemia. Urinary creatinine was measured by a clinical biochemistry analyzer (Pentra400). For the assessment of urinary albumin, a mouse specific Albumin Kit (Hitachi) was used within the Pentra analyzer. Both urinary creatinine and albumin were used to determine Albuminuria (albumin/creatinine ratio). Upon sacrifice, blood samples were obtained under terminal anesthesia. After centrifugation of the blood samples, serum was isolated. Both serum creatinine and serum urea were measured via clinical biochemistry analyzer (Pentra 400). Differences between groups were analyzed by one-way ANOVA with Dunnett's corrections for multiple comparisons. Statistical significance is defined as p<0.05. All statistical analyses were done using GraphPad Prism 8.

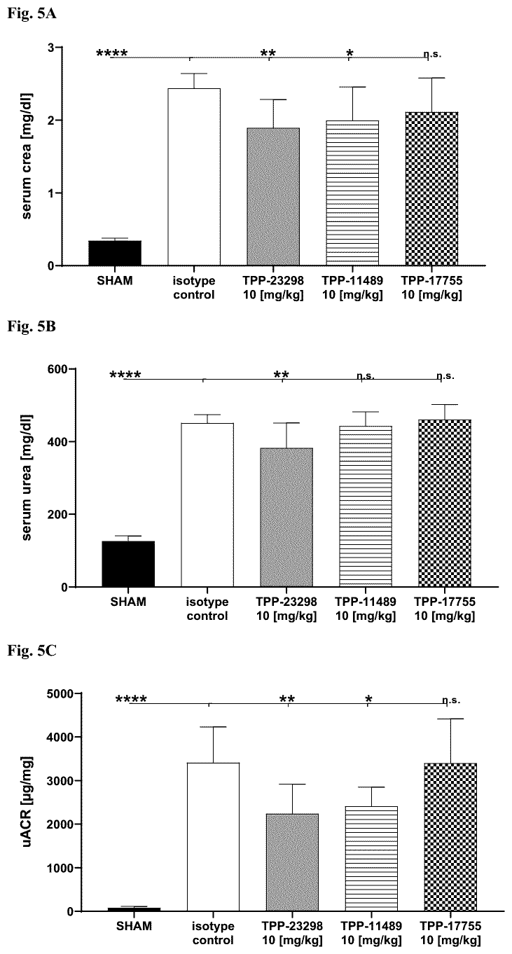

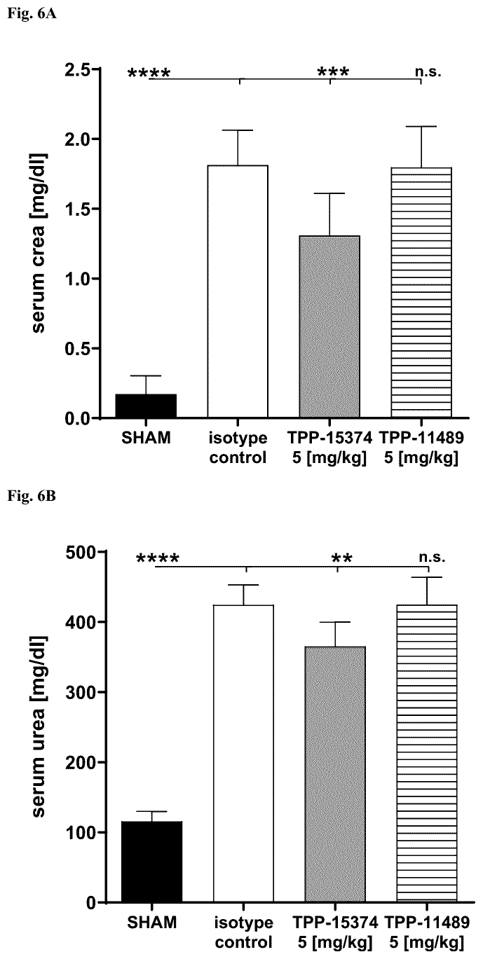

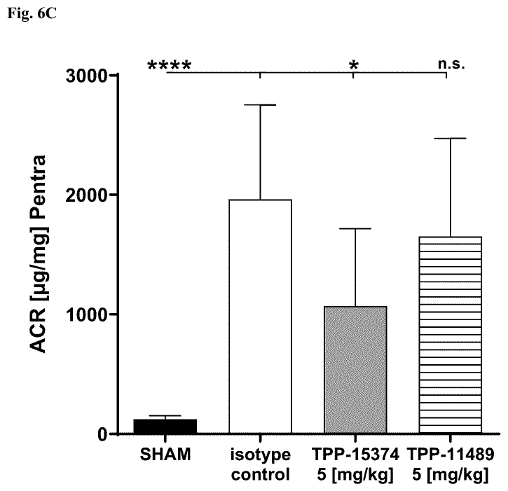

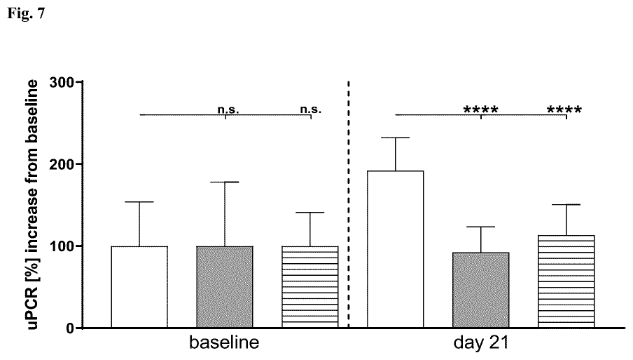

Table 16-20 show dose-response experiments with TPP-15370, TPP-15374, TPP-11489, TPP-17755 and TPP-23298 in an acute renal ischemia/reperfusion injury model in mice. FIGS. 3A-3C show the efficacy of TPP-23374, TPP-23298 and TPP-15370 after treatment with 15 mg/kg in the I/RI model. Treatment effects with TPP-15370, TPP-23298 and TPP-15374 in comparison to TPP-11489 and/or TPP-17755 are shown in FIGS. 4A-6C.

The antibodies attenuated ischemia/reperfusion induced kidney damage by reducing serum creatinine and serum urea (surrogates for glomerular filtration rate) and excretion of urinary albumin.

TABLE 16
Dose-response of TPP-15370 in mouse I/R injury model

| | serum | | urinary albumin |
|---|---|---|---|
| | creatinine | serum urea | to creatinine |
| | [mg/dl] | [mg/dl] | ratio [μg/mg] |
| SHAM | 0.34 ± 0.05**** | 102.78 ± 9.45**** | 58.50 ± 19.22**** |
| Mean ± SD | | | |
| 15 [mg/kg] | 1.72 ± 0.30 | 385.63 ± 41.69 | 1699.47 ± 461.60 |
| isotype | | | |
| control | | | |
| Mean ± SD | | | |
| 1 [mg/kg] | 1.61 ± 0.52 | 396.51 ± 86.91 | 1165.37 ± 445.50** |
| TPP-15370 | | | |
| Mean ± SD | | | |
| 5 [mg/kg] | 1.22 ± 0.32* | 297.92 ± 70.02** | 705.21 ± 192.26** |
| TPP-15370 | | | |
| Mean ± SD | | | |
| 15 [mg/kg] | 0.89 ± 0.27**** | 261.95 ± 27.76*** | 554.52 ± 133.99**** |
| TPP-15370 | | | |
| Mean ± SD | | | |

8-10 animal/group, One-way ANOVA with Dunnett's corrections for multiple comparisons,
*/**/***/**** = significant with p < 0.05/0.01/0.001/0.0001 vs isotype control

TABLE 17
Dose-response of TPP-15374 in mouse I/R injury model

| | serum | serum | urinary albumin |
|---|---|---|---|
| | creatinine | urea | to creatinine |
| | [mg/dl] | [mg/dl] | ratio [μg/mg] |
| SHAM | 0.26 ± 0.02**** | 113.90 ± 29.95**** | 39.36 ± 10.19**** |
| Mean ± SD | | | |
| 15 [mg/kg] | 2.09 ± 0.19 | 494.52 ± 29.75 | 3942.50 ± 1790.29 |
| isotype | | | |
| control | | | |
| Mean ± SD | | | |
| 1 [mg/kg] | 1.84 ± 0.39 | 478.10 ± 66.55 | 2774.43 ± 946.18 |
| TPP-15374 | | | |
| Mean ± SD | | | |
| 5 [mg/kg] | 1.66 ± 0.32* | 416.49 ± 98.47* | 2195.95 ± 900.56* |
| TPP-15374 | | | |
| Mean ± SD | | | |
| 15 [mg/kg] | 1.43 ± 0.34**** | 389.02 ± 5128** | 1495.88 ± 560.06** |
| TPP-15374 | | | |
| Mean ± SD | | | |

8-10 animal/group, One-way ANOVA with Dunnett's corrections for multiple comparisons,
*/**/***/**** = significant with p < 0.05/0.01/0.001/0.0001 vs isotype control

TABLE 18
Dose-response of TPP-11489 in mouse I/R injury model

| | serum | serum | urinary albumin |
|---|---|---|---|
| | creatinine | urea | to creatinine |
| | [mg/dl] | [mg/dl] | ratio [μg/mg] |
| SHAM | 0.22 ± 0.02**** | 57.64 ± 14.62**** | 27.87 ± 13.55**** |
| Mean ± SD | | | |
| 15 [mg/kg] | 1.99 ± 0.29 | 410.18 ± 39.80 | 1569.47 ± 277.70 |
| isotype | | | |
| control | | | |
| Mean ± SD | | | |
| 1 [mg/kg] | 2.00 ± 0.12 | 453.84 ± 26.54 | 1600.96 ± 338.48 |
| TPP-11489 | | | |
| Mean ± SD | | | |
| 5 [mg/kg] | 1.92 ± 0.16 | 416.87 ± 49.81 | 1437.08 ± 323.46 |
| TPP-11489 | | | |
| Mean ± SD | | | |
| 15 [mg/kg] | 1.68 ± 0.42* | 367.67 ± 39.32 | 1186.32 ± 366.49* |
| TPP-11489 | | | |
| Mean ± SD | | | |

8-10 animal/group, One-way ANOVA with Dunnett's corrections for multiple comparisons,
**/***/**** = significant with p < 0.05/0.01/0.001/0.0001 vs isotype control

TABLE 19
Dose-response of TPP-17755
antibody in mouse I/R injury model

| | serum | | urinary albumin |
|---|---|---|---|
| | creatinine | serum urea | to creatinine |
| | [mg/dl] | [mg/dl] | ratio [μg/mg] |
| SHAM | 0.21 ± 0.06**** | 91.20 ± 34.20**** | 75.45 ± 42.78**** |
| Mean ± SD | | | |
| 15 [mg/kg] | 1.75 ± 0.30 | 444.25 ± 64.25 | 1791.23 ± 543.46 |
| isotype | | | |
| control | | | |
| Mean ± SD | | | |
| 1 [mg/kg] | 1.74 ± 0.27 | 430.30 ± 75.96 | 1659.08 ± 577.99 |
| TPP-17755 | | | |
| Mean ± SD | | | |
| 5 [mg/kg] | 1.84 ± 0.24 | 439.83 ± 73.68 | 1661.14 ± 460.41 |
| TPP-17755 | | | |
| Mean ± SD | | | |
| 15 [mg/kg] | 1.31 ± 0.37** | 346.62 ± 78.14** | 1351.64 ± 795.59 |
| TPP-17755 | | | |
| Mean ± SD | | | |

8-10 animal/group, One-way ANOVA with Dunnett's corrections for multiple comparisons,
*/**/***/**** = significant with p < 0.05/0.01/0.001/0.0001 vs isotype control

TABLE 20
Dose-response of TPP-23298 antibody
in mouse I/R injury model

| | serum | serum | urinary albumin |
|---|---|---|---|
| | creatinine | urea | to creatinine |
| | [mg/dl] | [mg/dl] | ratio [μg/mg] |
| SHAM | 0.26 ± 0.04**** | 115.80 ± 6.76**** | 71.05 ± 865.39**** |
| Mean ± SD | | | |
| 15 [mg/kg] | 2.53 ± 0.15 | 498.92 ± 45.45 | 3968.71 ± 453.52 |
| isotype | | | |
| control | | | |
| Mean ± SD | | | |
| 1 [mg/kg] | 2.38 ± 0.22 | 482.06 ± 25.84 | 2383.77 ± 1111.94** |
| TPP-23298 | | | |
| Mean ± SD | | | |
| 5 [mg/kg] | 2.20 ± 0.36* | 425.64 ± 58.85* | 1966.11 ± 677.69**** |
| TPP-23298 | | | |
| Mean ± SD | | | |
| 15 [mg/kg] | 2.02 ± 0.28*** | 422.79 ± 71.44** | 1949.56 ± 700.58**** |
| TPP-23298 | | | |
| Mean ± SD | | | |

8-10 animal/group, One-way ANOVA with Dunnett's corrections for multiple comparisons. One-way ANOVA with Dunnett's corrections for multiple comparisons,
*/**/***/**** = significant with p < 0.05/0.01/0.001/0.0001 vs isotype control

### Example 14: In Vivo Assay for Detecting Protective Renal Effects: Alport Syndrome Model (Col4α3 Deficient Mice)

The phenotype of Alport mice is similar to that of Alport patients, including characteristic thickening and splitting of the glomerular basement membrane as well as strong proteinuria. Alport mice may benefit from treatment with Sema3A inhibitors due to increased Sema3A expression in kidneys of those mice. The beneficial effect of Sema3A blocking antibodies on kidney function was investigated in the Alport mouse model as follows: A colony of knockout Col4α3 (129-Col4a3<tm1Dec>/J) mice (Jackson Laboratory, USA) was established by mating heterozygous animals within the breeding facilities at Bayer AG, Wuppertal, Germany. Male and female homozygous and wild-type Col4α3 mice at an age of 4-5 weeks were obtained from the animal breeding facilities at Bayer AG and used for this study.

The homozygous mice (HOM) were randomized into groups (n=10 each group) according to their age and gender. Mice were dosed once weekly with isotype control and TPP-15370 and TPP-23298. TPP-11489 was administered biweekly. Urine of each animal was collected in metabolic cages once weekly starting before initiation of treatment. Urinary creatinine as well as total protein was measured by a clinical biochemistry analyzer (Pentra400). Both urinary creatinine and albumin were used to determine proteinuria (protein/creatinine ratio). Upon sacrifice at day 21 or day 28 post treatment start, blood samples were obtained under terminal anesthesia. After centrifugation of the blood samples, serum was isolated. Both serum creatinine and serum urea were measured via clinical biochemistry analyzer (Pentra 400).

Kidneys were collected and divided in two parts. One part was snap-frozen in liquid nitrogen for mRNA analysis. The other part was stored in Davidson's fixative for the preparation of histological sections. Total RNA was isolated from parts of harvested kidneys. Kidney tissue was homogenized, and RNA was obtained and transcribed to cDNA. Using TaqMan real time PCR renal mRNA expression of pro-fibrotic markers was analyzed in kidney tissues. For the assessment of fibrosis on the protein level paraffin tissue sections were stained with alpha-smooth muscle actin (aSMA) and Sirius Red/Fast Green Collagen staining using standard procedures.

Quantitative measurements of alpha-smooth muscle actin (αSMA)-positive as well as Sirius Red (Collagen) positive areas within the kidneys were obtained by computer image analysis using the Axio Scan Z1 (Zeiss) microscope and the Zen software.

All data are expressed as means±S.D. Differences between groups were analyzed by one-way ANOVA with Dunnett's corrections for multiple comparisons. Statistical significance was defined as p<0.05. All statistical analyses were done using GraphPad Prism 8.

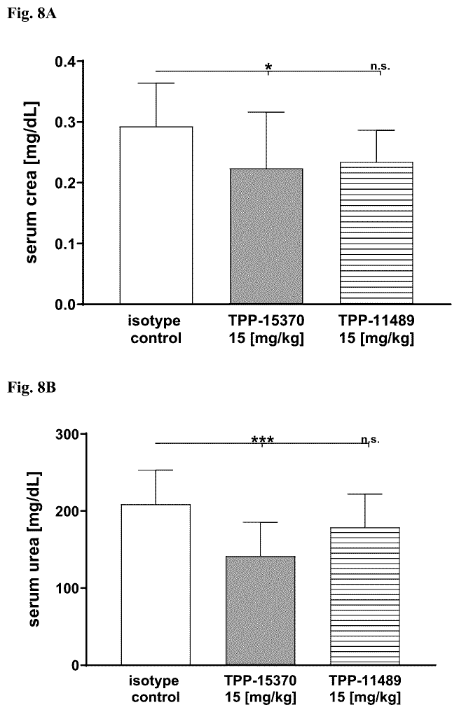

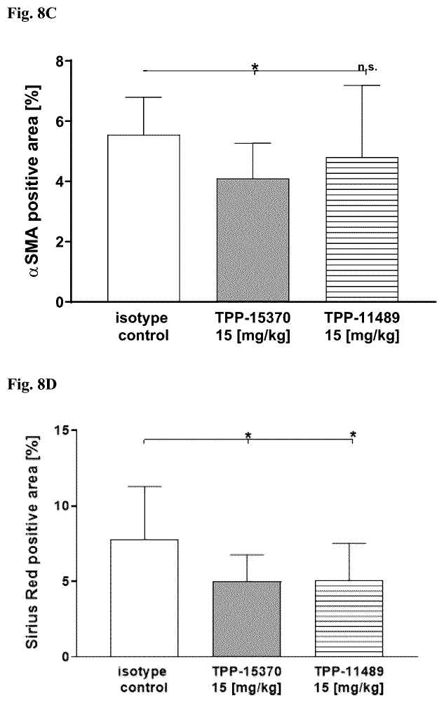

Tables 21A-21C and 22A-22C show effects on proteinuria, kidney function and kidney fibrosis obtained after treatment with TPP-15370 and TPP-23298 in the Alport model. Effects after treatment with TPP-15370 in comparison to TPP-11489 on proteinuria, kidney function and kidney fibrosis are displayed in FIGS. 7 and 8A-8D.

The antibodies according to the present disclosure stopped the progression of kidney disease in a mouse model of Alport syndrome. The antibodies according to the present disclosure reduced the excretion of urinary protein, reduced creatinine and serum urea (surrogates for glomerular filtration rate) as well as fibrosis quantified via myofibroblasts staining and collagen deposition.

TABLE 21A
Effects of TPP-15370 on proteinuria
progression in Alport mouse model

| | urinary protein to creatinine ratio | | | |
|---|---|---|---|---|
| | [%] increase from baseline | | | |
| | baseline | day 7 | day 14 | day 21 |
| HOM | 100.00 ± 53.71 | 118.65 ± 47.18 | 167.49 ± 55.77 | 192.03 ± 40.23 |
| 15 [mg/kg] | | | | |
| isotype | | | | |
| control | | | | |
| Mean ± SD | | | | |
| HOM | 100.00 ± 54.02 | 114.61 ± 50.48 | 149.35 ± 95.41 | 164.92 ± 47.18 |
| 5 [mg/kg] | | | | |
| TPP-15370 | | | | |
| Mean ± SD | | | | |
| HOM | 100.00 ± 65.59 | 114.61 ± 50.48 | 95.41 ± 52.50** | 93.04 ± 31.26**** |
| 15 [mg/kg] | | | | |
| TPP-15370 | | | | |
| Mean ± SD | | | | |

10 animal/group, data are expressed as relative means ± SD percentage values calculated vs. baseline (set to 100). Differences between groups were analyzed by one-way ANOVA with Dunnett's corrections formultiple comparisons. Statistical significance was defined as p ≤ 0.05.

TABLE 21B
Effects of TPP-15370 on functional
parameters at day 21 in Alport mouse model

| | serum creatinine | serum urea |
|---|---|---|
| | [mg/dl] | [mg/dl] |
| HOM | 0.71 ± 0.26 | 380.61 ± 120.28 |
| 15 [mg/kg] | | |
| isotype control | | |
| Mean ± SD | | |
| HOM | 0.39 ± 0.16** | 255.25 ± 56.80** |
| 5 [mg/kg] | | |
| TPP-15370 | | |
| Mean ± SD | | |
| HOM | 0.44 ± 0.21** | 256.71 ± 95.03** |
| 15 [mg/kg] | | |
| TPP-15370 | | |
| Mean ± SD | | |

10-15 animal/group, One-way ANOVA with Dunnett's corrections for multiple comparisons. One-way ANOVA with Dunnett's corrections for multiple comparisons,
**/***/**** = significant with p < 0.05/0.01/0.001/0.0001 vs isotype control

TABLE 21C
Effects of TPP-15370 on
fibrosis at day 28 in Alport mouse model

| | Myofibroblasts | Collagen |
|---|---|---|
| | % αSMA reduction | % Sirius Red reduction |
| HOM | 100.00 ± 53.53 | 100.00 ± 47.78 |
| 15 [mg/kg] | | |
| isotype control | | |
| Mean ± SD | | |
| HOM | 50.18 ± 21.00** | 80.08 ± 51.58 |
| 5 [mg/kg] | | |
| TPP-15370 | | |
| Mean ± SD | | |
| HOM | 54.86 ± 17.60** | 100.26 ± 50.97 |
| 15 [mg/kg] | | |
| TPP-15370 | | |
| Mean ± SD | | |

10-15 animal/group, One-way ANOVA with Dunnett's corrections for multiple comparisons. One-way ANOVA with Dunnett's corrections for multiple comparisons,
**/***/**** = significant with p < 0.05/0.01/0.001/0.0001 vs isotype control

TABLE 22A
Effects of TPP-23298 on proteinuria
progression in Alport mouse model

| | urinary protein to creatinine ratio | | | |
|---|---|---|---|---|
| | [%] increase from baseline | | | |
| | baseline | day 14 | day 21 | day 28 |
| HOM | 100.00 ± 70.94 | 185.29 ± 88.09 | 228.62 ± 160.68 | 283.62 ± 77.37 |
| 15 [mg/kg] | | | | |
| isotype | | | | |
| control | | | | |
| Mean ± SD | | | | |
| HOM | 100.00 ± 55.72 | 148.01 ± 77.13 | 155.25 ± 61.60 | 151.82 ± 45.84**** |
| 5 [mg/kg] | | | | |
| TPP-23298 | | | | |
| Mean ± SD | | | | |
| HOM | 100.00 ± 56.02 | 154.58 ± 91.21 | 120.54 ± 37.21**** | 125.71 ± 34.25**** |
| 15 [mg/kg] | | | | |
| TPP-23298 | | | | |
| Mean ± SD | | | | |

10 animal/group, data are expressed as relative means ± SD percentage values calculated vs. baseline (set to 100). Differences between groups were analyzed by one-way ANOVA with Dunnett's corrections for multiple comparisons. Statistical significance was defined as p ≤ 0.05.

TABLE 22B
Effects of TPP-23298 on functional
parameters at day 28 in Alport mouse model

| | serum creatinine | serum urea |
|---|---|---|
| | [mg/dl] | [mg/dl] |
| HOM | 0.29 ± 0.07 | 208.89 ± 0.07 |
| 15 [mg/kg] | | |
| isotype control | | |
| Mean ± SD | | |
| HOM | 0.22 ± 0.09* | 175.54 ± 0.03 |
| 5 [mg/kg] | | |
| TPP-23298 | | |
| Mean ± SD | | |
| HOM | 0.19 ± 0.03*** | 141.84 ± 0.03*** |
| 15 [mg/kg] | | |
| TPP-23298 | | |
| Mean ± SD | | |

10-15 animal/group, One-way ANOVA with Dunnett's corrections for multiple comparisons. One-way ANOVA with Dunnett's corrections for multiple comparisons,
**/***/**** = significant with p < 0.05/0.01/0.001/0.0001 vs isotype control

TABLE 22C
Effects of TPP-23298 on fibrosis
at day 28 in Alport mouse model

| | Myofibroblasts | Collagen |
|---|---|---|
| | % αSMA positive area | % Sirius Red positive area |
| HOM | 100.00 ± 53.53 | 100.00 ± 47.78 |
| 15 [mg/kg] | | |
| isotype control | | |
| Mean ± SD | | |
| HOM | 50.18 ± 21.00** | 80.08 ± 51.58 |
| 5 [mg/kg] | | |
| TPP-23298 | | |
| Mean ± SD | | |
| HOM | 54.86 ± 17.60** | 100.26 ± 50.97 |
| 15 [mg/kg] | | |
| TPP-23298 | | |
| Mean ± SD | | |

10-15 animal/group, One-way ANOVA with Dunnett's corrections for multiple comparisons. One-way ANOVA with Dunnett's corrections for multiple comparisons,
**/***/**** = significant with p < 0.05/0.01/0.001/0.0001 vs isotype control

### Example 15: In Vivo Assay for Detecting Protective Renal Effects: Unilateral Kidney IRI Model in Pigs

TPP-23298 was tested in a minimal invasive, unilateral kidney artery balloon-catheter occlusion model in adult minipigs with a post-reperfusion follow-up of about 24 hours. Female Gottingen mini pigs of a body weight range 14 to 17 kg (Ellegaard, Denmark) were used for the experiments. Animals were randomly assigned to experimental groups.

TPP-23298 was administered in a blinded, controlled study to 6 animals in comparison to 6 matched IgG-treated controls. Animals which were subjected to all treatment procedures without kidney artery occlusion and received phosphate buffered saline vehicle only served as sham treated reference group.

TPP-23298 was administered at weight adjusted doses in a final volume of 1 ml/kg phosphate buffered saline as a bolus by slow intravenous injection before start of kidney artery occlusion (preventive setting).

For the intervention on day 1 of experimentation pigs were anesthetized by a combination of Propofol and Fentanyl and artificially ventilated over an oro-tracheal tube under muscular relaxation by Pancuronium. Volume was continuously substituted by continuous infusion of Ringer lactate solution. Before starting surgery, antibiotic and thrombosis prophylaxis were provided by administration of Enrofloxacin i.m. and Heparin i.v., respectively. Blood pressure and heart rate were monitored with a non-invasive veterinary device equipped with a foreleg cuff.

All following interventions were performed under strictly aseptic conditions. A catheter was tunneled subcutaneously through the dorsal neck skin to a jugular vein for drug administration. A sheath was placed into the—preferably left—femoral artery and fixed, through which a hockey-stick catheter with a balloon catheter inside was advanced upstream into the abdominal aorta and inserted with its tip into the orifice of the left or right kidney artery. The balloon catheter was then protruded, and the balloon was inflated to interrupt blood flow to the kidney. Correct positioning of the balloon was controlled by Doppler ultrasound using a commercial ultrasound diagnostic apparatus. Plasma samples were collected at baseline and 2 h after start of ischemia.

Kidney ischemia was relieved exactly at pre-defined time points after start of occlusion (ranging from 90 to 120 min) by deflating the balloon and withdrawing the catheter and the sheath. After vascular suture and wound closure animals were re-awakened from anesthesia and after onset of spontaneous breathing extubated.

About 22 to 23 hours after the kidney artery occlusion animals were re-anesthetized by a combination of Ketaset/Dormicum and Pancuronium and artificially ventilated as described. Blood pressure and heart rate were invasively monitored via a carotid artery catheter. Volume substitution was provided at a flow rate of 10 ml/kg/h Ringer Lactate intravenously. Via a small incision in the lower abdomen both ureters were dissected on the urinary bladder wall and catheters were inserted to collect urine side separately for volume determination and urinalysis. Recordings and sample collections were started when all parameters were stable, which was typically the case 24 hours after occlusion. Blood samples were collected at baseline and every hour for three hours (24-27 h interval). In parallel urine was collected for three intervals of 1 h.

After urine volume flow (V_{U}) and urinary creatinine concentrations ([Crea]_{U}) were determined creatinine clearance (CL_{Crea}) was calculated side separately according to the standard formula CL_{Crea}=V_{U}*[Crea]_{U}/[Crea]_{Pl} in which [Crea]_{Pl} stands for plasma creatinine concentration. Global CL_{Crea} was calculated by adding CL_{Crea} of left and right kidney of each animal.

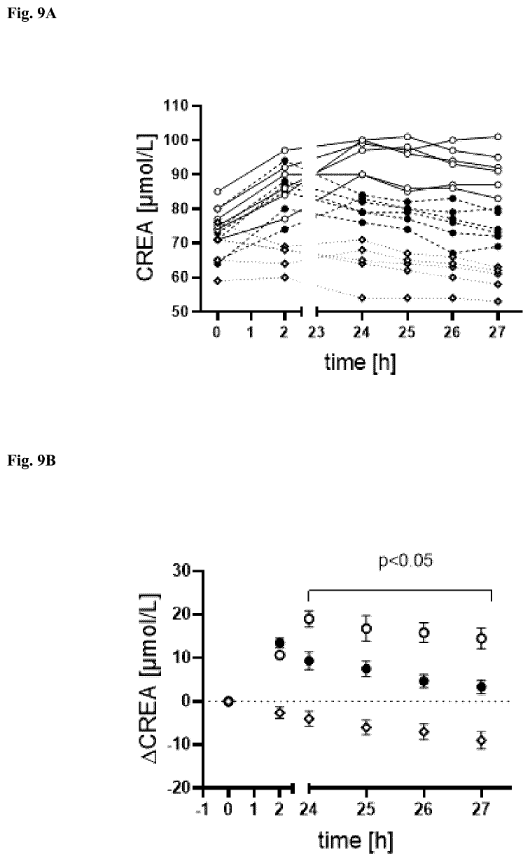

The results are depicted in FIGS. 9A-9D. TPP-23298 when administered in a preventive manner 30 min before occlusion prevented deterioration of ischemia/reperfusion-induced creatinine clearance significantly in this experimental setting after a unilateral kidney artery occlusion of 105 min.

### Example 16: Expression Titer of Anti-Sema3A Antibodies in Mammalian Cell Culture

HEK293-6E cells were transfected with pTT5 plasmids coding for the heavy and light chain of anti-Sema3A antibodies or with the Fab fragment of TPP-30792 (TPP-31357). Two days prior to transfection, HEK293-6E cells were split to a density of 5×10\hat{ }5 cells/mL in FreeStyle™ F17 Expression Medium (Gibco, A1383501) with 0.1% Pluronic F68 (Gibco, 24040032) and 4 mM GlutaMax (Gibco, 35050061) in a shake flask, making up 90% of the desired expression volume. HEK293-6E cells were cultivated at 37° C., 5% CO₂ shaking at 75 rpm.

For transfection, the DNA and polyethylenimine (Polysciences, 29366) are mixed in FreeStyle™ F17 Expression Medium (Gibco, A1383501) with 4 mM GlutaMax (Gibco, 35050061) making up 10% of the final expression volume. The solution is incubated for 10 minutes and added to the shake flask.

24 hours after transfection, 1% (v/v) ultra-low IgG FBS (Gibco, 16250078) and 0.05% (v/v) iN valproic acid (Sigma, P4543) are added to the shake flask.

The cell viability and density are monitored every day starting 4 days post transfection, the supernatant is harvested by centrifugation and sterile filtration when the viability is determined to be 70%. To determine the production titer, 100 μL of the harvested supernatant are loaded to a 0.1 mL Poros A affinity column (Thermo Scientific, 2100100) via HPLC-system (Agilent, 1100 HPLC system) using 50 mM sodium phosphate (Sigma, S0751, S9763), 150 mM NaCl (Sigma, S6546), 5% 2-propanol (sigma, 34863), pH 7.2 as running buffer. Subsequently, the protein is eluted using 12 mM HCl (Sigma, H9892), 150 mM NaCl, 5% 2-propanol pH 2. A calibration curve from 5 μg/mL to 150 μg/mL is set up using a protein of known size and is applied to the Poros A column via HPLC-system as well. Taking the size and extinction coefficient of the protein in the supernatant into consideration, the exact titer can be calculated using the standard curve. Expression in CHO is similar to HEK cells except that plasmid pTT22AKT was used for TPP-30792.

TABLE 23
Expression Titer of anti-Sema3A
antibodies in mammalian cells in mg/L

| | Titer [mg/L] |
|---|---|
| TPP-23298 | 203.6 |
| TPP-17755 (Samsung) | 277.0 |
| TPP-11489 (Chiome) | 132.0 |
| TPP-30791 (BI clone IV) | 333.0 |
| TPP-30790 (BI clone III) | 160.9 |
| TPP-30789 (BI clone II) | 187.6 |
| TPP-30788 (BI clone I) | 240.2 |
| TPP-30792 (3H4 Univ Ramot) | 3.0 (HEK), 3.2 (CHO) |
| TPP-31357 (Fab of TPP-30792) | Not determined |

The antibody of the present disclosure as well as all prior art antibodies except TPP-30792 can be produced with high titers in mammalian cells, as shown in Table 23. TPP-30792 could not be expressed in a significant amount in either HEK or CHO cells. In total 125 μg of TPP-30792 could be purified out of 4.5 liters of HEK293 cell culture. Similarly, the Fab fragment of TPP30792 (TPP-31357) yielded only 200 μg purified Fab out of 5 liters HEK293 cell culture.

### Example 17: Analysis of CMC Parameter Stability and Solubility of Anti-Sema3A Antibodies

It is known that high concentrated protein solutions of more than 50 mg/ml usually exhibit also higher viscosities compared to lower concentrated protein solutions. Increased viscosity negatively affects the deliverability of the protein solutions especially in low application volumes and it may increase the injection time and pain at the site of injection. In addition to that, high viscosity impacts high-scale protein production in the industry. Thus, reducing viscosity of high concentrated protein solutions while maintaining stability for a long shelf life is i.a. important for the therapeutical in vivo setting.

Proteins in high concentrated solutions are often less stable than in diluted solutions, since the proteins tend to aggregate and may reversibly self-associate at higher concentrations. Aggregation may negatively impact structural integrity and therefore also the amount of functional, bioavailable protein in the therapeutical in vivo setting. This further complicates delivery by injection.

Solubility of proteins is another important quality criterion. Increased solubility of the isolated protein allows for the preparation of highly concentrated solutions required for the therapeutical in vivo setting.

Thus, providing a high concentrated protein solution with reduced viscosity and increased stability and solubility is beneficial for therapeutic applicability of therapeutic molecules.

To assess the CMC (Chemistry, Manufacturing, Control) parameters stability, solubility and viscosity of anti-Sema3A antibodies for potential therapeutic use, antibodies TPP-23289 and TPP-30788 (BI clone I) were diluted in PBS to 25 mg/ml and incubated at 700 rpm and 40° C. for two weeks. While antibodies are usually stored at 4°−10° C. for short-term or frozen at ≤−18° C. or ≥−81° C. for long term an exposure of mammalian antibodies to temperatures higher than ≥40° C. (mammalian average body temperature is 36° C.-39° C.) resembles a thermal stress condition. In this thermal stress condition accelerated protein stability/stress stability is tested. Analysis of stability was assessed by size-exclusion chromatography using a Superdex 200 column (Cytiva) coupled to an Äkta system (Cytiva) in PBS buffer as well as capillary gel electrophoresis using a Caliper system (Perkin Elmer). Changes in profile were calculated as percentage to non-stressed starting material. Solubility was determined by concentrating anti-Sema3A antibodies using an Amicon spin filter (Millipore) with a cut-off of 30 kDa in PBS buffer. The solubility was determined at 90% recovery from the concentrator and protein concentration was measured using Absorption at UV280 nm.

TABLE 24
Overview of CMC parameters for TPP-23298 and
TPP-30788; SEC = size-exclusion chromatography,
cGE = capillary gel electrophoresis

| CMC | | | | TPP-30788 |
|---|---|---|---|---|
| Parameter | Method | Analysis | TPP-23298 | (BI clone I) |
| Stability at | SEC* | Δ % monomer | 1 | −5.5 |
| 40° C. | cGE** | Δ % LC + HC | <1 | −4.7 |
| Solubility | concentrator | mg/ml at | 225 | 105 |
| | | 90% recovery | | |
| | SEC* | Δ % monomer | <1 | <1 |
| Viscosity | Viscosizer | cP | 5.1 | 5.3 |
| | | | (150 mg/ml) | (127 mg/ml) |

*SEC = Size exclusion chromatography;
**cGE = capillary gel electrophoresis

Stability, solubility and viscosity are critical CMC parameters for therapeutic molecules as described above. The structural integrity after a thermal stress condition, like exposure to 40° C., or concentrating step is analyzed via SEC and/or cGE to see the effect of the applied stress on the structural integrity. Less than 1% change after the applied stress compared to the start points to a stable molecule whereas deviations>1% points to instabilities in the molecule. TPP-23289 shows a much higher solubility in PBS compared to TPP-30788 by a factor>2 which is very beneficial for e.g. enabling low application volume. Furthermore, TPP-23298 is more stable and more resistant to heat stress than TPP-30788 and is less viscous in PBS buffer.

### Sequences

TABLE 1
Amino acid sequences and nucleic acid sequences of preferred
antibodies according to the present disclosure and of three
prior art antibodies. TPP-11489 corresponds to Chiome antibody
Humanized-2 derived of clone No. 4-2 strain (WO 2014/123186);
TPP-15051 respresents a murine IgG1 variant thereof. TPP-30788-
TPP-30791 corresponds to Böhringer Ingelheim antibody (BI)
Clone I-IV (WO 2020/225400). TPP-30792 corresponds to
University Ramot antibody clone I (WO 2020/261281).

| | | Sequence | Sequence | |
|---|---|---|---|---|
| TPP ID | Antibody Description | Region | Type | SEQ ID |
| TPP-11489 | Chiome Prior Art (hIgG1) | VH | PRT | SEQ ID NO: 1 |
| TPP-11489 | Chiome Prior Art (hIgG1) | HCDR1 | PRT | SEQ ID NO: 2 |
| TPP-11489 | Chiome Prior Art (hIgG1) | HCDR2 | PRT | SEQ ID NO: 3 |
| TPP-11489 | Chiome Prior Art (hIgG1) | HCDR3 | PRT | SEQ ID NO: 4 |
| TPP-11489 | Chiome Prior Art (hIgG1) | VL | PRT | SEQ ID NO: 5 |
| TPP-11489 | Chiome Prior Art (hIgG1) | LCDR1 | PRT | SEQ ID NO: 6 |
| TPP-11489 | Chiome Prior Art (hIgG1) | LCDR2 | PRT | SEQ ID NO: 7 |
| TPP-11489 | Chiome Prior Art (hIgG1) | LCDR3 | PRT | SEQ ID NO: 8 |
| TPP-11489 | Chiome Prior Art (hIgG1) | VH | DNA | SEQ ID NO: 9 |
| TPP-11489 | Chiome Prior Art (hIgG1) | HCDR1 | DNA | SEQ ID NO: 10 |
| TPP-11489 | Chiome Prior Art (hIgG1) | HCDR2 | DNA | SEQ ID NO: 11 |
| TPP-11489 | Chiome Prior Art (hIgG1) | HCDR3 | DNA | SEQ ID NO: 12 |
| TPP-11489 | Chiome Prior Art (hIgG1) | VL | DNA | SEQ ID NO: 13 |
| TPP-11489 | Chiome Prior Art (hIgG1) | LCDR1 | DNA | SEQ ID NO: 14 |
| TPP-11489 | Chiome Prior Art (hIgG1) | LCDR2 | DNA | SEQ ID NO: 15 |
| TPP-11489 | Chiome Prior Art (hIgG1) | LCDR3 | DNA | SEQ ID NO: 16 |
| TPP-11489 | Chiome Prior Art (hIgG1) | Heavy Chain | PRT | SEQ ID NO: 17 |
| TPP-11489 | Chiome Prior Art (hIgG1) | Light Chain | PRT | SEQ ID NO: 18 |
| TPP-11489 | Chiome Prior Art (hIgG1) | Heavy Chain | DNA | SEQ ID NO: 19 |
| TPP-11489 | Chiome Prior Art (hIgG1) | Light Chain | DNA | SEQ ID NO: 20 |
| TPP-15051 | Chiome Prior Art (mIgG1) | VH | PRT | SEQ ID NO: 21 |
| TPP-15051 | Chiome Prior Art (mIgG1) | HCDR1 | PRT | SEQ ID NO: 22 |
| TPP-15051 | Chiome Prior Art (mIgG1) | HCDR2 | PRT | SEQ ID NO: 23 |
| TPP-15051 | Chiome Prior Art (mIgG1) | HCDR3 | PRT | SEQ ID NO: 24 |
| TPP-15051 | Chiome Prior Art (mIgG1) | VL | PRT | SEQ ID NO: 25 |
| TPP-15051 | Chiome Prior Art (mIgG1) | LCDR1 | PRT | SEQ ID NO: 26 |
| TPP-15051 | Chiome Prior Art (mIgG1) | LCDR2 | PRT | SEQ ID NO: 27 |
| TPP-15051 | Chiome Prior Art (mIgG1) | LCDR3 | PRT | SEQ ID NO: 28 |
| TPP-15051 | Chiome Prior Art (mIgG1) | VH | DNA | SEQ ID NO: 29 |
| TPP-15051 | Chiome Prior Art (mIgG1) | HCDR1 | DNA | SEQ ID NO: 30 |
| TPP-15051 | Chiome Prior Art (mIgG1) | HCDR2 | DNA | SEQ ID NO: 31 |
| TPP-15051 | Chiome Prior Art (mIgG1) | HCDR3 | DNA | SEQ ID NO: 32 |
| TPP-15051 | Chiome Prior Art (mIgG1) | VL | DNA | SEQ ID NO: 33 |
| TPP-15051 | Chiome Prior Art (mIgG1) | LCDR1 | DNA | SEQ ID NO: 34 |
| TPP-15051 | Chiome Prior Art (mIgG1) | LCDR2 | DNA | SEQ ID NO: 35 |
| TPP-15051 | Chiome Prior Art (mIgG1) | LCDR3 | DNA | SEQ ID NO: 36 |
| TPP-15051 | Chiome Prior Art (mIgG1) | Heavy Chain | PRT | SEQ ID NO: 37 |
| TPP-15051 | Chiome Prior Art (mIgG1) | Light Chain | PRT | SEQ ID NO: 38 |
| TPP-15051 | Chiome Prior Art (mIgG1) | Heavy Chain | DNA | SEQ ID NO: 39 |
| TPP-15051 | Chiome Prior Art (mIgG1) | Light Chain | DNA | SEQ ID NO: 40 |
| TPP-15370 | IgG1, hit from panning | VH | PRT | SEQ ID NO: 41 |
| TPP-15370 | IgG1, hit from panning | HCDR1 | PRT | SEQ ID NO: 42 |
| TPP-15370 | IgG1, hit from panning | HCDR2 | PRT | SEQ ID NO: 43 |
| TPP-15370 | IgG1, hit from panning | HCDR3 | PRT | SEQ ID NO: 44 |
| TPP-15370 | IgG1, hit from panning | VL | PRT | SEQ ID NO: 45 |
| TPP-15370 | IgG1, hit from panning | LCDR1 | PRT | SEQ ID NO: 46 |
| TPP-15370 | IgG1, hit from panning | LCDR2 | PRT | SEQ ID NO: 47 |
| TPP-15370 | IgG1, hit from panning | LCDR3 | PRT | SEQ ID NO: 48 |
| TPP-15370 | IgG1, hit from panning | VH | DNA | SEQ ID NO: 49 |
| TPP-15370 | IgG1, hit from panning | HCDR1 | DNA | SEQ ID NO: 50 |
| TPP-15370 | IgG1, hit from panning | HCDR2 | DNA | SEQ ID NO: 51 |
| TPP-15370 | IgG1, hit from panning | HCDR3 | DNA | SEQ ID NO: 52 |
| TPP-15370 | IgG1, hit from panning | VL | DNA | SEQ ID NO: 53 |
| TPP-15370 | IgG1, hit from panning | LCDR1 | DNA | SEQ ID NO: 54 |
| TPP-15370 | IgG1, hit from panning | LCDR2 | DNA | SEQ ID NO: 55 |
| TPP-15370 | IgG1, hit from panning | LCDR3 | DNA | SEQ ID NO: 56 |
| TPP-15370 | IgG1, hit from panning | Heavy Chain | PRT | SEQ ID NO: 57 |
| TPP-15370 | IgG1, hit from panning | Light Chain | PRT | SEQ ID NO: 58 |
| TPP-15370 | IgG1, hit from panning | Heavy Chain | DNA | SEQ ID NO: 59 |
| TPP-15370 | IgG1, hit from panning | Light Chain | DNA | SEQ ID NO: 60 |
| TPP-15374 | IgG1, hit from panning | VH | PRT | SEQ ID NO: 61 |
| TPP-15374 | IgG1, hit from panning | HCDR1 | PRT | SEQ ID NO: 62 |
| TPP-15374 | IgG1, hit from panning | HCDR2 | PRT | SEQ ID NO: 63 |
| TPP-15374 | IgG1, hit from panning | HCDR3 | PRT | SEQ ID NO: 64 |
| TPP-15374 | IgG1, hit from panning | VL | PRT | SEQ ID NO: 65 |
| TPP-15374 | IgG1, hit from panning | LCDR1 | PRT | SEQ ID NO: 66 |
| TPP-15374 | IgG1, hit from panning | LCDR2 | PRT | SEQ ID NO: 67 |
| TPP-15374 | IgG1, hit from panning | LCDR3 | PRT | SEQ ID NO: 68 |
| TPP-15374 | IgG1, hit from panning | VH | DNA | SEQ ID NO: 69 |
| TPP-15374 | IgG1, hit from panning | HCDR1 | DNA | SEQ ID NO: 70 |
| TPP-15374 | IgG1, hit from panning | HCDR2 | DNA | SEQ ID NO: 71 |
| TPP-15374 | IgG1, hit from panning | HCDR3 | DNA | SEQ ID NO: 72 |
| TPP-15374 | IgG1, hit from panning | VL | DNA | SEQ ID NO: 73 |
| TPP-15374 | IgG1, hit from panning | LCDR1 | DNA | SEQ ID NO: 74 |
| TPP-15374 | IgG1, hit from panning | LCDR2 | DNA | SEQ ID NO: 75 |
| TPP-15374 | IgG1, hit from panning | LCDR3 | DNA | SEQ ID NO: 76 |
| TPP-15374 | IgG1, hit from panning | Heavy Chain | PRT | SEQ ID NO: 77 |
| TPP-15374 | IgG1, hit from panning | Light Chain | PRT | SEQ ID NO: 78 |
| TPP-15374 | IgG1, hit from panning | Heavy Chain | DNA | SEQ ID NO: 79 |
| TPP-15374 | IgG1, hit from panning | Light Chain | DNA | SEQ ID NO: 80 |
| TPP-17755 | Samsung Prior Art F11 | VH | PRT | SEQ ID NO: 81 |
| TPP-17755 | Samsung Prior Art F11 | HCDR1 | PRT | SEQ ID NO: 82 |
| TPP-17755 | Samsung Prior Art F11 | HCDR2 | PRT | SEQ ID NO: 83 |
| TPP-17755 | Samsung Prior Art F11 | HCDR3 | PRT | SEQ ID NO: 84 |
| TPP-17755 | Samsung Prior Art F11 | VL | PRT | SEQ ID NO: 85 |
| TPP-17755 | Samsung Prior Art F11 | LCDR1 | PRT | SEQ ID NO: 86 |
| TPP-17755 | Samsung Prior Art F11 | LCDR2 | PRT | SEQ ID NO: 87 |
| TPP-17755 | Samsung Prior Art F11 | LCDR3 | PRT | SEQ ID NO: 88 |
| TPP-17755 | Samsung Prior Art F11 | VH | DNA | SEQ ID NO: 89 |
| TPP-17755 | Samsung Prior Art F11 | HCDR1 | DNA | SEQ ID NO: 90 |
| TPP-17755 | Samsung Prior Art F11 | HCDR2 | DNA | SEQ ID NO: 91 |
| TPP-17755 | Samsung Prior Art F11 | HCDR3 | DNA | SEQ ID NO: 92 |
| TPP-17755 | Samsung Prior Art F11 | VL | DNA | SEQ ID NO: 93 |
| TPP-17755 | Samsung Prior Art F11 | LCDR1 | DNA | SEQ ID NO: 94 |
| TPP-17755 | Samsung Prior Art F11 | LCDR2 | DNA | SEQ ID NO: 95 |
| TPP-17755 | Samsung Prior Art F11 | LCDR3 | DNA | SEQ ID NO: 96 |
| TPP-17755 | Samsung Prior Art F11 | Heavy Chain | PRT | SEQ ID NO: 97 |
| TPP-17755 | Samsung Prior Art F11 | Light Chain | PRT | SEQ ID NO: 98 |
| TPP-17755 | Samsung Prior Art F11 | Heavy Chain | DNA | SEQ ID NO: 99 |
| TPP-17755 | Samsung Prior Art F11 | Light Chain | DNA | SEQ ID NO: 100 |
| TPP-18533 | germline IgG1 of TPP-15374 | VH | PRT | SEQ ID NO: 101 |
| TPP-18533 | germline IgG1 of TPP-15374 | HCDR1 | PRT | SEQ ID NO: 102 |
| TPP-18533 | germline IgG1 of TPP-15374 | HCDR2 | PRT | SEQ ID NO: 103 |
| TPP-18533 | germline IgG1 of TPP-15374 | HCDR3 | PRT | SEQ ID NO: 104 |
| TPP-18533 | germline IgG1 of TPP-15374 | VL | PRT | SEQ ID NO: 105 |
| TPP-18533 | germline IgG1 of TPP-15374 | LCDR1 | PRT | SEQ ID NO: 106 |
| TPP-18533 | germline IgG1 of TPP-15374 | LCDR2 | PRT | SEQ ID NO: 107 |
| TPP-18533 | germline IgG1 of TPP-15374 | LCDR3 | PRT | SEQ ID NO: 108 |
| TPP-18533 | germline IgG1 of TPP-15374 | VH | DNA | SEQ ID NO: 109 |
| TPP-18533 | germline IgG1 of TPP-15374 | HCDR1 | DNA | SEQ ID NO: 110 |
| TPP-18533 | germline IgG1 of TPP-15374 | HCDR2 | DNA | SEQ ID NO: 111 |
| TPP-18533 | germline IgG1 of TPP-15374 | HCDR3 | DNA | SEQ ID NO: 112 |
| TPP-18533 | germline IgG1 of TPP-15374 | VL | DNA | SEQ ID NO: 113 |
| TPP-18533 | germline IgG1 of TPP-15374 | LCDR1 | DNA | SEQ ID NO: 114 |
| TPP-18533 | germline IgG1 of TPP-15374 | LCDR2 | DNA | SEQ ID NO: 115 |
| TPP-18533 | germline IgG1 of TPP-15374 | LCDR3 | DNA | SEQ ID NO: 116 |
| TPP-18533 | germline IgG1 of TPP-15374 | Heavy Chain | PRT | SEQ ID NO: 117 |
| TPP-18533 | germline IgG1 of TPP-15374 | Light Chain | PRT | SEQ ID NO: 118 |
| TPP-18533 | germline IgG1 of TPP-15374 | Heavy Chain | DNA | SEQ ID NO: 119 |
| TPP-18533 | germline IgG1 of TPP-15374 | Light Chain | DNA | SEQ ID NO: 120 |
| TPP-21565 | germline IgG1 of TPP-15370 | VH | PRT | SEQ ID NO: 121 |
| TPP-21565 | germline IgG1 of TPP-15370 | HCDR1 | PRT | SEQ ID NO: 122 |
| TPP-21565 | germline IgG1 of TPP-15370 | HCDR2 | PRT | SEQ ID NO: 123 |
| TPP-21565 | germline IgG1 of TPP-15370 | HCDR3 | PRT | SEQ ID NO: 124 |
| TPP-21565 | germline IgG1 of TPP-15370 | VL | PRT | SEQ ID NO: 125 |
| TPP-21565 | germline IgG1 of TPP-15370 | LCDR1 | PRT | SEQ ID NO: 126 |
| TPP-21565 | germline IgG1 of TPP-15370 | LCDR2 | PRT | SEQ ID NO: 127 |
| TPP-21565 | germline IgG1 of TPP-15370 | LCDR3 | PRT | SEQ ID NO: 128 |
| TPP-21565 | germline IgG1 of TPP-15370 | VH | DNA | SEQ ID NO: 129 |
| TPP-21565 | germline IgG1 of TPP-15370 | HCDR1 | DNA | SEQ ID NO: 130 |
| TPP-21565 | germline IgG1 of TPP-15370 | HCDR2 | DNA | SEQ ID NO: 131 |
| TPP-21565 | germline IgG1 of TPP-15370 | HCDR3 | DNA | SEQ ID NO: 132 |
| TPP-21565 | germline IgG1 of TPP-15370 | VL | DNA | SEQ ID NO: 133 |
| TPP-21565 | germline IgG1 of TPP-15370 | LCDR1 | DNA | SEQ ID NO: 134 |
| TPP-21565 | germline IgG1 of TPP-15370 | LCDR2 | DNA | SEQ ID NO: 135 |
| TPP-21565 | germline IgG1 of TPP-15370 | LCDR3 | DNA | SEQ ID NO: 136 |
| TPP-21565 | germline IgG1 of TPP-15370 | Heavy Chain | PRT | SEQ ID NO: 137 |
| TPP-21565 | germline IgG1 of TPP-15370 | Light Chain | PRT | SEQ ID NO: 138 |
| TPP-21565 | germline IgG1 of TPP-15370 | Heavy Chain | DNA | SEQ ID NO: 139 |
| TPP-21565 | germline IgG1 of TPP-15370 | Light Chain | DNA | SEQ ID NO: 140 |
| TPP-23298 | Recombi Variant of TPP-15370 | VH | PRT | SEQ ID NO: 141 |
| TPP-23298 | Recombi Variant of TPP-15370 | HCDR1 | PRT | SEQ ID NO: 142 |
| TPP-23298 | Recombi Variant of TPP-15370 | HCDR2 | PRT | SEQ ID NO: 143 |
| TPP-23298 | Recombi Variant of TPP-15370 | HCDR3 | PRT | SEQ ID NO: 144 |
| TPP-23298 | Recombi Variant of TPP-15370 | VL | PRT | SEQ ID NO: 145 |
| TPP-23298 | Recombi Variant of TPP-15370 | LCDR1 | PRT | SEQ ID NO: 146 |
| TPP-23298 | Recombi Variant of TPP-15370 | LCDR2 | PRT | SEQ ID NO: 147 |
| TPP-23298 | Recombi Variant of TPP-15370 | LCDR3 | PRT | SEQ ID NO: 148 |
| TPP-23298 | Recombi Variant of TPP-15370 | VH | DNA | SEQ ID NO: 149 |
| TPP-23298 | Recombi Variant of TPP-15370 | HCDR1 | DNA | SEQ ID NO: 150 |
| TPP-23298 | Recombi Variant of TPP-15370 | HCDR2 | DNA | SEQ ID NO: 151 |
| TPP-23298 | Recombi Variant of TPP-15370 | HCDR3 | DNA | SEQ ID NO: 152 |
| TPP-23298 | Recombi Variant of TPP-15370 | VL | DNA | SEQ ID NO: 153 |
| TPP-23298 | Recombi Variant of TPP-15370 | LCDR1 | DNA | SEQ ID NO: 154 |
| TPP-23298 | Recombi Variant of TPP-15370 | LCDR2 | DNA | SEQ ID NO: 155 |
| TPP-23298 | Recombi Variant of TPP-15370 | LCDR3 | DNA | SEQ ID NO: 156 |
| TPP-23298 | Recombi Variant of TPP-15370 | Heavy Chain | PRT | SEQ ID NO: 157 |
| TPP-23298 | Recombi Variant of TPP-15370 | Light Chain | PRT | SEQ ID NO: 158 |
| TPP-23298 | Recombi Variant of TPP-15370 | Heavy Chain | DNA | SEQ ID NO: 159 |
| TPP-23298 | Recombi Variant of TPP-15370 | Light Chain | DNA | SEQ ID NO: 160 |
| TPP-23334 | Recombi Variant of TPP-15370 | VH | PRT | SEQ ID NO: 161 |
| TPP-23334 | Recombi Variant of TPP-15370 | HCDR1 | PRT | SEQ ID NO: 162 |
| TPP-23334 | Recombi Variant of TPP-15370 | HCDR2 | PRT | SEQ ID NO: 163 |
| TPP-23334 | Recombi Variant of TPP-15370 | HCDR3 | PRT | SEQ ID NO: 164 |
| TPP-23334 | Recombi Variant of TPP-15370 | VL | PRT | SEQ ID NO: 165 |
| TPP-23334 | Recombi Variant of TPP-15370 | LCDR1 | PRT | SEQ ID NO: 166 |
| TPP-23334 | Recombi Variant of TPP-15370 | LCDR2 | PRT | SEQ ID NO: 167 |
| TPP-23334 | Recombi Variant of TPP-15370 | LCDR3 | PRT | SEQ ID NO: 168 |
| TPP-23334 | Recombi Variant of TPP-15370 | VH | DNA | SEQ ID NO: 169 |
| TPP-23334 | Recombi Variant of TPP-15370 | HCDR1 | DNA | SEQ ID NO: 170 |
| TPP-23334 | Recombi Variant of TPP-15370 | HCDR2 | DNA | SEQ ID NO: 171 |
| TPP-23334 | Recombi Variant of TPP-15370 | HCDR3 | DNA | SEQ ID NO: 172 |
| TPP-23334 | Recombi Variant of TPP-15370 | VL | DNA | SEQ ID NO: 173 |
| TPP-23334 | Recombi Variant of TPP-15370 | LCDR1 | DNA | SEQ ID NO: 174 |
| TPP-23334 | Recombi Variant of TPP-15370 | LCDR2 | DNA | SEQ ID NO: 175 |
| TPP-23334 | Recombi Variant of TPP-15370 | LCDR3 | DNA | SEQ ID NO: 176 |
| TPP-23334 | Recombi Variant of TPP-15370 | Heavy Chain | PRT | SEQ ID NO: 177 |
| TPP-23334 | Recombi Variant of TPP-15370 | Light Chain | PRT | SEQ ID NO: 178 |
| TPP-23334 | Recombi Variant of TPP-15370 | Heavy Chain | DNA | SEQ ID NO: 179 |
| TPP-23334 | Recombi Variant of TPP-15370 | Light Chain | DNA | SEQ ID NO: 180 |
| TPP-23337 | Recombi Variant of TPP-15370 | VH | PRT | SEQ ID NO: 181 |
| TPP-23337 | Recombi Variant of TPP-15370 | HCDR1 | PRT | SEQ ID NO: 182 |
| TPP-23337 | Recombi Variant of TPP-15370 | HCDR2 | PRT | SEQ ID NO: 183 |
| TPP-23337 | Recombi Variant of TPP-15370 | HCDR3 | PRT | SEQ ID NO: 184 |
| TPP-23337 | Recombi Variant of TPP-15370 | VL | PRT | SEQ ID NO: 185 |
| TPP-23337 | Recombi Variant of TPP-15370 | LCDR1 | PRT | SEQ ID NO: 186 |
| TPP-23337 | Recombi Variant of TPP-15370 | LCDR2 | PRT | SEQ ID NO: 187 |
| TPP-23337 | Recombi Variant of TPP-15370 | LCDR3 | PRT | SEQ ID NO: 188 |
| TPP-23337 | Recombi Variant of TPP-15370 | VH | DNA | SEQ ID NO: 189 |
| TPP-23337 | Recombi Variant of TPP-15370 | HCDR1 | DNA | SEQ ID NO: 190 |
| TPP-23337 | Recombi Variant of TPP-15370 | HCDR2 | DNA | SEQ ID NO: 191 |
| TPP-23337 | Recombi Variant of TPP-15370 | HCDR3 | DNA | SEQ ID NO: 192 |
| TPP-23337 | Recombi Variant of TPP-15370 | VL | DNA | SEQ ID NO: 193 |
| TPP-23337 | Recombi Variant of TPP-15370 | LCDR1 | DNA | SEQ ID NO: 194 |
| TPP-23337 | Recombi Variant of TPP-15370 | LCDR2 | DNA | SEQ ID NO: 195 |
| TPP-23337 | Recombi Variant of TPP-15370 | LCDR3 | DNA | SEQ ID NO: 196 |
| TPP-23337 | Recombi Variant of TPP-15370 | Heavy Chain | PRT | SEQ ID NO: 197 |
| TPP-23337 | Recombi Variant of TPP-15370 | Light Chain | PRT | SEQ ID NO: 198 |
| TPP-23337 | Recombi Variant of TPP-15370 | Heavy Chain | DNA | SEQ ID NO: 199 |
| TPP-23337 | Recombi Variant of TPP-15370 | Light Chain | DNA | SEQ ID NO: 200 |
| TPP-23338 | Recombi Variant of TPP-15370 | VH | PRT | SEQ ID NO: 201 |
| TPP-23338 | Recombi Variant of TPP-15370 | HCDR1 | PRT | SEQ ID NO: 202 |
| TPP-23338 | Recombi Variant of TPP-15370 | HCDR2 | PRT | SEQ ID NO: 203 |
| TPP-23338 | Recombi Variant of TPP-15370 | HCDR3 | PRT | SEQ ID NO: 204 |
| TPP-23338 | Recombi Variant of TPP-15370 | VL | PRT | SEQ ID NO: 205 |
| TPP-23338 | Recombi Variant of TPP-15370 | LCDR1 | PRT | SEQ ID NO: 206 |
| TPP-23338 | Recombi Variant of TPP-15370 | LCDR2 | PRT | SEQ ID NO: 207 |
| TPP-23338 | Recombi Variant of TPP-15370 | LCDR3 | PRT | SEQ ID NO: 208 |
| TPP-23338 | Recombi Variant of TPP-15370 | VH | DNA | SEQ ID NO: 209 |
| TPP-23338 | Recombi Variant of TPP-15370 | HCDR1 | DNA | SEQ ID NO: 210 |
| TPP-23338 | Recombi Variant of TPP-15370 | HCDR2 | DNA | SEQ ID NO: 211 |
| TPP-23338 | Recombi Variant of TPP-15370 | HCDR3 | DNA | SEQ ID NO: 212 |
| TPP-23338 | Recombi Variant of TPP-15370 | VL | DNA | SEQ ID NO: 213 |
| TPP-23338 | Recombi Variant of TPP-15370 | LCDR1 | DNA | SEQ ID NO: 214 |
| TPP-23338 | Recombi Variant of TPP-15370 | LCDR2 | DNA | SEQ ID NO: 215 |
| TPP-23338 | Recombi Variant of TPP-15370 | LCDR3 | DNA | SEQ ID NO: 216 |
| TPP-23338 | Recombi Variant of TPP-15370 | Heavy Chain | PRT | SEQ ID NO: 217 |
| TPP-23338 | Recombi Variant of TPP-15370 | Light Chain | PRT | SEQ ID NO: 218 |
| TPP-23338 | Recombi Variant of TPP-15370 | Heavy Chain | DNA | SEQ ID NO: 219 |
| TPP-23338 | Recombi Variant of TPP-15370 | Light Chain | DNA | SEQ ID NO: 220 |
| TPP-23340 | Recombi Variant of TPP-15370 | VH | PRT | SEQ ID NO: 221 |
| TPP-23340 | Recombi Variant of TPP-15370 | HCDR1 | PRT | SEQ ID NO: 222 |
| TPP-23340 | Recombi Variant of TPP-15370 | HCDR2 | PRT | SEQ ID NO: 223 |
| TPP-23340 | Recombi Variant of TPP-15370 | HCDR3 | PRT | SEQ ID NO: 224 |
| TPP-23340 | Recombi Variant of TPP-15370 | VL | PRT | SEQ ID NO: 225 |
| TPP-23340 | Recombi Variant of TPP-15370 | LCDR1 | PRT | SEQ ID NO: 226 |
| TPP-23340 | Recombi Variant of TPP-15370 | LCDR2 | PRT | SEQ ID NO: 227 |
| TPP-23340 | Recombi Variant of TPP-15370 | LCDR3 | PRT | SEQ ID NO: 228 |
| TPP-23340 | Recombi Variant of TPP-15370 | VH | DNA | SEQ ID NO: 229 |
| TPP-23340 | Recombi Variant of TPP-15370 | HCDR1 | DNA | SEQ ID NO: 230 |
| TPP-23340 | Recombi Variant of TPP-15370 | HCDR2 | DNA | SEQ ID NO: 231 |
| TPP-23340 | Recombi Variant of TPP-15370 | HCDR3 | DNA | SEQ ID NO: 232 |
| TPP-23340 | Recombi Variant of TPP-15370 | VL | DNA | SEQ ID NO: 233 |
| TPP-23340 | Recombi Variant of TPP-15370 | LCDR1 | DNA | SEQ ID NO: 234 |
| TPP-23340 | Recombi Variant of TPP-15370 | LCDR2 | DNA | SEQ ID NO: 235 |
| TPP-23340 | Recombi Variant of TPP-15370 | LCDR3 | DNA | SEQ ID NO: 236 |
| TPP-23340 | Recombi Variant of TPP-15370 | Heavy Chain | PRT | SEQ ID NO: 237 |
| TPP-23340 | Recombi Variant of TPP-15370 | Light Chain | PRT | SEQ ID NO: 238 |
| TPP-23340 | Recombi Variant of TPP-15370 | Heavy Chain | DNA | SEQ ID NO: 239 |
| TPP-23340 | Recombi Variant of TPP-15370 | Light Chain | DNA | SEQ ID NO: 240 |
| TPP-23341 | Recombi Variant of TPP-15370 | VH | PRT | SEQ ID NO: 241 |
| TPP-23341 | Recombi Variant of TPP-15370 | HCDR1 | PRT | SEQ ID NO: 242 |
| TPP-23341 | Recombi Variant of TPP-15370 | HCDR2 | PRT | SEQ ID NO: 243 |
| TPP-23341 | Recombi Variant of TPP-15370 | HCDR3 | PRT | SEQ ID NO: 244 |
| TPP-23341 | Recombi Variant of TPP-15370 | VL | PRT | SEQ ID NO: 245 |
| TPP-23341 | Recombi Variant of TPP-15370 | LCDR1 | PRT | SEQ ID NO: 246 |
| TPP-23341 | Recombi Variant of TPP-15370 | LCDR2 | PRT | SEQ ID NO: 247 |
| TPP-23341 | Recombi Variant of TPP-15370 | LCDR3 | PRT | SEQ ID NO: 248 |
| TPP-23341 | Recombi Variant of TPP-15370 | VH | DNA | SEQ ID NO: 249 |
| TPP-23341 | Recombi Variant of TPP-15370 | HCDR1 | DNA | SEQ ID NO: 250 |
| TPP-23341 | Recombi Variant of TPP-15370 | HCDR2 | DNA | SEQ ID NO: 251 |
| TPP-23341 | Recombi Variant of TPP-15370 | HCDR3 | DNA | SEQ ID NO: 252 |
| TPP-23341 | Recombi Variant of TPP-15370 | VL | DNA | SEQ ID NO: 253 |
| TPP-23341 | Recombi Variant of TPP-15370 | LCDR1 | DNA | SEQ ID NO: 254 |
| TPP-23341 | Recombi Variant of TPP-15370 | LCDR2 | DNA | SEQ ID NO: 255 |
| TPP-23341 | Recombi Variant of TPP-15370 | LCDR3 | DNA | SEQ ID NO: 256 |
| TPP-23341 | Recombi Variant of TPP-15370 | Heavy Chain | PRT | SEQ ID NO: 257 |
| TPP-23341 | Recombi Variant of TPP-15370 | Light Chain | PRT | SEQ ID NO: 258 |
| TPP-23341 | Recombi Variant of TPP-15370 | Heavy Chain | DNA | SEQ ID NO: 259 |
| TPP-23341 | Recombi Variant of TPP-15370 | Light Chain | DNA | SEQ ID NO: 260 |
| TPP-23345 | Recombi Variant of TPP-15370 | VH | PRT | SEQ ID NO: 261 |
| TPP-23345 | Recombi Variant of TPP-15370 | HCDR1 | PRT | SEQ ID NO: 262 |
| TPP-23345 | Recombi Variant of TPP-15370 | HCDR2 | PRT | SEQ ID NO: 263 |
| TPP-23345 | Recombi Variant of TPP-15370 | HCDR3 | PRT | SEQ ID NO: 264 |
| TPP-23345 | Recombi Variant of TPP-15370 | VL | PRT | SEQ ID NO: 265 |
| TPP-23345 | Recombi Variant of TPP-15370 | LCDR1 | PRT | SEQ ID NO: 266 |
| TPP-23345 | Recombi Variant of TPP-15370 | LCDR2 | PRT | SEQ ID NO: 267 |
| TPP-23345 | Recombi Variant of TPP-15370 | LCDR3 | PRT | SEQ ID NO: 268 |
| TPP-23345 | Recombi Variant of TPP-15370 | VH | DNA | SEQ ID NO: 269 |
| TPP-23345 | Recombi Variant of TPP-15370 | HCDR1 | DNA | SEQ ID NO: 270 |
| TPP-23345 | Recombi Variant of TPP-15370 | HCDR2 | DNA | SEQ ID NO: 271 |
| TPP-23345 | Recombi Variant of TPP-15370 | HCDR3 | DNA | SEQ ID NO: 272 |
| TPP-23345 | Recombi Variant of TPP-15370 | VL | DNA | SEQ ID NO: 273 |
| TPP-23345 | Recombi Variant of TPP-15370 | LCDR1 | DNA | SEQ ID NO: 274 |
| TPP-23345 | Recombi Variant of TPP-15370 | LCDR2 | DNA | SEQ ID NO: 275 |
| TPP-23345 | Recombi Variant of TPP-15370 | LCDR3 | DNA | SEQ ID NO: 276 |
| TPP-23345 | Recombi Variant of TPP-15370 | Heavy Chain | PRT | SEQ ID NO: 277 |
| TPP-23345 | Recombi Variant of TPP-15370 | Light Chain | PRT | SEQ ID NO: 278 |
| TPP-23345 | Recombi Variant of TPP-15370 | Heavy Chain | DNA | SEQ ID NO: 279 |
| TPP-23345 | Recombi Variant of TPP-15370 | Light Chain | DNA | SEQ ID NO: 280 |
| TPP-23346 | Recombi Variant of TPP-15370 | VH | PRT | SEQ ID NO: 281 |
| TPP-23346 | Recombi Variant of TPP-15370 | HCDR1 | PRT | SEQ ID NO: 282 |
| TPP-23346 | Recombi Variant of TPP-15370 | HCDR2 | PRT | SEQ ID NO: 283 |
| TPP-23346 | Recombi Variant of TPP-15370 | HCDR3 | PRT | SEQ ID NO: 284 |
| TPP-23346 | Recombi Variant of TPP-15370 | VL | PRT | SEQ ID NO: 285 |
| TPP-23346 | Recombi Variant of TPP-15370 | LCDR1 | PRT | SEQ ID NO: 286 |
| TPP-23346 | Recombi Variant of TPP-15370 | LCDR2 | PRT | SEQ ID NO: 287 |
| TPP-23346 | Recombi Variant of TPP-15370 | LCDR3 | PRT | SEQ ID NO: 288 |
| TPP-23346 | Recombi Variant of TPP-15370 | VH | DNA | SEQ ID NO: 289 |
| TPP-23346 | Recombi Variant of TPP-15370 | HCDR1 | DNA | SEQ ID NO: 290 |
| TPP-23346 | Recombi Variant of TPP-15370 | HCDR2 | DNA | SEQ ID NO: 291 |
| TPP-23346 | Recombi Variant of TPP-15370 | HCDR3 | DNA | SEQ ID NO: 292 |
| TPP-23346 | Recombi Variant of TPP-15370 | VL | DNA | SEQ ID NO: 293 |
| TPP-23346 | Recombi Variant of TPP-15370 | LCDR1 | DNA | SEQ ID NO: 294 |
| TPP-23346 | Recombi Variant of TPP-15370 | LCDR2 | DNA | SEQ ID NO: 295 |
| TPP-23346 | Recombi Variant of TPP-15370 | LCDR3 | DNA | SEQ ID NO: 296 |
| TPP-23346 | Recombi Variant of TPP-15370 | Heavy Chain | PRT | SEQ ID NO: 297 |
| TPP-23346 | Recombi Variant of TPP-15370 | Light Chain | PRT | SEQ ID NO: 298 |
| TPP-23346 | Recombi Variant of TPP-15370 | Heavy Chain | DNA | SEQ ID NO: 299 |
| TPP-23346 | Recombi Variant of TPP-15370 | Light Chain | DNA | SEQ ID NO: 300 |
| TPP-23347 | Recombi Variant of TPP-15370 | VH | PRT | SEQ ID NO: 301 |
| TPP-23347 | Recombi Variant of TPP-15370 | HCDR1 | PRT | SEQ ID NO: 302 |
| TPP-23347 | Recombi Variant of TPP-15370 | HCDR2 | PRT | SEQ ID NO: 303 |
| TPP-23347 | Recombi Variant of TPP-15370 | HCDR3 | PRT | SEQ ID NO: 304 |
| TPP-23347 | Recombi Variant of TPP-15370 | VL | PRT | SEQ ID NO: 305 |
| TPP-23347 | Recombi Variant of TPP-15370 | LCDR1 | PRT | SEQ ID NO: 306 |
| TPP-23347 | Recombi Variant of TPP-15370 | LCDR2 | PRT | SEQ ID NO: 307 |
| TPP-23347 | Recombi Variant of TPP-15370 | LCDR3 | PRT | SEQ ID NO: 308 |
| TPP-23347 | Recombi Variant of TPP-15370 | VH | DNA | SEQ ID NO: 309 |
| TPP-23347 | Recombi Variant of TPP-15370 | HCDR1 | DNA | SEQ ID NO: 310 |
| TPP-23347 | Recombi Variant of TPP-15370 | HCDR2 | DNA | SEQ ID NO: 311 |
| TPP-23347 | Recombi Variant of TPP-15370 | HCDR3 | DNA | SEQ ID NO: 312 |
| TPP-23347 | Recombi Variant of TPP-15370 | VL | DNA | SEQ ID NO: 313 |
| TPP-23347 | Recombi Variant of TPP-15370 | LCDR1 | DNA | SEQ ID NO: 314 |
| TPP-23347 | Recombi Variant of TPP-15370 | LCDR2 | DNA | SEQ ID NO: 315 |
| TPP-23347 | Recombi Variant of TPP-15370 | LCDR3 | DNA | SEQ ID NO: 316 |
| TPP-23347 | Recombi Variant of TPP-15370 | Heavy Chain | PRT | SEQ ID NO: 317 |
| TPP-23347 | Recombi Variant of TPP-15370 | Light Chain | PRT | SEQ ID NO: 318 |
| TPP-23347 | Recombi Variant of TPP-15370 | Heavy Chain | DNA | SEQ ID NO: 319 |
| TPP-23347 | Recombi Variant of TPP-15370 | Light Chain | DNA | SEQ ID NO: 320 |
| TPP-23373 | Recombi Variant of TPP-15370 | VH | PRT | SEQ ID NO: 321 |
| TPP-23373 | Recombi Variant of TPP-15370 | HCDR1 | PRT | SEQ ID NO: 322 |
| TPP-23373 | Recombi Variant of TPP-15370 | HCDR2 | PRT | SEQ ID NO: 323 |
| TPP-23373 | Recombi Variant of TPP-15370 | HCDR3 | PRT | SEQ ID NO: 324 |
| TPP-23373 | Recombi Variant of TPP-15370 | VL | PRT | SEQ ID NO: 325 |
| TPP-23373 | Recombi Variant of TPP-15370 | LCDR1 | PRT | SEQ ID NO: 326 |
| TPP-23373 | Recombi Variant of TPP-15370 | LCDR2 | PRT | SEQ ID NO: 327 |
| TPP-23373 | Recombi Variant of TPP-15370 | LCDR3 | PRT | SEQ ID NO: 328 |
| TPP-23373 | Recombi Variant of TPP-15370 | VH | DNA | SEQ ID NO: 329 |
| TPP-23373 | Recombi Variant of TPP-15370 | HCDR1 | DNA | SEQ ID NO: 330 |
| TPP-23373 | Recombi Variant of TPP-15370 | HCDR2 | DNA | SEQ ID NO: 331 |
| TPP-23373 | Recombi Variant of TPP-15370 | HCDR3 | DNA | SEQ ID NO: 332 |
| TPP-23373 | Recombi Variant of TPP-15370 | VL | DNA | SEQ ID NO: 333 |
| TPP-23373 | Recombi Variant of TPP-15370 | LCDR1 | DNA | SEQ ID NO: 334 |
| TPP-23373 | Recombi Variant of TPP-15370 | LCDR2 | DNA | SEQ ID NO: 335 |
| TPP-23373 | Recombi Variant of TPP-15370 | LCDR3 | DNA | SEQ ID NO: 336 |
| TPP-23373 | Recombi Variant of TPP-15370 | Heavy Chain | PRT | SEQ ID NO: 337 |
| TPP-23373 | Recombi Variant of TPP-15370 | Light Chain | PRT | SEQ ID NO: 338 |
| TPP-23373 | Recombi Variant of TPP-15370 | Heavy Chain | DNA | SEQ ID NO: 339 |
| TPP-23373 | Recombi Variant of TPP-15370 | Light Chain | DNA | SEQ ID NO: 340 |
| TPP-23374 | Recombi Variant of TPP-15370 | VH | PRT | SEQ ID NO: 341 |
| TPP-23374 | Recombi Variant of TPP-15370 | HCDR1 | PRT | SEQ ID NO: 342 |
| TPP-23374 | Recombi Variant of TPP-15370 | HCDR2 | PRT | SEQ ID NO: 343 |
| TPP-23374 | Recombi Variant of TPP-15370 | HCDR3 | PRT | SEQ ID NO: 344 |
| TPP-23374 | Recombi Variant of TPP-15370 | VL | PRT | SEQ ID NO: 345 |
| TPP-23374 | Recombi Variant of TPP-15370 | LCDR1 | PRT | SEQ ID NO: 346 |
| TPP-23374 | Recombi Variant of TPP-15370 | LCDR2 | PRT | SEQ ID NO: 347 |
| TPP-23374 | Recombi Variant of TPP-15370 | LCDR3 | PRT | SEQ ID NO: 348 |
| TPP-23374 | Recombi Variant of TPP-15370 | VH | DNA | SEQ ID NO: 349 |
| TPP-23374 | Recombi Variant of TPP-15370 | HCDR1 | DNA | SEQ ID NO: 350 |
| TPP-23374 | Recombi Variant of TPP-15370 | HCDR2 | DNA | SEQ ID NO: 351 |
| TPP-23374 | Recombi Variant of TPP-15370 | HCDR3 | DNA | SEQ ID NO: 352 |
| TPP-23374 | Recombi Variant of TPP-15370 | VL | DNA | SEQ ID NO: 353 |
| TPP-23374 | Recombi Variant of TPP-15370 | LCDR1 | DNA | SEQ ID NO: 354 |
| TPP-23374 | Recombi Variant of TPP-15370 | LCDR2 | DNA | SEQ ID NO: 355 |
| TPP-23374 | Recombi Variant of TPP-15370 | LCDR3 | DNA | SEQ ID NO: 356 |
| TPP-23374 | Recombi Variant of TPP-15370 | Heavy Chain | PRT | SEQ ID NO: 357 |
| TPP-23374 | Recombi Variant of TPP-15370 | Light Chain | PRT | SEQ ID NO: 358 |
| TPP-23374 | Recombi Variant of TPP-15370 | Heavy Chain | DNA | SEQ ID NO: 359 |
| TPP-23374 | Recombi Variant of TPP-15370 | Light Chain | DNA | SEQ ID NO: 360 |
| TPP-23375 | Recombi Variant of TPP-15370 | VH | PRT | SEQ ID NO: 361 |
| TPP-23375 | Recombi Variant of TPP-15370 | HCDR1 | PRT | SEQ ID NO: 362 |
| TPP-23375 | Recombi Variant of TPP-15370 | HCDR2 | PRT | SEQ ID NO: 363 |
| TPP-23375 | Recombi Variant of TPP-15370 | HCDR3 | PRT | SEQ ID NO: 364 |
| TPP-23375 | Recombi Variant of TPP-15370 | VL | PRT | SEQ ID NO: 365 |
| TPP-23375 | Recombi Variant of TPP-15370 | LCDR1 | PRT | SEQ ID NO: 366 |
| TPP-23375 | Recombi Variant of TPP-15370 | LCDR2 | PRT | SEQ ID NO: 367 |
| TPP-23375 | Recombi Variant of TPP-15370 | LCDR3 | PRT | SEQ ID NO: 368 |
| TPP-23375 | Recombi Variant of TPP-15370 | VH | DNA | SEQ ID NO: 369 |
| TPP-23375 | Recombi Variant of TPP-15370 | HCDR1 | DNA | SEQ ID NO: 370 |
| TPP-23375 | Recombi Variant of TPP-15370 | HCDR2 | DNA | SEQ ID NO: 371 |
| TPP-23375 | Recombi Variant of TPP-15370 | HCDR3 | DNA | SEQ ID NO: 372 |
| TPP-23375 | Recombi Variant of TPP-15370 | VL | DNA | SEQ ID NO: 373 |
| TPP-23375 | Recombi Variant of TPP-15370 | LCDR1 | DNA | SEQ ID NO: 374 |
| TPP-23375 | Recombi Variant of TPP-15370 | LCDR2 | DNA | SEQ ID NO: 375 |
| TPP-23375 | Recombi Variant of TPP-15370 | LCDR3 | DNA | SEQ ID NO: 376 |
| TPP-23375 | Recombi Variant of TPP-15370 | Heavy Chain | PRT | SEQ ID NO: 377 |
| TPP-23375 | Recombi Variant of TPP-15370 | Light Chain | PRT | SEQ ID NO: 378 |
| TPP-23375 | Recombi Variant of TPP-15370 | Heavy Chain | DNA | SEQ ID NO: 379 |
| TPP-23375 | Recombi Variant of TPP-15370 | Light Chain | DNA | SEQ ID NO: 380 |
| TPP-25064 | Recombi Variant of TPP-15374 | VH | PRT | SEQ ID NO: 381 |
| TPP-25064 | Recombi Variant of TPP-15374 | HCDR1 | PRT | SEQ ID NO: 382 |
| TPP-25064 | Recombi Variant of TPP-15374 | HCDR2 | PRT | SEQ ID NO: 383 |
| TPP-25064 | Recombi Variant of TPP-15374 | HCDR3 | PRT | SEQ ID NO: 384 |
| TPP-25064 | Recombi Variant of TPP-15374 | VL | PRT | SEQ ID NO: 385 |
| TPP-25064 | Recombi Variant of TPP-15374 | LCDR1 | PRT | SEQ ID NO: 386 |
| TPP-25064 | Recombi Variant of TPP-15374 | LCDR2 | PRT | SEQ ID NO: 387 |
| TPP-25064 | Recombi Variant of TPP-15374 | LCDR3 | PRT | SEQ ID NO: 388 |
| TPP-25064 | Recombi Variant of TPP-15374 | VH | DNA | SEQ ID NO: 389 |
| TPP-25064 | Recombi Variant of TPP-15374 | HCDR1 | DNA | SEQ ID NO: 390 |
| TPP-25064 | Recombi Variant of TPP-15374 | HCDR2 | DNA | SEQ ID NO: 391 |
| TPP-25064 | Recombi Variant of TPP-15374 | HCDR3 | DNA | SEQ ID NO: 392 |
| TPP-25064 | Recombi Variant of TPP-15374 | VL | DNA | SEQ ID NO: 393 |
| TPP-25064 | Recombi Variant of TPP-15374 | LCDR1 | DNA | SEQ ID NO: 394 |
| TPP-25064 | Recombi Variant of TPP-15374 | LCDR2 | DNA | SEQ ID NO: 395 |
| TPP-25064 | Recombi Variant of TPP-15374 | LCDR3 | DNA | SEQ ID NO: 396 |
| TPP-25064 | Recombi Variant of TPP-15374 | Heavy Chain | PRT | SEQ ID NO: 397 |
| TPP-25064 | Recombi Variant of TPP-15374 | Light Chain | PRT | SEQ ID NO: 398 |
| TPP-25064 | Recombi Variant of TPP-15374 | Heavy Chain | DNA | SEQ ID NO: 399 |
| TPP-25064 | Recombi Variant of TPP-15374 | Light Chain | DNA | SEQ ID NO: 400 |
| TPP-25224 | Recombi Variant of TPP-15374 | VH | PRT | SEQ ID NO: 401 |
| TPP-25224 | Recombi Variant of TPP-15374 | HCDR1 | PRT | SEQ ID NO: 402 |
| TPP-25224 | Recombi Variant of TPP-15374 | HCDR2 | PRT | SEQ ID NO: 403 |
| TPP-25224 | Recombi Variant of TPP-15374 | HCDR3 | PRT | SEQ ID NO: 404 |
| TPP-25224 | Recombi Variant of TPP-15374 | VL | PRT | SEQ ID NO: 405 |
| TPP-25224 | Recombi Variant of TPP-15374 | LCDR1 | PRT | SEQ ID NO: 406 |
| TPP-25224 | Recombi Variant of TPP-15374 | LCDR2 | PRT | SEQ ID NO: 407 |
| TPP-25224 | Recombi Variant of TPP-15374 | LCDR3 | PRT | SEQ ID NO: 408 |
| TPP-25224 | Recombi Variant of TPP-15374 | VH | DNA | SEQ ID NO: 409 |
| TPP-25224 | Recombi Variant of TPP-15374 | HCDR1 | DNA | SEQ ID NO: 410 |
| TPP-25224 | Recombi Variant of TPP-15374 | HCDR2 | DNA | SEQ ID NO: 411 |
| TPP-25224 | Recombi Variant of TPP-15374 | HCDR3 | DNA | SEQ ID NO: 412 |
| TPP-25224 | Recombi Variant of TPP-15374 | VL | DNA | SEQ ID NO: 413 |
| TPP-25224 | Recombi Variant of TPP-15374 | LCDR1 | DNA | SEQ ID NO: 414 |
| TPP-25224 | Recombi Variant of TPP-15374 | LCDR2 | DNA | SEQ ID NO: 415 |
| TPP-25224 | Recombi Variant of TPP-15374 | LCDR3 | DNA | SEQ ID NO: 416 |
| TPP-25224 | Recombi Variant of TPP-15374 | Heavy Chain | PRT | SEQ ID NO: 417 |
| TPP-25224 | Recombi Variant of TPP-15374 | Light Chain | PRT | SEQ ID NO: 418 |
| TPP-25224 | Recombi Variant of TPP-15374 | Heavy Chain | DNA | SEQ ID NO: 419 |
| TPP-25224 | Recombi Variant of TPP-15374 | Light Chain | DNA | SEQ ID NO: 420 |
| TPP-25248 | Recombi Variant of TPP-15374 | VH | PRT | SEQ ID NO: 421 |
| TPP-25248 | Recombi Variant of TPP-15374 | HCDR1 | PRT | SEQ ID NO: 422 |
| TPP-25248 | Recombi Variant of TPP-15374 | HCDR2 | PRT | SEQ ID NO: 423 |
| TPP-25248 | Recombi Variant of TPP-15374 | HCDR3 | PRT | SEQ ID NO: 424 |
| TPP-25248 | Recombi Variant of TPP-15374 | VL | PRT | SEQ ID NO: 425 |
| TPP-25248 | Recombi Variant of TPP-15374 | LCDR1 | PRT | SEQ ID NO: 426 |
| TPP-25248 | Recombi Variant of TPP-15374 | LCDR2 | PRT | SEQ ID NO: 427 |
| TPP-25248 | Recombi Variant of TPP-15374 | LCDR3 | PRT | SEQ ID NO: 428 |
| TPP-25248 | Recombi Variant of TPP-15374 | VH | DNA | SEQ ID NO: 429 |
| TPP-25248 | Recombi Variant of TPP-15374 | HCDR1 | DNA | SEQ ID NO: 430 |
| TPP-25248 | Recombi Variant of TPP-15374 | HCDR2 | DNA | SEQ ID NO: 431 |
| TPP-25248 | Recombi Variant of TPP-15374 | HCDR3 | DNA | SEQ ID NO: 432 |
| TPP-25248 | Recombi Variant of TPP-15374 | VL | DNA | SEQ ID NO: 433 |
| TPP-25248 | Recombi Variant of TPP-15374 | LCDR1 | DNA | SEQ ID NO: 434 |
| TPP-25248 | Recombi Variant of TPP-15374 | LCDR2 | DNA | SEQ ID NO: 435 |
| TPP-25248 | Recombi Variant of TPP-15374 | LCDR3 | DNA | SEQ ID NO: 436 |
| TPP-25248 | Recombi Variant of TPP-15374 | Heavy Chain | PRT | SEQ ID NO: 437 |
| TPP-25248 | Recombi Variant of TPP-15374 | Light Chain | PRT | SEQ ID NO: 438 |
| TPP-25248 | Recombi Variant of TPP-15374 | Heavy Chain | DNA | SEQ ID NO: 439 |
| TPP-25248 | Recombi Variant of TPP-15374 | Light Chain | DNA | SEQ ID NO: 440 |
| TPP-25255 | Recombi Variant of TPP-15374 | VH | PRT | SEQ ID NO: 441 |
| TPP-25255 | Recombi Variant of TPP-15374 | HCDR1 | PRT | SEQ ID NO: 442 |
| TPP-25255 | Recombi Variant of TPP-15374 | HCDR2 | PRT | SEQ ID NO: 443 |
| TPP-25255 | Recombi Variant of TPP-15374 | HCDR3 | PRT | SEQ ID NO: 444 |
| TPP-25255 | Recombi Variant of TPP-15374 | VL | PRT | SEQ ID NO: 445 |
| TPP-25255 | Recombi Variant of TPP-15374 | LCDR1 | PRT | SEQ ID NO: 446 |
| TPP-25255 | Recombi Variant of TPP-15374 | LCDR2 | PRT | SEQ ID NO: 447 |
| TPP-25255 | Recombi Variant of TPP-15374 | LCDR3 | PRT | SEQ ID NO: 448 |
| TPP-25255 | Recombi Variant of TPP-15374 | VH | DNA | SEQ ID NO: 449 |
| TPP-25255 | Recombi Variant of TPP-15374 | HCDR1 | DNA | SEQ ID NO: 450 |
| TPP-25255 | Recombi Variant of TPP-15374 | HCDR2 | DNA | SEQ ID NO: 451 |
| TPP-25255 | Recombi Variant of TPP-15374 | HCDR3 | DNA | SEQ ID NO: 452 |
| TPP-25255 | Recombi Variant of TPP-15374 | VL | DNA | SEQ ID NO: 453 |
| TPP-25255 | Recombi Variant of TPP-15374 | LCDR1 | DNA | SEQ ID NO: 454 |
| TPP-25255 | Recombi Variant of TPP-15374 | LCDR2 | DNA | SEQ ID NO: 455 |
| TPP-25255 | Recombi Variant of TPP-15374 | LCDR3 | DNA | SEQ ID NO: 456 |
| TPP-25255 | Recombi Variant of TPP-15374 | Heavy Chain | PRT | SEQ ID NO: 457 |
| TPP-25255 | Recombi Variant of TPP-15374 | Light Chain | PRT | SEQ ID NO: 458 |
| TPP-25255 | Recombi Variant of TPP-15374 | Heavy Chain | DNA | SEQ ID NO: 459 |
| TPP-25255 | Recombi Variant of TPP-15374 | Light Chain | DNA | SEQ ID NO: 460 |
| TPP-25256 | Recombi Variant of TPP-15374 | VH | PRT | SEQ ID NO: 461 |
| TPP-25256 | Recombi Variant of TPP-15374 | HCDR1 | PRT | SEQ ID NO: 462 |
| TPP-25256 | Recombi Variant of TPP-15374 | HCDR2 | PRT | SEQ ID NO: 463 |
| TPP-25256 | Recombi Variant of TPP-15374 | HCDR3 | PRT | SEQ ID NO: 464 |
| TPP-25256 | Recombi Variant of TPP-15374 | VL | PRT | SEQ ID NO: 465 |
| TPP-25256 | Recombi Variant of TPP-15374 | LCDR1 | PRT | SEQ ID NO: 466 |
| TPP-25256 | Recombi Variant of TPP-15374 | LCDR2 | PRT | SEQ ID NO: 467 |
| TPP-25256 | Recombi Variant of TPP-15374 | LCDR3 | PRT | SEQ ID NO: 468 |
| TPP-25256 | Recombi Variant of TPP-15374 | VH | DNA | SEQ ID NO: 469 |
| TPP-25256 | Recombi Variant of TPP-15374 | HCDR1 | DNA | SEQ ID NO: 470 |
| TPP-25256 | Recombi Variant of TPP-15374 | HCDR2 | DNA | SEQ ID NO: 471 |
| TPP-25256 | Recombi Variant of TPP-15374 | HCDR3 | DNA | SEQ ID NO: 472 |
| TPP-25256 | Recombi Variant of TPP-15374 | VL | DNA | SEQ ID NO: 473 |
| TPP-25256 | Recombi Variant of TPP-15374 | LCDR1 | DNA | SEQ ID NO: 474 |
| TPP-25256 | Recombi Variant of TPP-15374 | LCDR2 | DNA | SEQ ID NO: 475 |
| TPP-25256 | Recombi Variant of TPP-15374 | LCDR3 | DNA | SEQ ID NO: 476 |
| TPP-25256 | Recombi Variant of TPP-15374 | Heavy Chain | PRT | SEQ ID NO: 477 |
| TPP-25256 | Recombi Variant of TPP-15374 | Light Chain | PRT | SEQ ID NO: 478 |
| TPP-25256 | Recombi Variant of TPP-15374 | Heavy Chain | DNA | SEQ ID NO: 479 |
| TPP-25256 | Recombi Variant of TPP-15374 | Light Chain | DNA | SEQ ID NO: 480 |
| TPP-25257 | Recombi Variant of TPP-15374 | VH | PRT | SEQ ID NO: 481 |
| TPP-25257 | Recombi Variant of TPP-15374 | HCDR1 | PRT | SEQ ID NO: 482 |
| TPP-25257 | Recombi Variant of TPP-15374 | HCDR2 | PRT | SEQ ID NO: 483 |
| TPP-25257 | Recombi Variant of TPP-15374 | HCDR3 | PRT | SEQ ID NO: 484 |
| TPP-25257 | Recombi Variant of TPP-15374 | VL | PRT | SEQ ID NO: 485 |
| TPP-25257 | Recombi Variant of TPP-15374 | LCDR1 | PRT | SEQ ID NO: 486 |
| TPP-25257 | Recombi Variant of TPP-15374 | LCDR2 | PRT | SEQ ID NO: 487 |
| TPP-25257 | Recombi Variant of TPP-15374 | LCDR3 | PRT | SEQ ID NO: 488 |
| TPP-25257 | Recombi Variant of TPP-15374 | VH | DNA | SEQ ID NO: 489 |
| TPP-25257 | Recombi Variant of TPP-15374 | HCDR1 | DNA | SEQ ID NO: 490 |
| TPP-25257 | Recombi Variant of TPP-15374 | HCDR2 | DNA | SEQ ID NO: 491 |
| TPP-25257 | Recombi Variant of TPP-15374 | HCDR3 | DNA | SEQ ID NO: 492 |
| TPP-25257 | Recombi Variant of TPP-15374 | VL | DNA | SEQ ID NO: 493 |
| TPP-25257 | Recombi Variant of TPP-15374 | LCDR1 | DNA | SEQ ID NO: 494 |
| TPP-25257 | Recombi Variant of TPP-15374 | LCDR2 | DNA | SEQ ID NO: 495 |
| TPP-25257 | Recombi Variant of TPP-15374 | LCDR3 | DNA | SEQ ID NO: 496 |
| TPP-25257 | Recombi Variant of TPP-15374 | Heavy Chain | PRT | SEQ ID NO: 497 |
| TPP-25257 | Recombi Variant of TPP-15374 | Light Chain | PRT | SEQ ID NO: 498 |
| TPP-25257 | Recombi Variant of TPP-15374 | Heavy Chain | DNA | SEQ ID NO: 499 |
| TPP-25257 | Recombi Variant of TPP-15374 | Light Chain | DNA | SEQ ID NO: 500 |
| TPP-25448 | Recombi Variant of TPP-15374 | VH | PRT | SEQ ID NO: 501 |
| TPP-25448 | Recombi Variant of TPP-15374 | HCDR1 | PRT | SEQ ID NO: 502 |
| TPP-25448 | Recombi Variant of TPP-15374 | HCDR2 | PRT | SEQ ID NO: 503 |
| TPP-25448 | Recombi Variant of TPP-15374 | HCDR3 | PRT | SEQ ID NO: 504 |
| TPP-25448 | Recombi Variant of TPP-15374 | VL | PRT | SEQ ID NO: 505 |
| TPP-25448 | Recombi Variant of TPP-15374 | LCDR1 | PRT | SEQ ID NO: 506 |
| TPP-25448 | Recombi Variant of TPP-15374 | LCDR2 | PRT | SEQ ID NO: 507 |
| TPP-25448 | Recombi Variant of TPP-15374 | LCDR3 | PRT | SEQ ID NO: 508 |
| TPP-25448 | Recombi Variant of TPP-15374 | VH | DNA | SEQ ID NO: 509 |
| TPP-25448 | Recombi Variant of TPP-15374 | HCDR1 | DNA | SEQ ID NO: 510 |
| TPP-25448 | Recombi Variant of TPP-15374 | HCDR2 | DNA | SEQ ID NO: 511 |
| TPP-25448 | Recombi Variant of TPP-15374 | HCDR3 | DNA | SEQ ID NO: 512 |
| TPP-25448 | Recombi Variant of TPP-15374 | VL | DNA | SEQ ID NO: 513 |
| TPP-25448 | Recombi Variant of TPP-15374 | LCDR1 | DNA | SEQ ID NO: 514 |
| TPP-25448 | Recombi Variant of TPP-15374 | LCDR2 | DNA | SEQ ID NO: 515 |
| TPP-25448 | Recombi Variant of TPP-15374 | LCDR3 | DNA | SEQ ID NO: 516 |
| TPP-25448 | Recombi Variant of TPP-15374 | Heavy Chain | PRT | SEQ ID NO: 517 |
| TPP-25448 | Recombi Variant of TPP-15374 | Light Chain | PRT | SEQ ID NO: 518 |
| TPP-25448 | Recombi Variant of TPP-15374 | Heavy Chain | DNA | SEQ ID NO: 519 |
| TPP-25448 | Recombi Variant of TPP-15374 | Light Chain | DNA | SEQ ID NO: 520 |
| TPP-25497 | Recombi Variant of TPP-15374 | VH | PRT | SEQ ID NO: 521 |
| TPP-25497 | Recombi Variant of TPP-15374 | HCDR1 | PRT | SEQ ID NO: 522 |
| TPP-25497 | Recombi Variant of TPP-15374 | HCDR2 | PRT | SEQ ID NO: 523 |
| TPP-25497 | Recombi Variant of TPP-15374 | HCDR3 | PRT | SEQ ID NO: 524 |
| TPP-25497 | Recombi Variant of TPP-15374 | VL | PRT | SEQ ID NO: 525 |
| TPP-25497 | Recombi Variant of TPP-15374 | LCDR1 | PRT | SEQ ID NO: 526 |
| TPP-25497 | Recombi Variant of TPP-15374 | LCDR2 | PRT | SEQ ID NO: 527 |
| TPP-25497 | Recombi Variant of TPP-15374 | LCDR3 | PRT | SEQ ID NO: 528 |
| TPP-25497 | Recombi Variant of TPP-15374 | VH | DNA | SEQ ID NO: 529 |
| TPP-25497 | Recombi Variant of TPP-15374 | HCDR1 | DNA | SEQ ID NO: 530 |
| TPP-25497 | Recombi Variant of TPP-15374 | HCDR2 | DNA | SEQ ID NO: 531 |
| TPP-25497 | Recombi Variant of TPP-15374 | HCDR3 | DNA | SEQ ID NO: 532 |
| TPP-25497 | Recombi Variant of TPP-15374 | VL | DNA | SEQ ID NO: 533 |
| TPP-25497 | Recombi Variant of TPP-15374 | LCDR1 | DNA | SEQ ID NO: 534 |
| TPP-25497 | Recombi Variant of TPP-15374 | LCDR2 | DNA | SEQ ID NO: 535 |
| TPP-25497 | Recombi Variant of TPP-15374 | LCDR3 | DNA | SEQ ID NO: 536 |
| TPP-25497 | Recombi Variant of TPP-15374 | Heavy Chain | PRT | SEQ ID NO: 537 |
| TPP-25497 | Recombi Variant of TPP-15374 | Light Chain | PRT | SEQ ID NO: 538 |
| TPP-25497 | Recombi Variant of TPP-15374 | Heavy Chain | DNA | SEQ ID NO: 539 |
| TPP-25497 | Recombi Variant of TPP-15374 | Light Chain | DNA | SEQ ID NO: 540 |
| TPP-25655 | Recombi Variant of TPP-15374 | VH | PRT | SEQ ID NO: 541 |
| TPP-25655 | Recombi Variant of TPP-15374 | HCDR1 | PRT | SEQ ID NO: 542 |
| TPP-25655 | Recombi Variant of TPP-15374 | HCDR2 | PRT | SEQ ID NO: 543 |
| TPP-25655 | Recombi Variant of TPP-15374 | HCDR3 | PRT | SEQ ID NO: 544 |
| TPP-25655 | Recombi Variant of TPP-15374 | VL | PRT | SEQ ID NO: 545 |
| TPP-25655 | Recombi Variant of TPP-15374 | LCDR1 | PRT | SEQ ID NO: 546 |
| TPP-25655 | Recombi Variant of TPP-15374 | LCDR2 | PRT | SEQ ID NO: 547 |
| TPP-25655 | Recombi Variant of TPP-15374 | LCDR3 | PRT | SEQ ID NO: 548 |
| TPP-25655 | Recombi Variant of TPP-15374 | VH | DNA | SEQ ID NO: 549 |
| TPP-25655 | Recombi Variant of TPP-15374 | HCDR1 | DNA | SEQ ID NO: 550 |
| TPP-25655 | Recombi Variant of TPP-15374 | HCDR2 | DNA | SEQ ID NO: 551 |
| TPP-25655 | Recombi Variant of TPP-15374 | HCDR3 | DNA | SEQ ID NO: 552 |
| TPP-25655 | Recombi Variant of TPP-15374 | VL | DNA | SEQ ID NO: 553 |
| TPP-25655 | Recombi Variant of TPP-15374 | LCDR1 | DNA | SEQ ID NO: 554 |
| TPP-25655 | Recombi Variant of TPP-15374 | LCDR2 | DNA | SEQ ID NO: 555 |
| TPP-25655 | Recombi Variant of TPP-15374 | LCDR3 | DNA | SEQ ID NO: 556 |
| TPP-25655 | Recombi Variant of TPP-15374 | Heavy Chain | PRT | SEQ ID NO: 557 |
| TPP-25655 | Recombi Variant of TPP-15374 | Light Chain | PRT | SEQ ID NO: 558 |
| TPP-25655 | Recombi Variant of TPP-15374 | Heavy Chain | DNA | SEQ ID NO: 559 |
| TPP-25655 | Recombi Variant of TPP-15374 | Light Chain | DNA | SEQ ID NO: 560 |
| TPP-26111 | Recombi Variant of TPP-15374 | VH | PRT | SEQ ID NO: 561 |
| TPP-26111 | Recombi Variant of TPP-15374 | HCDR1 | PRT | SEQ ID NO: 562 |
| TPP-26111 | Recombi Variant of TPP-15374 | HCDR2 | PRT | SEQ ID NO: 563 |
| TPP-26111 | Recombi Variant of TPP-15374 | HCDR3 | PRT | SEQ ID NO: 564 |
| TPP-26111 | Recombi Variant of TPP-15374 | VL | PRT | SEQ ID NO: 565 |
| TPP-26111 | Recombi Variant of TPP-15374 | LCDR1 | PRT | SEQ ID NO: 566 |
| TPP-26111 | Recombi Variant of TPP-15374 | LCDR2 | PRT | SEQ ID NO: 567 |
| TPP-26111 | Recombi Variant of TPP-15374 | LCDR3 | PRT | SEQ ID NO: 568 |
| TPP-26111 | Recombi Variant of TPP-15374 | VH | DNA | SEQ ID NO: 569 |
| TPP-26111 | Recombi Variant of TPP-15374 | HCDR1 | DNA | SEQ ID NO: 570 |
| TPP-26111 | Recombi Variant of TPP-15374 | HCDR2 | DNA | SEQ ID NO: 571 |
| TPP-26111 | Recombi Variant of TPP-15374 | HCDR3 | DNA | SEQ ID NO: 572 |
| TPP-26111 | Recombi Variant of TPP-15374 | VL | DNA | SEQ ID NO: 573 |
| TPP-26111 | Recombi Variant of TPP-15374 | LCDR1 | DNA | SEQ ID NO: 574 |
| TPP-26111 | Recombi Variant of TPP-15374 | LCDR2 | DNA | SEQ ID NO: 575 |
| TPP-26111 | Recombi Variant of TPP-15374 | LCDR3 | DNA | SEQ ID NO: 576 |
| TPP-26111 | Recombi Variant of TPP-15374 | Heavy Chain | PRT | SEQ ID NO: 577 |
| TPP-26111 | Recombi Variant of TPP-15374 | Light Chain | PRT | SEQ ID NO: 578 |
| TPP-26111 | Recombi Variant of TPP-15374 | Heavy Chain | DNA | SEQ ID NO: 579 |
| TPP-26111 | Recombi Variant of TPP-15374 | Light Chain | DNA | SEQ ID NO: 580 |
| TPP-13211 | huSema3a_FXaFc | Chain 1 | PRT | SEQ ID NO: 581 |
| TPP-19068 | human Sema3a_FXaHis6 | Chain 1 | PRT | SEQ ID NO: 582 |
| TPP-19069 | mouse Sema3a_FXaHis6 | Chain 1 | PRT | SEQ ID NO: 583 |
| TPP-19120 | rat-Sema3a_FXaHis6 | Chain 1 | PRT | SEQ ID NO: 584 |
| TPP-19121 | dog-Sema3a_FXaHis6 | Chain 1 | PRT | SEQ ID NO: 585 |
| TPP-19122 | cyno-Sema3a_FXaHis6 | Chain 1 | PRT | SEQ ID NO: 586 |
| TPP-20176 | pigSema3A_FXaHis6 | Chain 1 | PRT | SEQ ID NO: 587 |
| TPP-30788 | Böhringer (BI) Clone I | VH | PRT | SEQ ID NO: 800 |
| TPP-30788 | Böhringer (BI) Clone I | HCDR1 | PRT | SEQ ID NO: 801 |
| TPP-30788 | Böhringer (BI) Clone I | HCDR2 | PRT | SEQ ID NO: 802 |
| TPP-30788 | Böhringer (BI) Clone I | HCDR3 | PRT | SEQ ID NO: 803 |
| TPP-30788 | Böhringer (BI) Clone I | VL | PRT | SEQ ID NO: 804 |
| TPP-30788 | Böhringer (BI) Clone I | LCDR1 | PRT | SEQ ID NO: 805 |
| TPP-30788 | Böhringer (BI) Clone I | LCDR2 | PRT | SEQ ID NO: 806 |
| TPP-30788 | Böhringer (BI) Clone I | LCDR3 | PRT | SEQ ID NO: 807 |
| TPP-30788 | Böhringer (BI) Clone I | VH | DNA | SEQ ID NO: 808 |
| TPP-30788 | Böhringer (BI) Clone I | VL | DNA | SEQ ID NO: 809 |
| TPP-30788 | Böhringer (BI) Clone I | Heavy Chain | PRT | SEQ ID NO: 810 |
| TPP-30788 | Böhringer (BI) Clone I | Light Chain | PRT | SEQ ID NO: 811 |
| TPP-30788 | Böhringer (BI) Clone I | Heavy Chain | DNA | SEQ ID NO: 812 |
| TPP-30788 | Böhringer (BI) Clone I | Light Chain | DNA | SEQ ID NO: 813 |
| TPP-30789 | Böhringer (BI) Clone II | VH | PRT | SEQ ID NO: 814 |
| TPP-30789 | Böhringer (BI) Clone II | HCDR1 | PRT | SEQ ID NO: 815 |
| TPP-30789 | Böhringer (BI) Clone II | HCDR2 | PRT | SEQ ID NO: 816 |
| TPP-30789 | Böhringer (BI) Clone II | HCDR3 | PRT | SEQ ID NO: 817 |
| TPP-30789 | Böhringer (BI) Clone II | VL | PRT | SEQ ID NO: 818 |
| TPP-30789 | Böhringer (BI) Clone II | LCDR1 | PRT | SEQ ID NO: 819 |
| TPP-30789 | Böhringer (BI) Clone II | LCDR2 | PRT | SEQ ID NO: 820 |
| TPP-30789 | Böhringer (BI) Clone II | LCDR3 | PRT | SEQ ID NO: 821 |
| TPP-30789 | Böhringer (BI) Clone II | VH | DNA | SEQ ID NO: 822 |
| TPP-30789 | Böhringer (BI) Clone II | VL | DNA | SEQ ID NO: 823 |
| TPP-30789 | Böhringer (BI) Clone II | Heavy Chain | PRT | SEQ ID NO: 824 |
| TPP-30789 | Böhringer (BI) Clone II | Light Chain | PRT | SEQ ID NO: 825 |
| TPP-30789 | Böhringer (BI) Clone II | Heavy Chain | DNA | SEQ ID NO: 826 |
| TPP-30789 | Böhringer (BI) Clone II | Light Chain | DNA | SEQ ID NO: 827 |
| TPP-30790 | Böhringer (BI) Clone III | VH | PRT | SEQ ID NO: 828 |
| TPP-30790 | Böhringer (BI) Clone III | HCDR1 | PRT | SEQ ID NO: 829 |
| TPP-30790 | Böhringer (BI) Clone III | HCDR2 | PRT | SEQ ID NO: 830 |
| TPP-30790 | Böhringer (BI) Clone III | HCDR3 | PRT | SEQ ID NO: 831 |
| TPP-30790 | Böhringer (BI) Clone III | VL | PRT | SEQ ID NO: 832 |
| TPP-30790 | Böhringer (BI) Clone III | LCDR1 | PRT | SEQ ID NO: 833 |
| TPP-30790 | Böhringer (BI) Clone III | LCDR2 | PRT | SEQ ID NO: 834 |
| TPP-30790 | Böhringer (BI) Clone III | LCDR3 | PRT | SEQ ID NO: 835 |
| TPP-30790 | Böhringer (BI) Clone III | VH | DNA | SEQ ID NO: 836 |
| TPP-30790 | Böhringer (BI) Clone III | VL | DNA | SEQ ID NO: 837 |
| TPP-30790 | Böhringer (BI) Clone III | Heavy Chain | PRT | SEQ ID NO: 838 |
| TPP-30790 | Böhringer (BI) Clone III | Light Chain | PRT | SEQ ID NO: 839 |
| TPP-30790 | Böhringer (BI) Clone III | Heavy Chain | DNA | SEQ ID NO: 840 |
| TPP-30790 | Böhringer (BI) Clone III | Light Chain | DNA | SEQ ID NO: 841 |
| TPP-30791 | Böhringer (BI) Clone IV | VH | PRT | SEQ ID NO: 842 |
| TPP-30791 | Böhringer (BI) Clone IV | HCDR1 | PRT | SEQ ID NO: 843 |
| TPP-30791 | Böhringer (BI) Clone IV | HCDR2 | PRT | SEQ ID NO: 844 |
| TPP-30791 | Böhringer (BI) Clone IV | HCDR3 | PRT | SEQ ID NO: 845 |
| TPP-30791 | Böhringer (BI) Clone IV | VL | PRT | SEQ ID NO: 846 |
| TPP-30791 | Böhringer (BI) Clone IV | LCDR1 | PRT | SEQ ID NO: 847 |
| TPP-30791 | Böhringer (BI) Clone IV | LCDR2 | PRT | SEQ ID NO: 848 |
| TPP-30791 | Böhringer (BI) Clone IV | LCDR3 | PRT | SEQ ID NO: 849 |
| TPP-30791 | Böhringer (BI) Clone IV | VH | DNA | SEQ ID NO: 850 |
| TPP-30791 | Böhringer (BI) Clone IV | VL | DNA | SEQ ID NO: 851 |
| TPP-30791 | Böhringer (BI) Clone IV | Heavy Chain | PRT | SEQ ID NO: 852 |
| TPP-30791 | Böhringer (BI) Clone IV | Light Chain | PRT | SEQ ID NO: 853 |
| TPP-30791 | Böhringer (BI) Clone IV | Heavy Chain | DNA | SEQ ID NO: 854 |
| TPP-30791 | Böhringer (BI) Clone IV | Light Chain | DNA | SEQ ID NO: 855 |
| TPP-30792 | 3H4 (Ramot) Clon I | VH | PRT | SEQ ID NO: 856 |
| TPP-30792 | 3H4 (Ramot) Clon I | HCDR1 | PRT | SEQ ID NO: 857 |
| TPP-30792 | 3H4 (Ramot) Clon I | HCDR2 | PRT | SEQ ID NO: 858 |
| TPP-30792 | 3H4 (Ramot) Clon I | HCDR3 | PRT | SEQ ID NO: 859 |
| TPP-30792 | 3H4 (Ramot) Clon I | VL | PRT | SEQ ID NO: 860 |
| TPP-30792 | 3H4 (Ramot) Clon I | LCDR1 | PRT | SEQ ID NO: 861 |
| TPP-30792 | 3H4 (Ramot) Clon I | LCDR2 | PRT | SEQ ID NO: 862 |
| TPP-30792 | 3H4 (Ramot) Clon I | LCDR3 | PRT | SEQ ID NO: 863 |
| TPP-30792 | 3H4 (Ramot) Clon I | VH | DNA | SEQ ID NO: 864 |
| TPP-30792 | 3H4 (Ramot) Clon I | VL | DNA | SEQ ID NO: 865 |
| TPP-30792 | 3H4 (Ramot) Clon I | Heavy Chain | PRT | SEQ ID NO: 866 |
| TPP-30792 | 3H4 (Ramot) Clon I | Light Chain | PRT | SEQ ID NO: 867 |
| TPP-30792 | 3H4 (Ramot) Clon I | Heavy Chain | DNA | SEQ ID NO: 868 |
| TPP-30792 | 3H4 (Ramot) Clon I | Light Chain | DNA | SEQ ID NO: 869 |
| TPP-31357 | Fab of 3H4 Univ Ramot | VH | PRT | SEQ ID NO: 870 |
| TPP-31357 | Fab of 3H4 Univ Ramot | HCDR1 | PRT | SEQ ID NO: 871 |
| TPP-31357 | Fab of 3H4 Univ Ramot | HCDR2 | PRT | SEQ ID NO: 872 |
| TPP-31357 | Fab of 3H4 Univ Ramot | HCDR3 | PRT | SEQ ID NO: 873 |
| TPP-31357 | Fab of 3H4 Univ Ramot | VL | PRT | SEQ ID NO: 874 |
| TPP-31357 | Fab of 3H4 Univ Ramot | LCDR1 | PRT | SEQ ID NO: 875 |
| TPP-31357 | Fab of 3H4 Univ Ramot | LCDR2 | PRT | SEQ ID NO: 876 |
| TPP-31357 | Fab of 3H4 Univ Ramot | LCDR3 | PRT | SEQ ID NO: 877 |
| TPP-31357 | Fab of 3H4 Univ Ramot | VH | DNA | SEQ ID NO: 878 |
| TPP-31357 | Fab of 3H4 Univ Ramot | VL | DNA | SEQ ID NO: 879 |
| TPP-31357 | Fab of 3H4 Univ Ramot | Heavy Chain | PRT | SEQ ID NO: 880 |
| TPP-31357 | Fab of 3H4 Univ Ramot | Light Chain | PRT | SEQ ID NO: 881 |
| TPP-31357 | Fab of 3H4 Univ Ramot | Heavy Chain | DNA | SEQ ID NO: 882 |
| TPP-31357 | Fab of 3H4 Univ Ramot | Light Chain | DNA | SEQ ID NO: 883 |

TABLE 1A
Corresponding amino acid sequences and nucleic acid sequences of antibodies according
to the present disclosure mentioned in table 1 under the respective SEQ IDs. SEQ ID 581 to 587 being
the corresponding Sema3A protein sequences from Homo sapiens (SEQ ID 581, 582), Mus Musculus
(SEQ ID 583), Rattus norvegicus (SEQ ID 584), Canis lupus familiaris (SEQ ID 585), Macaca
fascicularis (SEQ ID 586), Sus scrofa (SEQ ID 587).

| SEQ | SEQ | |
|---|---|---|
| ID No | Type | SEQUENCE |
| 1 | PRT | EVQLLESGGGLVQPGGSLRLSCAASGFTFSSYPMGWVRQAPGKGLEWV |
| | | AGIDDDGDSDTRYAPAVKGRATISRDNSKNTVYLQMNSLRAEDTAVYY |
| | | CAKHTGIGANSAGSIDAWGQGTLVTVSS |
| 2 | PRT | SYPMG |
| 3 | PRT | GIDDDGDSDTRYAPAVKG |
| 4 | PRT | HTGIGANSAGSIDA |
| 5 | PRT | SYELTQPPSVSVSPGQTARITCSGGGSYTGSYYYGWYQQKPGQAPVTVI |
| | | YYNNKRPSDIPERFSGSLSGTTNTLTISGVQAEDEADYYCGSADNSGDAF |
| | | GTGTKVTVL |
| 6 | PRT | SGGGSYTGSYYYG |
| 7 | PRT | YNNKRPS |
| 8 | PRT | GSADNSGDA |
| 9 | DNA | GAAGTTCAGCTGCTGGAATCTGGCGGCGGACTGGTTCAACCTGGCGG |
| | | ATCTCTGAGACTGAGCTGTGCCGCCAGCGGCTTCACCTTTAGCAGCT |
| | | ATCCTATGGGCTGGGTCCGACAGGCCCCTGGCAAAGGACTTGAATGG |
| | | GTGGCCGGCATCGACGACGATGGCGATAGCGATACAAGATACGCCC |
| | | CTGCCGTGAAGGGCAGAGCCACCATCTCCAGAGACAACAGCAAGAA |
| | | CACCGTGTACCTGCAGATGAACAGCCTGAGAGCCGAGGACACCGCC |
| | | GTGTACTATTGTGCCAAGCACACAGGCATCGGCGCCAATTCTGCCGG |
| | | CTCTATTGATGCCTGGGGCCAGGGAACACTGGTCACAGTTTCTTCA |
| 10 | DNA | AGCTATCCTATGGGC |
| 11 | DNA | GGCATCGACGACGATGGCGATAGCGATACAAGATACGCCCCTGCCGT |
| | | GAAGGGC |
| 12 | DNA | CACACAGGCATCGGCGCCAATTCTGCCGGCTCTATTGATGCC |
| 13 | DNA | AGCTATGAGCTGACACAGCCTCCAAGCGTGTCCGTGTCTCCTGGACA |
| | | GACCGCCAGAATCACATGTAGCGGCGGAGGCAGCTACACCGGCAGC |
| | | TACTACTATGGCTGGTATCAGCAGAAGCCCGGACAGGCCCCTGTGAC |
| | | CGTGATCTACTACAACAACAAGCGGCCCAGCGACATCCCCGAGAGAT |
| | | TTTCTGGCTCTCTGAGCGGCACCACCAACACACTGACAATCTCTGGC |
| | | GTGCAGGCCGAGGACGAGGCCGATTACTATTGTGGCAGCGCCGATAA |
| | | TAGCGGCGACGCCTTTGGCACCGGCACCAAAGTTACAGTGCTA |
| 14 | DNA | AGCGGCGGAGGCAGCTACACCGGCAGCTACTACTATGGC |
| 15 | DNA | TACAACAACAAGCGGCCCAGC |
| 16 | DNA | GGCAGCGCCGATAATAGCGGCGACGCC |
| 17 | PRT | EVQLLESGGGLVQPGGSLRLSCAASGFTFSSYPMGWVRQAPGKGLEWV |
| | | AGIDDDGDSDTRYAPAVKGRATISRDNSKNTVYLQMNSLRAEDTAVYY |
| | | CAKHTGIGANSAGSIDAWGQGTLVTVSSASTKGPSVFPLAPSSKSTSGGT |
| | | AALGCLVKDYFPEPVTVSWNSGALTSGVHTFPAVLQSSGLYSLSSVVTV |
| | | PSSSLGTQTYICNVNHKPSNTKVDKKVEPKSCDKTHTCPPCPAPELLGGP |
| | | SVFLFPPKPKDTLMISRTPEVTCVVVDVSHEDPEVKFNWYVDGVEVHNA |
| | | KTKPREEQYNSTYRVVSVLTVLHQDWLNGKEYKCKVSNKALPAPIEKTI |
| | | SKAKGQPREPQVYTLPPSRDELTKNQVSLTCLVKGFYPSDIAVEWESNG |
| | | QPENNYKTTPPVLDSDGSFFLYSKLTVDKSRWQQGNVFSCSVMHEALH |
| | | NHYTQKSLSLSPG |
| 18 | PRT | SYELTQPPSVSVSPGQTARITCSGGGSYTGSYYYGWYQQKPGQAPVTVI |
| | | YYNNKRPSDIPERFSGSLSGTTNTLTISGVQAEDEADYYCGSADNSGDAF |
| | | GTGTKVTVLGQPKAAPSVTLFPPSSEELQANKATLVCLISDFYPGAVTVA |
| | | WKADSSPVKAGVETTTPSKQSNNKYAASSYLSLTPEQWKSHRSYSCQV |
| | | THEGSTVEKTVAPTECS |
| 19 | DNA | GAAGTTCAGCTGCTGGAATCTGGCGGCGGACTGGTTCAACCTGGCGG |
| | | ATCTCTGAGACTGAGCTGTGCCGCCAGCGGCTTCACCTTTAGCAGCT |
| | | ATCCTATGGGCTGGGTCCGACAGGCCCCTGGCAAAGGACTTGAATGG |
| | | GTGGCCGGCATCGACGACGATGGCGATAGCGATACAAGATACGCCC |
| | | CTGCCGTGAAGGGCAGAGCCACCATCTCCAGAGACAACAGCAAGAA |
| | | CACCGTGTACCTGCAGATGAACAGCCTGAGAGCCGAGGACACCGCC |
| | | GTGTACTATTGTGCCAAGCACACAGGCATCGGCGCCAATTCTGCCGG |
| | | CTCTATTGATGCCTGGGGCCAGGGAACACTGGTCACAGTTTCTTCAG |
| | | CCAGCACCAAGGGCCCCAGCGTGTTCCCTCTGGCCCCTAGCAGCAAG |
| | | AGCACATCTGGCGGAACAGCCGCCCTGGGCTGCCTCGTGAAGGACTA |
| | | CTTTCCCGAGCCCGTGACCGTGTCCTGGAACTCTGGCGCTCTGACAA |
| | | GCGGCGTGCACACCTTTCCAGCCGTGCTGCAGAGCAGCGGCCTGTAC |
| | | TCTCTGAGCAGCGTCGTGACAGTGCCCAGCAGCTCTCTGGGCACCCA |
| | | GACCTACATCTGCAACGTGAACCACAAGCCCAGCAACACCAAGGTG |
| | | GACAAGAAGGTGGAACCCAAGAGCTGCGACAAGACCCACACCTGTC |
| | | CCCCTTGTCCTGCCCCCGAACTGCTGGGAGGCCCTTCCGTGTTCCTGT |
| | | TCCCCCCAAAGCCCAAGGACACCCTGATGATCAGCCGGACCCCCGAA |
| | | GTGACCTGCGTGGTGGTGGATGTGTCCCACGAGGACCCTGAAGTGAA |
| | | GTTCAATTGGTACGTGGACGGCGTGGAAGTGCACAACGCCAAGACCA |
| | | AGCCTAGAGAGGAACAGTACAACAGCACCTACCGGGTGGTGTCCGT |
| | | GCTGACAGTGCTGCACCAGGACTGGCTGAACGGCAAAGAGTACAAG |
| | | TGCAAGGTGTCCAACAAGGCCCTGCCTGCCCCCATCGAGAAAACCAT |
| | | CAGCAAGGCCAAGGGCCAGCCCCGCGAACCCCAGGTGTACACACTG |
| | | CCCCCAAGCAGGGACGAGCTGACCAAGAACCAGGTGTCCCTGACCTG |
| | | TCTCGTGAAAGGCTTCTACCCCTCCGATATCGCCGTGGAATGGGAGA |
| | | GCAACGGCCAGCCCGAGAACAACTACAAGACCACCCCCCCTGTGCTG |
| | | GACAGCGACGGCTCATTCTTCCTGTACAGCAAGCTGACCGTGGACAA |
| | | GTCCCGGTGGCAGCAGGGCAACGTGTTCAGCTGCAGCGTGATGCACG |
| | | AGGCCCTGCACAACCACTACACCCAGAAGTCCCTGAGCCTGAGCCCT |
| | | GGC |
| 20 | DNA | AGCTATGAGCTGACACAGCCTCCAAGCGTGTCCGTGTCTCCTGGACA |
| | | GACCGCCAGAATCACATGTAGCGGCGGAGGCAGCTACACCGGCAGC |
| | | TACTACTATGGCTGGTATCAGCAGAAGCCCGGACAGGCCCCTGTGAC |
| | | CGTGATCTACTACAACAACAAGCGGCCCAGCGACATCCCCGAGAGAT |
| | | TTTCTGGCTCTCTGAGCGGCACCACCAACACACTGACAATCTCTGGC |
| | | GTGCAGGCCGAGGACGAGGCCGATTACTATTGTGGCAGCGCCGATAA |
| | | TAGCGGCGACGCCTTTGGCACCGGCACCAAAGTTACAGTGCTAGGCC |
| | | AGCCTAAAGCCGCCCCTAGCGTGACCCTGTTCCCTCCAAGCAGCGAG |
| | | GAACTGCAGGCCAACAAGGCCACCCTCGTGTGCCTGATCAGCGACTT |
| | | CTATCCTGGCGCCGTGACCGTGGCCTGGAAGGCCGATAGCTCTCCTG |
| | | TGAAGGCCGGCGTGGAAACCACCACCCCTAGCAAGCAGAGCAACAA |
| | | CAAATACGCCGCCAGCAGCTACCTGAGCCTGACCCCCGAGCAGTGGA |
| | | AGTCCCACAGATCCTACAGCTGCCAAGTGACCCACGAGGGCAGCACC |
| | | GTGGAAAAGACAGTGGCCCCTACCGAGTGCAGC |
| 21 | PRT | EVQLLESGGGLVQPGGSLRLSCAASGFTFSSYPMGWVRQAPGKGLEWV |
| | | AGIDDDGDSDTRYAPAVKGRATISRDNSKNTVYLQMNSLRAEDTAVYY |
| | | CAKHTGIGANSAGSIDAWGQGTLVTVSS |
| 22 | PRT | SYPMG |
| 23 | PRT | GIDDDGDSDTRYAPAVKG |
| 24 | PRT | HTGIGANSAGSIDA |
| 25 | PRT | SYELTQPPSVSVSPGQTARITCSGGGSYTGSYYYGWYQQKPGQAPVTVI |
| | | YYNNKRPSDIPERFSGSLSGTTNTLTISGVQAEDEADYYCGSADNSGDAF |
| | | GTGTKVTVL |
| 26 | PRT | SGGGSYTGSYYYG |
| 27 | PRT | YNNKRPS |
| 28 | PRT | GSADNSGDA |
| 29 | DNA | GAAGTTCAGCTGCTGGAATCTGGCGGCGGACTGGTTCAACCTGGCGG |
| | | ATCTCTGAGACTGAGCTGTGCCGCCAGCGGCTTCACCTTTAGCAGCT |
| | | ATCCTATGGGCTGGGTCCGACAGGCCCCTGGCAAAGGACTTGAATGG |
| | | GTGGCCGGCATCGACGACGATGGCGATAGCGATACAAGATACGCCC |
| | | CTGCCGTGAAGGGCAGAGCCACCATCTCCAGAGACAACAGCAAGAA |
| | | CACCGTGTACCTGCAGATGAACAGCCTGAGAGCCGAGGACACCGCC |
| | | GTGTACTATTGTGCCAAGCACACAGGCATCGGCGCCAATTCTGCCGG |
| | | CTCTATTGATGCCTGGGGCCAGGGAACACTGGTCACAGTTTCTTCA |
| 30 | DNA | AGCTATCCTATGGGC |
| 31 | DNA | GGCATCGACGACGATGGCGATAGCGATACAAGATACGCCCCTGCCGT |
| | | GAAGGGC |
| 32 | DNA | CACACAGGCATCGGCGCCAATTCTGCCGGCTCTATTGATGCC |
| 33 | DNA | AGCTATGAGCTGACACAGCCTCCAAGCGTGTCCGTGTCTCCTGGACA |
| | | GACCGCCAGAATCACATGTAGCGGCGGAGGCAGCTACACCGGCAGC |
| | | TACTACTATGGCTGGTATCAGCAGAAGCCCGGACAGGCCCCTGTGAC |
| | | CGTGATCTACTACAACAACAAGCGGCCCAGCGACATCCCCGAGAGAT |
| | | TTTCTGGCTCTCTGAGCGGCACCACCAACACACTGACAATCTCTGGC |
| | | GTGCAGGCCGAGGACGAGGCCGATTACTATTGTGGCAGCGCCGATAA |
| | | TAGCGGCGACGCCTTTGGCACCGGCACCAAAGTTACAGTGCTA |
| 34 | DNA | AGCGGCGGAGGCAGCTACACCGGCAGCTACTACTATGGC |
| 35 | DNA | TACAACAACAAGCGGCCCAGC |
| 36 | DNA | GGCAGCGCCGATAATAGCGGCGACGCC |
| 37 | PRT | EVQLLESGGGLVQPGGSLRLSCAASGFTFSSYPMGWVRQAPGKGLEWV |
| | | AGIDDDGDSDTRYAPAVKGRATISRDNSKNTVYLQMNSLRAEDTAVYY |
| | | CAKHTGIGANSAGSIDAWGQGTLVTVSSAKTTPPSVYPLAPGSAAQTNS |
| | | MVTLGCLVKGYFPEPVTVTWNSGSLSSGVHTFPAVLQSDLYTLSSSVTV |
| | | PSSTWPSETVTCNVAHPASSTKVDKKIVPRDCGCKPCICTVPEVSSVFIFP |
| | | PKPKDVLTITLTPKVTCVVVDISKDDPEVQFSWFVDDVEVHTAQTQPRE |
| | | EQFNSTFRSVSELPIMHQDWLNGKEFKCRVNSAAFPAPIEKTISKTKGRP |
| | | KAPQVYTIPPPKEQMAKDKVSLTCMITDFFPEDITVEWQWNGQPAENYK |
| | | NTQPIMDTDGSYFVYSKLNVQKSNWEAGNTFTCSVLHEGLHNHHTEKS |
| | | LSHSPGK |
| 38 | PRT | SYELTQPPSVSVSPGQTARITCSGGGSYTGSYYYGWYQQKPGQAPVTVI |
| | | YYNNKRPSDIPERFSGSLSGTTNTLTISGVQAEDEADYYCGSADNSGDAF |
| | | GTGTKVTVLGQPKSSPSVTLFPPSSEELETNKATLVCTITDFYPGVVTVD |
| | | WKVDGTPVTQGMETTQPSKQSNNKYMASSYLTLTARAWERHSSYSCQ |
| | | VTHEGHTVEKSLSRADCS |
| 39 | DNA | GAAGTTCAGCTGCTGGAATCTGGCGGCGGACTGGTTCAACCTGGCGG |
| | | ATCTCTGAGACTGAGCTGTGCCGCCAGCGGCTTCACCTTTAGCAGCT |
| | | ATCCTATGGGCTGGGTCCGACAGGCCCCTGGCAAAGGACTTGAATGG |
| | | GTGGCCGGCATCGACGACGATGGCGATAGCGATACAAGATACGCCC |
| | | CTGCCGTGAAGGGCAGAGCCACCATCTCCAGAGACAACAGCAAGAA |
| | | CACCGTGTACCTGCAGATGAACAGCCTGAGAGCCGAGGACACCGCC |
| | | GTGTACTATTGTGCCAAGCACACAGGCATCGGCGCCAATTCTGCCGG |
| | | CTCTATTGATGCCTGGGGCCAGGGAACACTGGTCACAGTTTCTTCAG |
| | | CCAAGACCACCCCCCCCAGCGTGTACCCTCTGGCTCCTGGATCTGCC |
| | | GCCCAGACCAACAGCATGGTCACCCTGGGCTGCCTCGTGAAGGGCTA |
| | | CTTCCCTGAGCCTGTGACCGTGACCTGGAACAGCGGCTCTCTGTCTAG |
| | | CGGCGTGCACACCTTTCCAGCCGTGCTGCAGAGCGACCTGTACACCC |
| | | TGAGCAGCAGCGTGACCGTGCCTAGCAGCACCTGGCCTAGCGAGACA |
| | | GTGACCTGCAACGTGGCCCACCCTGCCAGCAGCACAAAGGTGGACA |
| | | AGAAAATCGTGCCCCGGGACTGCGGCTGCAAGCCCTGTATCTGTACC |
| | | GTGCCCGAGGTGTCCAGCGTGTTCATCTTCCCACCCAAGCCCAAGGA |
| | | CGTGCTGACCATCACCCTGACCCCCAAAGTGACCTGTGTGGTGGTGG |
| | | ACATCAGCAAGGACGACCCCGAGGTGCAGTTCAGTTGGTTCGTGGAC |
| | | GACGTGGAAGTGCACACAGCCCAGACCCAGCCCAGAGAGGAACAGT |
| | | TCAACAGCACCTTCAGAAGCGTGTCCGAGCTGCCCATCATGCACCAG |
| | | GACTGGCTGAACGGCAAAGAGTTCAAGTGCAGAGTGAACAGCGCCG |
| | | CCTTCCCTGCCCCCATCGAGAAAACCATCTCCAAGACCAAGGGCAGA |
| | | CCCAAGGCCCCTCAGGTGTACACAATCCCCCCACCCAAAGAACAGAT |
| | | GGCCAAGGACAAGGTGTCCCTGACCTGCATGATCACCGATTTCTTCC |
| | | CAGAGGACATCACCGTGGAATGGCAGTGGAACGGCCAGCCCGCCGA |
| | | GAACTACAAGAACACCCAGCCTATCATGGACACCGACGGCAGCTACT |
| | | TCGTGTACAGCAAGCTGAACGTGCAGAAGTCCAACTGGGAGGCCGG |
| | | CAACACCTTCACCTGTAGCGTGCTGCACGAGGGCCTGCACAATCACC |
| | | ACACCGAGAAGTCCCTGTCCCACAGCCCTGGCAAG |
| 40 | DNA | AGCTATGAGCTGACACAGCCTCCAAGCGTGTCCGTGTCTCCTGGACA |
| | | GACCGCCAGAATCACATGTAGCGGCGGAGGCAGCTACACCGGCAGC |
| | | TACTACTATGGCTGGTATCAGCAGAAGCCCGGACAGGCCCCTGTGAC |
| | | CGTGATCTACTACAACAACAAGCGGCCCAGCGACATCCCCGAGAGAT |
| | | TTTCTGGCTCTCTGAGCGGCACCACCAACACACTGACAATCTCTGGC |
| | | GTGCAGGCCGAGGACGAGGCCGATTACTATTGTGGCAGCGCCGATAA |
| | | TAGCGGCGACGCCTTTGGCACCGGCACCAAAGTTACAGTGCTAGGCC |
| | | AGCCCAAGAGCAGCCCTAGCGTGACCCTGTTCCCTCCAAGCAGCGAG |
| | | GAACTGGAAACAAACAAGGCCACCCTCGTGTGCACCATCACCGACTT |
| | | CTACCCCGGCGTCGTGACCGTGGACTGGAAGGTGGACGGCACCCCAG |
| | | TGACCCAGGGCATGGAAACCACCCAGCCCAGCAAGCAGAGCAACAA |
| | | CAAGTACATGGCCAGCAGCTACCTGACCCTGACCGCCAGAGCCTGGG |
| | | AGAGACACAGCTCCTACAGCTGCCAAGTGACCCACGAGGGCCACAC |
| | | CGTGGAAAAGAGCCTGAGCAGAGCCGACTGCAGC |
| 41 | PRT | EVQLLESGGGLVQPGGSLRLSCAASGFTFSSYGMHWVRQAPGKGLEWV |
| | | SAIGTGGDTYYADSVMGRFTISRDNSKNTLYLQMNSLRAEDTAVYYCA |
| | | RRDDYTSRDAFDVWGQGTLVTVSS |
| 42 | PRT | SYGMH |
| 43 | PRT | AIGTGGDTYYADSVMG |
| 44 | PRT | RDDYTSRDAFDV |
| 45 | PRT | QSVLTQPPSASGTPGQRVTISCSGSSSNIGSNTVNWYQQLPGTAPKLLIYY |
| | | DDLLPSGVPDRFSGSKSGTSASLAISGLRSEDEADYYCAAWDDSLNGYV |
| | | VFGGGTKLTVL |
| 46 | PRT | SGSSSNIGSNTVN |
| 47 | PRT | YDDLLPS |
| 48 | PRT | AAWDDSLNGYVV |
| 49 | DNA | GAAGTTCAGCTGCTGGAATCTGGCGGCGGACTGGTTCAACCTGGCGG |
| | | ATCTCTGAGACTGAGCTGTGCCGCCAGCGGCTTCACCTTTAGCAGCT |
| | | ATGGCATGCACTGGGTCCGACAGGCCCCTGGCAAAGGACTTGAATGG |
| | | GTGTCCGCCATCGGCACAGGCGGCGATACCTACTATGCCGATAGCGT |
| | | GATGGGCAGATTCACCATCAGCCGGGACAACAGCAAGAACACCCTG |
| | | TACCTGCAGATGAACAGCCTGAGAGCCGAGGACACCGCCGTGTACTA |
| | | TTGCGCCAGAAGGGACGACTACACCAGCAGGGATGCCTTCGATGTGT |
| | | GGGGCCAGGGAACACTGGTTACCGTTTCTTCA |
| 50 | DNA | AGCTATGGCATGCAC |
| 51 | DNA | GCCATCGGCACAGGCGGCGATACCTACTATGCCGATAGCGTGATGGG |
| | | C |
| 52 | DNA | AGGGACGACTACACCAGCAGGGATGCCTTCGATGTG |
| 53 | DNA | CAGTCTGTTCTGACACAGCCTCCTAGCGCCTCTGGCACACCTGGACA |
| | | GAGAGTGACCATCAGCTGTAGCGGCAGCAGCTCCAACATCGGCAGC |
| | | AACACCGTGAACTGGTATCAGCAGCTGCCTGGCACAGCCCCTAAACT |
| | | GCTGATCTACTACGACGACCTGCTGCCTAGCGGCGTGCCCGATAGAT |
| | | TTTCTGGCAGCAAGAGCGGCACAAGCGCCAGCCTGGCTATCTCTGGA |
| | | CTGAGATCTGAGGACGAGGCCGACTACTATTGCGCCGCCTGGGACGA |
| | | TAGCCTGAACGGCTATGTGGTTTTCGGCGGAGGCACCAAGCTGACCG |
| | | TGCTA |
| 54 | DNA | AGCGGCAGCAGCTCCAACATCGGCAGCAACACCGTGAAC |
| 55 | DNA | TACGACGACCTGCTGCCTAGC |
| 56 | DNA | GCCGCCTGGGACGATAGCCTGAACGGCTATGTGGTT |
| 57 | PRT | EVQLLESGGGLVQPGGSLRLSCAASGFTFSSYGMHWVRQAPGKGLEWV |
| | | SAIGTGGDTYYADSVMGRFTISRDNSKNTLYLQMNSLRAEDTAVYYCA |
| | | RRDDYTSRDAFDVWGQGTLVTVSSASTKGPSVFPLAPSSKSTSGGTAAL |
| | | GCLVKDYFPEPVTVSWNSGALTSGVHTFPAVLQSSGLYSLSSVVTVPSSS |
| | | LGTQTYICNVNHKPSNTKVDKKVEPKSCDKTHTCPPCPAPELLGGPSVFL |
| | | FPPKPKDTLMISRTPEVTCVVVDVSHEDPEVKFNWYVDGVEVHNAKTK |
| | | PREEQYNSTYRVVSVLTVLHQDWLNGKEYKCKVSNKALPAPIEKTISKA |
| | | KGQPREPQVYTLPPSRDELTKNQVSLTCLVKGFYPSDIAVEWESNGQPE |
| | | NNYKTTPPVLDSDGSFFLYSKLTVDKSRWQQGNVFSCSVMHEALHNHY |
| | | TQKSLSLSPG |
| 58 | PRT | QSVLTQPPSASGTPGQRVTISCSGSSSNIGSNTVNWYQQLPGTAPKLLIYY |
| | | DDLLPSGVPDRFSGSKSGTSASLAISGLRSEDEADYYCAAWDDSLNGYV |
| | | VFGGGTKLTVLGQPKAAPSVTLFPPSSEELQANKATLVCLISDFYPGAVT |
| | | VAWKADSSPVKAGVETTTPSKQSNNKYAASSYLSLTPEQWKSHRSYSC |
| | | QVTHEGSTVEKTVAPTECS |
| 59 | DNA | GAAGTTCAGCTGCTGGAATCTGGCGGCGGACTGGTTCAACCTGGCGG |
| | | ATCTCTGAGACTGAGCTGTGCCGCCAGCGGCTTCACCTTTAGCAGCT |
| | | ATGGCATGCACTGGGTCCGACAGGCCCCTGGCAAAGGACTTGAATGG |
| | | GTGTCCGCCATCGGCACAGGCGGCGATACCTACTATGCCGATAGCGT |
| | | GATGGGCAGATTCACCATCAGCCGGGACAACAGCAAGAACACCCTG |
| | | TACCTGCAGATGAACAGCCTGAGAGCCGAGGACACCGCCGTGTACTA |
| | | TTGCGCCAGAAGGGACGACTACACCAGCAGGGATGCCTTCGATGTGT |
| | | GGGGCCAGGGAACACTGGTTACCGTTTCTTCAGCCAGCACCAAGGGC |
| | | CCCAGCGTGTTCCCTCTGGCCCCTAGCAGCAAGAGCACATCTGGCGG |
| | | AACAGCCGCCCTGGGCTGCCTCGTGAAGGACTACTTTCCCGAGCCCG |
| | | TGACCGTGTCCTGGAACTCTGGCGCTCTGACAAGCGGCGTGCACACC |
| | | TTTCCAGCCGTGCTGCAGAGCAGCGGCCTGTACTCTCTGAGCAGCGT |
| | | CGTGACAGTGCCCAGCAGCTCTCTGGGCACCCAGACCTACATCTGCA |
| | | ACGTGAACCACAAGCCCAGCAACACCAAGGTGGACAAGAAGGTGGA |
| | | ACCCAAGAGCTGCGACAAGACCCACACCTGTCCCCCTTGTCCTGCCC |
| | | CCGAACTGCTGGGAGGCCCTTCCGTGTTCCTGTTCCCCCCAAAGCCCA |
| | | AGGACACCCTGATGATCAGCCGGACCCCCGAAGTGACCTGCGTGGTG |
| | | GTGGATGTGTCCCACGAGGACCCTGAAGTGAAGTTCAATTGGTACGT |
| | | GGACGGCGTGGAAGTGCACAACGCCAAGACCAAGCCTAGAGAGGAA |
| | | CAGTACAACAGCACCTACCGGGTGGTGTCCGTGCTGACAGTGCTGCA |
| | | CCAGGACTGGCTGAACGGCAAAGAGTACAAGTGCAAGGTGTCCAAC |
| | | AAGGCCCTGCCTGCCCCCATCGAGAAAACCATCAGCAAGGCCAAGG |
| | | GCCAGCCCCGCGAACCCCAGGTGTACACACTGCCCCCAAGCAGGGAC |
| | | GAGCTGACCAAGAACCAGGTGTCCCTGACCTGTCTCGTGAAAGGCTT |
| | | CTACCCCTCCGATATCGCCGTGGAATGGGAGAGCAACGGCCAGCCCG |
| | | AGAACAACTACAAGACCACCCCCCCTGTGCTGGACAGCGACGGCTCA |
| | | TTCTTCCTGTACAGCAAGCTGACCGTGGACAAGTCCCGGTGGCAGCA |
| | | GGGCAACGTGTTCAGCTGCAGCGTGATGCACGAGGCCCTGCACAACC |
| | | ACTACACCCAGAAGTCCCTGAGCCTGAGCCCTGGC |
| 60 | DNA | CAGTCTGTTCTGACACAGCCTCCTAGCGCCTCTGGCACACCTGGACA |
| | | GAGAGTGACCATCAGCTGTAGCGGCAGCAGCTCCAACATCGGCAGC |
| | | AACACCGTGAACTGGTATCAGCAGCTGCCTGGCACAGCCCCTAAACT |
| | | GCTGATCTACTACGACGACCTGCTGCCTAGCGGCGTGCCCGATAGAT |
| | | TTTCTGGCAGCAAGAGCGGCACAAGCGCCAGCCTGGCTATCTCTGGA |
| | | CTGAGATCTGAGGACGAGGCCGACTACTATTGCGCCGCCTGGGACGA |
| | | TAGCCTGAACGGCTATGTGGTTTTCGGCGGAGGCACCAAGCTGACCG |
| | | TGCTAGGCCAGCCTAAAGCCGCCCCTAGCGTGACCCTGTTCCCTCCA |
| | | AGCAGCGAGGAACTGCAGGCCAACAAGGCCACCCTCGTGTGCCTGAT |
| | | CAGCGACTTCTATCCTGGCGCCGTGACCGTGGCCTGGAAGGCCGATA |
| | | GCTCTCCTGTGAAGGCCGGCGTGGAAACCACCACCCCTAGCAAGCAG |
| | | AGCAACAACAAATACGCCGCCAGCAGCTACCTGAGCCTGACCCCCGA |
| | | GCAGTGGAAGTCCCACAGATCCTACAGCTGCCAAGTGACCCACGAGG |
| | | GCAGCACCGTGGAAAAGACAGTGGCCCCTACCGAGTGCAGC |
| 61 | PRT | EVQLLESGGGLVQPGGSLRLSCAASGFTFSSYEMNWVRQAPGKGLEWV |
| | | SGISWNSGSIGYADSVKGRFTISRDNSKNTLYLQMNSLRAEDTAVYYCA |
| | | RSGYSSSWFDPDFDYWGQGTLVTVSS |
| 62 | PRT | SYEMN |
| 63 | PRT | GISWNSGSIGYADSVKG |
| 64 | PRT | SGYSSSWFDPDFDY |
| 65 | PRT | QSVLTQPPSASGTPGQRVTISCTGSSSNIGAGYDVHWYQQLPGTAPKLLI |
| | | YGNSNRPSGVPDRFSGSKSGTSASLAISGLRSEDEADYYCSSYAGSNPYV |
| | | VFGGGTKLTVL |
| 66 | PRT | TGSSSNIGAGYDVH |
| 67 | PRT | GNSNRPS |
| 68 | PRT | SSYAGSNPYVV |
| 69 | DNA | GAAGTTCAGCTGCTGGAATCTGGCGGCGGACTGGTTCAACCTGGCGG |
| | | ATCTCTGAGACTGAGCTGTGCCGCCAGCGGCTTCACCTTTAGCAGCT |
| | | ACGAGATGAACTGGGTCCGACAGGCCCCTGGCAAAGGCCTTGAATG |
| | | GGTGTCCGGCATCAGCTGGAATAGCGGCTCTATCGGCTACGCCGACA |
| | | GCGTGAAGGGCAGATTCACCATCAGCCGGGACAACAGCAAGAACAC |
| | | CCTGTACCTGCAGATGAACAGCCTGAGAGCCGAGGACACCGCCGTGT |
| | | ACTACTGTGCCAGAAGCGGCTACAGCAGCTCTTGGTTTGACCCCGAC |
| | | TTCGACTATTGGGGCCAGGGCACACTGGTCACAGTCTCTTCA |
| 70 | DNA | AGCTACGAGATGAAC |
| 71 | DNA | GGCATCAGCTGGAATAGCGGCTCTATCGGCTACGCCGACAGCGTGAA |
| | | GGGC |
| 72 | DNA | AGCGGCTACAGCAGCTCTTGGTTTGACCCCGACTTCGACTAT |
| 73 | DNA | CAGTCTGTTCTGACACAGCCTCCTAGCGCCTCTGGCACACCTGGACA |
| | | GAGAGTGACCATCAGCTGTACCGGCAGCAGCTCCAATATCGGAGCCG |
| | | GCTATGACGTGCACTGGTATCAGCAGCTGCCTGGCACAGCCCCTAAA |
| | | CTGCTGATCTACGGCAACAGCAACAGACCCAGCGGCGTGCCCGATAG |
| | | ATTTTCCGGCTCTAAGAGCGGCACAAGCGCCAGCCTGGCTATCTCTG |
| | | GACTGAGATCTGAGGACGAGGCCGACTACTACTGCAGCAGCTATGCC |
| | | GGCAGCAACCCCTACGTTGTGTTTGGCGGAGGCACCAAGCTGACCGT |
| | | TCTA |
| 74 | DNA | ACCGGCAGCAGCTCCAATATCGGAGCCGGCTATGACGTGCAC |
| 75 | DNA | GGCAACAGCAACAGACCCAGC |
| 76 | DNA | AGCAGCTATGCCGGCAGCAACCCCTACGTTGTG |
| 77 | PRT | EVQLLESGGGLVQPGGSLRLSCAASGFTFSSYEMNWVRQAPGKGLEWV |
| | | SGISWNSGSIGYADSVKGRFTISRDNSKNTLYLQMNSLRAEDTAVYYCA |
| | | RSGYSSSWFDPDFDYWGQGTLVTVSSASTKGPSVFPLAPSSKSTSGGTA |
| | | ALGCLVKDYFPEPVTVSWNSGALTSGVHTFPAVLQSSGLYSLSSVVTVP |
| | | SSSLGTQTYICNVNHKPSNTKVDKKVEPKSCDKTHTCPPCPAPELLGGPS |
| | | VFLFPPKPKDTLMISRTPEVTCVVVDVSHEDPEVKFNWYVDGVEVHNA |
| | | KTKPREEQYNSTYRVVSVLTVLHQDWLNGKEYKCKVSNKALPAPIEKTI |
| | | SKAKGQPREPQVYTLPPSRDELTKNQVSLTCLVKGFYPSDIAVEWESNG |
| | | QPENNYKTTPPVLDSDGSFFLYSKLTVDKSRWQQGNVFSCSVMHEALH |
| | | NHYTQKSLSLSPG |
| 78 | PRT | QSVLTQPPSASGTPGQRVTISCTGSSSNIGAGYDVHWYQQLPGTAPKLLI |
| | | YGNSNRPSGVPDRFSGSKSGTSASLAISGLRSEDEADYYCSSYAGSNPYV |
| | | VFGGGTKLTVLGQPKAAPSVTLFPPSSEELQANKATLVCLISDFYPGAVT |
| | | VAWKADSSPVKAGVETTTPSKQSNNKYAASSYLSLTPEQWKSHRSYSC |
| | | QVTHEGSTVEKTVAPTECS |
| 79 | DNA | GAAGTTCAGCTGCTGGAATCTGGCGGCGGACTGGTTCAACCTGGCGG |
| | | ATCTCTGAGACTGAGCTGTGCCGCCAGCGGCTTCACCTTTAGCAGCT |
| | | ACGAGATGAACTGGGTCCGACAGGCCCCTGGCAAAGGCCTTGAATG |
| | | GGTGTCCGGCATCAGCTGGAATAGCGGCTCTATCGGCTACGCCGACA |
| | | GCGTGAAGGGCAGATTCACCATCAGCCGGGACAACAGCAAGAACAC |
| | | CCTGTACCTGCAGATGAACAGCCTGAGAGCCGAGGACACCGCCGTGT |
| | | ACTACTGTGCCAGAAGCGGCTACAGCAGCTCTTGGTTTGACCCCGAC |
| | | TTCGACTATTGGGGCCAGGGCACACTGGTCACAGTCTCTTCAGCCAG |
| | | CACCAAGGGCCCCAGCGTGTTCCCTCTGGCCCCTAGCAGCAAGAGCA |
| | | CATCTGGCGGAACAGCCGCCCTGGGCTGCCTCGTGAAGGACTACTTT |
| | | CCCGAGCCCGTGACCGTGTCCTGGAACTCTGGCGCTCTGACAAGCGG |
| | | CGTGCACACCTTTCCAGCCGTGCTGCAGAGCAGCGGCCTGTACTCTCT |
| | | GAGCAGCGTCGTGACAGTGCCCAGCAGCTCTCTGGGCACCCAGACCT |
| | | ACATCTGCAACGTGAACCACAAGCCCAGCAACACCAAGGTGGACAA |
| | | GAAGGTGGAACCCAAGAGCTGCGACAAGACCCACACCTGTCCCCCTT |
| | | GTCCTGCCCCCGAACTGCTGGGAGGCCCTTCCGTGTTCCTGTTCCCCC |
| | | CAAAGCCCAAGGACACCCTGATGATCAGCCGGACCCCCGAAGTGAC |
| | | CTGCGTGGTGGTGGATGTGTCCCACGAGGACCCTGAAGTGAAGTTCA |
| | | ATTGGTACGTGGACGGCGTGGAAGTGCACAACGCCAAGACCAAGCC |
| | | TAGAGAGGAACAGTACAACAGCACCTACCGGGTGGTGTCCGTGCTGA |
| | | CAGTGCTGCACCAGGACTGGCTGAACGGCAAAGAGTACAAGTGCAA |
| | | GGTGTCCAACAAGGCCCTGCCTGCCCCCATCGAGAAAACCATCAGCA |
| | | AGGCCAAGGGCCAGCCCCGCGAACCCCAGGTGTACACACTGCCCCCA |
| | | AGCAGGGACGAGCTGACCAAGAACCAGGTGTCCCTGACCTGTCTCGT |
| | | GAAAGGCTTCTACCCCTCCGATATCGCCGTGGAATGGGAGAGCAACG |
| | | GCCAGCCCGAGAACAACTACAAGACCACCCCCCCTGTGCTGGACAGC |
| | | GACGGCTCATTCTTCCTGTACAGCAAGCTGACCGTGGACAAGTCCCG |
| | | GTGGCAGCAGGGCAACGTGTTCAGCTGCAGCGTGATGCACGAGGCCC |
| | | TGCACAACCACTACACCCAGAAGTCCCTGAGCCTGAGCCCTGGC |
| 80 | DNA | CAGTCTGTTCTGACACAGCCTCCTAGCGCCTCTGGCACACCTGGACA |
| | | GAGAGTGACCATCAGCTGTACCGGCAGCAGCTCCAATATCGGAGCCG |
| | | GCTATGACGTGCACTGGTATCAGCAGCTGCCTGGCACAGCCCCTAAA |
| | | CTGCTGATCTACGGCAACAGCAACAGACCCAGCGGCGTGCCCGATAG |
| | | ATTTTCCGGCTCTAAGAGCGGCACAAGCGCCAGCCTGGCTATCTCTG |
| | | GACTGAGATCTGAGGACGAGGCCGACTACTACTGCAGCAGCTATGCC |
| | | GGCAGCAACCCCTACGTTGTGTTTGGCGGAGGCACCAAGCTGACCGT |
| | | TCTAGGCCAGCCTAAAGCCGCCCCTAGCGTGACCCTGTTCCCTCCAA |
| | | GCAGCGAGGAACTGCAGGCCAACAAGGCCACCCTCGTGTGCCTGATC |
| | | AGCGACTTCTATCCTGGCGCCGTGACCGTGGCCTGGAAGGCCGATAG |
| | | CTCTCCTGTGAAGGCCGGCGTGGAAACCACCACCCCTAGCAAGCAGA |
| | | GCAACAACAAATACGCCGCCAGCAGCTACCTGAGCCTGACCCCCGAG |
| | | CAGTGGAAGTCCCACAGATCCTACAGCTGCCAAGTGACCCACGAGGG |
| | | CAGCACCGTGGAAAAGACAGTGGCCCCTACCGAGTGCAGC |
| 81 | PRT | EVQLLESGGGLVQTGGSLRLSCAASGFTFSDYAMSWVRQAPGKGLEWV |
| | | SWIYYDSGSKYYADSVKGRFTISRDNSKNTLYLQMNSLRAEDTAVYYC |
| | | AKLNGDFDYWGQGTLVTVSS |
| 82 | PRT | DYAMS |
| 83 | PRT | WIYYDSGSKYYADSVKG |
| 84 | PRT | LNGDFDY |
| 85 | PRT | QSVLTQPPSASGTPGQRVTISCSGSSSNIGNNDVSWYQQLPGTAPKLLIY |
| | | ADSHRPSGVPDRFSGSKSGTSASLAISGLRSEDEADYYCGAWDSSLSGY |
| | | VFGGGTKLTVL |
| 86 | PRT | SGSSSNIGNNDVS |
| 87 | PRT | ADSHRPS |
| 88 | PRT | GAWDSSLSGYV |
| 89 | DNA | GAAGTTCAGCTGCTGGAATCTGGCGGCGGACTGGTTCAAACAGGCGG |
| | | CTCTCTGAGACTGAGCTGTGCCGCCTCTGGCTTCACCTTCAGCGATTA |
| | | CGCCATGAGCTGGGTCCGACAGGCCCCTGGAAAAGGCCTTGAATGGG |
| | | TGTCCTGGATCTACTACGACAGCGGCAGCAAGTACTACGCCGACAGC |
| | | GTGAAGGGCAGATTCACCATCAGCCGGGACAACAGCAAGAACACCC |
| | | TGTACCTGCAGATGAACAGCCTGAGAGCCGAGGACACCGCCGTGTAC |
| | | TATTGCGCCAAGCTGAACGGCGACTTCGACTATTGGGGCCAGGGCAC |
| | | ACTGGTCACAGTCTCTTCA |
| 90 | DNA | GATTACGCCATGAGC |
| 91 | DNA | TGGATCTACTACGACAGCGGCAGCAAGTACTACGCCGACAGCGTGAA |
| | | GGGC |
| 92 | DNA | CTGAACGGCGACTTCGACTAT |
| 93 | DNA | CAGTCTGTTCTGACACAGCCTCCTAGCGCCTCTGGCACACCTGGACA |
| | | GAGAGTGACCATCAGCTGTAGCGGCAGCAGCTCCAACATCGGCAAC |
| | | AACGACGTGTCCTGGTATCAGCAGCTGCCTGGCACAGCCCCTAAACT |
| | | GCTGATCTACGCCGACAGCCACAGACCTAGCGGCGTGCCAGATAGAT |
| | | TCAGCGGCTCTAAGAGCGGCACATCTGCCAGCCTGGCCATCTCTGGA |
| | | CTGAGATCTGAGGACGAGGCCGACTACTATTGCGGCGCCTGGGATTC |
| | | TAGCCTGAGCGGCTATGTTTTTGGCGGAGGCACCAAGCTGACCGTGC |
| | | TA |
| 94 | DNA | AGCGGCAGCAGCTCCAACATCGGCAACAACGACGTGTCC |
| 95 | DNA | GCCGACAGCCACAGACCTAGC |
| 96 | DNA | GGCGCCTGGGATTCTAGCCTGAGCGGCTATGTT |
| 97 | PRT | EVQLLESGGGLVQTGGSLRLSCAASGFTFSDYAMSWVRQAPGKGLEWV |
| | | SWIYYDSGSKYYADSVKGRFTISRDNSKNTLYLQMNSLRAEDTAVYYC |
| | | AKLNGDFDYWGQGTLVTVSSASTKGPSVFPLAPSSKSTSGGTAALGCLV |
| | | KDYFPEPVTVSWNSGALTSGVHTFPAVLQSSGLYSLSSVVTVPSSSLGTQ |
| | | TYICNVNHKPSNTKVDKKVEPKSCDKTHTCPPCPAPELLGGPSVFLFPPK |
| | | PKDTLMISRTPEVTCVVVDVSHEDPEVKFNWYVDGVEVHNAKTKPREE |
| | | QYNSTYRVVSVLTVLHQDWLNGKEYKCKVSNKALPAPIEKTISKAKGQ |
| | | PREPQVYTLPPSRDELTKNQVSLTCLVKGFYPSDIAVEWESNGQPENNY |
| | | KTTPPVLDSDGSFFLYSKLTVDKSRWQQGNVFSCSVMHEALHNHYTQK |
| | | SLSLSPG |
| 98 | PRT | QSVLTQPPSASGTPGQRVTISCSGSSSNIGNNDVSWYQQLPGTAPKLLIY |
| | | ADSHRPSGVPDRFSGSKSGTSASLAISGLRSEDEADYYCGAWDSSLSGY |
| | | VFGGGTKLTVLGQPKAAPSVTLFPPSSEELQANKATLVCLISDFYPGAVT |
| | | VAWKADSSPVKAGVETTTPSKQSNNKYAASSYLSLTPEQWKSHRSYSC |
| | | QVTHEGSTVEKTVAPTECS |
| 99 | DNA | GAAGTTCAGCTGCTGGAATCTGGCGGCGGACTGGTTCAAACAGGCGG |
| | | CTCTCTGAGACTGAGCTGTGCCGCCTCTGGCTTCACCTTCAGCGATTA |
| | | CGCCATGAGCTGGGTCCGACAGGCCCCTGGAAAAGGCCTTGAATGGG |
| | | TGTCCTGGATCTACTACGACAGCGGCAGCAAGTACTACGCCGACAGC |
| | | GTGAAGGGCAGATTCACCATCAGCCGGGACAACAGCAAGAACACCC |
| | | TGTACCTGCAGATGAACAGCCTGAGAGCCGAGGACACCGCCGTGTAC |
| | | TATTGCGCCAAGCTGAACGGCGACTTCGACTATTGGGGCCAGGGCAC |
| | | ACTGGTCACAGTCTCTTCAGCCAGCACCAAGGGCCCCAGCGTGTTCC |
| | | CTCTGGCCCCTAGCAGCAAGAGCACATCTGGCGGAACAGCCGCCCTG |
| | | GGCTGCCTCGTGAAGGACTACTTTCCCGAGCCCGTGACCGTGTCCTG |
| | | GAACTCTGGCGCTCTGACAAGCGGCGTGCACACCTTTCCAGCCGTGC |
| | | TGCAGAGCAGCGGCCTGTACTCTCTGAGCAGCGTCGTGACAGTGCCC |
| | | AGCAGCTCTCTGGGCACCCAGACCTACATCTGCAACGTGAACCACAA |
| | | GCCCAGCAACACCAAGGTGGACAAGAAGGTGGAACCCAAGAGCTGC |
| | | GACAAGACCCACACCTGTCCCCCTTGTCCTGCCCCCGAACTGCTGGG |
| | | AGGCCCTTCCGTGTTCCTGTTCCCCCCAAAGCCCAAGGACACCCTGAT |
| | | GATCAGCCGGACCCCCGAAGTGACCTGCGTGGTGGTGGATGTGTCCC |
| | | ACGAGGACCCTGAAGTGAAGTTCAATTGGTACGTGGACGGCGTGGA |
| | | AGTGCACAACGCCAAGACCAAGCCTAGAGAGGAACAGTACAACAGC |
| | | ACCTACCGGGTGGTGTCCGTGCTGACAGTGCTGCACCAGGACTGGCT |
| | | GAACGGCAAAGAGTACAAGTGCAAGGTGTCCAACAAGGCCCTGCCT |
| | | GCCCCCATCGAGAAAACCATCAGCAAGGCCAAGGGCCAGCCCCGCG |
| | | AACCCCAGGTGTACACACTGCCCCCAAGCAGGGACGAGCTGACCAA |
| | | GAACCAGGTGTCCCTGACCTGTCTCGTGAAAGGCTTCTACCCCTCCG |
| | | ATATCGCCGTGGAATGGGAGAGCAACGGCCAGCCCGAGAACAACTA |
| | | CAAGACCACCCCCCCTGTGCTGGACAGCGACGGCTCATTCTTCCTGT |
| | | ACAGCAAGCTGACCGTGGACAAGTCCCGGTGGCAGCAGGGCAACGT |
| | | GTTCAGCTGCAGCGTGATGCACGAGGCCCTGCACAACCACTACACCC |
| | | AGAAGTCCCTGAGCCTGAGCCCTGGC |
| 100 | DNA | CAGTCTGTTCTGACACAGCCTCCTAGCGCCTCTGGCACACCTGGACA |
| | | GAGAGTGACCATCAGCTGTAGCGGCAGCAGCTCCAACATCGGCAAC |
| | | AACGACGTGTCCTGGTATCAGCAGCTGCCTGGCACAGCCCCTAAACT |
| | | GCTGATCTACGCCGACAGCCACAGACCTAGCGGCGTGCCAGATAGAT |
| | | TCAGCGGCTCTAAGAGCGGCACATCTGCCAGCCTGGCCATCTCTGGA |
| | | CTGAGATCTGAGGACGAGGCCGACTACTATTGCGGCGCCTGGGATTC |
| | | TAGCCTGAGCGGCTATGTTTTTGGCGGAGGCACCAAGCTGACCGTGC |
| | | TAGGCCAGCCTAAAGCCGCCCCTAGCGTGACCCTGTTCCCTCCAAGC |
| | | AGCGAGGAACTGCAGGCCAACAAGGCCACCCTCGTGTGCCTGATCAG |
| | | CGACTTCTATCCTGGCGCCGTGACCGTGGCCTGGAAGGCCGATAGCT |
| | | CTCCTGTGAAGGCCGGCGTGGAAACCACCACCCCTAGCAAGCAGAGC |
| | | AACAACAAATACGCCGCCAGCAGCTACCTGAGCCTGACCCCCGAGCA |
| | | GTGGAAGTCCCACAGATCCTACAGCTGCCAAGTGACCCACGAGGGCA |
| | | GCACCGTGGAAAAGACAGTGGCCCCTACCGAGTGCAGC |
| 101 | PRT | EVQLLESGGGLVQPGGSLRLSCAASGFTFSSYEMNWVRQAPGKGLEWV |
| | | SGISWNSGSIGYADSVKGRFTISRDNSKNTLYLQMNSLRAEDTAVYYCA |
| | | RSGYSSSWFDPDFDYWGQGTLVTVSS |
| 102 | PRT | SYEMN |
| 103 | PRT | GISWNSGSIGYADSVKG |
| 104 | PRT | SGYSSSWFDPDFDY |
| 105 | PRT | QSVLTQPPSVSGAPGQRVTISCTGSSSNIGAGYDVHWYQQLPGTAPKLLI |
| | | YGNSNRPSGVPDRFSGSKSGTSASLAITGLQAEDEADYYCSSYAGSNPY |
| | | VVFGGGTKLTVL |
| 106 | PRT | TGSSSNIGAGYDVH |
| 107 | PRT | GNSNRPS |
| 108 | PRT | SSYAGSNPYVV |
| 109 | DNA | GAAGTTCAGCTGCTGGAATCTGGCGGCGGACTGGTTCAACCTGGCGG |
| | | ATCTCTGAGACTGAGCTGTGCCGCCAGCGGCTTCACCTTTAGCAGCT |
| | | ACGAGATGAACTGGGTCCGACAGGCCCCTGGCAAAGGCCTTGAATG |
| | | GGTGTCCGGCATCAGCTGGAATAGCGGCTCTATCGGCTACGCCGACA |
| | | GCGTGAAGGGCAGATTCACCATCAGCCGGGACAACAGCAAGAACAC |
| | | CCTGTACCTGCAGATGAACAGCCTGAGAGCCGAGGACACCGCCGTGT |
| | | ACTACTGTGCCAGAAGCGGCTACAGCAGCTCTTGGTTTGACCCCGAC |
| | | TTCGACTATTGGGGCCAGGGCACACTGGTCACAGTCTCTTCA |
| 110 | DNA | AGCTACGAGATGAAC |
| 111 | DNA | GGCATCAGCTGGAATAGCGGCTCTATCGGCTACGCCGACAGCGTGAA |
| | | GGGC |
| 112 | DNA | AGCGGCTACAGCAGCTCTTGGTTTGACCCCGACTTCGACTAT |
| 113 | DNA | CAGTCTGTTCTGACACAGCCTCCATCTGTGTCTGGCGCCCCTGGACAG |
| | | AGAGTGACCATCAGCTGTACAGGCAGCAGCTCCAATATCGGAGCCGG |
| | | CTATGACGTGCACTGGTATCAGCAGCTGCCTGGCACAGCCCCTAAAC |
| | | TGCTGATCTACGGCAACAGCAACAGACCCAGCGGCGTGCCCGATAGA |
| | | TTTTCCGGCTCTAAGAGCGGCACAAGCGCCAGCCTGGCTATTACTGG |
| | | ACTGCAGGCCGAGGACGAGGCCGACTACTACTGTTCTAGCTACGCCG |
| | | GCAGCAACCCCTACGTGGTGTTTGGCGGAGGCACCAAGCTGACAGTT |
| | | CTA |
| 114 | DNA | ACAGGCAGCAGCTCCAATATCGGAGCCGGCTATGACGTGCAC |
| 115 | DNA | GGCAACAGCAACAGACCCAGC |
| 116 | DNA | TCTAGCTACGCCGGCAGCAACCCCTACGTGGTG |
| 117 | PRT | EVQLLESGGGLVQPGGSLRLSCAASGFTFSSYEMNWVRQAPGKGLEWV |
| | | SGISWNSGSIGYADSVKGRFTISRDNSKNTLYLQMNSLRAEDTAVYYCA |
| | | RSGYSSSWFDPDFDYWGQGTLVTVSSASTKGPSVFPLAPSSKSTSGGTA |
| | | ALGCLVKDYFPEPVTVSWNSGALTSGVHTFPAVLQSSGLYSLSSVVTVP |
| | | SSSLGTQTYICNVNHKPSNTKVDKKVEPKSCDKTHTCPPCPAPELLGGPS |
| | | VFLFPPKPKDTLMISRTPEVTCVVVDVSHEDPEVKFNWYVDGVEVHNA |
| | | KTKPREEQYNSTYRVVSVLTVLHQDWLNGKEYKCKVSNKALPAPIEKTI |
| | | SKAKGQPREPQVYTLPPSRDELTKNQVSLTCLVKGFYPSDIAVEWESNG |
| | | QPENNYKTTPPVLDSDGSFFLYSKLTVDKSRWQQGNVFSCSVMHEALH |
| | | NHYTQKSLSLSPG |
| 118 | PRT | QSVLTQPPSVSGAPGQRVTISCTGSSSNIGAGYDVHWYQQLPGTAPKLLI |
| | | YGNSNRPSGVPDRFSGSKSGTSASLAITGLQAEDEADYYCSSYAGSNPY |
| | | VVFGGGTKLTVLGQPKAAPSVTLFPPSSEELQANKATLVCLISDFYPGAV |
| | | TVAWKADSSPVKAGVETTTPSKQSNNKYAASSYLSLTPEQWKSHRSYS |
| | | CQVTHEGSTVEKTVAPTECS |
| 119 | DNA | GAAGTTCAGCTGCTGGAATCTGGCGGCGGACTGGTTCAACCTGGCGG |
| | | ATCTCTGAGACTGAGCTGTGCCGCCAGCGGCTTCACCTTTAGCAGCT |
| | | ACGAGATGAACTGGGTCCGACAGGCCCCTGGCAAAGGCCTTGAATG |
| | | GGTGTCCGGCATCAGCTGGAATAGCGGCTCTATCGGCTACGCCGACA |
| | | GCGTGAAGGGCAGATTCACCATCAGCCGGGACAACAGCAAGAACAC |
| | | CCTGTACCTGCAGATGAACAGCCTGAGAGCCGAGGACACCGCCGTGT |
| | | ACTACTGTGCCAGAAGCGGCTACAGCAGCTCTTGGTTTGACCCCGAC |
| | | TTCGACTATTGGGGCCAGGGCACACTGGTCACAGTCTCTTCAGCCAG |
| | | CACCAAGGGCCCCAGCGTGTTCCCTCTGGCCCCTAGCAGCAAGAGCA |
| | | CATCTGGCGGAACAGCCGCCCTGGGCTGCCTCGTGAAGGACTACTTT |
| | | CCCGAGCCCGTGACCGTGTCCTGGAACTCTGGCGCTCTGACAAGCGG |
| | | CGTGCACACCTTTCCAGCCGTGCTGCAGAGCAGCGGCCTGTACTCTCT |
| | | GAGCAGCGTCGTGACAGTGCCCAGCAGCTCTCTGGGCACCCAGACCT |
| | | ACATCTGCAACGTGAACCACAAGCCCAGCAACACCAAGGTGGACAA |
| | | GAAGGTGGAACCCAAGAGCTGCGACAAGACCCACACCTGTCCCCCTT |
| | | GTCCTGCCCCCGAACTGCTGGGAGGCCCTTCCGTGTTCCTGTTCCCCC |
| | | CAAAGCCCAAGGACACCCTGATGATCAGCCGGACCCCCGAAGTGAC |
| | | CTGCGTGGTGGTGGATGTGTCCCACGAGGACCCTGAAGTGAAGTTCA |
| | | ATTGGTACGTGGACGGCGTGGAAGTGCACAACGCCAAGACCAAGCC |
| | | TAGAGAGGAACAGTACAACAGCACCTACCGGGTGGTGTCCGTGCTGA |
| | | CAGTGCTGCACCAGGACTGGCTGAACGGCAAAGAGTACAAGTGCAA |
| | | GGTGTCCAACAAGGCCCTGCCTGCCCCCATCGAGAAAACCATCAGCA |
| | | AGGCCAAGGGCCAGCCCCGCGAACCCCAGGTGTACACACTGCCCCCA |
| | | AGCAGGGACGAGCTGACCAAGAACCAGGTGTCCCTGACCTGTCTCGT |
| | | GAAAGGCTTCTACCCCTCCGATATCGCCGTGGAATGGGAGAGCAACG |
| | | GCCAGCCCGAGAACAACTACAAGACCACCCCCCCTGTGCTGGACAGC |
| | | GACGGCTCATTCTTCCTGTACAGCAAGCTGACCGTGGACAAGTCCCG |
| | | GTGGCAGCAGGGCAACGTGTTCAGCTGCAGCGTGATGCACGAGGCCC |
| | | TGCACAACCACTACACCCAGAAGTCCCTGAGCCTGAGCCCTGGC |
| 120 | DNA | CAGTCTGTTCTGACACAGCCTCCATCTGTGTCTGGCGCCCCTGGACAG |
| | | AGAGTGACCATCAGCTGTACAGGCAGCAGCTCCAATATCGGAGCCGG |
| | | CTATGACGTGCACTGGTATCAGCAGCTGCCTGGCACAGCCCCTAAAC |
| | | TGCTGATCTACGGCAACAGCAACAGACCCAGCGGCGTGCCCGATAGA |
| | | TTTTCCGGCTCTAAGAGCGGCACAAGCGCCAGCCTGGCTATTACTGG |
| | | ACTGCAGGCCGAGGACGAGGCCGACTACTACTGTTCTAGCTACGCCG |
| | | GCAGCAACCCCTACGTGGTGTTTGGCGGAGGCACCAAGCTGACAGTT |
| | | CTAGGCCAGCCTAAAGCCGCCCCTAGCGTGACCCTGTTCCCTCCAAG |
| | | CAGCGAGGAACTGCAGGCCAACAAGGCCACCCTCGTGTGCCTGATCA |
| | | GCGACTTCTATCCTGGCGCCGTGACCGTGGCCTGGAAGGCCGATAGC |
| | | TCTCCTGTGAAGGCCGGCGTGGAAACCACCACCCCTAGCAAGCAGAG |
| | | CAACAACAAATACGCCGCCAGCAGCTACCTGAGCCTGACCCCCGAGC |
| | | AGTGGAAGTCCCACAGATCCTACAGCTGCCAAGTGACCCACGAGGGC |
| | | AGCACCGTGGAAAAGACAGTGGCCCCTACCGAGTGCAGC |
| 121 | PRT | EVQLLESGGGLVQPGGSLRLSCAASGFTFSSYAMSWVRQAPGKGLEWV |
| | | SAIGTGGDTYYADSVKGRFTISRDNSKNTLYLQMNSLRAEDTAVYYCA |
| | | RRDDYTSRDAFDYWGQGTLVTVSS |
| 122 | PRT | SYAMS |
| 123 | PRT | AIGTGGDTYYADSVKG |
| 124 | PRT | RDDYTSRDAFDY |
| 125 | PRT | QSVLTQPPSASGTPGQRVTISCSGSSSNIGSNTVNWYQQLPGTAPKLLIYY |
| | | DDLRPSGVPDRFSGSKSGTSASLAISGLQSEDEADYYCAAWDDSLNGYV |
| | | VFGGGTKLTVL |
| 126 | PRT | SGSSSNIGSNTVN |
| 127 | PRT | YDDLRPS |
| 128 | PRT | AAWDDSLNGYVV |
| 129 | DNA | GAAGTTCAGCTGCTGGAATCTGGCGGCGGACTGGTTCAACCTGGCGG |
| | | ATCTCTGAGACTGAGCTGTGCCGCCAGCGGCTTCACCTTTAGCAGCT |
| | | ACGCCATGAGCTGGGTCCGACAGGCTCCTGGCAAAGGCCTTGAATGG |
| | | GTGTCCGCCATTGGCACAGGCGGCGATACCTACTACGCCGACTCTGT |
| | | GAAGGGCAGATTCACCATCAGCCGGGACAACAGCAAGAACACCCTG |
| | | TACCTGCAGATGAACAGCCTGAGAGCCGAGGACACCGCCGTGTACTA |
| | | TTGCGCCAGAAGGGACGACTACACCAGCAGGGACGCCTTCGATTATT |
| | | GGGGCCAGGGCACACTGGTCACCGTTTCTTCA |
| 130 | DNA | AGCTACGCCATGAGC |
| 131 | DNA | GCCATTGGCACAGGCGGCGATACCTACTACGCCGACTCTGTGAAGGG |
| | | C |
| 132 | DNA | AGGGACGACTACACCAGCAGGGACGCCTTCGATTAT |
| 133 | DNA | CAGTCTGTTCTGACACAGCCTCCTAGCGCCTCTGGCACACCTGGACA |
| | | GAGAGTGACCATCAGCTGTAGCGGCAGCAGCTCCAACATCGGCAGC |
| | | AACACCGTGAACTGGTATCAGCAGCTGCCTGGCACAGCCCCTAAACT |
| | | GCTGATCTACTACGACGACCTGCGGCCTAGCGGCGTGCCAGATAGAT |
| | | TTTCTGGCAGCAAGAGCGGCACCTCTGCCAGCCTGGCTATTTCTGGA |
| | | CTGCAGAGCGAGGACGAGGCCGACTATTATTGTGCCGCCTGGGACGA |
| | | CAGCCTGAACGGCTATGTTGTTTTCGGCGGAGGCACCAAGCTGACCG |
| | | TTCTA |
| 134 | DNA | AGCGGCAGCAGCTCCAACATCGGCAGCAACACCGTGAAC |
| 135 | DNA | TACGACGACCTGCGGCCTAGC |
| 136 | DNA | GCCGCCTGGGACGACAGCCTGAACGGCTATGTTGTT |
| 137 | PRT | EVQLLESGGGLVQPGGSLRLSCAASGFTFSSYAMSWVRQAPGKGLEWV |
| | | SAIGTGGDTYYADSVKGRFTISRDNSKNTLYLQMNSLRAEDTAVYYCA |
| | | RRDDYTSRDAFDYWGQGTLVTVSSASTKGPSVFPLAPSSKSTSGGTAAL |
| | | GCLVKDYFPEPVTVSWNSGALTSGVHTFPAVLQSSGLYSLSSVVTVPSSS |
| | | LGTQTYICNVNHKPSNTKVDKKVEPKSCDKTHTCPPCPAPELLGGPSVFL |
| | | FPPKPKDTLMISRTPEVTCVVVDVSHEDPEVKFNWYVDGVEVHNAKTK |
| | | PREEQYNSTYRVVSVLTVLHQDWLNGKEYKCKVSNKALPAPIEKTISKA |
| | | KGQPREPQVYTLPPSRDELTKNQVSLTCLVKGFYPSDIAVEWESNGQPE |
| | | NNYKTTPPVLDSDGSFFLYSKLTVDKSRWQQGNVFSCSVMHEALHNHY |
| | | TQKSLSLSPG |
| 138 | PRT | QSVLTQPPSASGTPGQRVTISCSGSSSNIGSNTVNWYQQLPGTAPKLLIYY |
| | | DDLRPSGVPDRFSGSKSGTSASLAISGLQSEDEADYYCAAWDDSLNGYV |
| | | VFGGGTKLTVLGQPKAAPSVTLFPPSSEELQANKATLVCLISDFYPGAVT |
| | | VAWKADSSPVKAGVETTTPSKQSNNKYAASSYLSLTPEQWKSHRSYSC |
| | | QVTHEGSTVEKTVAPTECS |
| 139 | DNA | GAAGTTCAGCTGCTGGAATCTGGCGGCGGACTGGTTCAACCTGGCGG |
| | | ATCTCTGAGACTGAGCTGTGCCGCCAGCGGCTTCACCTTTAGCAGCT |
| | | ACGCCATGAGCTGGGTCCGACAGGCTCCTGGCAAAGGCCTTGAATGG |
| | | GTGTCCGCCATTGGCACAGGCGGCGATACCTACTACGCCGACTCTGT |
| | | GAAGGGCAGATTCACCATCAGCCGGGACAACAGCAAGAACACCCTG |
| | | TACCTGCAGATGAACAGCCTGAGAGCCGAGGACACCGCCGTGTACTA |
| | | TTGCGCCAGAAGGGACGACTACACCAGCAGGGACGCCTTCGATTATT |
| | | GGGGCCAGGGCACACTGGTCACCGTTTCTTCAGCCAGCACCAAGGGC |
| | | CCCAGCGTGTTCCCTCTGGCCCCTAGCAGCAAGAGCACATCTGGCGG |
| | | AACAGCCGCCCTGGGCTGCCTCGTGAAGGACTACTTTCCCGAGCCCG |
| | | TGACCGTGTCCTGGAACTCTGGCGCTCTGACAAGCGGCGTGCACACC |
| | | TTTCCAGCCGTGCTGCAGAGCAGCGGCCTGTACTCTCTGAGCAGCGT |
| | | CGTGACAGTGCCCAGCAGCTCTCTGGGCACCCAGACCTACATCTGCA |
| | | ACGTGAACCACAAGCCCAGCAACACCAAGGTGGACAAGAAGGTGGA |
| | | ACCCAAGAGCTGCGACAAGACCCACACCTGTCCCCCTTGTCCTGCCC |
| | | CCGAACTGCTGGGAGGCCCTTCCGTGTTCCTGTTCCCCCCAAAGCCCA |
| | | AGGACACCCTGATGATCAGCCGGACCCCCGAAGTGACCTGCGTGGTG |
| | | GTGGATGTGTCCCACGAGGACCCTGAAGTGAAGTTCAATTGGTACGT |
| | | GGACGGCGTGGAAGTGCACAACGCCAAGACCAAGCCTAGAGAGGAA |
| | | CAGTACAACAGCACCTACCGGGTGGTGTCCGTGCTGACAGTGCTGCA |
| | | CCAGGACTGGCTGAACGGCAAAGAGTACAAGTGCAAGGTGTCCAAC |
| | | AAGGCCCTGCCTGCCCCCATCGAGAAAACCATCAGCAAGGCCAAGG |
| | | GCCAGCCCCGCGAACCCCAGGTGTACACACTGCCCCCAAGCAGGGAC |
| | | GAGCTGACCAAGAACCAGGTGTCCCTGACCTGTCTCGTGAAAGGCTT |
| | | CTACCCCTCCGATATCGCCGTGGAATGGGAGAGCAACGGCCAGCCCG |
| | | AGAACAACTACAAGACCACCCCCCCTGTGCTGGACAGCGACGGCTCA |
| | | TTCTTCCTGTACAGCAAGCTGACCGTGGACAAGTCCCGGTGGCAGCA |
| | | GGGCAACGTGTTCAGCTGCAGCGTGATGCACGAGGCCCTGCACAACC |
| | | ACTACACCCAGAAGTCCCTGAGCCTGAGCCCTGGC |
| 140 | DNA | CAGTCTGTTCTGACACAGCCTCCTAGCGCCTCTGGCACACCTGGACA |
| | | GAGAGTGACCATCAGCTGTAGCGGCAGCAGCTCCAACATCGGCAGC |
| | | AACACCGTGAACTGGTATCAGCAGCTGCCTGGCACAGCCCCTAAACT |
| | | GCTGATCTACTACGACGACCTGCGGCCTAGCGGCGTGCCAGATAGAT |
| | | TTTCTGGCAGCAAGAGCGGCACCTCTGCCAGCCTGGCTATTTCTGGA |
| | | CTGCAGAGCGAGGACGAGGCCGACTATTATTGTGCCGCCTGGGACGA |
| | | CAGCCTGAACGGCTATGTTGTTTTCGGCGGAGGCACCAAGCTGACCG |
| | | TTCTAGGCCAGCCTAAAGCCGCCCCTAGCGTGACCCTGTTCCCTCCAA |
| | | GCAGCGAGGAACTGCAGGCCAACAAGGCCACCCTCGTGTGCCTGATC |
| | | AGCGACTTCTATCCTGGCGCCGTGACCGTGGCCTGGAAGGCCGATAG |
| | | CTCTCCTGTGAAGGCCGGCGTGGAAACCACCACCCCTAGCAAGCAGA |
| | | GCAACAACAAATACGCCGCCAGCAGCTACCTGAGCCTGACCCCCGAG |
| | | CAGTGGAAGTCCCACAGATCCTACAGCTGCCAAGTGACCCACGAGGG |
| | | CAGCACCGTGGAAAAGACAGTGGCCCCTACCGAGTGCAGC |
| 141 | PRT | EVQLLESGGGLVQPGGSLRLSCAASGFTFYSYAMSWVRQAPGKGLEWV |
| | | SAIGTGGDTYYADSVKGRFTISRDNSKNTLYLQMNSLRAEDTAVYYCA |
| | | RRDDYTSRDAFDYWGQGTLVTVSS |
| 142 | PRT | SYAMS |
| 143 | PRT | AIGTGGDTYYADSVKG |
| 144 | PRT | RDDYTSRDAFDY |
| 145 | PRT | QSVLTQPPSASGTPGQRVTISCSGSSSNIGSNTVNWYQQLPGTAPKLLIYY |
| | | DDLRPSGVPDRFSGSKSGTSASLAISGLQSEDEADYYCAAWDDSLNDYV |
| | | VFGGGTKLTVL |
| 146 | PRT | SGSSSNIGSNTVN |
| 147 | PRT | YDDLRPS |
| 148 | PRT | AAWDDSLNDYVV |
| 149 | DNA | GAAGTTCAGCTGCTGGAATCTGGCGGCGGACTGGTTCAACCTGGCGG |
| | | ATCTCTGAGACTGAGCTGTGCCGCCAGCGGCTTCACCTTTTACAGCTA |
| | | CGCCATGAGCTGGGTCCGACAGGCCCCTGGAAAAGGCCTTGAATGGG |
| | | TGTCCGCCATCGGCACAGGCGGCGATACCTACTATGCCGACTCTGTG |
| | | AAGGGCAGATTCACCATCAGCCGGGACAACAGCAAGAACACCCTGT |
| | | ACCTGCAGATGAACAGCCTGAGAGCCGAGGACACCGCCGTGTACTAT |
| | | TGCGCCAGAAGGGACGACTACACCAGCAGGGACGCCTTCGATTATTG |
| | | GGGCCAGGGCACACTGGTCACCGTTTCTTCA |
| 150 | DNA | AGCTACGCCATGAGC |
| 151 | DNA | GCCATCGGCACAGGCGGCGATACCTACTATGCCGACTCTGTGAAGGG |
| | | C |
| 152 | DNA | AGGGACGACTACACCAGCAGGGACGCCTTCGATTAT |
| 153 | DNA | CAGTCTGTTCTGACACAGCCTCCTAGCGCCTCTGGCACACCTGGACA |
| | | GAGAGTGACCATCAGCTGTAGCGGCAGCAGCTCCAACATCGGCAGC |
| | | AACACCGTGAACTGGTATCAGCAGCTGCCTGGCACAGCCCCTAAACT |
| | | GCTGATCTACTACGACGACCTGCGGCCTAGCGGCGTGCCAGATAGAT |
| | | TTTCTGGCAGCAAGAGCGGCACCTCTGCCAGCCTGGCTATTTCTGGA |
| | | CTGCAGAGCGAGGACGAGGCCGACTATTATTGTGCCGCCTGGGACGA |
| | | CAGCCTGAACGACTACGTTGTGTTTGGCGGAGGCACCAAGCTGACCG |
| | | TTCTA |
| 154 | DNA | AGCGGCAGCAGCTCCAACATCGGCAGCAACACCGTGAAC |
| 155 | DNA | TACGACGACCTGCGGCCTAGC |
| 156 | DNA | GCCGCCTGGGACGACAGCCTGAACGACTACGTTGTG |
| 157 | PRT | EVQLLESGGGLVQPGGSLRLSCAASGFTFYSYAMSWVRQAPGKGLEWV |
| | | SAIGTGGDTYYADSVKGRFTISRDNSKNTLYLQMNSLRAEDTAVYYCA |
| | | RRDDYTSRDAFDYWGQGTLVTVSSASTKGPSVFPLAPCSRSTSESTAAL |
| | | GCLVKDYFPEPVTVSWNSGALTSGVHTFPAVLQSSGLYSLSSVVTVPSSS |
| | | LGTKTYTCNVDHKPSNTKVDKRVESKYGPPCPPCPAPEFLGGPSVFLFPP |
| | | KPKDTLMISRTPEVTCVVVDVSQEDPEVQFNWYVDGVEVHNAKTKPRE |
| | | EQFNSTYRVVSVLTVLHQDWLNGKEYKCKVSNKGLPSSIEKTISKAKGQ |
| | | PREPQVYTLPPSQEEMTKNQVSLTCLVKGFYPSDIAVEWESNGQPENNY |
| | | KTTPPVLDSDGSFFLYSRLTVDKSRWQEGNVFSCSVMHEALHNHYTQK |
| | | SLSLSLGK |
| 158 | PRT | QSVLTQPPSASGTPGQRVTISCSGSSSNIGSNTVNWYQQLPGTAPKLLIYY |
| | | DDLRPSGVPDRFSGSKSGTSASLAISGLQSEDEADYYCAAWDDSLNDYV |
| | | VFGGGTKLTVLGQPKAAPSVTLFPPSSEELQANKATLVCLISDFYPGAVT |
| | | VAWKADSSPVKAGVETTTPSKQSNNKYAASSYLSLTPEQWKSHRSYSC |
| | | QVTHEGSTVEKTVAPTECS |
| 159 | DNA | GAAGTTCAGCTGCTGGAATCTGGCGGCGGACTGGTTCAACCTGGCGG |
| | | ATCTCTGAGACTGAGCTGTGCCGCCAGCGGCTTCACCTTTTACAGCTA |
| | | CGCCATGAGCTGGGTCCGACAGGCCCCTGGAAAAGGCCTTGAATGGG |
| | | TGTCCGCCATCGGCACAGGCGGCGATACCTACTATGCCGACTCTGTG |
| | | AAGGGCAGATTCACCATCAGCCGGGACAACAGCAAGAACACCCTGT |
| | | ACCTGCAGATGAACAGCCTGAGAGCCGAGGACACCGCCGTGTACTAT |
| | | TGCGCCAGAAGGGACGACTACACCAGCAGGGACGCCTTCGATTATTG |
| | | GGGCCAGGGCACACTGGTCACCGTTTCTTCAGCCAGCACCAAGGGCC |
| | | CCAGCGTGTTCCCTCTGGCCCCTTGTAGCAGAAGCACCAGCGAGTCT |
| | | ACAGCCGCCCTGGGCTGCCTCGTGAAGGACTACTTTCCCGAGCCCGT |
| | | GACCGTGTCCTGGAACTCTGGCGCTCTGACAAGCGGCGTGCACACCT |
| | | TTCCAGCCGTGCTGCAGAGCAGCGGCCTGTACTCTCTGAGCAGCGTC |
| | | GTGACAGTGCCCAGCAGCAGCCTGGGCACCAAGACCTACACCTGTAA |
| | | CGTGGACCACAAGCCCAGCAACACCAAGGTGGACAAGCGGGTGGAA |
| | | TCTAAGTACGGCCCTCCCTGCCCTCCTTGCCCAGCCCCTGAATTTCTG |
| | | GGCGGACCCTCCGTGTTCCTGTTCCCCCCAAAGCCCAAGGACACCCT |
| | | GATGATCAGCCGGACCCCCGAAGTGACCTGCGTGGTGGTGGATGTGT |
| | | CCCAGGAAGATCCCGAGGTGCAGTTCAATTGGTACGTGGACGGCGTG |
| | | GAAGTGCACAACGCCAAGACCAAGCCCAGAGAGGAACAGTTCAACA |
| | | GCACCTACCGGGTGGTGTCCGTGCTGACAGTGCTGCACCAGGACTGG |
| | | CTGAACGGCAAAGAGTACAAGTGCAAGGTGTCCAACAAGGGCCTGC |
| | | CCAGCTCCATCGAGAAAACCATCAGCAAGGCCAAGGGCCAGCCCCG |
| | | CGAACCCCAGGTGTACACACTGCCTCCAAGCCAGGAAGAGATGACC |
| | | AAGAACCAGGTGTCCCTGACCTGTCTCGTGAAAGGCTTCTACCCCTC |
| | | CGATATCGCCGTGGAATGGGAGAGCAACGGCCAGCCCGAGAACAAC |
| | | TACAAGACCACCCCCCCTGTGCTGGACAGCGACGGCTCATTCTTCCT |
| | | GTACAGCAGACTGACCGTGGACAAGAGCCGGTGGCAGGAAGGCAAC |
| | | GTGTTCAGCTGCAGCGTGATGCACGAGGCCCTGCACAACCACTACAC |
| | | CCAGAAGTCCCTGTCTCTGAGCCTGGGCAAG |
| 160 | DNA | CAGTCTGTTCTGACACAGCCTCCTAGCGCCTCTGGCACACCTGGACA |
| | | GAGAGTGACCATCAGCTGTAGCGGCAGCAGCTCCAACATCGGCAGC |
| | | AACACCGTGAACTGGTATCAGCAGCTGCCTGGCACAGCCCCTAAACT |
| | | GCTGATCTACTACGACGACCTGCGGCCTAGCGGCGTGCCAGATAGAT |
| | | TTTCTGGCAGCAAGAGCGGCACCTCTGCCAGCCTGGCTATTTCTGGA |
| | | CTGCAGAGCGAGGACGAGGCCGACTATTATTGTGCCGCCTGGGACGA |
| | | CAGCCTGAACGACTACGTTGTGTTTGGCGGAGGCACCAAGCTGACCG |
| | | TTCTAGGCCAGCCTAAAGCCGCCCCTAGCGTGACCCTGTTCCCTCCAA |
| | | GCAGCGAGGAACTGCAGGCCAACAAGGCCACCCTCGTGTGCCTGATC |
| | | AGCGACTTCTATCCTGGCGCCGTGACCGTGGCCTGGAAGGCCGATAG |
| | | CTCTCCTGTGAAGGCCGGCGTGGAAACCACCACCCCTAGCAAGCAGA |
| | | GCAACAACAAATACGCCGCCAGCAGCTACCTGAGCCTGACCCCCGAG |
| | | CAGTGGAAGTCCCACAGATCCTACAGCTGCCAAGTGACCCACGAGGG |
| | | CAGCACCGTGGAAAAGACAGTGGCCCCTACCGAGTGCAGC |
| 161 | PRT | EVQLLESGGGLVQPGGSLRLSCAASGFTFSSYAMSWVRQAPGKGLEWV |
| | | SAIGYGGDTYYADSVKGRFTISRDNSKNTLYLQMNSLRAEDTAVYYCA |
| | | RRDDYTSRDAFDYWGQGTLVTVSS |
| 162 | PRT | SYAMS |
| 163 | PRT | AIGYGGDTYYADSVKG |
| 164 | PRT | RDDYTSRDAFDY |
| 165 | PRT | QSVLTQPPSASGTPGQRVTISCSGSSSNIGSNTVNWYQQLPGTAPKLLIYY |
| | | DDLRPSGVPDRFSGSKSGTSASLAISGLQSEDEADYYCAAWDDSLNDIV |
| | | VFGGGTKLTVL |
| 166 | PRT | SGSSSNIGSNTVN |
| 167 | PRT | YDDLRPS |
| 168 | PRT | AAWDDSLNDIVV |
| 169 | DNA | GAAGTTCAGCTGCTGGAATCTGGCGGCGGACTGGTTCAACCTGGCGG |
| | | ATCTCTGAGACTGAGCTGTGCCGCCAGCGGCTTCACCTTTAGCAGCT |
| | | ACGCCATGAGCTGGGTCCGACAGGCTCCTGGCAAAGGCCTTGAATGG |
| | | GTGTCCGCCATCGGCTATGGCGGCGATACCTACTACGCCGACTCTGT |
| | | GAAGGGCAGATTCACCATCAGCCGGGACAACAGCAAGAACACCCTG |
| | | TACCTGCAGATGAACAGCCTGAGAGCCGAGGACACCGCCGTGTACTA |
| | | TTGCGCCAGAAGGGACGACTACACCAGCAGGGACGCCTTCGATTATT |
| | | GGGGCCAGGGCACACTGGTCACCGTTTCTTCA |
| 170 | DNA | AGCTACGCCATGAGC |
| 171 | DNA | GCCATCGGCTATGGCGGCGATACCTACTACGCCGACTCTGTGAAGGG |
| | | C |
| 172 | DNA | AGGGACGACTACACCAGCAGGGACGCCTTCGATTAT |
| 173 | DNA | CAGTCTGTTCTGACACAGCCTCCTAGCGCCTCTGGCACACCTGGACA |
| | | GAGAGTGACCATCAGCTGTAGCGGCAGCAGCTCCAACATCGGCAGC |
| | | AACACCGTGAACTGGTATCAGCAGCTGCCTGGCACAGCCCCTAAACT |
| | | GCTGATCTACTACGACGACCTGCGGCCTAGCGGCGTGCCAGATAGAT |
| | | TTTCTGGCAGCAAGAGCGGCACCTCTGCCAGCCTGGCTATTTCTGGA |
| | | CTGCAGAGCGAGGACGAGGCCGACTATTATTGTGCCGCCTGGGACGA |
| | | CAGCCTGAACGACATCGTTGTTTTCGGCGGAGGCACCAAGCTGACCG |
| | | TTCTA |
| 174 | DNA | AGCGGCAGCAGCTCCAACATCGGCAGCAACACCGTGAAC |
| 175 | DNA | TACGACGACCTGCGGCCTAGC |
| 176 | DNA | GCCGCCTGGGACGACAGCCTGAACGACATCGTTGTT |
| 177 | PRT | EVQLLESGGGLVQPGGSLRLSCAASGFTFSSYAMSWVRQAPGKGLEWV |
| | | SAIGYGGDTYYADSVKGRFTISRDNSKNTLYLQMNSLRAEDTAVYYCA |
| | | RRDDYTSRDAFDYWGQGTLVTVSSASTKGPSVFPLAPCSRSTSESTAAL |
| | | GCLVKDYFPEPVTVSWNSGALTSGVHTFPAVLQSSGLYSLSSVVTVPSSS |
| | | LGTKTYTCNVDHKPSNTKVDKRVESKYGPPCPPCPAPEFLGGPSVFLFPP |
| | | KPKDTLMISRTPEVTCVVVDVSQEDPEVQFNWYVDGVEVHNAKTKPRE |
| | | EQFNSTYRVVSVLTVLHQDWLNGKEYKCKVSNKGLPSSIEKTISKAKGQ |
| | | PREPQVYTLPPSQEEMTKNQVSLTCLVKGFYPSDIAVEWESNGQPENNY |
| | | KTTPPVLDSDGSFFLYSRLTVDKSRWQEGNVFSCSVMHEALHNHYTQK |
| | | SLSLSLGK |
| 178 | PRT | QSVLTQPPSASGTPGQRVTISCSGSSSNIGSNTVNWYQQLPGTAPKLLIYY |
| | | DDLRPSGVPDRFSGSKSGTSASLAISGLQSEDEADYYCAAWDDSLNDIV |
| | | VFGGGTKLTVLGQPKAAPSVTLFPPSSEELQANKATLVCLISDFYPGAVT |
| | | VAWKADSSPVKAGVETTTPSKQSNNKYAASSYLSLTPEQWKSHRSYSC |
| | | QVTHEGSTVEKTVAPTECS |
| 179 | DNA | GAAGTTCAGCTGCTGGAATCTGGCGGCGGACTGGTTCAACCTGGCGG |
| | | ATCTCTGAGACTGAGCTGTGCCGCCAGCGGCTTCACCTTTAGCAGCT |
| | | ACGCCATGAGCTGGGTCCGACAGGCTCCTGGCAAAGGCCTTGAATGG |
| | | GTGTCCGCCATCGGCTATGGCGGCGATACCTACTACGCCGACTCTGT |
| | | GAAGGGCAGATTCACCATCAGCCGGGACAACAGCAAGAACACCCTG |
| | | TACCTGCAGATGAACAGCCTGAGAGCCGAGGACACCGCCGTGTACTA |
| | | TTGCGCCAGAAGGGACGACTACACCAGCAGGGACGCCTTCGATTATT |
| | | GGGGCCAGGGCACACTGGTCACCGTTTCTTCAGCCAGCACCAAGGGC |
| | | CCCAGCGTGTTCCCTCTGGCCCCTTGTAGCAGAAGCACCAGCGAGTC |
| | | TACAGCCGCCCTGGGCTGCCTCGTGAAGGACTACTTTCCCGAGCCCG |
| | | TGACCGTGTCCTGGAACTCTGGCGCTCTGACAAGCGGCGTGCACACC |
| | | TTTCCAGCCGTGCTGCAGAGCAGCGGCCTGTACTCTCTGAGCAGCGT |
| | | CGTGACAGTGCCCAGCAGCAGCCTGGGCACCAAGACCTACACCTGTA |
| | | ACGTGGACCACAAGCCCAGCAACACCAAGGTGGACAAGCGGGTGGA |
| | | ATCTAAGTACGGCCCTCCCTGCCCTCCTTGCCCAGCCCCTGAATTTCT |
| | | GGGCGGACCCTCCGTGTTCCTGTTCCCCCCAAAGCCCAAGGACACCC |
| | | TGATGATCAGCCGGACCCCCGAAGTGACCTGCGTGGTGGTGGATGTG |
| | | TCCCAGGAAGATCCCGAGGTGCAGTTCAATTGGTACGTGGACGGCGT |
| | | GGAAGTGCACAACGCCAAGACCAAGCCCAGAGAGGAACAGTTCAAC |
| | | AGCACCTACCGGGTGGTGTCCGTGCTGACAGTGCTGCACCAGGACTG |
| | | GCTGAACGGCAAAGAGTACAAGTGCAAGGTGTCCAACAAGGGCCTG |
| | | CCCAGCTCCATCGAGAAAACCATCAGCAAGGCCAAGGGCCAGCCCC |
| | | GCGAACCCCAGGTGTACACACTGCCTCCAAGCCAGGAAGAGATGAC |
| | | CAAGAACCAGGTGTCCCTGACCTGTCTCGTGAAAGGCTTCTACCCCT |
| | | CCGATATCGCCGTGGAATGGGAGAGCAACGGCCAGCCCGAGAACAA |
| | | CTACAAGACCACCCCCCCTGTGCTGGACAGCGACGGCTCATTCTTCCT |
| | | GTACAGCAGACTGACCGTGGACAAGAGCCGGTGGCAGGAAGGCAAC |
| | | GTGTTCAGCTGCAGCGTGATGCACGAGGCCCTGCACAACCACTACAC |
| | | CCAGAAGTCCCTGTCTCTGAGCCTGGGCAAG |
| 180 | DNA | CAGTCTGTTCTGACACAGCCTCCTAGCGCCTCTGGCACACCTGGACA |
| | | GAGAGTGACCATCAGCTGTAGCGGCAGCAGCTCCAACATCGGCAGC |
| | | AACACCGTGAACTGGTATCAGCAGCTGCCTGGCACAGCCCCTAAACT |
| | | GCTGATCTACTACGACGACCTGCGGCCTAGCGGCGTGCCAGATAGAT |
| | | TTTCTGGCAGCAAGAGCGGCACCTCTGCCAGCCTGGCTATTTCTGGA |
| | | CTGCAGAGCGAGGACGAGGCCGACTATTATTGTGCCGCCTGGGACGA |
| | | CAGCCTGAACGACATCGTTGTTTTCGGCGGAGGCACCAAGCTGACCG |
| | | TTCTAGGCCAGCCTAAAGCCGCCCCTAGCGTGACCCTGTTCCCTCCAA |
| | | GCAGCGAGGAACTGCAGGCCAACAAGGCCACCCTCGTGTGCCTGATC |
| | | AGCGACTTCTATCCTGGCGCCGTGACCGTGGCCTGGAAGGCCGATAG |
| | | CTCTCCTGTGAAGGCCGGCGTGGAAACCACCACCCCTAGCAAGCAGA |
| | | GCAACAACAAATACGCCGCCAGCAGCTACCTGAGCCTGACCCCCGAG |
| | | CAGTGGAAGTCCCACAGATCCTACAGCTGCCAAGTGACCCACGAGGG |
| | | CAGCACCGTGGAAAAGACAGTGGCCCCTACCGAGTGCAGC |
| 181 | PRT | EVQLLESGGGLVQPGGSLRLSCAASGFTFSSYAMSWVRQAPGKGLEWV |
| | | SAIGYGGDTYYADSVKGRFTISRDNSKNTLYLQMNSLRAEDTAVYYCA |
| | | RRDDYTSRDAFDYWGQGTLVTVSS |
| 182 | PRT | SYAMS |
| 183 | PRT | AIGYGGDTYYADSVKG |
| 184 | PRT | RDDYTSRDAFDY |
| 185 | PRT | QSVLTQPPSASGTPGQRVTISCSGSSSNIGSNTVNWYQQLPGTAPKLLIYY |
| | | DDLRPSGVPDRFSGSKSGTSASLAISGLQSEDEADYYCAAWDDSLNVYP |
| | | VFGGGTKLTVL |
| 186 | PRT | SGSSSNIGSNTVN |
| 187 | PRT | YDDLRPS |
| 188 | PRT | AAWDDSLNVYPV |
| 189 | DNA | GAAGTTCAGCTGCTGGAATCTGGCGGCGGACTGGTTCAACCTGGCGG |
| | | ATCTCTGAGACTGAGCTGTGCCGCCAGCGGCTTCACCTTTAGCAGCT |
| | | ACGCCATGAGCTGGGTCCGACAGGCTCCTGGCAAAGGCCTTGAATGG |
| | | GTGTCCGCCATCGGCTATGGCGGCGATACCTACTACGCCGACTCTGT |
| | | GAAGGGCAGATTCACCATCAGCCGGGACAACAGCAAGAACACCCTG |
| | | TACCTGCAGATGAACAGCCTGAGAGCCGAGGACACCGCCGTGTACTA |
| | | TTGCGCCAGAAGGGACGACTACACCAGCAGGGACGCCTTCGATTATT |
| | | GGGGCCAGGGCACACTGGTCACCGTTTCTTCA |
| 190 | DNA | AGCTACGCCATGAGC |
| 191 | DNA | GCCATCGGCTATGGCGGCGATACCTACTACGCCGACTCTGTGAAGGG |
| | | C |
| 192 | DNA | AGGGACGACTACACCAGCAGGGACGCCTTCGATTAT |
| 193 | DNA | CAGTCTGTTCTGACACAGCCTCCTAGCGCCTCTGGCACACCTGGACA |
| | | GAGAGTGACCATCAGCTGTAGCGGCAGCAGCTCCAACATCGGCAGC |
| | | AACACCGTGAACTGGTATCAGCAGCTGCCTGGCACAGCCCCTAAACT |
| | | GCTGATCTACTACGACGACCTGCGGCCTAGCGGCGTGCCAGATAGAT |
| | | TTTCTGGCAGCAAGAGCGGCACCTCTGCCAGCCTGGCTATTTCTGGA |
| | | CTGCAGAGCGAGGACGAGGCCGACTATTATTGTGCCGCCTGGGACGA |
| | | CAGCCTGAACGTGTACCCTGTTTTTGGCGGAGGCACCAAGCTGACCG |
| | | TTCTA |
| 194 | DNA | AGCGGCAGCAGCTCCAACATCGGCAGCAACACCGTGAAC |
| 195 | DNA | TACGACGACCTGCGGCCTAGC |
| 196 | DNA | GCCGCCTGGGACGACAGCCTGAACGTGTACCCTGTT |
| 197 | PRT | EVQLLESGGGLVQPGGSLRLSCAASGFTFSSYAMSWVRQAPGKGLEWV |
| | | SAIGYGGDTYYADSVKGRFTISRDNSKNTLYLQMNSLRAEDTAVYYCA |
| | | RRDDYTSRDAFDYWGQGTLVTVSSASTKGPSVFPLAPCSRSTSESTAAL |
| | | GCLVKDYFPEPVTVSWNSGALTSGVHTFPAVLQSSGLYSLSSVVTVPSSS |
| | | LGTKTYTCNVDHKPSNTKVDKRVESKYGPPCPPCPAPEFLGGPSVFLFPP |
| | | KPKDTLMISRTPEVTCVVVDVSQEDPEVQFNWYVDGVEVHNAKTKPRE |
| | | EQFNSTYRVVSVLTVLHQDWLNGKEYKCKVSNKGLPSSIEKTISKAKGQ |
| | | PREPQVYTLPPSQEEMTKNQVSLTCLVKGFYPSDIAVEWESNGQPENNY |
| | | KTTPPVLDSDGSFFLYSRLTVDKSRWQEGNVFSCSVMHEALHNHYTQK |
| | | SLSLSLGK |
| 198 | PRT | QSVLTQPPSASGTPGQRVTISCSGSSSNIGSNTVNWYQQLPGTAPKLLIYY |
| | | DDLRPSGVPDRFSGSKSGTSASLAISGLQSEDEADYYCAAWDDSLNVYP |
| | | VFGGGTKLTVLGQPKAAPSVTLFPPSSEELQANKATLVCLISDFYPGAVT |
| | | VAWKADSSPVKAGVETTTPSKQSNNKYAASSYLSLTPEQWKSHRSYSC |
| | | QVTHEGSTVEKTVAPTECS |
| 199 | DNA | GAAGTTCAGCTGCTGGAATCTGGCGGCGGACTGGTTCAACCTGGCGG |
| | | ATCTCTGAGACTGAGCTGTGCCGCCAGCGGCTTCACCTTTAGCAGCT |
| | | ACGCCATGAGCTGGGTCCGACAGGCTCCTGGCAAAGGCCTTGAATGG |
| | | GTGTCCGCCATCGGCTATGGCGGCGATACCTACTACGCCGACTCTGT |
| | | GAAGGGCAGATTCACCATCAGCCGGGACAACAGCAAGAACACCCTG |
| | | TACCTGCAGATGAACAGCCTGAGAGCCGAGGACACCGCCGTGTACTA |
| | | TTGCGCCAGAAGGGACGACTACACCAGCAGGGACGCCTTCGATTATT |
| | | GGGGCCAGGGCACACTGGTCACCGTTTCTTCAGCCAGCACCAAGGGC |
| | | CCCAGCGTGTTCCCTCTGGCCCCTTGTAGCAGAAGCACCAGCGAGTC |
| | | TACAGCCGCCCTGGGCTGCCTCGTGAAGGACTACTTTCCCGAGCCCG |
| | | TGACCGTGTCCTGGAACTCTGGCGCTCTGACAAGCGGCGTGCACACC |
| | | TTTCCAGCCGTGCTGCAGAGCAGCGGCCTGTACTCTCTGAGCAGCGT |
| | | CGTGACAGTGCCCAGCAGCAGCCTGGGCACCAAGACCTACACCTGTA |
| | | ACGTGGACCACAAGCCCAGCAACACCAAGGTGGACAAGCGGGTGGA |
| | | ATCTAAGTACGGCCCTCCCTGCCCTCCTTGCCCAGCCCCTGAATTTCT |
| | | GGGCGGACCCTCCGTGTTCCTGTTCCCCCCAAAGCCCAAGGACACCC |
| | | TGATGATCAGCCGGACCCCCGAAGTGACCTGCGTGGTGGTGGATGTG |
| | | TCCCAGGAAGATCCCGAGGTGCAGTTCAATTGGTACGTGGACGGCGT |
| | | GGAAGTGCACAACGCCAAGACCAAGCCCAGAGAGGAACAGTTCAAC |
| | | AGCACCTACCGGGTGGTGTCCGTGCTGACAGTGCTGCACCAGGACTG |
| | | GCTGAACGGCAAAGAGTACAAGTGCAAGGTGTCCAACAAGGGCCTG |
| | | CCCAGCTCCATCGAGAAAACCATCAGCAAGGCCAAGGGCCAGCCCC |
| | | GCGAACCCCAGGTGTACACACTGCCTCCAAGCCAGGAAGAGATGAC |
| | | CAAGAACCAGGTGTCCCTGACCTGTCTCGTGAAAGGCTTCTACCCCT |
| | | CCGATATCGCCGTGGAATGGGAGAGCAACGGCCAGCCCGAGAACAA |
| | | CTACAAGACCACCCCCCCTGTGCTGGACAGCGACGGCTCATTCTTCCT |
| | | GTACAGCAGACTGACCGTGGACAAGAGCCGGTGGCAGGAAGGCAAC |
| | | GTGTTCAGCTGCAGCGTGATGCACGAGGCCCTGCACAACCACTACAC |
| | | CCAGAAGTCCCTGTCTCTGAGCCTGGGCAAG |
| 200 | DNA | CAGTCTGTTCTGACACAGCCTCCTAGCGCCTCTGGCACACCTGGACA |
| | | GAGAGTGACCATCAGCTGTAGCGGCAGCAGCTCCAACATCGGCAGC |
| | | AACACCGTGAACTGGTATCAGCAGCTGCCTGGCACAGCCCCTAAACT |
| | | GCTGATCTACTACGACGACCTGCGGCCTAGCGGCGTGCCAGATAGAT |
| | | TTTCTGGCAGCAAGAGCGGCACCTCTGCCAGCCTGGCTATTTCTGGA |
| | | CTGCAGAGCGAGGACGAGGCCGACTATTATTGTGCCGCCTGGGACGA |
| | | CAGCCTGAACGTGTACCCTGTTTTTGGCGGAGGCACCAAGCTGACCG |
| | | TTCTAGGCCAGCCTAAAGCCGCCCCTAGCGTGACCCTGTTCCCTCCAA |
| | | GCAGCGAGGAACTGCAGGCCAACAAGGCCACCCTCGTGTGCCTGATC |
| | | AGCGACTTCTATCCTGGCGCCGTGACCGTGGCCTGGAAGGCCGATAG |
| | | CTCTCCTGTGAAGGCCGGCGTGGAAACCACCACCCCTAGCAAGCAGA |
| | | GCAACAACAAATACGCCGCCAGCAGCTACCTGAGCCTGACCCCCGAG |
| | | CAGTGGAAGTCCCACAGATCCTACAGCTGCCAAGTGACCCACGAGGG |
| | | CAGCACCGTGGAAAAGACAGTGGCCCCTACCGAGTGCAGC |
| 201 | PRT | EVQLLESGGGLVQPGGSLRLSCAASGFTFSSYAMSWVRQAPGKGLEWV |
| | | SAIGYGGDTYYADSVKGRFTISRDNSKNTLYLQMNSLRAEDTAVYYCA |
| | | RRDDYTSRDAFDYWGQGTLVTVSS |
| 202 | PRT | SYAMS |
| 203 | PRT | AIGYGGDTYYADSVKG |
| 204 | PRT | RDDYTSRDAFDY |
| 205 | PRT | QSVLTQPPSASGTPGQRVTISCSGSSSNIGSNTVNWYQQLPGTAPKLLIYY |
| | | DDLRPSGVPDRFSGSKSGTSASLAISGLQSEDEADYYCHAWDDSLNDIV |
| | | VFGGGTKLTVL |
| 206 | PRT | SGSSSNIGSNTVN |
| 207 | PRT | YDDLRPS |
| 208 | PRT | HAWDDSLNDIVV |
| 209 | DNA | GAAGTTCAGCTGCTGGAATCTGGCGGCGGACTGGTTCAACCTGGCGG |
| | | ATCTCTGAGACTGAGCTGTGCCGCCAGCGGCTTCACCTTTAGCAGCT |
| | | ACGCCATGAGCTGGGTCCGACAGGCTCCTGGCAAAGGCCTTGAATGG |
| | | GTGTCCGCCATCGGCTATGGCGGCGATACCTACTACGCCGACTCTGT |
| | | GAAGGGCAGATTCACCATCAGCCGGGACAACAGCAAGAACACCCTG |
| | | TACCTGCAGATGAACAGCCTGAGAGCCGAGGACACCGCCGTGTACTA |
| | | TTGCGCCAGAAGGGACGACTACACCAGCAGGGACGCCTTCGATTATT |
| | | GGGGCCAGGGCACACTGGTCACCGTTTCTTCA |
| 210 | DNA | AGCTACGCCATGAGC |
| 211 | DNA | GCCATCGGCTATGGCGGCGATACCTACTACGCCGACTCTGTGAAGGG |
| | | C |
| 212 | DNA | AGGGACGACTACACCAGCAGGGACGCCTTCGATTAT |
| 213 | DNA | CAGTCTGTTCTGACACAGCCTCCTAGCGCCTCTGGCACACCTGGACA |
| | | GAGAGTGACCATCAGCTGTAGCGGCAGCAGCTCCAACATCGGCAGC |
| | | AACACCGTGAACTGGTATCAGCAGCTGCCTGGCACAGCCCCTAAACT |
| | | GCTGATCTACTACGACGACCTGCGGCCTAGCGGCGTGCCAGATAGAT |
| | | TTTCTGGCAGCAAGAGCGGCACCTCTGCCAGCCTGGCTATTTCTGGA |
| | | CTGCAGAGCGAGGACGAGGCCGACTACTATTGTCACGCCTGGGACGA |
| | | CAGCCTGAACGACATCGTGGTTTTTGGCGGAGGCACCAAGCTGACCG |
| | | TTCTA |
| 214 | DNA | AGCGGCAGCAGCTCCAACATCGGCAGCAACACCGTGAAC |
| 215 | DNA | TACGACGACCTGCGGCCTAGC |
| 216 | DNA | CACGCCTGGGACGACAGCCTGAACGACATCGTGGTT |
| 217 | PRT | EVQLLESGGGLVQPGGSLRLSCAASGFTFSSYAMSWVRQAPGKGLEWV |
| | | SAIGYGGDTYYADSVKGRFTISRDNSKNTLYLQMNSLRAEDTAVYYCA |
| | | RRDDYTSRDAFDYWGQGTLVTVSSASTKGPSVFPLAPCSRSTSESTAAL |
| | | GCLVKDYFPEPVTVSWNSGALTSGVHTFPAVLQSSGLYSLSSVVTVPSSS |
| | | LGTKTYTCNVDHKPSNTKVDKRVESKYGPPCPPCPAPEFLGGPSVFLFPP |
| | | KPKDTLMISRTPEVTCVVVDVSQEDPEVQFNWYVDGVEVHNAKTKPRE |
| | | EQFNSTYRVVSVLTVLHQDWLNGKEYKCKVSNKGLPSSIEKTISKAKGQ |
| | | PREPQVYTLPPSQEEMTKNQVSLTCLVKGFYPSDIAVEWESNGQPENNY |
| | | KTTPPVLDSDGSFFLYSRLTVDKSRWQEGNVFSCSVMHEALHNHYTQK |
| | | SLSLSLGK |
| 218 | PRT | QSVLTQPPSASGTPGQRVTISCSGSSSNIGSNTVNWYQQLPGTAPKLLIYY |
| | | DDLRPSGVPDRFSGSKSGTSASLAISGLQSEDEADYYCHAWDDSLNDIV |
| | | VFGGGTKLTVLGQPKAAPSVTLFPPSSEELQANKATLVCLISDFYPGAVT |
| | | VAWKADSSPVKAGVETTTPSKQSNNKYAASSYLSLTPEQWKSHRSYSC |
| | | QVTHEGSTVEKTVAPTECS |
| 219 | DNA | GAAGTTCAGCTGCTGGAATCTGGCGGCGGACTGGTTCAACCTGGCGG |
| | | ATCTCTGAGACTGAGCTGTGCCGCCAGCGGCTTCACCTTTAGCAGCT |
| | | ACGCCATGAGCTGGGTCCGACAGGCTCCTGGCAAAGGCCTTGAATGG |
| | | GTGTCCGCCATCGGCTATGGCGGCGATACCTACTACGCCGACTCTGT |
| | | GAAGGGCAGATTCACCATCAGCCGGGACAACAGCAAGAACACCCTG |
| | | TACCTGCAGATGAACAGCCTGAGAGCCGAGGACACCGCCGTGTACTA |
| | | TTGCGCCAGAAGGGACGACTACACCAGCAGGGACGCCTTCGATTATT |
| | | GGGGCCAGGGCACACTGGTCACCGTTTCTTCAGCCAGCACCAAGGGC |
| | | CCCAGCGTGTTCCCTCTGGCCCCTTGTAGCAGAAGCACCAGCGAGTC |
| | | TACAGCCGCCCTGGGCTGCCTCGTGAAGGACTACTTTCCCGAGCCCG |
| | | TGACCGTGTCCTGGAACTCTGGCGCTCTGACAAGCGGCGTGCACACC |
| | | TTTCCAGCCGTGCTGCAGAGCAGCGGCCTGTACTCTCTGAGCAGCGT |
| | | CGTGACAGTGCCCAGCAGCAGCCTGGGCACCAAGACCTACACCTGTA |
| | | ACGTGGACCACAAGCCCAGCAACACCAAGGTGGACAAGCGGGTGGA |
| | | ATCTAAGTACGGCCCTCCCTGCCCTCCTTGCCCAGCCCCTGAATTTCT |
| | | GGGCGGACCCTCCGTGTTCCTGTTCCCCCCAAAGCCCAAGGACACCC |
| | | TGATGATCAGCCGGACCCCCGAAGTGACCTGCGTGGTGGTGGATGTG |
| | | TCCCAGGAAGATCCCGAGGTGCAGTTCAATTGGTACGTGGACGGCGT |
| | | GGAAGTGCACAACGCCAAGACCAAGCCCAGAGAGGAACAGTTCAAC |
| | | AGCACCTACCGGGTGGTGTCCGTGCTGACAGTGCTGCACCAGGACTG |
| | | GCTGAACGGCAAAGAGTACAAGTGCAAGGTGTCCAACAAGGGCCTG |
| | | CCCAGCTCCATCGAGAAAACCATCAGCAAGGCCAAGGGCCAGCCCC |
| | | GCGAACCCCAGGTGTACACACTGCCTCCAAGCCAGGAAGAGATGAC |
| | | CAAGAACCAGGTGTCCCTGACCTGTCTCGTGAAAGGCTTCTACCCCT |
| | | CCGATATCGCCGTGGAATGGGAGAGCAACGGCCAGCCCGAGAACAA |
| | | CTACAAGACCACCCCCCCTGTGCTGGACAGCGACGGCTCATTCTTCCT |
| | | GTACAGCAGACTGACCGTGGACAAGAGCCGGTGGCAGGAAGGCAAC |
| | | GTGTTCAGCTGCAGCGTGATGCACGAGGCCCTGCACAACCACTACAC |
| | | CCAGAAGTCCCTGTCTCTGAGCCTGGGCAAG |
| 220 | DNA | CAGTCTGTTCTGACACAGCCTCCTAGCGCCTCTGGCACACCTGGACA |
| | | GAGAGTGACCATCAGCTGTAGCGGCAGCAGCTCCAACATCGGCAGC |
| | | AACACCGTGAACTGGTATCAGCAGCTGCCTGGCACAGCCCCTAAACT |
| | | GCTGATCTACTACGACGACCTGCGGCCTAGCGGCGTGCCAGATAGAT |
| | | TTTCTGGCAGCAAGAGCGGCACCTCTGCCAGCCTGGCTATTTCTGGA |
| | | CTGCAGAGCGAGGACGAGGCCGACTACTATTGTCACGCCTGGGACGA |
| | | CAGCCTGAACGACATCGTGGTTTTTGGCGGAGGCACCAAGCTGACCG |
| | | TTCTAGGCCAGCCTAAAGCCGCCCCTAGCGTGACCCTGTTCCCTCCAA |
| | | GCAGCGAGGAACTGCAGGCCAACAAGGCCACCCTCGTGTGCCTGATC |
| | | AGCGACTTCTATCCTGGCGCCGTGACCGTGGCCTGGAAGGCCGATAG |
| | | CTCTCCTGTGAAGGCCGGCGTGGAAACCACCACCCCTAGCAAGCAGA |
| | | GCAACAACAAATACGCCGCCAGCAGCTACCTGAGCCTGACCCCCGAG |
| | | CAGTGGAAGTCCCACAGATCCTACAGCTGCCAAGTGACCCACGAGGG |
| | | CAGCACCGTGGAAAAGACAGTGGCCCCTACCGAGTGCAGC |
| 221 | PRT | EVQLLESGGGLVQPGGSLRLSCAASGFTFSSYAMSWVRQAPGKGLEWV |
| | | SAIGYGGDTYYADSVKGRFTISRDNSKNTLYLQMNSLRAEDTAVYYCA |
| | | RRDDYTSRDAFDYWGQGTLVTVSS |
| 222 | PRT | SYAMS |
| 223 | PRT | AIGYGGDTYYADSVKG |
| 224 | PRT | RDDYTSRDAFDY |
| 225 | PRT | QSVLTQPPSASGTPGQRVTISCSGSSSNIGSNTVNWYQQLPGTAPKLLIYY |
| | | DDLRPSGVPDRFSGSKSGTSASLAISGLQSEDEADYYCHAWDDSLNDYP |
| | | VFGGGTKLTVL |
| 226 | PRT | SGSSSNIGSNTVN |
| 227 | PRT | YDDLRPS |
| 228 | PRT | HAWDDSLNDYPV |
| 229 | DNA | GAAGTTCAGCTGCTGGAATCTGGCGGCGGACTGGTTCAACCTGGCGG |
| | | ATCTCTGAGACTGAGCTGTGCCGCCAGCGGCTTCACCTTTAGCAGCT |
| | | ACGCCATGAGCTGGGTCCGACAGGCTCCTGGCAAAGGCCTTGAATGG |
| | | GTGTCCGCCATCGGCTATGGCGGCGATACCTACTACGCCGACTCTGT |
| | | GAAGGGCAGATTCACCATCAGCCGGGACAACAGCAAGAACACCCTG |
| | | TACCTGCAGATGAACAGCCTGAGAGCCGAGGACACCGCCGTGTACTA |
| | | TTGCGCCAGAAGGGACGACTACACCAGCAGGGACGCCTTCGATTATT |
| | | GGGGCCAGGGCACACTGGTCACCGTTTCTTCA |
| 230 | DNA | AGCTACGCCATGAGC |
| 231 | DNA | GCCATCGGCTATGGCGGCGATACCTACTACGCCGACTCTGTGAAGGG |
| | | C |
| 232 | DNA | AGGGACGACTACACCAGCAGGGACGCCTTCGATTAT |
| 233 | DNA | CAGTCTGTTCTGACACAGCCTCCTAGCGCCTCTGGCACACCTGGACA |
| | | GAGAGTGACCATCAGCTGTAGCGGCAGCAGCTCCAACATCGGCAGC |
| | | AACACCGTGAACTGGTATCAGCAGCTGCCTGGCACAGCCCCTAAACT |
| | | GCTGATCTACTACGACGACCTGCGGCCTAGCGGCGTGCCAGATAGAT |
| | | TTTCTGGCAGCAAGAGCGGCACCTCTGCCAGCCTGGCTATTTCTGGA |
| | | CTGCAGAGCGAGGACGAGGCCGACTACTATTGTCACGCCTGGGACGA |
| | | CAGCCTGAACGACTACCCTGTTTTTGGCGGAGGCACCAAGCTGACCG |
| | | TTCTA |
| 234 | DNA | AGCGGCAGCAGCTCCAACATCGGCAGCAACACCGTGAAC |
| 235 | DNA | TACGACGACCTGCGGCCTAGC |
| 236 | DNA | CACGCCTGGGACGACAGCCTGAACGACTACCCTGTT |
| 237 | PRT | EVQLLESGGGLVQPGGSLRLSCAASGFTFSSYAMSWVRQAPGKGLEWV |
| | | SAIGYGGDTYYADSVKGRFTISRDNSKNTLYLQMNSLRAEDTAVYYCA |
| | | RRDDYTSRDAFDYWGQGTLVTVSSASTKGPSVFPLAPCSRSTSESTAAL |
| | | GCLVKDYFPEPVTVSWNSGALTSGVHTFPAVLQSSGLYSLSSVVTVPSSS |
| | | LGTKTYTCNVDHKPSNTKVDKRVESKYGPPCPPCPAPEFLGGPSVFLFPP |
| | | KPKDTLMISRTPEVTCVVVDVSQEDPEVQFNWYVDGVEVHNAKTKPRE |
| | | EQFNSTYRVVSVLTVLHQDWLNGKEYKCKVSNKGLPSSIEKTISKAKGQ |
| | | PREPQVYTLPPSQEEMTKNQVSLTCLVKGFYPSDIAVEWESNGQPENNY |
| | | KTTPPVLDSDGSFFLYSRLTVDKSRWQEGNVFSCSVMHEALHNHYTQK |
| | | SLSLSLGK |
| 238 | PRT | QSVLTQPPSASGTPGQRVTISCSGSSSNIGSNTVNWYQQLPGTAPKLLIYY |
| | | DDLRPSGVPDRFSGSKSGTSASLAISGLQSEDEADYYCHAWDDSLNDYP |
| | | VFGGGTKLTVLGQPKAAPSVTLFPPSSEELQANKATLVCLISDFYPGAVT |
| | | VAWKADSSPVKAGVETTTPSKQSNNKYAASSYLSLTPEQWKSHRSYSC |
| | | QVTHEGSTVEKTVAPTECS |
| 239 | DNA | GAAGTTCAGCTGCTGGAATCTGGCGGCGGACTGGTTCAACCTGGCGG |
| | | ATCTCTGAGACTGAGCTGTGCCGCCAGCGGCTTCACCTTTAGCAGCT |
| | | ACGCCATGAGCTGGGTCCGACAGGCTCCTGGCAAAGGCCTTGAATGG |
| | | GTGTCCGCCATCGGCTATGGCGGCGATACCTACTACGCCGACTCTGT |
| | | GAAGGGCAGATTCACCATCAGCCGGGACAACAGCAAGAACACCCTG |
| | | TACCTGCAGATGAACAGCCTGAGAGCCGAGGACACCGCCGTGTACTA |
| | | TTGCGCCAGAAGGGACGACTACACCAGCAGGGACGCCTTCGATTATT |
| | | GGGGCCAGGGCACACTGGTCACCGTTTCTTCAGCCAGCACCAAGGGC |
| | | CCCAGCGTGTTCCCTCTGGCCCCTTGTAGCAGAAGCACCAGCGAGTC |
| | | TACAGCCGCCCTGGGCTGCCTCGTGAAGGACTACTTTCCCGAGCCCG |
| | | TGACCGTGTCCTGGAACTCTGGCGCTCTGACAAGCGGCGTGCACACC |
| | | TTTCCAGCCGTGCTGCAGAGCAGCGGCCTGTACTCTCTGAGCAGCGT |
| | | CGTGACAGTGCCCAGCAGCAGCCTGGGCACCAAGACCTACACCTGTA |
| | | ACGTGGACCACAAGCCCAGCAACACCAAGGTGGACAAGCGGGTGGA |
| | | ATCTAAGTACGGCCCTCCCTGCCCTCCTTGCCCAGCCCCTGAATTTCT |
| | | GGGCGGACCCTCCGTGTTCCTGTTCCCCCCAAAGCCCAAGGACACCC |
| | | TGATGATCAGCCGGACCCCCGAAGTGACCTGCGTGGTGGTGGATGTG |
| | | TCCCAGGAAGATCCCGAGGTGCAGTTCAATTGGTACGTGGACGGCGT |
| | | GGAAGTGCACAACGCCAAGACCAAGCCCAGAGAGGAACAGTTCAAC |
| | | AGCACCTACCGGGTGGTGTCCGTGCTGACAGTGCTGCACCAGGACTG |
| | | GCTGAACGGCAAAGAGTACAAGTGCAAGGTGTCCAACAAGGGCCTG |
| | | CCCAGCTCCATCGAGAAAACCATCAGCAAGGCCAAGGGCCAGCCCC |
| | | GCGAACCCCAGGTGTACACACTGCCTCCAAGCCAGGAAGAGATGAC |
| | | CAAGAACCAGGTGTCCCTGACCTGTCTCGTGAAAGGCTTCTACCCCT |
| | | CCGATATCGCCGTGGAATGGGAGAGCAACGGCCAGCCCGAGAACAA |
| | | CTACAAGACCACCCCCCCTGTGCTGGACAGCGACGGCTCATTCTTCCT |
| | | GTACAGCAGACTGACCGTGGACAAGAGCCGGTGGCAGGAAGGCAAC |
| | | GTGTTCAGCTGCAGCGTGATGCACGAGGCCCTGCACAACCACTACAC |
| | | CCAGAAGTCCCTGTCTCTGAGCCTGGGCAAG |
| 240 | DNA | CAGTCTGTTCTGACACAGCCTCCTAGCGCCTCTGGCACACCTGGACA |
| | | GAGAGTGACCATCAGCTGTAGCGGCAGCAGCTCCAACATCGGCAGC |
| | | AACACCGTGAACTGGTATCAGCAGCTGCCTGGCACAGCCCCTAAACT |
| | | GCTGATCTACTACGACGACCTGCGGCCTAGCGGCGTGCCAGATAGAT |
| | | TTTCTGGCAGCAAGAGCGGCACCTCTGCCAGCCTGGCTATTTCTGGA |
| | | CTGCAGAGCGAGGACGAGGCCGACTACTATTGTCACGCCTGGGACGA |
| | | CAGCCTGAACGACTACCCTGTTTTTGGCGGAGGCACCAAGCTGACCG |
| | | TTCTAGGCCAGCCTAAAGCCGCCCCTAGCGTGACCCTGTTCCCTCCAA |
| | | GCAGCGAGGAACTGCAGGCCAACAAGGCCACCCTCGTGTGCCTGATC |
| | | AGCGACTTCTATCCTGGCGCCGTGACCGTGGCCTGGAAGGCCGATAG |
| | | CTCTCCTGTGAAGGCCGGCGTGGAAACCACCACCCCTAGCAAGCAGA |
| | | GCAACAACAAATACGCCGCCAGCAGCTACCTGAGCCTGACCCCCGAG |
| | | CAGTGGAAGTCCCACAGATCCTACAGCTGCCAAGTGACCCACGAGGG |
| | | CAGCACCGTGGAAAAGACAGTGGCCCCTACCGAGTGCAGC |
| 241 | PRT | EVQLLESGGGLVQPGGSLRLSCAASGFTFSSYAMSWVRQAPGKGLEWV |
| | | SAIGYGGDTYYADSVKGRFTISRDNSKNTLYLQMNSLRAEDTAVYYCA |
| | | RRDDYTSRDAFDYWGQGTLVTVSS |
| 242 | PRT | SYAMS |
| 243 | PRT | AIGYGGDTYYADSVKG |
| 244 | PRT | RDDYTSRDAFDY |
| 245 | PRT | QSVLTQPPSASGTPGQRVTISCSGSSSNIGSNTVNWYQQLPGTAPKLLIYY |
| | | DDLRPSGVPDRFSGSKSGTSASLAISGLQSEDEADYYCHAWDDSLNVYP |
| | | VFGGGTKLTVL |
| 246 | PRT | SGSSSNIGSNTVN |
| 247 | PRT | YDDLRPS |
| 248 | PRT | HAWDDSLNVYPV |
| 249 | DNA | GAAGTTCAGCTGCTGGAATCTGGCGGCGGACTGGTTCAACCTGGCGG |
| | | ATCTCTGAGACTGAGCTGTGCCGCCAGCGGCTTCACCTTTAGCAGCT |
| | | ACGCCATGAGCTGGGTCCGACAGGCTCCTGGCAAAGGCCTTGAATGG |
| | | GTGTCCGCCATCGGCTATGGCGGCGATACCTACTACGCCGACTCTGT |
| | | GAAGGGCAGATTCACCATCAGCCGGGACAACAGCAAGAACACCCTG |
| | | TACCTGCAGATGAACAGCCTGAGAGCCGAGGACACCGCCGTGTACTA |
| | | TTGCGCCAGAAGGGACGACTACACCAGCAGGGACGCCTTCGATTATT |
| | | GGGGCCAGGGCACACTGGTCACCGTTTCTTCA |
| 250 | DNA | AGCTACGCCATGAGC |
| 251 | DNA | GCCATCGGCTATGGCGGCGATACCTACTACGCCGACTCTGTGAAGGG |
| | | C |
| 252 | DNA | AGGGACGACTACACCAGCAGGGACGCCTTCGATTAT |
| 253 | DNA | CAGTCTGTTCTGACACAGCCTCCTAGCGCCTCTGGCACACCTGGACA |
| | | GAGAGTGACCATCAGCTGTAGCGGCAGCAGCTCCAACATCGGCAGC |
| | | AACACCGTGAACTGGTATCAGCAGCTGCCTGGCACAGCCCCTAAACT |
| | | GCTGATCTACTACGACGACCTGCGGCCTAGCGGCGTGCCAGATAGAT |
| | | TTTCTGGCAGCAAGAGCGGCACCTCTGCCAGCCTGGCTATTTCTGGA |
| | | CTGCAGAGCGAGGACGAGGCCGACTACTATTGTCACGCCTGGGACGA |
| | | CAGCCTGAACGTGTACCCTGTTTTTGGCGGAGGCACCAAGCTGACCG |
| | | TTCTA |
| 254 | DNA | AGCGGCAGCAGCTCCAACATCGGCAGCAACACCGTGAAC |
| 255 | DNA | TACGACGACCTGCGGCCTAGC |
| 256 | DNA | CACGCCTGGGACGACAGCCTGAACGTGTACCCTGTT |
| 257 | PRT | EVQLLESGGGLVQPGGSLRLSCAASGFTFSSYAMSWVRQAPGKGLEWV |
| | | SAIGYGGDTYYADSVKGRFTISRDNSKNTLYLQMNSLRAEDTAVYYCA |
| | | RRDDYTSRDAFDYWGQGTLVTVSSASTKGPSVFPLAPCSRSTSESTAAL |
| | | GCLVKDYFPEPVTVSWNSGALTSGVHTFPAVLQSSGLYSLSSVVTVPSSS |
| | | LGTKTYTCNVDHKPSNTKVDKRVESKYGPPCPPCPAPEFLGGPSVFLFPP |
| | | KPKDTLMISRTPEVTCVVVDVSQEDPEVQFNWYVDGVEVHNAKTKPRE |
| | | EQFNSTYRVVSVLTVLHQDWLNGKEYKCKVSNKGLPSSIEKTISKAKGQ |
| | | PREPQVYTLPPSQEEMTKNQVSLTCLVKGFYPSDIAVEWESNGQPENNY |
| | | KTTPPVLDSDGSFFLYSRLTVDKSRWQEGNVFSCSVMHEALHNHYTQK |
| | | SLSLSLGK |
| 258 | PRT | QSVLTQPPSASGTPGQRVTISCSGSSSNIGSNTVNWYQQLPGTAPKLLIYY |
| | | DDLRPSGVPDRFSGSKSGTSASLAISGLQSEDEADYYCHAWDDSLNVYP |
| | | VFGGGTKLTVLGQPKAAPSVTLFPPSSEELQANKATLVCLISDFYPGAVT |
| | | VAWKADSSPVKAGVETTTPSKQSNNKYAASSYLSLTPEQWKSHRSYSC |
| | | QVTHEGSTVEKTVAPTECS |
| 259 | DNA | GAAGTTCAGCTGCTGGAATCTGGCGGCGGACTGGTTCAACCTGGCGG |
| | | ATCTCTGAGACTGAGCTGTGCCGCCAGCGGCTTCACCTTTAGCAGCT |
| | | ACGCCATGAGCTGGGTCCGACAGGCTCCTGGCAAAGGCCTTGAATGG |
| | | GTGTCCGCCATCGGCTATGGCGGCGATACCTACTACGCCGACTCTGT |
| | | GAAGGGCAGATTCACCATCAGCCGGGACAACAGCAAGAACACCCTG |
| | | TACCTGCAGATGAACAGCCTGAGAGCCGAGGACACCGCCGTGTACTA |
| | | TTGCGCCAGAAGGGACGACTACACCAGCAGGGACGCCTTCGATTATT |
| | | GGGGCCAGGGCACACTGGTCACCGTTTCTTCAGCCAGCACCAAGGGC |
| | | CCCAGCGTGTTCCCTCTGGCCCCTTGTAGCAGAAGCACCAGCGAGTC |
| | | TACAGCCGCCCTGGGCTGCCTCGTGAAGGACTACTTTCCCGAGCCCG |
| | | TGACCGTGTCCTGGAACTCTGGCGCTCTGACAAGCGGCGTGCACACC |
| | | TTTCCAGCCGTGCTGCAGAGCAGCGGCCTGTACTCTCTGAGCAGCGT |
| | | CGTGACAGTGCCCAGCAGCAGCCTGGGCACCAAGACCTACACCTGTA |
| | | ACGTGGACCACAAGCCCAGCAACACCAAGGTGGACAAGCGGGTGGA |
| | | ATCTAAGTACGGCCCTCCCTGCCCTCCTTGCCCAGCCCCTGAATTTCT |
| | | GGGCGGACCCTCCGTGTTCCTGTTCCCCCCAAAGCCCAAGGACACCC |
| | | TGATGATCAGCCGGACCCCCGAAGTGACCTGCGTGGTGGTGGATGTG |
| | | TCCCAGGAAGATCCCGAGGTGCAGTTCAATTGGTACGTGGACGGCGT |
| | | GGAAGTGCACAACGCCAAGACCAAGCCCAGAGAGGAACAGTTCAAC |
| | | AGCACCTACCGGGTGGTGTCCGTGCTGACAGTGCTGCACCAGGACTG |
| | | GCTGAACGGCAAAGAGTACAAGTGCAAGGTGTCCAACAAGGGCCTG |
| | | CCCAGCTCCATCGAGAAAACCATCAGCAAGGCCAAGGGCCAGCCCC |
| | | GCGAACCCCAGGTGTACACACTGCCTCCAAGCCAGGAAGAGATGAC |
| | | CAAGAACCAGGTGTCCCTGACCTGTCTCGTGAAAGGCTTCTACCCCT |
| | | CCGATATCGCCGTGGAATGGGAGAGCAACGGCCAGCCCGAGAACAA |
| | | CTACAAGACCACCCCCCCTGTGCTGGACAGCGACGGCTCATTCTTCCT |
| | | GTACAGCAGACTGACCGTGGACAAGAGCCGGTGGCAGGAAGGCAAC |
| | | GTGTTCAGCTGCAGCGTGATGCACGAGGCCCTGCACAACCACTACAC |
| | | CCAGAAGTCCCTGTCTCTGAGCCTGGGCAAG |
| 260 | DNA | CAGTCTGTTCTGACACAGCCTCCTAGCGCCTCTGGCACACCTGGACA |
| | | GAGAGTGACCATCAGCTGTAGCGGCAGCAGCTCCAACATCGGCAGC |
| | | AACACCGTGAACTGGTATCAGCAGCTGCCTGGCACAGCCCCTAAACT |
| | | GCTGATCTACTACGACGACCTGCGGCCTAGCGGCGTGCCAGATAGAT |
| | | TTTCTGGCAGCAAGAGCGGCACCTCTGCCAGCCTGGCTATTTCTGGA |
| | | CTGCAGAGCGAGGACGAGGCCGACTACTATTGTCACGCCTGGGACGA |
| | | CAGCCTGAACGTGTACCCTGTTTTTGGCGGAGGCACCAAGCTGACCG |
| | | TTCTAGGCCAGCCTAAAGCCGCCCCTAGCGTGACCCTGTTCCCTCCAA |
| | | GCAGCGAGGAACTGCAGGCCAACAAGGCCACCCTCGTGTGCCTGATC |
| | | AGCGACTTCTATCCTGGCGCCGTGACCGTGGCCTGGAAGGCCGATAG |
| | | CTCTCCTGTGAAGGCCGGCGTGGAAACCACCACCCCTAGCAAGCAGA |
| | | GCAACAACAAATACGCCGCCAGCAGCTACCTGAGCCTGACCCCCGAG |
| | | CAGTGGAAGTCCCACAGATCCTACAGCTGCCAAGTGACCCACGAGGG |
| | | CAGCACCGTGGAAAAGACAGTGGCCCCTACCGAGTGCAGC |
| 261 | PRT | EVQLLESGGGLVQPGGSLRLSCAASGFTFSSYAMSWVRQAPGKGLEWV |
| | | SAIGYGGDTYYADSVKGRFTISRDNSKNTLYLQMNSLRAEDTAVYYCA |
| | | RRDDYTSRDAFDYWGQGTLVTVSS |
| 262 | PRT | SYAMS |
| 263 | PRT | AIGYGGDTYYADSVKG |
| 264 | PRT | RDDYTSRDAFDY |
| 265 | PRT | QSVLTQPPSASGTPGQRVTISCSGSSSNIGSNTVNWYQQLPGTAPKLLIYY |
| | | DDLRPSGVPDRFSGSKSGTSASLAISGLQSEDEADYYCHAWDDSLNVIPV |
| | | FGGGTKLTVL |
| 266 | PRT | SGSSSNIGSNTVN |
| 267 | PRT | YDDLRPS |
| 268 | PRT | HAWDDSLNVIPV |
| 269 | DNA | GAAGTTCAGCTGCTGGAATCTGGCGGCGGACTGGTTCAACCTGGCGG |
| | | ATCTCTGAGACTGAGCTGTGCCGCCAGCGGCTTCACCTTTAGCAGCT |
| | | ACGCCATGAGCTGGGTCCGACAGGCTCCTGGCAAAGGCCTTGAATGG |
| | | GTGTCCGCCATCGGCTATGGCGGCGATACCTACTACGCCGACTCTGT |
| | | GAAGGGCAGATTCACCATCAGCCGGGACAACAGCAAGAACACCCTG |
| | | TACCTGCAGATGAACAGCCTGAGAGCCGAGGACACCGCCGTGTACTA |
| | | TTGCGCCAGAAGGGACGACTACACCAGCAGGGACGCCTTCGATTATT |
| | | GGGGCCAGGGCACACTGGTCACCGTTTCTTCA |
| 270 | DNA | AGCTACGCCATGAGC |
| 271 | DNA | GCCATCGGCTATGGCGGCGATACCTACTACGCCGACTCTGTGAAGGG |
| | | C |
| 272 | DNA | AGGGACGACTACACCAGCAGGGACGCCTTCGATTAT |
| 273 | DNA | CAGTCTGTTCTGACACAGCCTCCTAGCGCCTCTGGCACACCTGGACA |
| | | GAGAGTGACCATCAGCTGTAGCGGCAGCAGCTCCAACATCGGCAGC |
| | | AACACCGTGAACTGGTATCAGCAGCTGCCTGGCACAGCCCCTAAACT |
| | | GCTGATCTACTACGACGACCTGCGGCCTAGCGGCGTGCCAGATAGAT |
| | | TTTCTGGCAGCAAGAGCGGCACCTCTGCCAGCCTGGCTATTTCTGGA |
| | | CTGCAGAGCGAGGACGAGGCCGACTACTATTGTCACGCCTGGGACGA |
| | | CAGCCTGAACGTGATCCCTGTTTTTGGCGGAGGCACCAAGCTGACCG |
| | | TTCTA |
| 274 | DNA | AGCGGCAGCAGCTCCAACATCGGCAGCAACACCGTGAAC |
| 275 | DNA | TACGACGACCTGCGGCCTAGC |
| 276 | DNA | CACGCCTGGGACGACAGCCTGAACGTGATCCCTGTT |
| 277 | PRT | EVQLLESGGGLVQPGGSLRLSCAASGFTFSSYAMSWVRQAPGKGLEWV |
| | | SAIGYGGDTYYADSVKGRFTISRDNSKNTLYLQMNSLRAEDTAVYYCA |
| | | RRDDYTSRDAFDYWGQGTLVTVSSASTKGPSVFPLAPCSRSTSESTAAL |
| | | GCLVKDYFPEPVTVSWNSGALTSGVHTFPAVLQSSGLYSLSSVVTVPSSS |
| | | LGTKTYTCNVDHKPSNTKVDKRVESKYGPPCPPCPAPEFLGGPSVFLFPP |
| | | KPKDTLMISRTPEVTCVVVDVSQEDPEVQFNWYVDGVEVHNAKTKPRE |
| | | EQFNSTYRVVSVLTVLHQDWLNGKEYKCKVSNKGLPSSIEKTISKAKGQ |
| | | PREPQVYTLPPSQEEMTKNQVSLTCLVKGFYPSDIAVEWESNGQPENNY |
| | | KTTPPVLDSDGSFFLYSRLTVDKSRWQEGNVFSCSVMHEALHNHYTQK |
| | | SLSLSLGK |
| 278 | PRT | QSVLTQPPSASGTPGQRVTISCSGSSSNIGSNTVNWYQQLPGTAPKLLIYY |
| | | DDLRPSGVPDRFSGSKSGTSASLAISGLQSEDEADYYCHAWDDSLNVIPV |
| | | FGGGTKLTVLGQPKAAPSVTLFPPSSEELQANKATLVCLISDFYPGAVTV |
| | | AWKADSSPVKAGVETTTPSKQSNNKYAASSYLSLTPEQWKSHRSYSCQ |
| | | VTHEGSTVEKTVAPTECS |
| 279 | DNA | GAAGTTCAGCTGCTGGAATCTGGCGGCGGACTGGTTCAACCTGGCGG |
| | | ATCTCTGAGACTGAGCTGTGCCGCCAGCGGCTTCACCTTTAGCAGCT |
| | | ACGCCATGAGCTGGGTCCGACAGGCTCCTGGCAAAGGCCTTGAATGG |
| | | GTGTCCGCCATCGGCTATGGCGGCGATACCTACTACGCCGACTCTGT |
| | | GAAGGGCAGATTCACCATCAGCCGGGACAACAGCAAGAACACCCTG |
| | | TACCTGCAGATGAACAGCCTGAGAGCCGAGGACACCGCCGTGTACTA |
| | | TTGCGCCAGAAGGGACGACTACACCAGCAGGGACGCCTTCGATTATT |
| | | GGGGCCAGGGCACACTGGTCACCGTTTCTTCAGCCAGCACCAAGGGC |
| | | CCCAGCGTGTTCCCTCTGGCCCCTTGTAGCAGAAGCACCAGCGAGTC |
| | | TACAGCCGCCCTGGGCTGCCTCGTGAAGGACTACTTTCCCGAGCCCG |
| | | TGACCGTGTCCTGGAACTCTGGCGCTCTGACAAGCGGCGTGCACACC |
| | | TTTCCAGCCGTGCTGCAGAGCAGCGGCCTGTACTCTCTGAGCAGCGT |
| | | CGTGACAGTGCCCAGCAGCAGCCTGGGCACCAAGACCTACACCTGTA |
| | | ACGTGGACCACAAGCCCAGCAACACCAAGGTGGACAAGCGGGTGGA |
| | | ATCTAAGTACGGCCCTCCCTGCCCTCCTTGCCCAGCCCCTGAATTTCT |
| | | GGGCGGACCCTCCGTGTTCCTGTTCCCCCCAAAGCCCAAGGACACCC |
| | | TGATGATCAGCCGGACCCCCGAAGTGACCTGCGTGGTGGTGGATGTG |
| | | TCCCAGGAAGATCCCGAGGTGCAGTTCAATTGGTACGTGGACGGCGT |
| | | GGAAGTGCACAACGCCAAGACCAAGCCCAGAGAGGAACAGTTCAAC |
| | | AGCACCTACCGGGTGGTGTCCGTGCTGACAGTGCTGCACCAGGACTG |
| | | GCTGAACGGCAAAGAGTACAAGTGCAAGGTGTCCAACAAGGGCCTG |
| | | CCCAGCTCCATCGAGAAAACCATCAGCAAGGCCAAGGGCCAGCCCC |
| | | GCGAACCCCAGGTGTACACACTGCCTCCAAGCCAGGAAGAGATGAC |
| | | CAAGAACCAGGTGTCCCTGACCTGTCTCGTGAAAGGCTTCTACCCCT |
| | | CCGATATCGCCGTGGAATGGGAGAGCAACGGCCAGCCCGAGAACAA |
| | | CTACAAGACCACCCCCCCTGTGCTGGACAGCGACGGCTCATTCTTCCT |
| | | GTACAGCAGACTGACCGTGGACAAGAGCCGGTGGCAGGAAGGCAAC |
| | | GTGTTCAGCTGCAGCGTGATGCACGAGGCCCTGCACAACCACTACAC |
| | | CCAGAAGTCCCTGTCTCTGAGCCTGGGCAAG |
| 280 | DNA | CAGTCTGTTCTGACACAGCCTCCTAGCGCCTCTGGCACACCTGGACA |
| | | GAGAGTGACCATCAGCTGTAGCGGCAGCAGCTCCAACATCGGCAGC |
| | | AACACCGTGAACTGGTATCAGCAGCTGCCTGGCACAGCCCCTAAACT |
| | | GCTGATCTACTACGACGACCTGCGGCCTAGCGGCGTGCCAGATAGAT |
| | | TTTCTGGCAGCAAGAGCGGCACCTCTGCCAGCCTGGCTATTTCTGGA |
| | | CTGCAGAGCGAGGACGAGGCCGACTACTATTGTCACGCCTGGGACGA |
| | | CAGCCTGAACGTGATCCCTGTTTTTGGCGGAGGCACCAAGCTGACCG |
| | | TTCTAGGCCAGCCTAAAGCCGCCCCTAGCGTGACCCTGTTCCCTCCAA |
| | | GCAGCGAGGAACTGCAGGCCAACAAGGCCACCCTCGTGTGCCTGATC |
| | | AGCGACTTCTATCCTGGCGCCGTGACCGTGGCCTGGAAGGCCGATAG |
| | | CTCTCCTGTGAAGGCCGGCGTGGAAACCACCACCCCTAGCAAGCAGA |
| | | GCAACAACAAATACGCCGCCAGCAGCTACCTGAGCCTGACCCCCGAG |
| | | CAGTGGAAGTCCCACAGATCCTACAGCTGCCAAGTGACCCACGAGGG |
| | | CAGCACCGTGGAAAAGACAGTGGCCCCTACCGAGTGCAGC |
| 281 | PRT | EVQLLESGGGLVQPGGSLRLSCAASGFTFYSYAMLWVRQAPGKGLEWV |
| | | SAIGTGGDTYYADSVKGRFTISRDNSKNTLYLQMNSLRAEDTAVYYCA |
| | | RRDDYTSRDAFDYWGQGTLVTVSS |
| 282 | PRT | SYAML |
| 283 | PRT | AIGTGGDTYYADSVKG |
| 284 | PRT | RDDYTSRDAFDY |
| 285 | PRT | QSVLTQPPSASGTPGQRVTISCSGSSSNIGSNTVNWYQQLPGTAPKLLIYY |
| | | DDLRPSGVPDRFSGSKSGTSASLAISGLQSEDEADYYCAAWDDSLNDYV |
| | | VFGGGTKLTVL |
| 286 | PRT | SGSSSNIGSNTVN |
| 287 | PRT | YDDLRPS |
| 288 | PRT | AAWDDSLNDYVV |
| 289 | DNA | GAAGTTCAGCTGCTGGAATCTGGCGGCGGACTGGTTCAACCTGGCGG |
| | | ATCTCTGAGACTGAGCTGTGCCGCCAGCGGCTTCACCTTTTACAGCTA |
| | | CGCCATGCTGTGGGTCCGACAGGCCCCTGGAAAAGGCCTTGAATGGG |
| | | TGTCCGCCATCGGCACAGGCGGCGATACCTACTATGCCGACTCTGTG |
| | | AAGGGCAGATTCACCATCAGCCGGGACAACAGCAAGAACACCCTGT |
| | | ACCTGCAGATGAACAGCCTGAGAGCCGAGGACACCGCCGTGTACTAT |
| | | TGCGCCAGAAGGGACGACTACACCAGCAGGGACGCCTTCGATTATTG |
| | | GGGCCAGGGCACACTGGTCACCGTTTCTTCA |
| 290 | DNA | AGCTACGCCATGCTG |
| 291 | DNA | GCCATCGGCACAGGCGGCGATACCTACTATGCCGACTCTGTGAAGGG |
| | | C |
| 292 | DNA | AGGGACGACTACACCAGCAGGGACGCCTTCGATTAT |
| 293 | DNA | CAGTCTGTTCTGACACAGCCTCCTAGCGCCTCTGGCACACCTGGACA |
| | | GAGAGTGACCATCAGCTGTAGCGGCAGCAGCTCCAACATCGGCAGC |
| | | AACACCGTGAACTGGTATCAGCAGCTGCCTGGCACAGCCCCTAAACT |
| | | GCTGATCTACTACGACGACCTGCGGCCTAGCGGCGTGCCAGATAGAT |
| | | TTTCTGGCAGCAAGAGCGGCACCTCTGCCAGCCTGGCTATTTCTGGA |
| | | CTGCAGAGCGAGGACGAGGCCGACTATTATTGTGCCGCCTGGGACGA |
| | | CAGCCTGAACGACTACGTTGTGTTTGGCGGAGGCACCAAGCTGACCG |
| | | TTCTA |
| 294 | DNA | AGCGGCAGCAGCTCCAACATCGGCAGCAACACCGTGAAC |
| 295 | DNA | TACGACGACCTGCGGCCTAGC |
| 296 | DNA | GCCGCCTGGGACGACAGCCTGAACGACTACGTTGTG |
| 297 | PRT | EVQLLESGGGLVQPGGSLRLSCAASGFTFYSYAMLWVRQAPGKGLEWV |
| | | SAIGTGGDTYYADSVKGRFTISRDNSKNTLYLQMNSLRAEDTAVYYCA |
| | | RRDDYTSRDAFDYWGQGTLVTVSSASTKGPSVFPLAPCSRSTSESTAAL |
| | | GCLVKDYFPEPVTVSWNSGALTSGVHTFPAVLQSSGLYSLSSVVTVPSSS |
| | | LGTKTYTCNVDHKPSNTKVDKRVESKYGPPCPPCPAPEFLGGPSVFLFPP |
| | | KPKDTLMISRTPEVTCVVVDVSQEDPEVQFNWYVDGVEVHNAKTKPRE |
| | | EQFNSTYRVVSVLTVLHQDWLNGKEYKCKVSNKGLPSSIEKTISKAKGQ |
| | | PREPQVYTLPPSQEEMTKNQVSLTCLVKGFYPSDIAVEWESNGQPENNY |
| | | KTTPPVLDSDGSFFLYSRLTVDKSRWQEGNVFSCSVMHEALHNHYTQK |
| | | SLSLSLGK |
| 298 | PRT | QSVLTQPPSASGTPGQRVTISCSGSSSNIGSNTVNWYQQLPGTAPKLLIYY |
| | | DDLRPSGVPDRFSGSKSGTSASLAISGLQSEDEADYYCAAWDDSLNDYV |
| | | VFGGGTKLTVLGQPKAAPSVTLFPPSSEELQANKATLVCLISDFYPGAVT |
| | | VAWKADSSPVKAGVETTTPSKQSNNKYAASSYLSLTPEQWKSHRSYSC |
| | | QVTHEGSTVEKTVAPTECS |
| 299 | DNA | GAAGTTCAGCTGCTGGAATCTGGCGGCGGACTGGTTCAACCTGGCGG |
| | | ATCTCTGAGACTGAGCTGTGCCGCCAGCGGCTTCACCTTTTACAGCTA |
| | | CGCCATGCTGTGGGTCCGACAGGCCCCTGGAAAAGGCCTTGAATGGG |
| | | TGTCCGCCATCGGCACAGGCGGCGATACCTACTATGCCGACTCTGTG |
| | | AAGGGCAGATTCACCATCAGCCGGGACAACAGCAAGAACACCCTGT |
| | | ACCTGCAGATGAACAGCCTGAGAGCCGAGGACACCGCCGTGTACTAT |
| | | TGCGCCAGAAGGGACGACTACACCAGCAGGGACGCCTTCGATTATTG |
| | | GGGCCAGGGCACACTGGTCACCGTTTCTTCAGCCAGCACCAAGGGCC |
| | | CCAGCGTGTTCCCTCTGGCCCCTTGTAGCAGAAGCACCAGCGAGTCT |
| | | ACAGCCGCCCTGGGCTGCCTCGTGAAGGACTACTTTCCCGAGCCCGT |
| | | GACCGTGTCCTGGAACTCTGGCGCTCTGACAAGCGGCGTGCACACCT |
| | | TTCCAGCCGTGCTGCAGAGCAGCGGCCTGTACTCTCTGAGCAGCGTC |
| | | GTGACAGTGCCCAGCAGCAGCCTGGGCACCAAGACCTACACCTGTAA |
| | | CGTGGACCACAAGCCCAGCAACACCAAGGTGGACAAGCGGGTGGAA |
| | | TCTAAGTACGGCCCTCCCTGCCCTCCTTGCCCAGCCCCTGAATTTCTG |
| | | GGCGGACCCTCCGTGTTCCTGTTCCCCCCAAAGCCCAAGGACACCCT |
| | | GATGATCAGCCGGACCCCCGAAGTGACCTGCGTGGTGGTGGATGTGT |
| | | CCCAGGAAGATCCCGAGGTGCAGTTCAATTGGTACGTGGACGGCGTG |
| | | GAAGTGCACAACGCCAAGACCAAGCCCAGAGAGGAACAGTTCAACA |
| | | GCACCTACCGGGTGGTGTCCGTGCTGACAGTGCTGCACCAGGACTGG |
| | | CTGAACGGCAAAGAGTACAAGTGCAAGGTGTCCAACAAGGGCCTGC |
| | | CCAGCTCCATCGAGAAAACCATCAGCAAGGCCAAGGGCCAGCCCCG |
| | | CGAACCCCAGGTGTACACACTGCCTCCAAGCCAGGAAGAGATGACC |
| | | AAGAACCAGGTGTCCCTGACCTGTCTCGTGAAAGGCTTCTACCCCTC |
| | | CGATATCGCCGTGGAATGGGAGAGCAACGGCCAGCCCGAGAACAAC |
| | | TACAAGACCACCCCCCCTGTGCTGGACAGCGACGGCTCATTCTTCCT |
| | | GTACAGCAGACTGACCGTGGACAAGAGCCGGTGGCAGGAAGGCAAC |
| | | GTGTTCAGCTGCAGCGTGATGCACGAGGCCCTGCACAACCACTACAC |
| | | CCAGAAGTCCCTGTCTCTGAGCCTGGGCAAG |
| 300 | DNA | CAGTCTGTTCTGACACAGCCTCCTAGCGCCTCTGGCACACCTGGACA |
| | | GAGAGTGACCATCAGCTGTAGCGGCAGCAGCTCCAACATCGGCAGC |
| | | AACACCGTGAACTGGTATCAGCAGCTGCCTGGCACAGCCCCTAAACT |
| | | GCTGATCTACTACGACGACCTGCGGCCTAGCGGCGTGCCAGATAGAT |
| | | TTTCTGGCAGCAAGAGCGGCACCTCTGCCAGCCTGGCTATTTCTGGA |
| | | CTGCAGAGCGAGGACGAGGCCGACTATTATTGTGCCGCCTGGGACGA |
| | | CAGCCTGAACGACTACGTTGTGTTTGGCGGAGGCACCAAGCTGACCG |
| | | TTCTAGGCCAGCCTAAAGCCGCCCCTAGCGTGACCCTGTTCCCTCCAA |
| | | GCAGCGAGGAACTGCAGGCCAACAAGGCCACCCTCGTGTGCCTGATC |
| | | AGCGACTTCTATCCTGGCGCCGTGACCGTGGCCTGGAAGGCCGATAG |
| | | CTCTCCTGTGAAGGCCGGCGTGGAAACCACCACCCCTAGCAAGCAGA |
| | | GCAACAACAAATACGCCGCCAGCAGCTACCTGAGCCTGACCCCCGAG |
| | | CAGTGGAAGTCCCACAGATCCTACAGCTGCCAAGTGACCCACGAGGG |
| | | CAGCACCGTGGAAAAGACAGTGGCCCCTACCGAGTGCAGC |
| 301 | PRT | EVQLLESGGGLVQPGGSLRLSCAASGFTFYSYAMLWVRQAPGKGLEWV |
| | | SAIGTGGDTYYADSVKGRFTISRDNSKNTLYLQMNSLRAEDTAVYYCA |
| | | RRDDYTSRDAFDYWGQGTLVTVSS |
| 302 | PRT | SYAML |
| 303 | PRT | AIGTGGDTYYADSVKG |
| 304 | PRT | RDDYTSRDAFDY |
| 305 | PRT | QSVLTQPPSASGTPGQRVTISCSGSSSNIGSNTVNWYQQLPGTAPKLLIYY |
| | | DDLRPSGVPDRFSGSKSGTSASLAISGLQSEDEADYYCAAWDDSLNVYV |
| | | VFGGGTKLTVL |
| 306 | PRT | SGSSSNIGSNTVN |
| 307 | PRT | YDDLRPS |
| 308 | PRT | AAWDDSLNVYVV |
| 309 | DNA | GAAGTTCAGCTGCTGGAATCTGGCGGCGGACTGGTTCAACCTGGCGG |
| | | ATCTCTGAGACTGAGCTGTGCCGCCAGCGGCTTCACCTTTTACAGCTA |
| | | CGCCATGCTGTGGGTCCGACAGGCCCCTGGAAAAGGCCTTGAATGGG |
| | | TGTCCGCCATCGGCACAGGCGGCGATACCTACTATGCCGACTCTGTG |
| | | AAGGGCAGATTCACCATCAGCCGGGACAACAGCAAGAACACCCTGT |
| | | ACCTGCAGATGAACAGCCTGAGAGCCGAGGACACCGCCGTGTACTAT |
| | | TGCGCCAGAAGGGACGACTACACCAGCAGGGACGCCTTCGATTATTG |
| | | GGGCCAGGGCACACTGGTCACCGTTTCTTCA |
| 310 | DNA | AGCTACGCCATGCTG |
| 311 | DNA | GCCATCGGCACAGGCGGCGATACCTACTATGCCGACTCTGTGAAGGG |
| | | C |
| 312 | DNA | AGGGACGACTACACCAGCAGGGACGCCTTCGATTAT |
| 313 | DNA | CAGTCTGTTCTGACACAGCCTCCTAGCGCCTCTGGCACACCTGGACA |
| | | GAGAGTGACCATCAGCTGTAGCGGCAGCAGCTCCAACATCGGCAGC |
| | | AACACCGTGAACTGGTATCAGCAGCTGCCTGGCACAGCCCCTAAACT |
| | | GCTGATCTACTACGACGACCTGCGGCCTAGCGGCGTGCCAGATAGAT |
| | | TTTCTGGCAGCAAGAGCGGCACCTCTGCCAGCCTGGCTATTTCTGGA |
| | | CTGCAGAGCGAGGACGAGGCCGACTATTATTGTGCCGCCTGGGACGA |
| | | CAGCCTGAACGTGTACGTTGTGTTTGGCGGAGGCACCAAGCTGACCG |
| | | TTCTA |
| 314 | DNA | AGCGGCAGCAGCTCCAACATCGGCAGCAACACCGTGAAC |
| 315 | DNA | TACGACGACCTGCGGCCTAGC |
| 316 | DNA | GCCGCCTGGGACGACAGCCTGAACGTGTACGTTGTG |
| 317 | PRT | EVQLLESGGGLVQPGGSLRLSCAASGFTFYSYAMLWVRQAPGKGLEWV |
| | | SAIGTGGDTYYADSVKGRFTISRDNSKNTLYLQMNSLRAEDTAVYYCA |
| | | RRDDYTSRDAFDYWGQGTLVTVSSASTKGPSVFPLAPCSRSTSESTAAL |
| | | GCLVKDYFPEPVTVSWNSGALTSGVHTFPAVLQSSGLYSLSSVVTVPSSS |
| | | LGTKTYTCNVDHKPSNTKVDKRVESKYGPPCPPCPAPEFLGGPSVFLFPP |
| | | KPKDTLMISRTPEVTCVVVDVSQEDPEVQFNWYVDGVEVHNAKTKPRE |
| | | EQFNSTYRVVSVLTVLHQDWLNGKEYKCKVSNKGLPSSIEKTISKAKGQ |
| | | PREPQVYTLPPSQEEMTKNQVSLTCLVKGFYPSDIAVEWESNGQPENNY |
| | | KTTPPVLDSDGSFFLYSRLTVDKSRWQEGNVFSCSVMHEALHNHYTQK |
| | | SLSLSLGK |
| 318 | PRT | QSVLTQPPSASGTPGQRVTISCSGSSSNIGSNTVNWYQQLPGTAPKLLIYY |
| | | DDLRPSGVPDRFSGSKSGTSASLAISGLQSEDEADYYCAAWDDSLNVYV |
| | | VFGGGTKLTVLGQPKAAPSVTLFPPSSEELQANKATLVCLISDFYPGAVT |
| | | VAWKADSSPVKAGVETTTPSKQSNNKYAASSYLSLTPEQWKSHRSYSC |
| | | QVTHEGSTVEKTVAPTECS |
| 319 | DNA | GAAGTTCAGCTGCTGGAATCTGGCGGCGGACTGGTTCAACCTGGCGG |
| | | ATCTCTGAGACTGAGCTGTGCCGCCAGCGGCTTCACCTTTTACAGCTA |
| | | CGCCATGCTGTGGGTCCGACAGGCCCCTGGAAAAGGCCTTGAATGGG |
| | | TGTCCGCCATCGGCACAGGCGGCGATACCTACTATGCCGACTCTGTG |
| | | AAGGGCAGATTCACCATCAGCCGGGACAACAGCAAGAACACCCTGT |
| | | ACCTGCAGATGAACAGCCTGAGAGCCGAGGACACCGCCGTGTACTAT |
| | | TGCGCCAGAAGGGACGACTACACCAGCAGGGACGCCTTCGATTATTG |
| | | GGGCCAGGGCACACTGGTCACCGTTTCTTCAGCCAGCACCAAGGGCC |
| | | CCAGCGTGTTCCCTCTGGCCCCTTGTAGCAGAAGCACCAGCGAGTCT |
| | | ACAGCCGCCCTGGGCTGCCTCGTGAAGGACTACTTTCCCGAGCCCGT |
| | | GACCGTGTCCTGGAACTCTGGCGCTCTGACAAGCGGCGTGCACACCT |
| | | TTCCAGCCGTGCTGCAGAGCAGCGGCCTGTACTCTCTGAGCAGCGTC |
| | | GTGACAGTGCCCAGCAGCAGCCTGGGCACCAAGACCTACACCTGTAA |
| | | CGTGGACCACAAGCCCAGCAACACCAAGGTGGACAAGCGGGTGGAA |
| | | TCTAAGTACGGCCCTCCCTGCCCTCCTTGCCCAGCCCCTGAATTTCTG |
| | | GGCGGACCCTCCGTGTTCCTGTTCCCCCCAAAGCCCAAGGACACCCT |
| | | GATGATCAGCCGGACCCCCGAAGTGACCTGCGTGGTGGTGGATGTGT |
| | | CCCAGGAAGATCCCGAGGTGCAGTTCAATTGGTACGTGGACGGCGTG |
| | | GAAGTGCACAACGCCAAGACCAAGCCCAGAGAGGAACAGTTCAACA |
| | | GCACCTACCGGGTGGTGTCCGTGCTGACAGTGCTGCACCAGGACTGG |
| | | CTGAACGGCAAAGAGTACAAGTGCAAGGTGTCCAACAAGGGCCTGC |
| | | CCAGCTCCATCGAGAAAACCATCAGCAAGGCCAAGGGCCAGCCCCG |
| | | CGAACCCCAGGTGTACACACTGCCTCCAAGCCAGGAAGAGATGACC |
| | | AAGAACCAGGTGTCCCTGACCTGTCTCGTGAAAGGCTTCTACCCCTC |
| | | CGATATCGCCGTGGAATGGGAGAGCAACGGCCAGCCCGAGAACAAC |
| | | TACAAGACCACCCCCCCTGTGCTGGACAGCGACGGCTCATTCTTCCT |
| | | GTACAGCAGACTGACCGTGGACAAGAGCCGGTGGCAGGAAGGCAAC |
| | | GTGTTCAGCTGCAGCGTGATGCACGAGGCCCTGCACAACCACTACAC |
| | | CCAGAAGTCCCTGTCTCTGAGCCTGGGCAAG |
| 320 | DNA | CAGTCTGTTCTGACACAGCCTCCTAGCGCCTCTGGCACACCTGGACA |
| | | GAGAGTGACCATCAGCTGTAGCGGCAGCAGCTCCAACATCGGCAGC |
| | | AACACCGTGAACTGGTATCAGCAGCTGCCTGGCACAGCCCCTAAACT |
| | | GCTGATCTACTACGACGACCTGCGGCCTAGCGGCGTGCCAGATAGAT |
| | | TTTCTGGCAGCAAGAGCGGCACCTCTGCCAGCCTGGCTATTTCTGGA |
| | | CTGCAGAGCGAGGACGAGGCCGACTATTATTGTGCCGCCTGGGACGA |
| | | CAGCCTGAACGTGTACGTTGTGTTTGGCGGAGGCACCAAGCTGACCG |
| | | TTCTAGGCCAGCCTAAAGCCGCCCCTAGCGTGACCCTGTTCCCTCCAA |
| | | GCAGCGAGGAACTGCAGGCCAACAAGGCCACCCTCGTGTGCCTGATC |
| | | AGCGACTTCTATCCTGGCGCCGTGACCGTGGCCTGGAAGGCCGATAG |
| | | CTCTCCTGTGAAGGCCGGCGTGGAAACCACCACCCCTAGCAAGCAGA |
| | | GCAACAACAAATACGCCGCCAGCAGCTACCTGAGCCTGACCCCCGAG |
| | | CAGTGGAAGTCCCACAGATCCTACAGCTGCCAAGTGACCCACGAGGG |
| | | CAGCACCGTGGAAAAGACAGTGGCCCCTACCGAGTGCAGC |
| 321 | PRT | EVQLLESGGGLVQPGGSLRLSCAASGFTFYSYAMSWVRQAPGKGLEWV |
| | | SAIGYGGDTYYADSVKGRFTISRDNSKNTLYLQMNSLRAEDTAVYYCA |
| | | RRDDYTSRDAFDYWGQGTLVTVSS |
| 322 | PRT | SYAMS |
| 323 | PRT | AIGYGGDTYYADSVKG |
| 324 | PRT | RDDYTSRDAFDY |
| 325 | PRT | QSVLTQPPSASGTPGQRVTISCSGSSSNIGSNTVNWYQQLPGTAPKLLIYY |
| | | DDLRPSGVPDRFSGSKSGTSASLAISGLQSEDEADYYCHAWDDSLNVYP |
| | | VFGGGTKLTVL |
| 326 | PRT | SGSSSNIGSNTVN |
| 327 | PRT | YDDLRPS |
| 328 | PRT | HAWDDSLNVYPV |
| 329 | DNA | GAAGTTCAGCTGCTGGAATCTGGCGGCGGACTGGTTCAACCTGGCGG |
| | | ATCTCTGAGACTGAGCTGTGCCGCCAGCGGCTTCACCTTTTACAGCTA |
| | | CGCCATGAGCTGGGTCCGACAGGCCCCTGGAAAAGGCCTTGAATGGG |
| | | TGTCCGCCATCGGCTATGGCGGCGATACCTACTACGCCGACTCTGTG |
| | | AAGGGCAGATTCACCATCAGCCGGGACAACAGCAAGAACACCCTGT |
| | | ACCTGCAGATGAACAGCCTGAGAGCCGAGGACACCGCCGTGTACTAT |
| | | TGCGCCAGAAGGGACGACTACACCAGCAGGGACGCCTTCGATTATTG |
| | | GGGCCAGGGCACACTGGTCACCGTTTCTTCA |
| 330 | DNA | AGCTACGCCATGAGC |
| 331 | DNA | GCCATCGGCTATGGCGGCGATACCTACTACGCCGACTCTGTGAAGGG |
| | | C |
| 332 | DNA | AGGGACGACTACACCAGCAGGGACGCCTTCGATTAT |
| 333 | DNA | CAGTCTGTTCTGACACAGCCTCCTAGCGCCTCTGGCACACCTGGACA |
| | | GAGAGTGACCATCAGCTGTAGCGGCAGCAGCTCCAACATCGGCAGC |
| | | AACACCGTGAACTGGTATCAGCAGCTGCCTGGCACAGCCCCTAAACT |
| | | GCTGATCTACTACGACGACCTGCGGCCTAGCGGCGTGCCAGATAGAT |
| | | TTTCTGGCAGCAAGAGCGGCACCTCTGCCAGCCTGGCTATTTCTGGA |
| | | CTGCAGAGCGAGGACGAGGCCGACTACTATTGTCACGCCTGGGACGA |
| | | CAGCCTGAACGTGTACCCTGTTTTTGGCGGAGGCACCAAGCTGACCG |
| | | TTCTA |
| 334 | DNA | AGCGGCAGCAGCTCCAACATCGGCAGCAACACCGTGAAC |
| 335 | DNA | TACGACGACCTGCGGCCTAGC |
| 336 | DNA | CACGCCTGGGACGACAGCCTGAACGTGTACCCTGTT |
| 337 | PRT | EVQLLESGGGLVQPGGSLRLSCAASGFTFYSYAMSWVRQAPGKGLEWV |
| | | SAIGYGGDTYYADSVKGRFTISRDNSKNTLYLQMNSLRAEDTAVYYCA |
| | | RRDDYTSRDAFDYWGQGTLVTVSSASTKGPSVFPLAPCSRSTSESTAAL |
| | | GCLVKDYFPEPVTVSWNSGALTSGVHTFPAVLQSSGLYSLSSVVTVPSSS |
| | | LGTKTYTCNVDHKPSNTKVDKRVESKYGPPCPPCPAPEFLGGPSVFLFPP |
| | | KPKDTLMISRTPEVTCVVVDVSQEDPEVQFNWYVDGVEVHNAKTKPRE |
| | | EQFNSTYRVVSVLTVLHQDWLNGKEYKCKVSNKGLPSSIEKTISKAKGQ |
| | | PREPQVYTLPPSQEEMTKNQVSLTCLVKGFYPSDIAVEWESNGQPENNY |
| | | KTTPPVLDSDGSFFLYSRLTVDKSRWQEGNVFSCSVMHEALHNHYTQK |
| | | SLSLSLGK |
| 338 | PRT | QSVLTQPPSASGTPGQRVTISCSGSSSNIGSNTVNWYQQLPGTAPKLLIYY |
| | | DDLRPSGVPDRFSGSKSGTSASLAISGLQSEDEADYYCHAWDDSLNVYP |
| | | VFGGGTKLTVLGQPKAAPSVTLFPPSSEELQANKATLVCLISDFYPGAVT |
| | | VAWKADSSPVKAGVETTTPSKQSNNKYAASSYLSLTPEQWKSHRSYSC |
| | | QVTHEGSTVEKTVAPTECS |
| 339 | DNA | GAAGTTCAGCTGCTGGAATCTGGCGGCGGACTGGTTCAACCTGGCGG |
| | | ATCTCTGAGACTGAGCTGTGCCGCCAGCGGCTTCACCTTTTACAGCTA |
| | | CGCCATGAGCTGGGTCCGACAGGCCCCTGGAAAAGGCCTTGAATGGG |
| | | TGTCCGCCATCGGCTATGGCGGCGATACCTACTACGCCGACTCTGTG |
| | | AAGGGCAGATTCACCATCAGCCGGGACAACAGCAAGAACACCCTGT |
| | | ACCTGCAGATGAACAGCCTGAGAGCCGAGGACACCGCCGTGTACTAT |
| | | TGCGCCAGAAGGGACGACTACACCAGCAGGGACGCCTTCGATTATTG |
| | | GGGCCAGGGCACACTGGTCACCGTTTCTTCAGCCAGCACCAAGGGCC |
| | | CCAGCGTGTTCCCTCTGGCCCCTTGTAGCAGAAGCACCAGCGAGTCT |
| | | ACAGCCGCCCTGGGCTGCCTCGTGAAGGACTACTTTCCCGAGCCCGT |
| | | GACCGTGTCCTGGAACTCTGGCGCTCTGACAAGCGGCGTGCACACCT |
| | | TTCCAGCCGTGCTGCAGAGCAGCGGCCTGTACTCTCTGAGCAGCGTC |
| | | GTGACAGTGCCCAGCAGCAGCCTGGGCACCAAGACCTACACCTGTAA |
| | | CGTGGACCACAAGCCCAGCAACACCAAGGTGGACAAGCGGGTGGAA |
| | | TCTAAGTACGGCCCTCCCTGCCCTCCTTGCCCAGCCCCTGAATTTCTG |
| | | GGCGGACCCTCCGTGTTCCTGTTCCCCCCAAAGCCCAAGGACACCCT |
| | | GATGATCAGCCGGACCCCCGAAGTGACCTGCGTGGTGGTGGATGTGT |
| | | CCCAGGAAGATCCCGAGGTGCAGTTCAATTGGTACGTGGACGGCGTG |
| | | GAAGTGCACAACGCCAAGACCAAGCCCAGAGAGGAACAGTTCAACA |
| | | GCACCTACCGGGTGGTGTCCGTGCTGACAGTGCTGCACCAGGACTGG |
| | | CTGAACGGCAAAGAGTACAAGTGCAAGGTGTCCAACAAGGGCCTGC |
| | | CCAGCTCCATCGAGAAAACCATCAGCAAGGCCAAGGGCCAGCCCCG |
| | | CGAACCCCAGGTGTACACACTGCCTCCAAGCCAGGAAGAGATGACC |
| | | AAGAACCAGGTGTCCCTGACCTGTCTCGTGAAAGGCTTCTACCCCTC |
| | | CGATATCGCCGTGGAATGGGAGAGCAACGGCCAGCCCGAGAACAAC |
| | | TACAAGACCACCCCCCCTGTGCTGGACAGCGACGGCTCATTCTTCCT |
| | | GTACAGCAGACTGACCGTGGACAAGAGCCGGTGGCAGGAAGGCAAC |
| | | GTGTTCAGCTGCAGCGTGATGCACGAGGCCCTGCACAACCACTACAC |
| | | CCAGAAGTCCCTGTCTCTGAGCCTGGGCAAG |
| 340 | DNA | CAGTCTGTTCTGACACAGCCTCCTAGCGCCTCTGGCACACCTGGACA |
| | | GAGAGTGACCATCAGCTGTAGCGGCAGCAGCTCCAACATCGGCAGC |
| | | AACACCGTGAACTGGTATCAGCAGCTGCCTGGCACAGCCCCTAAACT |
| | | GCTGATCTACTACGACGACCTGCGGCCTAGCGGCGTGCCAGATAGAT |
| | | TTTCTGGCAGCAAGAGCGGCACCTCTGCCAGCCTGGCTATTTCTGGA |
| | | CTGCAGAGCGAGGACGAGGCCGACTACTATTGTCACGCCTGGGACGA |
| | | CAGCCTGAACGTGTACCCTGTTTTTGGCGGAGGCACCAAGCTGACCG |
| | | TTCTAGGCCAGCCTAAAGCCGCCCCTAGCGTGACCCTGTTCCCTCCAA |
| | | GCAGCGAGGAACTGCAGGCCAACAAGGCCACCCTCGTGTGCCTGATC |
| | | AGCGACTTCTATCCTGGCGCCGTGACCGTGGCCTGGAAGGCCGATAG |
| | | CTCTCCTGTGAAGGCCGGCGTGGAAACCACCACCCCTAGCAAGCAGA |
| | | GCAACAACAAATACGCCGCCAGCAGCTACCTGAGCCTGACCCCCGAG |
| | | CAGTGGAAGTCCCACAGATCCTACAGCTGCCAAGTGACCCACGAGGG |
| | | CAGCACCGTGGAAAAGACAGTGGCCCCTACCGAGTGCAGC |
| 341 | PRT | EVQLLESGGGLVQPGGSLRLSCAASGFTFYSYAMSWVRQAPGKGLEWV |
| | | SAIGYGGDTYYADSVKGRFTISRDNSKNTLYLQMNSLRAEDTAVYYCA |
| | | RRDDYTSRDAFDYWGQGTLVTVSS |
| 342 | PRT | SYAMS |
| 343 | PRT | AIGYGGDTYYADSVKG |
| 344 | PRT | RDDYTSRDAFDY |
| 345 | PRT | QSVLTQPPSASGTPGQRVTISCSGSSSNIGSNTVNWYQQLPGTAPKLLIYY |
| | | DDLRPSGVPDRFSGSKSGTSASLAISGLQSEDEADYYCAAWDDSLNDIPV |
| | | FGGGTKLTVL |
| 346 | PRT | SGSSSNIGSNTVN |
| 347 | PRT | YDDLRPS |
| 348 | PRT | AAWDDSLNDIPV |
| 349 | DNA | GAAGTTCAGCTGCTGGAATCTGGCGGCGGACTGGTTCAACCTGGCGG |
| | | ATCTCTGAGACTGAGCTGTGCCGCCAGCGGCTTCACCTTTTACAGCTA |
| | | CGCCATGAGCTGGGTCCGACAGGCCCCTGGAAAAGGCCTTGAATGGG |
| | | TGTCCGCCATCGGCTATGGCGGCGATACCTACTACGCCGACTCTGTG |
| | | AAGGGCAGATTCACCATCAGCCGGGACAACAGCAAGAACACCCTGT |
| | | ACCTGCAGATGAACAGCCTGAGAGCCGAGGACACCGCCGTGTACTAT |
| | | TGCGCCAGAAGGGACGACTACACCAGCAGGGACGCCTTCGATTATTG |
| | | GGGCCAGGGCACACTGGTCACCGTTTCTTCA |
| 350 | DNA | AGCTACGCCATGAGC |
| 351 | DNA | GCCATCGGCTATGGCGGCGATACCTACTACGCCGACTCTGTGAAGGG |
| | | C |
| 352 | DNA | AGGGACGACTACACCAGCAGGGACGCCTTCGATTAT |
| 353 | DNA | CAGTCTGTTCTGACACAGCCTCCTAGCGCCTCTGGCACACCTGGACA |
| | | GAGAGTGACCATCAGCTGTAGCGGCAGCAGCTCCAACATCGGCAGC |
| | | AACACCGTGAACTGGTATCAGCAGCTGCCTGGCACAGCCCCTAAACT |
| | | GCTGATCTACTACGACGACCTGCGGCCTAGCGGCGTGCCAGATAGAT |
| | | TTTCTGGCAGCAAGAGCGGCACCTCTGCCAGCCTGGCTATTTCTGGA |
| | | CTGCAGAGCGAGGACGAGGCCGACTATTATTGTGCCGCCTGGGACGA |
| | | CAGCCTGAACGACATCCCTGTTTTTGGCGGAGGCACCAAGCTGACCG |
| | | TTCTA |
| 354 | DNA | AGCGGCAGCAGCTCCAACATCGGCAGCAACACCGTGAAC |
| 355 | DNA | TACGACGACCTGCGGCCTAGC |
| 356 | DNA | GCCGCCTGGGACGACAGCCTGAACGACATCCCTGTT |
| 357 | PRT | EVQLLESGGGLVQPGGSLRLSCAASGFTFYSYAMSWVRQAPGKGLEWV |
| | | SAIGYGGDTYYADSVKGRFTISRDNSKNTLYLQMNSLRAEDTAVYYCA |
| | | RRDDYTSRDAFDYWGQGTLVTVSSASTKGPSVFPLAPCSRSTSESTAAL |
| | | GCLVKDYFPEPVTVSWNSGALTSGVHTFPAVLQSSGLYSLSSVVTVPSSS |
| | | LGTKTYTCNVDHKPSNTKVDKRVESKYGPPCPPCPAPEFLGGPSVFLFPP |
| | | KPKDTLMISRTPEVTCVVVDVSQEDPEVQFNWYVDGVEVHNAKTKPRE |
| | | EQFNSTYRVVSVLTVLHQDWLNGKEYKCKVSNKGLPSSIEKTISKAKGQ |
| | | PREPQVYTLPPSQEEMTKNQVSLTCLVKGFYPSDIAVEWESNGQPENNY |
| | | KTTPPVLDSDGSFFLYSRLTVDKSRWQEGNVFSCSVMHEALHNHYTQK |
| | | SLSLSLGK |
| 358 | PRT | QSVLTQPPSASGTPGQRVTISCSGSSSNIGSNTVNWYQQLPGTAPKLLIYY |
| | | DDLRPSGVPDRFSGSKSGTSASLAISGLQSEDEADYYCAAWDDSLNDIPV |
| | | FGGGTKLTVLGQPKAAPSVTLFPPSSEELQANKATLVCLISDFYPGAVTV |
| | | AWKADSSPVKAGVETTTPSKQSNNKYAASSYLSLTPEQWKSHRSYSCQ |
| | | VTHEGSTVEKTVAPTECS |
| 359 | DNA | GAAGTTCAGCTGCTGGAATCTGGCGGCGGACTGGTTCAACCTGGCGG |
| | | ATCTCTGAGACTGAGCTGTGCCGCCAGCGGCTTCACCTTTTACAGCTA |
| | | CGCCATGAGCTGGGTCCGACAGGCCCCTGGAAAAGGCCTTGAATGGG |
| | | TGTCCGCCATCGGCTATGGCGGCGATACCTACTACGCCGACTCTGTG |
| | | AAGGGCAGATTCACCATCAGCCGGGACAACAGCAAGAACACCCTGT |
| | | ACCTGCAGATGAACAGCCTGAGAGCCGAGGACACCGCCGTGTACTAT |
| | | TGCGCCAGAAGGGACGACTACACCAGCAGGGACGCCTTCGATTATTG |
| | | GGGCCAGGGCACACTGGTCACCGTTTCTTCAGCCAGCACCAAGGGCC |
| | | CCAGCGTGTTCCCTCTGGCCCCTTGTAGCAGAAGCACCAGCGAGTCT |
| | | ACAGCCGCCCTGGGCTGCCTCGTGAAGGACTACTTTCCCGAGCCCGT |
| | | GACCGTGTCCTGGAACTCTGGCGCTCTGACAAGCGGCGTGCACACCT |
| | | TTCCAGCCGTGCTGCAGAGCAGCGGCCTGTACTCTCTGAGCAGCGTC |
| | | GTGACAGTGCCCAGCAGCAGCCTGGGCACCAAGACCTACACCTGTAA |
| | | CGTGGACCACAAGCCCAGCAACACCAAGGTGGACAAGCGGGTGGAA |
| | | TCTAAGTACGGCCCTCCCTGCCCTCCTTGCCCAGCCCCTGAATTTCTG |
| | | GGCGGACCCTCCGTGTTCCTGTTCCCCCCAAAGCCCAAGGACACCCT |
| | | GATGATCAGCCGGACCCCCGAAGTGACCTGCGTGGTGGTGGATGTGT |
| | | CCCAGGAAGATCCCGAGGTGCAGTTCAATTGGTACGTGGACGGCGTG |
| | | GAAGTGCACAACGCCAAGACCAAGCCCAGAGAGGAACAGTTCAACA |
| | | GCACCTACCGGGTGGTGTCCGTGCTGACAGTGCTGCACCAGGACTGG |
| | | CTGAACGGCAAAGAGTACAAGTGCAAGGTGTCCAACAAGGGCCTGC |
| | | CCAGCTCCATCGAGAAAACCATCAGCAAGGCCAAGGGCCAGCCCCG |
| | | CGAACCCCAGGTGTACACACTGCCTCCAAGCCAGGAAGAGATGACC |
| | | AAGAACCAGGTGTCCCTGACCTGTCTCGTGAAAGGCTTCTACCCCTC |
| | | CGATATCGCCGTGGAATGGGAGAGCAACGGCCAGCCCGAGAACAAC |
| | | TACAAGACCACCCCCCCTGTGCTGGACAGCGACGGCTCATTCTTCCT |
| | | GTACAGCAGACTGACCGTGGACAAGAGCCGGTGGCAGGAAGGCAAC |
| | | GTGTTCAGCTGCAGCGTGATGCACGAGGCCCTGCACAACCACTACAC |
| | | CCAGAAGTCCCTGTCTCTGAGCCTGGGCAAG |
| 360 | DNA | CAGTCTGTTCTGACACAGCCTCCTAGCGCCTCTGGCACACCTGGACA |
| | | GAGAGTGACCATCAGCTGTAGCGGCAGCAGCTCCAACATCGGCAGC |
| | | AACACCGTGAACTGGTATCAGCAGCTGCCTGGCACAGCCCCTAAACT |
| | | GCTGATCTACTACGACGACCTGCGGCCTAGCGGCGTGCCAGATAGAT |
| | | TTTCTGGCAGCAAGAGCGGCACCTCTGCCAGCCTGGCTATTTCTGGA |
| | | CTGCAGAGCGAGGACGAGGCCGACTATTATTGTGCCGCCTGGGACGA |
| | | CAGCCTGAACGACATCCCTGTTTTTGGCGGAGGCACCAAGCTGACCG |
| | | TTCTAGGCCAGCCTAAAGCCGCCCCTAGCGTGACCCTGTTCCCTCCAA |
| | | GCAGCGAGGAACTGCAGGCCAACAAGGCCACCCTCGTGTGCCTGATC |
| | | AGCGACTTCTATCCTGGCGCCGTGACCGTGGCCTGGAAGGCCGATAG |
| | | CTCTCCTGTGAAGGCCGGCGTGGAAACCACCACCCCTAGCAAGCAGA |
| | | GCAACAACAAATACGCCGCCAGCAGCTACCTGAGCCTGACCCCCGAG |
| | | CAGTGGAAGTCCCACAGATCCTACAGCTGCCAAGTGACCCACGAGGG |
| | | CAGCACCGTGGAAAAGACAGTGGCCCCTACCGAGTGCAGC |
| 361 | PRT | EVQLLESGGGLVQPGGSLRLSCAASGFTFYSYAMSWVRQAPGKGLEWV |
| | | SAIGYGGDTYYADSVKGRFTISRDNSKNTLYLQMNSLRAEDTAVYYCA |
| | | RRDDYTSRDAFDYWGQGTLVTVSS |
| 362 | PRT | SYAMS |
| 363 | PRT | AIGYGGDTYYADSVKG |
| 364 | PRT | RDDYTSRDAFDY |
| 365 | PRT | QSVLTQPPSASGTPGQRVTISCSGSSSNIGSNTVNWYQQLPGTAPKLLIYY |
| | | DDLRPSGVPDRFSGSKSGTSASLAISGLQSEDEADYYCAAWDDSLNVIPV |
| | | FGGGTKLTVL |
| 366 | PRT | SGSSSNIGSNTVN |
| 367 | PRT | YDDLRPS |
| 368 | PRT | AAWDDSLNVIPV |
| 369 | DNA | GAAGTTCAGCTGCTGGAATCTGGCGGCGGACTGGTTCAACCTGGCGG |
| | | ATCTCTGAGACTGAGCTGTGCCGCCAGCGGCTTCACCTTTTACAGCTA |
| | | CGCCATGAGCTGGGTCCGACAGGCCCCTGGAAAAGGCCTTGAATGGG |
| | | TGTCCGCCATCGGCTATGGCGGCGATACCTACTACGCCGACTCTGTG |
| | | AAGGGCAGATTCACCATCAGCCGGGACAACAGCAAGAACACCCTGT |
| | | ACCTGCAGATGAACAGCCTGAGAGCCGAGGACACCGCCGTGTACTAT |
| | | TGCGCCAGAAGGGACGACTACACCAGCAGGGACGCCTTCGATTATTG |
| | | GGGCCAGGGCACACTGGTCACCGTTTCTTCA |
| 370 | DNA | AGCTACGCCATGAGC |
| 371 | DNA | GCCATCGGCTATGGCGGCGATACCTACTACGCCGACTCTGTGAAGGG |
| | | C |
| 372 | DNA | AGGGACGACTACACCAGCAGGGACGCCTTCGATTAT |
| 373 | DNA | CAGTCTGTTCTGACACAGCCTCCTAGCGCCTCTGGCACACCTGGACA |
| | | GAGAGTGACCATCAGCTGTAGCGGCAGCAGCTCCAACATCGGCAGC |
| | | AACACCGTGAACTGGTATCAGCAGCTGCCTGGCACAGCCCCTAAACT |
| | | GCTGATCTACTACGACGACCTGCGGCCTAGCGGCGTGCCAGATAGAT |
| | | TTTCTGGCAGCAAGAGCGGCACCTCTGCCAGCCTGGCTATTTCTGGA |
| | | CTGCAGAGCGAGGACGAGGCCGACTATTATTGTGCCGCCTGGGACGA |
| | | CAGCCTGAACGTGATCCCTGTTTTTGGCGGAGGCACCAAGCTGACCG |
| | | TTCTA |
| 374 | DNA | AGCGGCAGCAGCTCCAACATCGGCAGCAACACCGTGAAC |
| 375 | DNA | TACGACGACCTGCGGCCTAGC |
| 376 | DNA | GCCGCCTGGGACGACAGCCTGAACGTGATCCCTGTT |
| 377 | PRT | EVQLLESGGGLVQPGGSLRLSCAASGFTFYSYAMSWVRQAPGKGLEWV |
| | | SAIGYGGDTYYADSVKGRFTISRDNSKNTLYLQMNSLRAEDTAVYYCA |
| | | RRDDYTSRDAFDYWGQGTLVTVSSASTKGPSVFPLAPCSRSTSESTAAL |
| | | GCLVKDYFPEPVTVSWNSGALTSGVHTFPAVLQSSGLYSLSSVVTVPSSS |
| | | LGTKTYTCNVDHKPSNTKVDKRVESKYGPPCPPCPAPEFLGGPSVFLFPP |
| | | KPKDTLMISRTPEVTCVVVDVSQEDPEVQFNWYVDGVEVHNAKTKPRE |
| | | EQFNSTYRVVSVLTVLHQDWLNGKEYKCKVSNKGLPSSIEKTISKAKGQ |
| | | PREPQVYTLPPSQEEMTKNQVSLTCLVKGFYPSDIAVEWESNGQPENNY |
| | | KTTPPVLDSDGSFFLYSRLTVDKSRWQEGNVFSCSVMHEALHNHYTQK |
| | | SLSLSLGK |
| 378 | PRT | QSVLTQPPSASGTPGQRVTISCSGSSSNIGSNTVNWYQQLPGTAPKLLIYY |
| | | DDLRPSGVPDRFSGSKSGTSASLAISGLQSEDEADYYCAAWDDSLNVIPV |
| | | FGGGTKLTVLGQPKAAPSVTLFPPSSEELQANKATLVCLISDFYPGAVTV |
| | | AWKADSSPVKAGVETTTPSKQSNNKYAASSYLSLTPEQWKSHRSYSCQ |
| | | VTHEGSTVEKTVAPTECS |
| 379 | DNA | GAAGTTCAGCTGCTGGAATCTGGCGGCGGACTGGTTCAACCTGGCGG |
| | | ATCTCTGAGACTGAGCTGTGCCGCCAGCGGCTTCACCTTTTACAGCTA |
| | | CGCCATGAGCTGGGTCCGACAGGCCCCTGGAAAAGGCCTTGAATGGG |
| | | TGTCCGCCATCGGCTATGGCGGCGATACCTACTACGCCGACTCTGTG |
| | | AAGGGCAGATTCACCATCAGCCGGGACAACAGCAAGAACACCCTGT |
| | | ACCTGCAGATGAACAGCCTGAGAGCCGAGGACACCGCCGTGTACTAT |
| | | TGCGCCAGAAGGGACGACTACACCAGCAGGGACGCCTTCGATTATTG |
| | | GGGCCAGGGCACACTGGTCACCGTTTCTTCAGCCAGCACCAAGGGCC |
| | | CCAGCGTGTTCCCTCTGGCCCCTTGTAGCAGAAGCACCAGCGAGTCT |
| | | ACAGCCGCCCTGGGCTGCCTCGTGAAGGACTACTTTCCCGAGCCCGT |
| | | GACCGTGTCCTGGAACTCTGGCGCTCTGACAAGCGGCGTGCACACCT |
| | | TTCCAGCCGTGCTGCAGAGCAGCGGCCTGTACTCTCTGAGCAGCGTC |
| | | GTGACAGTGCCCAGCAGCAGCCTGGGCACCAAGACCTACACCTGTAA |
| | | CGTGGACCACAAGCCCAGCAACACCAAGGTGGACAAGCGGGTGGAA |
| | | TCTAAGTACGGCCCTCCCTGCCCTCCTTGCCCAGCCCCTGAATTTCTG |
| | | GGCGGACCCTCCGTGTTCCTGTTCCCCCCAAAGCCCAAGGACACCCT |
| | | GATGATCAGCCGGACCCCCGAAGTGACCTGCGTGGTGGTGGATGTGT |
| | | CCCAGGAAGATCCCGAGGTGCAGTTCAATTGGTACGTGGACGGCGTG |
| | | GAAGTGCACAACGCCAAGACCAAGCCCAGAGAGGAACAGTTCAACA |
| | | GCACCTACCGGGTGGTGTCCGTGCTGACAGTGCTGCACCAGGACTGG |
| | | CTGAACGGCAAAGAGTACAAGTGCAAGGTGTCCAACAAGGGCCTGC |
| | | CCAGCTCCATCGAGAAAACCATCAGCAAGGCCAAGGGCCAGCCCCG |
| | | CGAACCCCAGGTGTACACACTGCCTCCAAGCCAGGAAGAGATGACC |
| | | AAGAACCAGGTGTCCCTGACCTGTCTCGTGAAAGGCTTCTACCCCTC |
| | | CGATATCGCCGTGGAATGGGAGAGCAACGGCCAGCCCGAGAACAAC |
| | | TACAAGACCACCCCCCCTGTGCTGGACAGCGACGGCTCATTCTTCCT |
| | | GTACAGCAGACTGACCGTGGACAAGAGCCGGTGGCAGGAAGGCAAC |
| | | GTGTTCAGCTGCAGCGTGATGCACGAGGCCCTGCACAACCACTACAC |
| | | CCAGAAGTCCCTGTCTCTGAGCCTGGGCAAG |
| 380 | DNA | CAGTCTGTTCTGACACAGCCTCCTAGCGCCTCTGGCACACCTGGACA |
| | | GAGAGTGACCATCAGCTGTAGCGGCAGCAGCTCCAACATCGGCAGC |
| | | AACACCGTGAACTGGTATCAGCAGCTGCCTGGCACAGCCCCTAAACT |
| | | GCTGATCTACTACGACGACCTGCGGCCTAGCGGCGTGCCAGATAGAT |
| | | TTTCTGGCAGCAAGAGCGGCACCTCTGCCAGCCTGGCTATTTCTGGA |
| | | CTGCAGAGCGAGGACGAGGCCGACTATTATTGTGCCGCCTGGGACGA |
| | | CAGCCTGAACGTGATCCCTGTTTTTGGCGGAGGCACCAAGCTGACCG |
| | | TTCTAGGCCAGCCTAAAGCCGCCCCTAGCGTGACCCTGTTCCCTCCAA |
| | | GCAGCGAGGAACTGCAGGCCAACAAGGCCACCCTCGTGTGCCTGATC |
| | | AGCGACTTCTATCCTGGCGCCGTGACCGTGGCCTGGAAGGCCGATAG |
| | | CTCTCCTGTGAAGGCCGGCGTGGAAACCACCACCCCTAGCAAGCAGA |
| | | GCAACAACAAATACGCCGCCAGCAGCTACCTGAGCCTGACCCCCGAG |
| | | CAGTGGAAGTCCCACAGATCCTACAGCTGCCAAGTGACCCACGAGGG |
| | | CAGCACCGTGGAAAAGACAGTGGCCCCTACCGAGTGCAGC |
| 381 | PRT | EVQLLESGGGLVQPGGSLRLSCAASGFTFDSYEMNWVRQAPGKGLEWV |
| | | SGISWNSGWIDYADSVKGRFTISRDNSKNTLYLQMNSLRAEDTAVYYC |
| | | ARSGYSSSWFDPDFDYWGQGTLVTVSS |
| 382 | PRT | SYEMN |
| 383 | PRT | GISWNSGWIDYADSVKG |
| 384 | PRT | SGYSSSWFDPDFDY |
| 385 | PRT | QSVLTQPPSVSGAPGQRVTISCTGSSSDIGAGYDVHWYQQLPGTAPKLLI |
| | | YGNSNRPSGVPDRFSGSKSGTSASLAITGLQAEDEADYYCSSYAGPNPY |
| | | VVFGGGTKLTVL |
| 386 | PRT | TGSSSDIGAGYDVH |
| 387 | PRT | GNSNRPS |
| 388 | PRT | SSYAGPNPYVV |
| 389 | DNA | GAAGTTCAGCTGCTGGAATCTGGCGGCGGACTGGTTCAACCTGGCGG |
| | | ATCTCTGAGACTGAGCTGTGCCGCCAGCGGCTTCACCTTCGATAGCT |
| | | ACGAGATGAACTGGGTCCGACAGGCCCCTGGCAAAGGCCTTGAATG |
| | | GGTGTCCGGCATCAGCTGGAATAGCGGCTGGATCGACTACGCCGACA |
| | | GCGTGAAGGGCAGATTCACCATCAGCCGGGACAACAGCAAGAACAC |
| | | CCTGTACCTGCAGATGAACAGCCTGAGAGCCGAGGACACCGCCGTGT |
| | | ACTACTGTGCCAGAAGCGGCTACAGCAGCTCTTGGTTTGACCCCGAC |
| | | TTCGACTATTGGGGCCAGGGCACACTGGTCACAGTCTCTTCA |
| 390 | DNA | AGCTACGAGATGAAC |
| 391 | DNA | GGCATCAGCTGGAATAGCGGCTGGATCGACTACGCCGACAGCGTGA |
| | | AGGGC |
| 392 | DNA | AGCGGCTACAGCAGCTCTTGGTTTGACCCCGACTTCGACTAT |
| 393 | DNA | CAGTCTGTTCTGACACAGCCTCCATCTGTGTCTGGCGCCCCTGGACAG |
| | | AGAGTGACCATCAGCTGTACAGGCAGCAGCTCCGATATTGGCGCCGG |
| | | ATACGACGTGCACTGGTATCAGCAACTGCCTGGCACAGCCCCTAAGC |
| | | TGCTGATCTACGGCAACAGCAACAGACCTAGCGGCGTGCCCGATAGA |
| | | TTCAGCGGCTCTAAGTCTGGCACAAGCGCCAGCCTGGCCATTACTGG |
| | | ACTGCAGGCCGAAGATGAGGCCGACTACTACTGCAGCAGCTACGCTG |
| | | GCCCCAATCCTTACGTGGTGTTTGGCGGCGGAACAAAGCTGACCGTT |
| | | CTA |
| 394 | DNA | ACAGGCAGCAGCTCCGATATTGGCGCCGGATACGACGTGCAC |
| 395 | DNA | GGCAACAGCAACAGACCTAGC |
| 396 | DNA | AGCAGCTACGCTGGCCCCAATCCTTACGTGGTG |
| 397 | PRT | EVQLLESGGGLVQPGGSLRLSCAASGFTFDSYEMNWVRQAPGKGLEWV |
| | | SGISWNSGWIDYADSVKGRFTISRDNSKNTLYLQMNSLRAEDTAVYYC |
| | | ARSGYSSSWFDPDFDYWGQGTLVTVSSASTKGPSVFPLAPSSKSTSGGT |
| | | AALGCLVKDYFPEPVTVSWNSGALTSGVHTFPAVLQSSGLYSLSSVVTV |
| | | PSSSLGTQTYICNVNHKPSNTKVDKKVEPKSCDKTHTCPPCPAPELLGGP |
| | | SVFLFPPKPKDTLMISRTPEVTCVVVDVSHEDPEVKFNWYVDGVEVHNA |
| | | KTKPREEQYNSTYRVVSVLTVLHQDWLNGKEYKCKVSNKALPAPIEKTI |
| | | SKAKGQPREPQVYTLPPSRDELTKNQVSLTCLVKGFYPSDIAVEWESNG |
| | | QPENNYKTTPPVLDSDGSFFLYSKLTVDKSRWQQGNVFSCSVMHEALH |
| | | NHYTQKSLSLSPGK |
| 398 | PRT | QSVLTQPPSVSGAPGQRVTISCTGSSSDIGAGYDVHWYQQLPGTAPKLLI |
| | | YGNSNRPSGVPDRFSGSKSGTSASLAITGLQAEDEADYYCSSYAGPNPY |
| | | VVFGGGTKLTVLGQPKAAPSVTLFPPSSEELQANKATLVCLISDFYPGAV |
| | | TVAWKADSSPVKAGVETTTPSKQSNNKYAASSYLSLTPEQWKSHRSYS |
| | | CQVTHEGSTVEKTVAPTECS |
| 399 | DNA | GAAGTTCAGCTGCTGGAATCTGGCGGCGGACTGGTTCAACCTGGCGG |
| | | ATCTCTGAGACTGAGCTGTGCCGCCAGCGGCTTCACCTTCGATAGCT |
| | | ACGAGATGAACTGGGTCCGACAGGCCCCTGGCAAAGGCCTTGAATG |
| | | GGTGTCCGGCATCAGCTGGAATAGCGGCTGGATCGACTACGCCGACA |
| | | GCGTGAAGGGCAGATTCACCATCAGCCGGGACAACAGCAAGAACAC |
| | | CCTGTACCTGCAGATGAACAGCCTGAGAGCCGAGGACACCGCCGTGT |
| | | ACTACTGTGCCAGAAGCGGCTACAGCAGCTCTTGGTTTGACCCCGAC |
| | | TTCGACTATTGGGGCCAGGGCACACTGGTCACAGTCTCTTCAGCCAG |
| | | CACCAAGGGCCCCAGCGTGTTCCCTCTGGCCCCTAGCAGCAAGAGCA |
| | | CATCTGGCGGAACAGCCGCCCTGGGCTGCCTCGTGAAGGACTACTTT |
| | | CCCGAGCCCGTGACCGTGTCCTGGAACTCTGGCGCTCTGACAAGCGG |
| | | CGTGCACACCTTTCCAGCCGTGCTGCAGAGCAGCGGCCTGTACTCTCT |
| | | GAGCAGCGTCGTGACAGTGCCCAGCAGCTCTCTGGGCACCCAGACCT |
| | | ACATCTGCAACGTGAACCACAAGCCCAGCAACACCAAGGTGGACAA |
| | | GAAGGTGGAACCCAAGAGCTGCGACAAGACCCACACCTGTCCCCCTT |
| | | GTCCTGCCCCCGAACTGCTGGGAGGCCCTTCCGTGTTCCTGTTCCCCC |
| | | CAAAGCCCAAGGACACCCTGATGATCAGCCGGACCCCCGAAGTGAC |
| | | CTGCGTGGTGGTGGATGTGTCCCACGAGGACCCTGAAGTGAAGTTCA |
| | | ATTGGTACGTGGACGGCGTGGAAGTGCACAACGCCAAGACCAAGCC |
| | | TAGAGAGGAACAGTACAACAGCACCTACCGGGTGGTGTCCGTGCTGA |
| | | CAGTGCTGCACCAGGACTGGCTGAACGGCAAAGAGTACAAGTGCAA |
| | | GGTGTCCAACAAGGCCCTGCCTGCCCCCATCGAGAAAACCATCAGCA |
| | | AGGCCAAGGGCCAGCCCCGCGAACCCCAGGTGTACACACTGCCCCCA |
| | | AGCAGGGACGAGCTGACCAAGAACCAGGTGTCCCTGACCTGTCTCGT |
| | | GAAAGGCTTCTACCCCTCCGATATCGCCGTGGAATGGGAGAGCAACG |
| | | GCCAGCCCGAGAACAACTACAAGACCACCCCCCCTGTGCTGGACAGC |
| | | GACGGCTCATTCTTCCTGTACAGCAAGCTGACCGTGGACAAGTCCCG |
| | | GTGGCAGCAGGGCAACGTGTTCAGCTGCAGCGTGATGCACGAGGCCC |
| | | TGCACAACCACTACACCCAGAAGTCCCTGAGCCTGAGCCCTGGCAAG |
| 400 | DNA | CAGTCTGTTCTGACACAGCCTCCATCTGTGTCTGGCGCCCCTGGACAG |
| | | AGAGTGACCATCAGCTGTACAGGCAGCAGCTCCGATATTGGCGCCGG |
| | | ATACGACGTGCACTGGTATCAGCAACTGCCTGGCACAGCCCCTAAGC |
| | | TGCTGATCTACGGCAACAGCAACAGACCTAGCGGCGTGCCCGATAGA |
| | | TTCAGCGGCTCTAAGTCTGGCACAAGCGCCAGCCTGGCCATTACTGG |
| | | ACTGCAGGCCGAAGATGAGGCCGACTACTACTGCAGCAGCTACGCTG |
| | | GCCCCAATCCTTACGTGGTGTTTGGCGGCGGAACAAAGCTGACCGTT |
| | | CTAGGCCAGCCTAAAGCCGCCCCTAGCGTGACCCTGTTCCCTCCAAG |
| | | CAGCGAGGAACTGCAGGCCAACAAGGCCACCCTCGTGTGCCTGATCA |
| | | GCGACTTCTATCCTGGCGCCGTGACCGTGGCCTGGAAGGCCGATAGC |
| | | TCTCCTGTGAAGGCCGGCGTGGAAACCACCACCCCTAGCAAGCAGAG |
| | | CAACAACAAATACGCCGCCAGCAGCTACCTGAGCCTGACCCCCGAGC |
| | | AGTGGAAGTCCCACAGATCCTACAGCTGCCAAGTGACCCACGAGGGC |
| | | AGCACCGTGGAAAAGACAGTGGCCCCTACCGAGTGCAGC |
| 401 | PRT | EVQLLESGGGLVQPGGSLRLSCAASGFTFDSYEMNWVRQAPGKGLEWV |
| | | SGISWNSGWIDYADSVKGRFTISRDNSKNTLYLQMNSLRAEDTAVYYC |
| | | ARSGYSSSWFDPDFDYWGQGTLVTVSS |
| 402 | PRT | SYEMN |
| 403 | PRT | GISWNSGWIDYADSVKG |
| 404 | PRT | SGYSSSWFDPDFDY |
| 405 | PRT | QSVLTQPPSVSGAPGQRVTISCTGSSSNIGAGYDVHWYQQLPGTAPKLLI |
| | | YGNSNRPSGVPDRFSGSKSGTSASLAITGLQAEDEADYYCQSYAGINPY |
| | | VVFGGGTKLTVL |
| 406 | PRT | TGSSSNIGAGYDVH |
| 407 | PRT | GNSNRPS |
| 408 | PRT | QSYAGINPYVV |
| 409 | DNA | GAAGTTCAGCTGCTGGAATCTGGCGGCGGACTGGTTCAACCTGGCGG |
| | | ATCTCTGAGACTGAGCTGTGCCGCCAGCGGCTTCACCTTCGATAGCT |
| | | ACGAGATGAACTGGGTCCGACAGGCCCCTGGCAAAGGCCTTGAATG |
| | | GGTGTCCGGCATCAGCTGGAATAGCGGCTGGATCGACTACGCCGACA |
| | | GCGTGAAGGGCAGATTCACCATCAGCCGGGACAACAGCAAGAACAC |
| | | CCTGTACCTGCAGATGAACAGCCTGAGAGCCGAGGACACCGCCGTGT |
| | | ACTACTGTGCCAGAAGCGGCTACAGCAGCTCTTGGTTTGACCCCGAC |
| | | TTCGACTATTGGGGCCAGGGCACACTGGTCACAGTCTCTTCA |
| 410 | DNA | AGCTACGAGATGAAC |
| 411 | DNA | GGCATCAGCTGGAATAGCGGCTGGATCGACTACGCCGACAGCGTGA |
| | | AGGGC |
| 412 | DNA | AGCGGCTACAGCAGCTCTTGGTTTGACCCCGACTTCGACTAT |
| 413 | DNA | CAGTCTGTTCTGACACAGCCTCCATCTGTGTCTGGCGCCCCTGGACAG |
| | | AGAGTGACCATCAGCTGTACAGGCAGCAGCTCCAATATCGGAGCCGG |
| | | CTATGACGTGCACTGGTATCAGCAGCTGCCTGGCACAGCCCCTAAAC |
| | | TGCTGATCTACGGCAACAGCAACAGACCCAGCGGCGTGCCCGATAGA |
| | | TTTTCCGGCTCTAAGAGCGGCACAAGCGCCAGCCTGGCTATTACTGG |
| | | ACTGCAGGCCGAGGACGAGGCCGACTACTACTGTCAGAGCTACGCCG |
| | | GCATCAACCCCTACGTGGTGTTTGGCGGAGGCACCAAGCTGACAGTT |
| | | CTA |
| 414 | DNA | ACAGGCAGCAGCTCCAATATCGGAGCCGGCTATGACGTGCAC |
| 415 | DNA | GGCAACAGCAACAGACCCAGC |
| 416 | DNA | CAGAGCTACGCCGGCATCAACCCCTACGTGGTG |
| 417 | PRT | EVQLLESGGGLVQPGGSLRLSCAASGFTFDSYEMNWVRQAPGKGLEWV |
| | | SGISWNSGWIDYADSVKGRFTISRDNSKNTLYLQMNSLRAEDTAVYYC |
| | | ARSGYSSSWFDPDFDYWGQGTLVTVSSASTKGPSVFPLAPSSKSTSGGT |
| | | AALGCLVKDYFPEPVTVSWNSGALTSGVHTFPAVLQSSGLYSLSSVVTV |
| | | PSSSLGTQTYICNVNHKPSNTKVDKKVEPKSCDKTHTCPPCPAPELLGGP |
| | | SVFLFPPKPKDTLMISRTPEVTCVVVDVSHEDPEVKFNWYVDGVEVHNA |
| | | KTKPREEQYNSTYRVVSVLTVLHQDWLNGKEYKCKVSNKALPAPIEKTI |
| | | SKAKGQPREPQVYTLPPSRDELTKNQVSLTCLVKGFYPSDIAVEWESNG |
| | | QPENNYKTTPPVLDSDGSFFLYSKLTVDKSRWQQGNVFSCSVMHEALH |
| | | NHYTQKSLSLSPGK |
| 418 | PRT | QSVLTQPPSVSGAPGQRVTISCTGSSSNIGAGYDVHWYQQLPGTAPKLLI |
| | | YGNSNRPSGVPDRFSGSKSGTSASLAITGLQAEDEADYYCQSYAGINPY |
| | | VVFGGGTKLTVLGQPKAAPSVTLFPPSSEELQANKATLVCLISDFYPGAV |
| | | TVAWKADSSPVKAGVETTTPSKQSNNKYAASSYLSLTPEQWKSHRSYS |
| | | CQVTHEGSTVEKTVAPTECS |
| 419 | DNA | GAAGTTCAGCTGCTGGAATCTGGCGGCGGACTGGTTCAACCTGGCGG |
| | | ATCTCTGAGACTGAGCTGTGCCGCCAGCGGCTTCACCTTCGATAGCT |
| | | ACGAGATGAACTGGGTCCGACAGGCCCCTGGCAAAGGCCTTGAATG |
| | | GGTGTCCGGCATCAGCTGGAATAGCGGCTGGATCGACTACGCCGACA |
| | | GCGTGAAGGGCAGATTCACCATCAGCCGGGACAACAGCAAGAACAC |
| | | CCTGTACCTGCAGATGAACAGCCTGAGAGCCGAGGACACCGCCGTGT |
| | | ACTACTGTGCCAGAAGCGGCTACAGCAGCTCTTGGTTTGACCCCGAC |
| | | TTCGACTATTGGGGCCAGGGCACACTGGTCACAGTCTCTTCAGCCAG |
| | | CACCAAGGGCCCCAGCGTGTTCCCTCTGGCCCCTAGCAGCAAGAGCA |
| | | CATCTGGCGGAACAGCCGCCCTGGGCTGCCTCGTGAAGGACTACTTT |
| | | CCCGAGCCCGTGACCGTGTCCTGGAACTCTGGCGCTCTGACAAGCGG |
| | | CGTGCACACCTTTCCAGCCGTGCTGCAGAGCAGCGGCCTGTACTCTCT |
| | | GAGCAGCGTCGTGACAGTGCCCAGCAGCTCTCTGGGCACCCAGACCT |
| | | ACATCTGCAACGTGAACCACAAGCCCAGCAACACCAAGGTGGACAA |
| | | GAAGGTGGAACCCAAGAGCTGCGACAAGACCCACACCTGTCCCCCTT |
| | | GTCCTGCCCCCGAACTGCTGGGAGGCCCTTCCGTGTTCCTGTTCCCCC |
| | | CAAAGCCCAAGGACACCCTGATGATCAGCCGGACCCCCGAAGTGAC |
| | | CTGCGTGGTGGTGGATGTGTCCCACGAGGACCCTGAAGTGAAGTTCA |
| | | ATTGGTACGTGGACGGCGTGGAAGTGCACAACGCCAAGACCAAGCC |
| | | TAGAGAGGAACAGTACAACAGCACCTACCGGGTGGTGTCCGTGCTGA |
| | | CAGTGCTGCACCAGGACTGGCTGAACGGCAAAGAGTACAAGTGCAA |
| | | GGTGTCCAACAAGGCCCTGCCTGCCCCCATCGAGAAAACCATCAGCA |
| | | AGGCCAAGGGCCAGCCCCGCGAACCCCAGGTGTACACACTGCCCCCA |
| | | AGCAGGGACGAGCTGACCAAGAACCAGGTGTCCCTGACCTGTCTCGT |
| | | GAAAGGCTTCTACCCCTCCGATATCGCCGTGGAATGGGAGAGCAACG |
| | | GCCAGCCCGAGAACAACTACAAGACCACCCCCCCTGTGCTGGACAGC |
| | | GACGGCTCATTCTTCCTGTACAGCAAGCTGACCGTGGACAAGTCCCG |
| | | GTGGCAGCAGGGCAACGTGTTCAGCTGCAGCGTGATGCACGAGGCCC |
| | | TGCACAACCACTACACCCAGAAGTCCCTGAGCCTGAGCCCTGGCAAG |
| 420 | DNA | CAGTCTGTTCTGACACAGCCTCCATCTGTGTCTGGCGCCCCTGGACAG |
| | | AGAGTGACCATCAGCTGTACAGGCAGCAGCTCCAATATCGGAGCCGG |
| | | CTATGACGTGCACTGGTATCAGCAGCTGCCTGGCACAGCCCCTAAAC |
| | | TGCTGATCTACGGCAACAGCAACAGACCCAGCGGCGTGCCCGATAGA |
| | | TTTTCCGGCTCTAAGAGCGGCACAAGCGCCAGCCTGGCTATTACTGG |
| | | ACTGCAGGCCGAGGACGAGGCCGACTACTACTGTCAGAGCTACGCCG |
| | | GCATCAACCCCTACGTGGTGTTTGGCGGAGGCACCAAGCTGACAGTT |
| | | CTAGGCCAGCCTAAAGCCGCCCCTAGCGTGACCCTGTTCCCTCCAAG |
| | | CAGCGAGGAACTGCAGGCCAACAAGGCCACCCTCGTGTGCCTGATCA |
| | | GCGACTTCTATCCTGGCGCCGTGACCGTGGCCTGGAAGGCCGATAGC |
| | | TCTCCTGTGAAGGCCGGCGTGGAAACCACCACCCCTAGCAAGCAGAG |
| | | CAACAACAAATACGCCGCCAGCAGCTACCTGAGCCTGACCCCCGAGC |
| | | AGTGGAAGTCCCACAGATCCTACAGCTGCCAAGTGACCCACGAGGGC |
| | | AGCACCGTGGAAAAGACAGTGGCCCCTACCGAGTGCAGC |
| 421 | PRT | EVQLLESGGGLVQPGGSLRLSCAASGFDFSSYEMNWVRQAPGKGLEWV |
| | | SGISWNSGWIGYADSVKGRFTISRDNSKNTLYLQMNSLRAEDTAVYYC |
| | | ARSGYSSSWFDPDFDYWGQGTLVTVSS |
| 422 | PRT | SYEMN |
| 423 | PRT | GISWNSGWIGYADSVKG |
| 424 | PRT | SGYSSSWFDPDFDY |
| 425 | PRT | QSVLTQPPSVSGAPGQRVTISCTGSSSNIGAGYDVHWYQQLPGTAPKLLI |
| | | YGNSNRPSGVPDRFSGSKSGTSASLAITGLQAEDEADYYCQSYAGPNPY |
| | | VVFGGGTKLTVL |
| 426 | PRT | TGSSSNIGAGYDVH |
| 427 | PRT | GNSNRPS |
| 428 | PRT | QSYAGPNPYVV |
| 429 | DNA | GAAGTTCAGCTGCTGGAATCTGGCGGCGGACTGGTTCAACCTGGCGG |
| | | ATCTCTGAGACTGAGCTGTGCCGCCAGCGGCTTCGATTTCAGCAGCT |
| | | ACGAGATGAACTGGGTCCGACAGGCCCCTGGCAAAGGCCTTGAATG |
| | | GGTGTCCGGCATCAGCTGGAATAGCGGCTGGATCGGCTACGCCGATA |
| | | GCGTGAAGGGCAGATTCACCATCAGCCGGGACAACAGCAAGAACAC |
| | | CCTGTACCTGCAGATGAACAGCCTGAGAGCCGAGGACACCGCCGTGT |
| | | ACTACTGTGCCAGAAGCGGCTACAGCAGCTCTTGGTTTGACCCCGAC |
| | | TTCGACTATTGGGGCCAGGGCACACTGGTCACAGTCTCTTCA |
| 430 | DNA | AGCTACGAGATGAAC |
| 431 | DNA | GGCATCAGCTGGAATAGCGGCTGGATCGGCTACGCCGATAGCGTGAA |
| | | GGGC |
| 432 | DNA | AGCGGCTACAGCAGCTCTTGGTTTGACCCCGACTTCGACTAT |
| 433 | DNA | CAGTCTGTTCTGACACAGCCTCCATCTGTGTCTGGCGCCCCTGGACAG |
| | | AGAGTGACCATCAGCTGTACAGGCAGCAGCTCCAATATCGGAGCCGG |
| | | CTATGACGTGCACTGGTATCAGCAGCTGCCTGGCACAGCCCCTAAAC |
| | | TGCTGATCTACGGCAACAGCAACAGACCCAGCGGCGTGCCCGATAGA |
| | | TTTTCCGGCTCTAAGAGCGGCACAAGCGCCAGCCTGGCTATTACTGG |
| | | ACTGCAGGCCGAGGACGAGGCCGACTACTACTGTCAGTCTTACGCTG |
| | | GCCCCAATCCTTACGTGGTGTTTGGCGGCGGAACAAAGCTGACCGTT |
| | | CTA |
| 434 | DNA | ACAGGCAGCAGCTCCAATATCGGAGCCGGCTATGACGTGCAC |
| 435 | DNA | GGCAACAGCAACAGACCCAGC |
| 436 | DNA | CAGTCTTACGCTGGCCCCAATCCTTACGTGGTG |
| 437 | PRT | EVQLLESGGGLVQPGGSLRLSCAASGFDFSSYEMNWVRQAPGKGLEWV |
| | | SGISWNSGWIGYADSVKGRFTISRDNSKNTLYLQMNSLRAEDTAVYYC |
| | | ARSGYSSSWFDPDFDYWGQGTLVTVSSASTKGPSVFPLAPSSKSTSGGT |
| | | AALGCLVKDYFPEPVTVSWNSGALTSGVHTFPAVLQSSGLYSLSSVVTV |
| | | PSSSLGTQTYICNVNHKPSNTKVDKKVEPKSCDKTHTCPPCPAPELLGGP |
| | | SVFLFPPKPKDTLMISRTPEVTCVVVDVSHEDPEVKFNWYVDGVEVHNA |
| | | KTKPREEQYNSTYRVVSVLTVLHQDWLNGKEYKCKVSNKALPAPIEKTI |
| | | SKAKGQPREPQVYTLPPSRDELTKNQVSLTCLVKGFYPSDIAVEWESNG |
| | | QPENNYKTTPPVLDSDGSFFLYSKLTVDKSRWQQGNVFSCSVMHEALH |
| | | NHYTQKSLSLSPGK |
| 438 | PRT | QSVLTQPPSVSGAPGQRVTISCTGSSSNIGAGYDVHWYQQLPGTAPKLLI |
| | | YGNSNRPSGVPDRFSGSKSGTSASLAITGLQAEDEADYYCQSYAGPNPY |
| | | VVFGGGTKLTVLGQPKAAPSVTLFPPSSEELQANKATLVCLISDFYPGAV |
| | | TVAWKADSSPVKAGVETTTPSKQSNNKYAASSYLSLTPEQWKSHRSYS |
| | | CQVTHEGSTVEKTVAPTECS |
| 439 | DNA | GAAGTTCAGCTGCTGGAATCTGGCGGCGGACTGGTTCAACCTGGCGG |
| | | ATCTCTGAGACTGAGCTGTGCCGCCAGCGGCTTCGATTTCAGCAGCT |
| | | ACGAGATGAACTGGGTCCGACAGGCCCCTGGCAAAGGCCTTGAATG |
| | | GGTGTCCGGCATCAGCTGGAATAGCGGCTGGATCGGCTACGCCGATA |
| | | GCGTGAAGGGCAGATTCACCATCAGCCGGGACAACAGCAAGAACAC |
| | | CCTGTACCTGCAGATGAACAGCCTGAGAGCCGAGGACACCGCCGTGT |
| | | ACTACTGTGCCAGAAGCGGCTACAGCAGCTCTTGGTTTGACCCCGAC |
| | | TTCGACTATTGGGGCCAGGGCACACTGGTCACAGTCTCTTCAGCCAG |
| | | CACCAAGGGCCCCAGCGTGTTCCCTCTGGCCCCTAGCAGCAAGAGCA |
| | | CATCTGGCGGAACAGCCGCCCTGGGCTGCCTCGTGAAGGACTACTTT |
| | | CCCGAGCCCGTGACCGTGTCCTGGAACTCTGGCGCTCTGACAAGCGG |
| | | CGTGCACACCTTTCCAGCCGTGCTGCAGAGCAGCGGCCTGTACTCTCT |
| | | GAGCAGCGTCGTGACAGTGCCCAGCAGCTCTCTGGGCACCCAGACCT |
| | | ACATCTGCAACGTGAACCACAAGCCCAGCAACACCAAGGTGGACAA |
| | | GAAGGTGGAACCCAAGAGCTGCGACAAGACCCACACCTGTCCCCCTT |
| | | GTCCTGCCCCCGAACTGCTGGGAGGCCCTTCCGTGTTCCTGTTCCCCC |
| | | CAAAGCCCAAGGACACCCTGATGATCAGCCGGACCCCCGAAGTGAC |
| | | CTGCGTGGTGGTGGATGTGTCCCACGAGGACCCTGAAGTGAAGTTCA |
| | | ATTGGTACGTGGACGGCGTGGAAGTGCACAACGCCAAGACCAAGCC |
| | | TAGAGAGGAACAGTACAACAGCACCTACCGGGTGGTGTCCGTGCTGA |
| | | CAGTGCTGCACCAGGACTGGCTGAACGGCAAAGAGTACAAGTGCAA |
| | | GGTGTCCAACAAGGCCCTGCCTGCCCCCATCGAGAAAACCATCAGCA |
| | | AGGCCAAGGGCCAGCCCCGCGAACCCCAGGTGTACACACTGCCCCCA |
| | | AGCAGGGACGAGCTGACCAAGAACCAGGTGTCCCTGACCTGTCTCGT |
| | | GAAAGGCTTCTACCCCTCCGATATCGCCGTGGAATGGGAGAGCAACG |
| | | GCCAGCCCGAGAACAACTACAAGACCACCCCCCCTGTGCTGGACAGC |
| | | GACGGCTCATTCTTCCTGTACAGCAAGCTGACCGTGGACAAGTCCCG |
| | | GTGGCAGCAGGGCAACGTGTTCAGCTGCAGCGTGATGCACGAGGCCC |
| | | TGCACAACCACTACACCCAGAAGTCCCTGAGCCTGAGCCCTGGCAAG |
| 440 | DNA | CAGTCTGTTCTGACACAGCCTCCATCTGTGTCTGGCGCCCCTGGACAG |
| | | AGAGTGACCATCAGCTGTACAGGCAGCAGCTCCAATATCGGAGCCGG |
| | | CTATGACGTGCACTGGTATCAGCAGCTGCCTGGCACAGCCCCTAAAC |
| | | TGCTGATCTACGGCAACAGCAACAGACCCAGCGGCGTGCCCGATAGA |
| | | TTTTCCGGCTCTAAGAGCGGCACAAGCGCCAGCCTGGCTATTACTGG |
| | | ACTGCAGGCCGAGGACGAGGCCGACTACTACTGTCAGTCTTACGCTG |
| | | GCCCCAATCCTTACGTGGTGTTTGGCGGCGGAACAAAGCTGACCGTT |
| | | CTAGGCCAGCCTAAAGCCGCCCCTAGCGTGACCCTGTTCCCTCCAAG |
| | | CAGCGAGGAACTGCAGGCCAACAAGGCCACCCTCGTGTGCCTGATCA |
| | | GCGACTTCTATCCTGGCGCCGTGACCGTGGCCTGGAAGGCCGATAGC |
| | | TCTCCTGTGAAGGCCGGCGTGGAAACCACCACCCCTAGCAAGCAGAG |
| | | CAACAACAAATACGCCGCCAGCAGCTACCTGAGCCTGACCCCCGAGC |
| | | AGTGGAAGTCCCACAGATCCTACAGCTGCCAAGTGACCCACGAGGGC |
| | | AGCACCGTGGAAAAGACAGTGGCCCCTACCGAGTGCAGC |
| 441 | PRT | EVQLLESGGGLVQPGGSLRLSCAASGFDFSSYEMNWVRQAPGKGLEWV |
| | | SGISWNSGWIDYADSVKGRFTISRDNSKNTLYLQMNSLRAEDTAVYYC |
| | | ARSGYSSSWFDPDFDYWGQGTLVTVSS |
| 442 | PRT | SYEMN |
| 443 | PRT | GISWNSGWIDYADSVKG |
| 444 | PRT | SGYSSSWFDPDFDY |
| 445 | PRT | QSVLTQPPSVSGAPGQRVTISCTGSSSNIGAGYDVHWYQQLPGTAPKLLI |
| | | YGNSNRPSGVPDRFSGSKSGTSASLAITGLQAEDEADYYCQSYAGPNPY |
| | | VVFGGGTKLTVL |
| 446 | PRT | TGSSSNIGAGYDVH |
| 447 | PRT | GNSNRPS |
| 448 | PRT | QSYAGPNPYVV |
| 449 | DNA | GAAGTTCAGCTGCTGGAATCTGGCGGCGGACTGGTTCAACCTGGCGG |
| | | ATCTCTGAGACTGAGCTGTGCCGCCAGCGGCTTCGATTTCAGCAGCT |
| | | ACGAGATGAACTGGGTCCGACAGGCCCCTGGCAAAGGCCTTGAATG |
| | | GGTGTCCGGCATCAGCTGGAATAGCGGCTGGATCGACTACGCCGACA |
| | | GCGTGAAGGGCAGATTCACCATCAGCCGGGACAACAGCAAGAACAC |
| | | CCTGTACCTGCAGATGAACAGCCTGAGAGCCGAGGACACCGCCGTGT |
| | | ACTACTGTGCCAGAAGCGGCTACAGCAGCTCTTGGTTTGACCCCGAC |
| | | TTCGACTATTGGGGCCAGGGCACACTGGTCACAGTCTCTTCA |
| 450 | DNA | AGCTACGAGATGAAC |
| 451 | DNA | GGCATCAGCTGGAATAGCGGCTGGATCGACTACGCCGACAGCGTGA |
| | | AGGGC |
| 452 | DNA | AGCGGCTACAGCAGCTCTTGGTTTGACCCCGACTTCGACTAT |
| 453 | DNA | CAGTCTGTTCTGACACAGCCTCCATCTGTGTCTGGCGCCCCTGGACAG |
| | | AGAGTGACCATCAGCTGTACAGGCAGCAGCTCCAATATCGGAGCCGG |
| | | CTATGACGTGCACTGGTATCAGCAGCTGCCTGGCACAGCCCCTAAAC |
| | | TGCTGATCTACGGCAACAGCAACAGACCCAGCGGCGTGCCCGATAGA |
| | | TTTTCCGGCTCTAAGAGCGGCACAAGCGCCAGCCTGGCTATTACTGG |
| | | ACTGCAGGCCGAGGACGAGGCCGACTACTACTGTCAGTCTTACGCTG |
| | | GCCCCAATCCTTACGTGGTGTTTGGCGGCGGAACAAAGCTGACCGTT |
| | | CTA |
| 454 | DNA | ACAGGCAGCAGCTCCAATATCGGAGCCGGCTATGACGTGCAC |
| 455 | DNA | GGCAACAGCAACAGACCCAGC |
| 456 | DNA | CAGTCTTACGCTGGCCCCAATCCTTACGTGGTG |
| 457 | PRT | EVQLLESGGGLVQPGGSLRLSCAASGFDFSSYEMNWVRQAPGKGLEWV |
| | | SGISWNSGWIDYADSVKGRFTISRDNSKNTLYLQMNSLRAEDTAVYYC |
| | | ARSGYSSSWFDPDFDYWGQGTLVTVSSASTKGPSVFPLAPSSKSTSGGT |
| | | AALGCLVKDYFPEPVTVSWNSGALTSGVHTFPAVLQSSGLYSLSSVVTV |
| | | PSSSLGTQTYICNVNHKPSNTKVDKKVEPKSCDKTHTCPPCPAPELLGGP |
| | | SVFLFPPKPKDTLMISRTPEVTCVVVDVSHEDPEVKFNWYVDGVEVHNA |
| | | KTKPREEQYNSTYRVVSVLTVLHQDWLNGKEYKCKVSNKALPAPIEKTI |
| | | SKAKGQPREPQVYTLPPSRDELTKNQVSLTCLVKGFYPSDIAVEWESNG |
| | | QPENNYKTTPPVLDSDGSFFLYSKLTVDKSRWQQGNVFSCSVMHEALH |
| | | NHYTQKSLSLSPGK |
| 458 | PRT | QSVLTQPPSVSGAPGQRVTISCTGSSSNIGAGYDVHWYQQLPGTAPKLLI |
| | | YGNSNRPSGVPDRFSGSKSGTSASLAITGLQAEDEADYYCQSYAGPNPY |
| | | VVFGGGTKLTVLGQPKAAPSVTLFPPSSEELQANKATLVCLISDFYPGAV |
| | | TVAWKADSSPVKAGVETTTPSKQSNNKYAASSYLSLTPEQWKSHRSYS |
| | | CQVTHEGSTVEKTVAPTECS |
| 459 | DNA | GAAGTTCAGCTGCTGGAATCTGGCGGCGGACTGGTTCAACCTGGCGG |
| | | ATCTCTGAGACTGAGCTGTGCCGCCAGCGGCTTCGATTTCAGCAGCT |
| | | ACGAGATGAACTGGGTCCGACAGGCCCCTGGCAAAGGCCTTGAATG |
| | | GGTGTCCGGCATCAGCTGGAATAGCGGCTGGATCGACTACGCCGACA |
| | | GCGTGAAGGGCAGATTCACCATCAGCCGGGACAACAGCAAGAACAC |
| | | CCTGTACCTGCAGATGAACAGCCTGAGAGCCGAGGACACCGCCGTGT |
| | | ACTACTGTGCCAGAAGCGGCTACAGCAGCTCTTGGTTTGACCCCGAC |
| | | TTCGACTATTGGGGCCAGGGCACACTGGTCACAGTCTCTTCAGCCAG |
| | | CACCAAGGGCCCCAGCGTGTTCCCTCTGGCCCCTAGCAGCAAGAGCA |
| | | CATCTGGCGGAACAGCCGCCCTGGGCTGCCTCGTGAAGGACTACTTT |
| | | CCCGAGCCCGTGACCGTGTCCTGGAACTCTGGCGCTCTGACAAGCGG |
| | | CGTGCACACCTTTCCAGCCGTGCTGCAGAGCAGCGGCCTGTACTCTCT |
| | | GAGCAGCGTCGTGACAGTGCCCAGCAGCTCTCTGGGCACCCAGACCT |
| | | ACATCTGCAACGTGAACCACAAGCCCAGCAACACCAAGGTGGACAA |
| | | GAAGGTGGAACCCAAGAGCTGCGACAAGACCCACACCTGTCCCCCTT |
| | | GTCCTGCCCCCGAACTGCTGGGAGGCCCTTCCGTGTTCCTGTTCCCCC |
| | | CAAAGCCCAAGGACACCCTGATGATCAGCCGGACCCCCGAAGTGAC |
| | | CTGCGTGGTGGTGGATGTGTCCCACGAGGACCCTGAAGTGAAGTTCA |
| | | ATTGGTACGTGGACGGCGTGGAAGTGCACAACGCCAAGACCAAGCC |
| | | TAGAGAGGAACAGTACAACAGCACCTACCGGGTGGTGTCCGTGCTGA |
| | | CAGTGCTGCACCAGGACTGGCTGAACGGCAAAGAGTACAAGTGCAA |
| | | GGTGTCCAACAAGGCCCTGCCTGCCCCCATCGAGAAAACCATCAGCA |
| | | AGGCCAAGGGCCAGCCCCGCGAACCCCAGGTGTACACACTGCCCCCA |
| | | AGCAGGGACGAGCTGACCAAGAACCAGGTGTCCCTGACCTGTCTCGT |
| | | GAAAGGCTTCTACCCCTCCGATATCGCCGTGGAATGGGAGAGCAACG |
| | | GCCAGCCCGAGAACAACTACAAGACCACCCCCCCTGTGCTGGACAGC |
| | | GACGGCTCATTCTTCCTGTACAGCAAGCTGACCGTGGACAAGTCCCG |
| | | GTGGCAGCAGGGCAACGTGTTCAGCTGCAGCGTGATGCACGAGGCCC |
| | | TGCACAACCACTACACCCAGAAGTCCCTGAGCCTGAGCCCTGGCAAG |
| 460 | DNA | CAGTCTGTTCTGACACAGCCTCCATCTGTGTCTGGCGCCCCTGGACAG |
| | | AGAGTGACCATCAGCTGTACAGGCAGCAGCTCCAATATCGGAGCCGG |
| | | CTATGACGTGCACTGGTATCAGCAGCTGCCTGGCACAGCCCCTAAAC |
| | | TGCTGATCTACGGCAACAGCAACAGACCCAGCGGCGTGCCCGATAGA |
| | | TTTTCCGGCTCTAAGAGCGGCACAAGCGCCAGCCTGGCTATTACTGG |
| | | ACTGCAGGCCGAGGACGAGGCCGACTACTACTGTCAGTCTTACGCTG |
| | | GCCCCAATCCTTACGTGGTGTTTGGCGGCGGAACAAAGCTGACCGTT |
| | | CTAGGCCAGCCTAAAGCCGCCCCTAGCGTGACCCTGTTCCCTCCAAG |
| | | CAGCGAGGAACTGCAGGCCAACAAGGCCACCCTCGTGTGCCTGATCA |
| | | GCGACTTCTATCCTGGCGCCGTGACCGTGGCCTGGAAGGCCGATAGC |
| | | TCTCCTGTGAAGGCCGGCGTGGAAACCACCACCCCTAGCAAGCAGAG |
| | | CAACAACAAATACGCCGCCAGCAGCTACCTGAGCCTGACCCCCGAGC |
| | | AGTGGAAGTCCCACAGATCCTACAGCTGCCAAGTGACCCACGAGGGC |
| | | AGCACCGTGGAAAAGACAGTGGCCCCTACCGAGTGCAGC |
| 461 | PRT | EVQLLESGGGLVQPGGSLRLSCAASGFTFDSYEMNWVRQAPGKGLEWV |
| | | SGISWNSGWIDYADSVKGRFTISRDNSKNTLYLQMNSLRAEDTAVYYC |
| | | ARSGYSSSWFDPDFDYWGQGTLVTVSS |
| 462 | PRT | SYEMN |
| 463 | PRT | GISWNSGWIDYADSVKG |
| 464 | PRT | SGYSSSWFDPDFDY |
| 465 | PRT | QSVLTQPPSVSGAPGQRVTISCTGSSSNIGAGYDVHWYQQLPGTAPKLLI |
| | | YGNSNRPSGVPDRFSGSKSGTSASLAITGLQAEDEADYYCQSYAGPNPY |
| | | VVFGGGTKLTVL |
| 466 | PRT | TGSSSNIGAGYDVH |
| 467 | PRT | GNSNRPS |
| 468 | PRT | QSYAGPNPYVV |
| 469 | DNA | GAAGTTCAGCTGCTGGAATCTGGCGGCGGACTGGTTCAACCTGGCGG |
| | | ATCTCTGAGACTGAGCTGTGCCGCCAGCGGCTTCACCTTCGATAGCT |
| | | ACGAGATGAACTGGGTCCGACAGGCCCCTGGCAAAGGCCTTGAATG |
| | | GGTGTCCGGCATCAGCTGGAATAGCGGCTGGATCGACTACGCCGACA |
| | | GCGTGAAGGGCAGATTCACCATCAGCCGGGACAACAGCAAGAACAC |
| | | CCTGTACCTGCAGATGAACAGCCTGAGAGCCGAGGACACCGCCGTGT |
| | | ACTACTGTGCCAGAAGCGGCTACAGCAGCTCTTGGTTTGACCCCGAC |
| | | TTCGACTATTGGGGCCAGGGCACACTGGTCACAGTCTCTTCA |
| 470 | DNA | AGCTACGAGATGAAC |
| 471 | DNA | GGCATCAGCTGGAATAGCGGCTGGATCGACTACGCCGACAGCGTGA |
| | | AGGGC |
| 472 | DNA | AGCGGCTACAGCAGCTCTTGGTTTGACCCCGACTTCGACTAT |
| 473 | DNA | CAGTCTGTTCTGACACAGCCTCCATCTGTGTCTGGCGCCCCTGGACAG |
| | | AGAGTGACCATCAGCTGTACAGGCAGCAGCTCCAATATCGGAGCCGG |
| | | CTATGACGTGCACTGGTATCAGCAGCTGCCTGGCACAGCCCCTAAAC |
| | | TGCTGATCTACGGCAACAGCAACAGACCCAGCGGCGTGCCCGATAGA |
| | | TTTTCCGGCTCTAAGAGCGGCACAAGCGCCAGCCTGGCTATTACTGG |
| | | ACTGCAGGCCGAGGACGAGGCCGACTACTACTGTCAGTCTTACGCTG |
| | | GCCCCAATCCTTACGTGGTGTTTGGCGGCGGAACAAAGCTGACCGTT |
| | | CTA |
| 474 | DNA | ACAGGCAGCAGCTCCAATATCGGAGCCGGCTATGACGTGCAC |
| 475 | DNA | GGCAACAGCAACAGACCCAGC |
| 476 | DNA | CAGTCTTACGCTGGCCCCAATCCTTACGTGGTG |
| 477 | PRT | EVQLLESGGGLVQPGGSLRLSCAASGFTFDSYEMNWVRQAPGKGLEWV |
| | | SGISWNSGWIDYADSVKGRFTISRDNSKNTLYLQMNSLRAEDTAVYYC |
| | | ARSGYSSSWFDPDFDYWGQGTLVTVSSASTKGPSVFPLAPSSKSTSGGT |
| | | AALGCLVKDYFPEPVTVSWNSGALTSGVHTFPAVLQSSGLYSLSSVVTV |
| | | PSSSLGTQTYICNVNHKPSNTKVDKKVEPKSCDKTHTCPPCPAPELLGGP |
| | | SVFLFPPKPKDTLMISRTPEVTCVVVDVSHEDPEVKFNWYVDGVEVHNA |
| | | KTKPREEQYNSTYRVVSVLTVLHQDWLNGKEYKCKVSNKALPAPIEKTI |
| | | SKAKGQPREPQVYTLPPSRDELTKNQVSLTCLVKGFYPSDIAVEWESNG |
| | | QPENNYKTTPPVLDSDGSFFLYSKLTVDKSRWQQGNVFSCSVMHEALH |
| | | NHYTQKSLSLSPGK |
| 478 | PRT | QSVLTQPPSVSGAPGQRVTISCTGSSSNIGAGYDVHWYQQLPGTAPKLLI |
| | | YGNSNRPSGVPDRFSGSKSGTSASLAITGLQAEDEADYYCQSYAGPNPY |
| | | VVFGGGTKLTVLGQPKAAPSVTLFPPSSEELQANKATLVCLISDFYPGAV |
| | | TVAWKADSSPVKAGVETTTPSKQSNNKYAASSYLSLTPEQWKSHRSYS |
| | | CQVTHEGSTVEKTVAPTECS |
| 479 | DNA | GAAGTTCAGCTGCTGGAATCTGGCGGCGGACTGGTTCAACCTGGCGG |
| | | ATCTCTGAGACTGAGCTGTGCCGCCAGCGGCTTCACCTTCGATAGCT |
| | | ACGAGATGAACTGGGTCCGACAGGCCCCTGGCAAAGGCCTTGAATG |
| | | GGTGTCCGGCATCAGCTGGAATAGCGGCTGGATCGACTACGCCGACA |
| | | GCGTGAAGGGCAGATTCACCATCAGCCGGGACAACAGCAAGAACAC |
| | | CCTGTACCTGCAGATGAACAGCCTGAGAGCCGAGGACACCGCCGTGT |
| | | ACTACTGTGCCAGAAGCGGCTACAGCAGCTCTTGGTTTGACCCCGAC |
| | | TTCGACTATTGGGGCCAGGGCACACTGGTCACAGTCTCTTCAGCCAG |
| | | CACCAAGGGCCCCAGCGTGTTCCCTCTGGCCCCTAGCAGCAAGAGCA |
| | | CATCTGGCGGAACAGCCGCCCTGGGCTGCCTCGTGAAGGACTACTTT |
| | | CCCGAGCCCGTGACCGTGTCCTGGAACTCTGGCGCTCTGACAAGCGG |
| | | CGTGCACACCTTTCCAGCCGTGCTGCAGAGCAGCGGCCTGTACTCTCT |
| | | GAGCAGCGTCGTGACAGTGCCCAGCAGCTCTCTGGGCACCCAGACCT |
| | | ACATCTGCAACGTGAACCACAAGCCCAGCAACACCAAGGTGGACAA |
| | | GAAGGTGGAACCCAAGAGCTGCGACAAGACCCACACCTGTCCCCCTT |
| | | GTCCTGCCCCCGAACTGCTGGGAGGCCCTTCCGTGTTCCTGTTCCCCC |
| | | CAAAGCCCAAGGACACCCTGATGATCAGCCGGACCCCCGAAGTGAC |
| | | CTGCGTGGTGGTGGATGTGTCCCACGAGGACCCTGAAGTGAAGTTCA |
| | | ATTGGTACGTGGACGGCGTGGAAGTGCACAACGCCAAGACCAAGCC |
| | | TAGAGAGGAACAGTACAACAGCACCTACCGGGTGGTGTCCGTGCTGA |
| | | CAGTGCTGCACCAGGACTGGCTGAACGGCAAAGAGTACAAGTGCAA |
| | | GGTGTCCAACAAGGCCCTGCCTGCCCCCATCGAGAAAACCATCAGCA |
| | | AGGCCAAGGGCCAGCCCCGCGAACCCCAGGTGTACACACTGCCCCCA |
| | | AGCAGGGACGAGCTGACCAAGAACCAGGTGTCCCTGACCTGTCTCGT |
| | | GAAAGGCTTCTACCCCTCCGATATCGCCGTGGAATGGGAGAGCAACG |
| | | GCCAGCCCGAGAACAACTACAAGACCACCCCCCCTGTGCTGGACAGC |
| | | GACGGCTCATTCTTCCTGTACAGCAAGCTGACCGTGGACAAGTCCCG |
| | | GTGGCAGCAGGGCAACGTGTTCAGCTGCAGCGTGATGCACGAGGCCC |
| | | TGCACAACCACTACACCCAGAAGTCCCTGAGCCTGAGCCCTGGCAAG |
| 480 | DNA | CAGTCTGTTCTGACACAGCCTCCATCTGTGTCTGGCGCCCCTGGACAG |
| | | AGAGTGACCATCAGCTGTACAGGCAGCAGCTCCAATATCGGAGCCGG |
| | | CTATGACGTGCACTGGTATCAGCAGCTGCCTGGCACAGCCCCTAAAC |
| | | TGCTGATCTACGGCAACAGCAACAGACCCAGCGGCGTGCCCGATAGA |
| | | TTTTCCGGCTCTAAGAGCGGCACAAGCGCCAGCCTGGCTATTACTGG |
| | | ACTGCAGGCCGAGGACGAGGCCGACTACTACTGTCAGTCTTACGCTG |
| | | GCCCCAATCCTTACGTGGTGTTTGGCGGCGGAACAAAGCTGACCGTT |
| | | CTAGGCCAGCCTAAAGCCGCCCCTAGCGTGACCCTGTTCCCTCCAAG |
| | | CAGCGAGGAACTGCAGGCCAACAAGGCCACCCTCGTGTGCCTGATCA |
| | | GCGACTTCTATCCTGGCGCCGTGACCGTGGCCTGGAAGGCCGATAGC |
| | | TCTCCTGTGAAGGCCGGCGTGGAAACCACCACCCCTAGCAAGCAGAG |
| | | CAACAACAAATACGCCGCCAGCAGCTACCTGAGCCTGACCCCCGAGC |
| | | AGTGGAAGTCCCACAGATCCTACAGCTGCCAAGTGACCCACGAGGGC |
| | | AGCACCGTGGAAAAGACAGTGGCCCCTACCGAGTGCAGC |
| 481 | PRT | EVQLLESGGGLVQPGGSLRLSCAASGFDFDSYEMNWVRQAPGKGLEW |
| | | VSGISWNSGWIDYADSVKGRFTISRDNSKNTLYLQMNSLRAEDTAVYY |
| | | CARSGYSSSWFDPDFDYWGQGTLVTVSS |
| 482 | PRT | SYEMN |
| 483 | PRT | GISWNSGWIDYADSVKG |
| 484 | PRT | SGYSSSWFDPDFDY |
| 485 | PRT | QSVLTQPPSVSGAPGQRVTISCTGSSSNIGAGYDVHWYQQLPGTAPKLLI |
| | | YGNSNRPSGVPDRFSGSKSGTSASLAITGLQAEDEADYYCQSYAGPNPY |
| | | VVFGGGTKLTVL |
| 486 | PRT | TGSSSNIGAGYDVH |
| 487 | PRT | GNSNRPS |
| 488 | PRT | QSYAGPNPYVV |
| 489 | DNA | GAAGTTCAGCTGCTGGAATCTGGCGGCGGACTGGTTCAACCTGGCGG |
| | | ATCTCTGAGACTGAGCTGTGCCGCCAGCGGCTTCGACTTCGATAGCT |
| | | ACGAGATGAACTGGGTCCGACAGGCCCCTGGCAAAGGCCTTGAATG |
| | | GGTGTCCGGCATCAGCTGGAATAGCGGCTGGATCGACTACGCCGACA |
| | | GCGTGAAGGGCAGATTCACCATCAGCCGGGACAACAGCAAGAACAC |
| | | CCTGTACCTGCAGATGAACAGCCTGAGAGCCGAGGACACCGCCGTGT |
| | | ACTACTGTGCCAGAAGCGGCTACAGCAGCTCTTGGTTTGACCCCGAC |
| | | TTCGACTATTGGGGCCAGGGCACACTGGTCACAGTCTCTTCA |
| 490 | DNA | AGCTACGAGATGAAC |
| 491 | DNA | GGCATCAGCTGGAATAGCGGCTGGATCGACTACGCCGACAGCGTGA |
| | | AGGGC |
| 492 | DNA | AGCGGCTACAGCAGCTCTTGGTTTGACCCCGACTTCGACTAT |
| 493 | DNA | CAGTCTGTTCTGACACAGCCTCCATCTGTGTCTGGCGCCCCTGGACAG |
| | | AGAGTGACCATCAGCTGTACAGGCAGCAGCTCCAATATCGGAGCCGG |
| | | CTATGACGTGCACTGGTATCAGCAGCTGCCTGGCACAGCCCCTAAAC |
| | | TGCTGATCTACGGCAACAGCAACAGACCCAGCGGCGTGCCCGATAGA |
| | | TTTTCCGGCTCTAAGAGCGGCACAAGCGCCAGCCTGGCTATTACTGG |
| | | ACTGCAGGCCGAGGACGAGGCCGACTACTACTGTCAGTCTTACGCTG |
| | | GCCCCAATCCTTACGTGGTGTTTGGCGGCGGAACAAAGCTGACCGTT |
| | | CTA |
| 494 | DNA | ACAGGCAGCAGCTCCAATATCGGAGCCGGCTATGACGTGCAC |
| 495 | DNA | GGCAACAGCAACAGACCCAGC |
| 496 | DNA | CAGTCTTACGCTGGCCCCAATCCTTACGTGGTG |
| 497 | PRT | EVQLLESGGGLVQPGGSLRLSCAASGFDFDSYEMNWVRQAPGKGLEW |
| | | VSGISWNSGWIDYADSVKGRFTISRDNSKNTLYLQMNSLRAEDTAVYY |
| | | CARSGYSSSWFDPDFDYWGQGTLVTVSSASTKGPSVFPLAPSSKSTSGG |
| | | TAALGCLVKDYFPEPVTVSWNSGALTSGVHTFPAVLQSSGLYSLSSVVT |
| | | VPSSSLGTQTYICNVNHKPSNTKVDKKVEPKSCDKTHTCPPCPAPELLGG |
| | | PSVFLFPPKPKDTLMISRTPEVTCVVVDVSHEDPEVKFNWYVDGVEVHN |
| | | AKTKPREEQYNSTYRVVSVLTVLHQDWLNGKEYKCKVSNKALPAPIEK |
| | | TISKAKGQPREPQVYTLPPSRDELTKNQVSLTCLVKGFYPSDIAVEWESN |
| | | GQPENNYKTTPPVLDSDGSFFLYSKLTVDKSRWQQGNVFSCSVMHEAL |
| | | HNHYTQKSLSLSPGK |
| 498 | PRT | QSVLTQPPSVSGAPGQRVTISCTGSSSNIGAGYDVHWYQQLPGTAPKLLI |
| | | YGNSNRPSGVPDRFSGSKSGTSASLAITGLQAEDEADYYCQSYAGPNPY |
| | | VVFGGGTKLTVLGQPKAAPSVTLFPPSSEELQANKATLVCLISDFYPGAV |
| | | TVAWKADSSPVKAGVETTTPSKQSNNKYAASSYLSLTPEQWKSHRSYS |
| | | CQVTHEGSTVEKTVAPTECS |
| 499 | DNA | GAAGTTCAGCTGCTGGAATCTGGCGGCGGACTGGTTCAACCTGGCGG |
| | | ATCTCTGAGACTGAGCTGTGCCGCCAGCGGCTTCGACTTCGATAGCT |
| | | ACGAGATGAACTGGGTCCGACAGGCCCCTGGCAAAGGCCTTGAATG |
| | | GGTGTCCGGCATCAGCTGGAATAGCGGCTGGATCGACTACGCCGACA |
| | | GCGTGAAGGGCAGATTCACCATCAGCCGGGACAACAGCAAGAACAC |
| | | CCTGTACCTGCAGATGAACAGCCTGAGAGCCGAGGACACCGCCGTGT |
| | | ACTACTGTGCCAGAAGCGGCTACAGCAGCTCTTGGTTTGACCCCGAC |
| | | TTCGACTATTGGGGCCAGGGCACACTGGTCACAGTCTCTTCAGCCAG |
| | | CACCAAGGGCCCCAGCGTGTTCCCTCTGGCCCCTAGCAGCAAGAGCA |
| | | CATCTGGCGGAACAGCCGCCCTGGGCTGCCTCGTGAAGGACTACTTT |
| | | CCCGAGCCCGTGACCGTGTCCTGGAACTCTGGCGCTCTGACAAGCGG |
| | | CGTGCACACCTTTCCAGCCGTGCTGCAGAGCAGCGGCCTGTACTCTCT |
| | | GAGCAGCGTCGTGACAGTGCCCAGCAGCTCTCTGGGCACCCAGACCT |
| | | ACATCTGCAACGTGAACCACAAGCCCAGCAACACCAAGGTGGACAA |
| | | GAAGGTGGAACCCAAGAGCTGCGACAAGACCCACACCTGTCCCCCTT |
| | | GTCCTGCCCCCGAACTGCTGGGAGGCCCTTCCGTGTTCCTGTTCCCCC |
| | | CAAAGCCCAAGGACACCCTGATGATCAGCCGGACCCCCGAAGTGAC |
| | | CTGCGTGGTGGTGGATGTGTCCCACGAGGACCCTGAAGTGAAGTTCA |
| | | ATTGGTACGTGGACGGCGTGGAAGTGCACAACGCCAAGACCAAGCC |
| | | TAGAGAGGAACAGTACAACAGCACCTACCGGGTGGTGTCCGTGCTGA |
| | | CAGTGCTGCACCAGGACTGGCTGAACGGCAAAGAGTACAAGTGCAA |
| | | GGTGTCCAACAAGGCCCTGCCTGCCCCCATCGAGAAAACCATCAGCA |
| | | AGGCCAAGGGCCAGCCCCGCGAACCCCAGGTGTACACACTGCCCCCA |
| | | AGCAGGGACGAGCTGACCAAGAACCAGGTGTCCCTGACCTGTCTCGT |
| | | GAAAGGCTTCTACCCCTCCGATATCGCCGTGGAATGGGAGAGCAACG |
| | | GCCAGCCCGAGAACAACTACAAGACCACCCCCCCTGTGCTGGACAGC |
| | | GACGGCTCATTCTTCCTGTACAGCAAGCTGACCGTGGACAAGTCCCG |
| | | GTGGCAGCAGGGCAACGTGTTCAGCTGCAGCGTGATGCACGAGGCCC |
| | | TGCACAACCACTACACCCAGAAGTCCCTGAGCCTGAGCCCTGGCAAG |
| 500 | DNA | CAGTCTGTTCTGACACAGCCTCCATCTGTGTCTGGCGCCCCTGGACAG |
| | | AGAGTGACCATCAGCTGTACAGGCAGCAGCTCCAATATCGGAGCCGG |
| | | CTATGACGTGCACTGGTATCAGCAGCTGCCTGGCACAGCCCCTAAAC |
| | | TGCTGATCTACGGCAACAGCAACAGACCCAGCGGCGTGCCCGATAGA |
| | | TTTTCCGGCTCTAAGAGCGGCACAAGCGCCAGCCTGGCTATTACTGG |
| | | ACTGCAGGCCGAGGACGAGGCCGACTACTACTGTCAGTCTTACGCTG |
| | | GCCCCAATCCTTACGTGGTGTTTGGCGGCGGAACAAAGCTGACCGTT |
| | | CTAGGCCAGCCTAAAGCCGCCCCTAGCGTGACCCTGTTCCCTCCAAG |
| | | CAGCGAGGAACTGCAGGCCAACAAGGCCACCCTCGTGTGCCTGATCA |
| | | GCGACTTCTATCCTGGCGCCGTGACCGTGGCCTGGAAGGCCGATAGC |
| | | TCTCCTGTGAAGGCCGGCGTGGAAACCACCACCCCTAGCAAGCAGAG |
| | | CAACAACAAATACGCCGCCAGCAGCTACCTGAGCCTGACCCCCGAGC |
| | | AGTGGAAGTCCCACAGATCCTACAGCTGCCAAGTGACCCACGAGGGC |
| | | AGCACCGTGGAAAAGACAGTGGCCCCTACCGAGTGCAGC |
| 501 | PRT | EVQLLESGGGLVQPGGSLRLSCAASGFTFDSYEMNWVRQAPGKGLEWV |
| | | SGISWNSGWIDYADSVKGRFTISRDNSKNTLYLQMNSLRAEDTAVYYC |
| | | ARSGYSSSWFDPDFDYWGQGTLVTVSS |
| 502 | PRT | SYEMN |
| 503 | PRT | GISWNSGWIDYADSVKG |
| 504 | PRT | SGYSSSWFDPDFDY |
| 505 | PRT | QSVLTQPPSVSGAPGQRVTISCTGSSSDIGAGYDVHWYQQLPGTAPKLLI |
| | | YGNSNRPSGVPDRFSGSKSGTSASLAITGLQAEDEADYYCQSYAGINPY |
| | | VVFGGGTKLTVL |
| 506 | PRT | TGSSSDIGAGYDVH |
| 507 | PRT | GNSNRPS |
| 508 | PRT | QSYAGINPYVV |
| 509 | DNA | GAAGTTCAGCTGCTGGAATCTGGCGGCGGACTGGTTCAACCTGGCGG |
| | | ATCTCTGAGACTGAGCTGTGCCGCCAGCGGCTTCACCTTCGATAGCT |
| | | ACGAGATGAACTGGGTCCGACAGGCCCCTGGCAAAGGCCTTGAATG |
| | | GGTGTCCGGCATCAGCTGGAATAGCGGCTGGATCGACTACGCCGACA |
| | | GCGTGAAGGGCAGATTCACCATCAGCCGGGACAACAGCAAGAACAC |
| | | CCTGTACCTGCAGATGAACAGCCTGAGAGCCGAGGACACCGCCGTGT |
| | | ACTACTGTGCCAGAAGCGGCTACAGCAGCTCTTGGTTTGACCCCGAC |
| | | TTCGACTATTGGGGCCAGGGCACACTGGTCACAGTCTCTTCA |
| 510 | DNA | AGCTACGAGATGAAC |
| 511 | DNA | GGCATCAGCTGGAATAGCGGCTGGATCGACTACGCCGACAGCGTGA |
| | | AGGGC |
| 512 | DNA | AGCGGCTACAGCAGCTCTTGGTTTGACCCCGACTTCGACTAT |
| 513 | DNA | CAGTCTGTTCTGACACAGCCTCCATCTGTGTCTGGCGCCCCTGGACAG |
| | | AGAGTGACCATCAGCTGTACAGGCAGCAGCTCCGATATTGGCGCCGG |
| | | ATACGACGTGCACTGGTATCAGCAACTGCCTGGCACAGCCCCTAAGC |
| | | TGCTGATCTACGGCAACAGCAACAGACCTAGCGGCGTGCCCGATAGA |
| | | TTCAGCGGCTCTAAGTCTGGCACAAGCGCCAGCCTGGCCATTACTGG |
| | | ACTGCAGGCCGAAGATGAGGCCGACTACTACTGTCAGAGCTACGCCG |
| | | GCATCAACCCCTACGTGGTGTTTGGCGGAGGCACCAAGCTGACAGTT |
| | | CTA |
| 514 | DNA | ACAGGCAGCAGCTCCGATATTGGCGCCGGATACGACGTGCAC |
| 515 | DNA | GGCAACAGCAACAGACCTAGC |
| 516 | DNA | CAGAGCTACGCCGGCATCAACCCCTACGTGGTG |
| 517 | PRT | EVQLLESGGGLVQPGGSLRLSCAASGFTFDSYEMNWVRQAPGKGLEWV |
| | | SGISWNSGWIDYADSVKGRFTISRDNSKNTLYLQMNSLRAEDTAVYYC |
| | | ARSGYSSSWFDPDFDYWGQGTLVTVSSASTKGPSVFPLAPSSKSTSGGT |
| | | AALGCLVKDYFPEPVTVSWNSGALTSGVHTFPAVLQSSGLYSLSSVVTV |
| | | PSSSLGTQTYICNVNHKPSNTKVDKKVEPKSCDKTHTCPPCPAPELLGGP |
| | | SVFLFPPKPKDTLMISRTPEVTCVVVDVSHEDPEVKFNWYVDGVEVHNA |
| | | KTKPREEQYNSTYRVVSVLTVLHQDWLNGKEYKCKVSNKALPAPIEKTI |
| | | SKAKGQPREPQVYTLPPSRDELTKNQVSLTCLVKGFYPSDIAVEWESNG |
| | | QPENNYKTTPPVLDSDGSFFLYSKLTVDKSRWQQGNVFSCSVMHEALH |
| | | NHYTQKSLSLSPGK |
| 518 | PRT | QSVLTQPPSVSGAPGQRVTISCTGSSSDIGAGYDVHWYQQLPGTAPKLLI |
| | | YGNSNRPSGVPDRFSGSKSGTSASLAITGLQAEDEADYYCQSYAGINPY |
| | | VVFGGGTKLTVLGQPKAAPSVTLFPPSSEELQANKATLVCLISDFYPGAV |
| | | TVAWKADSSPVKAGVETTTPSKQSNNKYAASSYLSLTPEQWKSHRSYS |
| | | CQVTHEGSTVEKTVAPTECS |
| 519 | DNA | GAAGTTCAGCTGCTGGAATCTGGCGGCGGACTGGTTCAACCTGGCGG |
| | | ATCTCTGAGACTGAGCTGTGCCGCCAGCGGCTTCACCTTCGATAGCT |
| | | ACGAGATGAACTGGGTCCGACAGGCCCCTGGCAAAGGCCTTGAATG |
| | | GGTGTCCGGCATCAGCTGGAATAGCGGCTGGATCGACTACGCCGACA |
| | | GCGTGAAGGGCAGATTCACCATCAGCCGGGACAACAGCAAGAACAC |
| | | CCTGTACCTGCAGATGAACAGCCTGAGAGCCGAGGACACCGCCGTGT |
| | | ACTACTGTGCCAGAAGCGGCTACAGCAGCTCTTGGTTTGACCCCGAC |
| | | TTCGACTATTGGGGCCAGGGCACACTGGTCACAGTCTCTTCAGCCAG |
| | | CACCAAGGGCCCCAGCGTGTTCCCTCTGGCCCCTAGCAGCAAGAGCA |
| | | CATCTGGCGGAACAGCCGCCCTGGGCTGCCTCGTGAAGGACTACTTT |
| | | CCCGAGCCCGTGACCGTGTCCTGGAACTCTGGCGCTCTGACAAGCGG |
| | | CGTGCACACCTTTCCAGCCGTGCTGCAGAGCAGCGGCCTGTACTCTCT |
| | | GAGCAGCGTCGTGACAGTGCCCAGCAGCTCTCTGGGCACCCAGACCT |
| | | ACATCTGCAACGTGAACCACAAGCCCAGCAACACCAAGGTGGACAA |
| | | GAAGGTGGAACCCAAGAGCTGCGACAAGACCCACACCTGTCCCCCTT |
| | | GTCCTGCCCCCGAACTGCTGGGAGGCCCTTCCGTGTTCCTGTTCCCCC |
| | | CAAAGCCCAAGGACACCCTGATGATCAGCCGGACCCCCGAAGTGAC |
| | | CTGCGTGGTGGTGGATGTGTCCCACGAGGACCCTGAAGTGAAGTTCA |
| | | ATTGGTACGTGGACGGCGTGGAAGTGCACAACGCCAAGACCAAGCC |
| | | TAGAGAGGAACAGTACAACAGCACCTACCGGGTGGTGTCCGTGCTGA |
| | | CAGTGCTGCACCAGGACTGGCTGAACGGCAAAGAGTACAAGTGCAA |
| | | GGTGTCCAACAAGGCCCTGCCTGCCCCCATCGAGAAAACCATCAGCA |
| | | AGGCCAAGGGCCAGCCCCGCGAACCCCAGGTGTACACACTGCCCCCA |
| | | AGCAGGGACGAGCTGACCAAGAACCAGGTGTCCCTGACCTGTCTCGT |
| | | GAAAGGCTTCTACCCCTCCGATATCGCCGTGGAATGGGAGAGCAACG |
| | | GCCAGCCCGAGAACAACTACAAGACCACCCCCCCTGTGCTGGACAGC |
| | | GACGGCTCATTCTTCCTGTACAGCAAGCTGACCGTGGACAAGTCCCG |
| | | GTGGCAGCAGGGCAACGTGTTCAGCTGCAGCGTGATGCACGAGGCCC |
| | | TGCACAACCACTACACCCAGAAGTCCCTGAGCCTGAGCCCTGGCAAG |
| 520 | DNA | CAGTCTGTTCTGACACAGCCTCCATCTGTGTCTGGCGCCCCTGGACAG |
| | | AGAGTGACCATCAGCTGTACAGGCAGCAGCTCCGATATTGGCGCCGG |
| | | ATACGACGTGCACTGGTATCAGCAACTGCCTGGCACAGCCCCTAAGC |
| | | TGCTGATCTACGGCAACAGCAACAGACCTAGCGGCGTGCCCGATAGA |
| | | TTCAGCGGCTCTAAGTCTGGCACAAGCGCCAGCCTGGCCATTACTGG |
| | | ACTGCAGGCCGAAGATGAGGCCGACTACTACTGTCAGAGCTACGCCG |
| | | GCATCAACCCCTACGTGGTGTTTGGCGGAGGCACCAAGCTGACAGTT |
| | | CTAGGCCAGCCTAAAGCCGCCCCTAGCGTGACCCTGTTCCCTCCAAG |
| | | CAGCGAGGAACTGCAGGCCAACAAGGCCACCCTCGTGTGCCTGATCA |
| | | GCGACTTCTATCCTGGCGCCGTGACCGTGGCCTGGAAGGCCGATAGC |
| | | TCTCCTGTGAAGGCCGGCGTGGAAACCACCACCCCTAGCAAGCAGAG |
| | | CAACAACAAATACGCCGCCAGCAGCTACCTGAGCCTGACCCCCGAGC |
| | | AGTGGAAGTCCCACAGATCCTACAGCTGCCAAGTGACCCACGAGGGC |
| | | AGCACCGTGGAAAAGACAGTGGCCCCTACCGAGTGCAGC |
| 521 | PRT | EVQLLESGGGLVQPGGSLRLSCAASGFDFDSYEMNWVRQAPGKGLEW |
| | | VSGISWNSGWIDYADSVKGRFTISRDNSKNTLYLQMNSLRAEDTAVYY |
| | | CARSGYSSSWFDPDFDYWGQGTLVTVSS |
| 522 | PRT | SYEMN |
| 523 | PRT | GISWNSGWIDYADSVKG |
| 524 | PRT | SGYSSSWFDPDFDY |
| 525 | PRT | QSVLTQPPSVSGAPGQRVTISCTGSSSDIGAGYDVHWYQQLPGTAPKLLI |
| | | YGNSNRPSGVPDRFSGSKSGTSASLAITGLQAEDEADYYCSSYEGINPYV |
| | | VFGGGTKLTVL |
| 526 | PRT | TGSSSDIGAGYDVH |
| 527 | PRT | GNSNRPS |
| 528 | PRT | SSYEGINPYVV |
| 529 | DNA | GAAGTTCAGCTGCTGGAATCTGGCGGCGGACTGGTTCAACCTGGCGG |
| | | ATCTCTGAGACTGAGCTGTGCCGCCAGCGGCTTCGACTTCGATAGCT |
| | | ACGAGATGAACTGGGTCCGACAGGCCCCTGGCAAAGGCCTTGAATG |
| | | GGTGTCCGGCATCAGCTGGAATAGCGGCTGGATCGACTACGCCGACA |
| | | GCGTGAAGGGCAGATTCACCATCAGCCGGGACAACAGCAAGAACAC |
| | | CCTGTACCTGCAGATGAACAGCCTGAGAGCCGAGGACACCGCCGTGT |
| | | ACTACTGTGCCAGAAGCGGCTACAGCAGCTCTTGGTTTGACCCCGAC |
| | | TTCGACTATTGGGGCCAGGGCACACTGGTCACAGTCTCTTCA |
| 530 | DNA | AGCTACGAGATGAAC |
| 531 | DNA | GGCATCAGCTGGAATAGCGGCTGGATCGACTACGCCGACAGCGTGA |
| | | AGGGC |
| 532 | DNA | AGCGGCTACAGCAGCTCTTGGTTTGACCCCGACTTCGACTAT |
| 533 | DNA | CAGTCTGTTCTGACACAGCCTCCATCTGTGTCTGGCGCCCCTGGACAG |
| | | AGAGTGACCATCAGCTGTACAGGCAGCAGCTCCGATATTGGCGCCGG |
| | | ATACGACGTGCACTGGTATCAGCAACTGCCTGGCACAGCCCCTAAGC |
| | | TGCTGATCTACGGCAACAGCAACAGACCTAGCGGCGTGCCCGATAGA |
| | | TTCAGCGGCTCTAAGTCTGGCACAAGCGCCAGCCTGGCCATTACTGG |
| | | ACTGCAGGCCGAAGATGAGGCCGACTACTACTGCAGCAGCTACGAG |
| | | GGCATCAACCCCTACGTGGTGTTTGGCGGCGGAACAAAGCTGACCGT |
| | | TCTA |
| 534 | DNA | ACAGGCAGCAGCTCCGATATTGGCGCCGGATACGACGTGCAC |
| 535 | DNA | GGCAACAGCAACAGACCTAGC |
| 536 | DNA | AGCAGCTACGAGGGCATCAACCCCTACGTGGTG |
| 537 | PRT | EVQLLESGGGLVQPGGSLRLSCAASGFDFDSYEMNWVRQAPGKGLEW |
| | | VSGISWNSGWIDYADSVKGRFTISRDNSKNTLYLQMNSLRAEDTAVYY |
| | | CARSGYSSSWFDPDFDYWGQGTLVTVSSASTKGPSVFPLAPSSKSTSGG |
| | | TAALGCLVKDYFPEPVTVSWNSGALTSGVHTFPAVLQSSGLYSLSSVVT |
| | | VPSSSLGTQTYICNVNHKPSNTKVDKKVEPKSCDKTHTCPPCPAPELLGG |
| | | PSVFLFPPKPKDTLMISRTPEVTCVVVDVSHEDPEVKFNWYVDGVEVHN |
| | | AKTKPREEQYNSTYRVVSVLTVLHQDWLNGKEYKCKVSNKALPAPIEK |
| | | TISKAKGQPREPQVYTLPPSRDELTKNQVSLTCLVKGFYPSDIAVEWESN |
| | | GQPENNYKTTPPVLDSDGSFFLYSKLTVDKSRWQQGNVFSCSVMHEAL |
| | | HNHYTQKSLSLSPGK |
| 538 | PRT | QSVLTQPPSVSGAPGQRVTISCTGSSSDIGAGYDVHWYQQLPGTAPKLLI |
| | | YGNSNRPSGVPDRFSGSKSGTSASLAITGLQAEDEADYYCSSYEGINPYV |
| | | VFGGGTKLTVLGQPKAAPSVTLFPPSSEELQANKATLVCLISDFYPGAVT |
| | | VAWKADSSPVKAGVETTTPSKQSNNKYAASSYLSLTPEQWKSHRSYSC |
| | | QVTHEGSTVEKTVAPTECS |
| 539 | DNA | GAAGTTCAGCTGCTGGAATCTGGCGGCGGACTGGTTCAACCTGGCGG |
| | | ATCTCTGAGACTGAGCTGTGCCGCCAGCGGCTTCGACTTCGATAGCT |
| | | ACGAGATGAACTGGGTCCGACAGGCCCCTGGCAAAGGCCTTGAATG |
| | | GGTGTCCGGCATCAGCTGGAATAGCGGCTGGATCGACTACGCCGACA |
| | | GCGTGAAGGGCAGATTCACCATCAGCCGGGACAACAGCAAGAACAC |
| | | CCTGTACCTGCAGATGAACAGCCTGAGAGCCGAGGACACCGCCGTGT |
| | | ACTACTGTGCCAGAAGCGGCTACAGCAGCTCTTGGTTTGACCCCGAC |
| | | TTCGACTATTGGGGCCAGGGCACACTGGTCACAGTCTCTTCAGCCAG |
| | | CACCAAGGGCCCCAGCGTGTTCCCTCTGGCCCCTAGCAGCAAGAGCA |
| | | CATCTGGCGGAACAGCCGCCCTGGGCTGCCTCGTGAAGGACTACTTT |
| | | CCCGAGCCCGTGACCGTGTCCTGGAACTCTGGCGCTCTGACAAGCGG |
| | | CGTGCACACCTTTCCAGCCGTGCTGCAGAGCAGCGGCCTGTACTCTCT |
| | | GAGCAGCGTCGTGACAGTGCCCAGCAGCTCTCTGGGCACCCAGACCT |
| | | ACATCTGCAACGTGAACCACAAGCCCAGCAACACCAAGGTGGACAA |
| | | GAAGGTGGAACCCAAGAGCTGCGACAAGACCCACACCTGTCCCCCTT |
| | | GTCCTGCCCCCGAACTGCTGGGAGGCCCTTCCGTGTTCCTGTTCCCCC |
| | | CAAAGCCCAAGGACACCCTGATGATCAGCCGGACCCCCGAAGTGAC |
| | | CTGCGTGGTGGTGGATGTGTCCCACGAGGACCCTGAAGTGAAGTTCA |
| | | ATTGGTACGTGGACGGCGTGGAAGTGCACAACGCCAAGACCAAGCC |
| | | TAGAGAGGAACAGTACAACAGCACCTACCGGGTGGTGTCCGTGCTGA |
| | | CAGTGCTGCACCAGGACTGGCTGAACGGCAAAGAGTACAAGTGCAA |
| | | GGTGTCCAACAAGGCCCTGCCTGCCCCCATCGAGAAAACCATCAGCA |
| | | AGGCCAAGGGCCAGCCCCGCGAACCCCAGGTGTACACACTGCCCCCA |
| | | AGCAGGGACGAGCTGACCAAGAACCAGGTGTCCCTGACCTGTCTCGT |
| | | GAAAGGCTTCTACCCCTCCGATATCGCCGTGGAATGGGAGAGCAACG |
| | | GCCAGCCCGAGAACAACTACAAGACCACCCCCCCTGTGCTGGACAGC |
| | | GACGGCTCATTCTTCCTGTACAGCAAGCTGACCGTGGACAAGTCCCG |
| | | GTGGCAGCAGGGCAACGTGTTCAGCTGCAGCGTGATGCACGAGGCCC |
| | | TGCACAACCACTACACCCAGAAGTCCCTGAGCCTGAGCCCTGGCAAG |
| 540 | DNA | CAGTCTGTTCTGACACAGCCTCCATCTGTGTCTGGCGCCCCTGGACAG |
| | | AGAGTGACCATCAGCTGTACAGGCAGCAGCTCCGATATTGGCGCCGG |
| | | ATACGACGTGCACTGGTATCAGCAACTGCCTGGCACAGCCCCTAAGC |
| | | TGCTGATCTACGGCAACAGCAACAGACCTAGCGGCGTGCCCGATAGA |
| | | TTCAGCGGCTCTAAGTCTGGCACAAGCGCCAGCCTGGCCATTACTGG |
| | | ACTGCAGGCCGAAGATGAGGCCGACTACTACTGCAGCAGCTACGAG |
| | | GGCATCAACCCCTACGTGGTGTTTGGCGGCGGAACAAAGCTGACCGT |
| | | TCTAGGCCAGCCTAAAGCCGCCCCTAGCGTGACCCTGTTCCCTCCAA |
| | | GCAGCGAGGAACTGCAGGCCAACAAGGCCACCCTCGTGTGCCTGATC |
| | | AGCGACTTCTATCCTGGCGCCGTGACCGTGGCCTGGAAGGCCGATAG |
| | | CTCTCCTGTGAAGGCCGGCGTGGAAACCACCACCCCTAGCAAGCAGA |
| | | GCAACAACAAATACGCCGCCAGCAGCTACCTGAGCCTGACCCCCGAG |
| | | CAGTGGAAGTCCCACAGATCCTACAGCTGCCAAGTGACCCACGAGGG |
| | | CAGCACCGTGGAAAAGACAGTGGCCCCTACCGAGTGCAGC |
| 541 | PRT | EVQLLESGGGLVQPGGSLRLSCAASGFDFSSYEMNWVRQAPGKGLEWV |
| | | SGISWNSGWIDYADSVKGRFTISRDNSKNTLYLQMNSLRAEDTAVYYC |
| | | ARSGYSSSWFDPDFDYWGQGTLVTVSS |
| 542 | PRT | SYEMN |
| 543 | PRT | GISWNSGWIDYADSVKG |
| 544 | PRT | SGYSSSWFDPDFDY |
| 545 | PRT | QSVLTQPPSVSGAPGQRVTISCTGSSSNIGAGYDVHWYQQLPGTAPKLLI |
| | | YGASNRPSGVPDRFSGSKSGTSASLAITGLQAEDEADYYCSSYEGPNPYV |
| | | VFGGGTKLTVL |
| 546 | PRT | TGSSSNIGAGYDVH |
| 547 | PRT | GASNRPS |
| 548 | PRT | SSYEGPNPYVV |
| 549 | DNA | GAAGTTCAGCTGCTGGAATCTGGCGGCGGACTGGTTCAACCTGGCGG |
| | | ATCTCTGAGACTGAGCTGTGCCGCCAGCGGCTTCGATTTCAGCAGCT |
| | | ACGAGATGAACTGGGTCCGACAGGCCCCTGGCAAAGGCCTTGAATG |
| | | GGTGTCCGGCATCAGCTGGAATAGCGGCTGGATCGACTACGCCGACA |
| | | GCGTGAAGGGCAGATTCACCATCAGCCGGGACAACAGCAAGAACAC |
| | | CCTGTACCTGCAGATGAACAGCCTGAGAGCCGAGGACACCGCCGTGT |
| | | ACTACTGTGCCAGAAGCGGCTACAGCAGCTCTTGGTTTGACCCCGAC |
| | | TTCGACTATTGGGGCCAGGGCACACTGGTCACAGTCTCTTCA |
| 550 | DNA | AGCTACGAGATGAAC |
| 551 | DNA | GGCATCAGCTGGAATAGCGGCTGGATCGACTACGCCGACAGCGTGA |
| | | AGGGC |
| 552 | DNA | AGCGGCTACAGCAGCTCTTGGTTTGACCCCGACTTCGACTAT |
| 553 | DNA | CAGTCTGTTCTGACACAGCCTCCATCTGTGTCTGGCGCCCCTGGACAG |
| | | AGAGTGACCATCAGCTGTACAGGCAGCAGCTCCAATATCGGAGCCGG |
| | | CTATGACGTGCACTGGTATCAGCAGCTGCCTGGCACAGCCCCTAAAC |
| | | TGCTGATCTACGGCGCCAGCAATAGACCTAGCGGCGTGCCCGATAGA |
| | | TTCAGCGGCTCTAAGTCTGGCACAAGCGCCAGCCTGGCCATTACTGG |
| | | ACTGCAGGCCGAAGATGAGGCCGACTACTACTGCAGCAGCTACGAG |
| | | GGCCCCAATCCTTACGTGGTGTTTGGCGGCGGAACAAAGCTGACCGT |
| | | TCTA |
| 554 | DNA | ACAGGCAGCAGCTCCAATATCGGAGCCGGCTATGACGTGCAC |
| 555 | DNA | GGCGCCAGCAATAGACCTAGC |
| 556 | DNA | AGCAGCTACGAGGGCCCCAATCCTTACGTGGTG |
| 557 | PRT | EVQLLESGGGLVQPGGSLRLSCAASGFDFSSYEMNWVRQAPGKGLEWV |
| | | SGISWNSGWIDYADSVKGRFTISRDNSKNTLYLQMNSLRAEDTAVYYC |
| | | ARSGYSSSWFDPDFDYWGQGTLVTVSSASTKGPSVFPLAPSSKSTSGGT |
| | | AALGCLVKDYFPEPVTVSWNSGALTSGVHTFPAVLQSSGLYSLSSVVTV |
| | | PSSSLGTQTYICNVNHKPSNTKVDKKVEPKSCDKTHTCPPCPAPELLGGP |
| | | SVFLFPPKPKDTLMISRTPEVTCVVVDVSHEDPEVKFNWYVDGVEVHNA |
| | | KTKPREEQYNSTYRVVSVLTVLHQDWLNGKEYKCKVSNKALPAPIEKTI |
| | | SKAKGQPREPQVYTLPPSRDELTKNQVSLTCLVKGFYPSDIAVEWESNG |
| | | QPENNYKTTPPVLDSDGSFFLYSKLTVDKSRWQQGNVFSCSVMHEALH |
| | | NHYTQKSLSLSPGK |
| 558 | PRT | QSVLTQPPSVSGAPGQRVTISCTGSSSNIGAGYDVHWYQQLPGTAPKLLI |
| | | YGASNRPSGVPDRFSGSKSGTSASLAITGLQAEDEADYYCSSYEGPNPYV |
| | | VFGGGTKLTVLGQPKAAPSVTLFPPSSEELQANKATLVCLISDFYPGAVT |
| | | VAWKADSSPVKAGVETTTPSKQSNNKYAASSYLSLTPEQWKSHRSYSC |
| | | QVTHEGSTVEKTVAPTECS |
| 559 | DNA | GAAGTTCAGCTGCTGGAATCTGGCGGCGGACTGGTTCAACCTGGCGG |
| | | ATCTCTGAGACTGAGCTGTGCCGCCAGCGGCTTCGATTTCAGCAGCT |
| | | ACGAGATGAACTGGGTCCGACAGGCCCCTGGCAAAGGCCTTGAATG |
| | | GGTGTCCGGCATCAGCTGGAATAGCGGCTGGATCGACTACGCCGACA |
| | | GCGTGAAGGGCAGATTCACCATCAGCCGGGACAACAGCAAGAACAC |
| | | CCTGTACCTGCAGATGAACAGCCTGAGAGCCGAGGACACCGCCGTGT |
| | | ACTACTGTGCCAGAAGCGGCTACAGCAGCTCTTGGTTTGACCCCGAC |
| | | TTCGACTATTGGGGCCAGGGCACACTGGTCACAGTCTCTTCAGCCAG |
| | | CACCAAGGGCCCCAGCGTGTTCCCTCTGGCCCCTAGCAGCAAGAGCA |
| | | CATCTGGCGGAACAGCCGCCCTGGGCTGCCTCGTGAAGGACTACTTT |
| | | CCCGAGCCCGTGACCGTGTCCTGGAACTCTGGCGCTCTGACAAGCGG |
| | | CGTGCACACCTTTCCAGCCGTGCTGCAGAGCAGCGGCCTGTACTCTCT |
| | | GAGCAGCGTCGTGACAGTGCCCAGCAGCTCTCTGGGCACCCAGACCT |
| | | ACATCTGCAACGTGAACCACAAGCCCAGCAACACCAAGGTGGACAA |
| | | GAAGGTGGAACCCAAGAGCTGCGACAAGACCCACACCTGTCCCCCTT |
| | | GTCCTGCCCCCGAACTGCTGGGAGGCCCTTCCGTGTTCCTGTTCCCCC |
| | | CAAAGCCCAAGGACACCCTGATGATCAGCCGGACCCCCGAAGTGAC |
| | | CTGCGTGGTGGTGGATGTGTCCCACGAGGACCCTGAAGTGAAGTTCA |
| | | ATTGGTACGTGGACGGCGTGGAAGTGCACAACGCCAAGACCAAGCC |
| | | TAGAGAGGAACAGTACAACAGCACCTACCGGGTGGTGTCCGTGCTGA |
| | | CAGTGCTGCACCAGGACTGGCTGAACGGCAAAGAGTACAAGTGCAA |
| | | GGTGTCCAACAAGGCCCTGCCTGCCCCCATCGAGAAAACCATCAGCA |
| | | AGGCCAAGGGCCAGCCCCGCGAACCCCAGGTGTACACACTGCCCCCA |
| | | AGCAGGGACGAGCTGACCAAGAACCAGGTGTCCCTGACCTGTCTCGT |
| | | GAAAGGCTTCTACCCCTCCGATATCGCCGTGGAATGGGAGAGCAACG |
| | | GCCAGCCCGAGAACAACTACAAGACCACCCCCCCTGTGCTGGACAGC |
| | | GACGGCTCATTCTTCCTGTACAGCAAGCTGACCGTGGACAAGTCCCG |
| | | GTGGCAGCAGGGCAACGTGTTCAGCTGCAGCGTGATGCACGAGGCCC |
| | | TGCACAACCACTACACCCAGAAGTCCCTGAGCCTGAGCCCTGGCAAG |
| 560 | DNA | CAGTCTGTTCTGACACAGCCTCCATCTGTGTCTGGCGCCCCTGGACAG |
| | | AGAGTGACCATCAGCTGTACAGGCAGCAGCTCCAATATCGGAGCCGG |
| | | CTATGACGTGCACTGGTATCAGCAGCTGCCTGGCACAGCCCCTAAAC |
| | | TGCTGATCTACGGCGCCAGCAATAGACCTAGCGGCGTGCCCGATAGA |
| | | TTCAGCGGCTCTAAGTCTGGCACAAGCGCCAGCCTGGCCATTACTGG |
| | | ACTGCAGGCCGAAGATGAGGCCGACTACTACTGCAGCAGCTACGAG |
| | | GGCCCCAATCCTTACGTGGTGTTTGGCGGCGGAACAAAGCTGACCGT |
| | | TCTAGGCCAGCCTAAAGCCGCCCCTAGCGTGACCCTGTTCCCTCCAA |
| | | GCAGCGAGGAACTGCAGGCCAACAAGGCCACCCTCGTGTGCCTGATC |
| | | AGCGACTTCTATCCTGGCGCCGTGACCGTGGCCTGGAAGGCCGATAG |
| | | CTCTCCTGTGAAGGCCGGCGTGGAAACCACCACCCCTAGCAAGCAGA |
| | | GCAACAACAAATACGCCGCCAGCAGCTACCTGAGCCTGACCCCCGAG |
| | | CAGTGGAAGTCCCACAGATCCTACAGCTGCCAAGTGACCCACGAGGG |
| | | CAGCACCGTGGAAAAGACAGTGGCCCCTACCGAGTGCAGC |
| 561 | PRT | EVQLLESGGGLVQPGGSLRLSCAASGFTFDSYEMNWVRQAPGKGLEWV |
| | | SGISWNSGWIDYADSVKGRFTISRDNSKNTLYLQMNSLRAEDTAVYYC |
| | | ARSGYSSSWFDPDFDYWGQGTLVTVSS |
| 562 | PRT | SYEMN |
| 563 | PRT | GISWNSGWIDYADSVKG |
| 564 | PRT | SGYSSSWFDPDFDY |
| 565 | PRT | QSVLTQPPSVSGAPGQRVTISCTGSSSNIGAGYDVHWYQQLPGTAPKLLI |
| | | YGNSNRPSGVPDRFSGSKSGTSASLAITGLQAEDEADYYCSSYAGPNPY |
| | | VVFGGGTKLTVL |
| 566 | PRT | TGSSSNIGAGYDVH |
| 567 | PRT | GNSNRPS |
| 568 | PRT | SSYAGPNPYVV |
| 569 | DNA | GAAGTTCAGCTGCTGGAATCTGGCGGCGGACTGGTTCAACCTGGCGG |
| | | ATCTCTGAGACTGAGCTGTGCCGCCAGCGGCTTCACCTTCGATAGCT |
| | | ACGAGATGAACTGGGTCCGACAGGCCCCTGGCAAAGGCCTTGAATG |
| | | GGTGTCCGGCATCAGCTGGAATAGCGGCTGGATCGACTACGCCGACA |
| | | GCGTGAAGGGCAGATTCACCATCAGCCGGGACAACAGCAAGAACAC |
| | | CCTGTACCTGCAGATGAACAGCCTGAGAGCCGAGGACACCGCCGTGT |
| | | ACTACTGTGCCAGAAGCGGCTACAGCAGCTCTTGGTTTGACCCCGAC |
| | | TTCGACTATTGGGGCCAGGGCACACTGGTCACAGTCTCTTCA |
| 570 | DNA | AGCTACGAGATGAAC |
| 571 | DNA | GGCATCAGCTGGAATAGCGGCTGGATCGACTACGCCGACAGCGTGA |
| | | AGGGC |
| 572 | DNA | AGCGGCTACAGCAGCTCTTGGTTTGACCCCGACTTCGACTAT |
| 573 | DNA | CAGTCTGTTCTGACACAGCCTCCATCTGTGTCTGGCGCCCCTGGACAG |
| | | AGAGTGACCATCAGCTGTACAGGCAGCAGCTCCAATATCGGAGCCGG |
| | | CTATGACGTGCACTGGTATCAGCAGCTGCCTGGCACAGCCCCTAAAC |
| | | TGCTGATCTACGGCAACAGCAACAGACCCAGCGGCGTGCCCGATAGA |
| | | TTTTCCGGCTCTAAGAGCGGCACAAGCGCCAGCCTGGCTATTACTGG |
| | | ACTGCAGGCCGAGGACGAGGCCGACTACTACTGTAGCTCTTACGCTG |
| | | GCCCCAATCCTTACGTGGTGTTTGGCGGCGGAACAAAGCTGACCGTT |
| | | CTA |
| 574 | DNA | ACAGGCAGCAGCTCCAATATCGGAGCCGGCTATGACGTGCAC |
| 575 | DNA | GGCAACAGCAACAGACCCAGC |
| 576 | DNA | AGCTCTTACGCTGGCCCCAATCCTTACGTGGTG |
| 577 | PRT | EVQLLESGGGLVQPGGSLRLSCAASGFTFDSYEMNWVRQAPGKGLEWV |
| | | SGISWNSGWIDYADSVKGRFTISRDNSKNTLYLQMNSLRAEDTAVYYC |
| | | ARSGYSSSWFDPDFDYWGQGTLVTVSSASTKGPSVFPLAPSSKSTSGGT |
| | | AALGCLVKDYFPEPVTVSWNSGALTSGVHTFPAVLQSSGLYSLSSVVTV |
| | | PSSSLGTQTYICNVNHKPSNTKVDKKVEPKSCDKTHTCPPCPAPELLGGP |
| | | SVFLFPPKPKDTLMISRTPEVTCVVVDVSHEDPEVKFNWYVDGVEVHNA |
| | | KTKPREEQYNSTYRVVSVLTVLHQDWLNGKEYKCKVSNKALPAPIEKTI |
| | | SKAKGQPREPQVYTLPPSRDELTKNQVSLTCLVKGFYPSDIAVEWESNG |
| | | QPENNYKTTPPVLDSDGSFFLYSKLTVDKSRWQQGNVFSCSVMHEALH |
| | | NHYTQKSLSLSPGK |
| 578 | PRT | QSVLTQPPSVSGAPGQRVTISCTGSSSNIGAGYDVHWYQQLPGTAPKLLI |
| | | YGNSNRPSGVPDRFSGSKSGTSASLAITGLQAEDEADYYCSSYAGPNPY |
| | | VVFGGGTKLTVLGQPKAAPSVTLFPPSSEELQANKATLVCLISDFYPGAV |
| | | TVAWKADSSPVKAGVETTTPSKQSNNKYAASSYLSLTPEQWKSHRSYS |
| | | CQVTHEGSTVEKTVAPTECS |
| 579 | DNA | GAAGTTCAGCTGCTGGAATCTGGCGGCGGACTGGTTCAACCTGGCGG |
| | | ATCTCTGAGACTGAGCTGTGCCGCCAGCGGCTTCACCTTCGATAGCT |
| | | ACGAGATGAACTGGGTCCGACAGGCCCCTGGCAAAGGCCTTGAATG |
| | | GGTGTCCGGCATCAGCTGGAATAGCGGCTGGATCGACTACGCCGACA |
| | | GCGTGAAGGGCAGATTCACCATCAGCCGGGACAACAGCAAGAACAC |
| | | CCTGTACCTGCAGATGAACAGCCTGAGAGCCGAGGACACCGCCGTGT |
| | | ACTACTGTGCCAGAAGCGGCTACAGCAGCTCTTGGTTTGACCCCGAC |
| | | TTCGACTATTGGGGCCAGGGCACACTGGTCACAGTCTCTTCAGCCAG |
| | | CACCAAGGGCCCCAGCGTGTTCCCTCTGGCCCCTAGCAGCAAGAGCA |
| | | CATCTGGCGGAACAGCCGCCCTGGGCTGCCTCGTGAAGGACTACTTT |
| | | CCCGAGCCCGTGACCGTGTCCTGGAACTCTGGCGCTCTGACAAGCGG |
| | | CGTGCACACCTTTCCAGCCGTGCTGCAGAGCAGCGGCCTGTACTCTCT |
| | | GAGCAGCGTCGTGACAGTGCCCAGCAGCTCTCTGGGCACCCAGACCT |
| | | ACATCTGCAACGTGAACCACAAGCCCAGCAACACCAAGGTGGACAA |
| | | GAAGGTGGAACCCAAGAGCTGCGACAAGACCCACACCTGTCCCCCTT |
| | | GTCCTGCCCCCGAACTGCTGGGAGGCCCTTCCGTGTTCCTGTTCCCCC |
| | | CAAAGCCCAAGGACACCCTGATGATCAGCCGGACCCCCGAAGTGAC |
| | | CTGCGTGGTGGTGGATGTGTCCCACGAGGACCCTGAAGTGAAGTTCA |
| | | ATTGGTACGTGGACGGCGTGGAAGTGCACAACGCCAAGACCAAGCC |
| | | TAGAGAGGAACAGTACAACAGCACCTACCGGGTGGTGTCCGTGCTGA |
| | | CAGTGCTGCACCAGGACTGGCTGAACGGCAAAGAGTACAAGTGCAA |
| | | GGTGTCCAACAAGGCCCTGCCTGCCCCCATCGAGAAAACCATCAGCA |
| | | AGGCCAAGGGCCAGCCCCGCGAACCCCAGGTGTACACACTGCCCCCA |
| | | AGCAGGGACGAGCTGACCAAGAACCAGGTGTCCCTGACCTGTCTCGT |
| | | GAAAGGCTTCTACCCCTCCGATATCGCCGTGGAATGGGAGAGCAACG |
| | | GCCAGCCCGAGAACAACTACAAGACCACCCCCCCTGTGCTGGACAGC |
| | | GACGGCTCATTCTTCCTGTACAGCAAGCTGACCGTGGACAAGTCCCG |
| | | GTGGCAGCAGGGCAACGTGTTCAGCTGCAGCGTGATGCACGAGGCCC |
| | | TGCACAACCACTACACCCAGAAGTCCCTGAGCCTGAGCCCTGGCAAG |
| 580 | DNA | CAGTCTGTTCTGACACAGCCTCCATCTGTGTCTGGCGCCCCTGGACAG |
| | | AGAGTGACCATCAGCTGTACAGGCAGCAGCTCCAATATCGGAGCCGG |
| | | CTATGACGTGCACTGGTATCAGCAGCTGCCTGGCACAGCCCCTAAAC |
| | | TGCTGATCTACGGCAACAGCAACAGACCCAGCGGCGTGCCCGATAGA |
| | | TTTTCCGGCTCTAAGAGCGGCACAAGCGCCAGCCTGGCTATTACTGG |
| | | ACTGCAGGCCGAGGACGAGGCCGACTACTACTGTAGCTCTTACGCTG |
| | | GCCCCAATCCTTACGTGGTGTTTGGCGGCGGAACAAAGCTGACCGTT |
| | | CTAGGCCAGCCTAAAGCCGCCCCTAGCGTGACCCTGTTCCCTCCAAG |
| | | CAGCGAGGAACTGCAGGCCAACAAGGCCACCCTCGTGTGCCTGATCA |
| | | GCGACTTCTATCCTGGCGCCGTGACCGTGGCCTGGAAGGCCGATAGC |
| | | TCTCCTGTGAAGGCCGGCGTGGAAACCACCACCCCTAGCAAGCAGAG |
| | | CAACAACAAATACGCCGCCAGCAGCTACCTGAGCCTGACCCCCGAGC |
| | | AGTGGAAGTCCCACAGATCCTACAGCTGCCAAGTGACCCACGAGGGC |
| | | AGCACCGTGGAAAAGACAGTGGCCCCTACCGAGTGCAGC |
| 581 | PRT | HHHHHHKNNVPRLKLSYKEMLESNNVITFNGLANSSSYHTFLLDEERSR |
| | | LYVGAKDHIFSFDLVNIKDFQKIVWPVSYTRRDECKWAGKDILKECANF |
| | | IKVLKAYNQTHLYACGTGAFHPICTYIEIGHHPEDNIFKLENSHFENGRG |
| | | KSPYDPKLLTASLLIDGELYSGTAADFMGRDFAIFRTLGHHHPIRTEQHD |
| | | SRWLNDPKFISAHLISESDNPEDDKVYFFFRENAIDGEHSGKATHARIGQI |
| | | CKNDFGGHRSLVNKWTTFLKARLICSVPGPNGIDTHFDELQDVFLMNFK |
| | | DPKNPVVYGVFTTSSNIFKGSAVCMYSMSDVRRVFLGPYAHRDGPNYQ |
| | | WVPYQGRVPYPRPGTCPSKTFGGFDSTKDLPDDVITFARSHPAMYNPVF |
| | | PMNNRPIVIKTDVNYQFTQIVVDRVDAEDGQYDVMFIGTDVGTVLKVV |
| | | SIPKETWYDLEEVLLEEMTVFREPTAISAMELSTKQQQLYIGSTAGVAQL |
| | | PLHRCDIYGKACAECCLARDPYCAWDGSACSRYFPTAKRATRAQDIRN |
| | | GDPLTHCSDLHHDNHHGHSPEERIIYGVENSSTFLECSPKSQRALVYWQF |
| | | QRRNEERKEEIRVDDHIIRTDQGLLLRSLQQKDSGNYLCHAVEHGFIQTL |
| | | LKVTLEVIDTEHLEELLHKDDDGDGSKTKEMSNSMTPSQKVWYRDFMQ |
| | | LINHPNLNTMDEFCEQVWKRDRKQRRQRPGHTPGNSNKWKHLQENKK |
| | | GRNRRTHEFERAPRSVDIEGRMDPKSCDKTHTCPPCPAPELLGGPSVFLF |
| | | PPKPKDTLMISRTPEVTCVVVDVSHEDPEVKFNWYVDGVEVHNAKTKP |
| | | REEQYNSTYRVVSVLTVLHQDWLNGKEYKCKVSNKALPAPIEKTISKAK |
| | | GQPREPQVYTLPPSRDELTKNQVSLTCLVKGFYPSDIAVEWESNGQPEN |
| | | NYKTTPPVLDSDGSFFLYSKLTVDKSRWQQGNVFSCSVMHEALHNHYT |
| | | QKSLSLSPGK |
| 582 | PRT | NYQNGKNNVPRLKLSYKEMLESNNVITFNGLANSSSYHTFLLDEERSRL |
| | | YVGAKDHIFSFDLVNIKDFQKIVWPVSYTRRDECKWAGKDILKECANFI |
| | | KVLKAYNQTHLYACGTGAFHPICTYIEIGHHPEDNIFKLENSHFENGRGK |
| | | SPYDPKLLTASLLIDGELYSGTAADFMGRDFAIFRTLGHHHPIRTEQHDS |
| | | RWLNDPKFISAHLISESDNPEDDKVYFFFRENAIDGEHSGKATHARIGQIC |
| | | KNDFGGHRSLVNKWTTFLKARLICSVPGPNGIDTHFDELQDVFLMNFKD |
| | | PKNPVVYGVFTTSSNIFKGSAVCMYSMSDVRRVFLGPYAHRDGPNYQW |
| | | VPYQGRVPYPRPGTCPSKTFGGFDSTKDLPDDVITFARSHPAMYNPVFP |
| | | MNNRPIVIKTDVNYQFTQIVVDRVDAEDGQYDVMFIGTDVGTVLKVVSI |
| | | PKETWYDLEEVLLEEMTVFREPTAISAMELSTKQQQLYIGSTAGVAQLP |
| | | LHRCDIYGKACAECCLARDPYCAWDGSACSRYFPTAKARTRAQDIRNG |
| | | DPLTHCSDGGIEGRMDHHHHHH |
| 583 | PRT | NYANGKNNVPRLKLSYKEMLESNNVITFNGLANSSSYHTFLLDEERSRL |
| | | YVGAKDHIFSFNLVNIKDFQKIVWPVSYTRRDECKWAGKDILKECANFI |
| | | KVLEAYNQTHLYACGTGAFHPICTYIEVGHHPEDNIFKLQDSHFENGRG |
| | | KSPYDPKLLTASLLIDGELYSGTAADFMGRDFAIFRTLGHHHPIRTEQHD |
| | | SRWLNDPRFISAHLIPESDNPEDDKVYFFFRENAIDGEHSGKATHARIGQI |
| | | CKNDFGGHRSLVNKWTTFLKARLICSVPGPNGIDTHFDELQDVFLMNSK |
| | | DPKNPIVYGVFTTSSNIFKGSAVCMYSMSDVRRVFLGPYAHRDGPNYQ |
| | | WVPYQGRVPYPRPGTCPSKTFGGFDSTKDLPDDVITFARSHPAMYNPVF |
| | | PINNRPIMIKTDVNYQFTQIVVDRVDAEDGQYDVMFIGTDVGTVLKVVS |
| | | VPKETWHDLEEILLEEMTVFREPTTISAMELSTKQQQLYIGSTAGVAQLP |
| | | LHRCDIYGKACAECCLARDPYCAWDGSSCSRYFPTAKARTRAQDIRNG |
| | | DPLTHCSDGGIEGRMDHHHHHH |
| 584 | PRT | NYANGKNNVPRLKLSYKEMLESNNVITFNGLANSSSYHTFLLDEERSRL |
| | | YVGAKDHIFSFNLVNIKDFQKIVWPVSYTRRDECKWAGKDILKECANFI |
| | | KVLKAYNQTHLYACGTGAFHPICTYIEVGHHPEDNIFKLQDSHFENGRG |
| | | KSPYDPKLLTASLLIDGELYSGTAADFMGRDFAIFRTLGHHHPIRTEQHD |
| | | SRWLNDPRFISAHLIPESDNPEDDKVYFFFRENAIDGEHSGKATHARIGQI |
| | | CKNDFGGHRSLVNKWTTFLKARLICSVPGPNGIDTHFDELQDVFLMNSK |
| | | DPKNPIVYGVFTTSSNIFKGSAVCMYSMSDVRRVFLGPYAHRDGPNYQ |
| | | WVPYQGRVPYPRPGTCPSKTFGGFDSTKDLPDDVITFARSHPAMYNPVF |
| | | PINNRPIMIKTDVNYQFTQIVVDRVDAEDGQYDVMFIGTDVGTVLKVVS |
| | | VPKETWHDLEEVLLEEMTVFREPTTISAMELSTKQQQLYIGSTAGVAQL |
| | | PLHRCDIYGKACAECCLARDPYCAWDGSSCSRYFPTAKARTRAQDIRNG |
| | | DPLTHCSDGGIEGRMDHHHHHH |
| 585 | PRT | NYQNGKNNVPRLKLSYKEMLESNSVITFNGLANSSSYHTFLLDEERSRL |
| | | YVGAKDHIFSFNLVNIKDFQKIVWPVSYTRRDECKWAGKDIQKECANFI |
| | | KVLKAYNQTHLYACGTGAFHPICTYIEIGHHPEDNIFKLEDSHFENGRGK |
| | | SPYDPKLLTASLLIDGELYSGTAADFMGRDFAIFRTLGHHHPIRTEQHDS |
| | | RWLNDPRFISAHLIPESDNPEDDKVYFFFRENAIDGEHTGKATHARIGQIC |
| | | KNDFGGHRSLVNKWTTFLKARLICSVPGPNGIDTHFDELQDVFLMNSKD |
| | | PKNPIVYGVFTTSSNIFKGSAVCMYSMSDVRRVFLGPYAHRDGPNYQW |
| | | VPYQGRVPYPRPGTCPSKTFGGFDSTKDLPDDVITFARSHPAMYNPVFPI |
| | | NNRPIMIKTDVNYQFTQIVVDRVDAEDGQYDVMFIGTDVGTVLKVVSIP |
| | | KETWHDLEEVLLEEMTVFREPTPISAMELSTKQHQLYAGSPAGLAQLPL |
| | | QRCAAYGRACAECCLARDPYCAWDGAACSRYFPAAKARTRAQDIRNG |
| | | DPLTHCSDGGIEGRMDHHHHHH |
| 586 | PRT | NYQNGKNNVPRLKLSYKEMLESNNVITFNGLANSSSYHTFLLDEERSRL |
| | | YVGAKDHIFSFNLVNIKDFQKIVWPVSYTRRDECKWAGKDILKECANFI |
| | | KVLKAYNQTHLYACGTGAFHPICTYIEIGHHPEDNIFKLENSHFENGRGK |
| | | SPYDPKLLTASLLIDGELYSGTAADFMGRDFAIFRTLGHHHPIRTEQHDS |
| | | RWLNDPRFISAHLIPESDNPEDDKVYFFFRENAIDGEHSGKATHARIGQIC |
| | | KNDFGGHRSLVNKWTTFLKARLICSVPGPNGIDTHFDELQDVFLMNFKD |
| | | PKNPIVYGVFTTSSNIFKGSAVCMYSMSDVRRVFLGPYAHRDGPNYQW |
| | | VPYQGRVPYPRPGTCPSKTFGGFDSTKDLPDDVITFARSHPAMYNPVFPI |
| | | NNRPIMIKTDVNYQFTQIVVDRVDAEDGQYDVMFIGTDVGTVLKVVSIP |
| | | KETWHDLEEVLLEEMTVFREPTTISAMELSTKQQQLYIGSTAGIAQLPLH |
| | | RCDIYGKACAECCLARDPYCAWDGSSCSRYFPTAKARTRAQDIRNGDPL |
| | | THCSDGGIEGRMDHHHHHH |
| 587 | PRT | NYQNGKNNVPRLKLSYKEMLESNNVITFNGLANSSSYHTFLLDEERSRL |
| | | YVGAKDHIFSFNLVNIKDFQKIVWPVSYTRRDECKWAGKDILKECANFI |
| | | KVLKAYNQTHLYACGTGAFHPICTYIEIGHHPEDNIFKLEDSHFENGRGK |
| | | SPYDPKLLTASLLIDGELYSGTAADFMGRDFAIFRTLGHHHPIRTEQHDS |
| | | RWLNDPRFISAHLIPESDNPEDDKVYFFFRENAIDGEHTGKATHARIGQIC |
| | | KNDFGGHRSLVNKWTTFLKARLICSVPGPNGIDTHFDELQDVFLMNSKD |
| | | PKNPVVYGVFTTSSNIFKGSAVCMYSMSDVRRVFLGPYAHRDGPNYQW |
| | | VPYQGRVPYPRPGTCPSKTFGGFDSTKDLPDDVITFARSHPAMYNPVFPI |
| | | NNRPIMIKTDVNYQFTQIVVDRVDAEDGQYDVMFIGTDVGTVLKVVSIP |
| | | KETWHDLEEVLLEEMTVFREPTTISAMELSTKQQQLYVGSAAGVAQLPL |
| | | HRCDIYGKACAECCLARDPYCAWDGSSCSRYFPTAKARTRAQDIRNGD |
| | | PLTHCSDGGIEGRMDHHHHHH |
| 800 | PRT | EVQLVESGGGLVQPGGSLRLSCAASGFTFSSYYMSWVRQAPGKGLEWV |
| | | STIIKSGGYAYYPDSVKDRFTISRDNSKNTLYLQMSSLRAEDTAVYYCVR |
| | | GGQGAMDYWGQGTTVTVSS |
| 801 | PRT | SYYMS |
| 802 | PRT | TIIKSGGYAYYPDSVKD |
| 803 | PRT | GGQGAMDY |
| 804 | PRT | EIVLTQSPATLSLSPGERATLSCRASQSIGDYLHWYQQKPGQAPRLLIKY |
| | | ASQSISGIPARFSGSGSGTDFTLTITSLEPEDFAVYYCQQGYSFPYTFGGG |
| | | TKLEIK |
| 805 | PRT | RASQSIGDYLH |
| 806 | PRT | YASQSIS |
| 807 | PRT | QQGYSFPYT |
| 808 | DNA | GAAGTGCAGCTGGTGGAATCTGGCGGAGGACTGGTTCAACCTGGCGG |
| | | CTCTCTGAGACTGTCTTGTGCCGCCAGCGGCTTCACCTTCAGCAGCTA |
| | | CTACATGAGCTGGGTCCGACAGGCCCCTGGCAAAGGACTTGAATGGG |
| | | TGTCCACCATCATCAAGAGCGGCGGCTACGCCTACTATCCCGACAGC |
| | | GTGAAGGACCGGTTCACCATCTCCAGAGACAACAGCAAGAACACCCT |
| | | GTACCTGCAGATGAGCAGCCTGAGAGCCGAGGATACCGCCGTGTACT |
| | | ACTGTGTTAGAGGCGGACAGGGCGCCATGGATTATTGGGGCCAGGG |
| | | AACCACAGTGACCGTGTCATCA |
| 809 | DNA | GAGATTGTGCTGACACAGTCTCCCGCCACACTGTCTCTTAGCCCTGGC |
| | | GAAAGAGCCACACTGAGCTGTAGAGCCAGCCAGAGCATCGGCGATT |
| | | ACCTGCACTGGTATCAGCAGAAGCCTGGACAGGCCCCTCGGCTGCTG |
| | | ATTAAGTACGCCAGCCAGTCCATCAGCGGCATCCCTGCCAGATTTTCT |
| | | GGCAGCGGCTCTGGCACCGATTTCACCCTGACCATCACCAGCCTGGA |
| | | ACCTGAGGACTTCGCCGTGTACTACTGCCAGCAGGGCTACAGCTTCC |
| | | CCTACACATTTGGCGGAGGCACCAAGCTGGAAATCAAA |
| 810 | PRT | EVQLVESGGGLVQPGGSLRLSCAASGFTFSSYYMSWVRQAPGKGLEWV |
| | | STIIKSGGYAYYPDSVKDRFTISRDNSKNTLYLQMSSLRAEDTAVYYCVR |
| | | GGQGAMDYWGQGTTVTVSSASTKGPSVFPLAPSSKSTSGGTAALGCLV |
| | | KDYFPEPVTVSWNSGALTSGVHTFPAVLQSSGLYSLSSVVTVPSSSLGTQ |
| | | TYICNVNHKPSNTKVDKKVEPKSCDKTHTCPPCPAPELLGGPSVFLFPPK |
| | | PKDTLMISRTPEVTCVVVDVSHEDPEVKFNWYVDGVEVHNAKTKPREE |
| | | QYNSTYRVVSVLTVLHQDWLNGKEYKCKVSNKALPAPIEKTISKAKGQ |
| | | PREPQVYTLPPSRDELTKNQVSLTCLVKGFYPSDIAVEWESNGQPENNY |
| | | KTTPPVLDSDGSFFLYSKLTVDKSRWQQGNVFSCSVMHEALHNHYTQK |
| | | SLSLSPGK |
| 811 | PRT | EIVLTQSPATLSLSPGERATLSCRASQSIGDYLHWYQQKPGQAPRLLIKY |
| | | ASQSISGIPARFSGSGSGTDFTLTITSLEPEDFAVYYCQQGYSFPYTFGGG |
| | | TKLEIKRTVAAPSVFIFPPSDEQLKSGTASVVCLLNNFYPREAKVQWKVD |
| | | NALQSGNSQESVTEQDSKDSTYSLSSTLTLSKADYEKHKVYACEVTHQG |
| | | LSSPVTKSFNRGEC |
| 812 | DNA | GAAGTGCAGCTGGTGGAATCTGGCGGAGGACTGGTTCAACCTGGCGG |
| | | CTCTCTGAGACTGTCTTGTGCCGCCAGCGGCTTCACCTTCAGCAGCTA |
| | | CTACATGAGCTGGGTCCGACAGGCCCCTGGCAAAGGACTTGAATGGG |
| | | TGTCCACCATCATCAAGAGCGGCGGCTACGCCTACTATCCCGACAGC |
| | | GTGAAGGACCGGTTCACCATCTCCAGAGACAACAGCAAGAACACCCT |
| | | GTACCTGCAGATGAGCAGCCTGAGAGCCGAGGATACCGCCGTGTACT |
| | | ACTGTGTTAGAGGCGGACAGGGCGCCATGGATTATTGGGGCCAGGG |
| | | AACCACAGTGACCGTGTCATCAGCCAGCACCAAGGGCCCCAGCGTGT |
| | | TCCCTCTGGCCCCTAGCAGCAAGAGCACATCTGGCGGAACAGCCGCC |
| | | CTGGGCTGCCTCGTGAAGGACTACTTTCCCGAGCCCGTGACCGTGTC |
| | | CTGGAACTCTGGCGCTCTGACAAGCGGCGTGCACACCTTTCCAGCCG |
| | | TGCTGCAGAGCAGCGGCCTGTACTCTCTGAGCAGCGTCGTGACAGTG |
| | | CCCAGCAGCTCTCTGGGCACCCAGACCTACATCTGCAACGTGAACCA |
| | | CAAGCCCAGCAACACCAAGGTGGACAAGAAGGTGGAACCCAAGAGC |
| | | TGCGACAAGACCCACACCTGTCCCCCTTGTCCTGCCCCCGAACTGCTG |
| | | GGAGGCCCTTCCGTGTTCCTGTTCCCCCCAAAGCCCAAGGACACCCT |
| | | GATGATCAGCCGGACCCCCGAAGTGACCTGCGTGGTGGTGGATGTGT |
| | | CCCACGAGGACCCTGAAGTGAAGTTCAATTGGTACGTGGACGGCGTG |
| | | GAAGTGCACAACGCCAAGACCAAGCCTAGAGAGGAACAGTACAACA |
| | | GCACCTACCGGGTGGTGTCCGTGCTGACAGTGCTGCACCAGGACTGG |
| | | CTGAACGGCAAAGAGTACAAGTGCAAGGTGTCCAACAAGGCCCTGC |
| | | CTGCCCCCATCGAGAAAACCATCAGCAAGGCCAAGGGCCAGCCCCG |
| | | CGAACCCCAGGTGTACACACTGCCCCCAAGCAGGGACGAGCTGACC |
| | | AAGAACCAGGTGTCCCTGACCTGTCTCGTGAAAGGCTTCTACCCCTC |
| | | CGATATCGCCGTGGAATGGGAGAGCAACGGCCAGCCCGAGAACAAC |
| | | TACAAGACCACCCCCCCTGTGCTGGACAGCGACGGCTCATTCTTCCT |
| | | GTACAGCAAGCTGACCGTGGACAAGTCCCGGTGGCAGCAGGGCAAC |
| | | GTGTTCAGCTGCAGCGTGATGCACGAGGCCCTGCACAACCACTACAC |
| | | CCAGAAGTCCCTGAGCCTGAGCCCTGGCAAG |
| 813 | DNA | GAGATTGTGCTGACACAGTCTCCCGCCACACTGTCTCTTAGCCCTGGC |
| | | GAAAGAGCCACACTGAGCTGTAGAGCCAGCCAGAGCATCGGCGATT |
| | | ACCTGCACTGGTATCAGCAGAAGCCTGGACAGGCCCCTCGGCTGCTG |
| | | ATTAAGTACGCCAGCCAGTCCATCAGCGGCATCCCTGCCAGATTTTCT |
| | | GGCAGCGGCTCTGGCACCGATTTCACCCTGACCATCACCAGCCTGGA |
| | | ACCTGAGGACTTCGCCGTGTACTACTGCCAGCAGGGCTACAGCTTCC |
| | | CCTACACATTTGGCGGAGGCACCAAGCTGGAAATCAAACGAACCGTG |
| | | GCCGCTCCCAGCGTGTTCATCTTCCCACCTAGCGACGAGCAGCTGAA |
| | | GTCCGGCACAGCCTCTGTCGTGTGCCTGCTGAACAACTTCTACCCCCG |
| | | CGAGGCCAAGGTGCAGTGGAAGGTGGACAATGCCCTGCAGAGCGGC |
| | | AACAGCCAGGAAAGCGTGACCGAGCAGGACAGCAAGGACTCCACCT |
| | | ACAGCCTGAGCAGCACCCTGACCCTGAGCAAGGCCGACTACGAGAA |
| | | GCACAAGGTGTACGCCTGCGAAGTGACCCACCAGGGCCTGTCTAGCC |
| | | CCGTGACCAAGAGCTTCAACCGGGGCGAGTGT |
| 814 | PRT | EVQLVESGGGLVQPGGSLRLSCAASGFPFSSYYMSWVRQAPGKGLEWV |
| | | STIIKSGGYAYYPDSVKDRFTISRDNSKNTLYLQMSSLRAEDTAVYYCVR |
| | | GGQGAMDYWGQGTTVTVSS |
| 815 | PRT | SYYMS |
| 816 | PRT | TIIKSGGYAYYPDSVKD |
| 817 | PRT | GGQGAMDY |
| 818 | PRT | EIVLTQSPATLSLSPGERATLSCRASQSIGDYLHWYQQKPGQAPRLLIKY |
| | | ASQSISGIPARFSGSGSGTDFTLTITSLEPEDFAVYYCQQGYSFPYTFGGG |
| | | TKLEIK |
| 819 | PRT | RASQSIGDYLH |
| 820 | PRT | YASQSIS |
| 821 | PRT | QQGYSFPYT |
| 822 | DNA | GAAGTGCAGCTGGTGGAATCTGGCGGAGGACTGGTTCAACCTGGCGG |
| | | CTCTCTGAGACTGTCTTGTGCCGCCTCTGGCTTCCCATTCAGCAGCTA |
| | | CTACATGAGCTGGGTCCGACAGGCCCCTGGCAAAGGACTTGAATGGG |
| | | TGTCCACCATCATCAAGAGCGGCGGCTACGCCTACTATCCCGACAGC |
| | | GTGAAGGACCGGTTCACCATCAGCCGGGACAACAGCAAGAACACCC |
| | | TGTACCTGCAGATGAGCAGCCTGAGAGCCGAGGATACCGCCGTGTAC |
| | | TACTGTGTTAGAGGCGGACAGGGCGCCATGGATTATTGGGGCCAGGG |
| | | AACCACAGTGACCGTGTCATCA |
| 823 | DNA | GAGATTGTGCTGACACAGTCTCCCGCCACACTGTCTCTTAGCCCTGGC |
| | | GAAAGAGCCACACTGAGCTGTAGAGCCAGCCAGAGCATCGGCGATT |
| | | ACCTGCACTGGTATCAGCAGAAGCCTGGACAGGCCCCTCGGCTGCTG |
| | | ATTAAGTACGCCAGCCAGTCCATCAGCGGCATCCCTGCCAGATTTTCT |
| | | GGCAGCGGCTCTGGCACCGATTTCACCCTGACCATCACCAGCCTGGA |
| | | ACCTGAGGACTTCGCCGTGTACTACTGCCAGCAGGGCTACAGCTTCC |
| | | CCTACACATTTGGCGGAGGCACCAAGCTGGAAATCAAA |
| 824 | PRT | EVQLVESGGGLVQPGGSLRLSCAASGFPFSSYYMSWVRQAPGKGLEWV |
| | | STIIKSGGYAYYPDSVKDRFTISRDNSKNTLYLQMSSLRAEDTAVYYCVR |
| | | GGQGAMDYWGQGTTVTVSSASTKGPSVFPLAPSSKSTSGGTAALGCLV |
| | | KDYFPEPVTVSWNSGALTSGVHTFPAVLQSSGLYSLSSVVTVPSSSLGTQ |
| | | TYICNVNHKPSNTKVDKKVEPKSCDKTHTCPPCPAPELLGGPSVFLFPPK |
| | | PKDTLMISRTPEVTCVVVDVSHEDPEVKFNWYVDGVEVHNAKTKPREE |
| | | QYNSTYRVVSVLTVLHQDWLNGKEYKCKVSNKALPAPIEKTISKAKGQ |
| | | PREPQVYTLPPSRDELTKNQVSLTCLVKGFYPSDIAVEWESNGQPENNY |
| | | KTTPPVLDSDGSFFLYSKLTVDKSRWQQGNVFSCSVMHEALHNHYTQK |
| | | SLSLSPGK |
| 825 | PRT | EIVLTQSPATLSLSPGERATLSCRASQSIGDYLHWYQQKPGQAPRLLIKY |
| | | ASQSISGIPARFSGSGSGTDFTLTITSLEPEDFAVYYCQQGYSFPYTFGGG |
| | | TKLEIKRTVAAPSVFIFPPSDEQLKSGTASVVCLLNNFYPREAKVQWKVD |
| | | NALQSGNSQESVTEQDSKDSTYSLSSTLTLSKADYEKHKVYACEVTHQG |
| | | LSSPVTKSFNRGEC |
| 826 | DNA | GAAGTGCAGCTGGTGGAATCTGGCGGAGGACTGGTTCAACCTGGCGG |
| | | CTCTCTGAGACTGTCTTGTGCCGCCTCTGGCTTCCCATTCAGCAGCTA |
| | | CTACATGAGCTGGGTCCGACAGGCCCCTGGCAAAGGACTTGAATGGG |
| | | TGTCCACCATCATCAAGAGCGGCGGCTACGCCTACTATCCCGACAGC |
| | | GTGAAGGACCGGTTCACCATCAGCCGGGACAACAGCAAGAACACCC |
| | | TGTACCTGCAGATGAGCAGCCTGAGAGCCGAGGATACCGCCGTGTAC |
| | | TACTGTGTTAGAGGCGGACAGGGCGCCATGGATTATTGGGGCCAGGG |
| | | AACCACAGTGACCGTGTCATCAGCCAGCACCAAGGGCCCCAGCGTGT |
| | | TCCCTCTGGCCCCTAGCAGCAAGAGCACATCTGGCGGAACAGCCGCC |
| | | CTGGGCTGCCTCGTGAAGGACTACTTTCCCGAGCCCGTGACCGTGTC |
| | | CTGGAACTCTGGCGCTCTGACAAGCGGCGTGCACACCTTTCCAGCCG |
| | | TGCTGCAGAGCAGCGGCCTGTACTCTCTGAGCAGCGTCGTGACAGTG |
| | | CCCAGCAGCTCTCTGGGCACCCAGACCTACATCTGCAACGTGAACCA |
| | | CAAGCCCAGCAACACCAAGGTGGACAAGAAGGTGGAACCCAAGAGC |
| | | TGCGACAAGACCCACACCTGTCCCCCTTGTCCTGCCCCCGAACTGCTG |
| | | GGAGGCCCTTCCGTGTTCCTGTTCCCCCCAAAGCCCAAGGACACCCT |
| | | GATGATCAGCCGGACCCCCGAAGTGACCTGCGTGGTGGTGGATGTGT |
| | | CCCACGAGGACCCTGAAGTGAAGTTCAATTGGTACGTGGACGGCGTG |
| | | GAAGTGCACAACGCCAAGACCAAGCCTAGAGAGGAACAGTACAACA |
| | | GCACCTACCGGGTGGTGTCCGTGCTGACAGTGCTGCACCAGGACTGG |
| | | CTGAACGGCAAAGAGTACAAGTGCAAGGTGTCCAACAAGGCCCTGC |
| | | CTGCCCCCATCGAGAAAACCATCAGCAAGGCCAAGGGCCAGCCCCG |
| | | CGAACCCCAGGTGTACACACTGCCCCCAAGCAGGGACGAGCTGACC |
| | | AAGAACCAGGTGTCCCTGACCTGTCTCGTGAAAGGCTTCTACCCCTC |
| | | CGATATCGCCGTGGAATGGGAGAGCAACGGCCAGCCCGAGAACAAC |
| | | TACAAGACCACCCCCCCTGTGCTGGACAGCGACGGCTCATTCTTCCT |
| | | GTACAGCAAGCTGACCGTGGACAAGTCCCGGTGGCAGCAGGGCAAC |
| | | GTGTTCAGCTGCAGCGTGATGCACGAGGCCCTGCACAACCACTACAC |
| | | CCAGAAGTCCCTGAGCCTGAGCCCTGGCAAG |
| 827 | DNA | GAGATTGTGCTGACACAGTCTCCCGCCACACTGTCTCTTAGCCCTGGC |
| | | GAAAGAGCCACACTGAGCTGTAGAGCCAGCCAGAGCATCGGCGATT |
| | | ACCTGCACTGGTATCAGCAGAAGCCTGGACAGGCCCCTCGGCTGCTG |
| | | ATTAAGTACGCCAGCCAGTCCATCAGCGGCATCCCTGCCAGATTTTCT |
| | | GGCAGCGGCTCTGGCACCGATTTCACCCTGACCATCACCAGCCTGGA |
| | | ACCTGAGGACTTCGCCGTGTACTACTGCCAGCAGGGCTACAGCTTCC |
| | | CCTACACATTTGGCGGAGGCACCAAGCTGGAAATCAAACGAACCGTG |
| | | GCCGCTCCCAGCGTGTTCATCTTCCCACCTAGCGACGAGCAGCTGAA |
| | | GTCCGGCACAGCCTCTGTCGTGTGCCTGCTGAACAACTTCTACCCCCG |
| | | CGAGGCCAAGGTGCAGTGGAAGGTGGACAATGCCCTGCAGAGCGGC |
| | | AACAGCCAGGAAAGCGTGACCGAGCAGGACAGCAAGGACTCCACCT |
| | | ACAGCCTGAGCAGCACCCTGACCCTGAGCAAGGCCGACTACGAGAA |
| | | GCACAAGGTGTACGCCTGCGAAGTGACCCACCAGGGCCTGTCTAGCC |
| | | CCGTGACCAAGAGCTTCAACCGGGGCGAGTGT |
| 828 | PRT | EVQLVESGGGLVQLGGSLRLSCAASGFTFSSYYMSWVRQAPGKGLEWV |
| | | STIIKSGGYAYYPDSVKDRFTISRDNSKNTLYLQMNSLRAEDTAVYYCV |
| | | KGGQGAMDYWGQGTTVTVSS |
| 829 | PRT | SYYMS |
| 830 | PRT | TIIKSGGYAYYPDSVKD |
| 831 | PRT | GGQGAMDY |
| 832 | PRT | EIVLTQSPATLSLSPGERATLSCRASQSIGDYLHWYQQKPGQAPRLLIYY |
| | | ASQSISGIPARFSGSGSGTDFTLTISSLEPEDFAVYYCQQGYSFPYTFGGGT |
| | | KLEIK |
| 833 | PRT | RASQSIGDYLH |
| 834 | PRT | YASQSIS |
| 835 | PRT | QQGYSFPYT |
| 836 | DNA | GAAGTGCAGCTGGTGGAATCTGGCGGAGGACTGGTTCAGCTCGGCGG |
| | | ATCTCTGAGACTGTCTTGTGCCGCCAGCGGCTTCACCTTCAGCAGCTA |
| | | CTACATGAGCTGGGTCCGACAGGCCCCTGGCAAAGGACTTGAATGGG |
| | | TGTCCACCATCATCAAGAGCGGCGGCTACGCCTACTATCCCGACAGC |
| | | GTGAAGGACCGGTTCACCATCTCCAGAGACAACAGCAAGAACACCCT |
| | | GTACCTGCAGATGAACAGCCTGAGAGCCGAGGACACCGCCGTGTACT |
| | | ACTGTGTGAAAGGTGGACAGGGCGCCATGGACTATTGGGGCCAGGG |
| | | AACAACAGTGACCGTGTCCTCA |
| 837 | DNA | GAGATTGTGCTGACACAGTCTCCCGCCACACTGTCTCTTAGCCCTGGC |
| | | GAAAGAGCCACACTGAGCTGTAGAGCCAGCCAGAGCATCGGCGATT |
| | | ACCTGCACTGGTATCAGCAGAAGCCTGGACAGGCCCCTCGGCTGCTG |
| | | ATCTACTATGCCAGCCAGTCCATCAGCGGCATCCCCGCCAGATTTTCT |
| | | GGCAGCGGCTCTGGCACCGATTTCACCCTGACCATAAGCAGCCTGGA |
| | | ACCTGAGGACTTCGCCGTGTACTACTGCCAGCAGGGCTACAGCTTCC |
| | | CCTACACATTTGGCGGAGGCACCAAGCTGGAAATCAAA |
| 838 | PRT | EVQLVESGGGLVQLGGSLRLSCAASGFTFSSYYMSWVRQAPGKGLEWV |
| | | STIIKSGGYAYYPDSVKDRFTISRDNSKNTLYLQMNSLRAEDTAVYYCV |
| | | KGGQGAMDYWGQGTTVTVSSASTKGPSVFPLAPSSKSTSGGTAALGCL |
| | | VKDYFPEPVTVSWNSGALTSGVHTFPAVLQSSGLYSLSSVVTVPSSSLGT |
| | | QTYICNVNHKPSNTKVDKKVEPKSCDKTHTCPPCPAPELLGGPSVFLFPP |
| | | KPKDTLMISRTPEVTCVVVDVSHEDPEVKFNWYVDGVEVHNAKTKPRE |
| | | EQYNSTYRVVSVLTVLHQDWLNGKEYKCKVSNKALPAPIEKTISKAKG |
| | | QPREPQVYTLPPSRDELTKNQVSLTCLVKGFYPSDIAVEWESNGQPENN |
| | | YKTTPPVLDSDGSFFLYSKLTVDKSRWQQGNVFSCSVMHEALHNHYTQ |
| | | KSLSLSPGK |
| 839 | PRT | EIVLTQSPATLSLSPGERATLSCRASQSIGDYLHWYQQKPGQAPRLLIYY |
| | | ASQSISGIPARFSGSGSGTDFTLTISSLEPEDFAVYYCQQGYSFPYTFGGGT |
| | | KLEIKRTVAAPSVFIFPPSDEQLKSGTASVVCLLNNFYPREAKVQWKVD |
| | | NALQSGNSQESVTEQDSKDSTYSLSSTLTLSKADYEKHKVYACEVTHQG |
| | | LSSPVTKSFNRGEC |
| 840 | DNA | GAAGTGCAGCTGGTGGAATCTGGCGGAGGACTGGTTCAGCTCGGCGG |
| | | ATCTCTGAGACTGTCTTGTGCCGCCAGCGGCTTCACCTTCAGCAGCTA |
| | | CTACATGAGCTGGGTCCGACAGGCCCCTGGCAAAGGACTTGAATGGG |
| | | TGTCCACCATCATCAAGAGCGGCGGCTACGCCTACTATCCCGACAGC |
| | | GTGAAGGACCGGTTCACCATCTCCAGAGACAACAGCAAGAACACCCT |
| | | GTACCTGCAGATGAACAGCCTGAGAGCCGAGGACACCGCCGTGTACT |
| | | ACTGTGTGAAAGGTGGACAGGGCGCCATGGACTATTGGGGCCAGGG |
| | | AACAACAGTGACCGTGTCCTCAGCCAGCACCAAGGGCCCCAGCGTGT |
| | | TCCCTCTGGCCCCTAGCAGCAAGAGCACATCTGGCGGAACAGCCGCC |
| | | CTGGGCTGCCTCGTGAAGGACTACTTTCCCGAGCCCGTGACCGTGTC |
| | | CTGGAACTCTGGCGCTCTGACAAGCGGCGTGCACACCTTTCCAGCCG |
| | | TGCTGCAGAGCAGCGGCCTGTACTCTCTGAGCAGCGTCGTGACAGTG |
| | | CCCAGCAGCTCTCTGGGCACCCAGACCTACATCTGCAACGTGAACCA |
| | | CAAGCCCAGCAACACCAAGGTGGACAAGAAGGTGGAACCCAAGAGC |
| | | TGCGACAAGACCCACACCTGTCCCCCTTGTCCTGCCCCCGAACTGCTG |
| | | GGAGGCCCTTCCGTGTTCCTGTTCCCCCCAAAGCCCAAGGACACCCT |
| | | GATGATCAGCCGGACCCCCGAAGTGACCTGCGTGGTGGTGGATGTGT |
| | | CCCACGAGGACCCTGAAGTGAAGTTCAATTGGTACGTGGACGGCGTG |
| | | GAAGTGCACAACGCCAAGACCAAGCCTAGAGAGGAACAGTACAACA |
| | | GCACCTACCGGGTGGTGTCCGTGCTGACAGTGCTGCACCAGGACTGG |
| | | CTGAACGGCAAAGAGTACAAGTGCAAGGTGTCCAACAAGGCCCTGC |
| | | CTGCCCCCATCGAGAAAACCATCAGCAAGGCCAAGGGCCAGCCCCG |
| | | CGAACCCCAGGTGTACACACTGCCCCCAAGCAGGGACGAGCTGACC |
| | | AAGAACCAGGTGTCCCTGACCTGTCTCGTGAAAGGCTTCTACCCCTC |
| | | CGATATCGCCGTGGAATGGGAGAGCAACGGCCAGCCCGAGAACAAC |
| | | TACAAGACCACCCCCCCTGTGCTGGACAGCGACGGCTCATTCTTCCT |
| | | GTACAGCAAGCTGACCGTGGACAAGTCCCGGTGGCAGCAGGGCAAC |
| | | GTGTTCAGCTGCAGCGTGATGCACGAGGCCCTGCACAACCACTACAC |
| | | CCAGAAGTCCCTGAGCCTGAGCCCTGGCAAG |
| 841 | DNA | GAGATTGTGCTGACACAGTCTCCCGCCACACTGTCTCTTAGCCCTGGC |
| | | GAAAGAGCCACACTGAGCTGTAGAGCCAGCCAGAGCATCGGCGATT |
| | | ACCTGCACTGGTATCAGCAGAAGCCTGGACAGGCCCCTCGGCTGCTG |
| | | ATCTACTATGCCAGCCAGTCCATCAGCGGCATCCCCGCCAGATTTTCT |
| | | GGCAGCGGCTCTGGCACCGATTTCACCCTGACCATAAGCAGCCTGGA |
| | | ACCTGAGGACTTCGCCGTGTACTACTGCCAGCAGGGCTACAGCTTCC |
| | | CCTACACATTTGGCGGAGGCACCAAGCTGGAAATCAAACGAACCGTG |
| | | GCCGCTCCCAGCGTGTTCATCTTCCCACCTAGCGACGAGCAGCTGAA |
| | | GTCCGGCACAGCCTCTGTCGTGTGCCTGCTGAACAACTTCTACCCCCG |
| | | CGAGGCCAAGGTGCAGTGGAAGGTGGACAATGCCCTGCAGAGCGGC |
| | | AACAGCCAGGAAAGCGTGACCGAGCAGGACAGCAAGGACTCCACCT |
| | | ACAGCCTGAGCAGCACCCTGACCCTGAGCAAGGCCGACTACGAGAA |
| | | GCACAAGGTGTACGCCTGCGAAGTGACCCACCAGGGCCTGTCTAGCC |
| | | CCGTGACCAAGAGCTTCAACCGGGGCGAGTGT |
| 842 | PRT | EVQLVESGGGLLQLGGSLRLSCAASGFTFSSYYMSWVRQAPGKGLEWV |
| | | STIIKSGGYAYYPDSVKDRFTISRDNSKNTLNLQMNSLRAEDTAVYYCV |
| | | KGGQGAMDYWGQGTTVTVSS |
| 843 | PRT | SYYMS |
| 844 | PRT | TIIKSGGYAYYPDSVKD |
| 845 | PRT | GGQGAMDY |
| 846 | PRT | EIVLTQSPATLSLSPGERATLSCRASQSIGDYLHWYQQKPGQAPRLLIKY |
| | | ASQSISGIPARFSGSGSGTDFTLTISSLEPEDFAVYYCQQGYSFPYTFGGGT |
| | | KLEIK |
| 847 | PRT | RASQSIGDYLH |
| 848 | PRT | YASQSIS |
| 849 | PRT | QQGYSFPYT |
| 850 | DNA | GAAGTGCAGCTGGTGGAATCTGGCGGAGGACTGCTGCAGCTTGGCGG |
| | | ATCTCTGAGACTGTCTTGTGCCGCCAGCGGCTTCACCTTCAGCAGCTA |
| | | CTACATGAGCTGGGTCCGACAGGCCCCTGGCAAAGGACTTGAATGGG |
| | | TGTCCACCATCATCAAGAGCGGCGGCTACGCCTACTATCCCGACAGC |
| | | GTGAAGGACCGGTTCACCATCTCCAGAGACAACAGCAAGAACACCCT |
| | | GAACCTGCAGATGAACAGCCTGAGAGCCGAGGACACCGCCGTGTAC |
| | | TACTGTGTGAAAGGTGGACAGGGCGCCATGGACTATTGGGGCCAGG |
| | | GAACAACAGTGACCGTGTCCTCA |
| 851 | DNA | GAGATTGTGCTGACACAGTCTCCCGCCACACTGTCTCTTAGCCCTGGC |
| | | GAAAGAGCCACACTGAGCTGTAGAGCCAGCCAGAGCATCGGCGATT |
| | | ACCTGCACTGGTATCAGCAGAAGCCTGGACAGGCCCCTCGGCTGCTG |
| | | ATTAAGTACGCCAGCCAGTCCATCAGCGGCATCCCTGCCAGATTTTCT |
| | | GGCAGCGGCTCTGGCACCGATTTCACCCTGACCATAAGCAGCCTGGA |
| | | ACCTGAGGACTTCGCCGTGTACTACTGCCAGCAGGGCTACAGCTTCC |
| | | CCTACACATTTGGCGGAGGCACCAAGCTGGAAATCAAA |
| 852 | PRT | EVQLVESGGGLLQLGGSLRLSCAASGFTFSSYYMSWVRQAPGKGLEWV |
| | | STIIKSGGYAYYPDSVKDRFTISRDNSKNTLNLQMNSLRAEDTAVYYCV |
| | | KGGQGAMDYWGQGTTVTVSSASTKGPSVFPLAPSSKSTSGGTAALGCL |
| | | VKDYFPEPVTVSWNSGALTSGVHTFPAVLQSSGLYSLSSVVTVPSSSLGT |
| | | QTYICNVNHKPSNTKVDKKVEPKSCDKTHTCPPCPAPELLGGPSVFLFPP |
| | | KPKDTLMISRTPEVTCVVVDVSHEDPEVKFNWYVDGVEVHNAKTKPRE |
| | | EQYNSTYRVVSVLTVLHQDWLNGKEYKCKVSNKALPAPIEKTISKAKG |
| | | QPREPQVYTLPPSRDELTKNQVSLTCLVKGFYPSDIAVEWESNGQPENN |
| | | YKTTPPVLDSDGSFFLYSKLTVDKSRWQQGNVFSCSVMHEALHNHYTQ |
| | | KSLSLSPGK |
| 853 | PRT | EIVLTQSPATLSLSPGERATLSCRASQSIGDYLHWYQQKPGQAPRLLIKY |
| | | ASQSISGIPARFSGSGSGTDFTLTISSLEPEDFAVYYCQQGYSFPYTFGGGT |
| | | KLEIKRTVAAPSVFIFPPSDEQLKSGTASVVCLLNNFYPREAKVQWKVD |
| | | NALQSGNSQESVTEQDSKDSTYSLSSTLTLSKADYEKHKVYACEVTHQG |
| | | LSSPVTKSFNRGEC |
| 854 | DNA | GAAGTGCAGCTGGTGGAATCTGGCGGAGGACTGCTGCAGCTTGGCGG |
| | | ATCTCTGAGACTGTCTTGTGCCGCCAGCGGCTTCACCTTCAGCAGCTA |
| | | CTACATGAGCTGGGTCCGACAGGCCCCTGGCAAAGGACTTGAATGGG |
| | | TGTCCACCATCATCAAGAGCGGCGGCTACGCCTACTATCCCGACAGC |
| | | GTGAAGGACCGGTTCACCATCTCCAGAGACAACAGCAAGAACACCCT |
| | | GAACCTGCAGATGAACAGCCTGAGAGCCGAGGACACCGCCGTGTAC |
| | | TACTGTGTGAAAGGTGGACAGGGCGCCATGGACTATTGGGGCCAGG |
| | | GAACAACAGTGACCGTGTCCTCAGCCAGCACCAAGGGCCCCAGCGTG |
| | | TTCCCTCTGGCCCCTAGCAGCAAGAGCACATCTGGCGGAACAGCCGC |
| | | CCTGGGCTGCCTCGTGAAGGACTACTTTCCCGAGCCCGTGACCGTGT |
| | | CCTGGAACTCTGGCGCTCTGACAAGCGGCGTGCACACCTTTCCAGCC |
| | | GTGCTGCAGAGCAGCGGCCTGTACTCTCTGAGCAGCGTCGTGACAGT |
| | | GCCCAGCAGCTCTCTGGGCACCCAGACCTACATCTGCAACGTGAACC |
| | | ACAAGCCCAGCAACACCAAGGTGGACAAGAAGGTGGAACCCAAGAG |
| | | CTGCGACAAGACCCACACCTGTCCCCCTTGTCCTGCCCCCGAACTGCT |
| | | GGGAGGCCCTTCCGTGTTCCTGTTCCCCCCAAAGCCCAAGGACACCC |
| | | TGATGATCAGCCGGACCCCCGAAGTGACCTGCGTGGTGGTGGATGTG |
| | | TCCCACGAGGACCCTGAAGTGAAGTTCAATTGGTACGTGGACGGCGT |
| | | GGAAGTGCACAACGCCAAGACCAAGCCTAGAGAGGAACAGTACAAC |
| | | AGCACCTACCGGGTGGTGTCCGTGCTGACAGTGCTGCACCAGGACTG |
| | | GCTGAACGGCAAAGAGTACAAGTGCAAGGTGTCCAACAAGGCCCTG |
| | | CCTGCCCCCATCGAGAAAACCATCAGCAAGGCCAAGGGCCAGCCCC |
| | | GCGAACCCCAGGTGTACACACTGCCCCCAAGCAGGGACGAGCTGAC |
| | | CAAGAACCAGGTGTCCCTGACCTGTCTCGTGAAAGGCTTCTACCCCT |
| | | CCGATATCGCCGTGGAATGGGAGAGCAACGGCCAGCCCGAGAACAA |
| | | CTACAAGACCACCCCCCCTGTGCTGGACAGCGACGGCTCATTCTTCCT |
| | | GTACAGCAAGCTGACCGTGGACAAGTCCCGGTGGCAGCAGGGCAAC |
| | | GTGTTCAGCTGCAGCGTGATGCACGAGGCCCTGCACAACCACTACAC |
| | | CCAGAAGTCCCTGAGCCTGAGCCCTGGCAAG |
| 855 | DNA | GAGATTGTGCTGACACAGTCTCCCGCCACACTGTCTCTTAGCCCTGGC |
| | | GAAAGAGCCACACTGAGCTGTAGAGCCAGCCAGAGCATCGGCGATT |
| | | ACCTGCACTGGTATCAGCAGAAGCCTGGACAGGCCCCTCGGCTGCTG |
| | | ATTAAGTACGCCAGCCAGTCCATCAGCGGCATCCCTGCCAGATTTTCT |
| | | GGCAGCGGCTCTGGCACCGATTTCACCCTGACCATAAGCAGCCTGGA |
| | | ACCTGAGGACTTCGCCGTGTACTACTGCCAGCAGGGCTACAGCTTCC |
| | | CCTACACATTTGGCGGAGGCACCAAGCTGGAAATCAAACGAACCGTG |
| | | GCCGCTCCCAGCGTGTTCATCTTCCCACCTAGCGACGAGCAGCTGAA |
| | | GTCCGGCACAGCCTCTGTCGTGTGCCTGCTGAACAACTTCTACCCCCG |
| | | CGAGGCCAAGGTGCAGTGGAAGGTGGACAATGCCCTGCAGAGCGGC |
| | | AACAGCCAGGAAAGCGTGACCGAGCAGGACAGCAAGGACTCCACCT |
| | | ACAGCCTGAGCAGCACCCTGACCCTGAGCAAGGCCGACTACGAGAA |
| | | GCACAAGGTGTACGCCTGCGAAGTGACCCACCAGGGCCTGTCTAGCC |
| | | CCGTGACCAAGAGCTTCAACCGGGGCGAGTGT |
| 856 | PRT | EVQLLESGGGLVQPGGSLRLSCAASGFTFRSYAVHWVRQAPGKGLEWV |
| | | SSTEGSGVGTSYTDSVKGRFTISRDNSKNTLYLQMNSLRAEDTAVYYCA |
| | | RMLGGGNPLDYLDYWGQGTLVTVSS |
| 857 | PRT | SYAVH |
| 858 | PRT | STEGSGVGTSYTDSVKG |
| 859 | PRT | MLGGGNPLDYLDY |
| 860 | PRT | QSVLTQPPSASGTPGQRVTISCSGSSSNLGEGYDVHWYQQLPGKAPKLLI |
| | | YYSDFRPSGVSDRFSGSKSGTSASLAISGLQSEDEADYYCAAWDDSLSSQ |
| | | VFGGGTQVTVL |
| 861 | PRT | SGSSSNLGEGYDVH |
| 862 | PRT | YSDFRPS |
| 863 | PRT | AAWDDSLSSQV |
| 864 | DNA | GAAGTTCAGCTGCTGGAATCTGGCGGCGGACTGGTTCAACCTGGCGG |
| | | ATCTCTGAGACTGAGCTGTGCCGCCAGCGGCTTCACCTTTAGAAGCT |
| | | ATGCCGTGCACTGGGTCCGACAGGCCCCTGGAAAAGGACTGGAATG |
| | | GGTGTCCAGCACCGAAGGCTCTGGCGTGGGCACAAGCTACACCGATT |
| | | CTGTGAAGGGCAGATTCACCATCAGCCGGGACAACAGCAAGAACAC |
| | | CCTGTACCTGCAGATGAACAGCCTGAGAGCCGAGGACACCGCCGTGT |
| | | ACTACTGTGCCAGAATGCTCGGCGGAGGCAACCCTCTGGACTACCTG |
| | | GATTATTGGGGCCAGGGCACCCTGGTCACAGTCTCTTCA |
| 865 | DNA | CAGTCTGTTCTGACACAGCCTCCTAGCGCCTCTGGCACACCTGGACA |
| | | GAGAGTGACCATCAGCTGTAGCGGCAGCAGCTCCAATCTCGGCGAGG |
| | | GCTATGACGTGCACTGGTATCAGCAGCTGCCTGGCAAGGCCCCTAAA |
| | | CTGCTGATCTACTACAGCGACTTCAGACCCAGCGGCGTGTCCGATAG |
| | | ATTCAGCGGCTCTAAGAGCGGCACATCTGCCAGCCTGGCCATCTCTG |
| | | GACTGCAGAGCGAAGATGAGGCCGACTACTATTGCGCCGCCTGGGAT |
| | | GATAGCCTGAGCAGCCAAGTTTTTGGCGGCGGAACCCAAGTGACCGT |
| | | GCTA |
| 866 | PRT | EVQLLESGGGLVQPGGSLRLSCAASGFTFRSYAVHWVRQAPGKGLEWV |
| | | SSTEGSGVGTSYTDSVKGRFTISRDNSKNTLYLQMNSLRAEDTAVYYCA |
| | | RMLGGGNPLDYLDYWGQGTLVTVSSASTKGPSVFPLAPSSKSTSGGTAA |
| | | LGCLVKDYFPEPVTVSWNSGALTSGVHTFPAVLQSSGLYSLSSVVTVPSS |
| | | SLGTQTYICNVNHKPSNTKVDKKVEPKSCDKTHTCPPCPAPELLGGPSVF |
| | | LFPPKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEKTISK |
| | | AKGQPREPQVYTLPPSRDELTKNQVSLTCLVKGFYPSDIAVEWESNGQP |
| | | ENNYKTTPPVLDSDGSFFLYSKLTVDKSRWQQGNVFSCSVMHEALHNH |
| | | YTQKSLSLSPGK |
| 867 | PRT | QSVLTQPPSASGTPGQRVTISCSGSSSNLGEGYDVHWYQQLPGKAPKLLI |
| | | YYSDFRPSGVSDRFSGSKSGTSASLAISGLQSEDEADYYCAAWDDSLSSQ |
| | | VFGGGTQVTVLGQPKAAPSVTLFPPSSEELQANKATLVCLISDFYPGAVT |
| | | VAWKADSSPVKAGVETTTPSKQSNNKYAASSYLSLTPEQWKSHRSYSC |
| | | QVTHEGSTVEKTVAPTECS |
| 868 | DNA | GAAGTTCAGCTGCTGGAATCTGGCGGCGGACTGGTTCAACCTGGCGG |
| | | ATCTCTGAGACTGAGCTGTGCCGCCAGCGGCTTCACCTTTAGAAGCT |
| | | ATGCCGTGCACTGGGTCCGACAGGCCCCTGGAAAAGGACTGGAATG |
| | | GGTGTCCAGCACCGAAGGCTCTGGCGTGGGCACAAGCTACACCGATT |
| | | CTGTGAAGGGCAGATTCACCATCAGCCGGGACAACAGCAAGAACAC |
| | | CCTGTACCTGCAGATGAACAGCCTGAGAGCCGAGGACACCGCCGTGT |
| | | ACTACTGTGCCAGAATGCTCGGCGGAGGCAACCCTCTGGACTACCTG |
| | | GATTATTGGGGCCAGGGCACCCTGGTCACAGTCTCTTCAGCCAGCAC |
| | | CAAGGGCCCCAGCGTGTTCCCTCTGGCCCCTAGCAGCAAGAGCACAT |
| | | CTGGCGGAACAGCCGCCCTGGGCTGCCTCGTGAAGGACTACTTTCCC |
| | | GAGCCCGTGACCGTGTCCTGGAACTCTGGCGCTCTGACAAGCGGCGT |
| | | GCACACCTTTCCAGCCGTGCTGCAGAGCAGCGGCCTGTACTCTCTGA |
| | | GCAGCGTCGTGACAGTGCCCAGCAGCTCTCTGGGCACCCAGACCTAC |
| | | ATCTGCAACGTGAACCACAAGCCCAGCAACACCAAGGTGGACAAGA |
| | | AGGTGGAACCCAAGAGCTGCGACAAGACCCACACCTGTCCCCCTTGT |
| | | CCTGCCCCCGAACTGCTGGGAGGCCCTTCCGTGTTCCTGTTCCCCCCA |
| | | AAGCCCAAGGACACCCTGATGATCAGCCGGACCCCCGAAGTGACCTG |
| | | CGTGGTGGTGGATGTGTCCCACGAGGACCCTGAAGTGAAGTTCAATT |
| | | GGTACGTGGACGGCGTGGAAGTGCACAACGCCAAGACCAAGCCTAG |
| | | AGAGGAACAGTACAACAGCACCTACCGGGTGGTGTCCGTGCTGACA |
| | | GTGCTGCACCAGGACTGGCTGAACGGCAAAGAGTACAAGTGCAAGG |
| | | TGTCCAACAAGGCCCTGCCTGCCCCCATCGAGAAAACCATCAGCAAG |
| | | GCCAAGGGCCAGCCCCGCGAACCCCAGGTGTACACACTGCCCCCAAG |
| | | CAGGGACGAGCTGACCAAGAACCAGGTGTCCCTGACCTGTCTCGTGA |
| | | AAGGCTTCTACCCCTCCGATATCGCCGTGGAATGGGAGAGCAACGGC |
| | | CAGCCCGAGAACAACTACAAGACCACCCCCCCTGTGCTGGACAGCGA |
| | | CGGCTCATTCTTCCTGTACAGCAAGCTGACCGTGGACAAGTCCCGGT |
| | | GGCAGCAGGGCAACGTGTTCAGCTGCAGCGTGATGCACGAGGCCCTG |
| | | CACAACCACTACACCCAGAAGTCCCTGAGCCTGAGCCCTGGCAAG |
| 869 | DNA | CAGTCTGTTCTGACACAGCCTCCTAGCGCCTCTGGCACACCTGGACA |
| | | GAGAGTGACCATCAGCTGTAGCGGCAGCAGCTCCAATCTCGGCGAGG |
| | | GCTATGACGTGCACTGGTATCAGCAGCTGCCTGGCAAGGCCCCTAAA |
| | | CTGCTGATCTACTACAGCGACTTCAGACCCAGCGGCGTGTCCGATAG |
| | | ATTCAGCGGCTCTAAGAGCGGCACATCTGCCAGCCTGGCCATCTCTG |
| | | GACTGCAGAGCGAAGATGAGGCCGACTACTATTGCGCCGCCTGGGAT |
| | | GATAGCCTGAGCAGCCAAGTTTTTGGCGGCGGAACCCAAGTGACCGT |
| | | GCTAGGCCAGCCTAAAGCCGCCCCTAGCGTGACCCTGTTCCCTCCAA |
| | | GCAGCGAGGAACTGCAGGCCAACAAGGCCACCCTCGTGTGCCTGATC |
| | | AGCGACTTCTATCCTGGCGCCGTGACCGTGGCCTGGAAGGCCGATAG |
| | | CTCTCCTGTGAAGGCCGGCGTGGAAACCACCACCCCTAGCAAGCAGA |
| | | GCAACAACAAATACGCCGCCAGCAGCTACCTGAGCCTGACCCCCGAG |
| | | CAGTGGAAGTCCCACAGATCCTACAGCTGCCAAGTGACCCACGAGGG |
| | | CAGCACCGTGGAAAAGACAGTGGCCCCTACCGAGTGCAGC |
| 870 | PRT | EVQLLESGGGLVQPGGSLRLSCAASGFTFRSYAVHWVRQAPGKGLEWV |
| | | SSTEGSGVGTSYTDSVKGRFTISRDNSKNTLYLQMNSLRAEDTAVYYCA |
| | | RMLGGGNPLDYLDYWGQGTLVTVSS |
| 871 | PRT | SYAVH |
| 872 | PRT | STEGSGVGTSYTDSVKG |
| 873 | PRT | MLGGGNPLDYLDY |
| 874 | PRT | QSVLTQPPSASGTPGQRVTISCSGSSSNLGEGYDVHWYQQLPGKAPKLLI |
| | | YYSDFRPSGVSDRFSGSKSGTSASLAISGLQSEDEADYYCAAWDDSLSSQ |
| | | VFGGGTQVTVL |
| 875 | PRT | SGSSSNLGEGYDVH |
| 876 | PRT | YSDFRPS |
| 877 | PRT | AAWDDSLSSQV |
| 878 | DNA | GAAGTTCAGCTGCTGGAATCTGGCGGCGGACTGGTTCAACCTGGCGG |
| | | ATCTCTGAGACTGAGCTGTGCCGCCAGCGGCTTCACCTTTAGAAGCT |
| | | ATGCCGTGCACTGGGTCCGACAGGCCCCTGGAAAAGGACTGGAATG |
| | | GGTGTCCAGCACCGAAGGCTCTGGCGTGGGCACAAGCTACACCGATT |
| | | CTGTGAAGGGCAGATTCACCATCAGCCGGGACAACAGCAAGAACAC |
| | | CCTGTACCTGCAGATGAACAGCCTGAGAGCCGAGGACACCGCCGTGT |
| | | ACTACTGTGCCAGAATGCTCGGCGGAGGCAACCCTCTGGACTACCTG |
| | | GATTATTGGGGCCAGGGCACCCTGGTCACAGTCTCTTCA |
| 879 | DNA | CAGTCTGTTCTGACACAGCCTCCTAGCGCCTCTGGCACACCTGGACA |
| | | GAGAGTGACCATCAGCTGTAGCGGCAGCAGCTCCAATCTCGGCGAGG |
| | | GCTATGACGTGCACTGGTATCAGCAGCTGCCTGGCAAGGCCCCTAAA |
| | | CTGCTGATCTACTACAGCGACTTCAGACCCAGCGGCGTGTCCGATAG |
| | | ATTCAGCGGCTCTAAGAGCGGCACATCTGCCAGCCTGGCCATCTCTG |
| | | GACTGCAGAGCGAAGATGAGGCCGACTACTATTGCGCCGCCTGGGAT |
| | | GATAGCCTGAGCAGCCAAGTTTTTGGCGGCGGAACCCAAGTGACCGT |
| | | GCTA |
| 880 | PRT | EVQLLESGGGLVQPGGSLRLSCAASGFTFRSYAVHWVRQAPGKGLEWV |
| | | SSTEGSGVGTSYTDSVKGRFTISRDNSKNTLYLQMNSLRAEDTAVYYCA |
| | | RMLGGGNPLDYLDYWGQGTLVTVSSASTKGPSVFPLAPSSKSTSGGTAA |
| | | LGCLVKDYFPEPVTVSWNSGALTSGVHTFPAVLQSSGLYSLSSVVTVPSS |
| | | SLGTQTYICNVNHKPSNTKVDKKVEPKSCAAGSEQKLISEEDLSGSAAA |
| | | HHHHHH |
| 881 | PRT | QSVLTQPPSASGTPGQRVTISCSGSSSNLGEGYDVHWYQQLPGKAPKLLI |
| | | YYSDFRPSGVSDRFSGSKSGTSASLAISGLQSEDEADYYCAAWDDSLSSQ |
| | | VFGGGTQVTVLGQPKAAPSVTLFPPSSEELQANKATLVCLISDFYPGAVT |
| | | VAWKADSSPVKAGVETTTPSKQSNNKYAASSYLSLTPEQWKSHRSYSC |
| | | QVTHEGSTVEKTVAPTECS |
| 882 | DNA | GAAGTTCAGCTGCTGGAATCTGGCGGCGGACTGGTTCAACCTGGCGG |
| | | ATCTCTGAGACTGAGCTGTGCCGCCAGCGGCTTCACCTTTAGAAGCT |
| | | ATGCCGTGCACTGGGTCCGACAGGCCCCTGGAAAAGGACTGGAATG |
| | | GGTGTCCAGCACCGAAGGCTCTGGCGTGGGCACAAGCTACACCGATT |
| | | CTGTGAAGGGCAGATTCACCATCAGCCGGGACAACAGCAAGAACAC |
| | | CCTGTACCTGCAGATGAACAGCCTGAGAGCCGAGGACACCGCCGTGT |
| | | ACTACTGTGCCAGAATGCTCGGCGGAGGCAACCCTCTGGACTACCTG |
| | | GATTATTGGGGCCAGGGCACCCTGGTCACAGTCTCTTCAGCCTCCAC |
| | | CAAGGGCCCATCGGTGTTCCCCCTGGCACCCTCCTCCAAGAGCACCT |
| | | CTGGGGGCACAGCGGCCCTGGGCTGCCTGGTCAAGGACTACTTCCCC |
| | | GAACCGGTGACGGTGTCGTGGAACTCAGGCGCCCTGACCAGCGGCGT |
| | | GCACACCTTCCCGGCTGTCCTACAGTCCTCAGGACTCTACTCCCTCAG |
| | | CAGCGTGGTGACCGTGCCCTCCAGCAGCTTGGGCACCCAGACCTACA |
| | | TCTGCAACGTGAATCACAAGCCCAGCAACACCAAGGTGGACAAGAA |
| | | AGTTGAGCCCAAATCTTGTGCAGCGGGTTCTGAACAAAAACTCATCT |
| | | CAGAAGAGGATCTGTCTGGATCAGCGGCCGCCCATCATCATCATCAT |
| | | CAT |
| 883 | DNA | CAGTCTGTTCTGACACAGCCTCCTAGCGCCTCTGGCACACCTGGACA |
| | | GAGAGTGACCATCAGCTGTAGCGGCAGCAGCTCCAATCTCGGCGAGG |
| | | GCTATGACGTGCACTGGTATCAGCAGCTGCCTGGCAAGGCCCCTAAA |
| | | CTGCTGATCTACTACAGCGACTTCAGACCCAGCGGCGTGTCCGATAG |
| | | ATTCAGCGGCTCTAAGAGCGGCACATCTGCCAGCCTGGCCATCTCTG |
| | | GACTGCAGAGCGAAGATGAGGCCGACTACTATTGCGCCGCCTGGGAT |
| | | GATAGCCTGAGCAGCCAAGTTTTTGGCGGCGGAACCCAAGTGACCGT |
| | | GCTAGGCCAGCCTAAAGCCGCCCCTAGCGTGACCCTGTTCCCTCCAA |
| | | GCAGCGAGGAACTGCAGGCCAACAAGGCCACCCTCGTGTGCCTGATC |
| | | AGCGACTTCTATCCTGGCGCCGTGACCGTGGCCTGGAAGGCCGATAG |
| | | CTCTCCTGTGAAGGCCGGCGTGGAAACCACCACCCCTAGCAAGCAGA |
| | | GCAACAACAAATACGCCGCCAGCAGCTACCTGAGCCTGACCCCCGAG |
| | | CAGTGGAAGTCCCACAGATCCTACAGCTGCCAAGTGACCCACGAGGG |
| | | CAGCACCGTGGAAAAGACAGTGGCCCCTACCGAGTGCAGC |
| 884 | PRT | NYQNGKNNVPRLKLSYKEMLESNNVITFNGLANSSSYHTFLLDEERSRL |
| | | YVGAKDHIFSFNLVNIKDFQKIAWPVSYTRRDECKWAGKDILRECANFI |
| | | KVLKVYNQTHLYACGTGAFHPICTYVGIGHHPEDNIFKLEDSHFENGRG |
| | | KSPYDPKLLTASLLIDGELYSGTAADFMGRDFAIFRTLGQHHPIRTEQHD |
| | | SRWLNDPRFISAHLIPESDNPEDDKVYFFFRENAIDGEHSGKATHARIGQI |
| | | CKNDFGGHRSLVNKWTTFLKARLICSVPGPNGIDTHFDELQDVFLMNSK |
| | | DPKNPIVYGVFTTSSNIFRGSAVCMYSMSDVRRVFLGPYAHRDGPNYQ |
| | | WVPFQGRVPYPRPGTCPSKTFGGFESTKDLPDDVITFARSHPAMYNPVFP |
| | | INNRPIMVKTDVNYQFTQIVVDRVDAEDGQYDVMFIGTDVGTVLKVVSI |
| | | PKETWHDLEEVLLEEMTVFREPTTISAMELSTKQQQLYVGSAAGVAQLP |
| | | LHRCDIYGKACAECCLARDPYCAWDGSSCSRYFPTAKRRTRRQDIRNG |
| | | DPLTHCSDGGIEGRMDHHHHHH |
| 885 | DNA | AACTATCAGAACGGCAAGAACAACGTGCCCCGGCTGAAGCTGAGCT |
| | | ACAAAGAGATGCTGGAAAGCAACAACGTGATCACCTTCAACGGCCT |
| | | GGCCAACAGCAGCAGCTACCACACCTTTCTGCTGGACGAGGAACGGT |
| | | CCAGACTGTACGTGGGAGCCAAGGACCACATCTTCAGCTTCAACCTG |
| | | GTCAACATCAAGGACTTCCAGAAAATCGCCTGGCCTGTGTCCTACAC |
| | | CAGACGGGATGAGTGTAAATGGGCCGGCAAGGACATCCTGAGAGAG |
| | | TGCGCCAACTTCATCAAGGTGCTGAAGGTGTACAATCAGACCCACCT |
| | | GTACGCCTGTGGCACCGGCGCTTTTCACCCTATCTGTACCTATGTCGG |
| | | CATCGGCCACCATCCTGAGGACAATATCTTCAAGCTCGAGGACAGCC |
| | | ACTTCGAGAACGGCAGAGGCAAGAGCCCCTACGATCCCAAACTGCTG |
| | | ACAGCCTCTCTGCTGATCGACGGCGAGCTGTATTCTGGCACAGCCGC |
| | | CGATTTCATGGGCAGAGACTTCGCCATCTTCAGAACCCTGGGCCAGC |
| | | ATCACCCCATCAGAACCGAGCAGCACGACAGCAGATGGCTGAACGA |
| | | CCCCAGATTCATCAGCGCCCATCTGATCCCCGAGAGCGACAACCCCG |
| | | AGGACGACAAGGTGTACTTCTTCTTCCGGGAAAACGCCATCGACGGG |
| | | GAGCACTCTGGAAAAGCCACACACGCCAGAATCGGCCAGATCTGCA |
| | | AGAACGACTTCGGCGGCCACAGATCCCTCGTGAACAAGTGGACCACC |
| | | TTCCTGAAGGCCCGGCTGATCTGTTCTGTGCCCGGACCTAATGGCATC |
| | | GATACCCACTTCGACGAGCTGCAGGACGTGTTCCTGATGAACAGCAA |
| | | GGACCCCAAGAATCCCATCGTGTACGGCGTGTTCACCACCAGCAGCA |
| | | ACATCTTTAGAGGCAGCGCCGTGTGCATGTACAGCATGTCCGATGTG |
| | | CGGAGAGTGTTTCTGGGCCCCTACGCTCACAGAGATGGCCCCAATTA |
| | | TCAGTGGGTGCCATTCCAGGGCAGAGTGCCCTATCCTAGACCTGGCA |
| | | CCTGTCCTAGCAAGACCTTTGGCGGCTTCGAGAGCACCAAGGACCTG |
| | | CCTGACGATGTGATTACCTTCGCCAGATCTCACCCCGCCATGTACAAC |
| | | CCTGTGTTCCCCATCAACAACAGGCCCATCATGGTCAAGACCGACGT |
| | | GAACTACCAGTTCACCCAGATCGTGGTGGACAGAGTGGATGCCGAGG |
| | | ACGGCCAGTACGACGTGATGTTCATCGGCACCGATGTGGGCACCGTG |
| | | CTGAAAGTGGTGTCTATCCCCAAAGAGACATGGCACGACCTGGAAGA |
| | | GGTGCTGCTGGAAGAGATGACCGTGTTCAGAGAGCCCACCACCATCT |
| | | CCGCCATGGAACTGAGCACAAAACAGCAACAGCTGTATGTGGGCTCC |
| | | GCCGCTGGTGTTGCTCAACTGCCTCTGCACAGATGCGACATCTACGG |
| | | CAAAGCCTGCGCCGAGTGTTGCCTGGCCAGAGATCCTTACTGTGCCT |
| | | GGGATGGCAGCAGCTGCAGCAGATACTTTCCCACCGCCAAGCGGAG |
| | | AACCAGACGGCAGGATATCAGAAACGGCGACCCTCTGACACACTGC |
| | | AGCGACGGTGGCATCGAGGGCCGCATGGATCATCATCATCACCATCA |
| | | T |

What is claimed is:

1. An isolated antibody or antigen-binding fragment thereof for binding to human Sema3A, wherein said isolated antibody or antigen-binding fragment thereof comprises:
a heavy chain antigen-binding region comprising:
an H-CDR1 comprising SEQ ID NO: 142,
an H-CDR2 comprising SEQ ID NO: 143, and
an H-CDR3 comprising SEQ ID NO: 144 and
a light chain antigen-binding region comprising:
an L-CDR1 comprising SEQ ID NO: 146,
an L-CDR2 comprising SEQ ID NO: 147, and
an L-CDR3 comprising SEQ ID NO: 148.

2. The isolated antibody or antigen-binding fragment according to claim 1 comprising a variable heavy chain domain that is at least 98% identical to SEQ ID NO: 141 and a variable light chain domain that is at least 98%, identical to SEQ ID NO: 145.

3. The isolated antibody or antigen-binding fragment according to claim 1 comprising a variable heavy chain domain comprising SEQ ID NO: 141 and a variable light chain domain comprising SEQ ID NO: 145.

4. The isolated antibody according to claim 1, wherein said isolated antibody is an IgG1 or an IgG4 antibody.

5. The isolated antibody according to claim 1 comprising a heavy chain comprising SEQ ID NO: 157 and a light chain comprising SEQ ID NO: 158.

6. The antigen-binding fragment according to claim 1, which is an scFv, Fab, Fab′ fragment or a F(ab′)2 fragment.

7. An antibody conjugate, comprising the isolated antibody or antigen binding fragment according to claim 1.

8. A pharmaceutical composition comprising the isolated antibody or antigen-binding fragment according to claim 1 or the antibody conjugate comprising the isolated antibody or antigen binding fragment.

9. A kit comprising the isolated antibody or antigen-binding fragment according to claim 1, a conjugate comprising the isolated antibody or antigen binding fragment, or the pharmaceutical composition comprising the isolated antibody or antigen binding fragment, or the conjugate comprising the isolated antibody or antigen binding fragment and instructions for use.
